(19) United States
(12) Patent Application Publication
(10) Pub. No.: US 2024/0174696 A1
(43) Pub. Date: May 30, 2024

(54) 2,8-DIAZASPIRO[4.5]DECANE COMPOUNDS

(71) Applicant: Genentech, Inc., South San Francisco, CA (US)

(72) Inventors: Steven John MCKERRALL, San Francisco, CA (US); Alice Rose WONG, Burlingame, CA (US); James John CRAWFORD, San Carlos, CA (US); Wendy LEE, San Ramon, CA (US); Kwong Wah LAI, Shanghai (CN); Guillaume PELLETIER, Montreal (CA); Stéphanie ROY, Montreal (CA); Dana Krystin WINTER, Montreal (CA); Mikiko OKUMURA, Burlingame, CA (US); Ruth Dorel, Millbrae, CA (US)

(73) Assignee: Genentech, Inc., South San Francisco, CA (US)

(21) Appl. No.: 18/526,572

(22) Filed: Dec. 1, 2023

Related U.S. Application Data
(63) Continuation of application No. PCT/CN2022/097025, filed on Jun. 3, 2022.

(30) Foreign Application Priority Data
Jun. 4, 2021 (WO) PCT/CN2021/098358

(51) Int. Cl. C07D 519/00; A61P 11/00

(52) U.S. Cl. CPC C07D 519/00; A61P 11/00

(57) ABSTRACT

Disclosed are 2,8-diazaspiro[4.5]decane compounds, including (pyrido[3,4-d]pyrimidin-4-yl)-2,8-diazaspiro[4.5]decane compounds, (2,6-naphthyridin-1-yl)-2,8-diazaspiro[4.5]decane compounds, and (1,7-naphthyridin-4-yl)-2,8-diazaspiro[4.5]decane compounds, that are inhibitors of LATS1/2, compositions containing these compounds, and methods for inhibiting LATS1/2 activity.

### CROSS-REFERENCE TO RELATED APPLICATIONS

This application claims the benefit of and priority to International Patent Application No. PCT/CN2021/098358 filed 4 Jun. 2021, the content of which application is incorporated herein by reference in its entirety.

### BACKGROUND OF THE INVENTION

Large tumor suppressor kinase 1 (LATS1) and large tumor suppressor kinase 2 (LATS2) are regulatory serine/threonine kinases in the Hippo pathway that constitutively phosphorylate the effector transcription factors Yes-associated protein (YAP) and transcriptional co-activator with PDZ-binding motif (TAZ), thereby inactivating them. When the Hippo pathway is active, a series of upstream factors phosphorylate the Hippo kinases MST1/2, which in turn phosphorylate LATS1/2. LATS1/2 phosphorylates YAP and TAZ, causing YAP and TAZ to be sequestered in the cytoplasm and degraded. When the Hippo pathway is inactive and LATS1/2 is eliminated, reduced, and/or not phosphorylated, YAP and TAZ are not phosphorylated and instead translocate to the nucleus. In the nucleus, YAP and TAZ complex with transcription factors, such the TEAD family of transcription factors, to regulate a series of downstream genes relevant to functions including cancer resistance, cell proliferation, apoptosis, and other cellular properties. Literature reports have also shown that YAP/TAZ activation after injury promotes tissue regeneration and repair in multiple cell types, including in lung-injury models. See e.g., LaCanna, R. et al. J Clin Invest. 2019; 129(5):2107-2122; and JCI Insight. 2019; 4 (14):e128674.

As a result, LATS1 and LATS2 pathway inactivation could represent an option for pharmacologic intervention in human diseases or conditions such as idiopathic pulmonary fibrosis (IPF) and acute respiratory distress syndrome (ARDS).

### BRIEF SUMMARY OF THE INVENTION

Disclosed are 2,8-diazaspiro[4.5]decane compounds, including (pyrido[3,4-d]pyrimidin-4-yl)-2,8-diazaspiro[4.5]decane compounds, (2,6-naphthyridin-1-yl)-2,8-diazaspiro[4.5]decane compounds, and (1,7-naphthyridin-4-yl)-2,8-diazaspiro[4.5]decane compounds, that are inhibitors of LATS1/2, compositions containing these compounds, and methods for inhibiting LATS1/2 in cells or a subject, promoting tissue regeneration after injury, and treating a disease, disorder or condition that can benefit from LATS1/2 inhibition.

In one aspect, provided is a compound of Formula (I), or any variation thereof such as Formula (IA), (IB) or (IC), or an N-oxide thereof, or a salt thereof (e.g., a pharmaceutically acceptable salt thereof), as detailed herein. Also provided is a pharmaceutical composition comprising a compound of Formula (I), or any variation thereof detailed herein, or a pharmaceutically acceptable salt thereof, and a pharmaceutically acceptable carrier or excipient.

In another aspect, provided is a method for promoting tissue regeneration after injury or treating a disease or condition that can benefit from LATS1/2 inhibition (e.g., ARDS), comprising administering to a subject in need thereof an effective amount of the compound of Formula (I), or any variation thereof such as Formula (IA), (IB) or (IC) detailed herein, or a pharmaceutically acceptable salt thereof. In some embodiments, the subject is a human.

Also provided is a compound of Formula (I), or any variation thereof such as Formula (IA), (IB) or (IC) detailed herein, or a pharmaceutically acceptable salt thereof, for use in a method of promoting tissue regeneration after injury or treating disease or condition that can benefit from LATS1/2 inhibition (e.g., ARDS).

Also provided is use of a compound of Formula (I), or any variation thereof such as Formula (IA), (IB) or (IC) detailed herein, or a pharmaceutically acceptable salt thereof, in a method detailed herein (e.g., promoting tissue regeneration after injury, or treatment of ARDS).

Also provided is use of a compound of Formula (I), or any variation thereof such as Formula (IA), (IB) or (IC) detailed herein, or a pharmaceutically acceptable salt thereof, for the manufacture of a medicament for use in a method detailed herein (e.g., promoting tissue regeneration after injury or treatment of ARDS).

Also provided is a kit for promoting tissue regeneration after injury or treating a disease or condition that can benefit from LATS1/2 inhibition (e.g., ARDS), the kit comprising a pharmaceutical composition comprising a the compound of Formula (I), or any variation thereof such as Formula (IA), (IB) or (IC) detailed herein, or a pharmaceutically acceptable salt thereof, and instructions for use.

In another aspect, provided is a method of making a compound of Formula (I) or any variation thereof such as Formula (IA), (IB) or (IC). Also provided are compound intermediates useful in synthesis of a compound of Formula (I), or any variation thereof such as Formula (IA), (IB) or (IC).

### DETAILED DESCRIPTION OF THE INVENTION

Disclosed herein, are compounds of Formula (I), or variations thereof such as Formulae (IA), (IB), (IC), (II-A), (II-B), (II-C), (III)-(IX), e.g., Compound Nos. 101-201 in Table 1, and pharmaceutical compositions thereof that are inhibitors of LATS1/2. As such, the compounds and compositions are useful in treating diseases, disorders or conditions that can benefit from LATS1/2 inhibition.

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

### Definitions

“Alkyl” as used herein refers to a saturated linear (i.e. unbranched) or branched univalent hydrocarbon chain or combination thereof, having the number of carbon atoms designated (i.e., C₁₋₁₀ means one to ten carbon atoms). Particular alkyl groups are those having 1 to 20 carbon atoms (a “C₁₋₂₀ alkyl”), having a 1 to 8 carbon atoms (a “C₁₋₈ alkyl”), having 1 to 6 carbon atoms (a “C₁₋₆ alkyl”), having 2 to 6 carbon atoms (a “C₂₋₆ alkyl”), or having 1 to 4 carbon atoms (a “C₁₋₄ alkyl”). Examples of alkyl group include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

“Alkenyl” as used herein refers to an unsaturated linear (i.e., unbranched) or branched univalent hydrocarbon chain or combination thereof, having at least one site of olefinic unsaturation (i.e., having at least one moiety of the formula C═C) and having the number of carbon atoms designated (i.e., C₂₋₁₀ means two to ten carbon atoms). The alkenyl group may be in “cis” or “trans” configurations, or alternatively in “E” or “Z” configurations. Particular alkenyl groups are those having 2 to 20 carbon atoms (a “C₂₋₂₀ alkenyl”), having a 2 to 8 carbon atoms (a “C₂₋₈ alkenyl”), having 2 to 6 carbon atoms (a “C₂₋₆ alkenyl”), or having 2 to 4 carbon atoms (a “C₂₋₄ alkenyl”). Example of alkenyl group include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, homologs and isomers thereof, and the like.

“Alkynyl” as used herein refers to an unsaturated linear (i.e. unbranched) or branched univalent hydrocarbon chain or combination thereof, having at least one site of acetylenic unsaturation (i.e., having at least one moiety of the formula C≡C) having the number of carbon atoms designated (i.e., C₂₋₁₀ means two to ten carbon atoms). Particular alkynyl groups are those having 2 to 20 carbon atoms (a “C₂₋₂₀ alkynyl”), having a 2 to 8 carbon atoms (a “C₂₋₈ alkynyl”), having 2 to 6 carbon atoms (a “C₂₋₆ alkynyl”), having 2 to 4 carbon atoms (a “C₂₋₄ alkynyl”). Examples of alkynyl groups include, but are not limited to, groups such as ethynyl (or acetylenyl), prop-1-ynyl, prop-2-ynyl (or propargyl), but-1-ynyl, but-2-ynyl, but-3-ynyl, homologs and isomers thereof, and the like.

“Alkylene” as used herein refers to the same residues as alkyl, but having bivalency. Particular alkylene groups are those having 1 to 6 carbon atoms (a “C₁₋₆ alkylene”), 1 to 5 carbon atoms (a “C₁₋₅ alkylene”), having 1 to 4 carbon atoms (a “C₁₋₄ alkylene”), or 1 to 3 carbon atoms (a “C₁₋₃ alkylene”). Examples of alkylene include, but are not limited to, groups such as methylene (—CH₂—), ethylene (—CH₂—CH₂—), 1,3-propylene (—CH₂—CH₂—CH₂—), 1,2-propylene (—CH(CH₃)—CH₂—), 1,4-butylene (—CH₂—CH₂—CH₂—CH₂—), and the like.

“Alkylidene” as used herein refers to the same residues as alkyl, but having bivalency at the attachment point and is attached to the parent structure via a double bond. Particular alkylidene groups are those having 1 to 6 carbon atoms (a “C₁₋₆ alkylidene”), 1 to 5 carbon atoms (a “C₁₋₅ alkylidene”), having 1 to 4 carbon atoms (a “C₁₋₄ alkylidene”), or 1 to 3 carbon atoms (a “C₁₋₃ alkylidene”). Examples of alkylidene include, but are not limited to, groups such as methylidene (═CH₂), ethylidene (═CH—CH₃), 1-propylidene (═CH—CH₂—CH₃), 2-propylidene (═C(CH₃)₂), 1-butylidene (═CH₂—CH₂—CH₂—CH₃), and the like.

“Cycloalkyl” as used herein refers to non-aromatic, saturated or unsaturated cyclic univalent hydrocarbon structures having the number of carbon atoms designated (i.e., (C₃-10 means three to ten carbon atoms). Cycloalkyl can consist of one ring, such as cyclohexyl, or multiple rings, such as adamantyl, but excludes aryl groups. A cycloalkyl comprising more than one ring may be fused, spiro, or bridged, or combinations thereof. Particular cycloalkyl groups are those having from 3 to 12 annular carbon atoms. A preferred cycloalkyl is a cyclic hydrocarbon having from 3 to 8 annular carbon atoms (a “C₃₋₈ cycloalkyl”), or having 3 to 6 carbon atoms (a “C₃₋₆ alkynyl”). Examples of cycloalkyl include, but are not limited to, cyclopropyl, cyclobutyl, cyclopentyl, cyclohyxyl, 1-cyclohexenyl, 3-cyclohexenyl, cycloheptyl, norbomyl, and the like.

“Aryl” as used herein refers to an unsaturated aromatic carbocyclic group having a single ring (e.g., phenyl) or multiple condensed rings (e.g., naphthyl or anthryl) which condensed rings may or may not be aromatic. Particular aryl groups are those having from 6 to 14 annular (i.e., ring) carbon atoms (a “C₆₋₁₄ aryl”). An aryl group having more than one ring where at least one ring is non-aromatic may be connected to the parent structure at either an aromatic ring position or at a non-aromatic ring position. In one variation, an aryl group having more than one ring where at least one ring is non-aromatic is connected to the parent structure at an aromatic ring position.

“Heteroaryl” as used herein refers to an unsaturated aromatic cyclic group having from 1 to 14 annular (i.e., ring) carbon atoms and at least one annular heteroatom, including but not limited to heteroatoms such as nitrogen, phosphorus, oxygen and sulfur. A heteroaryl group may have a single ring (e.g., pyridyl, furyl) or multiple condensed rings (e.g., indolizinyl, benzothienyl) which condensed rings may or may not be aromatic. Particular heteroaryl groups are 5- to 14-membered rings having 1 to 12 annular (i.e., ring) carbon atoms and 1 to 6 annular (i.e., ring) heteroatoms independently selected from nitrogen, phosphorus, oxygen and sulfur; 5 to 10-membered rings having 1 to 8 annular carbon atoms and 1 to 4 annular heteroatoms independently selected from nitrogen, phosphorus, oxygen and sulfur; and 5-, 6- or 7-membered rings having 1 to 5 annular carbon atoms and 1 to 4 annular heteroatoms independently selected from nitrogen, oxygen and sulfur. In one variation, heteroaryl include monocyclic aromatic 5-, 6- or 7-membered rings having from 1 to 6 annular carbon atoms and 1 to 4 annular heteroatoms independently selected from nitrogen, oxygen and sulfur. In another variation, heteroaryl includes polycyclic aromatic rings having from 1 to 12 annular carbon atoms and 1 to 6 annular heteroatoms independently selected from nitrogen, phosphorus, oxygen and sulfur. A heteroaryl group having more than one ring where at least one ring is non-aromatic may be connected to the parent structure at either an aromatic ring position or at a non-aromatic ring position. In one variation, a heteroaryl group having more than one ring where at least one ring is non-aromatic is connected to the parent structure at an aromatic ring position.

“Heterocycle”, “heterocyclic”, or “heterocyclyl” as used herein refers to a saturated or an unsaturated non-aromatic cyclic group having a single ring or multiple condensed rings, and having from 1 to 14 annular (i.e., ring) carbon atoms and from 1 to 6 annular (i.e., ring) heteroatoms, such as nitrogen, phosphorus, sulfur or oxygen, and the like. A heterocycle comprising more than one ring may be fused, spiro or bridged, or any combination thereof. In fused ring systems, one or more may be fused rings can be cycloalkyl. Particular heterocyclyl groups are 3- to 14-membered rings having 1 to 13 annular carbon atoms and 1 to 6 annular heteroatoms independently selected from nitrogen, phosphorus, oxygen and sulfur; 3- to 12-membered rings having 1 to 11 annular carbon atoms and 1 to 6 annular heteroatoms independently selected from nitrogen, phosphorus, oxygen and sulfur; 3- to 10-membered rings having 1 to 9 annular carbon atoms and 1 to 4 annular heteroatoms independently selected from nitrogen, phosphorus, oxygen and sulfur; 3- to 8-membered rings having 1 to 7 annular carbon atoms and 1 to 4 annular heteroatoms independently selected from nitrogen, phosphorus, oxygen and sulfur; and 3- to 6-membered rings having 1 to 5 annular carbon atoms and 1 to 4 annular heteroatoms independently selected from nitrogen, phosphorus, oxygen and sulfur. In one variation, heterocyclyl include monocyclic 3-, 4-, 5-, 6- or 7-membered rings having from 1 to 2, 1 to 3, 1 to 4, 1 to 5 or 1 to 6 annular carbon atoms and 1 to 2, 1 to 3 or 1 to 4 annular heteroatoms independently selected from nitrogen, phosphorus, oxygen and sulfur. In another variation, heterocyclyl includes polycyclic non-aromatic rings having from 1 to 12 annular carbon atoms and 1 to 6 annular heteroatoms independently selected from nitrogen, phosphorus, oxygen and sulfur.

“Halo” or Halogen” refers to fluoro, chloro, bromo and/or iodo. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halo; thus 4-chloro-3-fluorophenyl is within the scope of dihaloaryl. An alkyl group in which one or more hydrogen is replaced with a halo group is referred to as a “haloalkyl”, for example, “C₁₋₆ haloalkyl.” An alkyl group in which each hydrogen is replaced with a halo group is referred to as a “perhaloalkyl.” A preferred perhaloalkyl group is trifluoroalkyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Carbonyl” refers to the group C═O.

“Oxo” refers to the moiety═O.

“Geminal” refers to the relationship between two moieties that are attached to the same atom. For example, in the residue —CH₂—CR^{x}R^{y}—, R^{x} and R^{y} are geminal and RX may be referred to as a geminal R group to R^{y}.

“Optionally substituted” unless otherwise specified means that a group may be unsubstituted or substituted by one or more (e.g., 1, 2, 3, 4 or 5) of the substituents listed for that group in which the substituents may be the same or different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4 or 1 to 5 substituents.

Use of the word “inhibitor” herein is meant to mean a molecule that inhibits activity of a molecular target (e.g., LATS1/2). By “inhibit” herein is meant to decrease the activity of the target enzyme, as compared to the activity of that enzyme in the absence of the inhibitor. In some embodiments, the term “inhibit” means a decrease in the target enzyme activity of at least about 5%, at least about 10%, at least about 20%, at least about 25%, at least about 50%, at least about 60%, at least about 70%, at least about 80%, at least about 90%, or at least about 95%. In other embodiments, inhibit means a decrease in the target enzyme activity of about 5% to about 25%, about 25% to about 50%, about 50% to about 75%, or about 75% to 100%. In some embodiments, inhibit means a decrease in the target enzyme activity of about 95% to 100%, e.g., a decrease in activity of 95%, 96%, 97%, 98%, 99%, or 100%. Such decreases can be measured using a variety of techniques that would be recognizable by one of skill in the art, including in vitro kinase assays.

As used herein, “treatment” or “treating” in reference to a disease or condition refers to obtaining a desired pharmacologic and/or physiologic effect. The effect may be therapeutic in terms of a partial or complete cure for a disease or condition and/or adverse effect attributable to the disease or condition. “Treatment” as used herein includes, but is not limited to, one or more of the following: decreasing one or more symptoms resulting from the disease or condition, diminishing the extent of the disease or condition, stabilizing the disease or condition (e.g., preventing or delaying the worsening of the disease or condition), delaying or slowing the progression of the disease or condition, ameliorating the disease state, decreasing the dose of one or more medications required to treat the disease or condition, enhancing effect of another medication, increasing the quality of life, interfering with one or more points in the biological pathway that leads to or is responsible for the disease or condition, and/or prolonging survival. Also encompassed by “treatment” is a reduction of pathological consequence of tissue injury and promotion of regeration of an injured tissue. The methods of the invention contemplate any one or more of these aspects of treatment.

As used herein, the term “effective amount” intends such amount of a compound of the invention which in combination with its parameters of efficacy and toxicity, should be effective in a given therapeutic form. As is understood in the art, an effective amount may be in one or more doses, i.e., a single dose or multiple doses may be required to achieve the desired treatment endpoint. An effective amount may be considered in the context of administering one or more therapeutic agents, and a compound, or pharmaceutically acceptable salt thereof may be considered to be given in an effective amount if, in conjunction with one or more other agents, a desirable or beneficial results may be or is achieved. Suitable doses of any of the co-administered compounds may optionally be lowered due to the combined action (e.g., additive or synergistic effects) of the compounds.

A “therapeutically effective amount” refers to an amount of a compound or salt thereof sufficient to produce a desired therapeutic outcome (e.g., reducing the severity or duration of, stabilizing the severity of, or eliminating one or more symptoms of a disease or condition mediated by LATS1/2 (e.g., ARDS). For therapeutic use, beneficial or desired results include, e.g., decreasing one or more symptoms resulting from the disease (biochemical, histologic and/or behavioral), including its complications and intermediate pathological phenotypes presenting during development of the disease, increasing the quality of life of those suffering from the disease, decreasing the dose of other medications required to treat the disease, enhancing effect of another medication, delaying the progression of the disease, and/or prolonging survival of patients.

As used herein, by “pharmaceutically acceptable’ or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug Administration.

In some embodiments, the salts of the compounds of the invention are pharmaceutically acceptable salts. “Pharmaceutically acceptable salts” are those salts which retain at least some of the biological activity of the free (non-salt) compound and which can be administered as drugs or pharmaceuticals to a subject. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound of the invention in its free acid or base form with a suitable organic or inorganic base or acid respectively, and isolating the salt thus formed during subsequent purification.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include e.g. calcium carbonate, dextrose, fructose dc (dc —“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g. dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc. In some cases, the terms “excipient” and “carrier” are used interchangeably.

The term “subject” or “patient” refers to animals such as mammals, including, but not limited to, primates (e.g., humans), cows, sheep, goats, horses, dogs, cats, rabbits, rats, mice and the like. In certain embodiments, the subject is a human or a human patient.

### Compounds

The compounds disclosed herein are compounds of Formula (I), or salts (e.g., pharmaceutically acceptable salts), solvates (e.g., hydrates), prodrugs, metabolites, or derivatives thereof. These compounds bind to and inhibit the activity of LATS1/2 with high potency and selectivity over other kinases (such as AKT1, ROCK1 and PKA), thus are useful as selective inhibitors of LATS1/2 for the treatment of diseases and conditions that can benefit from LATS1/2 inhibition.

In one aspect, provided is a compound of Formula (I):

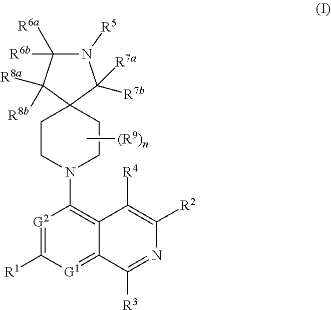

or an N-oxide thereof, or a salt (e.g., a pharmaceutically acceptable salt), solvate (e.g., hydrate), prodrug, metabolite or derivative thereof, wherein:
R¹ is 5- to 14-membered heteroaryl optionally substituted with 1, 2, 3, 4 or 5 substituents independently selected from R¹⁰;
R² is hydrogen, halogen, C₁₋₆ alkyl, —O(C₁₋₆ alkyl), —NH(C₁₋₆ alkyl) or —N(C₁₋₆ alkyl)₂, wherein each C₁₋₆ alkyl is optionally substituted with 1, 2, 3, 4 or 5 substituents independently selected from R¹⁰;
R³ is hydrogen, C₁₋₆ alkyl, halogen, cyano, hydroxyl, —O(C₁₋₆ alkyl), C₂₋₆ alkenyl or C₂₋₆ alkynyl, wherein the C₁₋₆ alkyl, C₂₋₆ alkenyl and C₂₋₆ alkynyl are each optionally substituted with 1, 2, 3, 4 or 5 substituents independently selected from R¹⁰;
R⁴ is hydrogen, halogen, cyano, —NR^{43a}R^{43b}, —OR⁴⁴, C₁₋₆ alkyl or C₃₋₆ cycloalkyl, wherein the C₁₋₆ alkyl and C₃₋₆ cycloalkyl are each optionally substituted with 1, 2, 3, 4 or 5 substituents independently selected from R¹⁰;
G¹ is N or CR⁴¹, G² is N or CR⁴², provided that one or both of G¹ and G² are N;
R⁴¹ and R⁴² are independently hydrogen, halogen, cyano, —NR^{43a}R^{43b}, —OR⁴⁴, C₁₋₆ alkyl or C₃₋₆ cycloalkyl, wherein the C₁₋₆ alkyl and C₃₋₆ cycloalkyl are each optionally substituted with 1, 2, 3, 4 or 5 substituents independently selected from R¹⁰;
each R^{43a} and R^{43b} is independently hydrogen or C₁₋₆ alkyl;
R⁴⁴ is hydrogen, C₁₋₆ alkyl, C₂₋₆ alkenyl, C₂₋₆ alkynyl, C₃₋₈ cycloalkyl, 3- to 14-membered heterocyclyl, wherein the C₁₋₆ alkyl, C₂₋₆ alkenyl, C₂₋₆ alkynyl, C₃₋₈ cycloalkyl and 3- to 14-membered heterocyclyl of R⁴⁴ are each optionally substituted with 1, 2, 3 or 4 substituents independently selected from R¹⁰;
R⁵ is hydrogen, C₁₋₆ alkyl, C₂₋₆ alkenyl, C₂₋₆ alkynyl, C₃₋₈ cycloalkyl, C₆₋₁₄ aryl, 5- to 14-membered heteroaryl, 3- to 14-membered heterocyclyl, —C(O)R¹⁴, —C(O)OR¹⁵ or —C(O)NR^{16a}R^{16b}, wherein the C₁₋₆ alkyl, C₂₋₆ alkenyl, C₂₋₆ alkynyl, C₃₋₈ cycloalkyl, C₆₋₁₄ aryl, 5- to 14-membered heteroaryl and 3- to 14-membered heterocyclyl of R⁵ are each optionally substituted with 1, 2, 3, 4 or 5 substituents independently selected from R¹⁰; or is taken together with R^{6a} or R^{6b} and the atoms to which they are attached to form a 3- to 14-membered heterocyclyl optionally substituted with 1, 2, 3, 4 or 5 substituents independently selected from R¹⁰;
each R^{6a} and R^{6b} is independently hydrogen, C₁₋₆ alkyl, C₃₋₈ cycloalkyl, C₆₋₁₀ aryl, 5- to 14-membered heteroaryl, 3- to 12-membered heterocyclyl, —C(O)R¹⁴, —C(O)OR¹⁵ or —C(O)NR^{16a}R^{16b}, wherein the C₁₋₆ alkyl, C₃₋₈ cycloalkyl, C₆₋₁₀ aryl, 5- to 14-membered heteroaryl, and 3- to 12-membered heterocyclyl of R^{6a} and R^{6b} are each optionally substituted with 1, 2, 3, 4 or 5 substituents independently selected from R¹⁰; or taken together with R⁵ and the atoms to which they are attached to form a 3- to 14-membered heterocyclyl optionally substituted with 1, 2, 3, 4 or 5 substituents independently selected from R¹⁰; or R^{6a} and R^{6b} are taken together with the carbon to which they are attached to form a carbonyl;
each R^{7a} and R^{7b} is independently hydrogen or C₁₋₆ alkyl optionally substituted with 1, 2, 3, 4 or 5 substituents independently selected from R¹⁰; or R^{7a} and R^{7b} are taken together with the carbon to which they are attached to form a carbonyl;
each R^{8a} and R^{8b} is independently hydrogen, halogen, hydroxyl, —O(C₁₋₆ alkyl) or C₁₋₆ alkyl, wherein each C₁₋₆ alkyl is optionally substituted with 1, 2, 3, 4 or 5 substituents independently selected from R¹⁰;
n is 0 to 8;
each R⁹ is independently C₁₋₆ alkyl; or two geminal R^{g} groups, if present, are taken together with the carbon to which they are attached to form a carbonyl;
each R¹⁴ is independently hydrogen or C₁₋₆ alkyl;
each R¹⁵ is independently C₁₋₆ alkyl;
each R^{16a} and R^{16b} is independently hydrogen or C₁₋₆ alkyl; or R^{16a} and R^{16b} are taken together with the nitrogen atom to which they are attached to form a 4- to 12-membered heterocyclyl optionally substituted with 1, 2, 3, 4 or 5 substituents independently selected from R¹⁰;
each R¹⁰ is independently oxo, C₁₋₆ alkyl, C₂₋₆ alkenyl, C₂₋₆ alkynyl, C₃₋₈ cycloalkyl, C₆₋₁₄ aryl, 5- to 14-membered heteroaryl, 3- to 14-membered heterocyclyl, halogen, cyano, —C(O)R^{a}, —C(O)OR^{b}, —C(O)NR^{c}R^{d}, —OR^{b}, —OC(O)R^{a}, —OC(O)NR^{c}R^{d}, —SR^{b}, —S(O)R^{c}, —S(O)₂R^{c}, —S(O)(═NH)R^{c}, —S(O)₂NR^{c}R^{d}, —NR^{c}R^{d}, —N(Rf)C(O)R^{a}, —N(Rf)C(O)OR^{b}, —N(Rf)C(O)NR^{c}R^{d}, —N(R^{f})S(O)₂Rc, —N(R^{f})S(O)₂NR^{c}R^{d} or —P(O)R^{g}R^{h}, wherein the C₁₋₆ alkyl, C₂₋₆ alkenyl, C₂₋₆ alkynyl, C₃₋₈ cycloalkyl, C₆₋₁₄ aryl, 5- to 14-membered heteroaryl and 3- to 14-membered heterocyclyl of R¹⁰ are each optionally substituted with 1, 2, 3 or 4 substituents independently selected from R¹¹;
each R^{a} is independently hydrogen, C₁₋₆ alkyl, C₂₋₆ alkenyl, C₂₋₆ alkynyl, C₃₋₈ cycloalkyl, C₆₋₁₀ aryl, 5- to 10-membered heteroaryl or 3- to 12-membered heterocyclyl, wherein the C₁₋₆ alkyl, C₂₋₆ alkenyl, C₂₋₆ alkynyl, C₃₋₈ cycloalkyl, C₆₋₁₀ aryl, 5- to 10-membered heteroaryl and 3- to 12-membered heterocyclyl of R^{a} are each optionally substituted with 1, 2, 3 or 4 substituents independently selected from R¹¹;
each R^{b} is independently hydrogen, C₁₋₆ alkyl, C₃₋₈ cycloalkyl, C₆₋₁₀ aryl, 5- to 10-membered heteroaryl or 3- to 12-membered heterocyclyl, wherein the C₁₋₆ alkyl, C₃₋₈ cycloalkyl, C₆₋₁₀ aryl, 5- to 10-membered heteroaryl and 3- to 12-membered heterocyclyl of R^{b} are each optionally substituted with 1, 2, 3 or 4 substituents independently selected from R¹¹;
each R^{c} and R^{d} is independently hydrogen, C₁₋₆ alkyl, C₃₋₈ cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{c} and R^{d} are each optionally substituted with 1, 2, 3 or 4 substituents independently selected from R¹¹;
   or R^{c} and R^{d} are taken together with the nitrogen atom to which they are attached to form a 4- to 12-membered heterocyclyl optionally substituted with 1, 2, 3 or 4 substituents independently selected from R¹¹;
each R^{c} is independently C₁₋₆ alkyl, C₃₋₈ cycloalkyl, C₆₋₁₀ aryl, 5- to 10-membered heteroaryl or 3- to 12-membered heterocyclyl, wherein the C₁₋₆ alkyl, C₃₋₈ cycloalkyl, C₆₋₁₀ aryl, 5- to 10-membered heteroaryl and 3- to 12-membered heterocyclyl of Rc are each optionally substituted with 1, 2, 3 or 4 substituents independently selected from R¹¹;
each R^{f2} is independently hydrogen or C₁₋₆ alkyl;
each R^{g} and R^{h} is independently C₁₋₆ alkyl, C₃₋₈ cycloalkyl, C₆₋₁₀ aryl, 5- to 10-membered heteroaryl, 3- to 12-membered heterocyclyl or —O—C₁₋₆ alkyl; wherein the C₁₋₆ alkyl, C₃₋₈ cycloalkyl, C₆₋₁₀ aryl, 5- to 10-membered heteroaryl and 3- to 12-membered heterocyclyl of R^{g} and R^{h} are each optionally substituted with 1, 2, 3 or 4 substituents independently selected from R¹¹;
   or R^{g} and R^{h} are taken together with the phosphorus atom to which they are attached to form a 4- to 12-membered heterocyclyl optionally substituted with 1, 2, 3 or 4 substituents independently selected from R¹¹;
each R¹¹ is independently oxo, C₁₋₆ alkyl, C₂₋₆ alkenyl, C₂₋₆ alkynyl, C₃₋₆ cycloalkyl, C₆₋₁₀ aryl, 5- to 10-membered heteroaryl, 3- to 8-membered heterocyclyl, halogen, cyano, —C(O)R^{a1}, —C(O)OR^{b1}, —C(O)NR^{c1}R^{d1}, —OR¹¹, —OC(O)R^{a1}, —OC(O)NR^{c1}R^{d1}, —SR¹¹, —S(O)R^{c1}, —S(O)₂R^{c1}, —S(O)₂NR^{c1}R^{d1}, —NR^{c1}R^{d1}, —N(R^{f1})C(O)R^{a1}, —N(R¹¹)C(O)OR^{b1}, —N(R¹¹)C(O)NR^{c1}R^{d1}, —N(R^{f1})S(O)₂R^{c1}, —N(R^{f1})S(O)₂NR^{c1}R^{d1} or —P(O)R^{g1}R^{h1}; wherein the C₁₋₆ alkyl, C₂₋₆ alkenyl, C₂₋₆ alkynyl, C₃₋₆ cycloalkyl, C₆₋₁₀ aryl, 5- to 10-membered heteroaryl and 3- to 8-membered heterocyclyl of R¹¹ are each optionally substituted with 1, 2, 3 or 4 substituents independently selected from R¹²;
each R^{a1} is independently hydrogen, C₁₋₆ alkyl, C₂₋₆ alkenyl, C₂₋₆ alkynyl, C₃₋₆ cycloalkyl, C₆₋₁₀ aryl, 5- to 10-membered heteroaryl or 3- to 8-membered heterocyclyl; wherein the C₁₋₆ alkyl, C₂₋₆ alkenyl, C₂₋₆ alkynyl, C₃₋₆ cycloalkyl, C₆₋₁₀ aryl, 5- to 10-membered heteroaryl and 3- to 8-membered heterocyclyl of R¹ are each optionally substituted with 1, 2, 3 or 4 substituents independently selected from R¹²;
each R^{b1} is independently hydrogen, C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{b1} are each optionally substituted with 1, 2, 3 or 4 substituents independently selected from R¹²;
each R^{c1} and R^{d1} is independently hydrogen, C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{c1} and R^{d1} are each optionally substituted with 1, 2, 3 or 4 substituents independently selected from R¹²;
   or R^{c1} and R^{d1} are taken together with the nitrogen atom to which they are attached to form a 4- to 8-membered heterocyclyl optionally substituted with 1, 2, 3 or 4 substituents independently selected from R¹²;
each R^{c1} is independently C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{c1} are each optionally substituted with 1, 2, 3 or 4 substituents independently selected from R¹²;
each R^{f1} is independently hydrogen or C₁₋₆ alkyl;
each R^{g1} and R^{h1} is independently C₁₋₆ alkyl, C₃₋₆ cycloalkyl, C₆₋₁₀ aryl, 5- to 10-membered heteroaryl, 3- to 8-membered heterocyclyl, or —O—C₁₋₆ alkyl; wherein the C₁₋₆ alkyl, C₃₋₆ cycloalkyl, C₆₋₁₀ aryl, 5- to 10-membered heteroaryl and 3- to 8-membered heterocyclyl of R^{g1} and R^{h1} are each optionally substituted with 1, 2, 3 or 4 substituents independently selected from R¹²;
   or R^{g1} and R^{h1} are taken together with the phosphorus atom to which they are attached to form a 4- to 8-membered heterocyclyl optionally substituted with 1, 2, 3 or 4 substituents independently selected from R¹²;
each R¹² is independently oxo, C₁₋₆ alkyl, C₃₋₆ cycloalkyl, C₆ aryl, 5- to 6-membered heteroaryl, 3- to 6-membered heterocyclyl, halogen, cyano, —C(O)R^{a2}, —C(O)OR^{b2}, —C(O)NR^{c2}R^{d2}, —OR^{b2}, —OC(O)R^{a2}, —OC(O)NR^{c2}R^{d2}, —S(O)₂R^{c2}, —S(O)₂NR^{c2}R^{d2}, —NR^{c2}R^{d2}, —N(R^{f2})C(O)R^{a2}, —N(R^{f})C(O)OR^{b2}, —N(R^{f})C(O)NR^{c2}R^{d2}, —N(R^{f})S(O)₂R^{c2}, —N(R^{f2})S(O)₂NR^{c2}R^{d2} or —P(O)R^{g2}R^{h2}; wherein the C₁₋₆ alkyl, C₃₋₆ cycloalkyl, C₆ aryl, 5- to 6-membered heteroaryl and 3- to 6-membered heterocyclyl of R¹² are each optionally substituted with 1, 2, 3 or 4 substituents independently selected from R¹³;
each R^{a2} is independently hydrogen, C₁₋₆ alkyl, C₃₋₆ cycloalkyl, C₆ aryl, 5- to 6-membered heteroaryl or 3- to 6-membered heterocyclyl; wherein the C₁₋₆ alkyl, C₃₋₆ cycloalkyl, C₆ aryl, 5- to 6-membered heteroaryl and 3- to 6-membered heterocyclyl of R^{a2} are each optionally substituted with 1, 2, 3 or 4 substituents independently selected from R¹³;
each R^{b2} is independently hydrogen, C₁₋₆ alkyl, C₃₋₆ cycloalkyl or 3- to 6-membered heterocyclyl; wherein the C₁₋₆ alkyl, C₃₋₆ cycloalkyl and 3- to 6-membered heterocyclyl of R^{b2} are each optionally substituted with 1, 2, 3 or 4 substituents independently selected from R¹³;
each R^{c2} and R^{d2} is independently hydrogen, C₁₋₆ alkyl, C₃₋₆ cycloalkyl or 3- to 8-membered heterocyclyl; wherein the C₁₋₆ alkyl, C₃₋₆ cycloalkyl and 3- to 8-membered heterocyclyl of R^{c2} and R^{d2} are each optionally substituted with 1, 2, 3 or 4 substituents independently selected from R¹³;
   or R^{c2} and R^{d2} are taken together with the nitrogen atom to which they are attached to form a 4- to 6-membered heterocyclyl optionally substituted with 1, 2, 3 or 4 substituents independently selected from R¹³;
each R^{c2} is independently C₁₋₆ alkyl, C₃₋₆ cycloalkyl, C₆ aryl, 5- to 6-membered heteroaryl or 3- to 6-membered heterocyclyl; wherein the C₁₋₆ alkyl, C₃₋₆ cycloalkyl, C₆ aryl, 5- to 6-membered heteroaryl and 3- to 6-membered heterocyclyl of R^{c2} are each optionally substituted with 1, 2, 3 or 4 substituents independently selected from R¹³;
each RV is independently hydrogen or C₁₋₆ alkyl;
each R^{g2} and R^{h2} is independently C₁₋₆ alkyl, C₃₋₆ cycloalkyl, 3- to 8-membered heterocyclyl, or —O—C₁₋₆ alkyl; wherein the C₁₋₆ alkyl, C₃₋₆ cycloalkyl, and 3- to 8-membered heterocyclyl of R^{g2} and R^{h2} are each optionally substituted with 1, 2, 3 or 4 substituents independently selected from R¹³;
   or R^{g2} and R^{h2} are taken together with the phosphorus atom to which they are attached to form a 4- to 6-membered heterocyclyl optionally substituted with 1, 2, 3 or 4 substituents independently selected from R¹³; and
each R¹³ is independently oxo, halogen, hydroxyl, —O(C₁₋₆ alkyl), cyano, C₁₋₆ alkyl or C₁₋₆ haloalkyl.

In one aspect, provided is a compound of Formula (I):

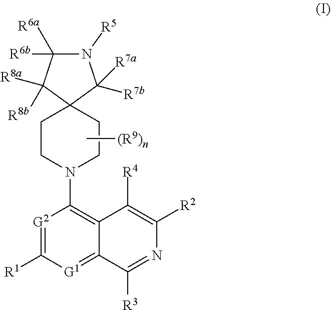

or a salt (e.g., a pharmaceutically acceptable salt), solvate (e.g., hydrate), prodrug, metabolite or derivative thereof, wherein:
R¹ is 5- to 14-membered heteroaryl optionally substituted with 1, 2, 3, 4 or 5 substituents independently selected from R¹⁰;
R² is hydrogen, C₁₋₆ alkyl or —O(C₁₋₆ alkyl), wherein each C₁₋₆ alkyl is optionally substituted with 1, 2, 3, 4 or 5 substituents independently selected from R¹⁰;
R³ is hydrogen, C₁₋₆ alkyl or —O(C₁₋₆ alkyl), wherein each C₁₋₆ alkyl is optionally substituted with 1, 2, 3, 4 or 5 substituents independently selected from R¹⁰;
R⁴ is hydrogen, halogen, cyano, —O(C₁₋₆ alkyl), C₁₋₆ alkyl or C₃₋₆ cycloalkyl, wherein the C₁₋₆ alkyl and C₃₋₆ cycloalkyl are each optionally substituted with 1, 2, 3, 4 or 5 substituents independently selected from R¹⁰;
G¹ is N or CR⁴¹ and G² is N or CR⁴², provided that one or both of G¹ and G² are N;
R⁴¹ and R⁴² are independently hydrogen, halogen, cyano, —O(C₁₋₆ alkyl), C₁₋₆ alkyl or C₃₋₆ cycloalkyl, wherein the C₁₋₆ alkyl and C₃₋₆ cycloalkyl are each optionally substituted with 1, 2, 3, 4 or 5 substituents independently selected from R¹⁰;
R⁵ is hydrogen, C₁₋₆ alkyl, C₃₋₈ cycloalkyl, C₆₋₁₄ aryl, 5- to 14-membered heteroaryl, 3- to 14-membered heterocyclyl, —C(O)R¹⁴, —C(O)OR¹⁵ or —C(O)NR^{16a}R^{16b}, wherein the C₁₋₆ alkyl, C₃₋₈ cycloalkyl, C₆₋₁₄ aryl, 5- to 14-membered heteroaryl and 3- to 14-membered heterocyclyl of R⁵ are each optionally substituted with 1, 2, 3, 4 or 5 substituents independently selected from R¹⁰;
each R^{6a} and R^{6b} is independently hydrogen, C₁₋₆ alkyl, C₃₋₈ cycloalkyl, C₆₋₁₀ aryl, 5- to 14-membered heteroaryl, 3- to 12-membered heterocyclyl, —C(O)R¹⁴, —C(O)OR¹⁵ or —C(O)NR^{16a}R^{16b}, wherein the C₁₋₆ alkyl, C₃₋₈ cycloalkyl, C₆₋₁₀ aryl, 5- to 14-membered heteroaryl, and 3- to 12-membered heterocyclyl of R^{6a} and R^{6b} are each optionally substituted with 1, 2, 3, 4 or 5 substituents independently selected from R¹⁰; or R^{6a} and R^{6b} are taken together with the carbon to which they are attached to form a carbonyl;
each R^{7a} and R^{7b} is independently hydrogen or C₁₋₆ alkyl optionally substituted with 1, 2, 3, 4 or 5 substituents independently selected from R¹⁰; or R^{7a} and R^{7b} are taken together with the carbon to which they are attached to form a carbonyl;
each R^{8a} and R^{8b} is independently hydrogen, halogen, hydroxyl, —O(C₁₋₆ alkyl) or C₁₋₆ alkyl, wherein each C₁₋₆ alkyl is optionally substituted with 1, 2, 3, 4 or 5 substituents independently selected from R¹⁰;
n is 0 to 8;
each R⁹ is independently C₁₋₆ alkyl; or two geminal R^{g} groups, if present, are taken together with the carbon to which they are attached to form a carbonyl;
each R¹⁴ is independently hydrogen or C₁₋₆ alkyl;
each R¹⁵ is independently C₁₋₆ alkyl;
each R^{16a} and R^{16b} is independently hydrogen or C₁₋₆ alkyl; or R^{16a} and R^{16b} are taken together with the nitrogen atom to which they are attached to form a 4- to 12-membered heterocyclyl optionally substituted with 1, 2, 3, 4 or 5 substituents independently selected from R¹⁰; and
each R¹⁰ is as defined herein.

In some embodiments, the compound is other than a compound in Table 1X and salts thereof. In some embodiments, the compound herein, such as a compound of Formula (I), is other than a compound selected from one or more of Compound Nos. 1x-3x in Table 1X. In some embodiments, the compounds of the disclosure, and methods of using the compounds detailed herein, encompass any of the compounds of Formula (I), including those listed in Table 1X and salts thereof.

TABLE 1X

| No. | Name |
|---|---|
| 1x | 2,8-Diazaspiro[4.5]decan-1-one, 8-[3-[2-[(1-methyl-1H-pyrrol-3- |
| | yl)amino]-4-pyridinyl]-2,6-naphthyridin-1-yl]- |
| 2x | 2,8-Diazaspiro[4.5]decan-1-one, 8-[3-(2-chloro-4-pyridinyl)-2,6- |
| | naphthyridin-1-yl]- |
| 3x | 2,8-Diazaspiro[4.5]decan-1-one, 8-[3-[2-[(1-methyl-1H-pyrazol-3- |
| | yl)amino]-4-pyridinyl]-2,6-naphthyridin-1-yl]- |

In some embodiments, the compound is of the Formula (I), or a salt (e.g., a pharmaceutically acceptable salt), solvate (e.g., hydrate), prodrug, metabolites or derivative thereof, wherein (i) both G¹ and G² are N, (ii) G¹ is N and G² is CR⁴², or (iii) G¹ is CR⁴¹ and G² is N.

In some embodiments, the compound is of the Formula (I), or a salt (e.g., a pharmaceutically acceptable salt), solvate (e.g., hydrate), prodrug, metabolites or derivative thereof, provided when G¹ is CR⁴¹ where R⁴¹ is hydrogen, G² is N, R¹ is 2-substituted-4-pyridinyl and each R², R³ and R⁴ is hydrogen, each R^{7a} and R^{7b} is independently hydrogen or C₁₋₆ alkyl optionally substituted with 1, 2, 3, 4 or 5 substituents independently selected from R¹⁰.

In one aspect, provided is a compound of Formula (IA):

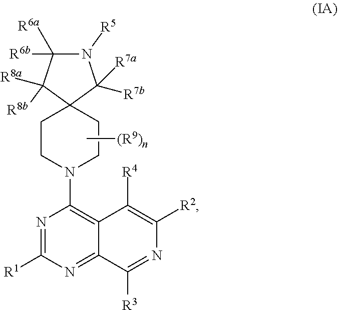

or a salt (e.g., a pharmaceutically acceptable salt), solvate (e.g., hydrate), prodrug, metabolites or derivative thereof, wherein R¹, R², R³, R⁴, R⁵, R^{6a}, R^{6b}, R^{7a}, R^{7b}, R^{8a}, R^{8b}, R⁹ and n are as defined for Formula (I), or variations detailed herein.

In one aspect, provided is a compound of Formula (IB):

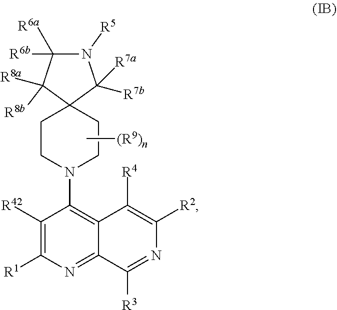

or a salt (e.g., a pharmaceutically acceptable salt), solvate (e.g., hydrate), prodrug, metabolites or derivative thereof, wherein R¹, R², R³, R⁴, R⁴², R⁵, R^{6a}, R^{6b}, R^{7a}, R⁷, R^{8a}, R^{8b}, R⁹ and n are as defined for Formula (I), or variations detailed herein.

In some embodiments, the compound is of the Formula (IB), or a pharmaceutically acceptable salt thereof, wherein R⁴² is hydrogen.

In one aspect, provided is a compound of Formula (IC):

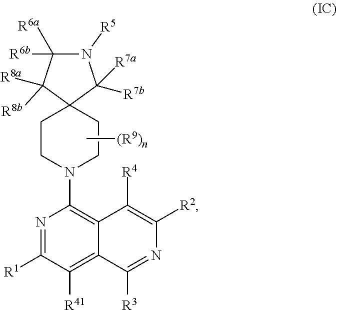

or a salt (e.g., a pharmaceutically acceptable salt), solvate (e.g., hydrate), prodrug, metabolites or derivative thereof, wherein R¹, R², R³, R⁴, R⁴¹, R⁵, R^{6a}, R^{6b}, R^{7a}, R⁷, R^{8a}, R^{8b}, R⁹ and n are as defined for Formula (I), or variations detailed herein.

In some embodiments, the compound is of the Formula (I) or (IC), or a pharmaceutically acceptable salt thereof, provided that the compound is other than a compound selected from one or more of Compound Nos. 1x-3x in Table 1X and salts thereof. In some embodiments, the compound is of the Formula (IC), or a pharmaceutically acceptable salt thereof, wherein R⁴¹ is hydrogen. In some embodiments, R^{7a} and R^{7b} are not taken together with the carbon to which they are attached to form a carbonyl. In some embodiments, R¹ is other than a 2-substituted-4-pyridinyl. In some embodiments, when R¹ is a 2-substituted-4-pyridinyl, each R^{7a} and R^{7b} is independently hydrogen or C₁₋₆ alkyl optionally substituted with 1, 2, 3, 4 or 5 substituents independently selected from R¹⁰.

In some embodiments, the compound is of the Formula (I), or variations thereof such as Formula (IA), (IB) and (IC), or a salt (e.g., a pharmaceutically acceptable salt) thereof, wherein R¹ is a 5- to 14-membered heteroaryl optionally substituted with 1, 2, 3, 4 or 5 substituents independently selected from R¹⁰. In some of these embodiments, R¹ is a 5- to 14-membered heteroaryl having 1 to 12 annular (or ring) carbon atoms and 1 to 6 annular (or ring) heteroatoms independently selected from nitrogen, oxygen and sulfur. In some of these embodiments, R¹ is a 5- to 10-membered heteroaryl having 1 to 8 annular carbon atoms and 1 to 4 annular heteroatoms independently selected from nitrogen, oxygen and sulfur. In some of these embodiments, R¹ is a 5-, 6- or 7-membered heteroaryl having 1 to 5 annular carbon atoms and 1 to 4 annular heteroatoms independently selected from nitrogen, oxygen and sulfur. In some of these embodiments, R¹ is a monocyclic 5-, 6- or 7-membered heteroaryl having from 1 to 6 annular carbon atoms and 1 to 4 annular heteroatoms independently selected from nitrogen, oxygen and sulfur. In some of these embodiments, R¹ is a polycyclic heteroaryl having from 1 to 12 annular carbon atoms and 1 to 6 annular heteroatoms independently selected from nitrogen, oxygen and sulfur.

In some embodiments, R¹ is a monocyclic 5-membered heteroaryl having 1, 2 or 3 ring heteroatoms selected from nitrogen, oxygen and sulfur, optionally substituted with 1, 2, 3 or 4 substituents independently selected from R¹⁰. In some embodiments, R¹ is a monocyclic 5-membered heteroaryl having 1 or 2 ring nitrogen atoms, optionally substituted with 1, 2, 3 or 4 substituents independently selected from R¹⁰. In some embodiments, R¹ is a monocyclic 6-membered heteroaryl having 1 or 2 ring nitrogen atoms, optionally substituted with 1, 2, 3, 4 or 5 substituents independently selected from R¹⁰. In some embodiments, R¹ is a fused bicyclic heteroaryl having 1 to 4 ring heteroatoms selected from nitrogen, oxygen and sulfur, each of which is optionally substituted with 1, 2, 3, 4 or 5 substituents independently selected from R¹⁰. In some embodiments, R¹ is a 5,6-fused bicyclic heteroaryl having 1, 2, 3 or 4 ring nitrogen atoms, optionally substituted with 1, 2, 3, 4 or 5 substituents independently selected from R¹⁰. In some embodiments, R¹ is a 5,6-fused bicyclic heteroaryl having 1 or 2 ring nitrogen atoms, optionally substituted with 1, 2, 3, 4 or 5 substituents independently selected from R¹⁰.

In some embodiments, R¹ is a pyrazolyl, oxazolyl, isoxazolyl, thiazolyl, isothiazolyl or 1,2,4-triazolyl, each of which is optionally substituted with 1 to 3 substituents independently selected from R¹⁰.

In some embodiments, R¹ is a pyrazolyl optionally substituted with 1 to 3 substituents independently selected from R¹⁰. In one variation, R¹ is a pyrazol-3-yl, pyrazol-4-yl or pyrazol-5-yl optionally substituted with 1 to 3 substituents independently selected from R¹⁰. In some embodiments, R¹ is a pyrazol-4-yl optionally substituted with 1 to 3 substituents independently selected from R¹⁰. In some embodiments, R¹ is an isothiazolyl optionally substituted with 1 to 3 substituents independently selected from R¹⁰. In one variation, R¹ is an isothiazol-3-yl, isothiazol-4-yl or isothiazol-5-yl optionally substituted with 1 to 3 substituents independently selected from R¹⁰. In some embodiments, R¹ is an isothiazol-5-yl optionally substituted with 1 to 3 substituents independently selected from R¹⁰. In some of these embodiments, R¹⁰ is selected from halogen (e.g., chloro), cyano and C₁₋₆ alkyl optionally substituted with halogen (e.g., methyl or trifluoromethyl). In some embodiments, R¹ is pyrazol-4-yl optionally substituted with 1 to 3 substituents independently selected from the group consisting of halogen (e.g., chloro), cyano, unsubstituted C₁₋₆ alkyl (e.g., methyl) and C₁₋₆haloalkyl (e.g., trifluoromethyl). In some particular embodiments, R¹ is 3-methylpyrazol-4-yl or 5-methylpyrazol-4-yl. In some particular embodiments, R¹ is 4-methylisothiazol-5-yl.

In some embodiments, R¹ is a pyridyl optionally substituted with 1 to 5 substituents independently selected from R¹⁰. In one variation, R¹ is 4-pyridyl optionally substituted with 1 to 5 substituents independently selected from R¹⁰. In some particular embodiments, R¹ is a 4-pyridyl (also known as pyridin-4-yl).

In some embodiments, R¹ is a pyrimidyl optionally substituted with 1 to 5 substituents independently selected from R¹⁰. In one variation, R¹ is a pyrimid-4-yl optionally substituted with 1 to 5 substituents independently selected from R¹⁰. In some particular embodiments, R¹ is a pyrimid-4-yl.

In some embodiments, R¹ is a 5,6-fused heteroaryl having 1-4 ring nitrogen atoms (e.g., pyrrolo-pyridinyl, indazolyl, imidazo-pyridinyl, pyrrolo-pyrimidinyl, or pyrazolo-pyrimidinyl) optionally substituted with 1 to 5 substituents independently selected from R¹⁰.

In some embodiments, R¹ is a pyrrolo-pyridinyl optionally substituted with 1 to 5 substituents independently selected from R¹⁰. In one variation, R¹ is pyrrolo[2,3-b]pyridinyl (e.g., pyrrolo[2,3-b]pyridin-4-yl) optionally substituted with 1 to 5 substituents independently selected from R¹⁰. In some particular embodiments,

In some embodiments, R¹ is pyrazolyl (e.g., pyrazol-3-yl, pyrazol-4-yl or pyrazol-5-yl), pyridinyl (e.g., 4-pyridyl) or pyrrolo-pyridinyl (e.g., pyrrolo[2,3-b]pyridin-4-yl), each of which is optionally substituted with 1 to 3 substituents independently selected from R¹⁰. In some embodiments, R¹ is pyrazol-4-yl, 4-pyridyl or pyrrolo[2,3-b]pyridin-4-yl, each of which is optionally substituted with 1 to 3 substituents independently selected from the group consisting of halogen (e.g., chloro), cyano, unsubstituted C₁₋₆ alkyl (e.g., methyl) and C₁₋₆ haloalkyl (e.g., trifluoromethyl).

In some embodiments, R¹ is selected from the group consisting of:

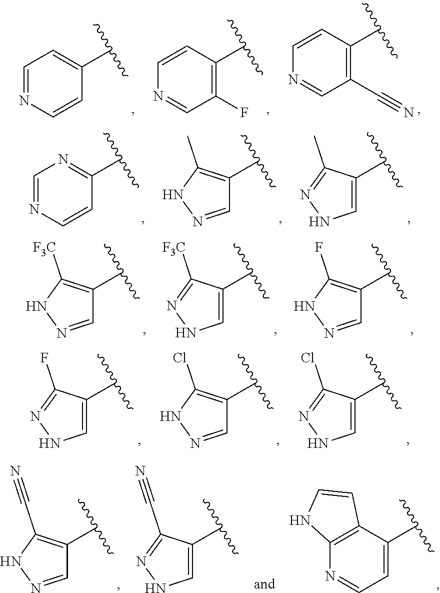

wherein the wavy line in each group indicates the point of attachment to the parent structure. In some embodiments, R¹ is selected from the group consisting of:

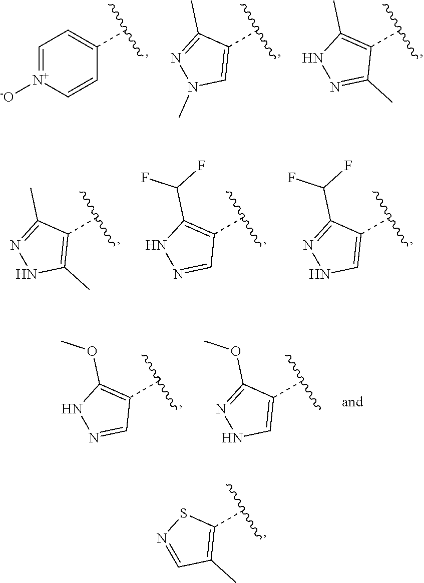

wherein the wavy line in each group indicates the point of attachment to the parent structure.

In some embodiments, the compound is of the Formula (I), or variations thereof such as Formula (IA), (IB) and (IC), or a salt (e.g., a pharmaceutically acceptable salt) thereof, wherein R² is hydrogen, halogen, C₁₋₆ alkyl, —O(C₁₋₆ alkyl), —NH(C₁₋₆ alkyl) or —N(C₁₋₆ alkyl)₂, wherein each C₁₋₆ alkyl is optionally substituted with 1, 2, 3, 4 or 5 substituents independently selected from R¹⁰. In some embodiments, R² is hydrogen, C₁₋₆ alkyl or —O(C₁₋₆ alkyl), wherein each C₁₋₆ alkyl is optionally substituted with 1, 2, 3, 4 or 5 substituents independently selected from R¹⁰. In some embodiments, R² is hydrogen, —NH(C₁₋₆ alkyl), or C₁₋₆ alkyl optionally substituted with 1, 2, 3, 4 or 5 substituents independently selected from R¹⁰. In some embodiments, R² is hydrogen or C₁₋₆ alkyl optionally substituted with 1, 2, 3, 4 or 5 substituents independently selected from R¹⁰. In some embodiments, R² is hydrogen. In some embodiments, R² is hydrogen or C₁₋₆ alkyl (e.g., methyl). In some embodiments, R² is C₁₋₆ alkyl optionally substituted with 1 to 5 substituents independently selected from R¹⁰. In some embodiments, R² is C₁₋₆ alkyl optionally substituted with one or more halogen (e.g., fluoro). In some embodiments, R² is C₁₋₆ alkyl optionally substituted with C₆₋₁₀ aryl (e.g., phenyl) optionally substituted with 1, 2, 3 or 4 substituents independently selected from R¹¹. In some embodiments, R² is C₁₋₆ alkyl optionally substituted with 5- to 10-membered heteroaryl (e.g., pyrazolyl) optionally substituted with 1, 2, 3 or 4 substituents independently selected from R¹¹. In some embodiments, R² is C₁₋₆ alkyl optionally substituted with —N(R^{f})C(O)R^{a}. In some of these embodiments, R^{f2} is hydrogen and R^{a} is C₁₋₆ alkyl. In some embodiments, R² is —NH(C₁₋₆ alkyl), where the C₁₋₆ alkyl optionally substituted with 1, 2, 3, 4 or 5 substituents independently selected from R¹⁰. In some embodiments, R² is —NH(C₁₋₆ alkyl) (e.g, NHMe).

In some embodiments, R² is hydrogen, C₁₋₆ alkyl (e.g., methyl), or C₁₋₆ alkyl substituted with halogen, acylamino, phenyl or pyrazolyl which may be further substituted by halogen (e.g., 2,2,2-trifluoroethyl, —CH₂NHC(O)CH₂CH₃, benzyl and 4-chloropyrazol-1-yl). In some embodiments, R² is hydrogen, —NH(C₁₋₆ alkyl) (e.g, NHMe), C₁₋₆ alkyl (e.g., methyl), or C₁₋₆ alkyl substituted with halogen, acylamino, phenyl or pyrazolyl which may be further substituted by halogen (e.g., 2,2,2-trifluoroethyl, —CH₂NHC(O)CH₂CH₃, benzyl and 4-chloropyrazol-1-yl).

In some embodiments, R² is selected from the group consisting of hydrogen, methyl,

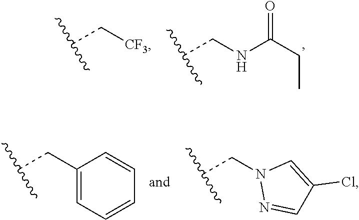

wherein the wavy line in each group indicates the point of attachment to the parent structure. In some embodiments, R² is

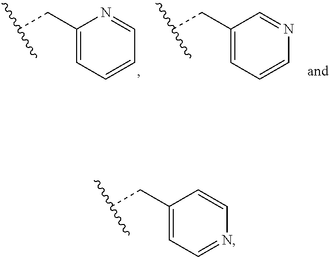

selected from the group consisting of NHMe, CF₃, and N wherein the wavy line in each group indicates the point of attachment to the parent structure.

In some embodiments, the compound is of the Formula (I), or variations thereof such as Formula (IA), (IB) and (IC), or a salt (e.g., a pharmaceutically acceptable salt) thereof, wherein R³ is hydrogen, C₁₋₆ alkyl, halogen, cyano, hydroxyl, —O(C₁₋₆ alkyl), C₂₋₆ alkenyl or C₂₋₆ alkynyl, wherein the C₁₋₆ alkyl, C₂₋₆ alkenyl and C₂₋₆ alkynyl are each optionally substituted with 1, 2, 3, 4 or 5 substituents independently selected from R¹⁰. In some embodiments, R³ is hydrogen, halogen, cyano, hydroxyl, —O(C₁₋₆ alkyl), C₁₋₆ alkyl or C₂₋₆ alkynyl, wherein the C₁₋₆ alkyl and C₂₋₆ alkynyl are each optionally substituted with 1, 2, 3, 4 or 5 substituents independently selected from R¹⁰. In some embodiments, R³ is hydrogen, halogen, C₁₋₆ alkyl or —O(C₁₋₆ alkyl), wherein each C₁₋₆ alkyl is optionally substituted with 1, 2, 3, 4 or 5 substituents independently selected from R¹⁰. In some embodiments, R³ is hydrogen, C₁₋₆ alkyl or —O(C₁₋₆ alkyl), wherein each C₁₋₆ alkyl is optionally substituted with 1, 2, 3, 4 or 5 substituents independently selected from R¹⁰. In some embodiments, R³ is hydrogen or C₁₋₆ alkyl optionally substituted with 1, 2, 3, 4 or 5 substituents independently selected from R¹⁰. In some embodiments, R³ is hydrogen, C₁₋₆ alkyl, or C₁₋₆ haloalkyl. In some embodiments, R³ is hydrogen or C₁₋₆ alkyl. In some embodiments, R³ is hydrogen. In some embodiments, R³ is hydrogen, halogen (e.g., chloro), cyano, hydroxyl or —O(C₁₋₆ alkyl). In some embodiments, R³ is C₁₋₆ alkyl (e.g., methyl). In some embodiments, R³ is C₁₋₆ alkyl optionally substituted by alkoxy (e.g., CH₂OCH₃). In some embodiments, R³ is C₁₋₆haloalkyl (e.g., 2,2,2-trifluoroethyl). In some embodiments, R³—O(C₁₋₆ alkyl), wherein the C₁₋₆ alkyl is optionally substituted with 1, 2, 3, 4 or 5 substituents independently selected from R¹⁰. In some embodiments, R³—O(C₁₋₆ alkyl) (e.g., methoxy). In some embodiments, R³ is C₂₋₆ alkynyl optionally substituted with 1, 2, 3, 4 or 5 substituents independently selected from R¹⁰. In some embodiments, R³ is C₂₋₆ alkynyl optionally substituted with one or more hydroxyl (e.g., 3-hydroxyprop-1-yn-1-yl or 3-hydroxy-3-methylbut-1-yn-1-yl). In some embodiments, R³ is selected from the group consisting of hydrogen, methyl and 2,2,2-trifluoroethyl. In some embodiments, R³ is selected from the group consisting of chloro, cyano, hydroxyl, methoxy, 3-hydroxyprop-1-yn-1-yl, 3-hydroxy-3-methylbut-1-yn-1-yl and methoxymethyl.

In some embodiments, the compound is of the Formula (I), or variations thereof such as Formula (IA), (IB) and (IC), or a salt (e.g., a pharmaceutically acceptable salt) thereof, wherein R⁴ is hydrogen, halogen, cyano, —NR^{43a}R^{43b}, —OR⁴⁴, C₁₋₆ alkyl or C₃₋₆ cycloalkyl, wherein the C₁₋₆ alkyl and C₃₋₆ cycloalkyl are each optionally substituted with 1, 2, 3, 4 or 5 substituents independently selected from R¹⁰; each R^{43a} and R^{43b} is independently hydrogen or C₁₋₆ alkyl; R⁴⁴ is hydrogen, C₁₋₆ alkyl, C₂₋₆ alkenyl, C₂₋₆ alkynyl, C₃₋₈ cycloalkyl, 3- to 14-membered heterocyclyl, wherein the C₁₋₆ alkyl, C₂₋₆ alkenyl, C₂₋₆ alkynyl, C₃₋₈ cycloalkyl and 3- to 14-membered heterocyclyl of R⁴⁴ are each optionally substituted with 1, 2, 3 or 4 substituents independently selected from R¹⁰. In some embodiments, R⁴ is hydrogen, halogen, —NR^{43a}R^{43b}, —OR⁴⁴, C₁₋₆ alkyl or C₃₋₆ cycloalkyl, wherein the C₁₋₆ alkyl and C₃₋₆ cycloalkyl are each optionally substituted with 1, 2, 3, 4 or 5 substituents independently selected from R¹⁰. In some embodiments, R⁴ is hydrogen, halogen, C₁₋₆ alkyl or —O(C₁₋₆ alkyl), wherein each C₁₋₆ alkyl is optionally substituted with 1, 2, 3, 4 or 5 substituents independently selected from R¹⁰. In some embodiments, R⁴ is hydrogen, halogen, cyano, —O(C₁₋₆ alkyl), C₁₋₆ alkyl or C₃₋₆ cycloalkyl, wherein the C₁₋₆ alkyl and C₃₋₆ cycloalkyl are each optionally substituted with 1, 2, 3, 4 or 5 substituents independently selected from R¹⁰. In some embodiments, R⁴ is hydrogen, halogen or C₁₋₆ alkyl optionally substituted with 1, 2, 3, 4 or 5 substituents independently selected from R¹⁰. In some embodiments, R⁴ is C₁₋₆ alkyl optionally substituted with 1, 2, 3, 4 or 5 substituents independently selected from R¹⁰. In some embodiments, R⁴ is —O(C₁₋₆ alkyl) optionally substituted with 1, 2, 3, 4 or 5 substituents independently selected from R¹⁰. In some embodiments, R⁴ is hydrogen, halogen, C₁₋₆ alkyl, or C₃₋₆ cycloalkyl. In some embodiments, R⁴ is hydrogen, halogen or C₁₋₆ alkyl. In some embodiments, R⁴ is hydrogen. In some embodiments, R⁴ is halogen (e.g., fluoro, chloro or bromo). In some embodiments, R⁴ is C₁₋₆ alkyl (e.g., methyl, ethyl, 1-propyl or 2-propyl). In some embodiments, R⁴ is selected from the group consisting of hydrogen, fluoro, chloro, methyl and cyclopropyl.

In some embodiments, R⁴ is —OR⁴⁴, where R⁴⁴ is hydrogen, C₁₋₆ alkyl, C₂₋₆ alkenyl, C₂₋₆ alkynyl, C₃₋₈ cycloalkyl, 3- to 14-membered heterocyclyl, wherein the C₁₋₆ alkyl, C₂₋₆ alkenyl, C₂₋₆ alkynyl, C₃₋₈ cycloalkyl and 3- to 14-membered heterocyclyl of R⁴⁴ are each optionally substituted with 1, 2, 3 or 4 substituents independently selected from R¹⁰. In some of these embodiments, R⁴⁴ is hydrogen. In some of these embodiments, R⁴⁴ is C₁₋₆ alkyl optionally substituted with 1, 2, 3 or 4 substituents independently selected from R¹⁰. In some of these embodiments, R⁴⁴ is C₁₋₆ alkyl optionally substituted with 1, 2, 3 or 4 substituents independently selected from the group consisting of halogen (e.g., fluroro), hydroxyl, alkoxy (e.g., methoxy), 3- to 14-membered heterocyclyl (e.g., oxetanyl), C₂₋₆ alkenyl (e.g., vinyl) and C₂₋₆ alkynyl (e.g., ethynyl). In some of these embodiments, R⁴⁴ is C₂₋₆ alkenyl (e.g., allyl). In some of these embodiments, R⁴⁴ is C₂₋₆ alkynyl optionally substituted with hydroxyl (e.g., 3-hydroxy-3-methylbut-3-yn-1-yl). In some of these embodiments, R⁴⁴ is C₃₋₈ cycloalkyl optionally substituted with 1, 2, 3 or 4 substituents independently selected from R¹⁰. In some of these embodiments, R⁴⁴ is C₃₋₈ cycloalkyl optionally substituted with cyano (e.g., 3-cyanocyclobutyl). In some of these embodiments, R⁴⁴ is 3- to 14-membered heterocyclyl optionally substituted with 1, 2, 3 or 4 substituents independently selected from R¹⁰. In some of these embodiments, R⁴⁴ is 3- to 14-membered heterocyclyl (e.g., oxetan-3-yl). In some of these embodiments, R⁴⁴ is 3- to 14-membered heterocyclyl optionally substituted with acyl (e.g., 1-acetylazetidin-3-yl). In some embodiments, R⁴ is —NR^{43a}R^{43b}, where each R^{43a} and R^{43b} is independently hydrogen or C₁₋₆ alkyl. In some embodiments, R⁴ is —NR^{43a}R^{43b}, where each R^{43a} and R^{43b} is independently C₁₋₆ alkyl (e.g., dimethylamino). In some embodiments, R⁴ is selected from the group consisting

of hydrogen, fluoro, chloro, bromo, methyl,

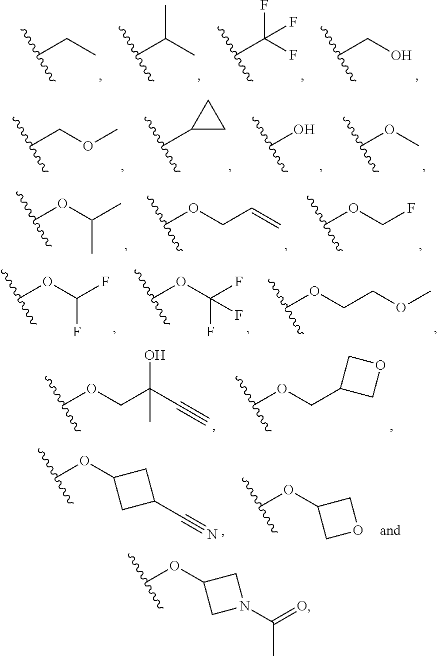

wherein the wavy line in each group indicates the point of attachment to the parent structure.

It is intended and understood that each and every variation of R¹, R², R³ and R⁴ described for the Formula (I), (IA), (IB) or (IC) may be combined, the same as if each and every combination is specifically and individually described. For example, in some embodiments, R¹ is pyrazolyl (e.g., pyrazol-3-yl, pyrazol-4-yl or pyrazol-5-yl), pyridinyl (e.g., 4-pyridyl) or pyrrolo-pyridinyl (e.g., pyrrolo[2,3-b]pyridin-4-yl), each of which is optionally substituted with 1 to 3 substituents independently selected from R¹⁰; R² is hydrogen or C₁₋₆ alkyl (e.g., methyl) optionally substituted with 1 to 5 substituents independently selected from R¹⁰; R³ is hydrogen or C₁₋₆ alkyl (e.g., methyl); and R⁴ is hydrogen, halogen or C₁₋₆ alkyl. In some embodiments, R¹ is pyrazol-4-yl, 4-pyridyl or pyrrolo[2,3-b]pyridin-4-yl, each of which is optionally substituted with 1 to 3 substituents independently selected from the group consisting of halogen (e.g., chloro), cyano, unsubstituted C₁₋₆ alkyl (e.g., methyl) and C₁₋₆ haloalkyl (e.g., trifluoromethyl); each R² and R³ is independently hydrogen or C₁₋₆ alkyl; and R⁴ is hydrogen, halogen (e.g., chloro) or C₁₋₆ alkyl (e.g., methyl). In some embodiments, R¹ is pyrazolyl (e.g., pyrazol-3-yl, pyrazol-4-yl or pyrazol-5-yl), isothiazolyl (e.g., 4-methylisothiazol-5-yl), pyridinyl (e.g., 4-pyridyl) or pyrrolo-pyridinyl (e.g., pyrrolo[2,3-b]pyridin-4-yl), each of which is optionally substituted with 1 to 3 substituents independently selected from R¹⁰; R² is hydrogen or C₁₋₆ alkyl (e.g., methyl) optionally substituted with 1 to 5 substituents independently selected from R¹⁰; R³ is hydrogen, halogen (e.g., chloro) or C₁₋₆ alkyl (e.g., methyl); and R⁴ is hydrogen, halogen, C₁₋₆ alkyl or —O(C₁₋₆ alkyl), wherein each C₁₋₆ alkyl is optionally substituted with 1, 2, 3, 4 or 5 substituents independently selected from R¹⁰. In some embodiments, R¹ is pyrazol-4-yl or 4-pyridyl, each of which is optionally substituted with 1 to 3 substituents independently selected from the group consisting of halogen (e.g., chloro), cyano, unsubstituted C₁₋₆ alkyl (e.g., methyl) and C₁₋₆ haloalkyl (e.g., trifluoromethyl); R² is hydrogen or C₁₋₆ alkyl; R³ is hydrogen, halogen (e.g., chloro) or C₁₋₆ alkyl (e.g., methyl); and R⁴ is hydrogen, halogen (e.g., chloro), C₁₋₆ alkyl (e.g., methyl) or —O(C₁₋₆ alkyl) (e.g., methoxy).

In some embodiments, the compound is of the Formula (I), or variations thereof such as Formula (IA), (IB) and (IC), or a salt (e.g., a pharmaceutically acceptable salt) thereof, wherein R⁵ is hydrogen, C₁₋₆ alkyl, C₂₋₆ alkenyl, C₂₋₆ alkynyl, C₃₋₈ cycloalkyl, C₆₋₁₄ aryl, 5- to 14-membered heteroaryl, 3- to 14-membered heterocyclyl, —C(O)R¹⁴, —C(O)OR¹⁵ or —C(O)NR^{16a}R^{16b}, wherein the C₁₋₆ alkyl, C₂₋₆ alkenyl, C₂₋₆ alkynyl, C₃₋₈ cycloalkyl, C₆₋₁₄ aryl, 5- to 14-membered heteroaryl and 3- to 14-membered heterocyclyl of R⁵ are each optionally substituted with 1, 2, 3, 4 or 5 substituents independently selected from R¹⁰; or is taken together with R^{6a} or R^{6b} and the atoms to which they are attached to form a 3- to 14-membered heterocyclyl optionally substituted with 1, 2, 3, 4 or 5 substituents independently selected from R¹⁰. In some embodiments, R⁵ is hydrogen, C₁₋₆ alkyl, C₃₋₈ cycloalkyl, C₆₋₁₄ aryl, 5- to 14-membered heteroaryl, 3- to 14-membered heterocyclyl, —C(O)R¹⁴, —C(O)OR¹⁵ or —C(O)NR^{16a}R^{16b}, wherein the C₁₋₆ alkyl, C₃₋₈ cycloalkyl, C₆₋₁₄ aryl, 5- to 14-membered heteroaryl and 3- to 14-membered heterocyclyl of R⁵ are each optionally substituted with 1, 2, 3, 4 or 5 substituents independently selected from R¹⁰. In some embodiments, R⁵ is hydrogen, C₁₋₆ alkyl, C₃₋₈ cycloalkyl, C₆₋₁₄ aryl, 5- to 14-membered heteroaryl, 3- to 14-membered heterocyclyl or —C(O)R¹⁴, wherein the C₁₋₆ alkyl, C₃₋₈ cycloalkyl, C₆₋₁₄ aryl, 5- to 14-membered heteroaryl and 3- to 14-membered heterocyclyl of R⁵ are each optionally substituted with 1, 2, 3, 4 or 5 substituents independently selected from R¹⁰. In some embodiments, R⁵ is hydrogen or C₁₋₆ alkyl optionally substituted with 1, 2, 3, 4 or 5 substituents independently selected from R¹⁰. In some embodiments, R⁵ is taken together with R^{6a} or R^{6b} and the atoms to which they are attached to form a 3- to 14-membered heterocyclyl optionally substituted with 1, 2, 3, 4 or 5 substituents independently selected from R¹⁰.

In some embodiments, R⁵ is hydrogen or —C(O)R¹⁴. In some embodiments, R¹⁴ is hydrogen or C₁₋₆ alkyl (e.g., methyl). In some embodiments, R¹⁴ is C₁₋₆ alkyl (e.g., methyl). In some embodiments, R⁵ is hydrogen or acetyl. In some embodiments, R⁵ is hydrogen.

In some embodiments, R⁵ is C₁₋₆ alkyl optionally substituted with 1, 2, 3, 4 or 5 substituents independently selected from R¹⁰. In some embodiments, R⁵ is C₁₋₆ alkyl optionally substituted with 1, 2, 3, 4 or 5 substituents independently selected from R¹⁰, where R¹⁰ is selected from the group consisting of halogen (e.g., fluoro), cyano, —OR^{b}, —N(R^{g}C(O)R^{a}, —N(R^{g}S(O)₂Rc, —S(O)₂NR^{c}R^{d}, —C(O)NR^{c}R^{d}, C₆₋₁₀ aryl optionally substituted with 1, 2, 3 or 4 substituents independently selected from R¹¹, and 3- to 12-membered heterocyclyl optionally substituted with 1, 2, 3 or 4 substituents independently selected from R¹¹. In some of these embodiments, R^{a} is C₁₋₆ alkyl, R^{b} is hydrogen or C₁₋₆ alkyl, R^{c} is C₁₋₆ alkyl and each R^{c}, R^{d} and R^{f2} is hydrogen. In some embodiments, R⁵ is C₁₋₆ alkyl optionally substituted with 1, 2, 3, 4 or 5 substituents independently selected from the group consisting of halogen (e.g., fluoro), cyano, hydroxyl, —O(C₁₋₆ alkyl), —NHC(O)(C₁₋₆ alkyl), —NHS(O)₂(C₁₋₆ alkyl), —S(O)₂NH₂, —C(O)NH₂, phenyl and 3- to 12-membered heterocyclyl (e.g., oxetan-3-yl).

In some embodiments, R⁵ is C₁₋₆ alkyl (e.g., methyl, ethyl, 1-propyl, 2-propyl, 2-selected from the group consisting of:

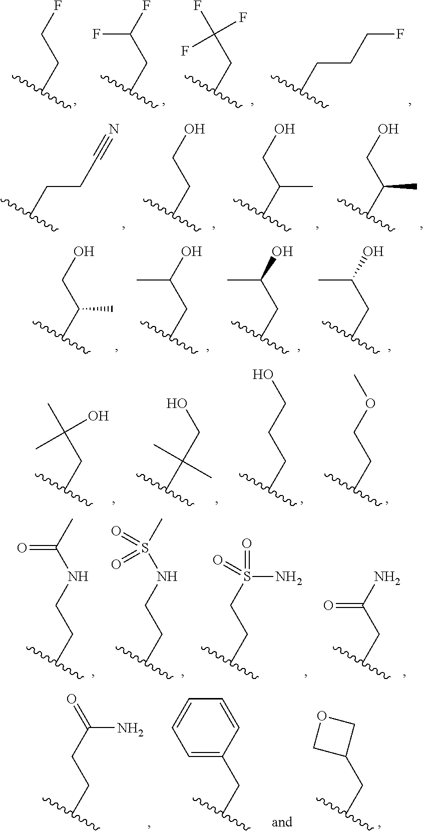

wherein the wavy line in each group indicates the point of attachment to the parent structure. In some embodiments, R⁵ is substituted C₁₋₆ alkyl selected from the group consisting of:

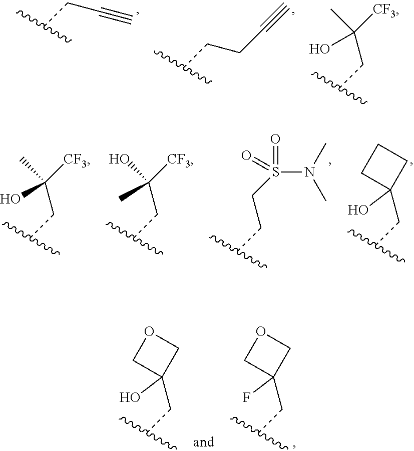

wherein the wavy line in each group indicates the point of attachment to the parent structure.

In some embodiments, R⁵ is C₃₋₈ cycloalkyl optionally substituted with 1, 2, 3, 4 or 5 substituents independently selected from R¹⁰. In some embodiments, R⁵ is C₄₋₈ cycloalkyl optionally substituted with 1, 2, 3, 4 or 5 substituents independently selected from R¹⁰. In some embodiments, R⁵ is C₃₋₆ cycloalkyl optionally substituted with 1, 2, 3, 4 or 5 substituents independently selected from R¹⁰. In some embodiments, R⁵ is C₄₋₆ cycloalkyl optionally substituted with 1, 2, 3, 4 or 5 substituents independently selected from R¹⁰. In some embodiments, R⁵ is C₄₋₈ cycloalkyl optionally substituted with 1, 2, 3, 4 or 5 substituents independently selected from R¹⁰, where R¹⁰ is selected from the group consisting of halogen (e.g., fluoro), cyano and hydroxyl. In some embodiments, R⁵ is C₃₋₆ cycloalkyl. In some embodiments, R⁵ is C₃₋₆ cycloalkyl substituted with 1, 2, 3, 4 or 5 substituents independently selected from the group consisting of halogen (e.g., fluoro), cyano and hydroxyl.

In some embodiments, R⁵ is selected from the group consisting of

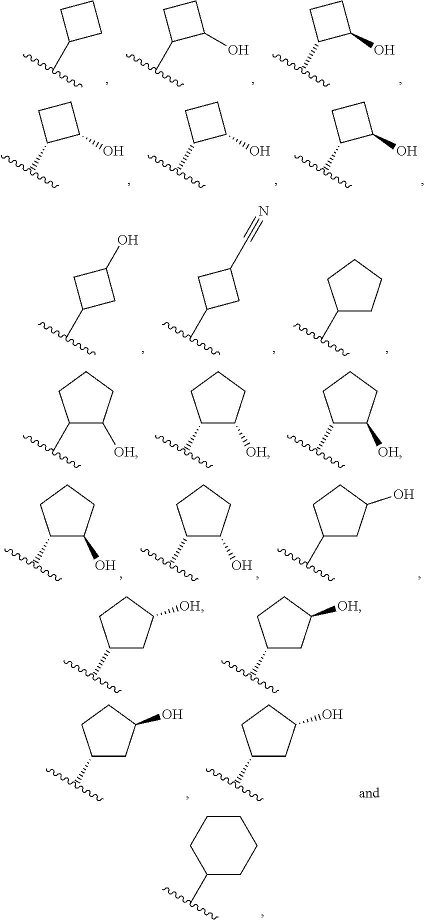

wherein the wavy line in each group indicates the point of attachment to the parent structure.

In some embodiments, R⁵ is 3- to 14-membered heterocyclyl optionally substituted with 1, 2, 3, 4 or 5 substituents independently selected from R¹⁰, C₆₋₁₄ aryl optionally substituted with 1, 2, 3, 4 or 5 substituents independently selected from R¹⁰, or 5- to 14-membered heteroaryl optionally substituted with 1, 2, 3, 4 or 5 substituents independently selected from R¹⁰.

In some embodiments, R⁵ is a 3- to 14-membered heterocyclyl optionally substituted with 1, 2, 3, 4 or 5 substituents independently selected from R¹⁰. In some embodiments, R⁵ is a 3- to 10-membered heterocyclyl optionally substituted with 1, 2, 3, 4 or 5 substituents independently selected from R¹⁰. In some embodiments, R⁵ is a 3- to 10-membered heterocyclyl having 1 to 9 annular carbon atoms and 1 to 4 annular heteroatoms independently selected from nitrogen, oxygen and sulfur, which is optionally substituted with 1, 2, 3, 4 or 5 substituents independently selected from R¹⁰. In some embodiments, R⁵ is monocyclic 3-, 4-, 5-, 6- or 7-membered heterocyclyl having from 1 to 2, 1 to 3, 1 to 4, 1 to 5 or 1 to 6 annular carbon atoms and 1 to 2, 1 to 3 or 1 to 4 annular heteroatoms independently selected from nitrogen, oxygen and sulfur, which is optionally substituted with 1, 2, 3, 4 or 5 substituents independently selected from R¹⁰. In some embodiments, R⁵ is monocyclic 3-, 4-, 5- or 6-membered heterocyclyl having from 1 to 2, 1 to 3, 1 to 4 or 1 to 5 annular carbon atoms and 1 annular heteroatom selected from nitrogen, oxygen and sulfur, which is optionally substituted with 1, 2, 3, 4 or 5 substituents independently selected from R¹⁰. In some embodiments, R⁵ is monocyclic 3- to 6-membered heterocyclyl having 1 annular heteroatom which is oxygen, optionally substituted with 1, 2, 3, 4 or 5 substituents independently selected from R¹⁰.

In some embodiments, R⁵ is C₆₋₁₄ aryl or 5- to 14-membered heteroaryl, each of which is optionally substituted with 1, 2, 3, 4 or 5 substituents independently selected from R¹⁰. In some embodiments, R⁵ is C₆₋₁₄ aryl optionally substituted with 1, 2, 3, 4 or 5 substituents independently selected from R¹⁰. In some embodiments, R⁵ is phenyl optionally substituted with 1, 2, 3, 4 or 5 substituents independently selected from R¹⁰. In some embodiments, R⁵ is 5- to 14-membered heteroaryl having 1 to 12 annular carbon atoms and 1 to 6 annular heteroatoms independently selected from nitrogen, oxygen and sulfur, optionally substituted with 1, 2, 3, 4 or 5 substituents independently selected from R¹⁰. In some embodiments, R⁵ is 5- to 10-membered heteroaryl having 1 to 8 annular carbon atoms and 1 to 4 annular heteroatoms independently selected from nitrogen, oxygen and sulfur, optionally substituted with 1, 2, 3, 4 or 5 substituents independently selected from R¹⁰. In some embodiments, R⁵ is 5- or 6-membered heteroaryl having 1 to 3 annular heteroatoms independently selected from nitrogen, oxygen and sulfur, optionally substituted with 1, 2, 3, 4 or 5 substituents independently selected from R¹⁰. In some embodiments, R⁵ is pyrazolyl (e.g., 3-pyrazolyl, 4-pyrazolyl or 5-pyrazolyl) optionally substituted with 1, 2, 3, 4 or 5 substituents independently selected from R¹⁰.

In some embodiments, R⁵ is a heterocyclyl selected from the group consisting of

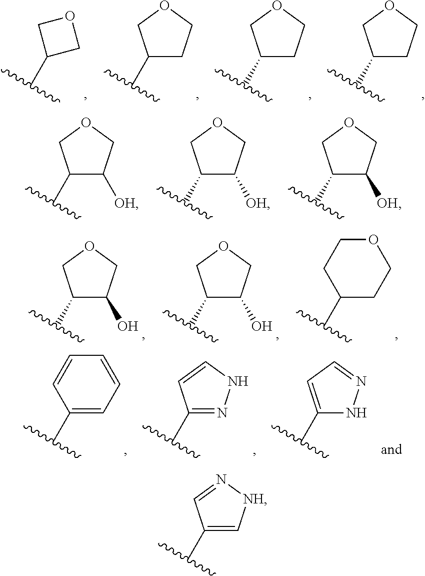

each of which is optionally substituted with 1, 2, 3, 4 or 5 substituents independently selected from R¹⁰, wherein the wavy line in each group indicates the point of attachment to the parent structure.

In some embodiments, R⁵ is hydrogen, C₁₋₆ alkyl, C₃₋₈ cycloalkyl, C₆₋₁₄ aryl, 5- to 14-membered heteroaryl, 3- to 14-membered heterocyclyl or —C(O)R¹⁴, wherein the C₁₋₆ alkyl, C₃₋₈ cycloalkyl, C₆₋₁₄ aryl, 5- to 14-membered heteroaryl and 3- to 14-membered heterocyclyl of R⁵ are each optionally substituted with 1, 2, 3, 4 or 5 substituents independently selected from R¹⁰. In some embodiments, R⁵ is C₁₋₆ alkyl optionally substituted with 1, 2, 3, 4 or 5 substituents independently selected from the group consisting of halogen (e.g., fluoro), cyano, hydroxyl, —O(C₁₋₆ alkyl), —NHC(O)(C₁₋₆ alkyl), —NHS(O)₂(C₁₋₆ alkyl), —S(O)₂NH₂, —C(O)NH₂, phenyl and 3- to 12-membered heterocyclyl (e.g., oxetan-3-yl), C₃₋₆ cycloalkyl substituted with 1, 2, 3, 4 or 5 substituents independently selected from the group consisting of halogen (e.g., fluoro), cyano and hydroxyl, monocyclic 3- to 6-membered heterocyclyl having 1 annular heteroatom which is oxygen, phenyl or pyrazolyl (e.g., 3-pyrazolyl, 4-pyrazolyl or 5-pyrazolyl). In some of these embodiments, R¹⁴ is C₁₋₆ alkyl (e.g., methyl).

In some embodiments, R⁵ is selected from the group consisting of hydrogen, acetyl, methyl, ethyl, 1-propyl, 2-propyl, 2-methyl-1-propyl, 2-methyl-2-propyl,

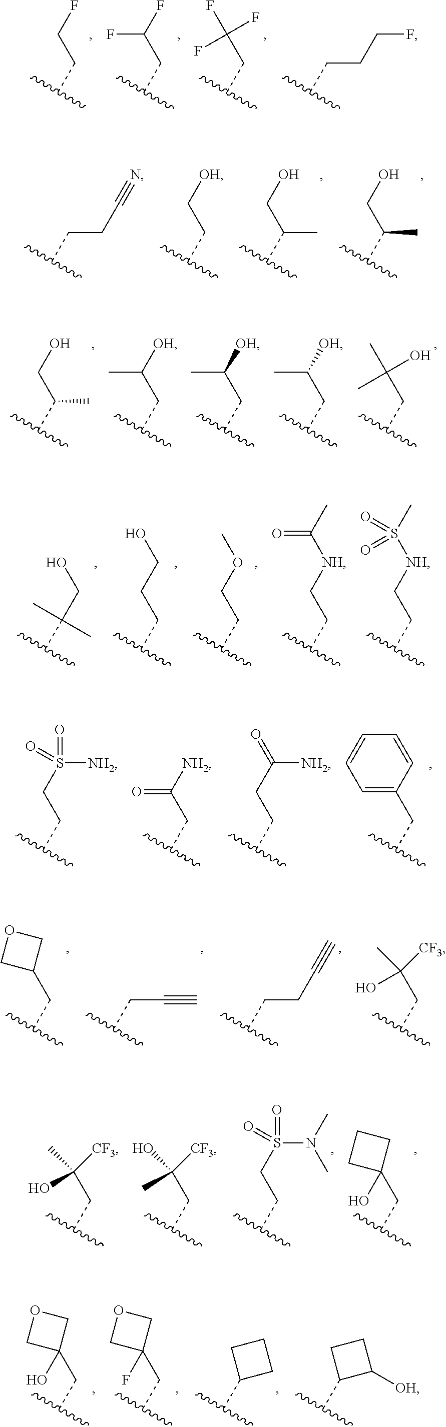

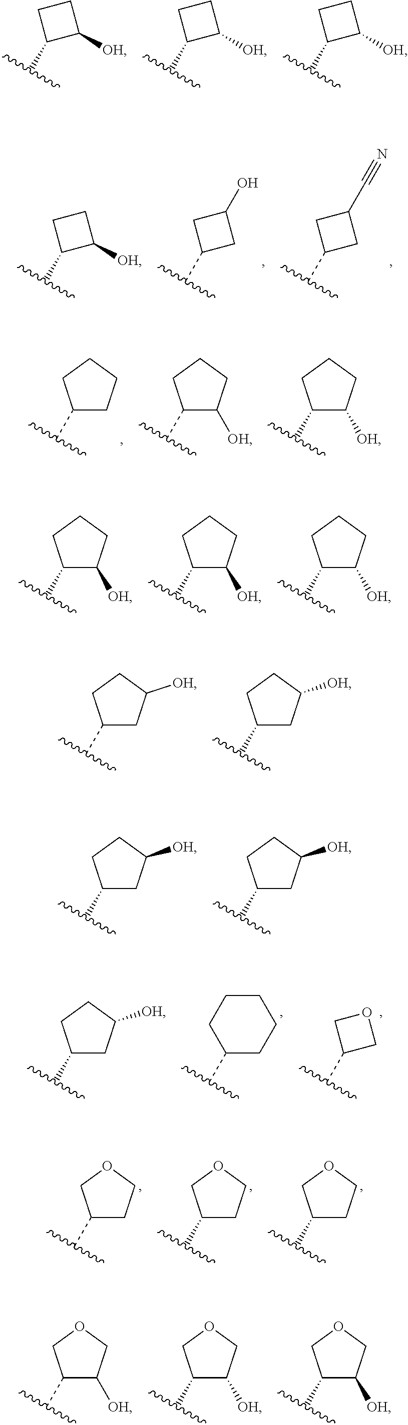

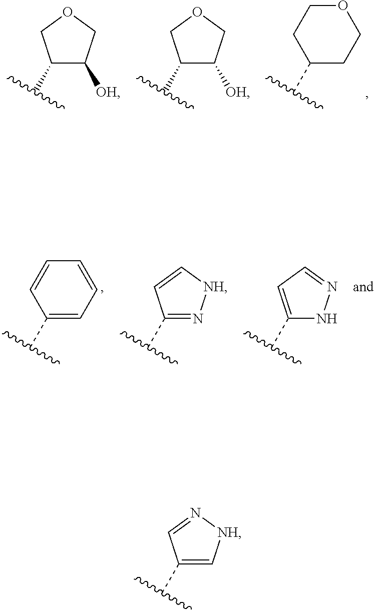

and each of which is optionally substituted with 1, 2, 3, 4 or 5 substituents independently selected from R¹⁰, wherein the wavy line in each group indicates the point of attachment to the parent structure.

In some embodiments, the compound is of the Formula (I), or variations thereof such as Formula (IA), (IB) and (IC), or a salt (e.g., a pharmaceutically acceptable salt) thereof, wherein each R^{6a} and R^{6b} is independently hydrogen, C₁₋₆ alkyl, C₃₋₈ cycloalkyl, C₆₋₁₀ aryl, 5- to 14-membered heteroaryl, 3- to 12-membered heterocyclyl, —C(O)R¹⁴, —C(O)OR¹⁵ or —C(O)NR^{16a}R^{16b}, wherein the C₁₋₆ alkyl, C₃₋₈ cycloalkyl, C₆₋₁₀ aryl, 5- to 14-membered heteroaryl, and 3- to 12-membered heterocyclyl of R^{6a} and R^{6b} are each optionally substituted with 1, 2, 3, 4 or 5 substituents independently selected from R¹⁰; or taken together with R⁵ and the atoms to which they are attached to form a 3- to 14-membered heterocyclyl optionally substituted with 1, 2, 3, 4 or 5 substituents independently selected from R¹⁰; or R^{6a} and R^{6b} are taken together with the carbon to which they are attached to form a carbonyl. In some embodiments, each R^{6a} and R^{6b} is independently hydrogen, C₁₋₆ alkyl, C₃₋₈ cycloalkyl, C₆₋₁₀ aryl, 5- to 14-membered heteroaryl, 3- to 12-membered heterocyclyl, —C(O)R¹⁴, —C(O)OR¹⁵ or —C(O)NR^{16a}R^{16b}, wherein the C₁₋₆ alkyl, C₃₋₈ cycloalkyl, C₆₋₁₀ aryl, 5- to 14-membered heteroaryl, and 3- to 12-membered heterocyclyl of R^{6a} and R^{6b} are each optionally substituted with 1, 2, 3, 4 or 5 substituents independently selected from R¹⁰; or R^{6a} and R^{6b} are taken together with the carbon to which they are attached to form a carbonyl.

In some embodiments, each R^{6a} and R^{6b} is independently hydrogen or C₁₋₆ alkyl. In some embodiments, R^{6a} and R^{6b} are taken together with the carbon to which they are attached to form a carbonyl. In some embodiments, each R^{6a} and R^{6b} is independently hydrogen or C₁₋₆ alkyl; or R^{6a} and R^{6b} are taken together with the carbon to which they are attached to form a carbonyl.

In some embodiments, each R^{6a} and R^{6b} is independently hydrogen, —C(O)OR¹⁵, —C(O)NR^{16a}R^{16b} or C₁₋₆ alkyl optionally substituted with 1, 2, 3, 4 or 5 substituents independently selected from R¹⁰. In some embodiments, each R^{6a} and R^{6b} is independently hydrogen or C₁₋₆ alkyl. In some embodiments, each R^{6a} and R^{6b} is hydrogen. In some embodiments, one of R^{6a} and R^{6b} is hydrogen, and the other one of R^{6a} and R^{6b} is C₁₋₆ alkyl (e.g., methyl). In some embodiments, one of R^{6a} and R^{6b} is hydrogen, and the other one of R^{6a} and R^{6b} is C₁₋₆ alkyl optionally substituted with 1, 2, 3, 4 or 5 substituents independently selected from R¹⁰. In some of these embodiments, R¹⁰ is selected from the group consisting of halogen (e.g., fluoro), —OR^{b} where each R^{b} is independently hydrogen or C₁₋₆ alkyl (e.g., methyl), or —N(R^{g})S(O)₂Rc where Rc is independently C₁₋₆ alkyl (e.g., methyl) and R^{f2} is independently hydrogen or C₁₋₆ alkyl.

In some embodiments, one of R^{6a} and R^{6b} is hydrogen, and the other one of R^{6a} and R^{6b} is hydrogen, —C(O)OR¹⁵, —C(O)NR^{16a}R^{16b} or C₁₋₆ alkyl optionally substituted with 1, 2, 3, 4 or 5 substituents independently selected from R¹⁰. In some embodiments, one of R^{6a} and R^{6b} is hydrogen, and the other one of R^{6a} and R^{6b} is —C(O)OR¹⁵ or —C(O)NR^{16a}R^{16b}. In some embodiments, R¹⁵ is C₁₋₆ alkyl. In some embodiments, one of R^{6a} and R^{6b} is —C(O)O(C₁₋₆ alkyl).

In some embodiments, one of R^{6a} and R^{6b} is hydrogen, and the other one of R^{6a} and R^{6b} is —C(O)NR^{16a}R^{16b}. In some embodiments, each R^{16a} and R^{16b} is independently hydrogen or C₁₋₆ alkyl, or R^{16a} and R^{16b} are taken together with the nitrogen atom to which they are attached to form a 4- to 12-membered heterocyclyl optionally substituted with 1, 2, 3, 4 or 5 substituents independently selected from R¹⁰. In some embodiments, one of R^{6a} and R^{6b} is —C(O)NR^{16a}R^{16b}, where each R^{16a} and R^{16b} is independently hydrogen or C₁₋₆ alkyl (e.g., methyl). In some embodiments, one of R^{6a} and R^{6b} is —C(O)NR^{16a}R^{16b}, where R^{16a} and R^{16b} are taken together with the nitrogen atom to which they are attached to form a 4- to 12-membered heterocyclyl optionally substituted with 1, 2, 3, 4 or 5 substituents independently selected from R¹⁰. In some of these embodiments, R^{16a} and R^{16b} are taken together with the nitrogen atom to which they are attached to form a 4- to 7-membered heterocyclyl having 1 to 3 annular heteroatoms selected from nitrogen, oxygen and sulfur, optionally substituted with 1, 2, 3, 4 or 5 substituents independently selected from R¹⁰. In some of these embodiments, R^{16a} and R^{16b} are taken together with the nitrogen atom to which they are attached to form a 5- or 6-membered heterocyclyl having 1 to 2 annular heteroatoms selected from nitrogen, oxygen and sulfur, optionally substituted with 1, 2, 3, 4 or 5 substituents independently selected from R¹⁰. In some of these embodiments, R^{16a} and R^{16b} are taken together with the nitrogen atom to which they are attached to form pyrrolidin-1-yl or morpholin-4-yl, each of which is optionally substituted with 1, 2, 3, 4 or 5 substituents independently selected from R¹⁰.

In some embodiments, each R^{6a} and R^{6b} is independently hydrogen, C₁₋₆ alkyl (e.g., methyl), —C(O)O(C₁₋₆ alkyl) or —C(O)NR^{16a}R^{6b}, or R^{6a} and R^{6b} are taken together with the carbon to which they are attached to form a carbonyl. In some embodiments, one of R^{6a} and R^{6b} is hydrogen, and the other one of R^{6a} and R^{6b} is hydrogen, C₁₋₆ alkyl (e.g., methyl), —C(O)O(C₁₋₆ alkyl) or —C(O)NR^{16a}R^{6b}, or R^{6a} and R^{6b} are taken together with the carbon to which they are attached to form a carbonyl. In some of these embodiments, each R^{16a} and R^{16b} is independently hydrogen or C₁₋₆ alkyl (e.g., methyl), or R^{16a} and R^{16b} are taken together with the nitrogen atom to which they are attached to form pyrrolidin-1-yl or morpholin-4-yl.

In some embodiments, one of R^{6a} and R^{6b} is selected from the group consisting of hydrogen, methyl,

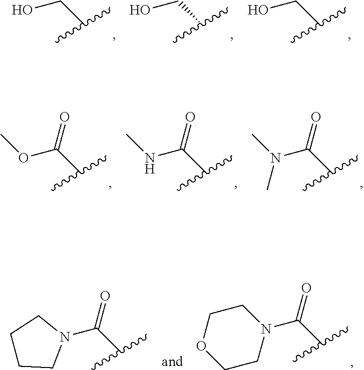

wherein the wavy line in each group indicates the point of attachment to the parent structure. In some embodiments, one of R^{6a} and R^{6b} is selected from the group consisting of

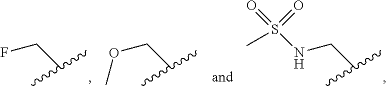

wherein the wavy line in each group indicates the point of attachment to the parent structure.

In some embodiments, one of R^{6a} and R^{6b} is taken together with R⁵ and the atoms to which they are attached to form a 3- to 14-membered heterocyclyl optionally substituted with 1, 2, 3, 4 or 5 substituents independently selected from R¹⁰, and the other one of R^{6a} and R^{6b} is hydrogen, C₁₋₆ alkyl, C₃₋₈ cycloalkyl, C₆₋₁₀ aryl, 5- to 14-membered heteroaryl, 3- to 12-membered heterocyclyl, —C(O)R¹⁴, —C(O)OR¹⁵ or —C(O)NR^{16a}R^{16b}, wherein the C₁₋₆ alkyl, C₃₋₈ cycloalkyl, C₆₋₁₀ aryl, 5- to 14-membered heteroaryl, and 3- to 12-membered heterocyclyl are each optionally substituted with 1, 2, 3, 4 or 5 substituents independently selected from R¹⁰. In some embodiments, one of R^{6a} and R^{6b} is taken together with R⁵ and the atoms to which they are attached to form a 4- to 8-membered heterocyclyl optionally substituted with 1, 2, 3, 4 or 5 substituents independently selected from R¹⁰, and the other one of R^{6a} and R^{6b} is hydrogen or C₁₋₆ alkyl. In some embodiments, one of R^{6a} and R^{6b} is taken together with R⁵ and the atoms to which they are attached to form a 4- to 8-membered heterocyclyl optionally substituted with 1, 2, 3, 4 or 5 substituents independently selected from R¹⁰, and the other one of R^{6a} and R^{6b} is hydrogen. In some embodiments, one of R^{6a} and R^{6b} is taken together with R⁵ and the atoms to which they are attached to form a 5- or 6-membered heterocyclyl (e.g., morpholine) and the other one of R^{6a} and R^{6b} is hydrogen.

In some embodiments, the compound is of the Formula (I), or variations thereof such as Formula (IA), (IB) and (IC), or a salt (e.g., a pharmaceutically acceptable salt) thereof, wherein each R^{7a} and R^{7b} is independently hydrogen or C₁₋₆ alkyl optionally substituted with 1, 2, 3, 4 or 5 substituents independently selected from R¹⁰; or R^{7a} and R^{7b} are taken together with the carbon to which they are attached to form a carbonyl. In some embodiments, each R^{7a} and R^{7b} is independently hydrogen or C₁₋₆ alkyl optionally substituted with 1, 2, 3, 4 or 5 substituents independently selected from R¹⁰.

In some embodiments, each R^{7a} and R^{7b} is independently hydrogen or C₁₋₆ alkyl; or R^{7a} and R^{7b} are taken together with the carbon to which they are attached to form a carbonyl. In some embodiments, R^{7a} and R^{7b} are taken together with the carbon to which they are attached to form a carbonyl.

In some embodiments, each R^{7a} and R^{7b} is independently hydrogen or C₁₋₆ alkyl. In some embodiments, each R^{7a} and R^{7b} is hydrogen. In some embodiments, one of R^{7a} and R^{7b} is hydrogen, and the other one of R^{7a} and R^{7b} is C₁₋₆ alkyl (e.g., methyl).

In some embodiments, one of R^{7a} and R^{7b} is hydrogen, and the other one of R^{7a} and R^{7h} is hydrogen or C₁₋₆ alkyl (e.g., methyl), or R^{7a} and R^{7b} are taken together with the carbon to which they are attached to form a carbonyl.

In some embodiments, the compound is of the Formula (I), or variations thereof such as Formula (IA), (IB) and (IC), or a salt (e.g., a pharmaceutically acceptable salt) thereof, wherein each R^{8a} and R^{8b} is independently hydrogen, halogen, hydroxyl, —O(C₁₋₆ alkyl) or C₁₋₆ alkyl, wherein each C₁₋₆ alkyl is optionally substituted with 1, 2, 3, 4 or 5 substituents independently selected from R¹⁰. In some of these embodiments, one of R^{8a} and R^{8b} is hydrogen, and the other one of R^{8a} and R^{8b} is hydrogen, halogen, hydoxyl, C₁₋₆ alkyl, or —O(C₁₋₆ alkyl). In some of these embodiments, one of R^{8a} and R^{8b} is hydrogen, and the other one of R^{8a} and R^{8b} is hydrogen, halogen (e.g., fluoro) or hydoxyl. In some of these embodiments, one of R^{8a} and R^{8b} is hydrogen and the other one of R^{8a} and R^{8b} is hydrogen, fluoro or hydroxyl. In some embodiments, each R^{8a} and R^{8b} is hydrogen. In some embodiments, each R^{8a} and R^{8b} is fluoro.

It is intended and understood that each and every variation of R¹, R², R³ and R⁴ described for the Formula (I), (IA), (IB) or (IC) may be combined with each and every variation of R⁵, R^{6a}, R^{6b}, R^{7a}, R^{7b}, R^{8a} and R^{8b} described for the Formula (I), (IA), (IB) or (IC), the same as if each and every combination is specifically and individully described. For example, in some embodiments, R¹ is pyrazolyl (e.g., pyrazol-3-yl, pyrazol-4-yl or pyrazol-5-yl), pyridinyl (e.g., 4-pyridyl) or pyrrolo-pyridinyl (e.g., pyrrolo[2,3-b]pyridin-4-yl), each of which is optionally substituted with 1 to 3 substituents independently selected from R¹⁰; R² is hydrogen or C₁₋₆ alkyl (e.g., methyl) optionally substituted with 1 to 5 substituents independently selected from R¹⁰; R³ is hydrogen or C₁₋₆ alkyl (e.g., methyl); R⁴ is hydrogen, halogen or C₁₋₆ alkyl; R⁵ is hydrogen, C₁₋₆ alkyl, C₃₋₈ cycloalkyl, C₆₋₁₄ aryl, 5- to 14-membered heteroaryl, 3- to 14-membered heterocyclyl or —C(O)R¹⁴, wherein the C₁₋₆ alkyl, C₃₋₈ cycloalkyl, C₆₋₁₄ aryl, 5- to 14-membered heteroaryl and 3- to 14-membered heterocyclyl of R⁵ are each optionally substituted with 1, 2, 3, 4 or 5 substituents independently selected from R¹⁰; each R^{6a} and R^{6b} is independently hydrogen, —C(O)OR¹⁵, —C(O)NR^{16a}R^{16b} or C₁₋₆ alkyl optionally substituted with 1, 2, 3, 4 or 5 substituents independently selected from R¹⁰, or R^{6a} and R^{6b} are taken together with the carbon to which they are attached to form a carbonyl; each R^{7a} and R^{7b} is independently hydrogen or C₁₋₆ alkyl, or R^{7a} and R^{7b} are taken together with the carbon to which they are attached to form a carbonyl; one of R^{8a} and R^{8b} is hydrogen, and the other one of R^{8a} and R^{8b} is hydrogen, halogen, hydoxyl, C₁₋₆ alkyl, or —O(C₁₋₆ alkyl). In some of these embodiments, R¹ is pyrazolyl (e.g., pyrazol-3-yl, pyrazol-4-yl or pyrazol-5-yl), isothiazolyl (e.g., isothiazol-5-yl), pyridinyl (e.g., 4-pyridyl) or pyrrolo-pyridinyl (e.g., pyrrolo[2,3-b]pyridin-4-yl), each of which is optionally substituted with 1 to 3 substituents independently selected from R¹⁰; R² is hydrogen or C₁₋₆ alkyl (e.g., methyl) optionally substituted with 1 to 5 substituents independently selected from R¹⁰; R³ is hydrogen, halogen (e.g., chloro) or C₁₋₆ alkyl (e.g., methyl); R⁴ is hydrogen, halogen, —O(C₁₋₆ alkyl) or C₁₋₆ alkyl; R⁵ is hydrogen, C₁₋₆ alkyl, C₃₋₈ cycloalkyl, C₆₋₁₄ aryl, 5- to 14-membered heteroaryl, 3 to 14-membered heterocyclyl or —C(O)R¹⁴, wherein the C₁₋₆ alkyl, C₃₋₈ cycloalkyl, C₆₋₁₄ aryl, 5 to 14-membered heteroaryl and 3- to 14-membered heterocyclyl of R⁵ are each optionally substituted with 1, 2, 3, 4 or 5 substituents independently selected from R¹⁰; each R^{6a} and R^{6b} is independently hydrogen, —C(O)OR¹⁵, —C(O)NR^{16a}R^{16b} or C₁₋₆ alkyl optionally substituted with 1, 2, 3, 4 or 5 substituents independently selected from R¹⁰, or R^{6a} and R^{6b} are taken together with the carbon to which they are attached to form a carbonyl; each R^{7a} and R^{7b} is independently hydrogen or C₁₋₆ alkyl, or R^{7a} and R^{7b} are taken together with the carbon to which they are attached to form a carbonyl; one of R^{8a} and R^{8b} is hydrogen, and the other one of R^{8a} and R^{8b} is hydrogen, halogen, hydoxyl, C₁₋₆ alkyl, or —O(C₁₋₆ alkyl). In some embodiments, R¹⁴ is C₁₋₆ alkyl (e.g., methyl). In some embodiments, R¹⁵ is C₁₋₆ alkyl. In some embodiments, each R^{16a} and R^{16b} is independently hydrogen or C₁₋₆ alkyl (e.g., methyl), or R^{16a} and R^{16b} are taken together with the nitrogen atom to which they are attached to form a 5- or 6-membered heterocyclyl having 1 to 2 annular heteroatoms selected from nitrogen, oxygen and sulfur, optionally substituted with 1, 2, 3, 4 or 5 substituents independently selected from R¹⁰.

In some embodiments, R¹ is pyrazol-4-yl, isothiazol-5-yl, 4-pyridyl or pyrrolo[2,3-b]pyridin-4-yl, each of which is optionally substituted with 1 to 3 substituents independently selected from the group consisting of halogen (e.g., chloro), cyano, unsubstituted C₁₋₆ alkyl (e.g., methyl) and C₁₋₆ haloalkyl (e.g., trifluoromethyl); each R² and R³ is independently hydrogen or C₁₋₆ alkyl (e.g., methyl); R⁴ is hydrogen, halogen (e.g., chloro), —O(C₁₋₆ alkyl) (e.g., methoxy) or C₁₋₆ alkyl (e.g., methyl); R⁵ is (i) C₁₋₆ alkyl optionally substituted with 1, 2, 3, 4 or 5 substituents independently selected from the group consisting of halogen (e.g., fluoro), cyano, hydroxyl, —O(C₁₋₆ alkyl), —NHC(O)(C₁₋₆ alkyl), —NHS(O)₂(C₁₋₆ alkyl), —S(O)₂NH₂, —C(O)NH₂, phenyl and 3- to 12-membered heterocyclyl (e.g., oxetan-3-yl), (ii) C₃₋₆ cycloalkyl substituted with 1, 2, 3, 4 or 5 substituents independently selected from the group consisting of halogen (e.g., fluoro), cyano and hydroxyl, (iii) monocyclic 3- to 6-membered heterocyclyl having 1 annular heteroatom which is oxygen, (iv) phenyl, or (v) pyrazolyl (e.g., 3-pyrazolyl, 4-pyrazolyl or 5-pyrazolyl); one of R^{6a} and R^{6b} is hydrogen, and the other one of R^{6a} and R^{6b} is hydrogen, C₁₋₆ alkyl (e.g., methyl), —C(O)O(C₁₋₆ alkyl) or —C(O)NR^{16a}R^{16b}, or R^{6a} and R^{6b} are taken together with the carbon to which they are attached to form a carbonyl; one of R^{7a} and R^{7b} is hydrogen, and the other one of R^{7a} and R^{7b} is hydrogen or C₁₋₆ alkyl (e.g., methyl), or R^{7a} and R^{7b} are taken together with the carbon to which they are attached to form a carbonyl; and R^{8a} and R^{8b} are hydrogen. In some embodiments, R¹ is pyrazol-4-yl, 4-pyridyl or pyrrolo[2,3-b]pyridin-4-yl, each of which is optionally substituted with 1 to 3 substituents independently selected from the group consisting of halogen (e.g., chloro), cyano, unsubstituted C₁₋₆ alkyl (e.g., methyl) and C₁₋₆ haloalkyl (e.g., trifluoromethyl); each R² and R³ is independently hydrogen or C₁₋₆ alkyl (e.g., methyl); R⁴ is hydrogen, halogen (e.g., chloro) or C₁₋₆ alkyl (e.g., methyl); R⁵ is (i) C₁₋₆ alkyl optionally substituted with 1, 2, 3, 4 or 5 substituents independently selected from the group consisting of halogen (e.g., fluoro), cyano, hydroxyl, —O(C₁₋₆ alkyl), —NHC(O)(C₁₋₆ alkyl), —NHS(O)₂(C₁₋₆ alkyl), —S(O)₂NH₂, —C(O)NH₂, phenyl and 3- to 12-membered heterocyclyl (e.g., oxetan-3-yl), (ii) C₃₋₆ cycloalkyl substituted with 1, 2, 3, 4 or 5 substituents independently selected from the group consisting of halogen (e.g., fluoro), cyano and hydroxyl, (iii) monocyclic 3- to 6-membered heterocyclyl having 1 annular heteroatom which is oxygen, (iv) phenyl, or (v) pyrazolyl (e.g., 3-pyrazolyl, 4-pyrazolyl or 5-pyrazolyl); one of R^{6a} and R^{6b} is hydrogen, and the other one of R^{6a} and R^{6b} is hydrogen, C₁₋₆ alkyl (e.g., methyl), —C(O)O(C₁₋₆ alkyl) or —C(O)NR^{16a}R^{16b}, or R^{6a} and R^{6b} are taken together with the carbon to which they are attached to form a carbonyl; one of R^{7a} and R^{7b} is hydrogen, and the other one of R^{7a} and R^{7I} is hydrogen or C₁₋₆ alkyl (e.g., methyl), or R^{7a} and R^{7I} are taken together with the carbon to which they are attached to form a carbonyl; and R^{8a} and R^{8b} are hydrogen. In some embodiments, each R^{16a} and R^{16b} is independently hydrogen or C₁₋₆ alkyl (e.g., methyl), or R^{16a} and R^{16b} are taken together with the nitrogen atom to which they are attached to form pyrrolidin-1-yl or morpholin-4-yl.

In some embodiments, the compound is of the Formula (I), or variations thereof such as Formula (IA), (IB) and (IC), or a salt (e.g., a pharmaceutically acceptable salt) thereof, wherein n is 0 to 8. The piperidine moiety of the spirocycle is unsubstituted (n is 0) or is substituted with 1 to 8 R^{g} groups (n=1, 2, 3, 4, 5, 6, 7 or 8). In some embodiments, each R^{g}, when present, is independently C₁₋₆ alkyl, or two geminal R^{g} groups, if present, are taken together with the carbon to which they are attached to form a carbonyl.

It is intended and understood that each and every variation of R¹, R², R³, R⁴, R⁵, R^{6a}, R^{6b}, R^{7a}, R^{7b}, R^{8a} and R^{8b} described for the Formula (I), (IA), (IB) or (IC) may be combined with R^{g} and n described for the Formula (I), (IA), (IB) or (IC), the same as if each and every combination is specifically and individually described. For example, in some embodiments, R¹, R², R³, R⁴, R⁵, R^{6a}, R^{6b}, R^{7a}, R^{7b}, R^{8a} and R^{8b} are as detailed herein, or any combinations thereof detailed herein, and n is 0 (i.e., R^{g} is absent).

In some embodiments, the compound of the Formula (I) is of the Formula (II):

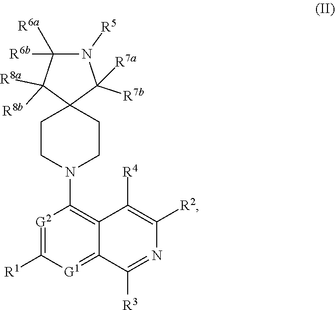

or a pharmaceutically acceptable salt thereof, wherein R¹, R², R³, R⁴, G¹, G², R⁵, R^{6a}, R^{6b}, R^{7a}, R^{7b}, R^{8a} and R^{8b} are as detailed herein for Formula (I), or variations detailed herein.

In some embodiments, the compound of the Formula (I) or (II) is of the Formula (II-A):

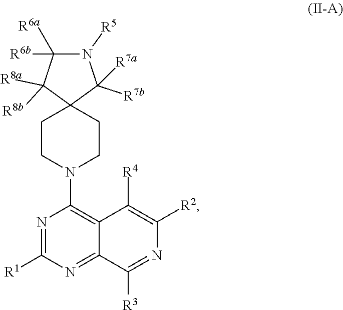

or a pharmaceutically acceptable salt thereof, wherein R¹, R², R³, R⁴, R⁵, R^{6a}, R^{6b}, R^{7a}, R^{7b}, R^{8a}, and R^{8b} are as detailed herein for Formula (I) or (II), or variations detailed herein.

In some embodiments, the compound of the Formula (I) or (II) is of the Formula (II-B):

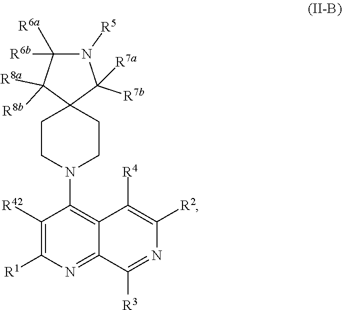

or a pharmaceutically acceptable salt thereof, wherein R¹, R², R³, R⁴, R⁴², R⁵, R^{6a}, R^{6b}, R^{7a}, R^{7b}, R^{8a} and R^{8b} are as detailed herein for Formula (I) or (II), or variations detailed herein.

In some embodiments, the compound of the Formula (I) or (II) is of the Formula (II-C).

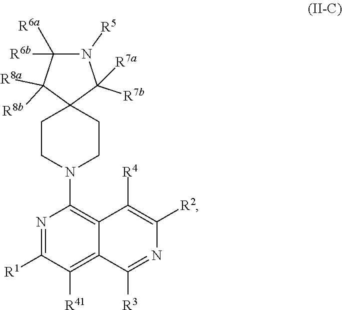

or a pharmaceutically acceptable salt thereof, wherein R¹, R², R³, R⁴, R⁴¹, R⁵, R^{6a}, R^{6b}, R^{7a}, R^{7b}, R^{8a} and R^{8b} are as detailed herein for Formula (I) or (II), or variations detailed herein.

In some embodiments, the compound is of the Formula (II), (II-A), (II-B) or (II-C), or a salt (e.g., a pharmaceutically acceptable salt) thereof, wherein R¹ is pyrazolyl (e.g., pyrazol-3-yl, pyrazol-4-yl or pyrazol-5-yl), pyridinyl (e.g., 4-pyridyl) or pyrrolo-pyridinyl (e.g., pyrrolo[2,3-b]pyridin-4-yl), each of which is optionally substituted with 1 to 3 substituents independently selected from R¹⁰; R² is hydrogen or C₁₋₆ alkyl (e.g., methyl) optionally substituted with 1 to 5 substituents independently selected from R¹⁰; R³ is hydrogen or C₁₋₆ alkyl (e.g., methyl); R⁴ is hydrogen, halogen or C₁₋₆ alkyl; R⁵ is hydrogen, C₁₋₆ alkyl, C₃₋₈ cycloalkyl, C₆₋₁₄ aryl, 5- to 14-membered heteroaryl, 3- to 14-membered heterocyclyl or —C(O)R¹⁴, wherein the C₁₋₆ alkyl, C₃₋₈ cycloalkyl, C₆₋₁₄ aryl, 5- to 14-membered heteroaryl and 3- to 14-membered heterocyclyl of R⁵ are each optionally substituted with 1, 2, 3, 4 or 5 substituents independently selected from R¹⁰; each R^{6a} and R^{6b} is independently hydrogen, —C(O)OR¹⁵, —C(O)NR^{16a}R^{16b} or C₁₋₆ alkyl optionally substituted with 1, 2, 3, 4 or 5 substituents independently selected from R¹⁰, or R^{6a} and R^{6b} are taken together with the carbon to which they are attached to form a carbonyl; each R^{7a} and R^{7b} is independently hydrogen or C₁₋₆ alkyl, or R^{7a} and R^{7b} are taken together with the carbon to which they are attached to form a carbonyl; one of R^{8a} and R^{8b} is hydrogen, and the other one of R^{8a} and R^{8b} is hydrogen, halogen, hydoxyl, C₁₋₆ alkyl, or —O(C₁₋₆ alkyl). In some embodiments, R¹⁴ is C₁₋₆ alkyl (e.g., methyl). In some embodiments, R¹⁵ is C₁₋₆ alkyl. In some embodiments, each R^{16a} and R^{16b} is independently hydrogen or C₁₋₆ alkyl (e.g., methyl), or R^{16a} and R^{16b} are taken together with the nitrogen atom to which they are attached to form a 5- or 6-membered heterocyclyl having 1 to 2 annular heteroatoms selected from nitrogen, oxygen and sulfur, optionally substituted with 1, 2, 3, 4 or 5 substituents independently selected from R¹⁰.

In some embodiments, the compound is of the Formula (II), (II-A), (II-B) or (II-C), or a salt (e.g., a pharmaceutically acceptable salt) thereof, wherein R¹ is pyrazol-4-yl, isothiazol-5-yl, 4-pyridyl or pyrrolo[2,3-b]pyridin-4-yl, each of which is optionally substituted with 1 to 3 substituents independently selected from the group consisting of halogen (e.g., chloro), cyano, unsubstituted C₁₋₆ alkyl (e.g., methyl) and C₁₋₆ haloalkyl (e.g., trifluoromethyl); R² is independently hydrogen or C₁₋₆ alkyl (e.g., methyl); R³ is independently hydrogen, halogen (e.g., chloro) or C₁₋₆ alkyl (e.g., methyl); R⁴ is hydrogen, halogen (e.g., chloro), —O(C₁₋₆ alkyl) (e.g., methoxyl) or C₁₋₆ alkyl (e.g., methyl); R⁵ is (i) C₁₋₆ alkyl optionally substituted with 1, 2, 3, 4 or 5 substituents independently selected from the group consisting of halogen (e.g., fluoro), cyano, hydroxyl, —O(C₁₋₆ alkyl), —NHC(O)(C₁₋₆ alkyl), —NHS(O)₂(C₁₋₆ alkyl), —S(O)₂NH₂, —C(O)NH₂, phenyl and 3- to 12-membered heterocyclyl (e.g., oxetan-3-yl), (ii) C₃₋₆ cycloalkyl substituted with 1, 2, 3, 4 or 5 substituents independently selected from the group consisting of halogen (e.g., fluoro), cyano and hydroxyl, (iii) monocyclic 3- to 6-membered heterocyclyl having 1 annular heteroatom which is oxygen, (iv) phenyl, or (v) pyrazolyl (e.g., 3-pyrazolyl, 4-pyrazolyl or 5-pyrazolyl); one of R^{6a} and R^{6b} is hydrogen, and the other one of R^{6a} and R^{6b} is hydrogen, C₁₋₆ alkyl (e.g., methyl), —C(O)O(C₁₋₆ alkyl) or —C(O)NR^{16a}R^{6b}, or R^{6a} and R b are taken together with the carbon to which they are attached to form a carbonyl; one of R^{7a} and R^{7b} is hydrogen, and the other one of R^{7a} and R^{7b} is hydrogen or C₁₋₆ alkyl (e.g., methyl), or R^{7a} and R^{7b} are taken together with the carbon to which they are attached to form a carbonyl; and R^{8a} and R^{8b} are hydrogen. In some embodiments, R¹ is pyrazol-4-yl, 4-pyridyl or pyrrolo[2,3-b]pyridin-4-yl, each of which is optionally substituted with 1 to 3 substituents independently selected from the group consisting of halogen (e.g., chloro), cyano, unsubstituted C₁₋₆ alkyl (e.g., methyl) and C₁₋₆ haloalkyl (e.g., trifluoromethyl); each R² and R³ is independently hydrogen or C₁₋₆ alkyl (e.g., methyl); R⁴ is hydrogen, halogen (e.g., chloro) or C₁₋₆ alkyl (e.g., methyl); R⁵ is (i) C₁₋₆ alkyl optionally substituted with 1, 2, 3, 4 or 5 substituents independently selected from the group consisting of halogen (e.g., fluoro), cyano, hydroxyl, —O(C₁₋₆ alkyl), —NHC(O)(C₁₋₆ alkyl), —NHS(O)₂(C₁₋₆ alkyl), —S(O)₂NH₂, —C(O)NH₂, phenyl and 3- to 12-membered heterocyclyl (e.g., oxetan-3-yl), (ii) C₃₋₆ cycloalkyl substituted with 1, 2, 3, 4 or 5 substituents independently selected from the group consisting of halogen (e.g., fluoro), cyano and hydroxyl, (iii) monocyclic 3- to 6-membered heterocyclyl having 1 annular heteroatom which is oxygen, (iv) phenyl, or (v) pyrazolyl (e.g., 3-pyrazolyl, 4-pyrazolyl or 5-pyrazolyl); one of R^{6a} and R^{6b} is hydrogen, and the other one of R^{6a} and R^{6b} is hydrogen, C₁₋₆ alkyl (e.g., methyl), —C(O)O(C₁₋₆ alkyl) or —C(O)NR^{16a}R^{6b}, or R^{6a} and R b are taken together with the carbon to which they are attached to form a carbonyl; one of R^{7a} and R^{7b} is hydrogen, and the other one of R^{7a} and R^{7b} is hydrogen or C₁₋₆ alkyl (e.g., methyl), or R^{7a} and R^{7b} are taken together with the carbon to which they are attached to form a carbonyl; and R^{8a} and R^{8b} are hydrogen. In some embodiments, each R^{16a} and R^{16b} is independently hydrogen or C₁₋₆ alkyl (e.g., methyl), or R^{16a} and R^{16b} are taken together with the nitrogen atom to which they are attached to form pyrrolidin-1-yl or morpholin-4-yl.

In some embodiments, the compound of the Formula (I), (IA), (II) or (II-A) is of the Formula (III):

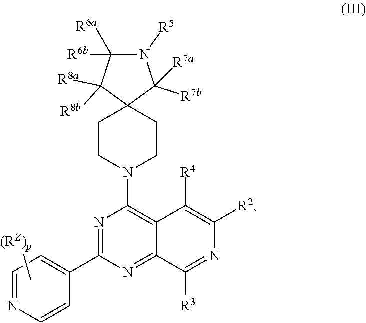

or a pharmaceutically acceptable salt thereof, wherein R², R³, R⁴, R⁵, R^{6a}, R^{6b}, R^{7a}, R^{7b}, R^{8a} and R^{8b} are as detailed herein for Formula (I), (IA), (II) or (II-A), or applicable variations thereof, p is 0, 1, 2, 3 or 4; and each R^{Z} is independently hydrogen, halogen, cyano or C₁₋₆ alkyl optionally substituted with 1, 2, 3, 4 or 5 substituents independently selected from R¹⁰. In some embodiments, p is 0 (R^{Z} is absent). In some embodiments, p is 1 and R^{Z} is fluoro (e.g., 3-fluoro) or cyano (e.g., 3-cyano). In some of these embodiments, each R², R³ and R⁴ is hydrogen.

In some embodiments, the compound of the Formula (I), (IA), (II) or (II-A) is of the Formula (IV):

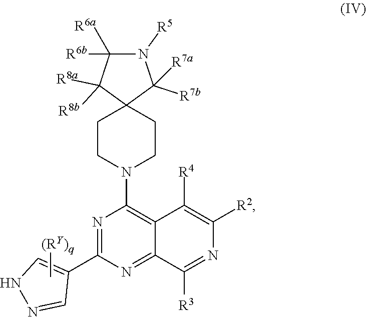

or a pharmaceutically acceptable salt thereof, wherein R², R³, R⁴, R⁵, R^{6a}, R^{6b}, R^{7a}, R^{7b}, R^{8a} and R^{8b} are as detailed herein for Formula (I), (IA), (II) or (II-A), or applicable variations thereof, q is 0, 1, 2 or 3; and each R^{Y} is independently hydrogen, halogen, cyano, —O(C₁₋₆ alkyl) or C₁₋₆ alkyl, wherein the C₁₋₆ alkyl of R is optionally substituted with 1, 2, 3, 4 or 5 substituents independently selected from R¹⁰. In some embodiments, each R^{Y} is independently hydrogen, halogen, cyano or C₁₋₆ alkyl optionally substituted with 1, 2, 3, 4 or 5 substituents independently selected from R¹⁰. In some embodiments, q is 1. In some embodiments, q is 1 and R^{Y} is methyl, fluoro, chloro, cyano or trifluoromethyl. In some embodiments, R^{Y} is attached to the pyrazol-4-yl at the 3- or 5-position. In one variation, R^{Y} is 5-methyl or 3-methyl. In some of these embodiments, each R², R³ and R⁴ is hydrogen.

In some embodiments, the compound of the Formula (I), (IA), (II), (II-A) or (III) is of the Formula (V):

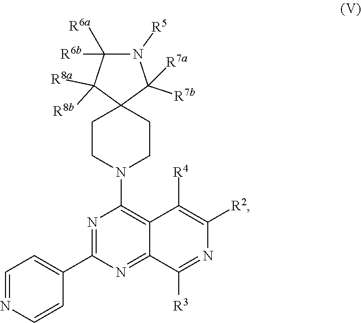

or a pharmaceutically acceptable salt thereof, wherein R², R³, R⁴, R⁵, R^{6a}, R^{6b}, R^{7a}, R^{7b}, R^{8a} and R^{8b} are as detailed herein for Formula (I), (IA), (II), (II-A) or (III), or applicable variations thereof. In some of these embodiments, each R², R³ and R⁴ is hydrogen, and R⁵, R^{6a}, R^{6b}, R^{7a}, R^{7b}, R^{8a} and R^{8b} are as detailed herein for Formula (I), (IA), (II), (II-A) or (III), or applicable variations thereof.

In some embodiments, the compound of the Formula (I), (IA), (II), (II-A) or (IV) is of the Formula (VI):

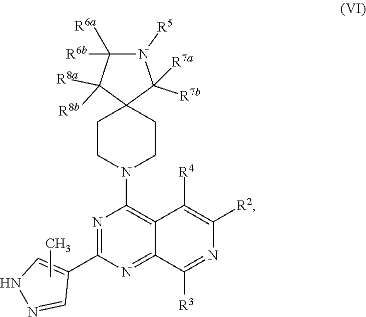

or a pharmaceutically acceptable salt thereof, wherein R², R³, R⁴, R⁵, R^{6a}, R^{6b}, R^{7a}, R^{7b}, R^{8a} and R^{8b} are as detailed herein for Formula (I), (IA), (II), (II-A) or (IV), or applicable variations thereof. In some of these embodiments, each R², R³ and R⁴ is hydrogen, and R⁵, R^{6a}, R^{6b}, R^{7a}, R^{7b}, R^{8a} and R^{8b} are as detailed herein for Formula (I), (IA), (II), (II-A) or (IV), or applicable variations thereof.

In some embodiments, the compound of the Formula (I), (IB), (II) or (II-B) is of the Formula (VII) or (VIII):

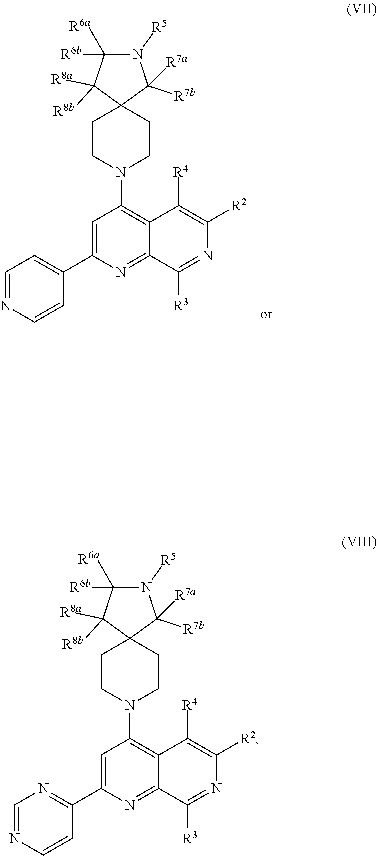

or a pharmaceutically acceptable salt thereof, wherein R², R³, R⁴, R⁵, R^{6a}, R^{6b}, R^{7a}, R^{7b}, R^{8a} and R^{8b} are as detailed herein for Formula (I), (IB), (II) or (II-B), or applicable variations thereof. In some of these embodiments, each R², R³ and R⁴ is hydrogen, and R⁵, R^{6a}, R^{6b}, R^{7a}, R^{7b}, R^{8a} and R^{8b} are as detailed herein for Formula (I), (IB), (II) or (II-B), or applicable variations thereof.

In some embodiments, the compound of the Formula (I), (IC), (II) or (II-C) is of the Formula (IX):

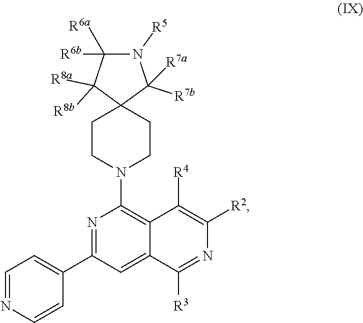

or a pharmaceutically acceptable salt thereof, wherein R², R³, R⁴, R⁵, R^{6a}, R^{6b}, R^{7a}, R^{7b}, R^{8a} and R^{8b} are as detailed herein for Formula (I), (IC), (II) or (II-C), or applicable variations thereof. In some of these embodiments, each R², R³ and R⁴ is hydrogen, and R⁵, R^{6a}, R^{6b}, R^{7a}, R^{7b}, R^{8a} and R^{8b} are as detailed herein for Formula (I), (IC), (II) or (II-C), or applicable variations thereof. In some of these embodiments, each R^{7a} and R^{7b} is independently hydrogen or C₁₋₆ alkyl optionally substituted with 1, 2, 3, 4 or 5 substituents independently selected from R¹⁰.

In some embodiments of the compound of the Formula (I), or variations thereof such as Formula (II), or a salt (e.g., a pharmaceutically acceptable salt) thereof, each R¹⁰ is independently oxo, C₁₋₆ alkyl, C₂₋₆ alkenyl, C₂₋₆ alkynyl, C₃₋₈ cycloalkyl, C₆₋₁₄ aryl, 5- to 14-membered heteroaryl, 3- to 14-membered heterocyclyl, halogen, cyano, —C(O)R^{a}, —C(O)OR^{b}, —C(O)NR^{c}R^{d}, —OR^{b}, —OC(O)R^{a}, —OC(O)NR^{c}R^{d}, —SR^{b}, —S(O)R^{c}, —S(O)₂R^{c}, —S(O)(═NH)R^{c}, —S(O)₂NR^{c}R^{d}, —NRR^{d}, —N(R^{f})C(O)R^{a}, —N(R^{f})C(O)OR^{b}, —N(R^{f})C(O)NR^{c}R^{d}, —N(R^{f})S(O)₂R^{c}, or —N(R^{f})S(O)₂NR^{c}R^{d}; wherein the C₁₋₆ alkyl, C₂₋₆ alkenyl, C₂₋₆ alkynyl, C₃₋₈ cycloalkyl, C₆₋₁₄ aryl, 5- to 14-membered heteroaryl and 3- to 14-membered heterocyclyl of R¹⁰ are each optionally substituted with 1, 2, 3 or 4 substituents independently selected from R^{D}.

In one variation, R¹⁰ is independently oxo, C₁₋₆ alkyl, C₃₋₈ cycloalkyl, C₆₋₁₀ aryl, 5- to 10-membered heteroaryl, 3- to 12-membered heterocyclyl, halogen, cyano, —C(O)R^{a}, —C(O)OR^{b}, —C(O)NRR^{d}, —OR^{b}, —OC(O)R^{a}, —OC(O)NR^{c}R^{d}, —S(O)₂R^{c}, —S(O)₂NR^{c}R^{d}, —NRR^{d}, —N(R^{f})C(O)R^{a}, —N(R^{f})C(O)OR^{b}, —N(R^{f})C(O)NRR^{d}, —N(R^{f})S(O)₂R^{c}, or —N(R^{f})S(O)₂NR^{c}R^{d}; wherein the C₁₋₆ alkyl, C₃₋₈ cycloalkyl, C₆₋₁₀ aryl, 5- to 10-membered heteroaryl and 3- to 12-membered heterocyclyl of R¹⁰ are each optionally substituted with 1, 2, 3 or 4 substituents independently selected from R^{D}.

In one variation, R¹⁰ is independently halogen (e.g., chloro or fluoro), cyano, —OR^{b}, —N(R^{f})C(O)R^{a}, —N(R^{f})S(O)₂R^{c}, —S(O)₂NR^{c}R^{d}, —C(O)NRR^{d}, C₁₋₆ alkyl optionally substituted with 1, 2, 3 or 4 substituents independently selected from R¹¹, C₃₋₈ cycloalkyl optionally substituted with 1, 2, 3 or 4 substituents independently selected from R¹¹, C₆₋₁₀ aryl optionally substituted with 1, 2, 3 or 4 substituents independently selected from R¹¹, 5- to 10-membered heteroaryl optionally substituted with 1, 2, 3 or 4 substituents independently selected from R¹¹, or 3- to 12-membered heterocyclyl optionally substituted with 1, 2, 3 or 4 substituents independently selected from R^{D}.

In one variation, R¹⁰ is independently halogen (e.g., chloro or fluoro), cyano, or hydroxyl.

In one variation, R¹⁰ is independently halogen (e.g., fluoro or chloro), cyano and C₁₋₆ alkyl optionally substituted with halogen (e.g., methyl or trifluoromethyl).

In one variation, R¹⁰ is hydroxyl, cyano, fluoro, chloro, —CH₂F, —CHF₂, —CF₃, —NH₂, —NH(C₁₋₆ alkyl), —N(C₁₋₆ alkyl)₂, —O(C₁₋₆ alkyl), —SO₂(C₁₋₆ alkyl), —S(O)₂NR^{c}R^{d}, —C(O)NR^{c}R^{d}, or —N(R^{f})C(O)R^{a}.

In one variation, R¹⁰ is C₂₋₆ alkenyl (e.g., ethenyl) or C₂₋₆ alkynyl (e.g., ethynyl), each is optionally substituted with 1, 2, 3 or 4 substituents independently selected from R¹.

In one variation, R¹⁰ is independently halogen (e.g., fluoro or choloro), cyano, —OR^{b}, —N(R^{f})C(O)R^{a}, —N(R^{f})S(O)₂R^{c}, —S(O)₂NR^{c}R^{d}, —C(O)NR^{c}R^{d}, C₆₋₁₀ aryl optionally substituted with 1, 2, 3 or 4 substituents independently selected from R¹¹ or 3- to 12-membered heterocyclyl optionally substituted with 1, 2, 3 or 4 substituents independently selected from R¹¹. In some of these embodiments, R^{a} is C₁₋₆ alkyl, R^{b} is hydrogen or C₁₋₆ alkyl, R^{c} is C₁₋₆ alkyl, R^{f2} is hydrogen, each R^{c} and R^{d} is independently hydrogen or C₁₋₆ alkyl, or R^{c} and R^{d} are taken together with the nitrogen atom to which they are attached to form a 4- to 7-membered heterocyclyl having 1 to 3 annular heteroatoms selected from nitrogen, oxygen and sulfur, optionally substituted with 1, 2, 3 or 4 substituents independently selected from R^{D}.

In some embodiments, where a group (e.g., R¹) comprises a 5- to 14-membered heteroaryl optionally substituted with 1 to 5 (e.g., 1, 2, 3, 4 or 5; 1, 2, 3 or 4; or 1, 2 or 3; or 1 or 2) substituents independently selected from R¹⁰, R¹⁰ is selected from the group consisting of halogen (e.g., fluoro or chloro), cyano, oxo, C₁₋₆ alkyl, C₁₋₆ haloalkyl and —OR^{b} where R^{b} is hydrogen or C₁₋₆ alkyl.

In some embodiments, where a group (e.g., R², R³, R⁴, R⁴⁴, R⁵, R^{6a}, R^{6b}, R^{7a}, R^{7b}, R^{8a}, R^{8b} or R^{g}) comprises a C₁₋₆ alkyl optionally substituted with 1 to 5 (e.g., 1, 2, 3, 4 or 5; 1, 2, 3 or 4; or 1, 2 or 3; or 1 or 2) substituents independently selected from R¹⁰, each R¹⁰ is independently C₂₋₆ alkenyl, C₂₋₆ alkynyl, C₃₋₈ cycloalkyl, C₆₋₁₄ aryl, 5- to 14-membered heteroaryl, 3- to 14-membered heterocyclyl, halogen, cyano, —C(O)OR^{b}, —C(O)NR^{c}R^{d}, —OR^{b}, —S(O)₂NR^{c}R^{d}, —NR^{c}R^{d}, —N(R^{f})C(O)R^{a} or —N(R^{f})S(O)₂R^{c}, wherein the C₂₋₆ alkenyl, C₂₋₆ alkynyl, C₃₋₈ cycloalkyl, C₆₋₁₄ aryl, 5- to 14-membered heteroaryl and 3- to 14-membered heterocyclyl of R¹⁰ are each optionally substituted with 1, 2, 3 or 4 substituents independently selected from R¹¹. In some of these embodiments, R¹¹ is selected from the group consisting of halogen (e.g., fluoro or chloro), cyano, C₁₋₆ alkyl, C₁₋₆ haloalkyl and —OR^{b} where R^{b} is hydrogen or C₁₋₆ alkyl. In some of these embodiments, R¹¹ is selected from the group consisting of halogen (e.g., fluoro or chloro), cyano and hydroxyl. In one variation (e.g., with regard to a substituted C₁₋₆ alkyl in R²), R¹⁰ is selected from the group consisting of halogen (e.g., fluoro or chloro), C₆₋₁₄ aryl (e.g., phenyl) optionally substituted with halogen or C₁₋₆ alkyl, 5- to 14-membered heteroaryl (e.g., pyridyl or pyrozolyl) optionally substituted with halogen or C₁₋₆ alkyl, —OR^{b} where R^{b} is hydrogen or C₁₋₆ alkyl and —N(R^{f})C(O)R^{a} where R^{a} is C₁₋₆ alkyl and R^{f2} is hydrogen. In one variation (e.g., with regard to a substituted C₁₋₆ alkyl in R³), R¹⁰ is selected from the group consisting of halogen (e.g., fluoro or chloro) and —OR^{b} where R^{b} is hydrogen or C₁₋₆ alkyl. In one variation (e.g., with regard to a substituted C₁₋₆ alkyl in R⁴ or R⁴⁴), R¹⁰ is selected from the group consisting of halogen (e.g., fluoro or chloro), C₂₋₆ alkenyl, C₂₋₆ alkynyl, —OR^{b} where R^{b} is hydrogen or C₁₋₆ alkyl and —C(O)NR^{c}R^{d} where R^{c} and R^{d} are independently hydrogen or C₁₋₆ alkyl. In one variation (e.g., with regard to a substituted C₁₋₆ alkyl in R⁵), R¹⁰ is selected from the group consisting of halogen (e.g., fluoro or chloro), C₂₋₆ alkenyl (e.g., ethenyl), C₂₋₆ alkynyl (e.g., ethynyl), C₃₋₅ cycloalkyl optionally substituted with halogen, cyano or hydroxyl, C₆₋₁₄ aryl (e.g., phenyl) optionally substituted with halogen, 4- or 5-membered heterocyclyl(e.g., oxetanyl or azetidinyl) optionally substituted with halogen, hydroxyl or acetyl, —C(O)NR^{c}R^{d} where R^{c} and R^{d} are independently hydrogen or C₁₋₆ alkyl, —OR^{b} where R^{b} is hydrogen or C₁₋₆ alkyl, —S(O)₂NR^{c}R^{d} where R^{c} and R^{d} are independently hydrogen or C₁₋₆ alkyl and —N(R^{f})C(O)R^{a} where R^{a} is C₁₋₆ alkyl and R¹ is hydrogen. In one variation (e.g., with regard to a substituted C₁₋₆ alkyl in R^{6a}, R^{6b}, R^{7a}, R^{7b}, R^{8a}, R^{8b} or R^{g}), R¹⁰ is selected from the group consisting of halogen (e.g., fluoro or chloro) and —OR^{b} where R^{b} is hydrogen or C₁₋₆ alkyl.

In some embodiments, where a group (e.g., R⁵) comprises a C₃₋₈ cycloalkyl optionally substituted with 1 to 5 (e.g., 1, 2, 3, 4 or 5; 1, 2, 3 or 4; or 1, 2 or 3; or 1 or 2) substituents independently selected from R¹⁰, R¹⁰ is selected from the group consisting of halogen (e.g., fluoro or chloro), cyano and —OR^{b} where R^{b} is hydrogen or C₁₋₆ alkyl.

In some embodiments, each R^{a} is independently hydrogen, C₁₋₆ alkyl, C₂₋₆ alkenyl, C₂₋₆ alkynyl, C₃₋₈ cycloalkyl, C₆₋₁₀ aryl, 5- to 10-membered heteroaryl or 3- to 12-membered heterocyclyl; wherein the C₁₋₆ alkyl, C₂₋₆ alkenyl, C₂₋₆ alkynyl, C₃₋₈ cycloalkyl, C₆₋₁₀ aryl, 5- to 10-membered heteroaryl and 3- to 12-membered heterocyclyl of R^{a} are each optionally substituted with 1, 2, 3 or 4 substituents independently selected from R¹¹. In one variation, R^{a} is independently hydrogen or C₁₋₆ alkyl.

In some embodiments, each R^{b} is independently hydrogen, C₁₋₆ alkyl, C₃— cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{b} are each optionally substituted with 1, 2, 3 or 4 substituents independently selected from R¹¹. In one variation, R^{b} is independently hydrogen or C₁₋₆ alkyl.

In some embodiments, each R^{c} and R^{d} is independently hydrogen, C₁₋₆ alkyl, C₃₋₈ cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{c} and R^{d} are each optionally substituted with 1, 2, 3 or 4 substituents independently selected from R¹¹; or R^{c} and R^{d} are taken together with the nitrogen atom to which they are attached to form a 4- to 12-membered heterocyclyl optionally substituted with 1, 2, 3 or 4 substituents independently selected from R¹¹. In one variation, each R^{c} and R^{d} is independently hydrogen or C₁₋₆ alkyl.

In some embodiments, each R^{c} is independently C₁₋₆ alkyl, C₃₋₈ cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of Rc are each optionally substituted with 1, 2, 3 or 4 substituents independently selected from R¹¹. In one variation, R^{c} is independently C₁₋₆ alkyl.

In some embodiments, each R¹¹ is independently hydrogen or C₁₋₆ alkyl. In one variation, R^{f2} is hydrogen.

In some embodiments, each R¹¹ is independently oxo, C₁₋₆ alkyl, C₂₋₆ alkenyl, C₂₋₆ alkynyl, C₃₋₆ cycloalkyl, C₆₋₁₀ aryl, 5- to 10-membered heteroaryl, 3- to 8-membered heterocyclyl, halogen, cyano, —C(O)R^{a1}, —C(O)OR^{b1}, —C(O)NR^{c1}R^{d1}, —OR^{b1}, —OC(O)R^{a1}, —OC(O)NR^{c1}R^{d1}, —SR^{b1}, —S(O)R^{c1}, —S(O)₂R^{c1}, —S(O)₂NR^{c1}R^{d1}, —NR^{c1}R^{d1}, —N(R^{f1})C(O)R^{a1}, —N(R^{f1})C(O)OR^{b1}, —N(R^{f1})C(O)NR^{c1}R^{d1}, —N(R^{f1})S(O)₂R^{c1}, or —N(R^{f1})S(O)₂NR^{c1}R^{d1}; wherein the C₁₋₆ alkyl, C₂₋₆ alkenyl, C₂₋₆ alkynyl, C₃₋₆ cycloalkyl, C₆₋₁₀ aryl, 5- to 10-membered heteroaryl and 3- to 8-membered heterocyclyl of R¹¹ are each optionally substituted with 1, 2, 3 or 4 substituents independently selected from R¹².

In one variation, each R¹¹ is independently oxo, C₁₋₆ alkyl, C₃₋₆ cycloalkyl, 3- to 8-membered heterocyclyl, halogen, cyano, or —OR^{b1}; wherein the C₁₋₆ alkyl, C₃₋₆ cycloalkyl, and 3- to 8-membered heterocyclyl of R¹¹ are each optionally substituted with 1, 2, 3 or 4 substituents independently selected from R¹².

In one variation, R¹¹ is C₁₋₆ alkyl optionally substituted with 1, 2, 3 or 4 substituents independently selected from R¹². In one variation, R¹¹ is 3- to 8-membered heterocyclyl optionally substituted with 1, 2, 3 or 4 substituents independently selected from R¹².

In one variation, R¹¹ is halogen, cyano, —NR^{c1}R^{d1}, —C(O)NR^{c1}R^{d1}, —OR^{b1}, —S(O)₂R^{c}, C₁₋₆ haloalkyl, —(C₁₋₆ alkylene)-OH, or —(C₁₋₆ alkylene)—NH₂.

In one variation, R¹¹ is hydroxl, cyano, halogen, —CHF₂, —CF₃, —NH₂, —NH(C₁₋₆ alkyl), —N(C₁₋₆ alkyl)₂, —O(C₁₋₆ alkyl), —SO₂(C₁₋₆ alkyl), —S(O)₂NR^{c1}R^{d1}, —C(O)NR^{c1}R^{d1}, or —N(R^{f1})C(O)R^{a1}.

In one variation, R¹¹ is halogen, cyano, —O(C₁₋₆ alkyl), —O(C₁₋₆ alkylene)—NH₂, or —(C₁₋₆ alkylene)-OH.

In some embodiments, each R^{a1} is independently hydrogen, C₁₋₆ alkyl, C₂₋₆ alkenyl, C₂₋₆ alkynyl, C₃₋₆ cycloalkyl, C₆₋₁₀ aryl, 5- to 10-membered heteroaryl or 3- to 8-membered heterocyclyl; wherein the C₁₋₆ alkyl, C₂₋₆ alkenyl, C₂₋₆ alkynyl, C₃₋₆ cycloalkyl, C₆₋₁₀ aryl, 5- to 10-membered heteroaryl and 3- to 8-membered heterocyclyl of R^{a1} are each optionally substituted with 1, 2, 3 or 4 substituents independently selected from R¹².

In some embodiments, each R^{b1} is independently hydrogen, C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{b1} are each optionally substituted with 1, 2, 3 or 4 substituents independently selected from R¹². In one variation, R^{b1} is independently hydrogen or C₁₋₆ alkyl.

In some embodiments, each R^{c1} and R^{d1} is independently hydrogen, C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{c1} and R^{d1} are each optionally substituted with 1, 2, 3 or 4 substituents independently selected from R¹²; or R^{c1} and R^{d1} are taken together with the nitrogen atom to which they are attached to form a 4- to 8-membered heterocyclyl optionally substituted with 1, 2, 3 or 4 substituents independently selected from R¹². In one variation, each R^{c1} and R^{d1} is independently hydrogen or C₁₋₆ alkyl.

In some embodiments, each R^{c1} is independently C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{c1} are each optionally substituted with 1, 2, 3 or 4 substituents independently selected from R¹². In one variation, R^{c1} is independently C₁₋₆ alkyl.

In some embodiments, each R^{f2} is independently hydrogen or C₁₋₆ alkyl. In one variation, R^{f2} is hydrogen.

In some embodiments, each R¹² is independently oxo, C₁₋₆ alkyl, C₃₋₆ cycloalkyl, C₆ aryl, 5- to 6-membered heteroaryl, 3- to 6-membered heterocyclyl, halogen, cyano, —C(O)R^{a2}, —C(O)OR^{b2}, —C(O)NR^{c2}R^{d2}, —OR^{b2}, —OC(O)R^{a2}, —OC(O)NR^{c2}R^{d2}, —S(O)₂R^{c2}, —S(O)₂NR^{c2}R^{d2}, —NR^{c2}R^{d2}, —N(R^{f})C(O)R^{a2}, —N(R^{f})C(O)OR^{b2}, —N(R^{f})C(O)NR^{c2}R^{d2}, —N(R²)S(O)₂R^{c2}, or —N(R^{f2})S(O)₂NR^{c2}R^{d2}; wherein the C₁₋₆ alkyl, C₃₋₆ cycloalkyl, C₆ aryl, 5- to 6-membered heteroaryl and 3- to 6-membered heterocyclyl of R¹² are each optionally substituted with 1, 2, 3 or 4 substituents independently selected from R¹³.

In one variation, each R¹² is independently oxo, halogen, cyano, —OR^{b2}, or C₁₋₆ alkyl optionally substituted with 1, 2, 3 or 4 substituents independently selected from R¹³. In one variation, each R¹² is independently oxo, halogen, cyano, or hydroxyl.

In one variation, R¹² is C₁₋₆ alkyl optionally substituted with 1, 2, 3 or 4 substituents independently selected from R¹³.

In one variation, R¹² is oxo, hydroxyl, C₁₋₆ alkyl, or —O(C₁₋₆ alkyl).

In some embodiments, each R^{a2} is independently hydrogen, C₁₋₆ alkyl, C₃₋₆ cycloalkyl, C₆ aryl, 5- to 6-membered heteroaryl or 3- to 6-membered heterocyclyl; wherein the C₁₋₆ alkyl, C₃₋₆ cycloalkyl, C₆ aryl, 5- to 6-membered heteroaryl and 3- to 6-membered heterocyclyl of R^{a2} are each optionally substituted with 1, 2, 3 or 4 substituents independently selected from R¹³. In one variation, R^{a2} is independently hydrogen or C₁₋₆ alkyl.

In some embodiments, each R^{b2} is independently hydrogen, C₁₋₆ alkyl, C₃₋₆ cycloalkyl or 3- to 6-membered heterocyclyl; wherein the C₁₋₆ alkyl, C₃₋₆ cycloalkyl and 3- to 6-membered heterocyclyl of R^{b2} are each optionally substituted with 1, 2, 3 or 4 substituents independently selected from R¹³. In one variation, R^{b2} is hydrogen.

In some embodiments, each R^{c2} and R¹² is independently hydrogen, C₁₋₆ alkyl, C₃₋₆ cycloalkyl or 3- to 8-membered heterocyclyl; wherein the C₁₋₆ alkyl, C₃₋₆ cycloalkyl and 3- to 8-membered heterocyclyl of R^{c2} and R^{d2} are each optionally substituted with 1, 2, 3 or 4 substituents independently selected from R¹³; or R^{c2} and R^{d2} are taken together with the nitrogen atom to which they are attached to form a 4- to 6-membered heterocyclyl optionally substituted with 1, 2, 3 or 4 substituents independently selected from R¹³. In one variation, each R^{c2} and R^{d2} is independently hydrogen or C₁₋₆ alkyl.

In some embodiments, each R^{c2} is independently C₁₋₆ alkyl, C₃₋₆ cycloalkyl, C₆ aryl, 5- to 6-membered heteroaryl or 3- to 6-membered heterocyclyl; wherein the C₁₋₆ alkyl, C₃₋₆ cycloalkyl, C₆ aryl, 5- to 6-membered heteroaryl and 3- to 6-membered heterocyclyl of R^{c2} are each optionally substituted with 1, 2, 3 or 4 substituents independently selected from R¹³. In one variation, R^{c2} is independently C₁₋₆ alkyl.

In some embodiments, each R^{f2} is independently hydrogen or C₁₋₆ alkyl. In one variation, R^{f2} is hydrogen.

In some embodiments, each R¹³ is independently oxo, halogen, hydroxyl, —O(C₁₋₆ alkyl), cyano, C₁₋₆ alkyl or C₁₋₆ haloalkyl.

In one variation, each R¹³ is independently halogen, hydroxyl, —O(C₁₋₆ alkyl), cyano, or C₁₋₆ alkyl.

In one variation, R¹³ is oxo, hydroxyl, C₁₋₆ alkyl, or —O(C₁₋₆ alkyl).

Representative compounds are listed in Table 1. In some instances, the enantiomers or diastereomers are identified by their respective properties, for example, their relative retention times on a chiral HPLC/SFC or its biological activities, and the absolute stereo configurations of the chiral centers are arbitrarily assigned.

TABLE 1

| No. | Structure | Name |
|---|---|---|
| 101 | 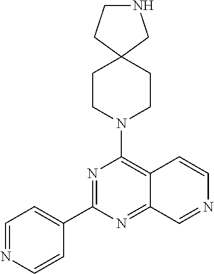 [SMILES: c1cc(-c2nc(N3CCC4(CCNC4)CC3)c3ccncc3n2)ccn1] | 2-(pyridin-4-yl)-4-(2,8- diazaspiro[4.5]decan-8-yl)pyrido[3,4- d]pyrimidine |
| 102 | 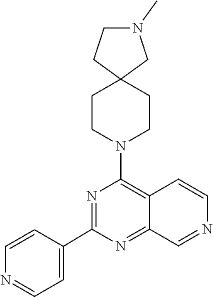 [SMILES: CN1CCC2(CCN(c3nc(-c4ccncc4)nc4cnccc34)CC2)C1] | 4-(2-methyl-2,8-diazaspiro[4.5]decan-8- yl)-2-(pyridin-4-yl)pyrido[3,4- d]pyrimidine |
| 103/ 104 | 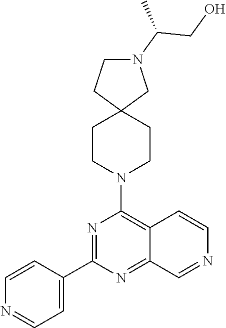 [SMILES: C[C@H](CO)N1CCC2(CCN(c3nc(-c4ccncc4)nc4cnccc34)CC2)C1] | 2-(8-(2-(pyridin-4-yl)pyrido[3,4- d]pyrimidin-4-yl)-2,8- diazaspiro[4.5]decan-2-yl)propan-1-ol (R)-2-(8-(2-(pyridin-4-yl)pyrido[3,4- d]pyrimidin-4-yl)-2,8- diazaspiro[4.5]decan-2-yl)propan-1-ol (S)-2-(8-(2-(pyridin-4-yl)pyrido[3,4- d\|pyrimidin-4-yl)-2,8- diazaspiro[4.5]decan-2-yl)propan-1-ol |
| | 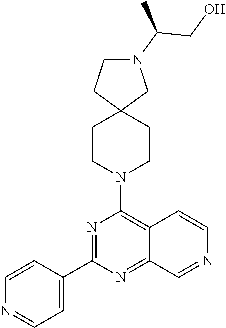 [SMILES: C[C@@H](CO)N1CCC2(CCN(c3nc(-c4ccncc4)nc4cnccc34)CC2)C1] | |
| 105 | 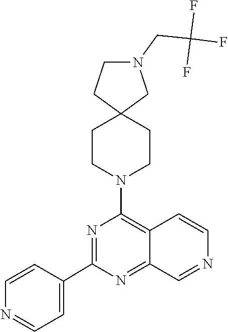 [SMILES: FC(F)(F)CN1CCC2(CCN(c3nc(-c4ccncc4)nc4cnccc34)CC2)C1] | 2-(pyridin-4-yl)-4-(2-(2,2,2- trifluoroethyl)-2,8-diazaspiro[4.5]decan- 8-yl)pyrido[3,4-d]pyrimidine |
| 106 | 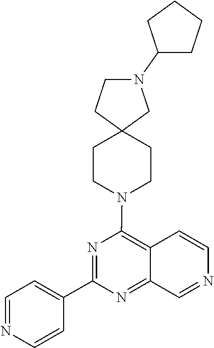 [SMILES: c1cc(-c2nc(N3CCC4(CC3)CCN(C3CCCC3)C4)c3ccncc3n2)ccn1] | 4-(2-cyclopentyl-2,8- diazaspiro[4.5]decan-8-yl)-2-(pyridin-4- yl)pyrido[3,4-d]pyrimidine |
| 107 | 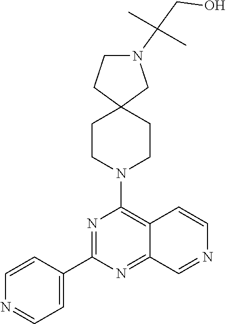 [SMILES: CC(C)(CO)N1CCC2(CCN(c3nc(-c4ccncc4)nc4cnccc34)CC2)C1] | 2-methyl-2-(8-(2-(pyridin-4- yl)pyrido[3,4-d]pyrimidin-4-yl)-2,8- diazaspiro[4.5]decan-2-yl)propan-1-ol |
| 108 | 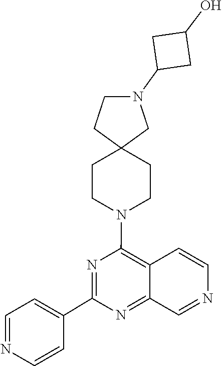 [SMILES: OC1CC(N2CCC3(CCN(c4nc(-c5ccncc5)nc5cnccc45)CC3)C2)C1] | 3-(8-(2-(pyridin-4-yl)pyrido[3,4- d\|pyrimidin-4-yl)-2,8- diazaspiro[4.5]decan-2-yl)cyclobutan-1- ol |
| 109 | 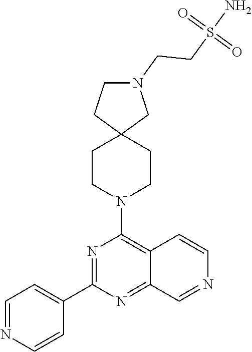 [SMILES: NS(=O)(=O)CCN1CCC2(CCN(c3nc(-c4ccncc4)nc4cnccc34)CC2)C1] | 2-(8-(2-(pyridin-4-yl)pyrido[3,4- d]pyrimidin-4-yl)-2,8- diazaspiro[4.5]decan-2-yl)ethane-1- sulfonamide |
| 110 | 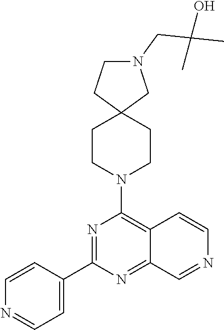 [SMILES: CC(C)(O)CN1CCC2(CCN(c3nc(-c4ccncc4)nc4cnccc34)CC2)C1] | 2-methyl-1-(8-(2-(pyridin-4- yl)pyrido[3,4-d]pyrimidin-4-yl)-2,8- diazaspiro[4.5]decan-2-yl)propan-2-ol |
| 111 | 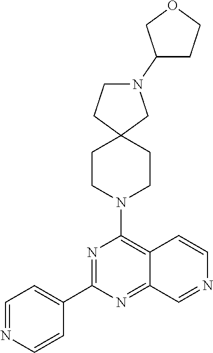 [SMILES: c1cc(-c2nc(N3CCC4(CC3)CCN(C3CCOC3)C4)c3ccncc3n2)ccn1] | 2-(pyridin-4-yl)-4-(2-(tetrahydrofuran-3- yl)-2,8-diazaspiro[4.5]decan-8- yl)pyrido[3,4-d]pyrimidine |
| 112/ 113 | 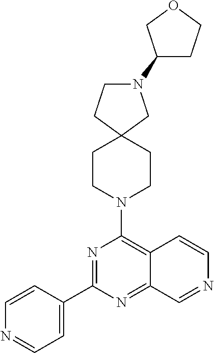 [SMILES: c1cc(-c2nc(N3CCC4(CC3)CCN([C@@H]3CCOC3)C4)c3ccncc3n2)ccn1] | 2-(pyridin-4-yl)-4-(2-(tetrahydrofuran-3- yl)-2,8-diazaspiro[4.5]decan-8- yl)pyrido[3,4-d]pyrimidine (R)-2-(pyridin-4-yl)-4-(2- (tetrahydrofuran-3-yl)-2,8- diazaspiro[4.5]decan-8-yl)pyrido[3,4- d]pyrimidine (S)-2-(pyridin-4-yl)-4-(2- (tetrahydrofuran-3-yl)-2,8- diazaspiro[4.5]decan-8-yl)pyrido[3,4- d]pyrimidine |
| | 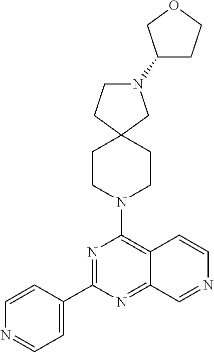 [SMILES: c1cc(-c2nc(N3CCC4(CC3)CCN([C@H]3CCOC3)C4)c3ccncc3n2)ccn1] | |
| 114 | 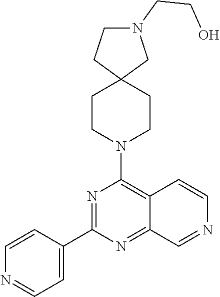 [SMILES: OCCN1CCC2(CCN(c3nc(-c4ccncc4)nc4cnccc34)CC2)C1] | 2-(8-(2-(pyridin-4-yl)pyrido[3,4- d]pyrimidin-4-yl)-2,8- diazaspiro[4.5]decan-2-yl)ethan-1-ol |
| 115/ 116 | 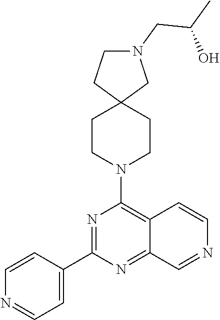 [SMILES: C[C@H](O)CN1CCC2(CCN(c3nc(-c4ccncc4)nc4cnccc34)CC2)C1] | 1-(8-(2-(pyridin-4-yl)pyrido[3,4- d]pyrimidin-4-yl)-2,8- diazaspiro[4.5]decan-2-yl)propan-2-ol (S)-1-(8-(2-(pyridin-4-yl)pyrido[3,4- d]pyrimidin-4-yl)-2,8- diazaspiro[4.5]decan-2-yl)propan-2-ol (R)-1-(8-(2-(pyridin-4-yl)pyrido[3,4- d]pyrimidin-4-yl)-2,8- diazaspiro[4.5]decan-2-yl)propan-2-ol |
| | 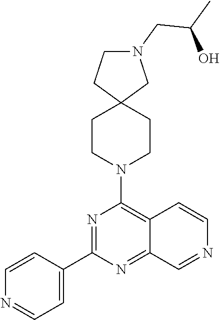 [SMILES: C[C@@H](O)CN1CCC2(CCN(c3nc(-c4ccncc4)nc4cnccc34)CC2)C1] | |
| 117 | 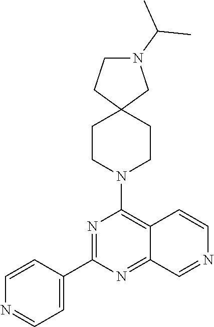 [SMILES: CC(C)N1CCC2(CCN(c3nc(-c4ccncc4)nc4cnccc34)CC2)C1] | 4-(2-isopropyl-2,8-diazaspiro[4.5]decan- 8-yl)-2-(pyridin-4-yl)pyrido[3,4- d]pyrimidine |
| 118 | 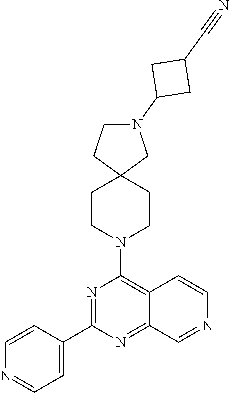 [SMILES: N#CC1CC(N2CCC3(CCN(c4nc(-c5ccncc5)nc5cnccc45)CC3)C2)C1] | 3-(8-(2-(pyridin-4-yl)pyrido[3,4- d]pyrimidin-4-yl)-2,8- diazaspiro[4.5]decan-2-yl)cyclobutane-1- carbonitrile |
| 119 | 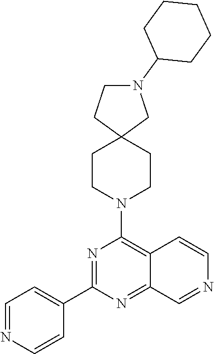 [SMILES: c1cc(-c2nc(N3CCC4(CC3)CCN(C3CCCCC3)C4)c3ccncc3n2)ccn1] | 4-(2-cyclohexyl-2,8- diazaspiro[4.5]decan-8-yl)-2-(pyridin-4- yl)pyrido[3,4-d]pyrimidine |
| 120 | 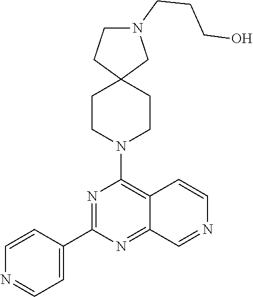 [SMILES: OCCCN1CCC2(CCN(c3nc(-c4ccncc4)nc4cnccc34)CC2)C1] | 3-(8-(2-(pyridin-4-yl)pyrido[3,4- d\|pyrimidin-4-yl)-2,8- diazaspiro[4.5]decan-2-yl)propan-1-ol |
| 121 | 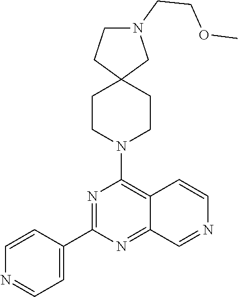 [SMILES: COCCN1CCC2(CCN(c3nc(-c4ccncc4)nc4cnccc34)CC2)C1] | 4-(2-(2-methoxyethyl)-2,8- diazaspiro[4.5]decan-8-yl)-2-(pyridin-4- yl)pyrido[3,4-d]pyrimidine |
| 122 | 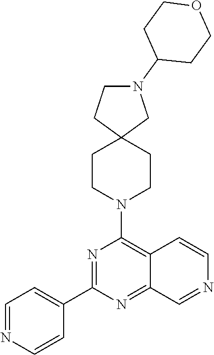 [SMILES: c1cc(-c2nc(N3CCC4(CC3)CCN(C3CCOCC3)C4)c3ccncc3n2)ccn1] | 2-(pyridin-4-yl)-4-(2-(tetrahydro-2H- pyran-4-yl)-2,8-diazaspiro[4.5]decan-8- yl)pyrido[3,4-d]pyrimidine |
| 123 | 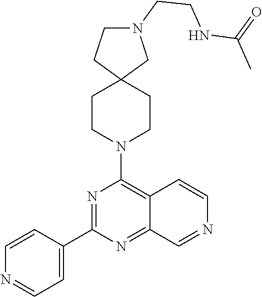 [SMILES: CC(=O)NCCN1CCC2(CCN(c3nc(-c4ccncc4)nc4cnccc34)CC2)C1] | N-(2-(8-(2-(pyridin-4-yl)pyrido[3,4- d]pyrimidin-4-yl)-2,8- diazaspiro[4.5]decan-2- yl)ethyl)acetamide |
| 124 | 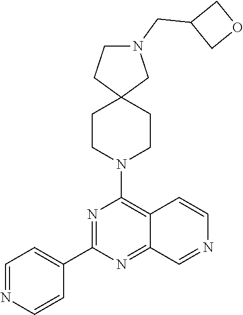 [SMILES: c1cc(-c2nc(N3CCC4(CCN(CC5COC5)C4)CC3)c3ccncc3n2)ccn1] - | 4-(2-(oxetan-3-ylmethyl)-2,8- diazaspiro[4.5]decan-8-yl)-2-(pyridin-4- yl)pyrido[3,4-d]pyrimidine |
| 125 | 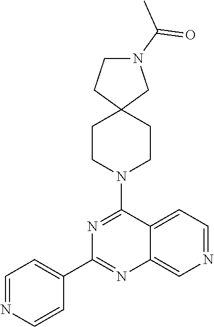 [SMILES: CC(=O)N1CCC2(CCN(c3nc(-c4ccncc4)nc4cnccc34)CC2)C1] | 1-(8-(2-(pyridin-4-yl)pyrido[3,4- d]pyrimidin-4-yl)-2,8- diazaspiro[4.5]decan-2-yl)ethan-1-one |
| 126 | 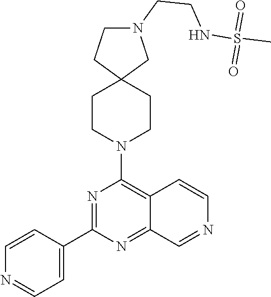 [SMILES: CS(=O)(=O)NCCN1CCC2(CCN(c3nc(-c4ccncc4)nc4cnccc34)CC2)C1] | N-(2-(8-(2-(pyridin-4-yl)pyrido[3,4- d]pyrimidin-4-yl)-2,8- diazaspiro[4.5]decan-2- yl)ethyl)methanesulfonamide |
| 127 | 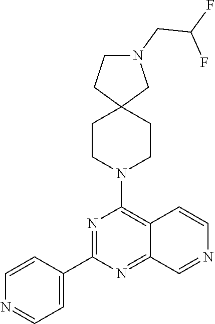 [SMILES: FC(F)CN1CCC2(CCN(c3nc(-c4ccncc4)nc4cnccc34)CC2)C1] | 4-(2-(2,2-difluoroethyl)-2,8- diazaspiro[4.5]decan-8-yl)-2-(pyridin-4- yl)pyrido[3,4-d]pyrimidine |
| 128/ 129 | 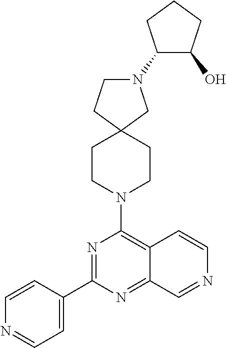 [SMILES: O[C@@H]1CCC[C@H]1N1CCC2(CCN(c3nc(-c4ccncc4)nc4cnccc34)CC2)C1] | (trans)-2-(8-(2-(pyridin-4-yl)pyrido[3,4- d]pyrimidin-4-yl)-2,8- diazaspiro[4.5]decan-2-yl)cyclopentan-1- ol (1R,2R)-2-(8-(2-(pyridin-4-yl)pyrido[3,4- d]pyrimidin-4-yl)-2,8- diazaspiro[4.5]decan-2-yl)cyclopentan-1- ol (1S,2S)-2-(8-(2-(pyridin-4-yl)pyrido[3,4- d]pyrimidin-4-yl)-2,8- diazaspiro[4.5]decan-2-yl)cyclopentan-1- ol |
| | 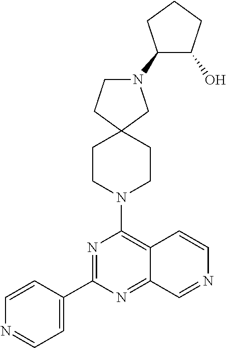 [SMILES: O[C@H]1CCC[C@@H]1N1CCC2(CCN(c3nc(-c4ccncc4)nc4cnccc34)CC2)C1] | |
| 130 | 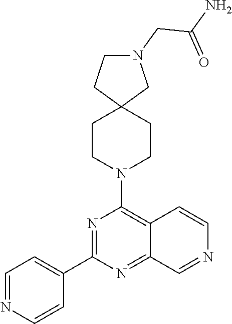 [SMILES: NC(=O)CN1CCC2(CCN(c3nc(-c4ccncc4)nc4cnccc34)CC2)C1] | 2-(8-(2-(pyridin-4-yl)pyrido[3,4- d]pyrimidin-4-yl)-2,8- diazaspiro[4.5]decan-2-yl)acetamide |
| 131 | 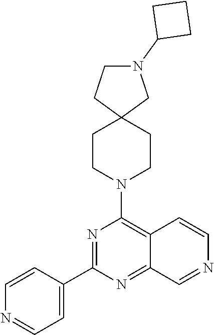 [SMILES: c1cc(-c2nc(N3CCC4(CC3)CCN(C3CCC3)C4)c3ccncc3n2)ccn1] | 4-(2-cyclobutyl-2,8-diazaspiro[4.5]decan- 8-yl)-2-(pyridin-4-yl)pyrido[3,4- d]pyrimidine |
| 132 | 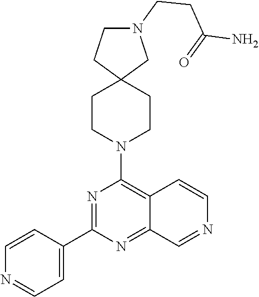 [SMILES: NC(=O)CCN1CCC2(CCN(c3nc(-c4ccncc4)nc4cnccc34)CC2)C1] | 3-(8-(2-(pyridin-4-yl)pyrido[3,4- d]pyrimidin-4-yl)-2,8- diazaspiro[4.5]decan-2-yl)propanamide |
| 133 | 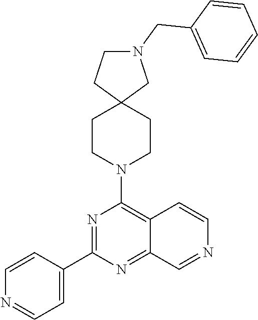 [SMILES: c1ccc(CN2CCC3(CCN(c4nc(-c5ccncc5)nc5cnccc45)CC3)C2)cc1] | 4-(2-benzyl-2,8-diazaspiro[4.5]decan-8- yl)-2-(pyridin-4-yl)pyrido[3,4- d]pyrimidine |
| 134 | 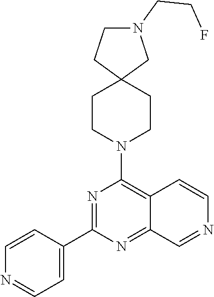 [SMILES: FCCN1CCC2(CCN(c3nc(-c4ccncc4)nc4cnccc34)CC2)C1] | 4-(2-(2-fluoroethyl)-2,8- diazaspiro[4.5]decan-8-yl)-2-(pyridin-4- yl)pyrido[3,4-d]pyrimidine |
| 135 | 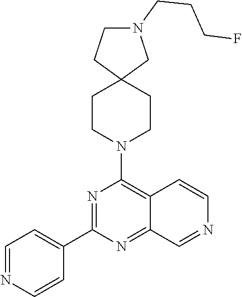 [SMILES: FCCCN1CCC2(CCN(c3nc(-c4ccncc4)nc4cnccc34)CC2)C1] | 4-(2-(3-fluoropropyl)-2,8- diazaspiro[4.5]decan-8-yl)-2-(pyridin-4- yl)pyrido[3,4-d]pyrimidine |
| 136 | 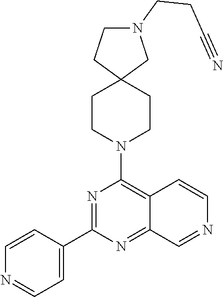 [SMILES: N#CCCN1CCC2(CCN(c3nc(-c4ccncc4)nc4cnccc34)CC2)C1] | 3-(8-(2-(pyridin-4-yl)pyrido[3,4- d]pyrimidin-4-yl)-2,8- diazaspiro[4.5]decan-2-yl)propanenitrile |
| 137 | 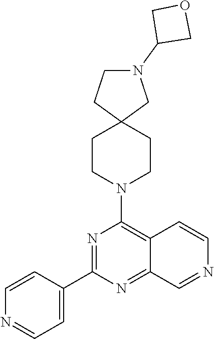 [SMILES: c1cc(-c2nc(N3CCC4(CC3)CCN(C3COC3)C4)c3ccncc3n2)ccn1] | 4-(2-(oxetan-3-yl)-2,8- diazaspiro[4.5]decan-8-yl)-2-(pyridin-4- yl)pyrido[3,4-d]pyrimidine |
| 138 | 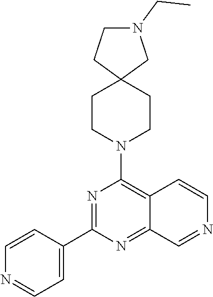 [SMILES: CCN1CCC2(CCN(c3nc(-c4ccncc4)nc4cnccc34)CC2)C1] | 4-(2-ethyl-2,8-diazaspiro[4.5]decan-8-yl)- 2-(pyridin-4-yl)pyrido[3,4-d]pyrimidine |
| 139 | 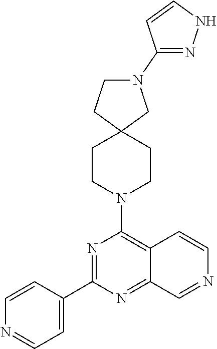 [SMILES: c1cc(-c2nc(N3CCC4(CCN(c5cc[nH]n5)C4)CC3)c3ccncc3n2)ccn1] | 4-(2-(1H-pyrazol-3-yl)-2,8- diazaspiro[4.5]decan-8-yl)-2-(pyridin-4- yl)pyrido[3,4-d]pyrimidine |
| 140 | 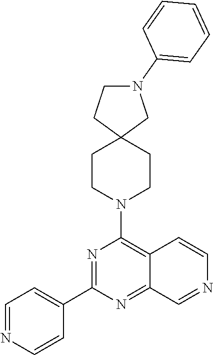 [SMILES: c1ccc(N2CCC3(CCN(c4nc(-c5ccncc5)nc5cnccc45)CC3)C2)cc1] | 4-(2-phenyl-2,8-diazaspiro[4.5]decan-8- yl)-2-(pyridin-4-yl)pyrido[3,4- d]pyrimidine |
| 141 | 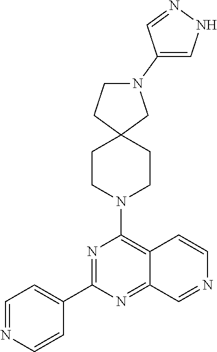 [SMILES: c1cc(-c2nc(N3CCC4(CCN(c5cn[nH]c5)C4)CC3)c3ccncc3n2)ccn1] | 4-(2-(1H-pyrazol-4-yl)-2,8- diazaspiro[4.5]decan-8-yl)-2-(pyridin-4- yl)pyrido[3,4-d]pyrimidine |
| 142 | 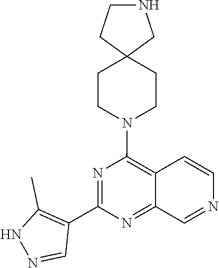 [SMILES: Cc1[nH]ncc1-c1nc(N2CCC3(CCNC3)CC2)c2ccncc2n1] | 2-(5-methyl-1H-pyrazol-4-yl)-4-(2,8- diazaspiro[4.5]decan-8-yl)pyrido[3,4- d]pyrimidine |
| 143 | 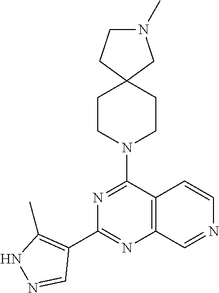 [SMILES: Cc1[nH]ncc1-c1nc(N2CCC3(CCN(C)C3)CC2)c2ccncc2n1] | 2-(5-methyl-1H-pyrazol-4-yl)-4-(2- methyl-2,8-diazaspiro[4.5]decan-8- yl)pyrido[3,4-d]pyrimidine |
| 144/ 145 | 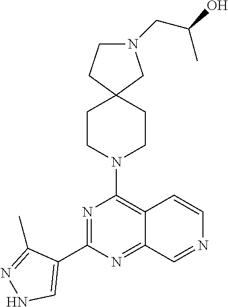 [SMILES: Cc1n[nH]cc1-c1nc(N2CCC3(CCN(C[C@H](C)O)C3)CC2)c2ccncc2n1] | 1-(8-(2-(3-methyl-1H-pyrazol-4- yl)pyrido[3,4-d]pyrimidin-4-yl)-2,8- diazaspiro[4.5]decan-2-yl)propan-2-ol (S)-1-(8-(2-(3-methyl-1H-pyrazol-4- yl)pyrido[3,4-d]pyrimidin-4-yl)-2,8- diazaspiro[4.5]decan-2-yl)propan-2-ol (R)-1-(8-(2-(3-methyl-1H-pyrazol-4- yl)pyrido[3,4-d]pyrimidin-4-yl)-2,8- diazaspiro[4.5]decan-2-yl)propan-2-ol |
| | 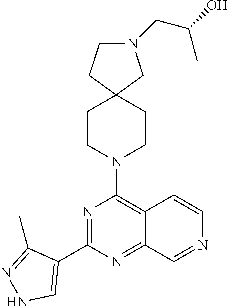 [SMILES: Cc1n[nH]cc1-c1nc(N2CCC3(CCN(C[C@@H](C)O)C3)CC2)c2ccncc2n1] | |
| 146 | 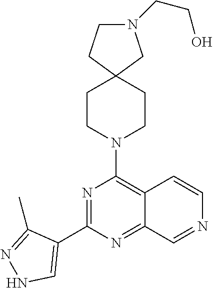 [SMILES: Cc1n[nH]cc1-c1nc(N2CCC3(CCN(CCO)C3)CC2)c2ccncc2n1] | 2-(8-(2-(3-methyl-1H-pyrazol-4- yl)pyrido[3,4-d]pyrimidin-4-yl)-2,8- diazaspiro[4.5]decan-2-yl)ethan-1-ol |
| 147 | 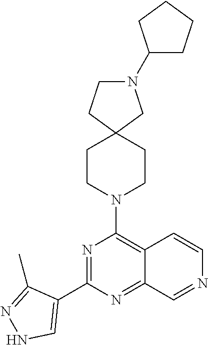 [SMILES: Cc1n[nH]cc1-c1nc(N2CCC3(CC2)CCN(C2CCCC2)C3)c2ccncc2n1] | 4-(2-cyclopentyl-2,8- diazaspiro[4.5]decan-8-yl)-2-(3-methyl- 1H-pyrazol-4-yl)pyrido[3,4-d]pyrimidine |
| 148 | 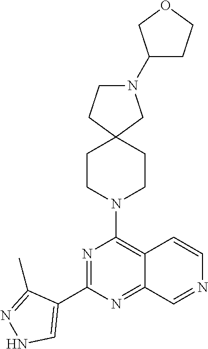 [SMILES: Cc1n[nH]cc1-c1nc(N2CCC3(CC2)CCN(C2CCOC2)C3)c2ccncc2n1] | 2-(3-methyl-1H-pyrazol-4-yl)-4-(2- (tetrahydrofuran-3-yl)-2,8- diazaspiro[4.5]decan-8-yl)pyrido[3,4- d]pyrimidine |
| 149 | 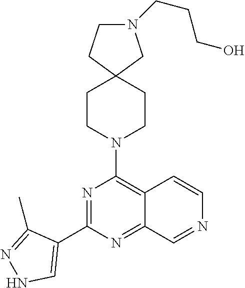 [SMILES: Cc1n[nH]cc1-c1nc(N2CCC3(CCN(CCCO)C3)CC2)c2ccncc2n1] | 3-(8-(2-(3-methyl-1H-pyrazol-4- yl)pyrido[3,4-d]pyrimidin-4-yl)-2,8- diazaspiro[4.5]decan-2-yl)propan-1-ol |
| 150 | 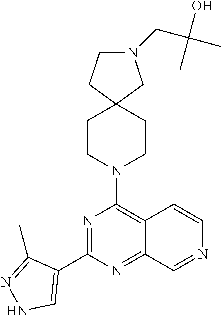 [SMILES: Cc1n[nH]cc1-c1nc(N2CCC3(CCN(CC(C)(C)O)C3)CC2)c2ccncc2n1] | 2-methyl-1-(8-(2-(3-methyl-1H-pyrazol- 4-yl)pyrido[3,4-d]pyrimidin-4-yl)-2,8- diazaspiro[4.5]decan-2-yl)propan-2-ol |
| 151 | 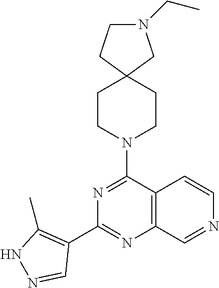 [SMILES: CCN1CCC2(CCN(c3nc(-c4cn[nH]c4C)nc4cnccc34)CC2)C1] | 4-(2-ethyl-2,8-diazaspiro[4.5]decan-8-yl)- 2-(5-methyl-1H-pyrazol-4-yl)pyrido[3,4- d]pyrimidine |
| 152 | 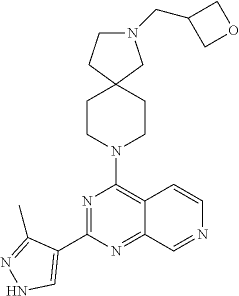 [SMILES: Cc1n[nH]cc1-c1nc(N2CCC3(CCN(CC4COC4)C3)CC2)c2ccncc2n1] | 2-(3-methyl-1H-pyrazol-4-yl)-4-(2- (oxetan-3-ylmethyl)-2,8- diazaspiro[4.5]decan-8-yl)pyrido[3,4- d]pyrimidine |
| 153 | 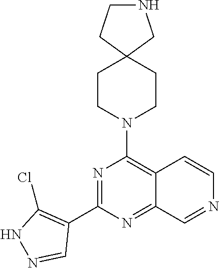 [SMILES: Clc1[nH]ncc1-c1nc(N2CCC3(CCNC3)CC2)c2ccncc2n1] | 2-(5-chloro-1H-pyrazol-4-yl)-4-(2,8- diazaspiro[4.5]decan-8-yl)pyrido[3,4- d]pyrimidine |
| 154 | 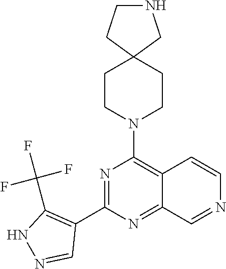 [SMILES: FC(F)(F)c1[nH]ncc1-c1nc(N2CCC3(CCNC3)CC2)c2ccncc2n1] | 4-(2,8-diazaspiro[4.5]decan-8-yl)-2-(5- (trifluoromethyl)-1H-pyrazol-4- yl)pyrido[3,4-d]pyrimidine |
| 155 | 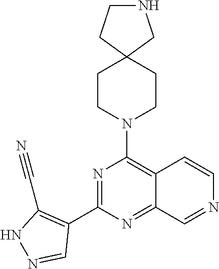 [SMILES: N#Cc1[nH]ncc1-c1nc(N2CCC3(CCNC3)CC2)c2ccncc2n1] | 4-(4-(2,8-diazaspiro[4.5]decan-8- yl)pyrido[3,4-d]pyrimidin-2-yl)-1H- pyrazole-5-carbonitrile |
| 156 | 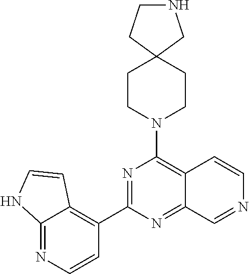 [SMILES: c1cc2c(N3CCC4(CCNC4)CC3)nc(-c3ccnc4[nH]ccc34)nc2cn1] | 2-(1H-pyrrolo[2,3-b]pyridin-4-yl)-4-(2,8- diazaspiro[4.5]decan-8-yl)pyrido[3,4- d]pyrimidine |
| 157 | 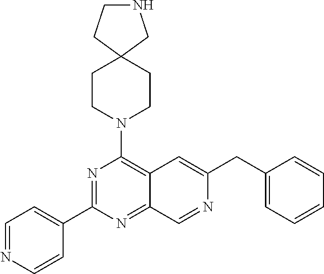 [SMILES: c1ccc(Cc2cc3c(N4CCC5(CCNC5)CC4)nc(-c4ccncc4)nc3cn2)cc1] | 6-benzyl-2-(pyridin-4-yl)-4-(2,8- diazaspiro[4.5]decan-8-yl)pyrido[3,4- d]pyrimidine |
| 158 | 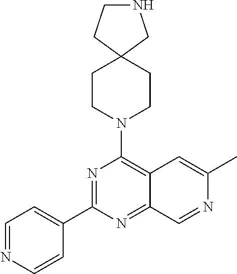 [SMILES: Cc1cc2c(N3CCC4(CCNC4)CC3)nc(-c3ccncc3)nc2cn1] | 6-methyl-2-(pyridin-4-yl)-4-(2,8- diazaspiro[4.5]decan-8-yl)pyrido[3,4- d]pyrimidine |
| 159 | 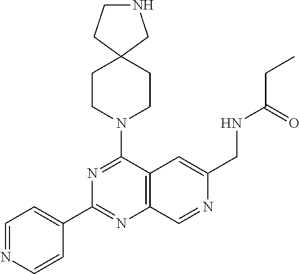 [SMILES: CCC(=O)NCc1cc2c(N3CCC4(CCNC4)CC3)nc(-c3ccncc3)nc2cn1] | N-((2-(pyridin-4-yl)-4-(2,8- diazaspiro[4.5]decan-8-yl)pyrido[3,4- d]pyrimidin-6-yl)methyl)propionamide |
| 160 | 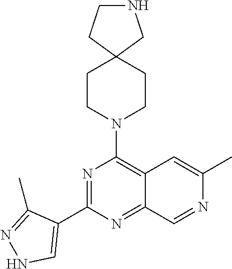 [SMILES: Cc1cc2c(N3CCC4(CCNC4)CC3)nc(-c3c[nH]nc3C)nc2cn1] | 6-methyl-2-(3-methyl-1H-pyrazol-4-yl)- 4-(2,8-diazaspiro[4.5]decan-8- yl)pyrido[3,4-d]pyrimidine |
| 161 | 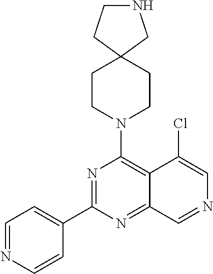 [SMILES: Clc1cncc2nc(-c3ccncc3)nc(N3CCC4(CCNC4)CC3)c12] | 5-chloro-2-(pyridin-4-yl)-4-(2,8- diazaspiro[4.5]decan-8-yl)pyrido[3,4- d]pyrimidine |
| 162 | 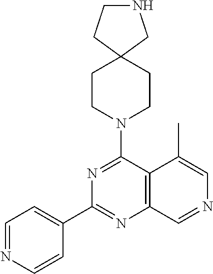 [SMILES: Cc1cncc2nc(-c3ccncc3)nc(N3CCC4(CCNC4)CC3)c12] | 5-methyl-2-(pyridin-4-yl)-4-(2,8- diazaspiro[4.5]decan-8-yl)pyrido[3,4- d]pyrimidine |
| 163 | 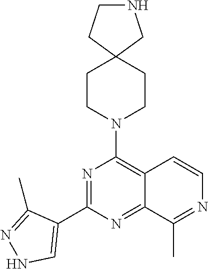 [SMILES: Cc1n[nH]cc1-c1nc(N2CCC3(CCNC3)CC2)c2ccnc(C)c2n1] | 8-methyl-2-(3-methyl-1H-pyrazol-4-yl)- 4-(2,8-diazaspiro[4.5]decan-8- yl)pyrido[3,4-d]pyrimidine |
| 164 | 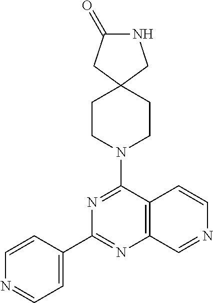 [SMILES: O=C1CC2(CCN(c3nc(-c4ccncc4)nc4cnccc34)CC2)CN1] | 8-(2-(pyridin-4-yl)pyrido[3,4- d]pyrimidin-4-yl)-2,8- diazaspiro[4.5]decan-3-one |
| 165 | 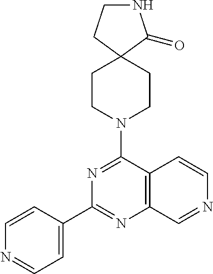 [SMILES: O=C1NCCC12CCN(c1nc(-c3ccncc3)nc3cnccc13)CC2] | 8-(2-(pyridin-4-yl)pyrido[3,4- d]pyrimidin-4-yl)-2,8- diazaspiro[4.5]decan-1-one |
| 166 | 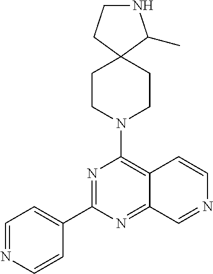 [SMILES: CC1NCCC12CCN(c1nc(-c3ccncc3)nc3cnccc13)CC2] | 4-(1-methyl-2,8-diazaspiro[4.5]decan-8- yl)-2-(pyridin-4-yl)pyrido[3,4- d]pyrimidine |
| 167 | 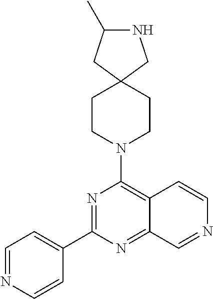 [SMILES: CC1CC2(CCN(c3nc(-c4ccncc4)nc4cnccc34)CC2)CN1] | 4-(3-methyl-2,8-diazaspiro[4.5]decan-8- yl)-2-(pyrdin-4-yl)pyrido[3,4- d]pyrimidine |
| 168 | 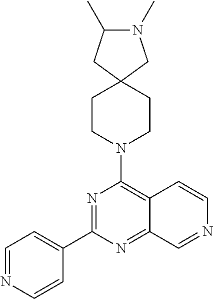 [SMILES: CC1CC2(CCN(c3nc(-c4ccncc4)nc4cnccc34)CC2)CN1C] | 4-(2,3-dimethyl-2,8-diazaspiro[4.5]decan- 8-yl)-2-(pyridin-4-yl)pyrido[3,4- d]pyrimidine |
| 169/ 170 | 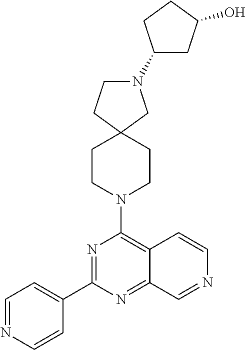 [SMILES: O[C@H]1CC[C@@H](N2CCC3(CCN(c4nc(-c5ccncc5)nc5cnccc45)CC3)C2)C1] | (cis)-3-(8-(2-(pyridin-4-yl)pyrido[3,4- d]pyrimidin-4-yl)-2,8- diazaspiro[4.5]decan-2-yl)cyclopentan-1- ol (1S,3R)-3-(8-(2-(pyridin-4-yl)pyrido[3,4- d]pyrimidin-4-yl)-2,8- diazaspiro[4.5]decan-2-yl)cyclopentan-1- ol (1R,3S)-3-(8-(2-(pyridin-4-yl)pyrido[3,4- d]pyrimidin-4-yl)-2,8- diazaspiro[4.5]decan-2-yl)cyclopentan-1- ol |
| | 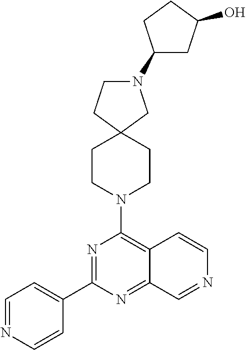 [SMILES: O[C@@H]1CC[C@H](N2CCC3(CCN(c4nc(-c5ccncc5)nc5cnccc45)CC3)C2)C1] | |
| 171 | 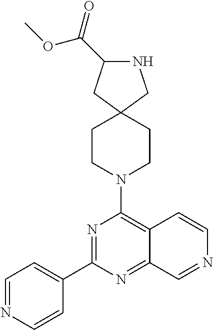 [SMILES: COC(=O)C1CC2(CCN(c3nc(-c4ccncc4)nc4cnccc34)CC2)CN1] | methyl 8-(2-(pyridin-4-yl)pyrido[3,4- d]pyrimidin-4-yl)-2,8- diazaspiro[4.5]decane-3-carboxylate |
| 172 | 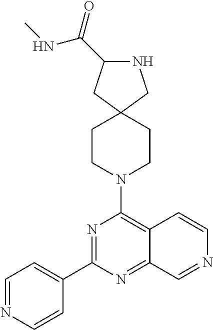 [SMILES: CNC(=O)C1CC2(CCN(c3nc(-c4ccncc4)nc4cnccc34)CC2)CN1] | N-methyl-8-(2-(pyridin-4-yl)pyrido[3,4- d]pyrimidin-4-yl)-2,8- diazaspiro[4.5]decane-3-carboxamide |
| 173 | 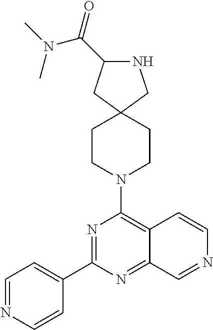 [SMILES: CN(C)C(=O)C1CC2(CCN(c3nc(-c4ccncc4)nc4cnccc34)CC2)CN1] | N,N-dimethyl-8-(2-(pyridin-4- yl)pyrido[3,4-d]pyrimidin-4-yl)-2,8- diazaspiro[4.5]decane-3-carboxamide |
| 174 | 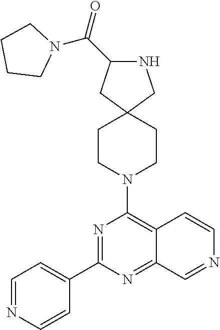 [SMILES: O=C(C1CC2(CCN(c3nc(-c4ccncc4)nc4cnccc34)CC2)CN1)N1CCCC1] | (8-(2-(pyridin-4-yl)pyrido[3,4- d]pyrimidin-4-yl)-2,8- diazaspiro[4.5]decan-3-yl)(pyrrolidin-1- yl)methanone |
| 175 | 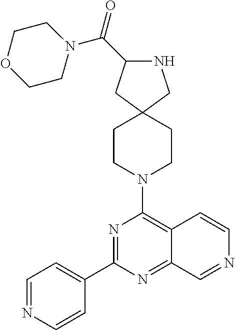 [SMILES: O=C(C1CC2(CCN(c3nc(-c4ccncc4)nc4cnccc34)CC2)CN1)N1CCOCC1] | morpholino(8-(2-(pyridin-4- yl)pyrido[3,4-d]pyrimidin-4-yl)-2,8- diazaspiro[4.5]decan-3-yl)methanone |
| 176/ 177 | 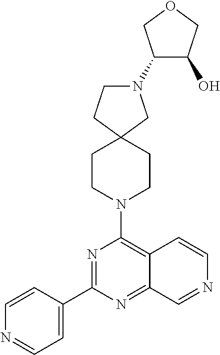 [SMILES: O[C@@H]1COC[C@H]1N1CCC2(CCN(c3nc(-c4ccncc4)nc4cnccc34)CC2)C1] | (trans)-4-(8-(2-(pyridin-4-yl)pyrido[3,4- d]pyrimidin-4-yl)-2,8- diazaspiro[4.5]decan-2- yl)tetrahydrofuran-3-ol (3S,4R)-4-(8-(2-(pyridin-4-yl)pyrido[3,4- d]pyrimidin-4-yl)-2,8- diazaspiro[4.5]decan-2- yl)tetrahydrofuran-3-ol (3R,4S)-4-(8-(2-(pyridin-4-yl)pyrido[3,4- d]pyrimidin-4-yl)-2,8- diazaspiro[4.5]decan-2- yl)tetrahydrofuran-3-ol |
| | 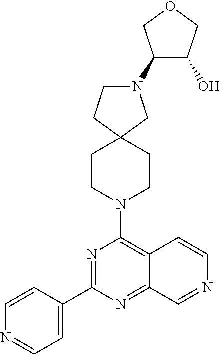 [SMILES: O[C@H]1COC[C@@H]1N1CCC2(CCN(c3nc(-c4ccncc4)nc4cnccc34)CC2)C1] | |
| 178/ 179 | 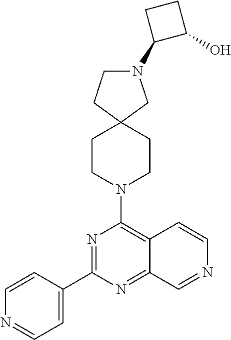 [SMILES: O[C@H]1CC[C@@H]1N1CCC2(CCN(c3nc(-c4ccncc4)nc4cnccc34)CC2)C1] | (trans)-2-(8-(2-(pyridin-4-yl)pyrido[3,4- d]pyrimidin-4-yl)-2,8- diazaspiro[4.5]decan-2-yl)cyclobutan-1- ol (1S,2S)-2-(8-(2-(pyridin-4-yl)pyrido[3,4- d]pyrimidin-4-yl)-2,8- diazaspiro[4.5]decan-2-yl)cyclobutan-1- ol (1R,2R)-2-(8-(2-(pyridin-4-yl)pyrido[3,4- d]pyrimidin-4-yl)-2,8- diazaspiro[4.5]decan-2-yl)cyclobutan-1- ol |
| | 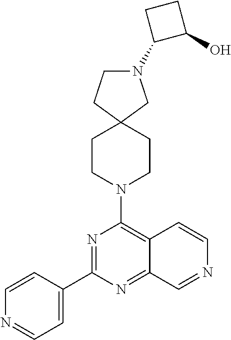 [SMILES: O[C@@H]1CC[C@H]1N1CCC2(CCN(c3nc(-c4ccncc4)nc4cnccc34)CC2)C1] | |
| 180/ 181 | 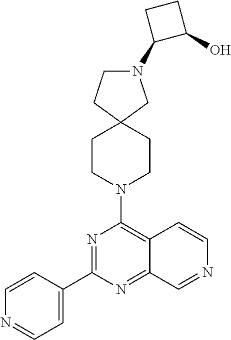 [SMILES: O[C@@H]1CC[C@@H]1N1CCC2(CCN(c3nc(-c4ccncc4)nc4cnccc34)CC2)C1] | (cis)-2-(8-(2-(pyridin-4-yl)pyrido[3,4- d]pyrimidin-4-yl)-2,8- diazaspiro[4.5]decan-2-yl)cyclobutan-1- ol (1R,2S)-2-(8-(2-(pyridin-4-yl)pyrido[3,4- d]pyrimidin-4-yl)-2,8- diazaspiro[4.5]decan-2-yl)cyclobutan-1- ol (1S,2R)-2-(8-(2-(pyridin-4-yl)pyrido[3,4- d]pyrimidin-4-yl)-2,8- diazaspiro[4.5]decan-2-yl)cyclobutan-1- ol |
| | 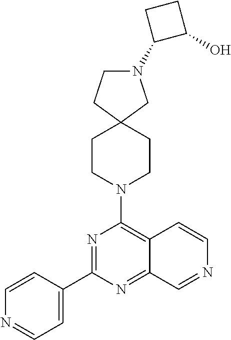 [SMILES: O[C@H]1CC[C@H]1N1CCC2(CCN(c3nc(-c4ccncc4)nc4cnccc34)CC2)C1] | |
| 182 | 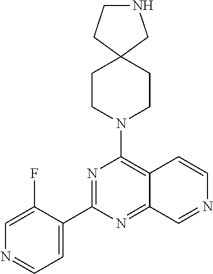 [SMILES: Fc1cnccc1-c1nc(N2CCC3(CCNC3)CC2)c2ccncc2n1] | 2-(3-fluoropyridin-4-yl)-4-(2,8- diazaspiro[4.5]decan-8-yl)pyrido[3,4- d]pyrimidine |
| 183 | 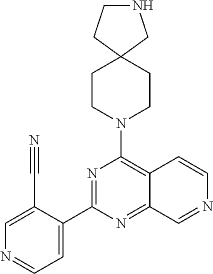 [SMILES: N#Cc1cnccc1-c1nc(N2CCC3(CCNC3)CC2)c2ccncc2n1] | 4-(4-(2,8-diazaspiro[4.5]decan-8- yl)pyrido[3,4-d]pyrimidin-2- yl)nicotinonitrile |
| 184/ 185 | 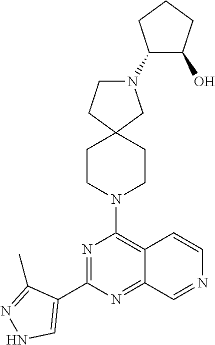 [SMILES: Cc1n[nH]cc1-c1nc(N2CCC3(CC2)CCN([C@@H]2CCC[C@H]2O)C3)c2ccncc2n1] | (trans)-2-(8-(2-(3-methyl-1H-pyrazol-4- yl)pyrido[3,4-d]pyrimidin-4-yl)-2,8- diazaspiro[4.5]decan-2-yl)cyclopentan-1- ol (1R,2R)-2-(8-(2-(3-methyl-1H-pyrazol-4- yl)pyrido[3,4-d]pyrimidin-4-yl)-2,8- diazaspiro[4.5]decan-2-yl)cyclopentan-1- ol (1S,2S)-2-(8-(2-(3-methyl-1H-pyrazol-4- yl)pyrido[3,4-d]pyrimidin-4-yl)-2,8- diazaspiro[4.5]decan-2-yl)cyclopentan-1- ol |
| | 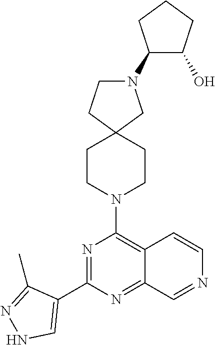 [SMILES: Cc1n[nH]cc1-c1nc(N2CCC3(CC2)CCN([C@H]2CCC[C@@H]2O)C3)c2ccncc2n1] | |
| 186 | 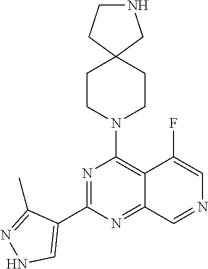 [SMILES: Cc1n[nH]cc1-c1nc(N2CCC3(CCNC3)CC2)c2c(F)cncc2n1] | 5-fluoro-2-(3-methyl-1H-pyrazol-4-yl)-4- (2,8-diazaspiro[4.5]decan-8- yl)pyrido[3,4-d]pyrimidine |
| 187 | 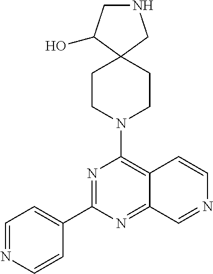 [SMILES: OC1CNCC12CCN(c1nc(-c3ccncc3)nc3cnccc13)CC2] | 8-(2-(pyridin-4-yl)pyrido[3,4- d]pyrimidin-4-yl)-2,8- diazaspiro[4.5]decan-4-ol |
| 188 | 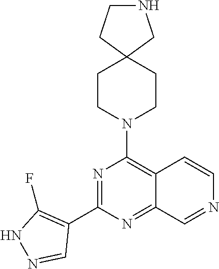 [SMILES: Fc1[nH]ncc1-c1nc(N2CCC3(CCNC3)CC2)c2ccncc2n1] | 2-(5-fluoro-1H-pyrazol-4-yl)-4-(2,8- diazaspiro[4.5]decan-8-yl)pyrido[3,4- d]pyrimidine |
| 189 | 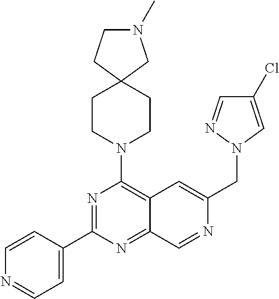 [SMILES: CN1CCC2(CCN(c3nc(-c4ccncc4)nc4cnc(Cn5cc(Cl)cn5)cc34)CC2)C1] | 6-((4-chloro-1H-pyrazol-1-yl)methyl)-4- (2-methyl-2,8-diazaspiro[4.5]decan-8-yl)- 2-(pyridin-4-yl)pyrido[3,4-d]pyrimidine |
| 190 | 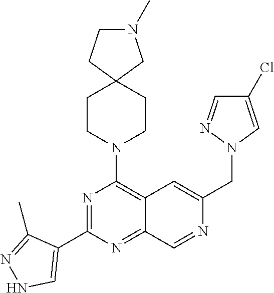 [SMILES: Cc1n[nH]cc1-c1nc(N2CCC3(CCN(C)C3)CC2)c2cc(Cn3cc(Cl)cn3)ncc2n1] | 6-((4-chloro-1H-pyrazol-1-yl)methyl)-2- (3-methyl-1H-pyrazol-4-yl)-4-(2-methyl- 2,8-diazaspiro[4.5]decan-8-yl)pyrido[3,4- d]pyrimidine |
| 191 | 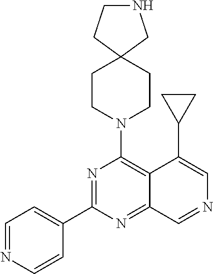 [SMILES: c1cc(-c2nc(N3CCC4(CCNC4)CC3)c3c(C4CC4)cncc3n2)ccn1] | 5-cyclopropyl-2-(pyridin-4-yl)-4-(2,8- diazaspiro[4.5]decan-8-yl)pyrido[3,4- d]pyrimidine |
| 192 | 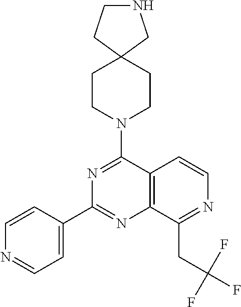 [SMILES: FC(F)(F)Cc1nccc2c(N3CCC4(CCNC4)CC3)nc(-c3ccncc3)nc12] | 2-(pyridin-4-yl)-4-(2,8- diazaspiro[4.5]decan-8-yl)-8-(2,2,2- trifluoroethyl)pyrido[3,4-d]pyrimidine |
| 193 | 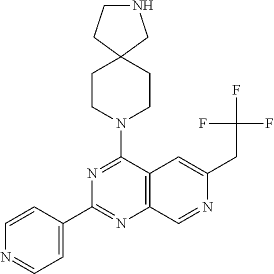 [SMILES: FC(F)(F)Cc1cc2c(N3CCC4(CCNC4)CC3)nc(-c3ccncc3)nc2cn1] | 2-(pyridin-4-yl)-4-(2,8- diazaspiro[4.5]decan-8-yl)-6-(2,2,2- trifluoroethyl)pyrido[3,4-d]pyrimidine |
| 194 | 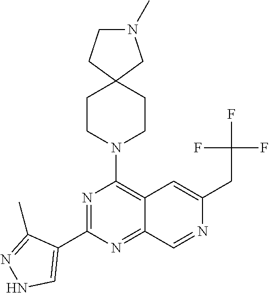 [SMILES: Cc1n[nH]cc1-c1nc(N2CCC3(CCN(C)C3)CC2)c2cc(CC(F)(F)F)ncc2n1] | 2-(3-methyl-1H-pyrazol-4-yl)-4-(2- methyl-2,8-diazaspiro[4.5]decan-8-yl)-6- (2,2,2-trifluoroethyl)pyrido[3,4- d]pyrimidine |
| 195 | 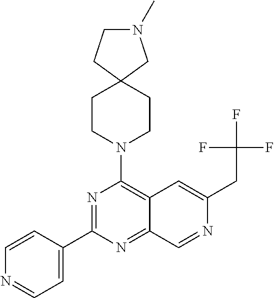 [SMILES: CN1CCC2(CCN(c3nc(-c4ccncc4)nc4cnc(CC(F)(F)F)cc34)CC2)C1] | 4-(2-methyl-2,8-diazaspiro[4.5]decan-8- yl)-2-(pyridin-4-yl)-6-(2,2,2- trifluoroethyl)pyrido[3,4-d]pyrimidine |
| 196 | 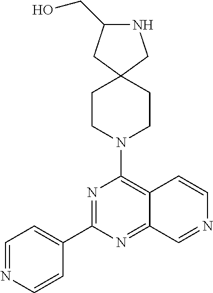 [SMILES: OCC1CC2(CCN(c3nc(-c4ccncc4)nc4cnccc34)CC2)CN1] | (8-(2-(pyridin-4-yl)pyrido[3,4- d]pyrimidin-4-yl)-2,8- diazaspiro[4.5]decan-3-yl)methanol |
| 197/ 198 | 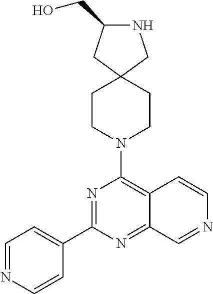 [SMILES: OC[C@@H]1CC2(CCN(c3nc(-c4ccncc4)nc4cnccc34)CC2)CN1] | (8-(2-(pyridin-4-yl)pyrido[3,4- d]pyrimidin-4-yl)-2,8- diazaspiro[4.5]decan-3-yl)methanol (S)-(8-(2-(pyridin-4-yl)pyrido[3,4- d]pyrimidin-4-yl)-2,8- diazaspiro[4.5]decan-3-yl)methanol (R)-(8-(2-(pyridin-4-yl)pyrido[3,4- d]pyrimidin-4-yl)-2,8- diazaspiro[4.5]decan-3-yl)methanol |
| | 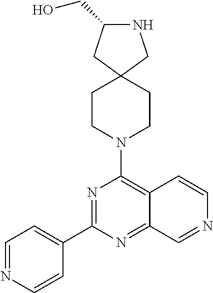 [SMILES: OC[C@H]1CC2(CCN(c3nc(-c4ccncc4)nc4cnccc34)CC2)CN1] | |
| 199 | 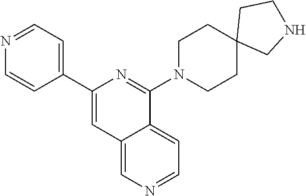 [SMILES: c1cc(-c2cc3cnccc3c(N3CCC4(CCNC4)CC3)n2)ccn1] | 8-(3-(pyridin-4-yl)-2,6-naphthyridin-1- yl)-2,8-diazaspiro[4.5]decane |
| 200 | 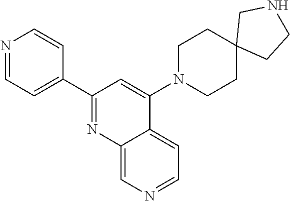 [SMILES: c1cc(-c2cc(N3CCC4(CCNC4)CC3)c3ccncc3n2)ccn1] | 8-(2-(pyridin-4-yl)-1,7-naphthyridin-4- yl)-2,8-diazaspiro[4.5]decane |
| 201 | 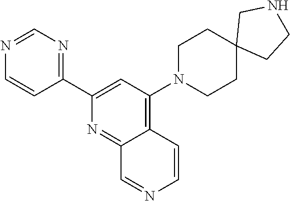 [SMILES: c1cc(-c2cc(N3CCC4(CCNC4)CC3)c3ccncc3n2)ncn1] | 8-(2-(pyrimidin-4-yl)-1,7-naphthyridin-4- yl)-2,8-diazaspiro[4.5]decane |
| 202 | 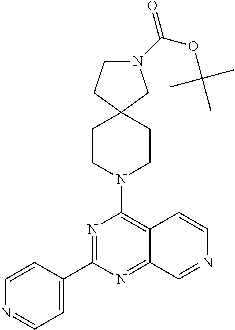 [SMILES: CC(C)(C)OC(=O)N1CCC2(CCN(c3nc(-c4ccncc4)nc4cnccc34)CC2)C1] | tert-butyl 8-(2-(pyridin-4-yl)pyrido[3,4- d]pyrimidin-4-yl)-2,8- diazaspiro[4.5]decane-2-carboxylate |
| 203 | 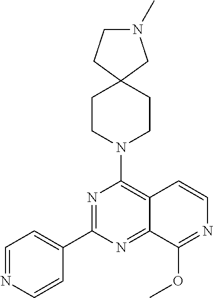 [SMILES: COc1nccc2c(N3CCC4(CCN(C)C4)CC3)nc(-c3ccncc3)nc12] | 8-methoxy-4-(2-methyl-2,8- diazaspiro[4.5]decan-8-yl)-2-(pyridin-4- yl)pyrido[3,4-d]pyrimidine |
| 204 | 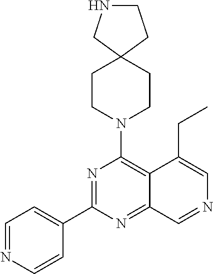 [SMILES: CCc1cncc2nc(-c3ccncc3)nc(N3CCC4(CCNC4)CC3)c12] | 5-ethyl-2-(pyridin-4-yl)-4-(2,8- diazaspiro[4.5]decan-8-yl)pyrido[3,4- d]pyrimidine |
| 205 | 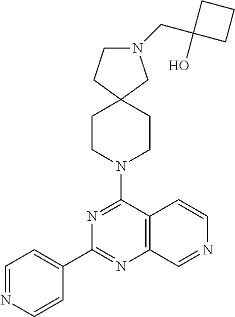 [SMILES: OC1(CN2CCC3(CCN(c4nc(-c5ccncc5)nc5cnccc45)CC3)C2)CCC1] | 1-((8-(2-(pyridin-4-yl)pyrido[3,4- d]pyrimidin-4-yl)-2,8- diazaspiro[4.5]decan-2- yl)methyl)cyclobutan-1-ol |
| 206 | 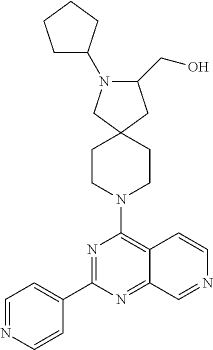 [SMILES: OCC1CC2(CCN(c3nc(-c4ccncc4)nc4cnccc34)CC2)CN1C1CCCC1] | (2-cyclopentyl-8-(2-(pyridin-4- yl)pyrido[3,4-d]pyrimidin-4-yl)-2,8- diazaspiro[4.5]decan-3-yl)methanol |
| 207 | 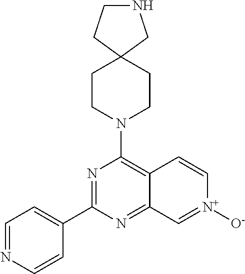 [SMILES: [O-][n+]1ccc2c(N3CCC4(CCNC4)CC3)nc(-c3ccncc3)nc2c1] | 2-(pyridin-4-yl)-4-(2,8- diazaspiro[4.5]decan-8-yl)pyrido[3,4- d]pyrimidine 7-oxide |
| 208 | 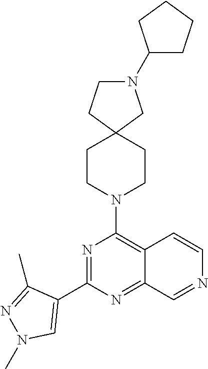 [SMILES: Cc1nn(C)cc1-c1nc(N2CCC3(CC2)CCN(C2CCCC2)C3)c2ccncc2n1] | 4-(2-cyclopentyl-2,8- diazaspiro[4.5]decan-8-yl)-2-(1,3- dimethyl-1H-pyrazol-4-yl)pyrido[3,4- d]pyrimidine |
| 209 | 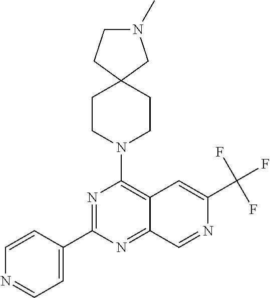 [SMILES: CN1CCC2(CCN(c3nc(-c4ccncc4)nc4cnc(C(F)(F)F)cc34)CC2)C1] | 4-(2-methyl-2,8-diazaspiro[4.5]decan-8- yl)-2-(pyridin-4-yl)-6- (trifluoromethyl)pyrido[3,4-d]pyrimidine |
| 210 | 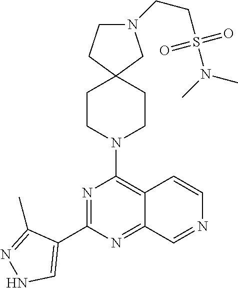 [SMILES: Cc1n[nH]cc1-c1nc(N2CCC3(CCN(CCS(=O)(=O)N(C)C)C3)CC2)c2ccncc2n1] | N,N-dimethyl-2-(8-(2-(3-methyl-1H- pyrazol-4-yl)pyrido[3,4-d]pyrimidin-4- yl)-2,8-diazaspiro[4.5]decan-2-yl)ethane- 1-sulfonamide |
| 211 | 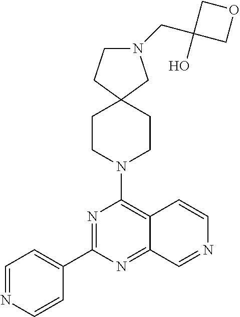 [SMILES: OC1(CN2CCC3(CCN(c4nc(-c5ccncc5)nc5cnccc45)CC3)C2)COC1] | 3-((8-(2-(pyridin-4-yl)pyrido[3,4- d]pyrimidin-4-yl)-2,8- diazaspiro[4.5]decan-2-yl)methyl)oxetan- 3-ol |
| 212 | 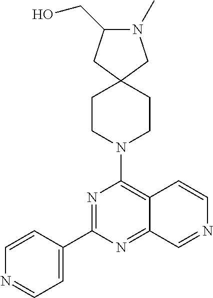 [SMILES: CN1CC2(CCN(c3nc(-c4ccncc4)nc4cnccc34)CC2)CC1CO] | (2-methyl-8-(2-(pyridin-4-yl)pyrido[3,4- d]pyrimidin-4-yl)-2,8- diazaspiro[4.5]decan-3-yl)methanol |
| 213 | 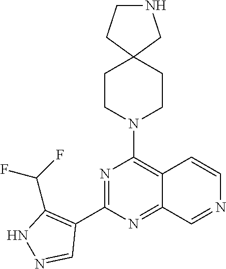 [SMILES: FC(F)c1[nH]ncc1-c1nc(N2CCC3(CCNC3)CC2)c2ccncc2n1] | 2-(5-(difluoromethyl)-1H-pyrazol-4-yl)- 4-(2,8-diazaspiro[4.5]decan-8- yl)pyrido[3,4-d]pyrimidine |
| 214 | 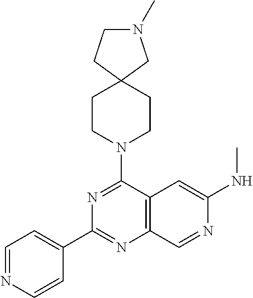 [SMILES: CNc1cc2c(N3CCC4(CCN(C)C4)CC3)nc(-c3ccncc3)nc2cn1] | N-methyl-4-(2-methyl-2,8- diazaspiro[4.5]decan-8-yl)-2-(pyridin-4- yl)pyrido[3,4-d]pyrimidin-6-amine |
| 215 | 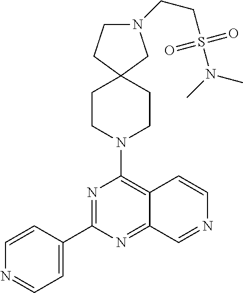 [SMILES: CN(C)S(=O)(=O)CCN1CCC2(CCN(c3nc(-c4ccncc4)nc4cnccc34)CC2)C1] | N,N-dimethyl-2-(8-(2-(pyridin-4- yl)pyrido[3,4-d]pyrimidin-4-yl)-2,8- diazaspiro[4.5]decan-2-yl)ethane-1- sulfonamide |
| 216 | 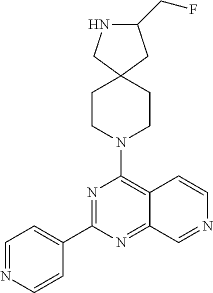 [SMILES: FCC1CC2(CCN(c3nc(-c4ccncc4)nc4cnccc34)CC2)CN1] | 4-(3-(fluoromethyl)-2,8- diazaspiro[4.5]decan-8-yl)-2-(pyridin-4- yl)pyrido[3,4-d]pyrimidine |
| 217 | 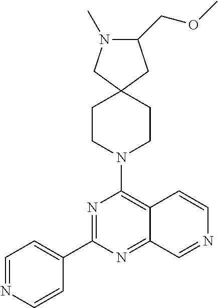 [SMILES: COCC1CC2(CCN(c3nc(-c4ccncc4)nc4cnccc34)CC2)CN1C] | 4-(3-(methoxymethyl)-2-methyl-2,8- diazaspiro[4.5]decan-8-yl)-2-(pyridin-4- yl)pyrido[3,4-d]pyrimidine |
| 218 | 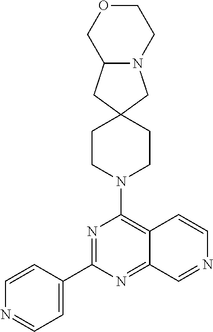 [SMILES: c1cc(-c2nc(N3CCC4(CC3)CC3COCCN3C4)c3ccncc3n2)ccn1] | 1-(2-(pyridin-4-yl)pyrido[3,4- d]pyrimidin-4-yl)tetrahydro-1′H,6′H- spiro[piperidine-4,7′-pyrrolo[2,1- c][1,4]oxazine] |
| 219 | 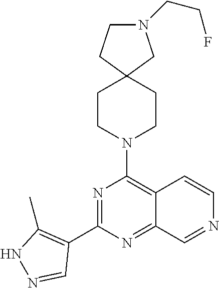 [SMILES: Cc1[nH]ncc1-c1nc(N2CCC3(CCN(CCF)C3)CC2)c2ccncc2n1] | 4-(2-(2-fluoroethyl)-2,8- diazaspiro[4.5]decan-8-yl)-2-(5-methyl- 1H-pyraol-4-yl)pyrido[3,4-d]pyrimidine |
| 220 | 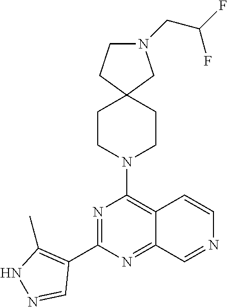 [SMILES: Cc1[nH]ncc1-c1nc(N2CCC3(CCN(CC(F)F)C3)CC2)c2ccncc2n1] | 4-(2-(2,2-difluoroethyl)-2,8- diazaspiro[4.5]decan-8-yl)-2-(5-methyl- 1H-pyrazol-4-yl)pyrido[3,4-d]pyrimidine |
| 221/ 222 | 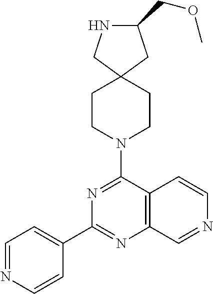 [SMILES: COC[C@H]1CC2(CCN(c3nc(-c4ccncc4)nc4cnccc34)CC2)CN1] | 4-(3-(methoxymethyl)-2,8- diazaspiro[4.5]decan-8-yl)-2-(pyridin-4- yl)pyrido[3,4-d]pyrimidine (R)-4-(3-(methoxymethyl)-2,8- diazaspiro[4.5]decan-8-yl)-2-(pyridin-4- yl)pyrido[3,4-d]pyrimidine (S)-4-(3-(methoxymethyl)-2,8- diazaspiro[4.5]decan-8-yl)-2-(pyridin-4- yl)pyrido[3,4-d]pyrimidine |
| | 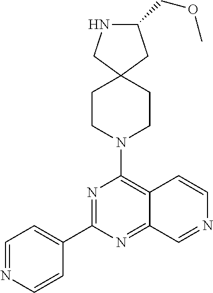 [SMILES: COC[C@@H]1CC2(CCN(c3nc(-c4ccncc4)nc4cnccc34)CC2)CN1] | |
| 223 | 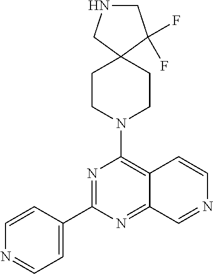 [SMILES: FC1(F)CNCC12CCN(c1nc(-c3ccncc3)nc3cnccc13)CC2] | 4-(4,4-difluoro-2,8-diazaspiro[4.5]decan- 8-yl)-2-(pyridin-4-yl)pyrido[3,4- d]pyrimidine |
| 224 | 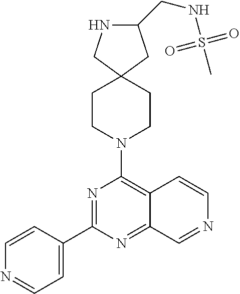 [SMILES: CS(=O)(=O)NCC1CC2(CCN(c3nc(-c4ccncc4)nc4cnccc34)CC2)CN1] | N-((8-(2-(pyridin-4-yl)pyrido[3,4- d]pyrimidin-4-yl)-2,8- diazaspiro[4.5]decan-3- yl)methyl)methanesulfonamide |
| 225 | 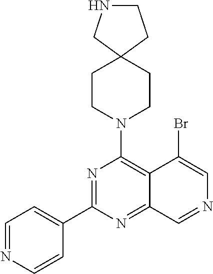 [SMILES: Brc1cncc2nc(-c3ccncc3)nc(N3CCC4(CCNC4)CC3)c12] | 5-bromo-2-(pyridin-4-yl)-4-(2,8- diazaspiro[4.5]decan-8-yl)pyrido[3,4- d]pyrimidine |
| 226 | 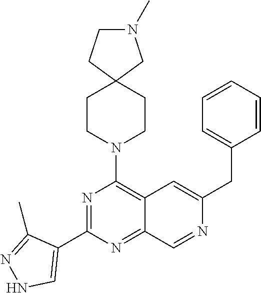 [SMILES: Cc1n[nH]cc1-c1nc(N2CCC3(CCN(C)C3)CC2)c2cc(Cc3ccccc3)ncc2n1] | 6-benzyl-2-(3-methyl-1H-pyrazol-4-yl)- 4-(2-methyl-2,8-diazaspiro[4.5]decan-8- yl)pyrido[3,4-d]pyrimidine |
| 227 | 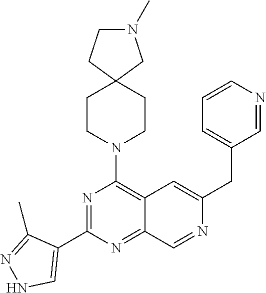 [SMILES: Cc1n[nH]cc1-c1nc(N2CCC3(CCN(C)C3)CC2)c2cc(Cc3cccnc3)ncc2n1] | 2-(3-methyl-1H-pyrazol-4-yl)-4-(2- methyl-2,8-diazaspiro[4.5]decan-8-yl)-6- (pyridin-3-ylmethyl)pyrido[3,4- d]pyrimidine |
| 228/ 229 | 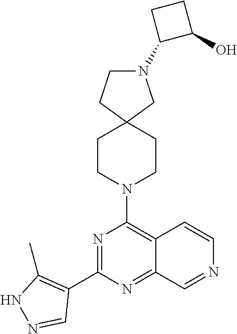 [SMILES: Cc1[nH]ncc1-c1nc(N2CCC3(CC2)CCN([C@@H]2CC[C@H]2O)C3)c2ccncc2n1] | (trans)-2-(8-(2-(5-methyl-1H-pyrazol-4- yl)pyrido[3,4-d]pyrimidin-4-yl)-2,8- diazaspiro[4.5]decan-2-yl)cyclobutan-1- ol (1R,2R)-2-(8-(2-(5-methyl-1H-pyrazol-4- yl)pyrido[3,4-d]pyrimidin-4-yl)-2,8- diazaspiro[4.5]decan-2-yl)cyclobutan-1- ol (1S,2S)-2-(8-(2-(5-methyl-1H-pyrazol-4- yl)pyrido[3,4-d]pyrimidin-4-yl)-2,8- diazaspiro[4.5]decan-2-yl)cyclobutan-1- ol |
| | 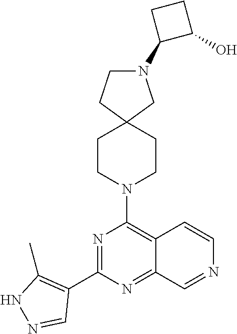 [SMILES: Cc1[nH]ncc1-c1nc(N2CCC3(CC2)CCN([C@H]2CC[C@@H]2O)C3)c2ccncc2n1] | |
| 230/ 231 | 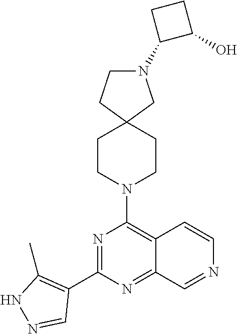 [SMILES: Cc1[nH]ncc1-c1nc(N2CCC3(CC2)CCN([C@@H]2CC[C@@H]2O)C3)c2ccncc2n1] | (cis)- 2-(8-(2-(5-methyl-1H-pyrazol-4- yl)pyrido[3,4-d]pyrimidin-4-yl)-2,8- diazaspiro[4.5]decan-2-yl)cyclobutan-1- ol (1S,2R)-2-(8-(2-(5-methyl-1H-pyrazol-4- yl)pyrido[3,4-d]pyrimidin-4-yl)-2,8- diazaspiro[4.5]decan-2-yl)cyclobutan-1- ol (1R,2S)-2-(8-(2-(5-methyl-1H-pyrazol-4- yl)pyrido[3,4-d]pyrimidin-4-yl)-2,8- diazaspiro[4.5]decan-2-yl)cyclobutan-1- ol |
| | 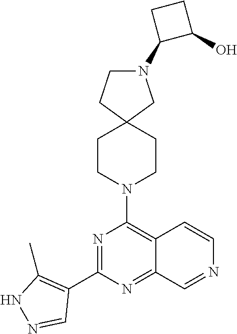 [SMILES: Cc1[nH]ncc1-c1nc(N2CCC3(CC2)CCN([C@H]2CC[C@H]2O)C3)c2ccncc2n1] | |
| 232 | 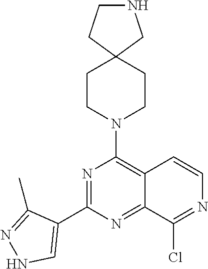 [SMILES: Cc1n[nH]cc1-c1nc(N2CCC3(CCNC3)CC2)c2ccnc(Cl)c2n1] | 8-chloro-2-(3-methyl-1H-pyrazol-4-yl)-4- (2,8-diazaspiro[4.5]decan-8- yl)pyrido[3,4-d]pyrimidine |
| 233 | 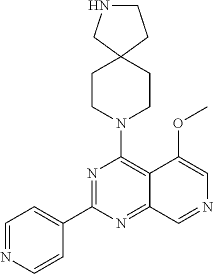 [SMILES: COc1cncc2nc(-c3ccncc3)nc(N3CCC4(CCNC4)CC3)c12] | 5-methoxy-2-(pyridin-4-yl)-4-(2,8- diazaspiro[4.5]decan-8-yl)pyrido[3,4- d]pyrimidine |
| 234/ 235 | 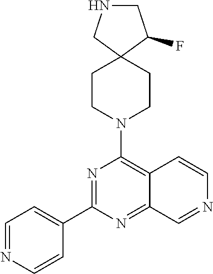 [SMILES: F[C@@H]1CNCC12CCN(c1nc(-c3ccncc3)nc3cnccc13)CC2] | 4-(4-fluoro-2,8-diazaspiro[4.5]decan-8- yl)-2-(pyridin-4-yl)pyrido[3,4- d]pyrimidine (S)-4-(4-fluoro-2,8-diazaspiro[4.5]decan- 8-yl)-2-(pyridin-4-yl)pyrido[3,4- d]pyrimidine (R)-4-(4-fluoro-2,8-diazaspiro[4.5]decan- 8-yl)-2-(pyridin-4-yl)pyrido[3,4- d]pyrimidine |
| | 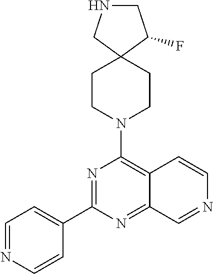 [SMILES: F[C@H]1CNCC12CCN(c1nc(-c3ccncc3)nc3cnccc13)CC2] | |
| 236 | 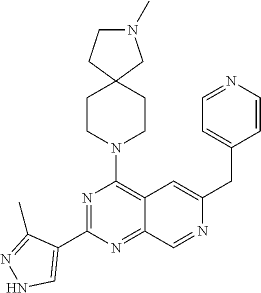 [SMILES: Cc1n[nH]cc1-c1nc(N2CCC3(CCN(C)C3)CC2)c2cc(Cc3ccncc3)ncc2n1] | 2-(3-methyl-1H-pyrazol-4-yl)-4-(2- methyl-2,8-diazaspiro[4.5]decan-8-yl)-6- (pyridin-4-ylmethyl)pyrido[3,4- d]pyrimidine |
| 237 | 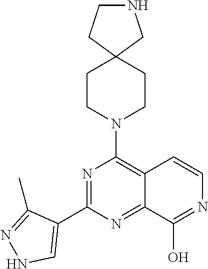 [SMILES: Cc1n[nH]cc1-c1nc(N2CCC3(CCNC3)CC2)c2ccnc(O)c2n1] | 2-(3-methyl-1H-pyrazol-4-yl)-4-(2,8- diazaspiro[4.5]decan-8-yl)pyrido[3,4- d]pyrimidin-8-ol |
| 238 | 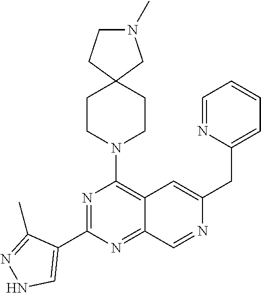 [SMILES: Cc1n[nH]cc1-c1nc(N2CCC3(CCN(C)C3)CC2)c2cc(Cc3ccccn3)ncc2n1] | 2-(3-methyl-1H-pyrazol-4-yl)-4-(2- methyl-2,8-diazaspiro[4.5]decan-8-yl)-6- (pyridin-2-ylmethyl)pyrido[3,4- d]pyrimidine |
| 239 | 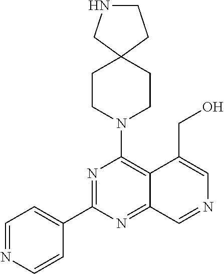 [SMILES: OCc1cncc2nc(-c3ccncc3)nc(N3CCC4(CCNC4)CC3)c12] | (2-(pyridin-4-yl)-4-(2,8- diazaspiro[4.5]decan-8-yl)pyrido[3,4- d]pyrimidin-5-yl)methanol |
| 240 | 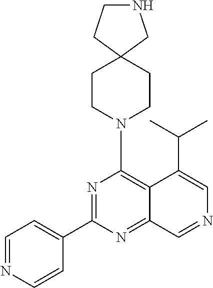 [SMILES: CC(C)c1cncc2nc(-c3ccncc3)nc(N3CCC4(CCNC4)CC3)c12] | 5-isopropyl-2-(pyridin-4-yl)-4-(2,8- diazaspiro[4.5]decan-8-yl)pyrido[3,4- d]pyrimidine |
| 241 | 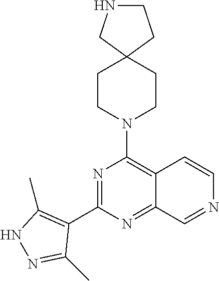 [SMILES: Cc1n[nH]c(C)c1-c1nc(N2CCC3(CCNC3)CC2)c2ccncc2n1] | 2-(3,5-dimethyl-1H-pyrazol-4-yl)-4-(2,8- diazaspiro[4.5]decan-8-yl)pyrido[3,4- d]pyrimidine |
| 242 | 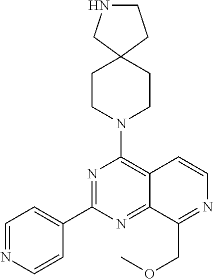 [SMILES: COCc1nccc2c(N3CCC4(CCNC4)CC3)nc(-c3ccncc3)nc12] | 8-(methoxymethyl)-2-(pyridin-4-yl)-4- (2,8-diazaspiro[4.5]decan-8- yl)pyrido[3,4-d]pyrimidine |
| 243 | 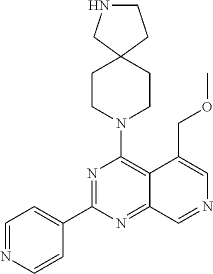 [SMILES: COCc1cncc2nc(-c3ccncc3)nc(N3CCC4(CCNC4)CC3)c12] | 5-(methoxymethyl)-2-(pyridin-4-yl)-4- (2,8-diazaspiro[4.5]decan-8- yl)pyrido[3,4-d]pyrimidine |
| 244 | 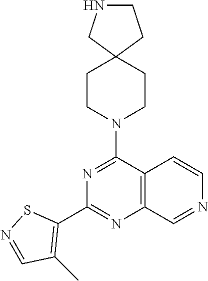 [SMILES: Cc1cnsc1-c1nc(N2CCC3(CCNC3)CC2)c2ccncc2n1] | 5-(4-(2,8-diazaspiro[4.5]decan-8- yl)pyrido[3,4-d]pyrimidin-2-yl)-4- methylisothiazole |
| 245 | 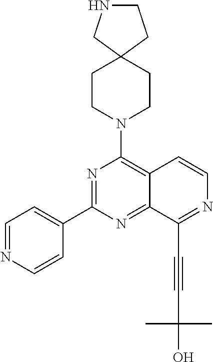 [SMILES: CC(C)(O)C#Cc1nccc2c(N3CCC4(CCNC4)CC3)nc(-c3ccncc3)nc12] | 2-methyl-4-(2-(pyridin-4-yl)-4-(2,8- diazaspiro[4.5]decan-8-yl)pyrido[3,4- d]pyrimidin-8-yl)but-3-yn-2-ol |
| 246 | 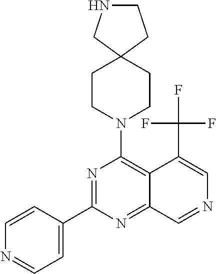 [SMILES: FC(F)(F)c1cncc2nc(-c3ccncc3)nc(N3CCC4(CCNC4)CC3)c12] | 2-(pyridin-4-yl)-4-(2,8- diazaspiro[4.5]decan-8-yl)-5- (trifluoromethyl)pyrido[3,4-d]pyrimidine |
| 247 | 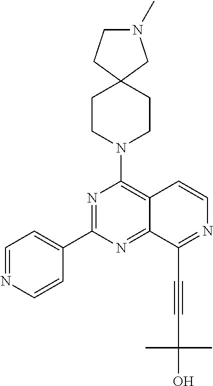 [SMILES: CN1CCC2(CCN(c3nc(-c4ccncc4)nc4c(C#CC(C)(C)O)nccc34)CC2)C1] | 2-methyl-4-(4-(2-methyl-2,8- diazaspiro[4.5]decan-8-yl)-2-(pyridin-4- yl)pyrido[3,4-d]pyrimidin-8-yl)but-3-yn- 2-ol |
| 248 | 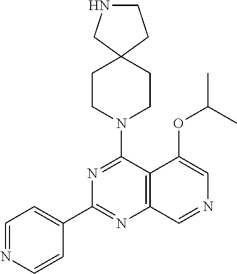 [SMILES: CC(C)Oc1cncc2nc(-c3ccncc3)nc(N3CCC4(CCNC4)CC3)c12] | 5-isopropoxy-2-(pyridin-4-yl)-4-(2,8- diazaspiro[4.5]decan-8-yl)pyrido[3,4- d]pyrimidine |
| 249 | 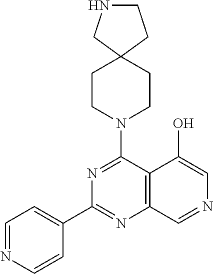 [SMILES: Oc1cncc2nc(-c3ccncc3)nc(N3CCC4(CCNC4)CC3)c12] | 2-(pyridin-4-yl)-4-(2,8- diazaspiro[4.5]decan-8-yl)pyrido[3,4- d]pyrimidin-5-ol |
| 250 | 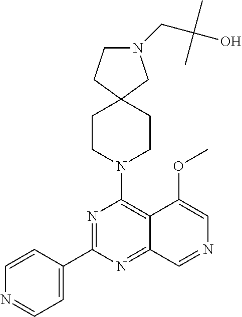 [SMILES: COc1cncc2nc(-c3ccncc3)nc(N3CCC4(CCN(CC(C)(C)O)C4)CC3)c12] | 1-(8-(5-methoxy-2-(pyridin-4- yl)pyrido[3,4-d]pyrimidin-4-yl)-2,8- diazaspiro[4.5]decan-2-yl)-2- methylpropan-2-ol |
| 251 | 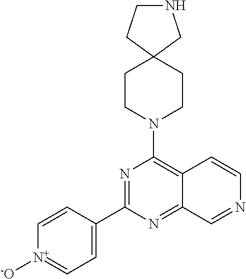 | 4-(4-(2,8-diazaspiro[4.5]decan-8- yl)pyrido[3,4-d]pyrimidin-2-yl)pyridine 1-oxide |
| 252 | 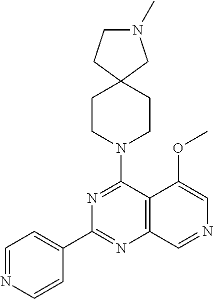 [SMILES: COc1cncc2nc(-c3ccncc3)nc(N3CCC4(CCN(C)C4)CC3)c12] | 5-methoxy-4-(2-methyl-2,8- diazaspiro[4.5]decan-8-yl)-2-(pyridin-4- yl)pyrido[3,4-d]pyrimidine |
| 253 | 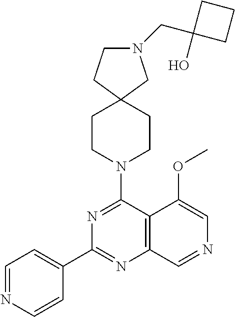 [SMILES: COc1cncc2nc(-c3ccncc3)nc(N3CCC4(CCN(CC5(O)CCC5)C4)CC3)c12] | 1-((8-(5-methoxy-2-(pyridin-4- yl)pyrido[3,4-d]pyrimidin-4-yl)-2,8- diazaspiro[4.5]decan-2- yl)methyl)cyclobutan-1-ol |
| 254 | 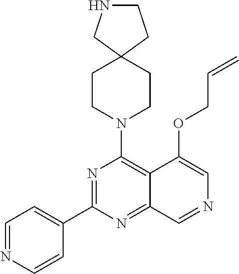 [SMILES: C=CCOc1cncc2nc(-c3ccncc3)nc(N3CCC4(CCNC4)CC3)c12] | 5-(allyloxy)-2-(pyridin-4-yl)-4-(2,8- diazaspiro[4.5]decan-8-yl)pyrido[3,4- d]pyrimidine |
| 255 | 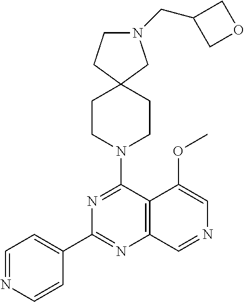 [SMILES: COc1cncc2nc(-c3ccncc3)nc(N3CCC4(CCN(CC5COC5)C4)CC3)c12] | 5-methoxy-4-(2-(oxetan-3-ylmethyl)-2,8- diazaspiro[4.5]decan-8-yl)-2-(pyridin-4- yl)pyrido[3,4-d]pyrimidine |
| 256 | 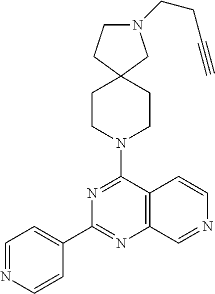 [SMILES: C#CCCN1CCC2(CCN(c3nc(-c4ccncc4)nc4cnccc34)CC2)C1] | 4-(2-(but-3-yn-1-yl)-2,8- diazaspiro[4.5]decan-8-yl)-2-(pyridin-4- yl)pyrido[3,4-d]pyrimidine |
| 257 | 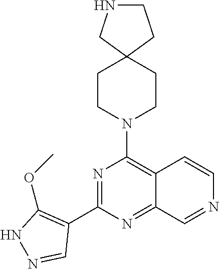 [SMILES: COc1[nH]ncc1-c1nc(N2CCC3(CCNC3)CC2)c2ccncc2n1] | 2-(5-methoxy-1H-pyrazol-4-yl)-4-(2,8- diazaspiro[4.5]decan-8-yl)pyrido[3,4- d]pyrimidine |
| 258 | 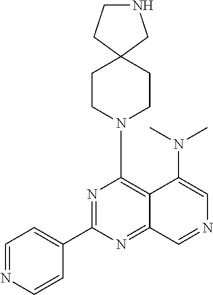 [SMILES: CN(C)c1cncc2nc(-c3ccncc3)nc(N3CCC4(CCNC4)CC3)c12] | N,N-dimethyl-2-(pyridin-4-yl)-4-(2,8- diazaspiro[4.5]decan-8-yl)pyrido[3,4- d]pyrimidin-5-amine |
| 259 | 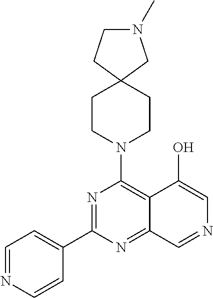 [SMILES: CN1CCC2(CCN(c3nc(-c4ccncc4)nc4cncc(O)c34)CC2)C1] | N,N-dimethyl-2-(pyridin-4-yl)-4-(2,8- diazaspiro[4.5]decan-8-yl)pyrido[3,4- d]pyrimidin-5-amine |
| 260 | 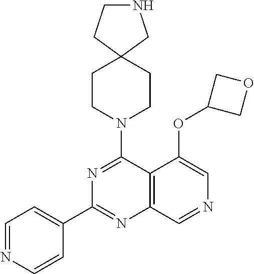 [SMILES: c1cc(-c2nc(N3CCC4(CCNC4)CC3)c3c(OC4COC4)cncc3n2)ccn1] | 5-(oxetan-3-yloxy)-2-(pyridin-4-yl)-4- (2,8-diazaspiro[4.5]decan-8- yl)pyrido[3,4-d]pyrimidine |
| 261 | 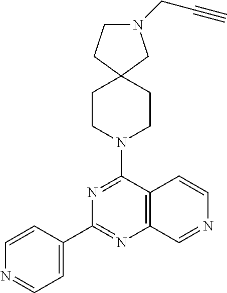 [SMILES: C#CCN1CCC2(CCN(c3nc(-c4ccncc4)nc4cnccc34)CC2)C1] | 4-(2-(prop-2-yn-1-yl)-2,8- diazaspiro[4.5]decan-8-yl)-2-(pyridin-4- yl)pyrido[3,4-d]pyrimidine |
| 262 | 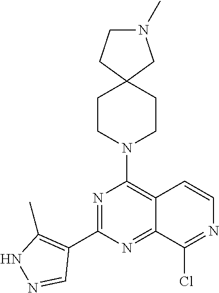 [SMILES: Cc1[nH]ncc1-c1nc(N2CCC3(CCN(C)C3)CC2)c2ccnc(Cl)c2n1] | 8-chloro-2-(5-methyl-1H-pyrazol-4-yl)-4- (2-methyl-2,8-diazaspiro[4.5]decan-8- yl)pyrido[3,4-d]pyrimidine |
| 263 | 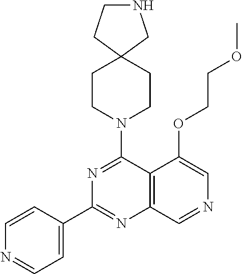 [SMILES: COCCOc1cncc2nc(-c3ccncc3)nc(N3CCC4(CCNC4)CC3)c12] | 5-(2-methoxyethoxy)-2-(pyridin-4-yl)-4- (2,8-diazaspiro[4.5]decan-8- yl)pyrido[3,4-d]pyrimidine |
| 264 | 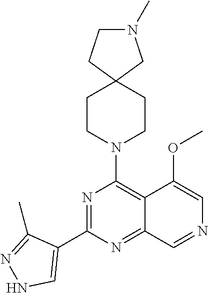 [SMILES: COc1cncc2nc(-c3c[nH]nc3C)nc(N3CCC4(CCN(C)C4)CC3)c12] | 5-methoxy-2-(3-methyl-1H-pyrazol-4- yl)-4-(2-methyl-2,8-diazaspiro[4.5]decan- 8-yl)pyrido[3,4-d]pyrimidine |
| 265 | 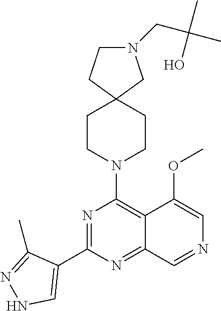 [SMILES: COc1cncc2nc(-c3c[nH]nc3C)nc(N3CCC4(CCN(CC(C)(C)O)C4)CC3)c12] | 1-(8-(5-methoxy-2-(3-methyl-1H- pyrazol-4-yl)pyrido[3,4-d]pyrimidin-4- yl)-2,8-diazaspiro[4.5]decan-2-yl)-2- methylpropan-2-ol |
| 266 | 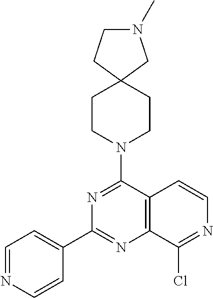 [SMILES: CN1CCC2(CCN(c3nc(-c4ccncc4)nc4c(Cl)nccc34)CC2)C1] | 8-chloro-4-(2-methyl-2,8- diazaspiro[4.5]decan-8-yl)-2-(pyridin-4- yl)pyrido[3,4-d]pyrimidine |
| 267 | 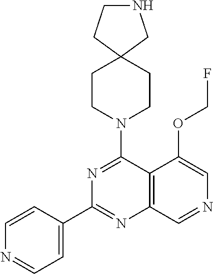 [SMILES: FCOc1cncc2nc(-c3ccncc3)nc(N3CCC4(CCNC4)CC3)c12] | 5-(fluoromethoxy)-2-(pyridin-4-yl)-4- (2,8-diazaspiro[4.5]decan-8- yl)pyrido[3,4-d]pyrimidine |
| 268 | 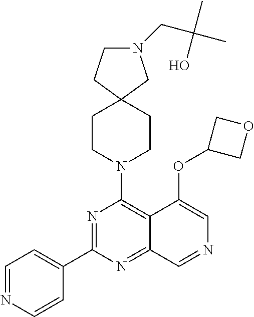 [SMILES: CC(C)(O)CN1CCC2(CCN(c3nc(-c4ccncc4)nc4cncc(OC5COC5)c34)CC2)C1] | 2-methyl-1-(8-(5-(oxetan-3-yloxy)-2- (pyridin-4-yl)pyrido[3,4-d]pyrimidin-4- yl)-2,8-diazaspiro[4.5]decan-2-yl)propan- 2-ol |
| 269 | 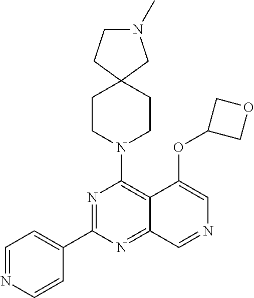 [SMILES: CN1CCC2(CCN(c3nc(-c4ccncc4)nc4cncc(OC5COC5)c34)CC2)C1] | 4-(2-methyl-2,8-diazaspiro[4.5]decan-8- yl)-5-(oxetan-3-yloxy)-2-(pyridin-4- yl)pyrido[3,4-d]pyrimidine |
| 270 | 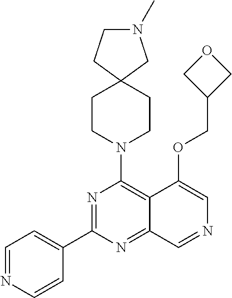 [SMILES: CN1CCC2(CCN(c3nc(-c4ccncc4)nc4cncc(OCC5COC5)c34)CC2)C1] | 4-(2-methyl-2,8-diazaspiro[4.5]decan-8- yl)-5-(oxetan-3-ylmethoxy)-2-(pyridin-4- yl)pyrido[3,4-d]pyrimidine |
| 271 | 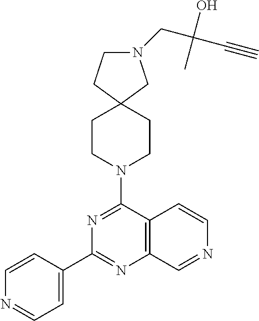 [SMILES: C#CC(C)(O)CN1CCC2(CCN(c3nc(-c4ccncc4)nc4cnccc34)CC2)C1] | 2-methyl-1-(8-(2-(pyridin-4- yl)pyrido[3,4-d]pyrimidin-4-yl)-2,8- diazaspiro[4.5]decan-2-yl)but-3-yn-2-ol |
| 272 | 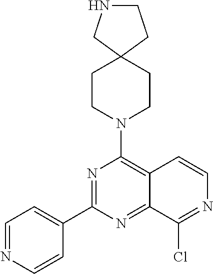 [SMILES: Clc1nccc2c(N3CCC4(CCNC4)CC3)nc(-c3ccncc3)nc12] | 8-chloro-2-(pyridin-4-yl)-4-(2,8- diazaspiro[4.5]decan-8-yl)pyrido[3,4- d]pyrimidine |
| 273 | 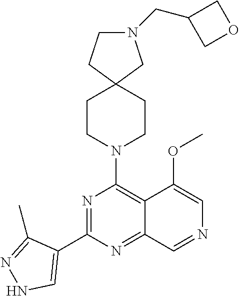 [SMILES: COc1cncc2nc(-c3c[nH]nc3C)nc(N3CCC4(CCN(CC5COC5)C4)CC3)c12] | 5-methoxy-2-(3-methyl-1H-pyrazol-4- yl)-4-(2-(oxetan-3-ylmethyl)-2,8- diazaspiro[4.5]decan-8-yl)pyrido[3,4- d]pyrimidine |
| 274 | 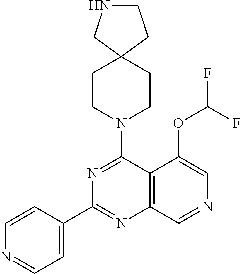 [SMILES: FC(F)Oc1cncc2nc(-c3ccncc3)nc(N3CCC4(CCNC4)CC3)c12] | 5-(difluoromethoxy)-2-(pyridin-4-yl)-4- (2,8-diazaspiro[4.5]decan-8- yl)pyrido[3,4-d]pyrimidine |
| 275 | 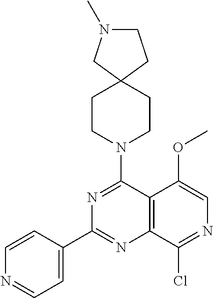 [SMILES: COc1cnc(Cl)c2nc(-c3ccncc3)nc(N3CCC4(CCN(C)C4)CC3)c12] | 8-chloro-5-methoxy-4-(2-methyl-2,8- diazaspiro[4.5]decan-8-yl)-2-(pyridin-4- yl)pyrido[3,4-d]pyrimidine |
| 276 | 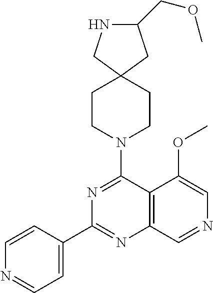 [SMILES: COCC1CC2(CCN(c3nc(-c4ccncc4)nc4cncc(OC)c34)CC2)CN1] | 5-methoxy-4-(3-(methoxymethyl)-2,8- diazaspiro[4.5]decan-8-yl)-2-(pyridin-4- yl)pyrido[3,4-d]pyrimidine |
| 277 | 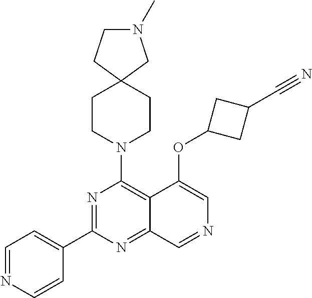 [SMILES: CN1CCC2(CCN(c3nc(-c4ccncc4)nc4cncc(OC5CC(C#N)C5)c34)CC2)C1] | 3-((4-(2-methyl-2,8-diazaspiro[4.5]decan- 8-yl)-2-(pyridin-4-yl)pyrido[3,4- d]pyrimidin-5-yl)oxy)cyclobutane-1- carbonitrile |
| 278 | 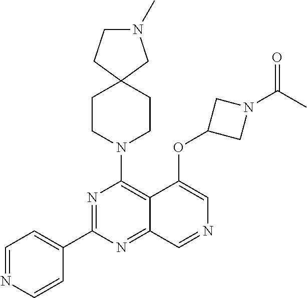 [SMILES: CC(=O)N1CC(Oc2cncc3nc(-c4ccncc4)nc(N4CCC5(CCN(C)C5)CC4)c23)C1] | 1-(3-((4-(2-methyl-2,8- diazaspiro[4.5]decan-8-yl)-2-(pyridin-4- yl)pyrido[3,4-d]pyrimidin-5- yl)oxy)azetidin-1-yl)ethan-1-one |
| 279 | 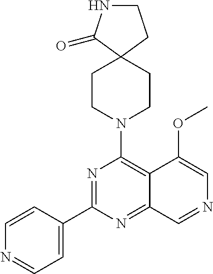 [SMILES: COc1cncc2nc(-c3ccncc3)nc(N3CCC4(CCNC4=O)CC3)c12] | 8-(5-methoxy-2-(pyridin-4-yl)pyrido[3,4- d]pyrimidin-4-yl)-2,8- diazaspiro[4.5]decan-1-one |
| 280 | 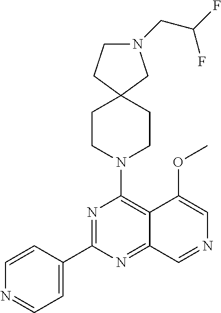 [SMILES: COc1cncc2nc(-c3ccncc3)nc(N3CCC4(CCN(CC(F)F)C4)CC3)c12] | 4-(2-(2,2-difluoroethyl)-2,8- diazaspiro[4.5]decan-8-yl)-5-methoxy-2- (pyridin-4-yl)pyrido[3,4-d]pyrimidine |
| 281 | 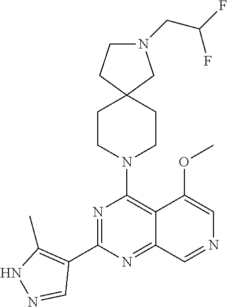 [SMILES: COc1cncc2nc(-c3cn[nH]c3C)nc(N3CCC4(CCN(CC(F)F)C4)CC3)c12] | 4-(2-(2,2-difluoroethyl)-2,8- diazaspiro[4.5]decan-8-yl)-5-methoxy-2- (5-methyl-1H-pyrazol-4-yl)pyrido[3,4- d]pyrimidine |
| 282 | 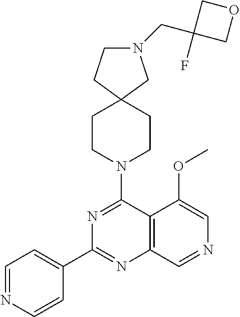 [SMILES: COc1cncc2nc(-c3ccncc3)nc(N3CCC4(CCN(CC5(F)COC5)C4)CC3)c12] | 4-(2-((3-fluorooxetan-3-yl)methyl)-2,8- diazaspiro[4.5]decan-8-yl)-5-methoxy-2- (pyridin-4-yl)pyrido[3,4-d]pyrimidine |
| 283 | 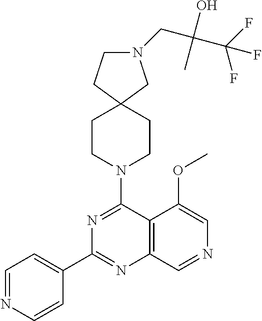 [SMILES: COc1cncc2nc(-c3ccncc3)nc(N3CCC4(CCN(CC(C)(O)C(F)(F)F)C4)CC3)c12] | 1,1,1-trifluoro-3-(8-(5-methoxy-2- (pyridin-4-yl)pyrido[3,4-d]pyrimidin-4- yl)-2,8-diazaspiro[4.5]decan-2-yl)-2- methylpropan-2-ol |
| 284 | 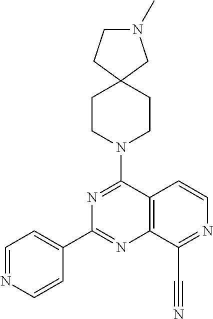 [SMILES: CN1CCC2(CCN(c3nc(-c4ccncc4)nc4c(C#N)nccc34)CC2)C1] | 4-(2-methyl-2,8-diazaspiro[4.5]decan-8- yl)-2-(pyridin-4-yl)pyrido[3,4- d]pyrimidine-8-carbonitrile |
| 285 | 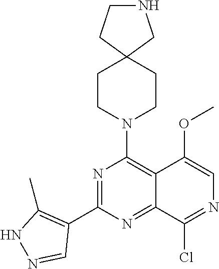 [SMILES: COc1cnc(Cl)c2nc(-c3cn[nH]c3C)nc(N3CCC4(CCNC4)CC3)c12] | 8-chloro-5-methoxy-2-(5-methyl-1H- pyrazol-4-yl)-4-(2,8- diazaspiro[4.5]decan-8-yl)pyrido[3,4- d]pyrimidine |
| 286 | 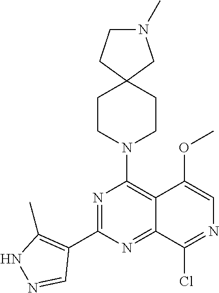 [SMILES: COc1cnc(Cl)c2nc(-c3cn[nH]c3C)nc(N3CCC4(CCN(C)C4)CC3)c12] | 8-chloro-5-methoxy-2-(5-methyl-1H- pyrazol-4-yl)-4-(2-methyl-2,8- diazaspiro[4.5]decan-8-yl)pyrido[3,4- d]pyrimidine |
| 287 | 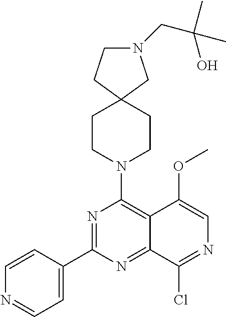 [SMILES: COc1cnc(Cl)c2nc(-c3ccncc3)nc(N3CCC4(CCN(CC(C)(C)O)C4)CC3)c12] | 1-(8-(8-chloro-5-methoxy-2-(pyridin-4- yl)pyrido[3,4-d]pyrimidin-4-yl)-2,8- diazaspiro[4.5]decan-2-yl)-2- methylpropan-2-ol |
| 288 | 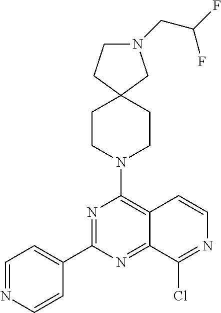 [SMILES: FC(F)CN1CCC2(CCN(c3nc(-c4ccncc4)nc4c(Cl)nccc34)CC2)C1] | 8-chloro-4-(2-(2,2-difluoroethyl)-2,8- diazaspiro[4.5]decan-8-yl)-2-(pyridin-4- yl)pyrido[3,4-d]pyrimidine |
| 289 | 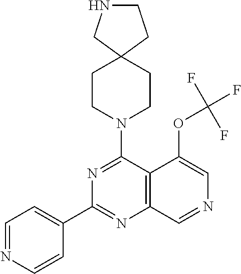 [SMILES: FC(F)(F)Oc1cncc2nc(-c3ccncc3)nc(N3CCC4(CCNC4)CC3)c12] | 2-(pyridin-4-yl)-4-(2,8- diazaspiro[4.5]decan-8-yl)-5- (trifluoromethoxy)pyrido[3,4- d]pyrimidine |
| 290 | 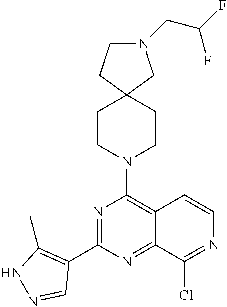 [SMILES: Cc1[nH]ncc1-c1nc(N2CCC3(CCN(CC(F)F)C3)CC2)c2ccnc(Cl)c2n1] | 8-chloro-4-(2-(2,2-difluoroethyl)-2,8- diazaspiro[4.5]decan-8-yl)-2-(5-methyl- 1H-pyrazol-4-yl)pyrido[3,4-d]pyrimidine |
| 291 | 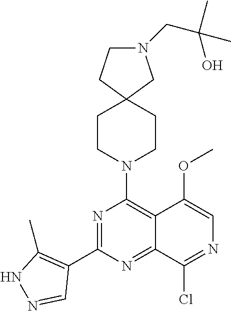 [SMILES: COc1cnc(Cl)c2nc(-c3cn[nH]c3C)nc(N3CCC4(CCN(CC(C)(C)O)C4)CC3)c12] | 1-(8-(8-chloro-5-methoxy-2-(5-methyl- 1H-pyrazol-4-yl)pyrido[3,4-d]pyrimidin- 4-yl)-2,8-diazaspiro[4.5]decan-2-yl)-2- methylpropan-2-ol |
| 292 | 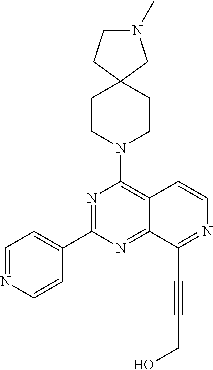 [SMILES: CN1CCC2(CCN(c3nc(-c4ccncc4)nc4c(C#CCO)nccc34)CC2)C1] | 3-(4-(2-methyl-2,8-diazaspiro[4.5]decan- 8-yl)-2-(pyridin-4-yl)pyrido[3,4- d]pyrimidin-8-yl)prop-2-yn-1-ol |

In some embodiments, provided is a compound selected from Compound Nos. 101-292 in Table 1, or a salt (e.g., a pharmaceutically acceptable salt) thereof. In some embodiments, the compound is selected from Compound Nos. 101-201 in Table 1, or a salt (e.g., a pharmaceutically acceptable salt) thereof. In some embodiments, the compound is selected from Compound Nos. 101-198 in Table 1, or a salt (e.g., a pharmaceutically acceptable salt) thereof. In some embodiments, the compound is selected from Compound Nos. 202-292 in Table 1, or a salt (e.g., a pharmaceutically acceptable salt) thereof.

Compounds of Formula (I) described herein or a salt thereof may exist in stereoisomeric forms (e.g., it contains one or more asymmetric carbon atoms). The individual stereoisomers (enantiomers and diastereomers) and mixtures of these are included within the scope of the subject matter disclosed herein. It is to be understood that the subject matter disclosed herein includes combinations and subsets of the particular groups described herein. The scope of the subject matter disclosed herein includes mixtures of stereoisomers as well as purified enantiomers or enantiomerically/diastereomerically enriched mixtures. It is to be understood that the subject matter disclosed herein includes combinations and subsets of the particular groups defined herein.

It is also understood that a compound or salt of Formulas (I) may exist in tautomeric forms other than that shown in the formula and these are also included within the scope of the subject matter disclosed herein. For example, a pyrazolyl group may exist as either or both tautomers shown below:

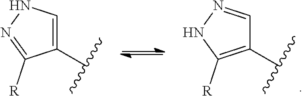

When one of the particular tautomer is dipicted in a structure drawing, both tautomers are intended regardless whether the one dipicted is the major or the minor tautomer in existence.

The subject matter disclosed herein also includes isotopically-labelled forms of the compounds described herein, but for the fact that one or more atoms are replaced by an atom having an atomic mass or mass number different from the atomic mass or mass number usually found in nature. Examples of isotopes that can be incorporated into compounds described herein and pharmaceutically acceptable salts thereof include isotopes of hydrogen, carbon, nitrogen, oxygen, phosphorous, sulphur, fluorine, iodine, and chlorine, such as ²H, ³H, ¹¹C, ¹³C, ¹⁴C, ¹⁵N, 17O, ¹⁸O, ³¹P, ³²P, ³⁵S, ¹⁸F, ³⁶C, ¹²³I and ¹²⁵I.

The subject matter disclosed herein includes prodrugs, metabolites, derivatives, and pharmaceutically acceptable salts of compounds of Formula (I). Metabolites of the compounds of Formula (I) include compounds produced by a process comprising contacting a compound of Formula (I) with a mammal for a period of time sufficient to yield a metabolic product thereof.

If the compound of Formula (I) is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound of Formula (I) is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

A compound of Formula (I) can be in the form of a “prodrug,” which includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Prodrugs which are converted to active forms through other mechanisms in vivo are also included. In aspects, the compounds of the invention are prodrugs of any of the formulae herein.

### General Synthetic Method

Compounds of the present disclosure can be made by a variety of methods depicted in the illustrative synthetic reaction schemes shown and described below, where R groups are as described for Formula (I). The starting materials and reagents used in preparing these compounds generally are either available from commercial suppliers, such as Sigma-Aldrich Chemical Co., or are prepared by methods known to those skilled in the art following procedures set forth in references such as Fieser and Fieser's Reagents for Organic Synthesis; Wiley & Sons: New York, vol. 1-21; R. C. LaRock, Comprehensive Organic Transformations, 2nd edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds.) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, vol. 1-40; and subsequent editions. The following synthetic reaction schemes are merely illustrative of some methods by which the compounds of the present disclosure can be synthesized, and various modifications to these synthetic reaction schemes can be made and will be suggested to one skilled in the art having referred to the disclosure contained herein.

For illustrative purposes, the reaction Schemes below provide routes for synthesizing the compounds of the invention as well as key intermediates. For a more detailed description of the individual reaction steps, see the Examples section below. Those skilled in the art will appreciate that other synthetic routes may be used. Although some specific starting materials and reagents are depicted in the Schemes and discussed below, other starting materials and reagents can be substituted to provide a variety of derivatives or reaction conditions. In addition, many of the compounds prepared by the methods described below can be further modified in light of this disclosure using conventional chemistry well known to those skilled in the art.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, or, about 20° C.

Some compounds in following schemes are depicted with generalized substituents; however, one skilled in the art will immediately appreciate that the nature of the substituents can varied to afford the various compounds contemplated in this invention. Moreover, the reaction conditions are exemplary and alternative conditions are well known. The reaction sequences in the following examples are not meant to limit the scope of the invention as set forth in the claims.

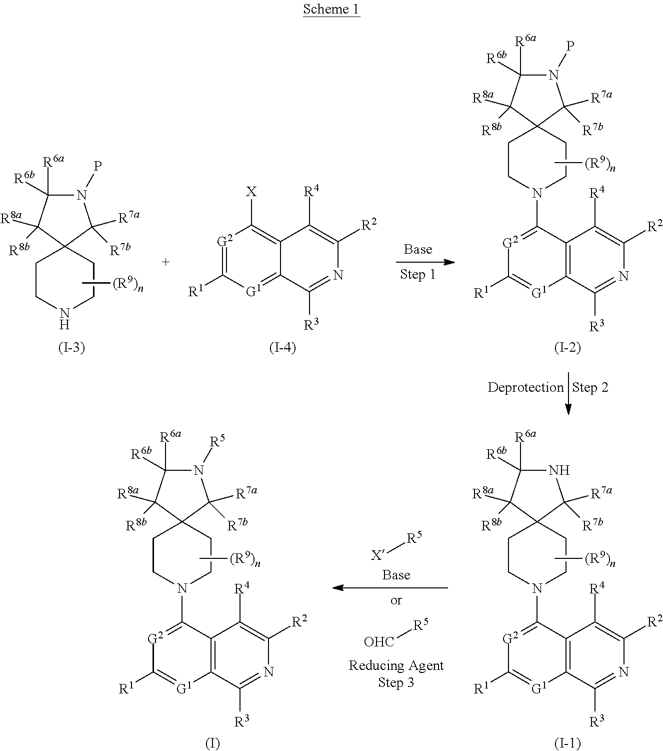

Scheme 1 shows a general synthetic scheme for preparing a compound of Formula (I), wherein R¹, R², R³, R⁴, G¹, G², R⁵, R^{6a}, R^{6b}, R^{7a}, R^{7b}, R^{8a}, R^{8b}, R⁹, and n are as detailed herein, via an SnAr reaction of a heteroaromatic compound of Formula (I-4) and a 2-N-protected 2,8-diazaspiro[4.5]decane compound of Formula (1-3), wherein X is selected from the group consisting of Cl, Br, I, F, OMs, and OTs, P may be any suitable protecting group known to those skilled in the art, including, but not limited to, Boc, Fmoc, Cbz, and the like, and X′ is a leaving group including, but not limited to, Cl, Br, I, OMs, and OTs. In Step 1, a compound of Formula (I-4) is reacted with a compound of Formula (1-3), in the presence of any suitable organic or inorganic base to form a comound of Formula (1-2). In Step 2, the protecting group P is removed from the compound of Formula (1-2) to form a compound of Formula (I-1). Suitable deprotection techniques are known in the art and will vary depending on the protecting group used. In one embodiment, the protecting group P is Boc, and the compound of Formula (I-2) is deprotected by contacting the compound of Formula (I-2) with a strong or weak acid, such as TFA, TsOH, HCl, or the like. In Step 3, the compound of Formula (I-1) is contacted with a compound of the formula R⁵—X′, where X′ is a leaving group, in the presence of a suitable inorganic or organic base, or is contacted with an aldehyde compound of the formula R⁵—CHO in the presence of a reducing agent to form the compound of Formula (I). Suitable reducing agents include, but are not limited to, NaBH₄, NaBH₃CN, NaBH(OAc)₃, and the like.

The compound of (1-4) can be made by methods detailed herein (including the demonstrative examples) and methods known in the art from appropriate starting materials and reagents. The method may vary depending on the nature of R¹, R², R³, R⁴, G¹ and G².

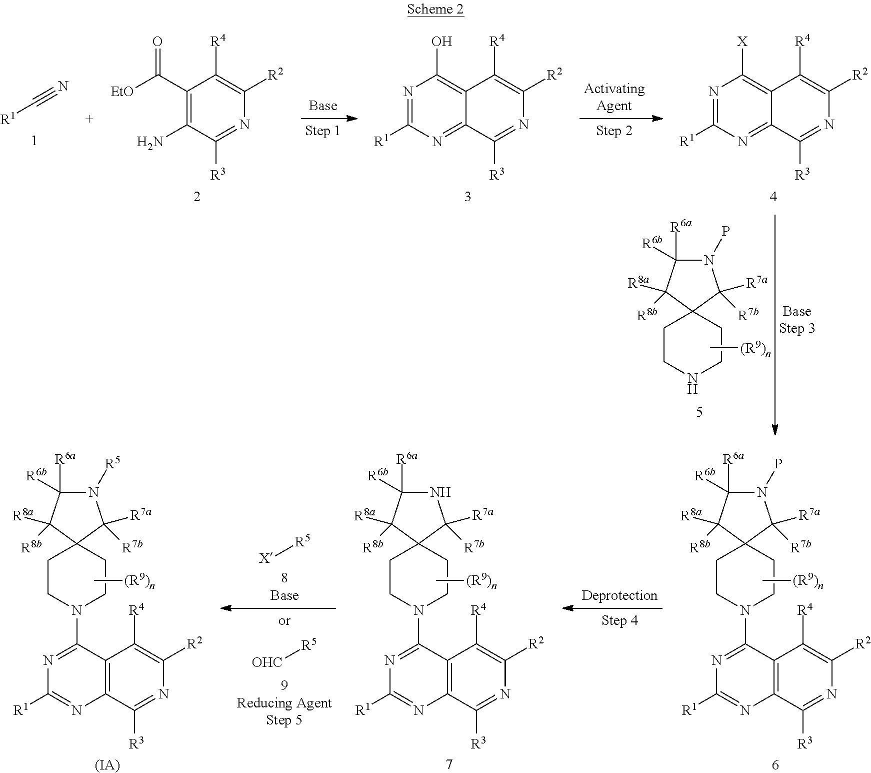

Scheme 2 shows a general synthetic scheme for preparing a compound of Formula (IA), wherein R¹, R², R³, R⁴, R⁵, R^{6a}, R^{6b}, R^{7a}, R^{7b}, R^{8a}, R^{8b}, R⁹, and n are as detailed herein, via an SnAr reaction of a pyrido[3,4-d]pyrimidine compound of Formula 4 and a 2-N-protected 2,8-diazaspiro[4.5]decane compound of Formula 5, wherein X is selected from the group consisting of Cl, Br, I, F, OMs, and OTs, P may be any suitable protecting group known to those skilled in the art, including, but not limited to, Boc, Fmoc, Cbz, and the like, and X′ is a leaving group including, but not limited to, Cl, Br, I, OMs, and OTs. In Step 1, a compound of Formula 1 and a compound of Formula 2 are mixed in the presence of any suitable organic or inorganic base to form a compound of Formula 3. In Step 2, the compound of Formula 3 is contacted with an activating agent to form a compound of Formula 4. Suitable activating agents include, but are not limited to, POCl₃, POBr₃, MsCl, TsCl, and the like. In Step 3, the compound of Formula 4 is reacted with a compound of Formula 5, in the presence of any suitable organic or inorganic base to form a compound of Formula 6. In Step 4, the protecting group P is removed from the compound of Formula 6 to form a compound of Formula 7. Suitable deprotection techniques are known in the art and will vary depending on the protecting group used. In one embodiment, the protecting group P is Boc, and the compound of Formula 6 is deprotected by contacting the compound of Formula 6 with a strong or weak acid, such as TFA, TsOH, HCl, or the like. In Step 5, the compound of Formula 7 is contacted with a compound of Formula 8 in the presence of a suitable inorganic or organic base, or is contacted with a compound of Formula 9 in the presence of a reducing agent to form the compound of Formula (I). Suitable reducing agents include, but are not limited to, NaBH₄, NaBH₃CN, NaBH(OAc)₃, and the like.

Alternatively, the compound of Formula 3 can be made by reacting an imidamide of Formula 1a with a 3-fluoronicotinic acid of Formula 2a in the presence of a base, or by reacting an aldehyde of Formula 1b with a 3-aminonicotinamide of Formula 2b in the presence of an oxidant (e.g., copper oxide).

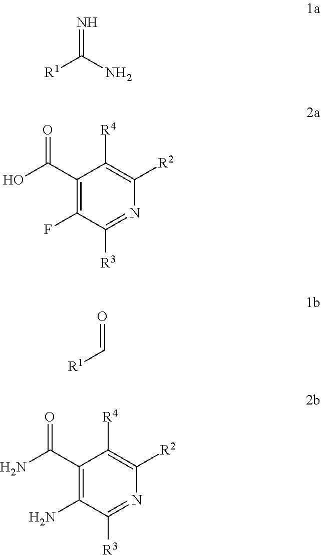

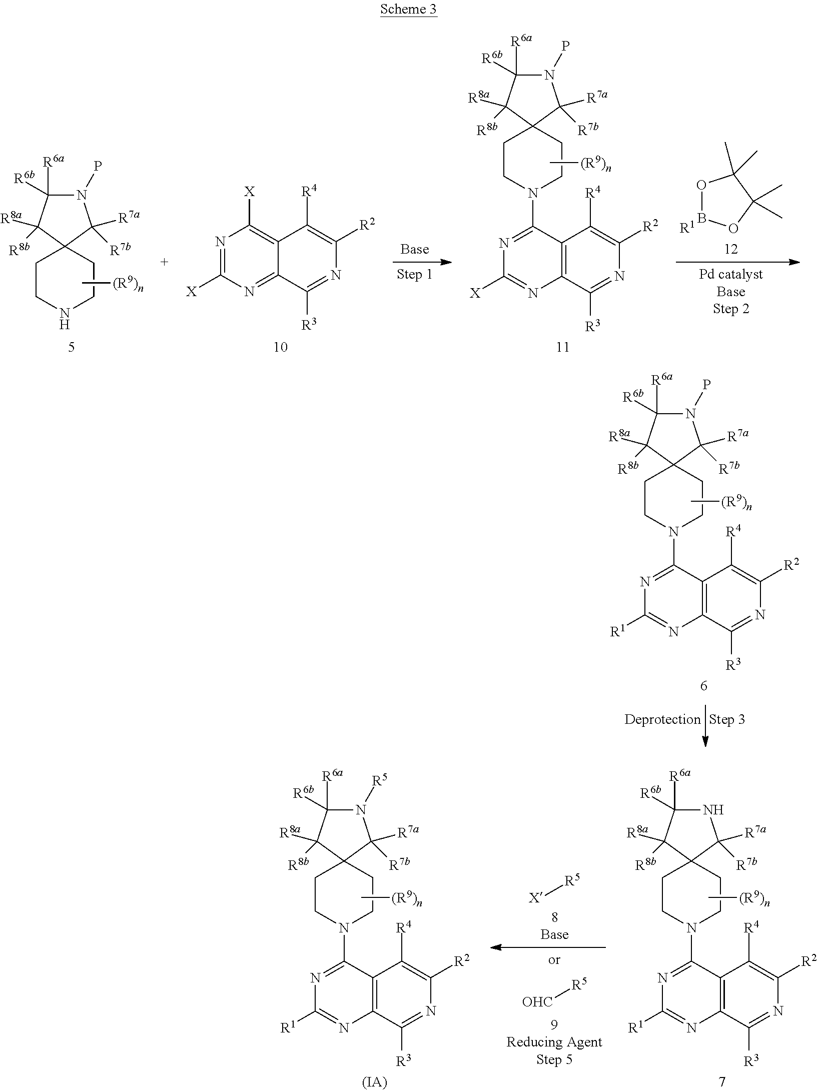

Scheme 3 shows a general synthetic scheme for preparing a compound of Formula (IA), wherein R¹, R², R³, R⁴, R⁵, R^{6a}, R^{6b}, R^{7a}, R^{7b}, R^{8a}, R^{8b}, R⁹, and n are as detailed herein, from cross coupling of a heteroaryl boronate of Formula 12 with a N-protected (pyrido[3,4-d]pyrimidin-4-yl)-2,8-diazaspiro[4.5]decane compound of Formula 11, which in turn can be prepared via an SnAr reaction of pyrido[3,4-d]pyrimidine compound of Formula 10 and a 2-N-protected 2,8-diazaspiro[4.5]decane compound of Formula 5, wherein X is selected from the group consisting of Cl, Br, I, F, OMs, and OTs, P may be any suitable protecting group known to those skilled in the art, including, but not limited to, Boc, Fmoc, Cbz, and the like, and X′ is a leaving group including, but not limited to, Cl, Br, I, OMs, and OTs. In Step 1, a compound of Formula 5 is coupled to a compound of Formula 10 in the presence of any suitable inorganic or organic base to form a compound of Formula 11. In Step 2, the compound of Formula 11 is contacted with a compound of Formula 12 in the presence of a Pd catalyst and any suitable organic or inorganic base to form a compound of Formula 6. Any suitable Pd catalyst may be used including, but not limited to, Pd(PPh₃)₄. In Step 3, protecting group P is removed from the compound of Formula 6 to form a compound of Formula 7. Suitable deprotection techniques are known in the art and will vary depending on the protecting group used. In one embodiment, the protecting group P is Boc, and the compound of Formula 6 is deprotected by contacting the compound of Formula 6 with a strong or weak acid, such as TFA, TsOH, HCl, or the like. In Step 4, the compound of Formula 7 is contacted with a compound of Formula 8 in the presence of a suitable inorganic or organic base, or with a compound of Formula 9 in the presence of a reducing agent to form the compound of Formula (IA). Suitable reducing agents include, but are not limited to, NaBH₄, NaBH₃CN, NaBH(OAc)₃, and the like. In step 2, a suitable R¹—Zn or R¹—Sn compound may be used as an alternative to the boronate of Formula 12, with a suitable Pd catalyst and base.

In the methods of making a compound of Formula (I) or (IA) as exemplified in the reaction sequences in Schemes 1-3, the compound of Formula (I) or (IA) may be made using starting materials having substitutents that differ from the corresponding substituents in the intermediates and the final products. The substituents in the starting materials may be a precursor which is converted to the desirable substituent in the next intermediate or the final product. For example, a starting material having an R⁴ group which is fluoro is converted in the subsequent steps to an intermediate or final product having an R⁴ group which is an alkoxy (e.g., methoxy). In other examples, a final product having an R⁴ group which is ethyl (—CH₂CH₃) is made from a starting material having an R⁴ group which is vinyl (—CH═CH₂), or a final product having an R⁴ group which is hydroxymethyl (—CH₂OH) is made by reducing an intermediate having an R⁴ group which is formyl (—CH═O), which is made from a starting material having an R⁴ group which is vinyl (—CH═CH₂). Likewise, a final product having an R³ group which is alkynyl (e.g., —C≡CC(Me)₂OH) is made via Stille coupling using a starting material having an R³ group which is chloro. A compound of formula (I) or (IA) may also be made from another compound of formula (I) or (IA) by modifying one or more of the substituents. For example, a compound of formulate (IA) having an R² group that is a 1-hydroxybenzyl or 1-pyridyl-1-hydroxymethyl can be made from a compound of formulate (IA) having an R² group that is a benzyl or pyridylmethyl respectively.

Thus, in one aspect, provided is a method for making a compound of Formula (I):

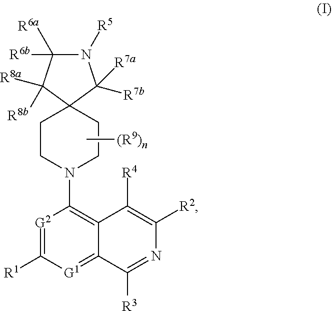

or a salt (e.g., a pharmaceutically acceptable salt), solvate (e.g., hydrate), prodrug, metabolites or derivative thereof, wherein R¹, R², R³, R⁴, G¹, G², R⁵, R^{6a}, R^{6b}, R^{7a}, R^{7b}, R^{8a}, R^{8b}, R⁹, and n are as defined herein, the method comprising:
a) reacting a compound of Formula (I-4):

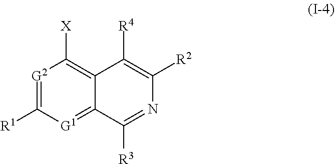

with a compound of Formula (I-3):

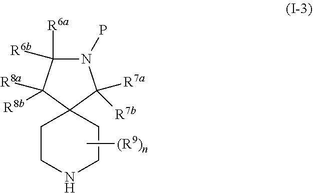

in the presence of a base to produce a compound of Formula (I-2):

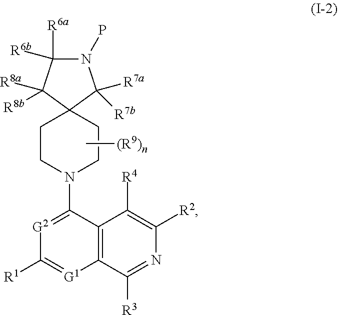

wherein X is selected from the group consisting of Cl, Br, I, F, OMs, and OTs; and P is a protecting group;
b) removing protecting group P from the compound of Formula (I-2) to produce a compound of Formula (I-1):

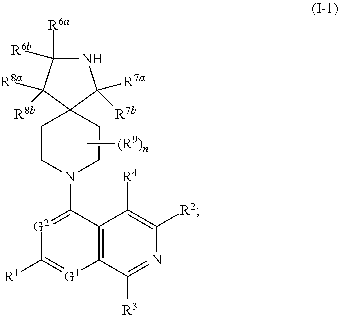

c) converting the compound of Formula (I-1) to the compound of Formula (I).

In one embodiment, the compound of Formula (I-1) is converted to the compound of Formula (I) by contacting the compound of Formula (I-1) with a compound of Formula R⁵—X′, wherein X′ is a leaving group, in the presence of a base. In some embodiments, X′ is selected from the group consisting of Cl, Br, I, OMs, OTs.

In another embodiment, the compound of Formula (I-1) is converted to the compound of Formula (I) by contacting the compound of Formula (I-1) with an aldehyde compound of Formula R⁵—CHO, in the presence of a reducing agent. In one embodiment, the reducing agent is selected from the group consisting of NaBH₄, NaBH₃CN, and NaBH(OAc)₃.

In one aspect, provided is a method for making a compound of Formula (IA):

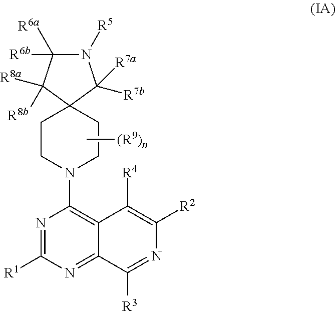

or a salt (e.g., a pharmaceutically acceptable salt), solvate (e.g., hydrate), prodrug, metabolites or derivative thereof, wherein R¹, R², R³, R⁴, R^{6a}, R^{6b}, R^{7a}, R^{7b}, R^{8a}, R^{8b}, R⁹, and n are as defined herein, the method comprising:
a) reacting a compound of Formula 4:

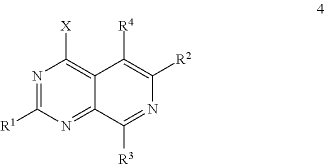

with a compound of Formula 5:

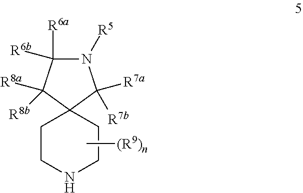

in the presence of a base to produce a compound of Formula 6:

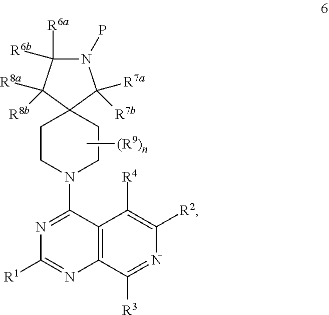

wherein X is selected from the group consisting of Cl, Br, I, F, OMs, and OTs; and P is a protecting group;
b) removing protecting group P from the compound of Formula 6 to produce a compound of Formula 7:

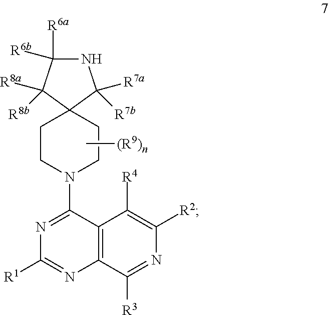

and
c) converting the compound of Formula 7 to the compound of Formula (IA).

In one embodiment, the compound of Formula 7 is converted to the compound of Formula (IA) by contacting the compound of Formula 7 with a compound of Formula 8 in the presence of a base:

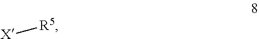

wherein X′ is a leaving group. In some embodiments, X′ is selected from the group consisting of Cl, Br, I, OMs, OTs. In some embodiments, the compound of Formula 8 is an epoxide wherein X′ is an oxide which becomes a hydroxyl group that is part of the resulting R⁵ group.

In another embodiment, the compound of Formula 7 is converted to the compound of Formula (IA) by contacting the compound of Formula 7 with a compound of Formula 9 in the presence of a reducing agent:

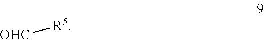

In one embodiment, the reducing agent is selected from the group consisting of NaBH₄, NaBH₃CN, and NaBH(OAc)₃.

In another embodiment, the method further comprises producing the compound of Formula 4. In particular, the method may further comprise:
d) mixing a compound of Formula 1

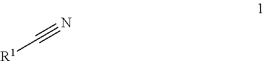

with a compound of Formula 2:

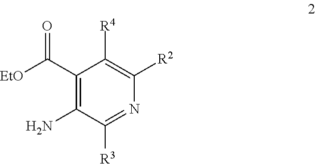

in the presence of a base to form a compound of Formula 3:

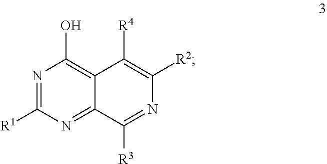

and
e) contacting the compound of Formula 3 with an activating agent to form the compound of Formula 4.

In some embodiments, the method further comprises producing the compound of Formula 4, comprising a step of:
d) mixing a compound of Formula 1a

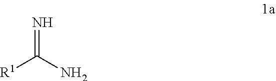

with a compound of Formula 2a:

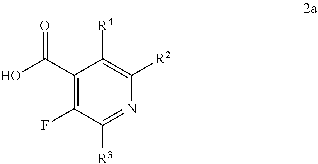

in the presence of a base to form a compound of Formula 3.

In some embodiments, the method further comprises producing the compound of Formula 4, comprising a step of:
d) mixing a compound of Formula 1b

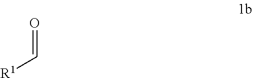

with a compound of Formula 2b:

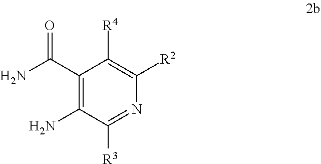

in the presence of an oxidant (e.g., CuO) to form a compound of Formula 3.

In one aspect, the activing agent is selected from the group consisting of POCl₃, POBr₃, MsCl, and TsCl.

In another aspect, provided is a method for making a compound of Formula (IA):

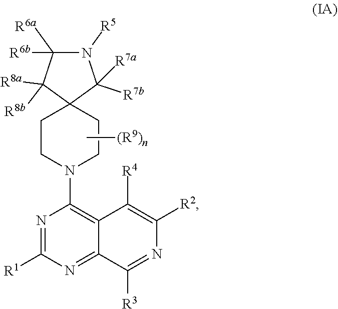

or a salt (e.g., a pharmaceutically acceptable salt), solvate (e.g., hydrate), prodrug, metabolites or derivative thereof, wherein R¹, R², R³, R⁴, R⁵, R^{6a}, R^{6b}, R^{7a}, R^{7b}, R^{8a}, R^{8b}, R⁹, and n are as defined herein, the method comprising:
a) contacting a compound of Formula 12:

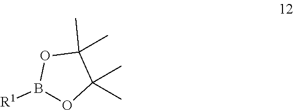

with a compound of Formula 11:

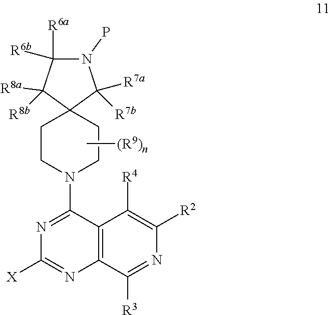

in the presence of a palladium catalyst and a base to form a compound of Formula 6:

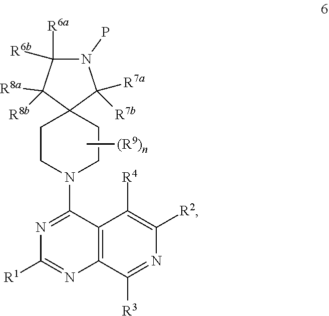

wherein X is selected from the group consisting of Cl, Br, I, F, OMs, and OTs, and P is a protecting group;
b) removing protecting group P from the compound of Formula 6 to produce a compound of Formula 7:

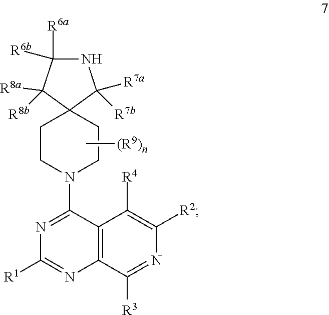

and
c) converting the compound of Formula 7 to the compound of Formula (IA).

In one embodiment, the compound of Formula 7 is converted to the compound of Formula (IA) by contacting the compound of Formula 7 with a compound of Formula 8 in the presence of a base:

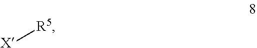

wherein X′ is a leaving group. In some embodiments, X′ is selected from the group consisting of Cl, Br, I, OMs, OTs.

In another embodiment, the compound of Formula 7 is converted to the compound of Formula (IA) by contacting the compound of Formula 7 with a compound of Formula 9 in the presence of a reducing agent:

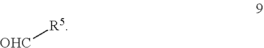

In one embodiment, the reducing agent is selected from the group consisting of NaBH₄, NaBH₃CN, and NaBH(OAc)₃.

In another aspect, the method further comprises producing the compound of Formula 11. In particular, the method further comprises:
d) coupling a compound of Formula 5:

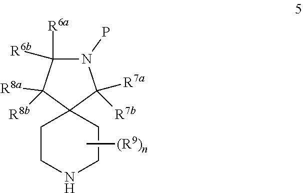

to a compound of Formula 10:

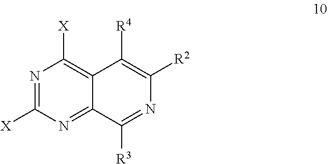

in the presence of a base to form the compound of Formula 11, wherein X and P are as defined herein.

Also provided is a product made according to any one or more of the methods or processes described herein.

### Pharmaceutical Compositions and Formulations

The presently disclosed compounds can be formulated into pharmaceutical compositions along with a pharmaceutically acceptable carrier or excipient.

Compounds of Formula (I), or variations thereof, can be formulated in accordance with standard pharmaceutical practice as a pharmaceutical composition. According to this aspect, there is provided a pharmaceutical composition comprising a compound of Formula (I), or variations thereof such as Formulae (IA), (IB) and (IC), in association with a pharmaceutically acceptable excipient, diluent or carrier. The preferred composition depends on the method of administration, and typically comprises one or more conventional pharmaceutically acceptable carriers, adjuvants, and/or vehicles (together referred to as “excipients”). Such compositions can be formulated for various routes of systemic or local delivery for example, by oral administration, topical administration, transmucosal administration, rectal administration, intravaginal administration, or administration by subcutaneous, intrathecal, intravenous, intramuscular, intraperitoneal, intranasal, intraocular or intraventricular injection.

Solid dosage forms for oral administration include, for example, capsules, tablets, pills, powders, and granules. In such compositions, the compounds or salts are ordinarily combined with one or more excipients. If administered per os, the compounds or salts can be mixed with, for example, lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation, as can be provided in, for example, a dispersion of the compound or salt in hydroxypropylmethyl cellulose. In the case of capsules, tablets, and pills, the dosage forms also can comprise pH modifiers, such as sodium citrate; magnesium or calcium carbonate or bicarbonate; tartaric acid, fumaric acid, citric acid, succinic acid, malic acid, and phosphoric acid and combinations thereof. Tablets and pills additionally can be prepared with enteric coatings.

Liquid dosage forms for oral administration include, for example, pharmaceutically acceptable emulsions (including both oil-in-water and water-in-oil emulsions), solutions (including both aqueous and non-aqueous solutions), suspensions (including both aqueous and non-aqueous suspensions), syrups, and elixirs containing inert diluents commonly used in the art (e.g., water). Such compositions also can comprise, for example, wetting, emulsifying, suspending, sweeting and flavoring agents.

Parenteral administration includes subcutaneous injections, intravenous injections, intramuscular injections, intrastemal injections, and infusion. Injectable preparations (e.g., sterile injectable aqueous or oleaginous suspensions) can be formulated according to the known art using suitable dispersing, wetting agents, and/or suspending agents. Acceptable vehicles and solvents include, for example, water, 1,3-butanediol, Ringer's solution, isotonic sodium chloride solution, bland fixed oils (e.g., synthetic mono- or diglycerides), fatty acids (e.g., oleic acid), dimethyl acetamide, surfactants (e.g., ionic and non-ionic detergents), and/or polyethylene glycols.

Formulations for parenteral administration may, for example, be prepared from sterile powders or granules having one or more of the excipients mentioned for use in the formulations for oral administration. A compound or salt of the invention can be dissolved in water, polyethylene glycol, propylene glycol, ethanol, corn oil, cottonseed oil, peanut oil, sesame oil, benzyl alcohol, sodium chloride, and/or various pH modifiers. The pH may be adjusted, if necessary, with a suitable acid, base, or pH modifier.

Suppositories for rectal administration can be prepared by, for example, mixing a compound or salt of the invention with a suitable nonirritating excipient that is solid at ordinary temperatures, but liquid at the rectal temperature, and will therefore melt in the rectum to release the drug. Suitable excipients include, for example, cocoa butter; synthetic mono-, di-, or triglycerides, fatty acids, and/or polyethylene glycols.

Compounds of the present disclosure can be formulated for administration topically to the skin or mucosa, i.e., dermally or transdermally. Such administration can include the use, e.g., of transdermal patches or iontophoresis devices.

Besides those representative dosage forms described above, pharmaceutically acceptable excipients and carriers are generally known to those skilled in the art and are thus included in the instant invention. Formulation of drugs is generally discussed in, for example, Hoover, J., Remington's Pharmaceutical Sciences (Mack Publishing Co., 1975) and Ansel's Pharmaceutical Dosage Forms and Drug Delivery Systems (Lippincott Williams & Wilkins, 2005), and subsequent editions.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical compositions comprising a compound of Formula (I) or variations thereof such as Formulae (IA), (IB) and (IC), can be formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the compound to be administered will be governed by such considerations, and is the minimum amount necessary to prevent, ameliorate, or treat the coagulation factor mediated disorder. In some embodiments, the amount is below the amount that is toxic to the host or renders the host more susceptible to bleeding.

### Methods of Use

The presently disclosed compounds find use in inhibiting the activity of LATS1/2.

In an embodiment, the subject matter disclosed herein is directed to a method of inhibiting LATS1/2 in a cell, the method comprising contacting the cell with an effective amount of a compound of Formula (I), or variations thereof such as Formulae (IA), (IB) and (IC), or a pharmaceutically acceptable salt thereof, or a pharmaceutical composition described herein.

In another embodiment, the subject matter disclosed herein is directed to a method for treating a disease or condition, the method comprising administering to a subject in need thereof an effective amount of a compound of Formula (I), or variations thereof such as Formulae (IA), (IB) and (IC), or a pharmaceutically acceptable salt thereof, or a pharmaceutical composition described herein. In certain aspects of this embodiment, the disease or condition is mediated by LATS1/2. In some aspects, the disease or condition is acute respiratory distress syndrome (ARDS). In other aspects, the disease or condition is idiopathic pulmonary fibrosis (IPF).

In another aspect, provided is a method for promoting tissue regeneration after an injury or a method of treating a disease or condition that can benefit from LATS1/2 inhibition, the method comprising administering to a subject in need thereof an effective amount of a compound of Formula (I), or variations thereof, such as Formulae (IA), (IB) and (IC), or a pharmaceutically acceptable salt thereof. In one embodiment, the disease or condition is ARDS. In other aspects, the disease or condition is IPF.

Also provided herein is a compound of Formula (I), or variations thereof, such as Formulae (IA), (IB) and (IC), or a pharmaceutically acceptable salt thereof, for use in a method of inhibiting LATS1/2 in a cell.

Also provided herein is a compound of Formula (I), or variations thereof, such as Formulae (IA), (IB) and (IC), or a pharmaceutically acceptable salt thereof, for use in a method of promoting tissue regeneration after injury or in a method for treating a disease or condition that can benefit from LATS1/2 inhibition. In one embodiment, the disease or condition is ARDS. In other aspects, the disease or condition is IPF.

In another aspect, provided is the use of a compound of Formula (I), or variations thereof, such as Formulae (IA), (IB) and (IC), or a pharmaceutically acceptable salt thereof, in a method detailed herein (e.g., promoting tissue regeneration after injury, or treatment of ARDS or IPF).

Also provided is use of a compound of Formula (I), or any variation thereof, such as Formulae (IA), (IB) and (IC), or a pharmaceutically acceptable salt thereof, for the manufacture of a medicament for use in a method detailed herein (e.g., promoting tissue regeneration after injury or treatment of ARDS or IPF).

In any of the embodiments described herein, the subject may be a human.

Further provided are kits for carrying out the methods detailed herein, which comprises one or more compounds described herein or a pharmaceutical composition comprising a compound described herein. The kits may employ any of the compounds disclosed herein. In one variation, the kit employs a compound described herein or a pharmaceutically acceptable salt thereof. The kits may be used for any one or more of the uses described herein, and, accordingly, may contain instructions for use, e.g., for use in promoting tissue regeneration after injury and/or in the treatment of diseases or conditions that can benefit from LATS1/2 inhibition. In some embodiments, the kit contains instructions for use in the treatment of ARDS. In some embodiments, the kit contains instructions for use in the treatment of IPF.

Kits generally comprise suitable packaging. The kits may comprise one or more containers comprising any compound or composition described herein. Each component (if there is more than one component) can be packaged in separate containers or some components can be combined in one container where cross-reactivity and shelf life permit. One or more components of a kit may be sterile and/or may be contained within sterile packaging.

The kits may be in unit dosage forms, bulk packages (e.g., multi-dose packages) or sub-unit doses. For example, kits may be provided that contain sufficient dosages of a compound as disclosed herein (e.g., a therapeutically effective amount) and/or a second pharmaceutically active compound useful for a LATS1/2-dependent disorder (e.g., ARDS) to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to a subject.

The following examples are offered by way of illustration and not by way of limitation.

### Additional Embodiments

Embodiment 1. A compound of Formula (I):

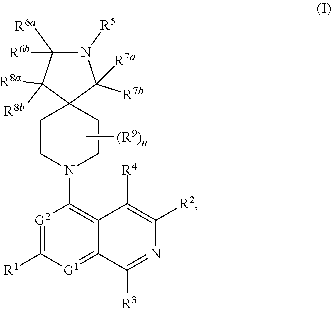

or a pharmaceutically acceptable salt thereof, wherein:
R¹ is 5- to 14-membered heteroaryl optionally substituted with 1, 2, 3, 4 or 5 substituents independently selected from R¹⁰;
R² is hydrogen, C₁₋₆ alkyl or —O(C₁₋₆ alkyl), wherein each C₁₋₆ alkyl is optionally substituted with 1, 2, 3, 4 or 5 substituents independently selected from R¹⁰;
R³ is hydrogen, C₁₋₆ alkyl or —O(C₁₋₆ alkyl), wherein each C₁₋₆ alkyl is optionally substituted with 1, 2, 3, 4 or 5 substituents independently selected from R¹⁰;
R⁴ is hydrogen, halogen, cyano, —O(C₁₋₆ alkyl), C₁₋₆ alkyl or C₃₋₆ cycloalkyl, wherein the C₁₋₆ alkyl and C₃₋₆ cycloalkyl are each optionally substituted with 1, 2, 3, 4 or 5 substituents independently selected from R¹⁰;
G¹ is N or CR⁴¹, G² is N or CR⁴², provided that one or both of G¹ and G² are N;
R⁴¹ and R⁴² are independently hydrogen, halogen, cyano, —O(C₁₋₆ alkyl), C₁₋₆ alkyl or C₃₋₆ cycloalkyl, wherein the C₁₋₆ alkyl and C₃₋₆ cycloalkyl are each optionally substituted with 1, 2, 3, 4 or 5 substituents independently selected from R¹⁰;
R⁵ is hydrogen, C₁₋₆ alkyl, C₃₋₈ cycloalkyl, C₆₋₁₄ aryl, 5- to 14-membered heteroaryl, 3- to 14-membered heterocyclyl, —C(O)R¹⁴, —C(O)OR¹⁵ or —C(O)NR^{16a}R^{16b}, wherein the C₁₋₆ alkyl, C₃₋₈ cycloalkyl, C₆₋₁₄ aryl, 5- to 14-membered heteroaryl and 3- to 14-membered heterocyclyl of R⁵ are each optionally substituted with 1, 2, 3, 4 or 5 substituents independently selected from R¹⁰;
each R^{6a} and R^{6b} is independently hydrogen, C₁₋₆ alkyl, C₃₋₈ cycloalkyl, C₆₋₁₀ aryl, 5- to 14-membered heteroaryl, 3- to 12-membered heterocyclyl, —C(O)R¹⁴, —C(O)OR¹⁵ or —C(O)NR^{16a}R^{16b}, wherein the C₁₋₆ alkyl, C₃₋₈ cycloalkyl, C₆₋₁₀ aryl, 5- to 14-membered heteroaryl, and 3- to 12-membered heterocyclyl of R^{6a} and R^{6b} are each optionally substituted with 1, 2, 3, 4 or 5 substituents independently selected from R¹⁰; or R^{6a} and R^{6b} are taken together with the carbon to which they are attached to form a carbonyl;
each R^{7a} and R^{7b} is independently hydrogen or C₁₋₆ alkyl optionally substituted with 1, 2, 3, 4 or 5 substituents independently selected from R¹⁰; or R^{7a} and R^{7b} are taken together with the carbon to which they are attached to form a carbonyl;
each R^{8a} and R^{8b} is independently hydrogen, halogen, hydroxyl, —O(C₁₋₆ alkyl) or C₁₋₆ alkyl, wherein each C₁₋₆ alkyl is optionally substituted with 1, 2, 3, 4 or 5 substituents independently selected from R¹⁰;
n is 0 to 8;
each R⁹ is independently C₁₋₆ alkyl; or two geminal R^{g} groups, if present, are taken together with the carbon to which they are attached to form a carbonyl;
each R¹⁴ is independently hydrogen or C₁₋₆ alkyl;
each R¹⁵ is independently C₁₋₆ alkyl;
each R^{16a} and R^{16b} is independently hydrogen or C₁₋₆ alkyl; or R^{16a} and R^{16b} are taken together with the nitrogen atom to which they are attached to form a 4- to 12-membered heterocyclyl optionally substituted with 1, 2, 3, 4 or 5 substituents independently selected from R¹⁰;
each R¹⁰ is independently oxo, C₁₋₆ alkyl, C₂₋₆ alkenyl, C₂₋₆ alkynyl, C₃₋₈ cycloalkyl, C₆₋₁₄ aryl, 5- to 14-membered heteroaryl, 3- to 14-membered heterocyclyl, halogen, cyano, —C(O)R^{a}, —C(O)OR^{b}, —C(O)NR^{c}R^{d}, —OR^{b}, —OC(O)R^{a}, —OC(O)NR^{c}R^{d}, —SR^{b}, —S(O)R^{c}, —S(O)₂R^{c}, —S(O)(═NH)R^{c}, —S(O)₂NR^{c}R^{d}, —NR^{c}R^{d}, —N(R¹)C(O)R^{a}, —N(R¹)C(O)OR^{b}, —N(R)C(O)NR^{c}R^{d}, —N(R^{f})S(O)₂R^{c}, —N(R¹)S(O)₂NR^{c}R^{d} or —P(O)R^{g}R^{h}, wherein the C₁₋₆ alkyl, C₂₋₆ alkenyl, C₂₋₆ alkynyl, C₃₋₈ cycloalkyl, C₆₋₁₄ aryl, 5- to 14-membered heteroaryl and 3- to 14-membered heterocyclyl of R¹⁰ are each optionally substituted with 1, 2, 3 or 4 substituents independently selected from R¹¹;
each R^{a} is independently hydrogen, C₁₋₆ alkyl, C₂₋₆ alkenyl, C₂₋₆ alkynyl, C₃₋₈ cycloalkyl, C₆₋₁₀ aryl, 5- to 10-membered heteroaryl or 3- to 12-membered heterocyclyl, wherein the C₁₋₆ alkyl, C₂₋₆ alkenyl, C₂₋₆ alkynyl, C₃₋₈ cycloalkyl, C₆₋₁₀ aryl, 5- to 10-membered heteroaryl and 3- to 12-membered heterocyclyl of R^{a} are each optionally substituted with 1, 2, 3 or 4 substituents independently selected from R¹¹;
each R^{b} is independently hydrogen, C₁₋₆ alkyl, C₃₋₈ cycloalkyl, C₆₋₁₀ aryl, 5- to 10-membered heteroaryl or 3- to 12-membered heterocyclyl, wherein the C₁₋₆ alkyl, C₃₋₈ cycloalkyl, C₆₋₁₀ aryl, 5- to 10-membered heteroaryl and 3- to 12-membered heterocyclyl of R^{b} are each optionally substituted with 1, 2, 3 or 4 substituents independently selected from R¹¹;
each R^{c} and R^{d} is independently hydrogen, C₁₋₆ alkyl, C₃₋₈ cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{c} and R^{d} are each optionally substituted with 1, 2, 3 or 4 substituents independently selected from R¹¹;
   or R^{c} and R^{d} are taken together with the nitrogen atom to which they are attached to form a 4- to 12-membered heterocyclyl optionally substituted with 1, 2, 3 or 4 substituents independently selected from R¹¹;
each R^{c} is independently C₁₋₆ alkyl, C₃₋₈ cycloalkyl, C₆₋₁₀ aryl, 5- to 10-membered heteroaryl or 3- to 12-membered heterocyclyl, wherein the C₁₋₆ alkyl, C₃₋₈ cycloalkyl, C₆₋₁₀ aryl, 5- to 10-membered heteroaryl and 3- to 12-membered heterocyclyl of R^{c} are each optionally substituted with 1, 2, 3 or 4 substituents independently selected from R¹¹;
each R^{f2} is independently hydrogen or C₁₋₆ alkyl;
each R^{g} and R^{h} is independently C₁₋₆ alkyl, C₃₋₈ cycloalkyl, C₆₋₁₀ aryl, 5- to 10-membered heteroaryl, 3- to 12-membered heterocyclyl or —O—C₁₋₆ alkyl; wherein the C₁₋₆ alkyl, C₃₋₈ cycloalkyl, C₆₋₁₀ aryl, 5- to 10-membered heteroaryl and 3- to 12-membered heterocyclyl of R^{g} and R^{h} are each optionally substituted with 1, 2, 3 or 4 substituents independently selected from R¹¹;
   or R^{g} and R^{h} are taken together with the phosphorus atom to which they are attached to form a 4- to 12-membered heterocyclyl optionally substituted with 1, 2, 3 or 4 substituents independently selected from R¹¹;
each R¹¹ is independently oxo, C₁₋₆ alkyl, C₂₋₆ alkenyl, C₂₋₆ alkynyl, C₃₋₆ cycloalkyl, C₆₋₁₀ aryl, 5- to 10-membered heteroaryl, 3- to 8-membered heterocyclyl, halogen, cyano, —C(O)R^{a1}, —C(O)OR^{b1}, —C(O)NR^{c1}R^{d1}, —OR^{b1}, —OC(O)R^{a1}, —OC(O)NR^{c1}R^{d1}, —SR^{b1}, —S(O)R^{c1}, —S(O)₂R^{c1}, —S(O)₂NR^{c1}R^{d1}, —NR^{c1}R^{d1}, —N(R^{f1})C(O)R^{a1}, —N(R^{f1})C(O)OR^{b1}, —N(R^{f1})C(O)NR^{c1}R^{d1}, —N(R^{f1})S(O)₂R^{c1}, —N(R^{f1})S(O)₂NR^{c1}R^{d1} or —P(O)R^{g1}R^{h1}; wherein the C₁₋₆ alkyl, C₂₋₆ alkenyl, C₂₋₆ alkynyl, C₃₋₆ cycloalkyl, C₆₋₁₀ aryl, 5- to 10-membered heteroaryl and 3- to 8-membered heterocyclyl of R¹¹ are each optionally substituted with 1, 2, 3 or 4 substituents independently selected from R¹²;
each R^{a1} is independently hydrogen, C₁₋₆ alkyl, C₂₋₆ alkenyl, C₂₋₆ alkynyl, C₃₋₆ cycloalkyl, C₆₋₁₀ aryl, 5- to 10-membered heteroaryl or 3- to 8-membered heterocyclyl; wherein the C₁₋₆ alkyl, C₂₋₆ alkenyl, C₂₋₆ alkynyl, C₃₋₆ cycloalkyl, C₆₋₁₀ aryl, 5- to 10-membered heteroaryl and 3- to 8-membered heterocyclyl of R^{a1} are each optionally substituted with 1, 2, 3 or 4 substituents independently selected from R¹²;
each R^{b1} is independently hydrogen, C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{b1} are each optionally substituted with 1, 2, 3 or 4 substituents independently selected from R¹²;
each R^{c1} and R^{d1} is independently hydrogen, C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{c1} and R^{d1} are each optionally substituted with 1, 2, 3 or 4 substituents independently selected from R¹²;
   or R^{c1} and R^{d1} are taken together with the nitrogen atom to which they are attached to form a 4- to 8-membered heterocyclyl optionally substituted with 1, 2, 3 or 4 substituents independently selected from R¹²;
each R^{c1} is independently C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{c1} are each optionally substituted with 1, 2, 3 or 4 substituents independently selected from R¹²;
each R^{f1} is independently hydrogen or C₁₋₆ alkyl;
each R^{g1} and R^{h1} is independently C₁₋₆ alkyl, C₃₋₆ cycloalkyl, C₆₋₁₀ aryl, 5- to 10-membered heteroaryl, 3- to 8-membered heterocyclyl, or —O—C₁₋₆ alkyl; wherein the C₁₋₆ alkyl, C₃₋₆ cycloalkyl, C₆₋₁₀ aryl, 5- to 10-membered heteroaryl and 3- to 8-membered heterocyclyl of R^{g1} and R^{h1} are each optionally substituted with 1, 2, 3 or 4 substituents independently selected from R¹²;
   or R^{g1} and R^{h1} are taken together with the phosphorus atom to which they are attached to form a 4- to 8-membered heterocyclyl optionally substituted with 1, 2, 3 or 4 substituents independently selected from R¹²;
each R¹² is independently oxo, C₁₋₆ alkyl, C₃₋₆ cycloalkyl, C₆ aryl, 5- to 6-membered heteroaryl, 3- to 6-membered heterocyclyl, halogen, cyano, —C(O)R^{a2}, —C(O)OR^{b2}, —C(O)NR^{c2}R^{d2}, —OR^{b2}, —OC(O)R^{a2}, —OC(O)NR^{c2}R^{d2}, —S(O)₂R^{c2}, —S(O)₂NR^{c2}R^{d2}, —NR^{c2}R^{d2}, —N(R^{f})C(O)R^{a2}, —N(R^{f})C(O)OR^{b2}, —N(R²)C(O)NR^{c2}R^{d2}, —N(R^{f})S(O)₂R^{c2}, —N(R^{f2})S(O)₂NR^{c2}R^{d2} or —P(O)R^{g2}R^{h2}; wherein the C₁₋₆ alkyl, C₃₋₆ cycloalkyl, C₆ aryl, 5- to 6-membered heteroaryl and 3- to 6-membered heterocyclyl of R¹² are each optionally substituted with 1, 2, 3 or 4 substituents independently selected from R¹³;
each R^{a2} is independently hydrogen, C₁₋₆ alkyl, C₃₋₆ cycloalkyl, C₆ aryl, 5- to 6-membered heteroaryl or 3- to 6-membered heterocyclyl; wherein the C₁₋₆ alkyl, C₃₋₆ cycloalkyl, C₆ aryl, 5- to 6-membered heteroaryl and 3- to 6-membered heterocyclyl of R^{a2} are each optionally substituted with 1, 2, 3 or 4 substituents independently selected from R¹³;
each R^{b2} is independently hydrogen, C₁₋₆ alkyl, C₃₋₆ cycloalkyl or 3- to 6-membered heterocyclyl; wherein the C₁₋₆ alkyl, C₃₋₆ cycloalkyl and 3- to 6-membered heterocyclyl of R^{b2} are each optionally substituted with 1, 2, 3 or 4 substituents independently selected from R¹³;
each R^{c2} and R^{d2} is independently hydrogen, C₁₋₆ alkyl, C₃₋₆ cycloalkyl or 3- to 8-membered heterocyclyl; wherein the C₁₋₆ alkyl, C₃₋₆ cycloalkyl and 3- to 8-membered heterocyclyl of R^{c2} and R^{d2} are each optionally substituted with 1, 2, 3 or 4 substituents independently selected from R¹³;
   or R^{c2} and R^{d2} are taken together with the nitrogen atom to which they are attached to form a 4- to 6-membered heterocyclyl optionally substituted with 1, 2, 3 or 4 substituents independently selected from R¹³;
each R^{c2} is independently C₁₋₆ alkyl, C₃₋₆ cycloalkyl, C₆ aryl, 5- to 6-membered heteroaryl or 3- to 6-membered heterocyclyl; wherein the C₁₋₆ alkyl, C₃₋₆ cycloalkyl, C₆ aryl, 5 to 6-membered heteroaryl and 3- to 6-membered heterocyclyl of R^{c2} are each optionally substituted with 1, 2, 3 or 4 substituents independently selected from R¹³;
each RV is independently hydrogen or C₁₋₆ alkyl;
each R^{g2} and R^{h2} is independently C₁₋₆ alkyl, C₃₋₆ cycloalkyl, 3- to 8-membered heterocyclyl, or —O—C₁₋₆ alkyl; wherein the C₁₋₆ alkyl, C₃₋₆ cycloalkyl, and 3- to 8-membered heterocyclyl of R^{g2} and R^{h2} are each optionally substituted with 1, 2, 3 or 4 substituents independently selected from R¹³;
   or R^{g2} and R^{h2} are taken together with the phosphorus atom to which they are attached to form a 4- to 6-membered heterocyclyl optionally substituted with 1, 2, 3 or 4 substituents independently selected from R¹³; and
each R¹³ is independently oxo, halogen, hydroxyl, —O(C₁₋₆ alkyl), cyano, C₁₋₆ alkyl or C₁₋₆ haloalkyl;
provided that the compound is other than a compound in Table 1X and salts thereof.
   Embodiment 2. The compound of Embodiment 1, wherein both G¹ and G² are N, and the compound is of the Formula (IA):

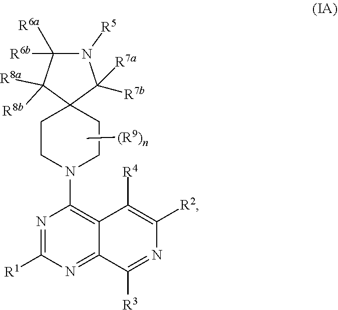

or a pharmaceutically acceptable salt thereof, wherein R¹, R², R³, R⁴, R⁵, R^{6a}, R^{6b}, R^{7a}, R^{7b}, R^{8a}R^{8b}, R⁹ and n are as defined in Embodiment 1.
Embodiment 3. The compound of Embodiment 1, wherein G¹ is N and G² is CR⁴², and the compound is of the Formula (IB):

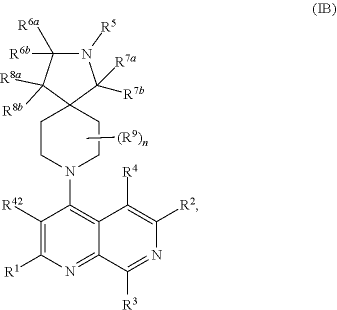

or a pharmaceutically acceptable salt thereof, wherein R¹, R², R³, R⁴, R⁴², R⁵, R^{6a}, R^{6b}, R^{7a}, R^{7b}, R^{8a}, R^{8b}, R⁹ and n are as defined in Embodiment 1.
Embodiment 4. The compound of Embodiment 1, wherein G¹ is CR⁴¹ and G² is N, and the compound is of the Formula (IC):

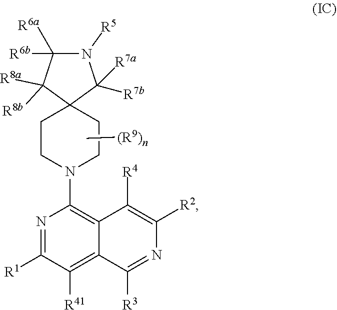

or a pharmaceutically acceptable salt thereof, wherein R¹, R², R³, R⁴, R⁴¹, R⁵, R^{6a}, R^{6b}, R^{7a}, R^{7b}, R^{1a}, R^{8b}, R⁹ and n are as defined in Embodiment 1.
Embodiment 5. The compound of any one of Embodiments 1 to 4, wherein R¹ is 6-membered heteroaryl having 1 or 2 ring nitrogen atoms, optionally substituted with 1, 2, 3 or 4 substituents independently selected from R¹⁰.
Embodiment 6. The compound of Embodiment 5, wherein R¹ is 4-pyridyl optionally substituted with 1 to 5 substituents independently selected from R¹⁰.
Embodiment 7. The compound of any one of Embodiments 1 to 4, wherein R¹ is 5-membered heteroaryl having 1 or 2 ring nitrogen atoms, optionally substituted with 1, 2, 3 or 4 substituents independently selected from R¹⁰.
Embodiment 8. The compound of Embodiment 7, wherein R¹ is pyrazol-4-yl optionally substituted with 1 to 3 substituents independently selected from R¹⁰.
Embodiment 9. The compound of any one of Embodiments 1 to 4, wherein R¹ is 5,6-fused heteroaryl having 1 or 2 ring nitrogen atoms, optionally substituted with 1, 2, 3, 4 or 5 substituents independently selected from R¹⁰.
Embodiment 10. The compound of any one of Embodiments 1 to 4, wherein R¹ is selected from the group consisting of.

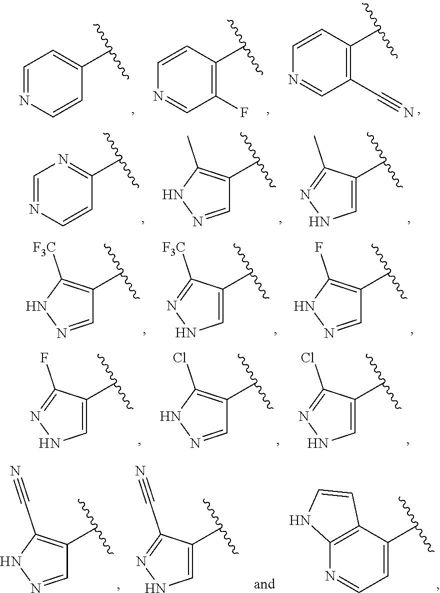

wherein the wavy line in each group indicates the point of attachment to the parent structure.
Embodiment 11. The compound of any one of Embodiments 1 to 10, wherein R² is hydrogen or C₁₋₆ alkyl optionally substituted with 1, 2, 3, 4 or 5 substituents independently selected from R¹⁰.
Embodiment 12. The compound of Embodiment 11, wherein R² is selected from the group consisting of hydrogen, methyl,

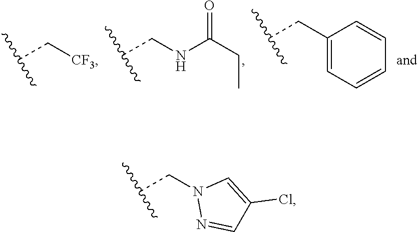

wherein the wavy line in each group indicates the point of attachment to the parent structure.
Embodiment 13. The compound of any one of Embodiments 1 to 12, wherein R³ is hydrogen, C₁₋₆ alkyl or C₁₋₆ haloalkyl.
Embodiment 14. The compound of Embodiment 13, wherein R³ is selected from the group consisting of hydrogen, methyl and 2,2,2-trifluoroethyl.
Embodiment 15. The compound of any one of Embodiments 1 to 14, wherein R⁴ is hydrogen, halogen, C₁₋₆ alkyl or C₃₋₆ cycloalkyl.
Embodiment 16. The compound of Embodiment 15, wherein R⁴ is selected from the group consisting of hydrogen, fluoro, chloro, methyl and cyclopropyl.
Embodiment 17. The compound of any one of Embodiments 1 to 16, wherein R⁵ is hydrogen, C₁₋₆ alkyl, C₃₋₈ cycloalkyl, C₆₋₁₄ aryl, 5- to 14-membered heteroaryl, 3- to 14-membered heterocyclyl or —C(O)R¹⁴, wherein the C₁₋₆ alkyl, C₃₋₈ cycloalkyl, C₆₋₁₄ aryl, 5- to 14-membered heteroaryl and 3- to 14-membered heterocyclyl of R⁵ are each optionally substituted with 1, 2, 3, 4 or 5 substituents independently selected from R¹⁰.
Embodiment 18. The compound of Embodiment 17, wherein R⁵ is hydrogen or —C(O)R¹⁴.
Embodiment 19. The compound of Embodiment 18, wherein R⁵ is hydrogen or acetyl.
Embodiment 20. The compound of Embodiment 17, wherein R⁵ is C₁₋₆ alkyl optionally substituted with 1, 2, 3, 4 or 5 substituents independently selected from R¹⁰.
Embodiment 21. The compound of Embodiment 20, wherein R⁵ is selected from the group consisting of methyl, ethyl, 1-propyl, 2-propyl, 2-methyl-1-propyl and 2-methyl-2-propyl,

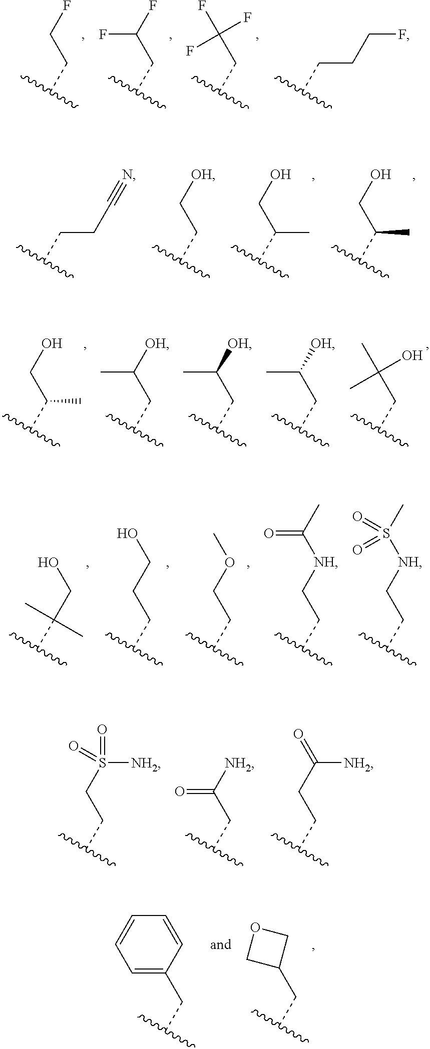

wherein the wavy line in each group indicates the point of attachment to the parent structure.
Embodiment 22. The compound of Embodiment 17, wherein R⁵ is C₄₋₈ cycloalkyl optionally substituted with 1, 2, 3, 4 or 5 substituents independently selected from R¹⁰.
Embodiment 23. The compound of Embodiment 22, wherein R⁵ is selected from the group consisting of

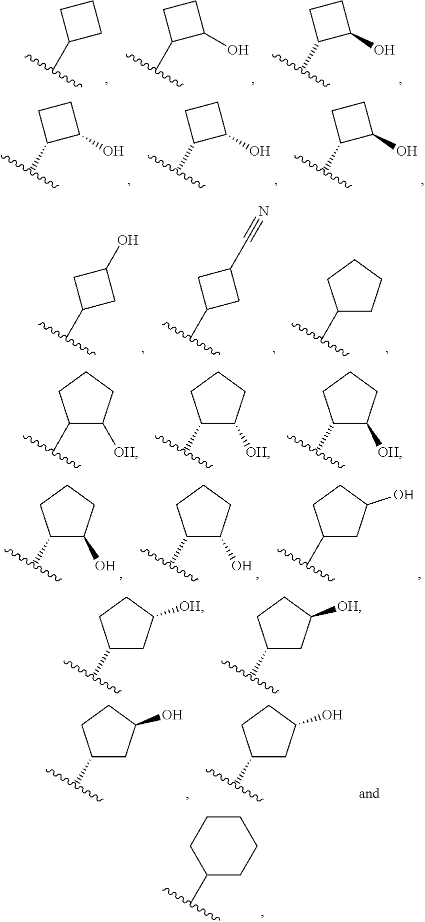

wherein the wavy line in each group indicates the point of attachment to the parent structure.
Embodiment 24. The compound of Embodiment 17, wherein R⁵ is 3- to 14-membered heterocyclyl optionally substituted with 1, 2, 3, 4 or 5 substituents independently selected from R¹⁰, C₆₋₁₄ aryl optionally substituted with 1, 2, 3, 4 or 5 substituents independently selected from R¹⁰, or 5- to 14-membered heteroaryl optionally substituted with 1, 2, 3, 4 or 5 substituents independently selected from R¹⁰.
Embodiment 25. The compound of Embodiment 24, wherein R⁵ is selected from the group consisting of

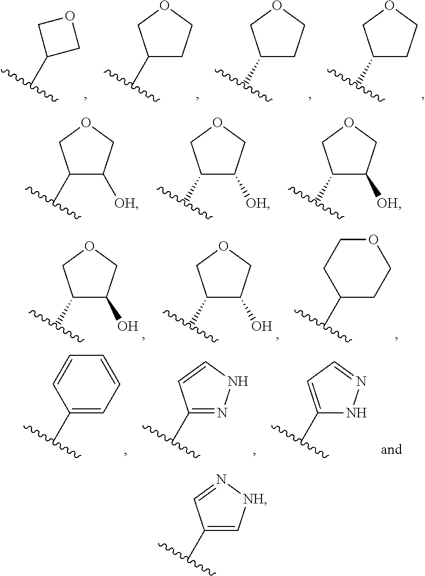

each of which is optionally substituted with 1, 2, 3, 4 or 5 substituents independently selected from R¹⁰, wherein the wavy line in each group indicates the point of attachment to the parent structure.
Embodiment 26. The compound of any one of Embodiments 1 to 25, wherein each R^{6a} and R^{6b} is independently hydrogen or C₁₋₆ alkyl; or R^{6a} and R^{6b} are taken together with the carbon to which they are attached to form a carbonyl.
Embodiment 27. The compound of any one of Embodiments 1 to 25, wherein one of R^{6a} and R^{6b} is hydrogen and the other one of R^{6a} and R^{6b} is hydrogen, —C(O)OR¹⁵, —C(O)NR^{16a}R^{16b} or C₁₋₆ alkyl optionally substituted with 1, 2, 3, 4 or 5 substituents independently selected from R¹⁰.
Embodiment 28. The compound of Embodiment 27, wherein one of R^{6a} and R^{6b} is hydrogen and the other one of R^{6a} and R^{6b} is —C(O)OR¹⁵ or —C(O)NR^{16a}R^{16b}; wherein each R^{16a} and R^{16b} is independently hydrogen or C₁₋₆ alkyl.
Embodiment 29. The compound of Embodiment 27, wherein one of R^{6a} and R^{6b} is hydrogen and the other one of R^{6a} and R^{6b} is —C(O)NR^{16a}R^{16b}; wherein R^{16a} and R^{16b} are taken together with the nitrogen atom to which they are attached to form a 4- to 12-membered heterocyclyl optionally substituted with 1, 2, 3, 4 or 5 substituents independently selected from R¹⁰.
Embodiment 30. The compound of Embodiment 27, wherein one of R^{6a} and R^{6b} is hydrogen and the other one of R^{6a} and R^{6b} is selected from the group consisting of hydrogen, methyl,

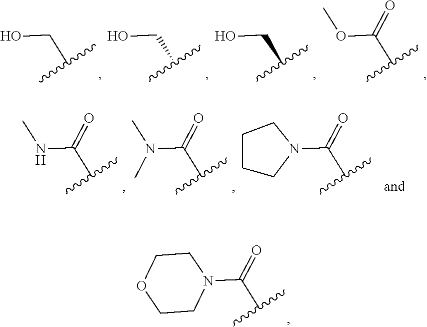

wherein the wavy line in each group indicates the point of attachment to the parent structure.
Embodiment 31. The compound of any one of Embodiments 1 to 30, wherein each R^{7a} and R^{7b} is independently hydrogen or C₁₋₆ alkyl; or R^{7a} and R^{7b} are taken together with the carbon to which they are attached to form a carbonyl.
Embodiment 32. The compound of any one of Embodiments 1 to 31, wherein one of R^{8a}, and R^{8b} is hydrogen, and the other one of R^{8a} and R^{8b} is hydrogen, halogen, hydoxyl, C₁₋₆ alkyl, or —O(C₁₋₆ alkyl).
Embodiment 33. The compound of Embodiment 32, wherein each R^{8a} and R^{8b} is hydrogen.
Embodiment 34. The compound of any one of Embodiments 1 to 33, wherein n is 0.
Embodiment 35. The compound of any one of Embodiments 1 to 34, wherein the compound is of the Formula (II):

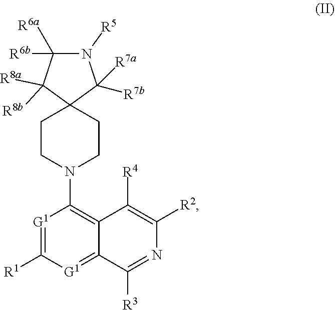

or a pharmaceutically acceptable salt thereof, wherein R¹, R², R³, R⁴, G¹, G², R⁵, R^{6a}, R^{6b}, R^{7a}, R^{7b}, R^{8a} and R^{8b} are as defined in any one of Embodiments 1 to 30.
Embodiment 36. The compound of Embodiment 35, wherein both G¹ and G² are N, and the compound is of the Formula (II-A):

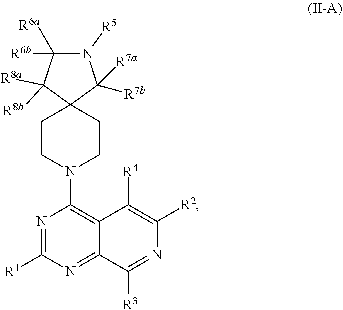

or a pharmaceutically acceptable salt thereof, wherein R¹, R², R³, R⁴, R⁵, R^{6a}, R^{6b}, R^{7a}, R^{7b}, R^{8a}, and R^{8b} are as defined in Embodiment 35.
Embodiment 37. The compound of Embodiment 35, wherein G¹ is N and G² is CR⁴², and the compound is of the Formula (II-B):

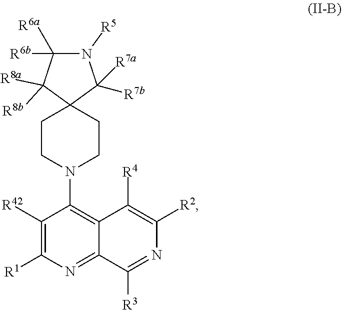

or a pharmaceutically acceptable salt thereof, wherein R¹, R², R³, R⁴, R⁴², R⁵, R^{6a}, R^{6b}, R^{7a}, R^{7b}, R^{8a} and R^{8b} are as defined in Embodiment 35.
Embodiment 38. The compound of Embodiment 37, wherein R⁴² is hydrogen.
Embodiment 39. The compound of Embodiment 35, wherein G¹ is CR⁴¹ and G² is N, and the compound is of the Formula (II-C):

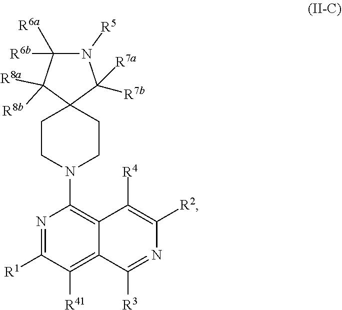

or a pharmaceutically acceptable salt thereof, wherein R¹, R², R³, R⁴, R⁴¹, R⁵, R^{6a}, R^{6b}, R^{7a}, R^{7b}, R^{8a} and R^{8b} are as defined in Embodiment 35.
Embodiment 40. The compound of Embodiment 39, wherein R⁴¹ is hydrogen.
Embodiment 41. The compound of any one of Embodiments 35 to 40, wherein:
R¹ is pyrazolyl, pyridinyl or pyrrolo-pyridinyl, each of which is optionally substituted with 1 to 3 substituents independently selected from R¹⁰;
R² is hydrogen or C₁₋₆ alkyl optionally substituted with 1 to 5 substituents independently selected from R¹⁰;
R³ is hydrogen or C₁₋₆ alkyl;
R⁴ is hydrogen, halogen or C₁₋₆ alkyl;
R⁵ is hydrogen, C₁₋₆ alkyl, C₃₋₈ cycloalkyl, C₆₋₁₄ aryl, 5- to 14-membered heteroaryl, 3- to 14-membered heterocyclyl or —C(O)R¹⁴, wherein the C₁₋₆ alkyl, C₃₋₈ cycloalkyl, C₆₋₁₄ aryl, 5- to 14-membered heteroaryl and 3- to 14-membered heterocyclyl of R⁵ are each optionally substituted with 1, 2, 3, 4 or 5 substituents independently selected from R¹⁰;
each R^{6a} and R^{6b} is independently hydrogen, —C(O)OR¹⁵, —C(O)NR^{16a}R^{16b} or C₁₋₆ alkyl optionally substituted with 1, 2, 3, 4 or 5 substituents independently selected from R¹⁰, or R 6a and R^{6b} are taken together with the carbon to which they are attached to form a carbonyl;
each R^{7a} and R^{7b} is independently hydrogen or C₁₋₆ alkyl, or R^{7a} and R^{7b} are taken together with the carbon to which they are attached to form a carbonyl;
one of R^{8a} and R^{8b} is hydrogen, and the other one of R^{8a} and R^{8b} is hydrogen, halogen, hydoxyl, C₁₋₆ alkyl, or —O(C₁₋₆ alkyl);
R¹⁴ is C₁₋₆ alkyl;
R¹⁵ is C₁₋₆ alkyl; and
each R^{16a} and R^{16b} is independently hydrogen or C₁₋₆ alkyl, or R^{16a} and R^{16b} are taken together with the nitrogen atom to which they are attached to form a 5- or 6-membered heterocyclyl having 1 to 2 annular heteroatoms selected from nitrogen, oxygen and sulfur, optionally substituted with 1, 2, 3, 4 or 5 substituents independently selected from R¹⁰.
   Embodiment 42. The compound of any one of Embodiments 35 to 40, wherein:
R¹ is pyrazol-4-yl, 4-pyridyl or pyrrolo[2,3-b]pyridin-4-yl, each of which is optionally substituted with 1 to 3 substituents independently selected from the group consisting of halogen, cyano, unsubstituted C₁₋₆ alkyl and C₁₋₆ haloalkyl;
each R² and R³ is independently hydrogen or C₁₋₆ alkyl;
R⁴ is hydrogen, halogen or C₁₋₆ alkyl;
R⁵ is (i) C₁₋₆ alkyl optionally substituted with 1, 2, 3, 4 or 5 substituents independently selected from the group consisting of halogen, cyano, hydroxyl, —O(C₁₋₆ alkyl), —NHC(O)(C₁₋₆ alkyl), —NHS(O)₂(C₁₋₆ alkyl), —S(O)₂NH₂, —C(O)NH₂, phenyl and 3- to 12-membered heterocyclyl,
(ii) C₃₋₆ cycloalkyl substituted with 1, 2, 3, 4 or 5 substituents independently selected from the group consisting of halogen, cyano and hydroxyl,
(iii) monocyclic 3- to 6-membered heterocyclyl having 1 annular heteroatom which is oxygen,
(iv) phenyl, or
(v) pyrazolyl;
one of R^{6a} and R^{6b} is hydrogen, and the other one of R^{6a} and R^{6b} is hydrogen, C₁₋₆ alkyl, —C(O)O(C₁₋₆ alkyl) or —C(O)NR^{16a}R^{16b}, or R^{6a} and R^{6b} are taken together with the carbon to which they are attached to form a carbonyl;
one of R^{7a} and R^{7b} is hydrogen, and the other one of R^{7a} and R^{7b} is hydrogen or C₁₋₆ alkyl, or R^{7a} and R^{7b} are taken together with the carbon to which they are attached to form a carbonyl;
R^{8a} and R^{8b} are hydrogen; and
each R^{16a} and R^{16b} is independently hydrogen or C₁₋₆ alkyl, or R^{16a} and R^{16b} are taken together with the nitrogen atom to which they are attached to form pyrrolidin-1-yl or morpholin-4-yl.
   Embodiment 43. The compound of any one of Embodiments 1 to 34, wherein the compound is of the Formula (III):

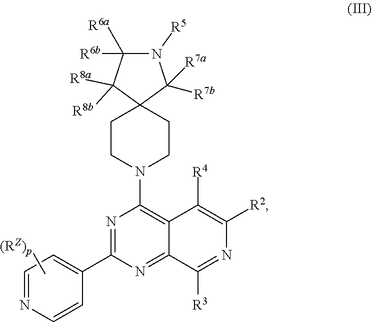

or a pharmaceutically acceptable salt thereof, wherein R², R³, R⁴, R⁵, R^{6a}, R^{6b}, R^{7a}, R^{7b}, R^{8a} and R^{8b} are as defined, where applicable, in any one of Embodiments 1 to 30;
p is 0, 1, 2, 3 or 4; and
each R^{Z} is independently hydrogen, halogen, cyano or C₁₋₆ alkyl optionally substituted with 1, 2, 3, 4 or 5 substituents independently selected from R¹⁰.
   Embodiment 44. The compound of Embodiment 43, wherein p is 0, and the compound is of the Formula (V):

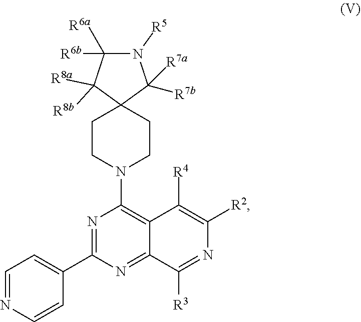

or a pharmaceutically acceptable salt thereof.
Embodiment 45. The compound of any one of Embodiments 1 to 34, wherein the compound is of the Formula (IV):

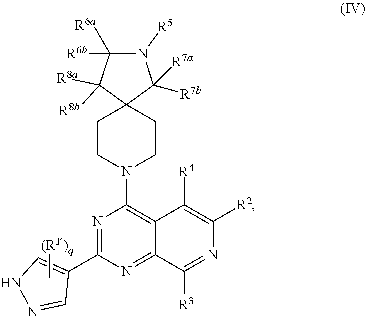

or a pharmaceutically acceptable salt thereof, wherein R², R³, R⁴, R⁵, R^{6a}, R^{6b}, R^{7a}, R^{7b}, R^{8a} and R^{8b} are as defined, where applicable, in any one of Embodiments 1 to 30;
q is 0, 1, 2 or 3; and
each R^{Y} is independently hydrogen, halogen, cyano, —O(C₁₋₆ alkyl) or C₁₋₆ alkyl, wherein the C₁₋₆ alkyl of R is optionally substituted with 1, 2, 3, 4 or 5 substituents independently selected from R¹⁰.
   Embodiment 46. The compound of Embodiment 45, wherein q is 1, and R^{Y} is methyl, fluoro, chloro, cyano or trifluoromethyl.
   Embodiment 47. The compound of any one of Embodiments 1 to 34, wherein the compound is of the Formula (VII) or (VIII):

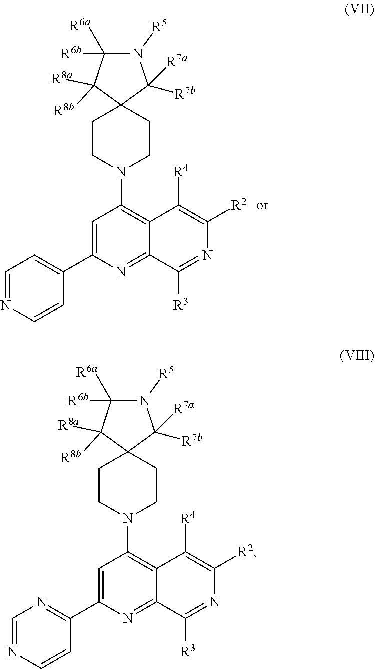

or a pharmaceutically acceptable salt thereof, wherein R², R³, R⁴, R⁵, R^{6a}, R^{6b}, R^{7a}, R^{7b}, R^{8a} and R^{8b} are as defined, where applicable, in any one of Embodiments 1 to 34.
Embodiment 48. The compound of any one of Embodiments 1 to 34, wherein the compound is of the Formula (IX):

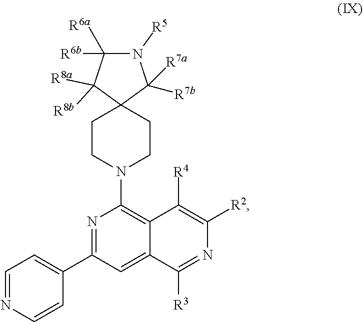

or a pharmaceutically acceptable salt thereof, wherein R², R³, R⁴, R⁵, R^{6a}, R^{6b}, R^{7a}, R⁷, R^{8a} and R^{8b} are as defined, where applicable, in any one of Embodiments 1 to 34.
Embodiment 49. The compound of any one of Embodiments 1 to 48, wherein each R², R³ and R⁴ is hydrogen.
Embodiment 50. The compound of Embodiment 1, wherein the compound is selected from the group consisting of Compound Nos. 101 to 201 in Table 1, or a pharmaceutically acceptable salt thereof.
Embodiment 51. A pharmaceutical composition comprising the compound of any one of Embodiments 1 to 50, or a pharmaceutically acceptable salt thereof; and a pharmaceutically acceptable excipient.
Embodiment 52. A method for making a compound of Formula (I):

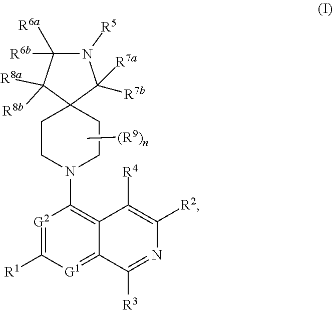

or a pharmaceutically acceptable salt thereof, wherein R¹, R², R³, R⁴, G¹, G², R⁵, R^{6a}, R^{6b}, R^{7a}R^{7b}, R^{8a}, R^{8b}, R⁹, and n are as defined in Embodiment 1, the method comprising
a) reacting a compound of Formula (I-4):

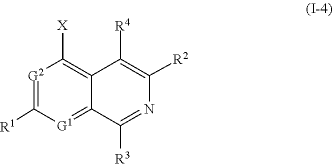

wherein X is selected from the group consisting of Cl, Br, I, F, OMs, and OTs, with a compound of Formula (I-3):

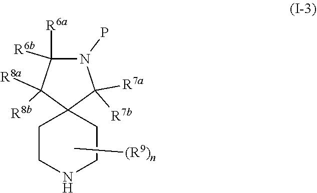

wherein P is a protecting group, in the presence of a base to produce a compound of Formula (I-2):

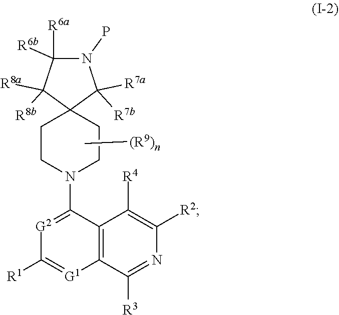

b) removing protecting group P from the compound of Formula (I-2) to produce a compound of Formula (I-1):

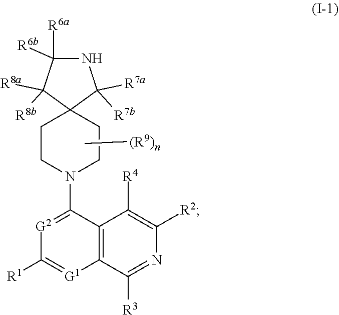

and
c) converting the compound of Formula (I-1) to the compound of Formula (I).
   Embodiment 53. The method of Embodiment 52, wherein the compound of Formula (I-1) is converted to the compound of Formula (I) (i) by contacting the compound of Formula (I-1) with a compound of the formula R⁵—X′, wherein X′ is a leaving group, in the presence of a base; or (ii) by contacting the compound of Formula (I-1) with an aldehyde of the formula R⁵—CHO in the presence of a reducing agent.
   Embodiment 54. The method of Embodiment 52, wherein both G¹ and G² are N, and the method further comprising:
d) mixing a compound of Formula 1

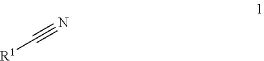

with a compound of Formula 2:

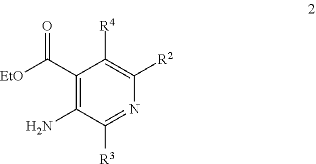

in the presence of a base to form a compound of Formula 3:

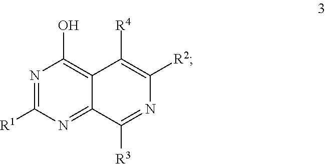

and
e) contacting the compound of Formula 3 with an activating agent to form the compound of Formula (I-4) wherein both G¹ and G² are N.
   Embodiment 55. A method for making a compound of formula (IA):

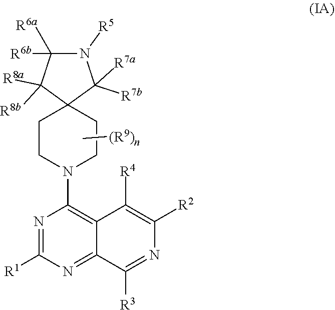

or a pharmaceutically acceptable salt thereof, wherein R¹, R², R³, R⁴, R⁵, R^{6a}, R^{6b}, R^{7a}, R^{7b}, R^{8a}, R^{8b}, R⁹, and n are as defined in Embodiment 1, the method comprising:
a) contacting a compound of Formula 12:

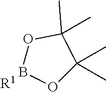

with a compound of Formula 11:

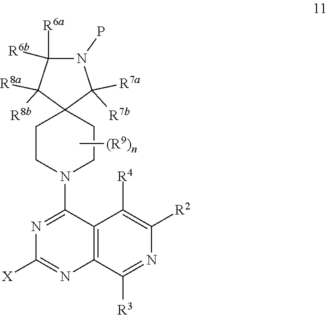

in the presence of a palladium catalyst and a base to form a compound of Formula 6:

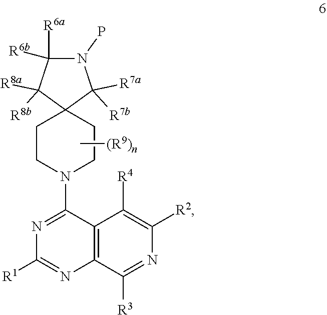

wherein X is selected from the group consisting of Cl, Br, I, F, OMs, and OTs, and P is a protecting group;
b) removing protecting group P from the compound of Formula 6 to produce a compound of Formula 7:

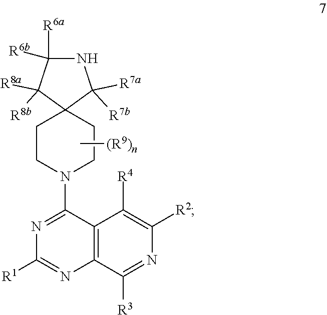

and
c) converting the compound of Formula 7 to the compound of Formula (IA).
   Embodiment 56. The method of Embodiment 55 further comprising:
d) coupling a compound of Formula 5:

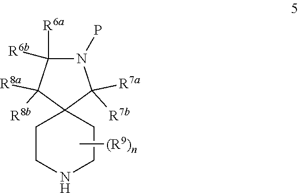

to a compound of Formula 10:

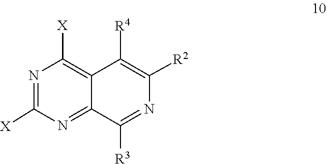

wherein X is selected from the group consisting of Cl, Br, I, F, OMs, and OTs, in the presence of a base to form the compound of Formula 11.
   Embodiment 57. The method of Embodiment 55 or 56, wherein the compound of Formula 7 is converted to the compound of Formula (IA) (i) by contacting the compound of Formula 7 with a compound of Formula 8:

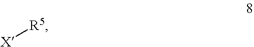

wherein X′ is a leaving group, in the presence of a base; or
(ii) by contacting the compound of Formula 7 with a compound of Formula 9 in the presence of a reducing agent:

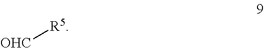

Embodiment 58. A method of inhibiting LATS1/2 in a cell, comprising contacting the cell with the compound of any one of Embodiments 1 to 50, or a pharmaceutically acceptable salt thereof, or the pharmaceutical composition of Embodiment 51.
Embodiment 59. A method for treating a disease or condition, said method comprising administering to a subject in need thereof an effective amount of the compound of any one of Embodiments 1 to 50, or a pharmaceutically acceptable salt thereof, or the pharmaceutical composition of Embodiment 51.
Embodiment 60. The method of Embodiment 54, wherein the disease or condition is acute respiratory distress syndrome (ARDS).

### EXAMPLES

Abbreviations
AcOH—acetic acid
Boc—tert-butyloxycarbonyl
Cbz—carboxybenzyl
DBU—1,8-diazabicyclo[5.4.0]undec-7-ene
DCM—dichloromethane
DIEA or DIPEA—N,N-diisopropylethylamine
DMA—dimethylacetamide
DMF—dimethylformamide
DMSO—dimethyl sulfoxide
DTT—dithiothreitol
EtOAc—ethyl acetate
EtOH—ethanol
Fmoc—fluorenylmethyloxycarbonyl
HATU—1-[bis(dimethylamino)methylene]-1H-1,2,3-triazolo[4,5-b]pyridinium 3-oxid hexafluorophosphate
HEPES—4-(2-hydroxyethyl)-1-piperazineethanesulfonic acid
LiHMDS—Lithium bis(trimethylsilyl)amide
MeCN—acetonitrile
MeOH—methanol
Ms—mesyl
NaBH(OAc)₃—sodium triacetoxyborohydride
NBS—N-bromosuccinimide
NMO—N-methylmorpholine-N-oxide
Pd(PPh₃)₄—Tetrakis(triphenylphosphine)palladium(0)
SEM—(2-(trimethylsilyl)ethoxy)methyl
SFC—supercritical fluid chromatography
TBS—tert-butyldimethylsilyl
TBSCl—tert-butyldimethylsilyl chloride
TFA—trifluoroacetic acid
THF—tetrahydrofuran
Ts—tosyl

### SYNTHETIC EXAMPLES

### Example 101

### 2-(pyridin-4-yl)-4-(2,8-diazaspiro[4.5]decan-8-yl)pyrido[3,4-d]pyrimidine (Compound 101)

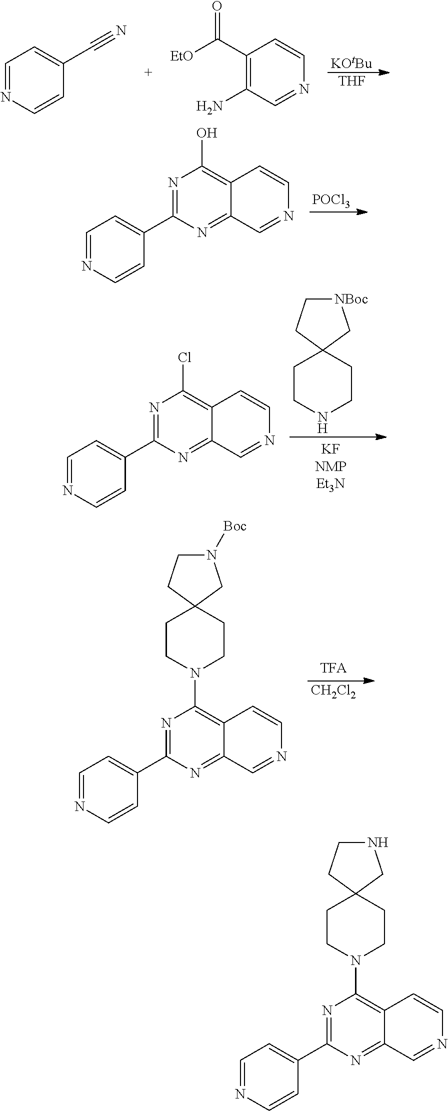

### Step 1: 2-(pyridin-4-yl)pyrido[3,4-d]pyrimidin-4-ol

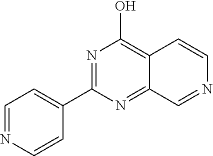 [SMILES: Oc1nc(-c2ccncc2)nc2cnccc12]

To a solution of potassium 2-methyl-2-butoxide (12.45 g, 98.59 mmol) in THF (80 mL) was added a solution of ethyl methyl 3-aminoisonicotinate (6.0 g, 39.43 mmol) and 4-cyanopyridine (4.93 g, 47.32 mmol) in THF (80 mL) dropwise (˜4 mL/min) at 0° C. The reaction was allowed to warm to room temperature and stirred for 16 hours. Water (50 mL) and acetic acid (15 mL) were added. The mixture was stirred at room temperature for 20 minutes, the resulting yellow precipitate was filtered and the solid was washed with water (30 mL×2) to give the title compound (5 g, 49%) as a yellow solid. ¹H NMR (400 MHz, DMSO-d₆) δ 13.08 (s, 1H), 9.15 (s, 1H), 8.81 (d, J=6.0 Hz, 2H), 8.70 (d, J=5.2 Hz, 1H), 8.11 (d, J=6.0 Hz, 2H), 8.00 (d, J=5.2 Hz, 1H). LCMS (ESI) m/z: 225.2 [M+H]+.

### Step 2: 4-chloro-2-(pyridin-4-yl)pyrido[3,4-d]pyrimidine

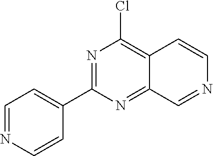 [SMILES: Clc1nc(-c2ccncc2)nc2cnccc12]

A solution of 2-(pyridin-4-yl)pyrido[3,4-d]pyrimidin-4-ol (2.5 g, 11.15 mmol) in phosphorus oxychloride (17 mL, 182.38 mmol) was heated to 110° C. for 16 hours. After cooling to room temperature, the mixture was concentrated in vacuo. The crude residue was dissolved in DCM (200 mL) and basified with sat. aq. NaHCO₃ (100 mL) to pH 8 at 0° C. The organic layer was dried over anhydrous Na₂SO₄, filtered and concentrated in vacuo to give the title compound (2.4 g, crude) as a brown solid. ¹H NMR (400 MHz, DMSO-d₆) δ 9.64 (s, 1H), 8.95 (d, J=5.6 Hz, 1H), 8.88-8.85 (m, 2H), 8.39-8.34 (m, 2H), 8.18 (m, J=5.2 Hz, 1H). LCMS (ESI) m/z: 242.9 [M+H]+.

### Step 3: 2-(pyridin-4-yl)-4-(2,8-diazaspiro[4.5]decan-8-yl)pyrido[3,4-d]pyrimidine

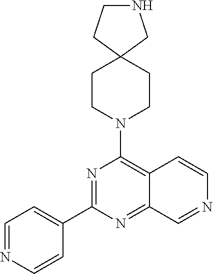 [SMILES: c1cc(-c2nc(N3CCC4(CCNC4)CC3)c3ccncc3n2)ccn1]

To a 2-dram vial was added 4-chloro-2-(4-pyridyl)pyrido[3,4-d]pyrimidine (300 mg, 1.236 mmol, 1 equiv.), potassium fluoride (215 mg, 3.71 mmol, 3 equiv.), followed by 1-methyl-2-pyrrolidinone (4.10 mL, 0.3 M), triethylamine (0.862 mL, 6.18 mmol, 5 equiv.), and tert-butyl 2,8-diazaspiro[4.5]decane-2-carboxylate (310 mg, 1.24 mmol, 1 equiv.). The reaction was allowed to stir at room temperature for 2 hours. The reaction was then quenched via the addition of water (3 mL), diluted with EtOAc (5 mL). Layers separated; organics washed with water (3×3 mL) followed by brine (2×3 mL). The organic layer was dried over Na₂SO₄, filtered, and concentrated in vacuo. The crude residue was further concentrated on the Genevac for 16 hours to remove residual DMSO. To the crude residue was then added 1 mL DCM followed by 0.5 mL TFA. The mixture was allowed to stir for 4 hours at room temperature and then the was concentrated in vacuo, and then concentrated 2× further from DCM (5 mL) to remove as much residual TFA as possible. The crude residue was then purified by HPLC to fumish 4-(2,8-diazaspiro[4.5]decan-8-yl)-2-(4-pyridyl)pyrido[3,4-d]pyrimidine (348 mg, 1.00 mmol, 81% Yield). ¹H NMR (400 MHz, DMSO) δ 9.26 (s, 1H), 8.79-8.74 (m, 2H), 8.59 (d, J=5.6 Hz, 1H), 8.35-8.29 (m, 2H), 7.90 (d, J=5.7 Hz, 1H), 4.02-3.88 (m, 4H), 2.85 (t, J=7.0 Hz, 2H), 2.67 (s, 2H), 1.72 (t, J=5.7 Hz, 4H), 1.61 (t, J=7.1 Hz, 2H). Exchangeable amine NH proton not observed. LCMS (ESI) m/z: 347.2 [M+H]+.

### Example 102

### 4-(2-methyl-2,8-diazaspiro[4.5]decan-8-yl)-2-(pyridin-4-yl)pyrido[3,4-d]pyrimidine (Compound 102)

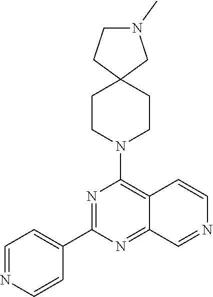 [SMILES: CN1CCC2(CCN(c3nc(-c4ccncc4)nc4cnccc34)CC2)C1]

To a solution of 4-(2,8-diazaspiro[4.5]decan-8-yl)-2-(4-pyridyl)pyrido[3,4-d]pyrimidine hydrochloride (100 mg, 0.26 mmol) in 1,2-dichloroethane (3 mL) was added formaldehyde (106 mg, 1.31 mmol, 37% in water) and acetic acid (0.03 mL, 0.52 mmol). The mixture was stirred at room temperature for 10 minutes before the addition of sodium triacetoxyborohydride (277 mg, 1.31 mmol). The mixture was stirred at room temperature for 16 hours. The reaction mixture was concentrated in vacuo. The crude residue was dissolved in EtOAc (20 mL), washed with sat. aq. NaHCO₃ (10 mL) and brine (10 mL). The organic layer was dried over anhydrous Na₂SO₄, filtered and concentrated in vacuo. The crude residue was purified by reverse phase chromatography (acetonitrile 35-65%/0.05% NH₃·H₂O+10 mM NH₄HCO₃ in water) to give the title compound (10 mg, 10%) as a white solid. ¹H NMR (400 MHz, DMSO-d₆) δ 9.25 (s, 1H), 8.79-8.74 (m, 2H), 8.58 (d, J=5.6 Hz, 1H), 8.35-8.29 (m, 2H), 7.88 (d, J=6.0 Hz, 1H), 4.03-3.93 (m, 2H), 3.93-3.82 (m, 2H), 2.53-2.48 (m, 2H), 2.39 (s, 2H), 2.23 (s, 3H), 1.78-1.66 (m, 6H). LCMS (ESI) m/z: 361.2 [M+H]+.

### Examples 103 and 104

### (R)-2-(8-(2-(pyridin-4-yl)pyrido[3,4-d]pyrimidin-4-yl)-2,8-diazaspiro[4.5]decan-2-yl)propan-1-ol and (S)-2-(8-(2-(pyridin-4-yl)pyrido[3,4-d]pyrimidin-4-yl)-2,8-diazaspiro[4.5]decan-2-yl)propan-1-ol (Compounds 103 and 104)

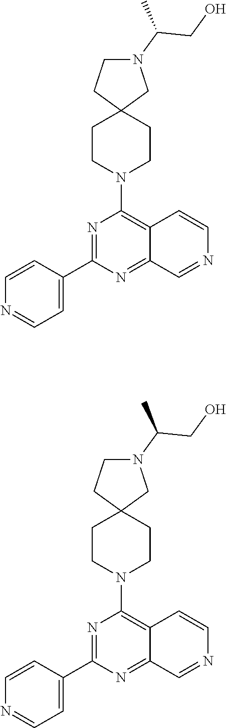 [SMILES: C[C@H](CO)N1CCC2(CCN(c3nc(-c4ccncc4)nc4cnccc34)CC2)C1 ; C[C@@H](CO)N1CCC2(CCN(c3nc(-c4ccncc4)nc4cnccc34)CC2)C1]

### Step 1: 2-(8-(2-(pyridin-4-yl)pyrido[3,4-d]pyrimidin-4-yl)-2,8-diazaspiro[4.5]decan-2-yl)propan-1-ol

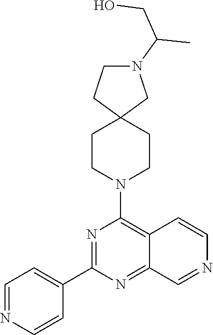 [SMILES: CC(CO)N1CCC2(CCN(c3nc(-c4ccncc4)nc4cnccc34)CC2)C1]

To a solution of 4-(2,8-diazaspiro[4.5]decan-8-yl)-2-(4-pyridyl)pyrido[3,4-d]pyrimidine hydrochloride (110 mg, 0.29 mmol) in MeOH (4 mL) was added N,N-diisopropylethylamine (0.11 mL, 0.58 mmol). The reaction mixture was stirred at room temperature for 5 minutes, acetic acid (0.03 mL, 0.52 mmol) and hydroxyacetone (0.06 mL, 0.81 mmol) were added to this mixture. The mixture was stirred at room temperature for 20 minutes before the addition of sodium cyanoborohydride (60 mg, 0.95 mmol). The mixture was stirred at room temperature for 16 hours. The reaction mixture was concentrated in vacuo. The crude residue was purified by reverse phase chromatography (acetonitrile 10-40%/0.225% formic acid in water) to give the title compound (50 mg, 43%) as a yellow solid. LCMS (ESI) m/z: 405.3 [M+H]+.

### Step 2: (R)-2-(8-(2-(pyridin-4-yl)pyrido[3,4-d]pyrimidin-4-yl)-2,8-diazaspiro[4.5]decan-2-yl)propan-1-ol and (S)-2-(8-(2-(pyridin-4-yl)pyrido[3,4-d]pyrimidin-4-yl)-2,8-diazaspiro[4.5]decan-2-yl)propan-1-ol

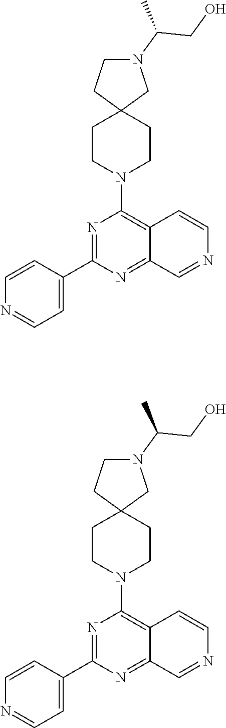 [SMILES: C[C@H](CO)N1CCC2(CCN(c3nc(-c4ccncc4)nc4cnccc34)CC2)C1 ; C[C@@H](CO)N1CCC2(CCN(c3nc(-c4ccncc4)nc4cnccc34)CC2)C1]

2-(8-(2-(pyridin-4-yl)pyrido[3,4-d]pyrimidin-4-yl)-2,8-diazaspiro[4.5]decan-2-yl)propan-1-ol (50 mg, 0.12 mmol) was separated by using chiral SFC (Chiralpak AD (250 mm*30 mm, 10 um), Supercritical CO₂/EtOH+0.1% NH₄OH=45/55; 60 mL/min) to give the title compounds, both as white solids. Absolute configuration was arbitrarily assigned to each enantiomer. Example 103 (3.9 mg, second peak): ¹H NMR (400 MHz, CD₃OD) δ 9.24 (s, 1H), 8.70 (d, J=5.6 Hz, 2H), 8.54 (d, J=5.6 Hz, 1H), 8.45 (d, J=6.0 Hz, 2H), 7.92 (d, J=5.2 Hz, 1H), 4.15-4.06 (m, 2H), 4.02-3.92 (m, 2H), 3.71-3.59 (m, 2H), 3.12-3.02 (m, 2H), 2.99-2.88 (m, 2H), 2.80-2.70 (m, 1H), 1.97-1.93 (m, 2H), 1.92-1.86 (m, 4H), 1.28-1.23 (m, 3H).LCMS (ESI) m/z: 405.1 [M+H]+. Example 104 (3.9 mg, first peak): ¹H NMR (400 MHz, CD₃OD) δ 9.23 (s, 1H), 8.70 (d, J=6.4 Hz, 2H), 8.54 (d, J=5.6 Hz, 1H), 8.44 (d, J=6.0 Hz, 2H), 7.91 (d, J=5.6 Hz, 1H), 4.15-4.05 (m, 2H), 4.01-3.90 (m, 2H), 3.71-3.59 (m, 2H), 3.12-3.02 (m, 2H), 2.98-2.88 (m, 2H), 2.80-2.70 (m, 1H), 1.97-1.92 (m, 2H), 1.91-1.87 (m, 4H), 1.28-1.22 (m, 3H). LCMS (ESI) m/z: 405.1 [M+H]+.

### Example 105

### 2-(pyridin-4-yl)-4-(2-(2,2,2-trifluoroethyl)-2,8-diazaspiro[4.5]decan-8-yl)pyrido[3,4-d]pyrimidine (Compound 105)

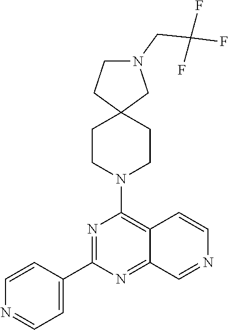 [SMILES: FC(F)(F)CN1CCC2(CCN(c3nc(-c4ccncc4)nc4cnccc34)CC2)C1]

To a solution of 4-(2,8-diazaspiro[4.5]decan-8-yl)-2-(4-pyridyl)pyrido[3,4-d]pyrimidine hydrochloride (30 mg, 0.09 mmol) in DMF (1 mL) was added N,N-diisopropylethylamine (0.05 mL, 0.26 mmol) and 2,2,2-trifluoroethyl trifluoromethanesulfonate (24 mg, 0.1 mmol). The reaction mixture was stirred at room temperature for 1 hour. The reaction mixture was concentrated in vacuo, the resulting residue was purified by reverse phase chromatography (acetonitrile 7-37%/0.225% formic acid in water) to give the title compound (11 mg, 28%) as a yellow solid. ¹H NMR (400 MHz, DMSO-d₆) δ 9.25 (s, 1H), 8.76 (d, J=6.0 Hz, 2H), 8.58 (d, J=6.0 Hz, 1H), 8.33-8.31 (m, 2H), 7.89 (d, J=6.0 Hz, 1H), 4.03-3.96 (m, 2H), 3.92-3.84 (m, 2H), 3.33-3.28 (m, 2H), 2.80 (t, J=6.8 Hz, 2H), 2.67 (s, 2H), 1.82-1.71 (m, 6H). LCMS (ESI) m/z: 429.1 [M+H]+.

### Example 106

### 4-(2-cyclopentyl-2,8-diazaspiro[4.5]decan-8-yl)-2-(pyridin-4-yl)pyrido[3,4-d]pyrimidine formate (Compound 106)

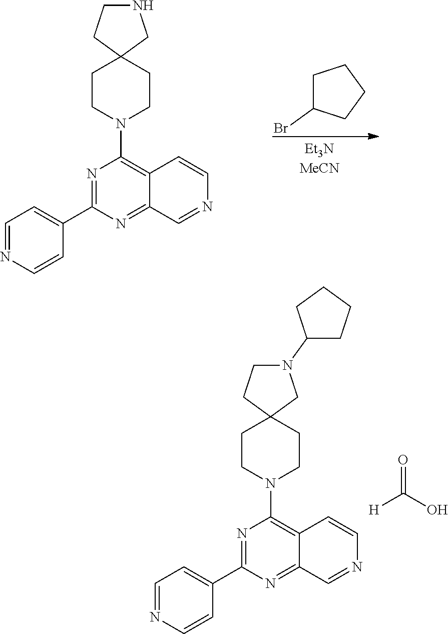

To a solution of 4-(2,8-diazaspiro[4.5]decan-8-yl)-2-(4-pyridyl)pyrido[3,4-d]pyrimidine hydrochloride (54 mg, 0.14 mmol) in acetonitrile (2 mL) was added triethylamine (0.06 mL, 0.43 mmol) and cyclopentyl bromide (0.03 mL, 0.29 mmol). The mixture was heated to 50° C. for 6 hours under nitrogen atmosphere. After cooling to room temperature, the mixture was diluted with EtOAc (30 mL), washed with water (20 mL) and brine (20 mL). The organic layer was dried over anhydrous Na₂SO₄, filtered and concentrated in vacuo. The crude residue was purified by reverse phase chromatography (acetonitrile 1-30%/0.225% formic acid in water) to give the title compound (8.3 mg, 12%) as a white solid. ¹H NMR (400 MHz, DMSO-d₆) δ 9.27 (s, 1H), 8.78 (d, J=6.0 Hz, 2H), 8.60 (d, J=5.6 Hz, 1H), 8.33 (d, J=6.0 Hz, 2H), 8.23 (s, 1H), 7.89 (d, J=6.0 Hz, 1H), 4.03-3.99 (m, 2H), 3.91-3.85 (m, 2H), 2.73 (t, J=6.8 Hz, 2H), 2.63-2.60 (m, 3H), 1.82-1.72 (m, 8H), 1.67-1.61 (m, 2H), 1.53-1.43 (m, 4H). LCMS (ESI) m/z: 415.2 [M+H]+.

### Example 107

### 2-methyl-2-(8-(2-(pyridin-4-yl)pyrido[3,4-d]pyrimidin-4-yl)-2,8-diazaspiro[4.5]decan-2-yl)propan-1-ol (Compound 107) formate

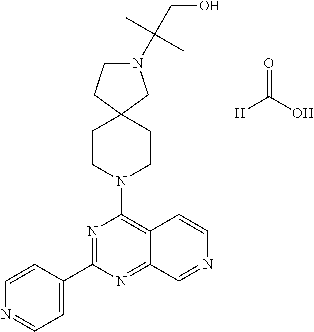 [SMILES: CC(C)(CO)N1CCC2(CCN(c3nc(-c4ccncc4)nc4cnccc34)CC2)C1 ; O=CO]

### Step 1: methyl 2-methyl-2-(8-(2-(pyridin-4-yl)pyrido[3,4-d]pyrimidin-4-yl)-2,8-diazaspiro[4.5]decan-2-yl)propanoate

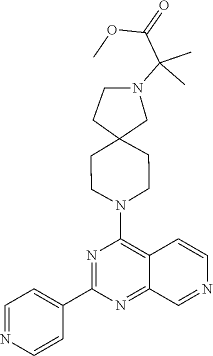 [SMILES: COC(=O)C(C)(C)N1CCC2(CCN(c3nc(-c4ccncc4)nc4cnccc34)CC2)C1]

To a solution of 4-(2,8-diazaspiro[4.5]decan-8-yl)-2-(4-pyridyl)pyrido[3,4-d]pyrimidine hydrochloride (150 mg, 0.39 mmol) in MeOH (3.9 mL) was added N,N-diisopropylethylamine (0.34 mL, 1.96 mmol) and methyl 2-bromo-2-methylpropanoate (0.2 mL, 1.57 mmol). The mixture was heated to 60° C. for 16 hours under nitrogen atmosphere. After cooling to room temperature, the mixture was concentrated in vacuo. The crude residue was purified by reverse phase chromatography (acetonitrile 8-38%/0.225% formic acid in water) to give the title compound (104 mg, 60%) as a brown solid. ¹H NMR (400 MHz, DMSO-d₆) δ 9.26 (s, 1H), 8.81-8.73 (m, 2H), 8.59 (d, J=5.6 Hz, 1H), 8.37-8.27 (m, 2H), 7.89 (d, J=5.6 Hz, 1H), 3.97-3.92 (m, 4H), 3.63 (s, 3H), 2.82 (t, J=6.8 Hz, 2H), 2.71-2.66 (m, 2H), 1.79-1.65 (m, 6H), 1.29 (s, 6H). LCMS (ESI) m/z: 447.1 [M+H]+.

### Step 2: 2-methyl-2-(8-(2-(pyridin-4-yl)pyrido[3,4-d]pyrimidin-4-yl)-2,8-diazaspiro[4.5]decan-2-yl)propan-1-ol formate

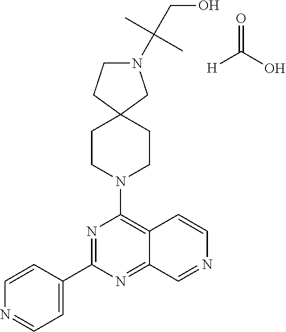

To a solution of methyl 2-methyl-2-(8-(2-(pyridin-4-yl)pyrido[3,4-d]pyrimidin-4-yl)-2,8-diazaspiro[4.5]decan-2-yl)propanoate (80 mg, 0.18 mmol) in THF (3 mL) was added lithium aluminum hydride (20 mg, 0.54 mmol) slowly at 0° C. The reaction mixture was stirred at 0° C. for 1 hour. The reaction was quenched with water (0.02 mL) and 15% aq. NaOH solution, diluted with EtOAc (20 mL), dried over anhydrous MgSO₄, filtered and concentrated in vacuo. The crude residue was purified by reverse phase chromatography (acetonitrile 5-35%/0.225% formic acid in water) to give the title compound (22 mg, 29%) as a yellow solid. ¹H NMR (400 MHz, DMSO-d₆) δ 9.25 (s, 1H), 8.76 (d, J=5.2 Hz, 2H), 8.58 (d, J=5.6 Hz, 1H), 8.33-8.29 (m, 3H), 7.87 (d, J=5.6 Hz, 1H), 4.07-3.83 (m, 4H), 3.36 (s, 2H), 3.01 (t, J=6.4 Hz, 2H), 2.87 (s, 2H), 1.85-1.65 (m, 6H), 1.08 (s, 6H). LCMS (ESI) m/z: 419.1 [M+H]+.

### Example 108

### 3-(8-(2-(pyridin-4-yl)pyrido[3,4-d]pyrimidin-4-yl)-2,8-diazaspiro[4.5]decan-2-yl)cyclobutanol (Compound 108) formate

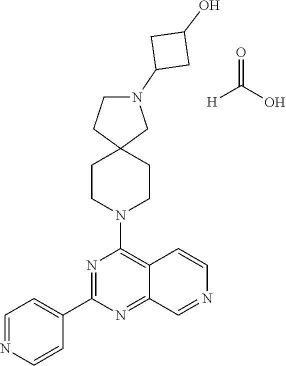

To a solution of 4-(2,8-diazaspiro[4.5]decan-8-yl)-2-(4-pyridyl)pyrido[3,4-d]pyrimidine hydrochloride (100 mg, 0.26 mmol) in 1,2-dichloroethane (1 mL) and MeOH (1 mL) was added N,N-diisopropylethylamine (0.14 mL, 0.78 mmol). The reaction mixture was stirred at room temperature for 5 min, acetic acid (0.07 mL, 1.31 mmol), 3-hydroxycyclobutanone (67 mg, 0.78 mmol) and sodium triacetoxyborohydride (166 mg, 0.78 mmol) were added to this mixture. The reaction mixture was heated to 60° C. for 16 hours. After cooling to room temperature, the mixture was filtrated and the filtrate was concentrated in vacuo. The crude residue was purified by reverse phase chromatography (acetonitrile 1-31%/0.225% formic acid in water) to give the title compound (15 mg, 14%) as a yellow solid and a mixture of diastereomers. ¹H NMR (400 MHz, DMSO-d₆) δ 9.26 (s, 1H), 8.80-8.73 (m, 2H), 8.59 (d, J=5.6 Hz, 1H), 8.35-8.30 (m, 2H), 8.18 (s, 1H), 7.89 (d, J=5.6 Hz, 1H), 4.96 (s, 1H), 4.28-4.17 (m, 1H), 4.05-3.96 (m, 2H), 3.94-3.86 (m, 2H), 3.83-3.73 (m, 1H), 2.92-2.84 (m, 1H), 2.55-2.51 (m, 1H), 2.41 (s, 2H), 2.32-2.11 (m, 2H), 1.93-1.65 (m, 8H). LCMS (ESI) m/z: 417.3 [M+H]⁺.

### Example 109

### 2-(8-(2-(pyridin-4-yl)pyrido[3,4-d]pyrimidin-4-yl)-2,8-diazaspiro[4.5]decan-2-yl)ethanesulfonamide (Compound 109)

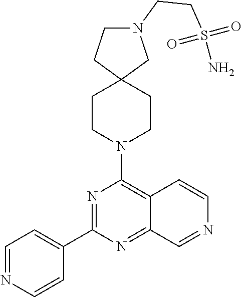

To a solution of 4-(2,8-diazaspiro[4.5]decan-8-yl)-2-(4-pyridyl)pyrido[3,4-d]pyrimidine hydrochloride (50 mg, 0.13 mmol) in MeOH (1 mL) was added N,N-diisopropylethylamine (0.11 mL, 0.65 mmol). The reaction mixture was stirred at room temperature for 5 minutes, and ethenesulfonamide (21 mg, 0.20 mmol) was added to this mixture. The reaction mixture was heated to 60° C. for 16 hours. After cooling to room temperature, the mixture was filtrated and the filtrate was concentrated in vacuo. The crude residue was purified by reverse phase chromatography (acetonitrile 25-55%/0.05% NH₃·H₂O+10 mM NH₄HCO₃ in water) to give the title compound (10 mg, 17%) as a yellow solid. ¹H NMR (400 MHz, DMSO-d₆) δ 9.26 (s, 1H), 8.80-8.74 (m, 2H), 8.59 (d, J=6.0 Hz, 1H), 8.35-8.30 (m, 2H), 7.89 (d, J=5.6 Hz, 1H), 6.79 (s, 2H), 4.05-3.85 (m, 4H), 3.32-3.27 (m, 2H), 3.20-3.12 (m, 2H), 2.85-2.74 (m, 2H), 2.61 (t, J=7.2 Hz, 2H), 1.81-1.67 (m, 6H). LCMS (ESI) m/z: 454.1 [M+H]+.

### Example 110

### 2-methyl-1-(8-(2-(pyridin-4-yl)pyrido[3,4-d]pyrimidin-4-yl)-2,8-diazaspiro[4.5]decan-2-yl)propan-2-ol (Compound 110)

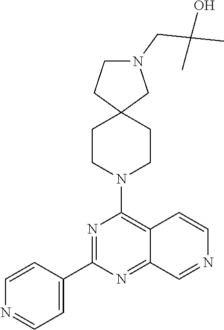 [SMILES: CC(C)(O)CN1CCC2(CCN(c3nc(-c4ccncc4)nc4cnccc34)CC2)C1]

To a solution of 4-(2,8-diazaspiro[4.5]decan-8-yl)-2-(4-pyridyl)pyrido[3,4-d]pyrimidine hydrochloride (50 mg, 0.13 mmol) in EtOH (1 mL) was added isobutyleneoxide (94 mg, 1.31 mmol) and K₂CO₃ (90 mg, 0.65 mmol). The reaction vessel was sealed and stirred at 110° C. under microwave for 30 minutes. After cooling to room temperature, the mixture was filtrated and the filtrate was concentrated in vacuo. The crude residue was purified by reverse phase chromatography (acetonitrile 37-67%/0.05% NH₃·H₂O+10 mM NH₄HCO₃ in water) to give the title compound (6.5 mg, 12%) as a yellow solid. ¹H NMR (400 MHz, DMSO-d₆) δ 9.24 (s, 1H), 8.76 (d, J=4.8 Hz, 2H), 8.57 (d, J=5.2 Hz, 1H), 8.34-8.27 (m, 2H), 7.90-7.86 (m, 1H), 4.07-4.03 (m, 1H), 4.02-3.95 (m, 2H), 3.91-3.81 (m, 2H), 2.69 (t, J=6.4 Hz, 2H), 2.57-2.55 (m, 2H), 2.36-2.31 (m, 2H), 1.83-1.68 (m, 4H), 1.65 (t, J=6.4 Hz, 2H), 1.10 (s, 6H). LCMS (ESI) m/z: 419.2 [M+H]+.

### Example 111

### 2-(pyridin-4-yl)-4-(2-(tetrahydrofuran-3-yl)-2,8-diazaspiro[4.5]decan-8-yl)pyrido[3,4-d]pyrimidine (Compound 111)

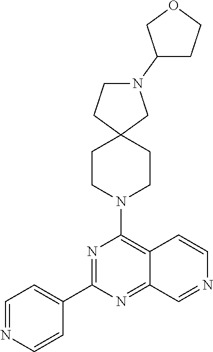 [SMILES: c1cc(-c2nc(N3CCC4(CC3)CCN(C3CCOC3)C4)c3ccncc3n2)ccn1]

Following the procedure described in Example 103 and making non-critical variations as required to replace hydroxyacetone with 3-oxotetrahydrofuran, the title compound was obtained as a white solid and a mixture of enantiomers. ¹H NMR (400 MHz, CD₃OD) δ 9.25 (s, 1H), 8.70 (d, J=5.2 Hz, 2H), 8.55 (d, J=5.6 Hz, 1H), 8.45 (d, J=5.6 Hz, 2H), 7.92 (d, J=6.0 Hz, 1H), 4.14-4.08 (m, 2H), 4.03-3.93 (m, 4H), 3.86-3.83 (m, 2H), 3.79-3.74 (m, 1H), 3.47-3.39 (m, 1H), 3.14-3.03 (m, 2H), 2.29-2.21 (m, 1H), 2.05-1.96 (m, 4H), 1.92-1.88 (m, 4H). LCMS (ESI) m/z: 417.1 [M+H]+.

### Examples 112 and 113

### (R)-2-(pyridin-4-yl)-4-(2-(tetrahydrofuran-3-yl)-2,8-diazaspiro[4.5]decan-8-yl)pyrido[3,4-d]pyrimidine and (S)-2-(pyridin-4-yl)-4-(2-(tetrahydrofuran-3-yl)-2,8-diazaspiro[4.5]decan-8-yl)pyrido[3,4-d]pyrimidine (Compounds 112 and 113)

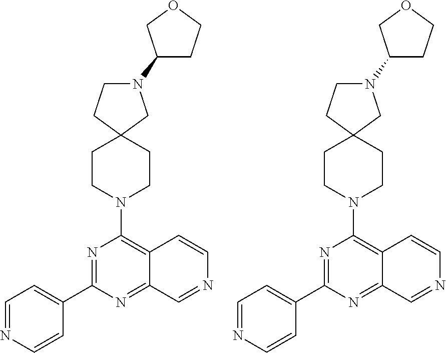

2-(pyridin-4-yl)-4-(2-(tetrahydrofuran-3-yl)-2,8-diazaspiro[4.5]decan-8-yl)pyrido[3,4-d]pyrimidine (80 mg, 0.19 mmol) was separated by using chiral SFC (Chiralpak IG (250 mm*30 mm, 10 um), Supercritical CO₂/EtOH+0.1% NH₄OH=40/60; 80 mL/min) to give the title compounds, both as white solids. Absolute configuration was arbitrarily assigned to each enantiomer. Example 112 (5 mg, second peak): ¹H NMR (400 MHz, DMSO-d₆) δ 9.26 (s, 1H), 8.77 (d, J=6.0 Hz, 2H), 8.59 (d, J=6.0 Hz, 1H), 8.32 (d, J=6.0 Hz, 2H), 7.89 (d, J=5.6 Hz, 1H), 4.04-3.96 (m, 2H), 3.91-3.84 (m, 2H), 3.79-3.70 (m, 2H), 3.69-3.61 (m, 1H), 3.53-3.46 (m, 1H), 2.88-2.74 (m, 1H), 2.63-2.54 (m, 2H), 2.02-1.87 (m, 2H), 1.83-1.60 (m, 8H). LCMS (ESI) m/z: 417.1 [M+H]+. Example 113 (10 mg, first peak): ¹H NMR (400 MHz, DMSO-d₆) δ 9.25 (s, 1H), 8.77 (d, J=5.2 Hz, 2H), 8.58 (d, J=5.6 Hz, 1H), 8.32 (d, J=5.2 Hz, 2H), 7.88 (d, J=5.2 Hz, 1H), 4.04-3.95 (m, 2H), 3.91-3.83 (m, 2H), 3.79-3.70 (m, 2H), 3.68-3.62 (m, 1H), 3.55-3.46 (m, 1H), 2.93-2.76 (m, 1H), 2.65-2.56 (m, 2H), 2.02-1.86 (m, 2H), 1.84-1.64 (m, 8H). LCMS (ESI) m/z: 417.1 [M+H]+.

### Example 114

### 2-(8-(2-(pyridin-4-yl)pyrido[3,4-d]pyrimidin-4-yl)-2,8-diazaspiro[4.5]decan-2-yl)ethanol (Compound 114) formate

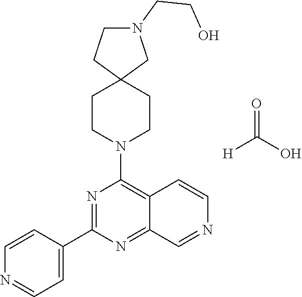 [SMILES: OCCN1CCC2(CCN(c3nc(-c4ccncc4)nc4cnccc34)CC2)C1 ; O=CO]

Following the procedure described in Example 107 and making non-critical variations as required to replace methyl 2-bromo-2-methylpropanoate with 2-bromoethanol, the title compound was obtained as a yellow solid. ¹H NMR (400 MHz, DMSO-d₆) δ 9.27-9.25 (m, 1H), 8.81-8.74 (m, 2H), 8.63-8.56 (m, 1H), 8.36-8.28 (m, 3H), 7.92-7.86 (m, 1H), 4.02-3.93 (m, 4H), 3.91-3.85 (m, 2H), 3.52-3.48 (m, 2H), 3.36-3.17 (m, 1H), 3.06 (s, 1H), 2.65-2.63 (m, 1H), 2.53 (s, 2H), 1.89 (t, J=6.8 Hz, 1H), 1.84-1.68 (m, 5H). LCMS (ESI) m/z: 391.1 [M+H]+.

### Examples 115 and 116

### (S)-1-(8-(2-(pyridin-4-yl)pyrido[3,4-d]pyrimidin-4-yl)-2,8-diazaspiro[4.5]decan-2-yl)propan-2-ol and (R)-1-(8-(2-(pyridin-4-yl)pyrido[3,4-d]pyrimidin-4-yl)-2,8-diazaspiro[4.5]decan-2-yl)propan-2-ol (Compounds 115 and 116)

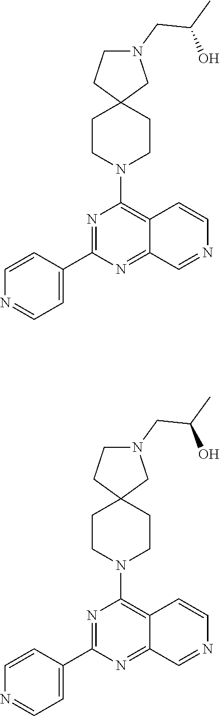 [SMILES: C[C@H](O)CN1CCC2(CCN(c3nc(-c4ccncc4)nc4cnccc34)CC2)C1 ; C[C@@H](O)CN1CCC2(CCN(c3nc(-c4ccncc4)nc4cnccc34)CC2)C1]

### Step 1: 1-(8-(2-(pyridin-4-yl)pyrido[3,4-d]pyrimidin-4-yl)-2,8-diazaspiro[4.5]decan-2-yl)propan-2-ol

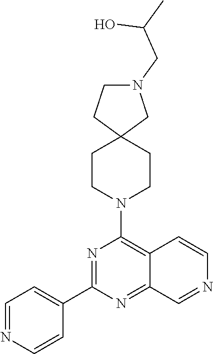 [SMILES: CC(O)CN1CCC2(CCN(c3nc(-c4ccncc4)nc4cnccc34)CC2)C1]

To a solution of 4-(2,8-diazaspiro[4.5]decan-8-yl)-2-(4-pyridyl)pyrido[3,4-d]pyrimidine hydrochloride (110 mg, 0.29 mmol) in EtOH (4 mL) was added 2-methyloxirane (80 mg, 1.38 mmol) and triethylamine (0.16 mL, 1.18 mmol). The mixture was stirred at room temperature for 16 hours. The reaction mixture was concentrated in vacuo. The crude residue was purified by reverse phase chromatography (acetonitrile 33-63%/0.05% NH₃·H₂O+10 mM NH₄HCO₃ in water) to give the title compound (40 mg, 34%) as a yellow solid. LCMS (ESI) m/z: 405.2 [M+H]+.

### Step 2: (S)-1-(8-(2-(pyridin-4-yl)pyrido[3,4-d]pyrimidin-4-yl)-2,8-diazaspiro[4.5]decan-2-yl)propan-2-ol and (R)-1-(8-(2-(pyridin-4-yl)pyrido[3,4-d]pyrimidin-4-yl)-2,8-diazaspiro[4.5]decan-2-yl)propan-2-ol

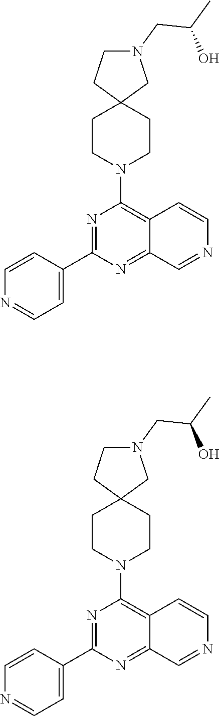 [SMILES: C[C@H](O)CN1CCC2(CCN(c3nc(-c4ccncc4)nc4cnccc34)CC2)C1 ; C[C@@H](O)CN1CCC2(CCN(c3nc(-c4ccncc4)nc4cnccc34)CC2)C1]

1-(8-(2-(pyridin-4-yl)pyrido[3,4-d]pyrimidin-4-yl)-2,8-diazaspiro[4.5]decan-2-yl)propan-2-ol (38 mg, 0.09 mmol) was separated by using chiral SFC (Chiralpak OJ (250 mm*30 mm, 10 um), Supercritical CO₂/EtOH+0.1% NH₄OH=60/40; 80 mL/min) to give the title compounds, both as yellow solids. Absolute configuration was arbitrarily assigned to each enantiomer. Example 115 (12 mg, second peak): ¹H NMR (400 MHz, DMSO-d₆) δ 9.24 (s, 1H), 8.76 (d, J=4.4 Hz, 2H), 8.58 (d, J=5.6 Hz, 1H), 8.31 (d, J=4.4 Hz, 2H), 7.87 (d, J=5.2 Hz, 1H), 4.03-3.93 (m, 2H), 3.92-3.80 (m, 2H), 3.79-3.62 (m, 2H), 2.71-2.61 (m, 2H), 2.54-2.47 (m, 2H), 2.42-2.32 (m, 2H), 1.82-1.72 (m, 4H), 1.71-1.64 (m, 2H), 1.06 (d, J=5.6 Hz, 2H). LCMS (ESI) m/z: 405.1 [M+H]+. Example 116 (12 mg, first peak): ¹H NMR (400 MHz, DMSO-d₆): δ 9.24 (s, 1H), 8.76 (d, J=4.4 Hz, 2H), 8.57 (d, J=5.6 Hz, 1H), 8.31 (d, J=4.4 Hz, 2H), 7.86 (d, J=5.6 Hz, 1H), 4.05-3.91 (m, 2H), 3.90-3.82 (m, 2H), 3.76-3.65 (m, 2H), 2.65-2.55 (m, 2H), 2.51-2.43 (m, 2H), 2.36-2.24 (m, 2H), 1.81-1.69 (m, 4H), 1.68-1.62 (m, 2H), 1.05 (d, J=5.6 Hz, 2H). LCMS (ESI) m/z: 405.1 [M+H]⁺.

### Example 117

### 4-(2-isopropyl-2,8-diazaspiro[4.5]decan-8-yl)-2-(pyridin-4-yl)pyrido[3,4-d]pyrimidine (Compound 117)

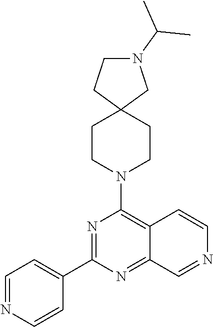 [SMILES: CC(C)N1CCC2(CCN(c3nc(-c4ccncc4)nc4cnccc34)CC2)C1]

Following the procedure described in Example 105 and making non-critical variations as required to replace 2,2,2-trifluoroethyl trifluoromethanesulfonate with 2-iodopropane, the title compound was obtained as a yellow solid. ¹H NMR (400 MHz, DMSO-d₆) δ 9.25 (s, 1H), 8.79-8.73 (m, 2H), 8.58 (d, J=5.6 Hz, 1H), 8.35-8.28 (m, 2H), 7.88 (d, J=5.6 Hz, 1H), 4.04-3.95 (m, 2H), 3.90-3.82 (m, 2H), 2.59 (t, J=6.8 Hz, 2H), 2.47 (s, 2H), 2.35-2.25 (m, 1H), 1.77-1.65 (m, 6H), 1.02 (d, J=6.4 Hz, 6H). LCMS (ESI) m/z: 389.2 [M+H]+.

### Example 118

### 3-(8-(2-(pyridin-4-yl)pyrido[3,4-d]pyrimidin-4-yl)-2,8-diazaspiro[4.5]decan-2-yl)cyclobutanecarbonitrile (Compound 118)

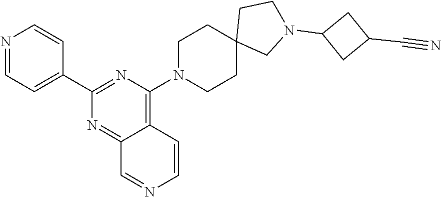 [SMILES: N#CC1CC(N2CCC3(CCN(c4nc(-c5ccncc5)nc5cnccc45)CC3)C2)C1]

Following the procedure described in Example 103 and making non-critical variations as required to replace hydroxyacetone with 3-oxocyclobutanecarbonitrile, the title compound was obtained as a mixture of diastereomers as a yellow solid. ¹H NMR (400 MHz, DMSO-d₆) δ 9.25 (s, 1H), 8.79-8.74 (m, 2H), 8.58 (d, J=5.6 Hz, 1H), 8.33-8.29 (m, 2H), 7.87 (d, J=5.6 Hz, 1H), 3.98-3.85 (m, 4H), 3.11-2.99 (m, 2H), 2.60-5.55 (m, 2H), 2.48-2.38 (m, 4H), 2.24-2.15 (m, 2H), 1.81-1.67 (m, 6H). LCMS (ESI) m/z: 448.1 [M+Na]⁺.

### Example 119

### 4-(2-cyclohexyl-2,8-diazaspiro[4.5]decan-8-yl)-2-(pyridin-4-yl)pyrido[3,4-d]pyrimidine (Compound 119) formate

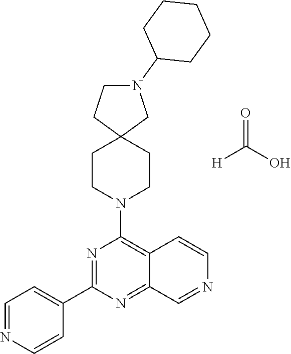 [SMILES: c1cc(-c2nc(N3CCC4(CC3)CCN(C3CCCCC3)C4)c3ccncc3n2)ccn1 ; O=CO]

Following the procedure described in Example 103 and making non-critical variations as required to replace hydroxyacetone with cyclohexanone, the title compound was obtained as a white solid. ¹H NMR (400 MHz, DMSO-d₆) δ 9.26 (s, 1H), 8.77 (d, J=4.8 Hz, 2H), 8.59 (d, J=5.6 Hz, 1H), 8.33 (d, J=4.4 Hz, 2H), 8.22 (s, 1H), 7.89 (d, J=5.6 Hz, 1H), 4.04-3.89 (m, 4H), 2.94-2.82 (m, 2H), 2.77-2.65 (m, 2H), 2.39-3.29 (m, 1H), 1.96-1.88 (m, 2H), 1.71-1.81 (m, 7H), 1.58-1.52 (m, 1H), 1.32-1.05 (m, 6H). LCMS (ESI) m/z: 429.1 [M+H]+.

### Example 120

### 3-(8-(2-(pyridin-4-yl)pyrido[3,4-d]pyrimidin-4-yl)-2,8-diazaspiro[4.5]decan-2-yl)propan-1-ol (Compound 120)

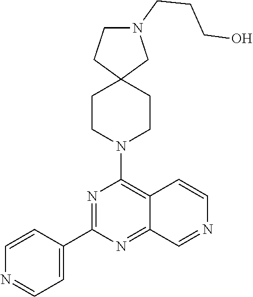 [SMILES: OCCCN1CCC2(CCN(c3nc(-c4ccncc4)nc4cnccc34)CC2)C1]

Following the procedure described in Example 107 and making non-critical variations as required to replace methyl 2-bromo-2-methylpropanoate with 3-bromo-1-propanol, the title compound was obtained as a white solid. ¹H NMR (400 MHz, DMSO-d₆) δ 9.27 (s, 1H), 8.80-8.74 (m, 2H), 8.60 (d, J=5.6 Hz, 1H), 8.35-8.31 (m, 2H), 7.89 (d, J=5.6 Hz, 1H), 4.04-3.97 (m, 2H), 3.93-3.85 (m, 2H), 3.46 (t, J=6.0 Hz, 2H), 2.78-2.50 (m, 6H), 1.81-1.71 (m, 6H), 1.67-1.59 (m, 2H). LCMS (ESI) m/z: 405.1 [M+H]+.

### Example 121

### 4-(2-(2-methoxyethyl)-2,8-diazaspiro[4.5]decan-8-yl)-2-(pyridin-4-yl)pyrido[3,4-d]pyrimidine (Compound 121)

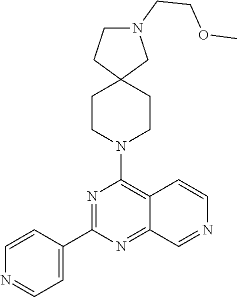 [SMILES: COCCN1CCC2(CCN(c3nc(-c4ccncc4)nc4cnccc34)CC2)C1]

Following the procedure described in Example 107 and making non-critical variations as required to replace methyl 2-bromo-2-methylpropanoate with 2-bromoethyl methyl ether, the title compound was obtained as a yellow solid. ¹H NMR (400 MHz, DMSO-d₆) δ 9.25 (s, 1H), 8.76 (d, J=5.2 Hz, 2H), 8.59 (d, J=5.6 Hz, 1H), 8.32 (d, J=5.2 Hz, 2H), 7.88 (d, J=5.6 Hz, 1H), 4.04-3.95 (m, 2H), 3.91-3.84 (m, 2H), 3.55-3.44 (m, 2H), 3.35 (s, 3H), 3.03-2.53 (m, 6H), 1.95-1.65 (m, 6H). LCMS (ESI) m/z: 405.1 [M+H]+.

### Example 122

### 2-(pyridin-4-yl)-4-(2-(tetrahydro-2H-pyran-4-yl)-2,8-diazaspiro[4.5]decan-8-yl)pyrido[3,4-d]pyrimidine (Compound 122) formate

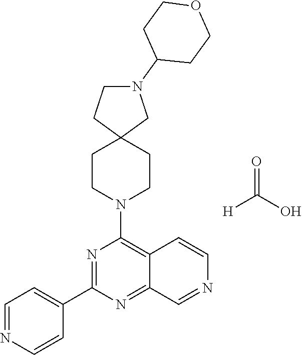 [SMILES: c1cc(-c2nc(N3CCC4(CC3)CCN(C3CCOCC3)C4)c3ccncc3n2)ccn1 ; O=CO]

Following the procedure described in Example 103 and making non-critical variations as required to replace hydroxyacetone with dihydro-2H-pyran-4 (3H)-one, the title compound was obtained as a white solid. ¹H NMR (400 MHz, DMSO-d₆) δ 9.26 (s, 1H), 8.77 (d, J=6.0 Hz, 2H), 8.59 (d, J=5.6 Hz, 1H), 8.33 (d, J=6.0 Hz, 2H), 8.18 (s, 1H), 7.89 (d, J=5.6 Hz, 1H), 4.05-3.97 (m, 2H), 3.92-3.83 (m, 4H), 3.42-3.25 (m, 2H), 2.73-2.66 (m, 2H), 2.58 (s, 2H), 2.34-2.26 (m, 1H), 1.82-1.68 (m, 8H), 1.46-1.35 (m, 2H). LCMS (ESI) m/z: 431.1 [M+H]+.

### Example 123

### N-(2-(8-(2-(pyridin-4-yl)pyrido[3,4-d]pyrimidin-4-yl)-2,8-diazaspiro[4.5]decan-2-yl)ethyl)acetamide (Compound 123)

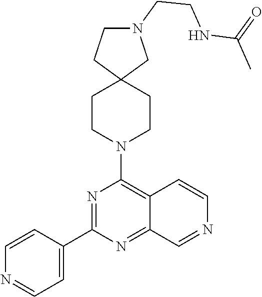 [SMILES: CC(=O)NCCN1CCC2(CCN(c3nc(-c4ccncc4)nc4cnccc34)CC2)C1]

### Step 1: tert-butyl (2-(8-(2-(pyridin-4-yl)pyrido[3,4-d]pyrimidin-4-yl)-2,8-diazaspiro[4.5]decan-2-yl)ethyl)carbamate

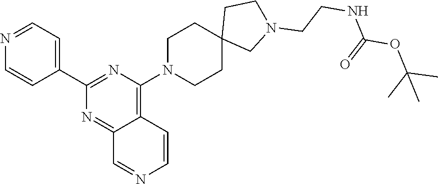 [SMILES: CC(C)(C)OC(=O)NCCN1CCC2(CCN(c3nc(-c4ccncc4)nc4cnccc34)CC2)C1]

Following the procedure described in Example 107 and making non-critical variations as required to replace methyl 2-bromo-2-methylpropanoate with tert-butyl (2-bromoethyl)carbamate, the title compound was obtained as a yellow solid. ¹H NMR (400 MHz, DMSO-d₆) δ 9.24 (s, 1H), 8.76 (d, J=5.6 Hz, 2H), 8.58 (d, J=5.6 Hz, 1H), 8.31 (d, J=5.6 Hz, 2H), 8.19 (s, 1H), 7.86 (d, J=5.6 Hz, 1H), 6.76 (t, J=5.6 Hz, 1H), 3.97-3.80 (m, 4H), 3.12-2.98 (m, 2H), 2.65 (t, J=6.8 Hz, 2H), 2.54 (s, 2H), 2.49-2.42 (m, 2H), 1.78-1.66 (m, 6H), 1.38 (s, 9H). LCMS (ESI) m/z: 490.1 [M+H]⁺.

### Step 2: 2-(8-(2-(pyridin-4-yl)pyrido[3,4-d]pyrimidin-4-yl)-2,8-diazaspiro[4.5]decan-2-yl)ethanamine hydrochloride

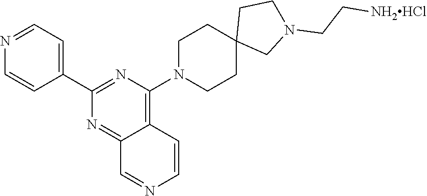

To a solution of tert-butyl (2-(8-(2-(pyridin-4-yl)pyrido[3,4-d]pyrimidin-4-yl)-2,8-diazaspiro[4.5]decan-2-yl)ethyl)carbamate (50 mg, 0.1 mmol) in EtOAc (0.6 mL) was added 4M HCl in EtOAc (0.6 mL, 2.2 mmol). The mixture was stirred at room temperature for 2 hours. The mixture was concentrated in vacuo to give the title compound (42 mg, crude) as a yellow solid that required no further purification. LCMS (ESI) m/z: 390.1 [M+H]⁺.

### Step 3: N-(2-(8-(2-(pyridin-4-yl)pyrido[3,4-d]pyrimidin-4-yl)-2,8-diazaspiro[4.5]decan-2-yl)ethyl)acetamide

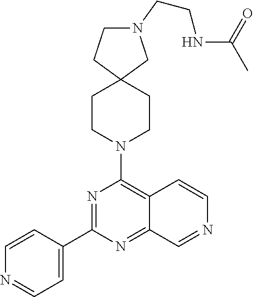 [SMILES: CC(=O)NCCN1CCC2(CCN(c3nc(-c4ccncc4)nc4cnccc34)CC2)C1]

To a solution of 2-(8-(2-(pyridin-4-yl)pyrido[3,4-d]pyrimidin-4-yl)-2,8-diazaspiro[4.5]decan-2-yl)ethanamine hydrochloride (15 mg, 0.04 mmol) and triethylamine (17 uL, 0.12 mmol) in DCM (0.5 mL) was added acetyl chloride (4 uL, 0.06 mmol) at 0° C. Then the reaction was warmed to room temperature and stirred for 2 hours. The reaction was quenched with sat. aq. NaHCO₃ (5 mL) and extracted with DCM (10 mL×2). The combined organic layers were dried over anhydrous Na₂SO₄, filtered and concentrated in vacuo. The crude residue was purified by reverse phase chromatography (acetonitrile 13-43%/0.05% NH₃·H₂O+10 mM NH₄HCO₃ in water) to give the title compound (7.6 mg, 49%) as a yellow solid. ¹H NMR (400 MHz, CD₃OD) δ 9.26 (s, 1H), 8.75-8.67 (m, 2H), 8.55 (d, J=5.6 Hz, 1H), 8.50-8.43 (m, 2H), 7.95 (d, J=5.6 Hz, 1H), 4.13-4.05 (m, 2H), 4.04-3.95 (m, 2H), 3.37-3.35 (m, 2H), 2.75 (t, J=6.8 Hz, 2H), 2.67-2.58 (m, 4H), 1.95 (s, 3H), 1.91-1.79 (m, 6H). LCMS (ESI) m/z: 432.1 [M+H].

### Example 124

### 4-(2-(oxetan-3-ylmethyl)-2,8-diazaspiro[4.5]decan-8-yl)-2-(pyridin-4-yl)pyrido[3,4-d]pyrimidine (Compound 124)

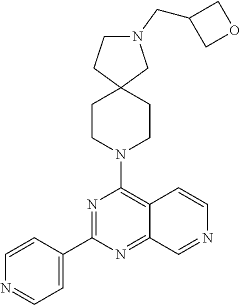 [SMILES: c1cc(-c2nc(N3CCC4(CCN(CC5COC5)C4)CC3)c3ccncc3n2)ccn1]

Following the procedure described in Example 103 and making non-critical variations as required to replace hydroxyacetone with oxetane-3-carbaldehyde, the title compound was obtained as a yellow solid. ¹H NMR (400 MHz, DMSO-d₆) δ 9.25 (s, 1H), 8.76 (d, J=5.6 Hz, 2H), 8.58 (d, J=5.6 Hz, 1H), 8.32 (d, J=5.6 Hz, 2H), 7.88 (d, J=6.0 Hz, 1H), 4.63-4.58 (m, 2H), 3.53-3.28 (m, 7H), 2.54-2.47 (m, 4H), 1.88-1.84 (m, 2H), 2.42 (s, 2H), 1.40-1.31 (m, 6H). LCMS (ESI) m/z: 417.1 [M+H]⁺.

### Example 125

### 1-(8-(2-(pyridin-4-yl)pyrido[3,4-d]pyrimidin-4-yl)-2,8-diazaspiro[4.5]decan-2-yl)ethan-1-one (Compound 125)

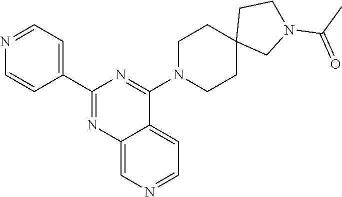 [SMILES: CC(=O)N1CCC2(CCN(c3nc(-c4ccncc4)nc4cnccc34)CC2)C1]

Following the procedure described in Example 101, Step 3 and making non-critical variations as required to replace tert-butyl 2,8-diazaspiro[4.5]decane-2-carboxylate with 1-(2,8-diazaspiro[4.5]decan-2-yl)ethan-1-one the title compound was obtained. LCMS (ESI) m/z: 389.2 [M+H]⁺. ¹H NMR (400 MHz, DMSO-d₆) δ 9.28 (d, J=2.5, 1H), 8.83-8.73 (m, 2H), 8.64-8.58 (m, 1H), 8.37-8.28 (m, 2H), 7.91 (d, J=5.8, 1H), 4.12-3.86 (m, 4H), 3.67-3.58 (m, 1H), 3.57-3.51 (m, 1H), 3.42-3.36 (m, 1H), 3.19-3.09 (m, 1H), 1.99-1.93 (m, 3H), 1.94-1.90 (m, 1H), 1.86-1.81 (m, 1H), 1.80-1.72 (m, 4H).

### Example 126

### N-(2-(8-(2-(pyridin-4-yl)pyrido[3,4-d]pyrimidin-4-yl)-2,8-diazaspiro[4.5]decan-2-yl)ethyl)methanesulfonamide (Compound 126)

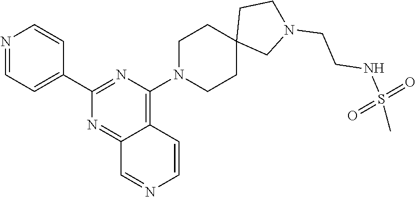 [SMILES: CS(=O)(=O)NCCN1CCC2(CCN(c3nc(-c4ccncc4)nc4cnccc34)CC2)C1]

To a solution of 2-(8-(2-(pyridin-4-yl)pyrido[3,4-d]pyrimidin-4-yl)-2,8-diazaspiro[4.5]decan-2-yl)ethanamine hydrochloride (42 mg, 0.1 mmol), 4-dimethylaminopyridine (2.4 mg, 0.02 mmol) and triethylamine (43 uL, 0.3 mmol) in DCM (1 mL) was added methanesulfonyl chloride (10 uL, 0.11 mmol) at 0° C. Then the reaction was warmed to room temperature and stirred for 4 hours. The reaction was quenched with sat. aq. NaHCO₃ (10 mL) and extracted with DCM (20 mL×2). The combined organic layers were dried over anhydrous Na₂SO₄, filtered and concentrated in vacuo. The mixture was concentrated in vacuo. The crude residue was purified by reverse phase chromatography (acetonitrile 25-55%/0.05% NH₃·H₂O+10 mM NH₄HCO₃ in water) to give the title compound (7.6 mg, 16%) as a yellow solid. ¹H NMR (400 MHz, DMSO-d₆) δ 9.25 (s, 1H), 8.83-8.70 (m, 2H), 8.58 (d, J=5.6 Hz, 1H), 8.36-8.24 (m, 2H), 7.88 (d, J=5.6 Hz, 1H), 6.93 (s, 1H), 4.02-3.84 (m, 4H), 3.05 (t, J=6.8 Hz, 2H), 2.93 (s, 3H), 2.59 (t, J=6.8 Hz, 2H), 2.54-2.50 (m, 2H), 2.48 (s, 2H), 1.81-1.65 (m, 6H). LCMS (ESI) m/z: 468.1 [M+H]⁺.

### Example 127

### 4-(2-(2,2-difluoroethyl)-2,8-diazaspiro[4.5]decan-8-yl)-2-(pyridin-4-yl)pyrido[3,4-d]pyrimidine (Compound 127)

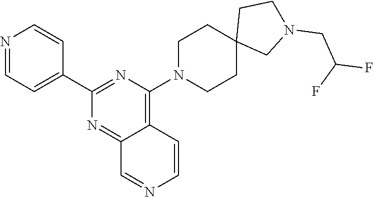 [SMILES: FC(F)CN1CCC2(CCN(c3nc(-c4ccncc4)nc4cnccc34)CC2)C1]

To a solution of 4-(2,8-diazaspiro[4.5]decan-8-yl)-2-(4-pyridyl)pyrido[3,4-d]pyrimidine hydrochloride (30 mg, 0.09 mmol) in acetonitrile (1.5 mL) was added triethylamine (0.04 mL, 0.26 mmol) and 2,2-difluoroethyl trifluoromethanesulfonate (28 mg, 0.13 mmol). The mixture was heated to 50° C. for 16 hours under nitrogen atmosphere. After cooling to room temperature, the mixture was concentrated in vacuo, the resulting residue was purified by reverse phase chromatography (acetonitrile 2-32%/0.225% formic acid in water) to give the title compound (12 mg, 32%) as a yellow solid. ¹H NMR (400 MHz, DMSO-d₆) δ 9.27 (s, 1H), 8.82 (d, J=6.0 Hz, 2H), 8.78 (d, J=6.0 Hz, 1H), 8.33 (d, J=6.0 Hz, 2H), 7.89 (d, J=6.0 Hz, 1H), 6.47-6.09 (m, 1H), 4.05-3.97 (m, 2H), 3.92-3.83 (m, 2H), 3.30-2.65 (m, 6H), 1.90-1.73 (m, 6H). LCMS (ESI) m/z: 411.3 [M+H]⁺.

### Examples 128 and 129

### (1R,2R)-2-(8-(2-(pyridin-4-yl)pyrido[3,4-d]pyrimidin-4-yl)-2,8-diazaspiro[4.5]decan-2-yl)cyclopentanol and (1S,2S)-2-(8-(2-(pyridin-4-yl)pyrido[3,4-d]pyrimidin-4-yl)-2,8-diazaspiro[4.5]decan-2-yl)cyclopentanol (Compounds 128 and 129)

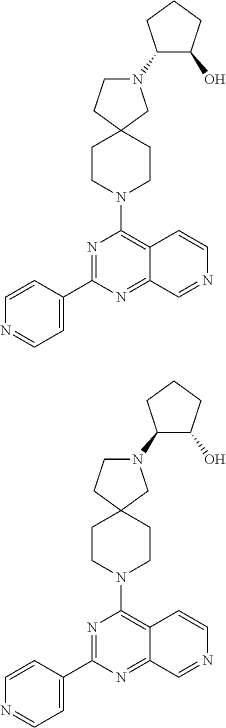 [SMILES: O[C@@H]1CCC[C@H]1N1CCC2(CCN(c3nc(-c4ccncc4)nc4cnccc34)CC2)C1 ; O[C@H]1CCC[C@@H]1N1CCC2(CCN(c3nc(-c4ccncc4)nc4cnccc34)CC2)C1]

### Step 1: trans-2-(8-(2-(pyridin-4-yl)pyrido[3,4-d]pyrimidin-4-yl)-2,8-diazaspiro[4.5]decan-2-yl)cyclopentanol

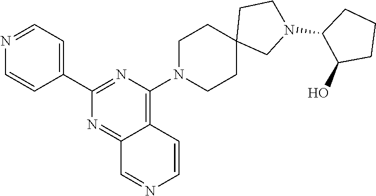 [SMILES: O[C@@H]1CCC[C@H]1N1CCC2(CCN(c3nc(-c4ccncc4)nc4cnccc34)CC2)C1]

To a solution of 4-(2,8-diazaspiro[4.5]decan-8-yl)-2-(4-pyridyl)pyrido[3,4-d]pyrimidine hydrochloride (200 mg, 0.52 mmol) in EtOH (5 mL) was added 1,2-epoxycyclopentane (0.46 mL, 5.22 mmol) and K₂CO₃ (361 mg, 2.61 mmol). The reaction mixture was heated to 80° C. for 16 hours. After cooling to room temperature, the reaction mixture was diluted with DCM (100 mL), washed with water (30 mL) and brine (30 mL). The organic layer was dried over anhydrous Na₂SO₄, filtered and concentrated in vacuo. The crude residue was purified by reverse phase chromatography (acetonitrile 25-55%/0.05% NH₃·H₂O+10 mM NH₄HCO₃ in water) to give the title compound (83 mg, 37%) as a yellow solid. LCMS (ESI) m/z: 431.2 [M+H]⁺.

### Step 2: (1R,2R)-2-(8-(2-(pyridin-4-yl)pyrido[3,4-d]pyrimidin-4-yl)-2,8-diazaspiro[4.5]decan-2-yl)cyclopentanol and (1S,2S)-2-(8-(2-(pyridin-4-yl)pyrido[3,4-d]pyrimidin-4-yl)-2,8-diazaspiro[4.5]decan-2-yl)cyclopentanol

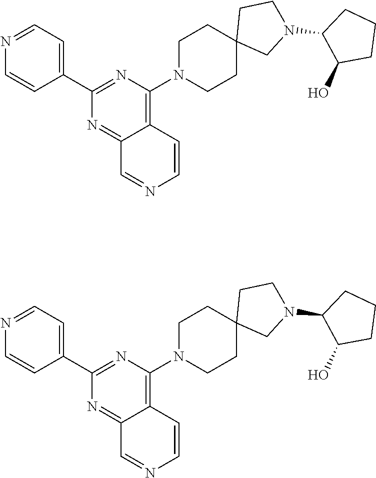 [SMILES: O[C@@H]1CCC[C@H]1N1CCC2(CCN(c3nc(-c4ccncc4)nc4cnccc34)CC2)C1 ; O[C@H]1CCC[C@@H]1N1CCC2(CCN(c3nc(-c4ccncc4)nc4cnccc34)CC2)C1]

trans-2-(8-(2-(pyridin-4-yl)pyrido[3,4-d]pyrimidin-4-yl)-2,8-diazaspiro[4.5]decan-2-yl)cyclopentanol (30 mg, 0.07 mmol) was separated by using chiral SFC (Phenomenex-Chiralpak-IG (250 mm*30 mm, 10 um), Supercritical CO₂/i-PrOH+0.1% NH₄OH=60/40; 80 mL/min) to give the title compounds, both as white solids. Absolute configuration was arbitrarily assigned to each enantiomer. Example 128 (8.1 mg, first peak): ¹H NMR (400 MHz, DMSO-d₆) δ 9.25 (s, 1H), 8.76 (d, J=5.6 Hz, 2H), 8.58 (d, J=5.6 Hz, 1H), 8.31 (d, J=5.6 Hz, 2H), 7.88 (d, J=5.6 Hz, 1H), 4.56-4.42 (s, 1H), 4.04-3.95 (m, 2H), 3.93-3.80 (m, 3H), 2.64-2.57 (m, 2H), 2.47-2.44 (m, 1H), 2.35-2.25 (m, 1H), 1.82-1.67 (m, 6H), 1.64 (t, J=6.8 Hz, 2H), 1.59-1.50 (m, 2H), 1.48-1.36 (m, 2H). LCMS (ESI) m/z: 431.2 [M+H]⁺. Example 129 (12.6 mg, second peak): ¹H NMR (400 MHz, DMSO-d₆) δ 9.25 (s, 1H), 8.76 (d, J=5.6 Hz, 2H), 8.58 (d, J=5.6 Hz, 1H), 8.31 (d, J=5.6 Hz, 2H), 7.88 (d, J=5.6 Hz, 1H), 4.56-4.42 (s, 1H), 4.04-3.95 (m, 2H), 3.93-3.80 (m, 3H), 2.64-2.57 (m, 2H), 2.47-2.44 (m, 1H), 2.35-2.25 (m, 1H), 1.82-1.67 (m, 6H), 1.64 (t, J=6.8 Hz, 2H), 1.59-1.50 (m, 2H), 1.48-1.36 (m, 2H). LCMS (ESI) m/z: 431.2 [M+H]⁺.

### Example 130

### 2-(8-(2-(pyridin-4-yl)pyrido[3,4-d]pyrimidin-4-yl)-2,8-diazaspiro[4.5]decan-2-yl)acetamide (Compound 130) formate

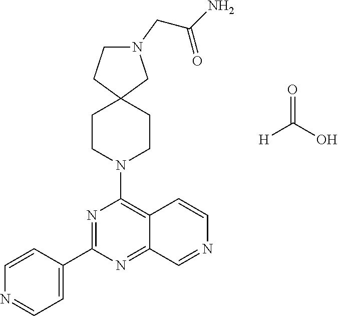 [SMILES: NC(=O)CN1CCC2(CCN(c3nc(-c4ccncc4)nc4cnccc34)CC2)C1 ; O=CO]

Following the procedure described in Example 106 and making non-critical variations as required to replace methyl cyclopentyl bromide with 2-bromoacetamide, the title compound was obtained as a white solid. ¹H NMR (400 MHz, CD₃OD) δ 9.24 (s, 1H), 8.70 (d, J=5.6 Hz, 2H), 8.54 (d, J=5.6 Hz, 1H), 8.44 (d, J=4.8 Hz, 2H), 8.36 (s, 1H), 7.91 (d, J=5.6 Hz, 1H), 4.12-4.05 (m, 2H), 4.02-3.95 (m, 2H), 3.64 (s, 2H), 3.20 (t, J=7.2 Hz, 2H), 3.06 (s, 2H), 2.02 (t, J=7.2 Hz, 2H), 1.97-1.90 (m, 4H). LCMS (ESI) m/z: 404.1 [M+H]⁺.

### Example 131

### 4-(2-cyclobutyl-2,8-diazaspiro[4.5]decan-8-yl)-2-(pyridin-4-yl)pyrido[3,4-d]pyrimidine (Compound 131)

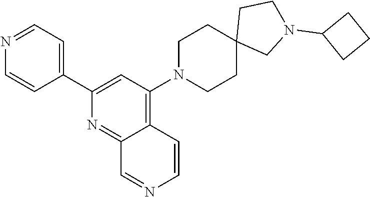 [SMILES: c1cc(-c2cc(N3CCC4(CC3)CCN(C3CCC3)C4)c3ccncc3n2)ccn1]

Following the procedure described in Example 103 and making non-critical variations as required to replace hydroxyacetone with cyclobutanone, the title compound was obtained as a yellow solid. ¹H NMR (400 MHz, DMSO-d₆) δ 9.26 (s, 1H), 8.79-8.75 (m, 2H), 8.59 (d, J=5.6 Hz, 1H), 8.35-8.31 (m, 2H), 7.89 (d, J=6.0 Hz, 1H), 4.02-3.95 (m, 2H), 3.93-3.86 (m, 2H), 2.89 (m, 1H), 2.49-2.45 (m, 2H), 2.37 (s, 2H), 1.97-1.83 (m, 4H), 1.82-1.60 (m, 8H). LCMS (ESI) m/z: 401.2 [M+H]⁺.

### Example 132

### 3-(8-(2-(pyridin-4-yl)pyrido[3,4-d]pyrimidin-4-yl)-2,8-diazaspiro[4.5]decan-2-yl)propanamide (Compound 132)

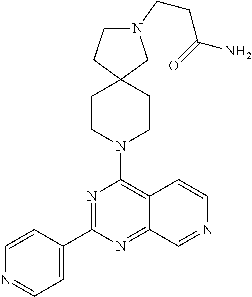 [SMILES: NC(=O)CCN1CCC2(CCN(c3nc(-c4ccncc4)nc4cnccc34)CC2)C1]

Following the procedure described in Example 109 and making non-critical variations as required to replace ethenesulfonamide with acrylamide, the title compound was obtained as a white solid. ¹H NMR (400 MHz, DMSO-d₆) δ 9.22 (s, 1H), 8.76-8.73 (m, 2H), 8.56 (d, J=5.6 Hz, 1H), 8.32-8.26 (m, 2H), 7.84 (s, 1H), 7.40 (s, 1H), 6.78 (s, 1H), 3.99-3.90 (m, 2H), 3.89-3.80 (m, 2H), 2.60-2.53 (m, 4H), 2.43 (s, 2H), 2.22 (t, J=7.2 Hz, 2H), 1.78-1.62 (m, 6H). LCMS (ESI) m/z: 418.1 [M+H]⁺.

### Example 133

### 4-(2-benzyl-2,8-diazaspiro[4.5]decan-8-yl)-2-(pyridin-4-yl)pyrido[3,4-d]pyrimidine (Compound 133)

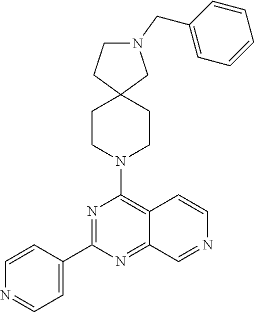 [SMILES: c1ccc(CN2CCC3(CCN(c4nc(-c5ccncc5)nc5cnccc45)CC3)C2)cc1]

Following the procedure described in Example 103 and making non-critical variations as required to replace hydroxyacetone with benzaldehyde, the title compound was obtained as a yellow solid. ¹H NMR (400 MHz, DMSO-d₆) δ 9.25 (s, 1H), 8.79-8.74 (m, 2H), 8.58 (d, J=6.0 Hz, 1H), 8.33-8.29 (m, 2H), 7.87 (d, J=6.0 Hz, 1H), 7.40-7.25 (m, 5H), 4.03-3.94 (m, 2H), 3.90-3.81 (m, 2H), 3.68 (s, 2H), 3.34-3.26 (m, 2H), 2.67 (s, 2H), 1.86-1.70 (m, 6H). LCMS (ESI) m/z: 437.1 [M+H]⁺.

### Example 134

### 4-(2-(2-fluoroethyl)-2,8-diazaspiro[4.5]decan-8-yl)-2-(pyridin-4-yl)pyrido[3,4-d]pyrimidine (Compound 134) formate

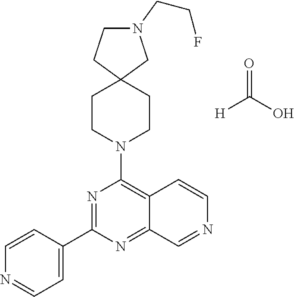 [SMILES: FCCN1CCC2(CCN(c3nc(-c4ccncc4)nc4cnccc34)CC2)C1 ; O=CO]

Following the procedure described in Example 106 and making non-critical variations as required to replace methyl cyclopentyl bromide with 1-bromo-2-fluoroethane, the title compound was obtained as a yellow solid. ¹H NMR (400 MHz, DMSO-d₆) δ 9.25 (s, 1H), 8.78-8.75 (m, 2H), 8.58 (d, J=5.6 Hz, 1H), 8.35-8.29 (m, 2H), 8.18 (s, 1H), 7.88 (d, J=5.6 Hz, 1H), 4.60-4.45 (m, 2H), 4.03-3.96 (m, 2H), 3.91-3.84 (m, 2H), 2.77-2.66 (m, 2H), 2.64 (t, J=6.8 Hz, 2H), 2.52 (s, 2H), 1.81-1.72 (m, 4H), 1.72-1.68 (m, 2H). LCMS (ESI) m/z: 393.1 [M+H]⁺.

### Example 135

### 4-(2-(3-fluoropropyl)-2,8-diazaspiro[4.5]decan-8-yl)-2-(pyridin-4-yl)pyrido[3,4-d]pyrimidine (Compound 135) formate

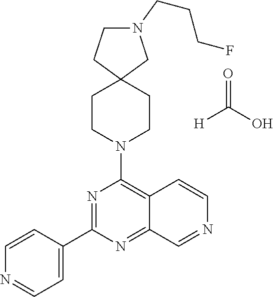

Following the procedure described in Example 107 and making non-critical variations as required to replace methyl 2-bromo-2-methylpropanoate with 1-bromo-3-fluoropropane, the title compound was obtained as a light yellow solid. ¹H NMR (400 MHz, DMSO-d₆) δ 9.24 (s, 1H), 8.76 (d, J=5.6 Hz, 2H), 8.58 (d, J=5.6 Hz, 1H), 8.31 (d, J=6.0 Hz, 2H), 8.20 (s, 1H), 7.87 (d, J=5.6 Hz, 1H), 4.57-4.41 (m, 2H), 4.01-3.94 (m, 2H), 3.91-3.85 (m, 2H), 2.66 (t, J=6.8 Hz, 2H), 2.58-2.53 (m, 4H), 1.91-1.76 (m, 4H), 1.73-1.69 (m, 4H). LCMS (ESI) m/z: 407.1 [M+H]⁺.

### Example 136

### 3-(8-(2-(pyridin-4-yl)pyrido[3,4-d]pyrimidin-4-yl)-2,8-diazaspiro[4.5]decan-2-yl)propanenitrile (Compound 136)

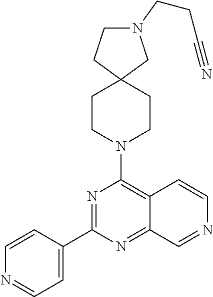 [SMILES: N#CCCN1CCC2(CCN(c3nc(-c4ccncc4)nc4cnccc34)CC2)C1]

Following the procedure described in Example 107 and making non-critical variations as required to replace methyl 2-bromo-2-methylpropanoate with 3-bromopropanenitrile, the title compound was obtained as a yellow solid. ¹H NMR (400 MHz, DMSO-d₆) δ 9.26 (s, 1H), 8.77 (d, J=6.0 Hz, 2H), 8.59 (d, J=5.6 Hz, 1H), 8.33 (d, J=6.0 Hz, 2H), 7.89 (d, J=5.6 Hz, 1H), 4.03-3.95 (m, 2H), 3.92-3.84 (m, 2H), 2.71-2.58 (m, 6H), 2.52-2.51 (m, 2H), 1.80-1.67 (m, 6H). LCMS (ESI) m/z: 400.1 [M+H]⁺.

### Example 137

### 4-(2-(oxetan-3-yl)-2,8-diazaspiro[4.5]decan-8-yl)-2-(pyridin-4-yl)pyrido[3,4-d]pyrimidine (Compound 137) formate

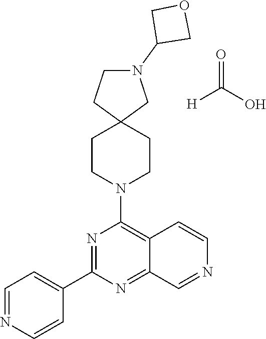

Following the procedure described in Example 103 and making non-critical variations as required to replace hydroxyacetone with oxetan-3-one, the title compound was obtained as a yellow solid. ¹H NMR (400 MHz, DMSO-d₆) δ 9.26 (s, 1H), 8.77 (d, J=5.6 Hz, 2H), 8.59 (d, J=5.6 Hz, 1H), 8.33 (d, J=6.0 Hz, 2H), 8.15 (s, 1H), 7.89 (d, J=6.0 Hz, 1H), 4.59-4.54 (m, 2H), 4.49-4.44 (m, 2H), 4.03-3.86 (m, 4H), 3.62-3.54 (m, 1H), 2.58-2.55 (m, 2H), 2.39 (s, 2H), 1.83-1.68 (m, 6H). LCMS (ESI) m/z: 403.1 [M+H]⁺.

### Example 138

### 4-(2-ethyl-2,8-diazaspiro[4.5]decan-8-yl)-2-(pyridin-4-yl)pyrido[3,4-d]pyrimidine (Compound 138)

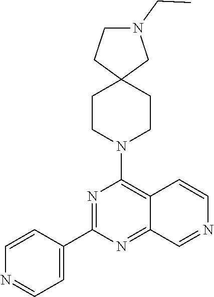 [SMILES: CCN1CCC2(CCN(c3nc(-c4ccncc4)nc4cnccc34)CC2)C1]

To a solution of 4-(2,8-diazaspiro[4.5]decan-8-yl)-2-(4-pyridyl)pyrido[3,4-d]pyrimidine hydrochloride (50 mg, 0.13 mmol) in acetonitrile (2 mL) was added K₂CO₃ (22 mg, 0.16 mmol) and iodoethane (0.01 mL, 0.12 mmol). The mixture was stirred at room temperature for 16 hours. The mixture was filtrated and the filtrate was concentrated in vacuo. The crude residue was purified by reverse phase chromatography (acetonitrile 35-65%/0.05% NH₃·H₂O+10 mM NH₄HCO₃ in water) to give the title compound (8.3 mg, 12%) as a white solid. ¹H NMR (400 MHz, DMSO-d₆) δ 9.27 (s, 1H), 8.82-8.73 (m, 2H), 8.60 (d, J=6.0 Hz, 1H), 8.36-8.30 (m, 2H), 7.90 (d, J=6.0 Hz, 1H), 4.06-3.96 (m, 2H), 3.95-3.85 (m, 2H), 3.31-3.24 (m, 2H), 3.10-2.70 (m, 4H), 1.89-1.73 (m, 6H), 1.21-1.06 (m, 3H). LCMS (ESI) m/z: 375.1 [M+H]⁺.

### Example 139

### 4-(2-(1H-pyrazol-3-yl)-2,8-diazaspiro[4.5]decan-8-yl)-2-(pyridin-4-yl)pyrido[3,4-d]pyrimidine (Compound 139)

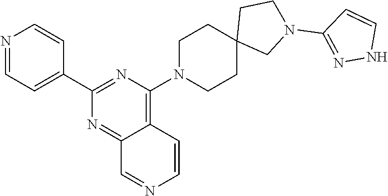 [SMILES: c1cc(-c2nc(N3CCC4(CCN(c5cc[nH]n5)C4)CC3)c3ccncc3n2)ccn1]

### Step 1: 2-(pyridin-4-yl)-4-(2-(1-((2-(trimethylsilyl)ethoxy)methyl)-1H-pyrazol-3-yl)-2,8-diazaspiro[4.5]decan-8-yl)pyrido[3,4-d]pyrimidine

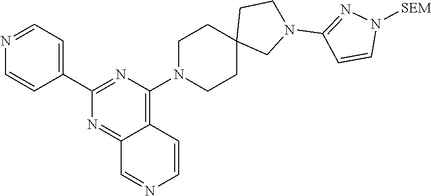 [SMILES: C[Si](C)(C)CCOCn1ccc(N2CCC3(CCN(c4nc(-c5ccncc5)nc5cnccc45)CC3)C2)n1]

To a solution of 4-(2,8-diazaspiro[4.5]decan-8-yl)-2-(4-pyridyl)pyrido[3,4-d]pyrimidine hydrochloride (200 mg, 0.52 mmol), 3-bromo-1-((2-(trimethylsilyl)ethoxy)methyl)-1H-pyrazole (217 mg, 0.78 mmol) (prepared according to the procedure in WO201934890) and sodium tert-butoxide (251 mg, 2.61 mmol) in 2-methyl-2-butanol (5 mL) was added allylpalladium(II) chloride dimer (19 mg, 0.05 mmol) and 2-(di-tert-butylphosphino)-2′,4′,6′-triisopropyl-3,6-dimethoxy-1,1′-biphenyl (51 mg, 0.1 mmol). The reaction mixture was heated to 90° C. under nitrogen atmosphere for 16 hours. After cooling to room temperature, the solvent was removed in vacuo, the residue was purified by silica gel chromatography (solvent gradient: 0-5% MeOH in DCM) to give the title compound (100 mg, 35%) as yellow oil. LCMS (ESI) m/z: 543.3 [M+H]⁺.

### Step 2: 4-(2-(1H-pyrazol-3-yl)-2,8-diazaspiro[4.5]decan-8-yl)-2-(pyridin-4-yl)pyrido[3,4-d]pyrimidine

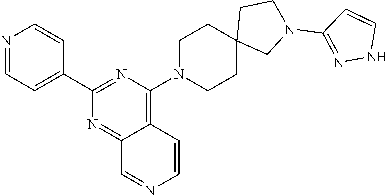 [SMILES: c1cc(-c2nc(N3CCC4(CCN(c5cc[nH]n5)C4)CC3)c3ccncc3n2)ccn1]

To a solution of 2-(pyridin-4-yl)-4-(2-(1-((2-(trimethylsilyl)ethoxy)methyl)-1H-pyrazol-3-yl)-2,8-diazaspiro[4.5]decan-8-yl)pyrido[3,4-d]pyrimidine (100 mg, 0.18 mmol) in DCM (2 mL) was added trifluoroacetic acid (0.53 mL, 7.13 mmol). The mixture was stirred at room temperature for 2 hours. The mixture was concentrated in vacuo, the residue was diluted in MeOH (2 mL), and the pH was adjusted to 8 by addition of ammonium hydroxide (30% in water). The crude mixture was purified by reverse phase chromatography (acetonitrile 25-55%/0.05% NH₃·H₂O+10 mM NH₄HCO₃ in water) to give the title compound (6 mg, 8%) as a white solid. ¹H NMR (400 MHz, DMSO-d₆) δ 11.63 (s, 1H), 9.27 (s, 1H), 8.80-8.74 (m, 2H), 8.60 (d, J=5.6 Hz, 1H), 8.36-8.30 (m, 2H), 7.92 (d, J=6.0 Hz, 1H), 7.44 (s, 1H), 5.53 (s, 1H), 4.09-4.01 (m, 2H), 4.01-3.93 (m, 2H), 3.30-3.28 (m, 2H), 3.19 (s, 2H), 1.91 (t, J=6.4 Hz, 2H), 1.84-1.77 (m, 4H). LCMS (ESI) m/z: 413.1 [M+H]⁺.

### Example 140

### 4-(2-phenyl-2,8-diazaspiro[4.5]decan-8-yl)-2-(pyridin-4-yl)pyrido[3,4-d]pyrimidine (Compound 140)

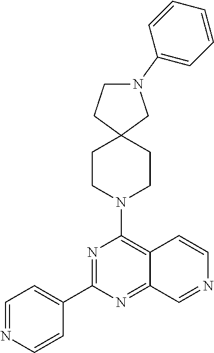 [SMILES: c1ccc(N2CCC3(CCN(c4nc(-c5ccncc5)nc5cnccc45)CC3)C2)cc1]

To a solution of 4-(2,8-diazaspiro[4.5]decan-8-yl)-2-(4-pyridyl)pyrido[3,4-d]pyrimidine hydrochloride (100 mg, 0.26 mmol), iodobenzene (0.04 mL, 0.35 mmol) and Cs₂CO₃ (280 mg, 0.87 mmol) in 1,4-dioxane (2 mL) was added palladium(II)acetate (13 mg, 0.06 mmol) and (±)-2,2′-bis(diphenylphosphino)-1,1′-binaphthalene (36 mg, 0.06 mmol). The reaction mixture was heated to 110° C. under nitrogen atmosphere for 16 hours. After cooling to room temperature, the mixture was filtrated and the filtrate was concentrated in vacuo. The crude residue was purified by reverse phase chromatography (acetonitrile 20-50%/0.05% NH₃·H₂O+10 mM NH₄HCO₃ in water) to give the title compound (9 mg, 7%) as a white solid. ¹H NMR (400 MHz, DMSO-d₆) δ 9.27 (s, 1H), 8.75 (d, J=4.0 Hz, 2H), 8.58 (d, J=5.2 Hz, 1H), 8.34 (d, J=4.0 Hz, 2H), 7.92 (d, J=5.2 Hz, 1H), 7.19-7.12 (m, 2H), 6.62-6.31 (m, 3H), 4.12-3.91 (m, 4H), 3.34-3.30 (m, 2H), 3.24 (s, 2H), 2.12-1.89 (m, 2H), 1.88-1.72 (m, 4H). LCMS (ESI) m/z: 423.2 [M+H]⁺.

### Example 141

### 4-(2-(1H-pyrazol-4-yl)-2,8-diazaspiro[4.5]decan-8-yl)-2-(pyridin-4-yl)pyrido[3,4-d]pyrimidine (Compound 141) trifluoroacetate

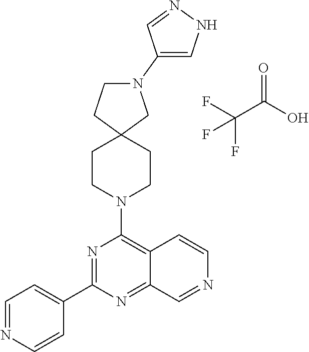 [SMILES: c1cc(-c2nc(N3CCC4(CCN(c5cn[nH]c5)C4)CC3)c3ccncc3n2)ccn1 ; O=C(O)C(F)(F)F]

Following the procedure described in Example 139 and making non-critical variations as required to replace 3-bromo-1-((2-(trimethylsilyl)ethoxy)methyl)-1H-pyrazole with 4-iodo-1-((2-(trimethylsilyl)ethoxy)methyl)-1H-pyrazole, the title compound was obtained as a yellow solid. ¹H NMR (400 MHz, DMSO-d₆) δ 9.26 (s, 1H), 8.77 (d, J=5.2 Hz, 2H), 8.59 (d, J=5.6 Hz, 1H), 8.33 (d, J=5.6 Hz, 2H), 8.23 (s, 1H), 7.90 (d, J=5.6 Hz, 1H), 7.09 (s, 2H), 4.06-3.92 (m, 4H), 3.10 (t, J=6.8 Hz, 2H), 2.99 (s, 2H), 1.90-1.86 (m, 2H), 1.85-1.72 (m, 4H). LCMS (ESI) m/z: 413.3 [M+H]⁺.

### Example 142

### 2-(3-methyl-1H-pyrazol-4-yl)-4-(2,8-diazaspiro[4.5]decan-8-yl)pyrido[3,4-d]pyrimidine (Compound 142)

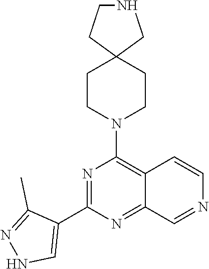 [SMILES: Cc1n[nH]cc1-c1nc(N2CCC3(CCNC3)CC2)c2ccncc2n1]

### Step 1: 3-methyl-1-((2-(trimethylsilyl)ethoxy)methyl)-1H-pyrazole-4-carbaldehyde

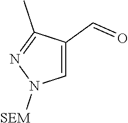 [SMILES: Cc1nn(COCC[Si](C)(C)C)cc1C=O]

To a stirred solution of 3-methyl-1H-pyrazole-4-carbaldehyde (5 g, 45.41 mmol) in THF (100 mL) was added NaH (2.0 g, 50 mmol, 60%) at 0° C. under nitrogen atmosphere. After 30 min, (2-(chloromethoxy)ethyl)trimethylsilane (10.26 g, 40.54 mmol) was added. The reaction was allowed to warm to room temperature and stirred for 16 h. The reaction was poured into sat. aq. NH₄Cl (50 mL), extracted with EtOAc (150 mL×2). The combined organic layers were washed with brine (100 mL), dried over anhydrous Na₂SO₄, filtered and concentrated in vacuo. The residue was purified by silica gel chromatography (solvent gradient: 0-20% EtOAc in petroleum ether) to give the title compound (10.0 g, 92%) as colorless oil. ¹H NMR (400 MHz, CDCl₃) δ 9.96-9.94 (m, 1H), 8.05-7.88 (m, 1H), 5.53-5.36 (m, 2H), 3.65-3.56 (m, 2H), 2.69-2.50 (m, 3H), 0.98-0.90 (m, 2H), 0.05-0.01 (m, 9H).

### Step 2: 2-(3-methyl-1-((2-(trimethylsilyl)ethoxy)methyl)-1H-pyrazol-4-yl)pyrido[3,4-d]pyrimidin-4-ol

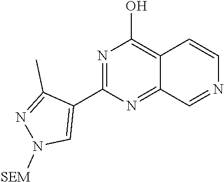 [SMILES: Cc1nn(COCC[Si](C)(C)C)cc1-c1nc(O)c2ccncc2n1]

To a solution of 3-aminopyridine-4-carboxamide (5 g, 36.46 mmol) in DMA (50 mL) was added 3-methyl-1-((2-(trimethylsilyl)ethoxy)methyl)-1H-pyrazole-4-carbaldehyde (10.5 g, 43.75 mmol) and CuO (5.8 g, 72.92 mmol). The mixture was heated to 135° C. for 40 hours under oxygen atmosphere. After cooling to room temperature, the reaction was poured into water (500 mL), the suspension was filtered and the filter cake was dried in vacuo to give the title compound (9.1 g, 70%) as a yellow solid. ¹H NMR (400 MHz, CDCl₃) δ 12.51 (s, 1H), 9.02 (s, 1H), 8.69 (s, 0.5H), 8.59 (s, 1H), 8.31 (s, 0.5H), 7.92 (s, 1H), 5.52-5.34 (m, 2H), 3.56-3.54 (m, 2H), 2.79-2.55 (m, 3H), 0.89-0.82 (m, 2H), −0.03-0.05 (m, 9H). LCMS (ESI) m/z: 358.3 [M+H]⁺.

### Step 3: 2-(3-methyl-1H-pyrazol-4-yl)-4-(2,8-diazaspiro[4.5]decan-8-yl)pyrido[3,4-d1pyrimidine

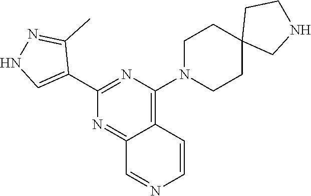 [SMILES: Cc1n[nH]cc1-c1nc(N2CCC3(CCNC3)CC2)c2ccncc2n1]

To a solution of 2-(3-methyl-1-((2-(trimethylsilyl)ethoxy)methyl)-1H-pyrazol-4-yl)pyrido[3,4-d]pyrimidin-4-ol (200 mg, 0.56 mmol) in DMF (5 mL) was added DIEA (0.22 mL, 1.12 mmol) and 2,4,6-triisopropylbenzenesulfonylchloride (200 mg, 0.67 mmol). The reaction mixture was stirred at room temperature for 1 hour. And then tert-butyl 2,8-diazaspiro[4.5]decane-2-carboxylate (135 mg, 0.56 mmol) was added to this reaction mixture. The reaction mixture was stirred at room temperature for 16 hours. The mixture was diluted with EtOAc (50 mL), washed with water (30 mL×3) and brine (30 mL). The organic layer was dried over anhydrous Na₂SO₄, filtered and concentrated in vacuo. The crude residue was purified by silica gel chromatography (solvent gradient: 0-5% MeOH in DCM) to give tert-butyl 8-(2-(3-methyl-1-((2-(trimethylsilyl)ethoxy)methyl)-1H-pyrazol-4-yl)pyrido[3,4-d]pyrimidin-4-yl)-2,8-diazaspiro[4.5]decane-2-carboxylate (200 mg, 62%) as a yellow solid. This residue was treated with 1 mL DCM and 1 mL TFA and allowed to stir at room temperature for 2 hours. The reaction mixture was then concentrated in vacuo, and then concentrated 2× further from DCM (5 mL) to remove residual TFA. The crude residue was then purified by HPLC to furnish the title compound. ¹H NMR (400 MHz, DMSO) δ 12.78 (br s, 1H), 9.08 (d, J=2.1 Hz, 1H), 8.47-8.41 (m, 1H), 8.14 (s, 1H), 7.80-7.73 (m, 1H), 3.97-3.74 (m, 4H), 3.42-3.35 (m, 4H), 2.85 (t, J=7.1 Hz, 1H), 2.69-2.60 (m, 5H), 1.87-1.79 (m, 1H), 1.76-1.65 (m, 2H), 1.60 (t, J=7.1 Hz, 1H). LCMS (ESI) m/z: 350.2 [M+H]⁺.

### Example 143

### 2-(3-methyl-1H-pyrazol-4-yl)-4-(2-methyl-2,8-diazaspiro[4.5]decan-8-yl)pyrido[3,4-d]pyrimidine (Compound 143)

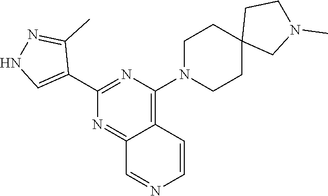 [SMILES: Cc1n[nH]cc1-c1nc(N2CCC3(CCN(C)C3)CC2)c2ccncc2n1]

Following the procedure described in Example 102 and making non-critical variations as required to replace 4-(2,8-diazaspiro[4.5]decan-8-yl)-2-(4-pyridyl)pyrido[3,4-d]pyrimidine hydrochloride with 2-(3-methyl-1H-pyrazol-4-yl)-4-(2,8-diazaspiro[4.5]decan-8-yl)pyrido[3,4-d]pyrimidine hydrochloride, the title compound was obtained as a yellow solid. ¹H NMR (400 MHz, DMSO-d₆) δ 9.06 (s, 1H), 8.42 (d, J=5.6 Hz, 2H), 8.13 (s, 1H), 7.72 (d, J=5.6 Hz, 1H), 3.86-3.66 (m, 4H), 2.64 (s, 3H), 2.51-2.46 (m, 2H), 2.36 (s, 2H), 2.22 (s, 3H), 1.74-1.62 (m, 6H). LCMS (ESI) m/z: 364.2 [M+H]⁺.

### Examples 144 and 145

### (S)-1-(8-(2-(3-methyl-1H-pyrazol-4-yl)pyrido[3,4-d]pyrimidin-4-yl)-2,8-diazaspiro[4.5]decan-2-yl)propan-2-ol and (R)-1-(8-(2-(3-methyl-1H-pyrazol-4-yl)pyrido[3,4-d]pyrimidin-4-yl)-2,8-diazaspiro[4.5]decan-2-yl)propan-2-ol (Compounds 144 and 145)

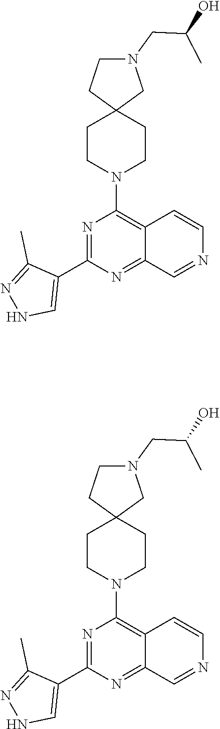 [SMILES: Cc1n[nH]cc1-c1nc(N2CCC3(CCN(C[C@H](C)O)C3)CC2)c2ccncc2n1 ; Cc1n[nH]cc1-c1nc(N2CCC3(CCN(C[C@@H](C)O)C3)CC2)c2ccncc2n1]

### Step 1: 1-(8-(2-(3-methyl-1H-pyrazol-4-yl)pyrido[3,4-d]pyrimidin-4-yl)-2,8-diazaspiro[4.5]decan-2-yl)propan-2-ol

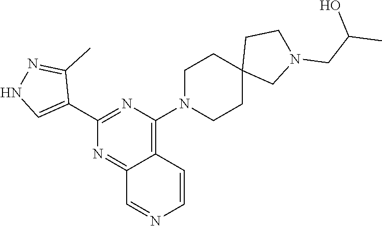 [SMILES: Cc1n[nH]cc1-c1nc(N2CCC3(CCN(CC(C)O)C3)CC2)c2ccncc2n1]

Following the procedure described in Example 113 and making non-critical variations as required to replace 4-(2,8-diazaspiro[4.5]decan-8-yl)-2-(4-pyridyl)pyrido[3,4-d]pyrimidine hydrochloride with 2-(3-methyl-1H-pyrazol-4-yl)-4-(2,8-diazaspiro[4.5]decan-8-yl)pyrido[3,4-d]pyrimidine hydrochloride, the title compound was obtained as a yellow solid. LCMS (ESI) m/z: 408.1 [M+H]⁺.

### Step 2: (S)-1-(8-(2-(3-methyl-1H-pyrazol-4-yl)pyrido[3,4-d]pyrimidin-4-yl)-2,8-diazaspiro[4.5]decan-2-yl)propan-2-ol and (R)-1-(8-(2-(3-methyl-1H-pyrazol-4-yl)pyrido[3,4-d]pyrimidin-4-yl)-2,8-diazaspiro[4.5]decan-2-yl)propan-2-ol

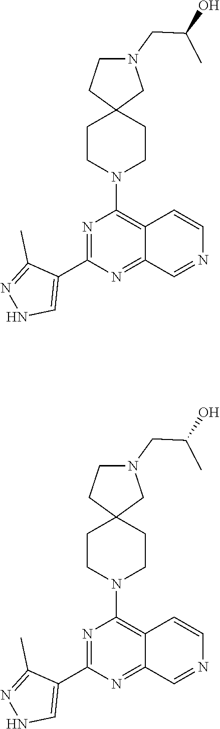 [SMILES: Cc1n[nH]cc1-c1nc(N2CCC3(CCN(C[C@H](C)O)C3)CC2)c2ccncc2n1 ; Cc1n[nH]cc1-c1nc(N2CCC3(CCN(C[C@@H](C)O)C3)CC2)c2ccncc2n1]

1-(8-(2-(3-methyl-1H-pyrazol-4-yl)pyrido[3,4-d]pyrimidin-4-yl)-2,8-diazaspiro[4.5]decan-2-yl)propan-2-ol (110 mg, 0.27 mmol) was separated by using chiral SFC (Phenomenex-Cellulose-2 (250 mm*30 mm, 10 um), Supercritical CO₂/EtOH+0.1% NH₄OH=50/50; 80 mL/min)) to give the title compounds, both as white solids. Absolute configuration was arbitrarily assigned to each enantiomer. Example 144 (18 mg, second peak): ¹H NMR (400 MHz, DMSO-d₆) δ 12.87 (s, 1H), 9.07 (s, 1H), 8.44 (d, J=6.0 Hz, 1H), 8.06 (s, 1H), 7.75 (d, J=5.6 Hz, 1H), 4.32 (s, 1H), 3.89-3.80 (m, 2H), 3.79-3.65 (m, 3H), 2.67 (s, 2H), 2.60-2.57 (m, 2H), 2.47 (s, 3H), 2.36-2.27 (m, 2H), 1.79-1.68 (m, 4H), 1.68-1.62 (m, 2H), 1.05 (d, J=6.4 Hz, 3H). LCMS (ESI) m/z: 408.2 [M+H]⁺. Example 145 (15 mg, first peak): ¹H NMR (400 MHz, DMSO-d₆) δ 12.86 (s, 1H), 9.07 (s, 1H), 8.44 (d, J=5.6 Hz, 1H), 8.07 (s, 1H), 7.76 (d, J=6.0 Hz, 1H), 4.30 (d, J=4.0 Hz, 1H), 3.89-3.81 (m, 2H), 3.80-3.68 (m, 3H), 2.67 (s, 2H), 2.61-2.57 (m, 2H), 2.45 (s, 3H), 2.34-2.30 (m, 2H), 1.76-1.68 (m, 4H), 1.67-1.62 (m, 2H), 1.05 (d, J=6.0 Hz, 3H). LCMS (ESI) m/z: 408.2 [M+H]⁺.

### Example 146

### 2-(8-(2-(3-methyl-1H-pyrazol-4-yl)pyrido[3,4-d]pyrimidin-4-yl)-2,8-diazaspiro[4.5]decan-2-yl)ethan-1-ol (Compound 146)

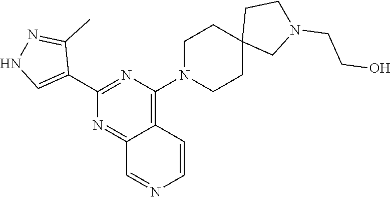 [SMILES: Cc1n[nH]cc1-c1nc(N2CCC3(CCN(CCO)C3)CC2)c2ccncc2n1]

Following the procedure described in Example 107 and making non-critical variations as required to replace 4-(2,8-diazaspiro[4.5]decan-8-yl)-2-(4-pyridyl)pyrido[3,4-d]pyrimidine hydrochloride and methyl 2-bromo-2-methylpropanoate with 2-(3-methyl-1H-pyrazol-4-yl)-4-(2,8-diazaspiro[4.5]decan-8-yl)pyrido[3,4-d]pyrimidine hydrochloride and 2-bromoethanol, the title compound was obtained as a yellow solid. ¹H NMR (400 MHz, DMSO-d₆) δ 12.86 (s, 1H), 9.07 (s, 1H), 8.44 (d, J=5.6 Hz, 1H), 8.13 (s, 1H), 7.75 (d, J=5.6 Hz, 1H), 4.41 (s, 1H), 3.87-3.73 (m, 4H), 3.47 (t, J=6.4 Hz, 2H), 2.65 (s, 3H), 2.57 (t, J=6.4 Hz, 2H), 2.48-2.43 (m, 4H), 1.76-1.63 (m, 6H). LCMS (ESI) m/z: 394.1 [M+H]⁺.

### Example 147

### 4-(2-cyclopentyl-2,8-diazaspiro[4.5]decan-8-yl)-2-(3-methyl-1H-pyrazol-4-yl)pyrido[3,4-d]pyrimidine (Compound 147)

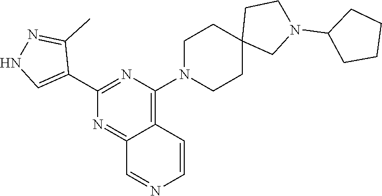 [SMILES: Cc1n[nH]cc1-c1nc(N2CCC3(CC2)CCN(C2CCCC2)C3)c2ccncc2n1]

Following the procedure described in Example 106 and making non-critical variations as required to replace 4-(2,8-diazaspiro[4.5]decan-8-yl)-2-(4-pyridyl)pyrido[3,4-d]pyrimidine hydrochloride with 2-(3-methyl-1H-pyrazol-4-yl)-4-(2,8-diazaspiro[4.5]decan-8-yl)pyrido[3,4-d]pyrimidine hydrochloride, the title compound was obtained as a yellow solid. ¹H NMR (400 MHz, DMSO-d₆) δ 12.87 (s, 1H), 9.07 (s, 1H), 8.43 (d, J=6.0 Hz, 1H), 8.09 (s, 1H), 7.75 (d, J=6.0 Hz, 1H), 4.07-4.05 (m, 1H), 3.88-3.82 (m, 2H), 3.78-3.71 (m, 2H), 2.67-2.62 (m, 4H), 1.83-1.55 (m, 10H), 1.54-1.29 (m, 4H). LCMS (ESI) m/z: 418.2 [M+H]⁺.

### Example 148

### 2-(3-methyl-1H-pyrazol-4-yl)-4-(2-(tetrahydrofuran-3-yl)-2,8-diazaspiro[4.5]decan-8-yl)pyrido[3,4-d]pyrimidine (Compound 148)

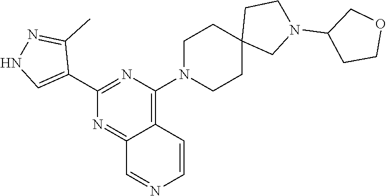 [SMILES: Cc1n[nH]cc1-c1nc(N2CCC3(CC2)CCN(C2CCOC2)C3)c2ccncc2n1]

Following the procedure described in Example 103 and making non-critical variations as required to replace 4-(2,8-diazaspiro[4.5]decan-8-yl)-2-(4-pyridyl)pyrido[3,4-d]pyrimidine hydrochloride and hydroxyacetone with 2-(3-methyl-1H-pyrazol-4-yl)-4-(2,8-diazaspiro[4.5]decan-8-yl)pyrido[3,4-d]pyrimidine hydrochloride and 3-oxotetrahydrofuran, the title compound was obtained as a yellow solid. ¹H NMR (400 MHz, DMSO-d₆) δ 12.86 (s, 1H), 9.07 (s, 1H), 8.44 (d, J=5.6 Hz, 1H), 8.09 (s, 1H), 7.75 (d, J=5.6 Hz, 1H), 3.89-3.80 (m, 2H), 3.79-3.74 (m, 2H), 3.74-3.68 (m, 2H), 3.68-3.62 (m, 1H), 3.49 (m, 1H), 2.85-2.75 (m, 1H), 2.69-2.62 (m, 1H), 2.65 (s, 2H), 2.60-2.54 (m, 2H), 2.47-2.40 (m, 2H), 1.97-1.89 (m, 1H), 1.82-1.75 (m, 1H), 1.74-1.64 (m, 6H). LCMS (ESI) m/z: 420.1 [M+H].

### Example 149

### 3-(8-(2-(3-methyl-1H-pyrazol-4-yl)pyrido[3,4-d]pyrimidin-4-yl)-2,8-diazaspiro[4.5]decan-2-yl)propan-1-ol (Compound 149)

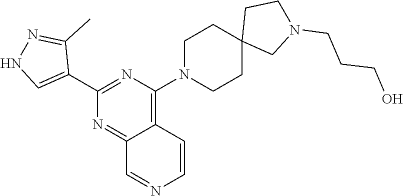 [SMILES: Cc1n[nH]cc1-c1nc(N2CCC3(CCN(CCCO)C3)CC2)c2ccncc2n1]

Following the procedure described in Example 107 and making non-critical variations as required to replace 4-(2,8-diazaspiro[4.5]decan-8-yl)-2-(4-pyridyl)pyrido[3,4-d]pyrimidine hydrochloride and methyl 2-bromo-2-methylpropanoate with 2-(3-methyl-1H-pyrazol-4-yl)-4-(2,8-diazaspiro[4.5]decan-8-yl)pyrido[3,4-d]pyrimidine hydrochloride and 3-bromo-1-propanol, the title compound was obtained as a white solid. ¹H NMR (400 MHz, DMSO-d₆) δ 9.06 (s, 1H), 8.43 (d, J=5.6 Hz, 1H), 8.14 (s, 1H), 7.73 (d, J=5.6 Hz, 1H), 3.86-3.80 (m, 2H), 3.77-3.71 (m, 2H), 3.45 (t, J=6.4 Hz, 2H), 2.65 (s, 3H), 2.55-2.51 (m, 2H), 2.44-2.37 (m, 4H), 1.76-1.63 (m, 6H), 1.61-1.54 (m, 2H). LCMS (ESI) m/z: 408.2 [M+H]⁺.

### Example 150

### 2-methyl-1-(8-(2-(3-methyl-1H-pyrazol-4-yl)pyrido[3,4-d]pyrimidin-4-yl)-2,8-diazaspiro[4.5]decan-2-yl)propan-2-o0 (Compound 150)

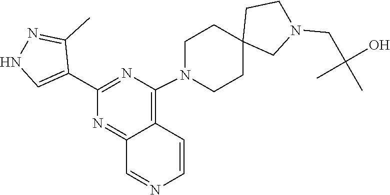 [SMILES: Cc1n[nH]cc1-c1nc(N2CCC3(CCN(CC(C)(C)O)C3)CC2)c2ccncc2n1]

Following the procedure described in Example 110 and making non-critical variations as required to replace 4-(2,8-diazaspiro[4.5]decan-8-yl)-2-(4-pyridyl)pyrido[3,4-d]pyrimidine hydrochloride with 2-(3-methyl-1H-pyrazol-4-yl)-4-(2,8-diazaspiro[4.5]decan-8-yl)pyrido[3,4-d]pyrimidine hydrochloride, the title compound was obtained as a yellow solid. ¹H NMR (400 MHz, DMSO-d₆) δ 12.86 (s, 1H), 9.06 (s, 1H), 8.42 (d, J=5.6 Hz, 1H), 8.07 (s, 1H), 7.74 (d, J=5.6 Hz, 1H), 4.05 (s, 1H), 3.91-3.79 (m, 2H), 3.79-3.69 (m, 2H), 2.71-2.61 (m, 4H), 2.55 (s, 2H), 2.32 (s, 3H), 1.82-1.66 (m, 4H), 1.66-1.59 (m, 2H), 1.08 (s, 6H). LCMS (ESI) m/z: 422.1 [M+H]⁺.

### Example 151

### 4-(2-ethyl-2,8-diazaspiro[4.5]decan-8-yl)-2-(5-methyl-1H-pyrazol-4-yl)pyrido[3,4-d]pyrimidine (Compound 151)

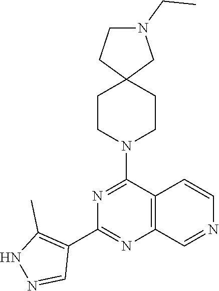 [SMILES: CCN1CCC2(CCN(c3nc(-c4cn[nH]c4C)nc4cnccc34)CC2)C1]

Following the procedure described in Example 138 and making non-critical variations as required to replace 4-(2,8-diazaspiro[4.5]decan-8-yl)-2-(4-pyridyl)pyrido[3,4-d]pyrimidine hydrochloride with 2-(3-methyl-1H-pyrazol-4-yl)-4-(2,8-diazaspiro[4.5]decan-8-yl)pyrido[3,4-d]pyrimidine hydrochloride, the title compound was obtained as a yellow solid. ¹H NMR (400 MHz, DMSO-d₆) δ 12.84 (s, 1H), 9.07 (s, 1H), 8.43 (d, J=5.6 Hz, 1H), 8.11 (s, 1H), 7.75 (d, J=5.6 Hz, 1H), 3.88-3.72 (m, 4H), 2.65 (s, 3H), 2.55-2.51 (m, 2H), 2.41 (s, 2H), 2.39-2.36 (m, 2H), 1.74-1.64 (m, 6H), 1.02 (t, J=7.2 Hz, 3H). LCMS (ESI) m/z: 378.1 [M+H]⁺.

### Example 152

### 2-(3-methyl-1H-pyrazol-4-yl)-4-(2-(oxetan-3-ylmethyl)-2,8-diazaspiro[4.5]decan-8-yl)pyrido[3,4-d]pyrimidine (Compound 152)

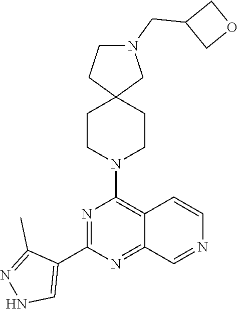 [SMILES: Cc1n[nH]cc1-c1nc(N2CCC3(CCN(CC4COC4)C3)CC2)c2ccncc2n1]

Following the procedure described in Example 103 and making non-critical variations as required to replace 4-(2,8-diazaspiro[4.5]decan-8-yl)-2-(4-pyridyl)pyrido[3,4-d]pyrimidine hydrochloride and hydroxyacetone with 2-(3-methyl-1H-pyrazol-4-yl)-4-(2,8-diazaspiro[4.5]decan-8-yl)pyrido[3,4-d]pyrimidine hydrochloride and oxetane-3-carbaldehyde, the title compound was obtained as a yellow solid. ¹H NMR (400 MHz, DMSO-d₆) δ 12.86 (s, 1H), 9.07 (s, 1H), 8.44 (d, J=6.0 Hz, 1H), 8.06 (s, 1H), 7.75 (d, J=5.6 Hz, 1H), 4.66-4.62 (m, 2H), 4.28-4.25 (m, 2H), 3.89-3.81 (m, 2H), 3.78-3.72 (m, 2H), 3.20-3.02 (m, 1H), 2.74-2.65 (m, 4H), 2.43-2.27 (m, 2H), 1.73-1.62 (m, 6H). LCMS (ESI) m/z: 420.2 [M+H]⁺.

### Example 153

### 2-(5-chloro-1H-pyrazol-4-yl)-4-(2,8-diazaspiro[4.5]decan-8-yl)pyrido[3,4-d]pyrimidine (Compound 153)

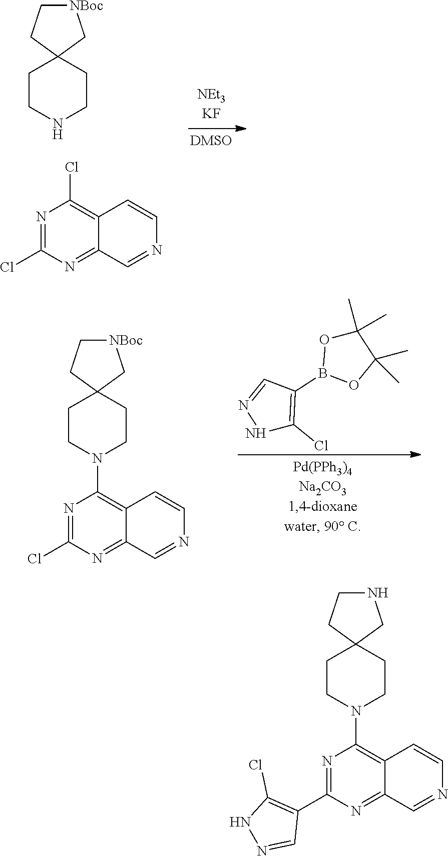

### Step 1: tert-butyl 8-(2-chloropyrido[3,4-d]pyrimidin-4-yl)-2,8-diazaspiro[4.5]decane-2-carboxylate

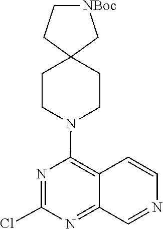 [SMILES: CC(C)(C)OC(=O)N1CCC2(CCN(c3nc(Cl)nc4cnccc34)CC2)C1]

2,4-dichloropyrido[3,4-d]pyrimidine (800 mg, 4 mmol, 1 equiv.), potassium fluoride (700 mg, 10 mmol, 3 equiv.), and tert-butyl 2,8-diazaspiro[4.5]decane-2-carboxylate (1000 mg, 4 mmol, 1 equiv.) were added to a 40 mL vial. dimethyl sulfoxide (10 mL, 0.3 M) was added followed by triethylamine (3 mL, 20 mmol, 5 equiv.), and the reaction was allowed to stir at room temperature for 1 hour. After monitoring the reaction via LCMS, the reaction had gone to completion at this time. The mixture was transferred to a separatory funnel, and diluted with EtOAc (15 mL), aqueous saturated NH₄Cl (10 mL) and water (10 mL). The layers were separated, and the aqueous layer was extracted with further EtOAc (3×20 mL). The combined organic extracts dried over Na₂SO₄, filtered, and concentrated in vacuo. The crude organic residue was flashed via 24 g Isco cartridge eluting 0 to 15% MeOH in DCM to furnish tert-butyl 8-(2-chloropyrido[3,4-d]pyrimidin-4-yl)-2,8-diazaspiro[4.5]decane-2-carboxylate (1200 mg, 74% Yield). LCMS (ESI) m/z: 426.05 [M+Na]⁺.

### Step 2: 2-(5-chloro-1H-pyrazol-4-yl)-4-(2,8-diazaspiro[4.5]decan-8-yl)pyrido[3,4-d]pyrimidine

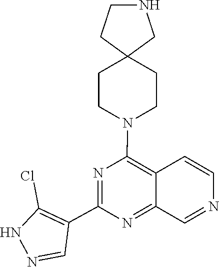 [SMILES: Clc1[nH]ncc1-c1nc(N2CCC3(CCNC3)CC2)c2ccncc2n1]

tert-butyl 8-(2-chloropyrido[3,4-d]pyrimidin-4-yl)-2,8-diazaspiro[4.5]decane-2-carboxylate (190 mg, 0.470 mmol, 1 equiv.), tetrakis(triphenylphosphine)palladium(0) (54 mg, 0.047 mmol, 0.1 equiv.), sodium carbonate (150 mg, 1.40 mmol, 3 equiv), 3-chloro-4-(4,4,5,5-tetramethyl-1,3,2-dioxaborolan-2-yl)-1H-pyrazole (166 mg, 0.706 mmol, 1.5 equiv) were added to a 2-dram vial. The vial was purged with N₂, and then 1,4-dioxane (2.35 mL) was added followed by water (0.24 mL), and the reaction mixture was sparged with N₂ for 5 minutes. The vial was then sealed and heated to 90° C. for 16 hours. The reaction was then cooled to room temperature, transferred to a 20 mL vial, and diluted with water (5 mL) and EtOAc (5 mL). The layers were separated, and the aqueous was extracted with further EtOAc (4×5 mL). The combined organic extracts were dried over Na₂SO₄, filtered, and concentrated in vacuo. The crude residue was dissolved in DCM (1 mL) and TFA (1 mL), and let stir for 1 hour at room temperature. The reaction mixture was then concentrated in vacuo, and then concentrated 2× further from DCM (5 mL) to remove residual TFA. The crude residue was then purified by HPLC to fumish 2-(5-chloro-1H-pyrazol-4-yl)-4-(2,8-diazaspiro[4.5]decan-8-yl)pyrido[3,4-d]pyrimidine (47 mg, 0.127 mmol, 27% yield). LCMS (ESI) m/z: 370.1 [M+H]⁺. ¹H NMR (400 MHz, DMSO-d₆) δ 9.09 (s, 1H), 8.54-8.44 (m, 3H), 7.81 (d, J=5.7 Hz, 1H), 4.02-3.82 (m, 4H), 2.89 (t, J=7.1 Hz, 2H), 2.71 (s, 2H), 1.82 (t, J=7.1 Hz, 1H), 1.74-1.67 (m, 4H), 1.63 (t, J=7.1 Hz, 2H).

### Example 154

### 4-(2,8-diazaspiro[4.5]decan-8-yl)-2-(5-(trifluoromethyl)-1H-pyrazol-4-yl)pyrido[3,4-d]pyrimidine (Compound 154)

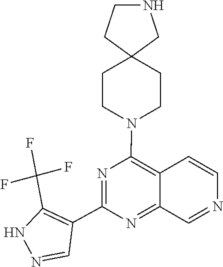 [SMILES: FC(F)(F)c1[nH]ncc1-c1nc(N2CCC3(CCNC3)CC2)c2ccncc2n1]

Following the procedure described in Example 153, Step 2 and making non-critical variations as required to replace 3-chloro-4-(4,4,5,5-tetramethyl-1,3,2-dioxaborolan-2-yl)-1H-pyrazole with 4-(4,4,5,5-tetramethyl-1,3,2-dioxaborolan-2-yl)-5-(trifluoromethyl)-1H-pyrazole the title compound was obtained (44 mg, 24% yield). LCMS (ESI) m/z: 404.1 [M+H]⁺. ¹H NMR (400 MHz, DMSO-d6) δ 10.1 (br s, 1H) 9.09 (s, 1H), 8.63 (s, 1H), 8.50 (d, J=5.6 Hz, 1H), 7.82 (d, J=6.0 Hz, 1H), 4.01-3.81 (m, 4H), 2.91 (t, J=7.1 Hz, 2H), 2.71 (s, 2H), 1.66 (dt, J=18.4, 6.5 Hz, 6H). Exchangeable amine NH proton not observed.

### Example 155

### 4-(4-(2,8-diazaspiro[4.5]decan-8-yl)pyrido[3,4-d]pyrimidin-2-yl)-1H-pyrazole-5-carbonitrile (Compound 155)

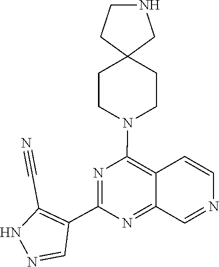 [SMILES: N#Cc1[nH]ncc1-c1nc(N2CCC3(CCNC3)CC2)c2ccncc2n1]

Following the procedure described in Example 153, Step 2 and making non-critical variations as required to replace 3-chloro-4-(4,4,5,5-tetramethyl-1,3,2-dioxaborolan-2-yl)-1H-pyrazole with 4-(4,4,5,5-tetramethyl-1,3,2-dioxaborolan-2-yl)-1-((2-(trimethylsilyl)ethoxy)methyl)-1H-pyrazole-5-carbonitrile the title compound was obtained (5 mg, 15%) as a yellow solid. ¹H NMR (400 MHz, DMSO-d₆) δ 9.08 (s, 1H), 8.56 (s, 1H), 8.48 (d, J=5.6 Hz, 1H), 7.82 (d, J=5.6 Hz, 1H), 4.00-3.91 (m, 4H), 2.99-2.94 (m, 2H), 2.77 (s, 2H), 1.72-1.61 (m, 6H). LCMS (ESI) m/z: 361.1 [M+H]⁺.

### Example 156

### 2-(1H-pyrrolo[2,3-b]pyridin-4-yl)-4-(2,8-diazaspiro[4.5]decan-8-yl)pyrido[3,4-d]pyrimidine (Compound 156)

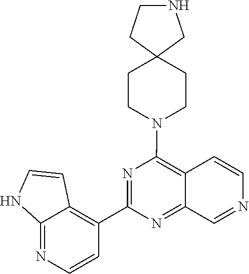 [SMILES: c1cc2c(N3CCC4(CCNC4)CC3)nc(-c3ccnc4[nH]ccc34)nc2cn1]

Following the procedure described in Example 153, Step 2 and making non-critical variations as required to replace 3-chloro-4-(4,4,5,5-tetramethyl-1,3,2-dioxaborolan-2-yl)-1H-pyrazole with 4-(4,4,5,5-tetramethyl-1,3,2-dioxaborolan-2-yl)-1H-pyrrolo[2,3-b]pyridine the title compound was obtained. ¹H NMR (400 MHz, DMSO-d₆) 11.83 (s, 1H), 9.33 (d, J=0.7 Hz, 1H), 8.59 (d, J=5.6 Hz, 1H), 8.38 (d, J=5.0 Hz, 1H), 8.13 (d, J=5.0 Hz, 1H), 7.94-7.84 (m, 1H), 7.68-7.61 (m, 1H), 7.48 (d, J=3.4 Hz, 1H), 4.11-3.84 (m, 4H), 3.44-3.29 (m, 3H), 2.98 (t, J=7.1 Hz, 1H), 1.85 (t, J=7.1 Hz, 1H), 1.81-1.75 (m, 4H), 1.71 (t, J=7.2 Hz, 1H). Exchangeable amine NH proton not observed. LCMS (ESI) m/z: 386.1 [M+H]⁺.

### Example 157

### 6-benzyl-2-(pyridin-4-yl)-4-(2,8-diazaspiro[4.5]decan-8-yl)pyrido[3,4-d]pyrimidine (Compound 157)

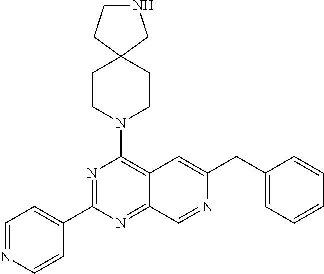 [SMILES: c1ccc(Cc2cc3c(N4CCC5(CCNC5)CC4)nc(-c4ccncc4)nc3cn2)cc1]

### Step 1: 6-chloro-2-(pyridin-4-yl)pyrido[3,4-d]pyrimidin-4-ol

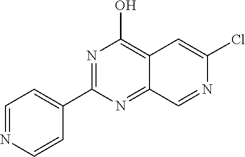 [SMILES: Oc1nc(-c2ccncc2)nc2cnc(Cl)cc12]

To a solution of potassium 2-methyl-2-butoxide (22.02 g, 174.46 mmol) in THF (100 mL) was added a solution of ethyl 5-amino-2-chloro-pyridine-4-carboxylate (14 g, 69.78 mmol) (prepared according to the procedure in US2016176871) and 4-cyanopyridine (8.72 g, 83.74 mmol) in THF (300 mL) dropwise (˜4 mL/min) at 0° C. The reaction was allowed to warm to room temperature and stirred for 16 h. Water (40 mL) and acetic acid (10 mL) were added. The mixture was stirred at room temperature for 20 minutes, the resulting yellow precipitate was filtered and the solid was washed with water (30 mL×2) to give the title compound (11 g, 55%) as a yellow solid. ¹H NMR (400 MHz, DMSO-d₆) δ 8.96 (s, 1H), 8.81-8.77 (m, 2H), 8.11-8.07 (m, 2H), 7.99 (s, 1H). LCMS (ESI) m/z: 259.1 [M+H]⁺.

### Step 2: 6-chloro-2-(pyridin-4-yl)-4-((2-(trimethylsilyl)ethoxy)methoxy)pyrido[3,4-d]pyrimidine

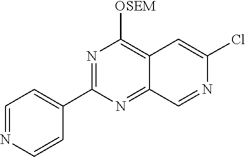

To a solution of 6-chloro-2-(4-pyridyl)pyrido[3,4-d]pyrimidin-4-ol (10 g, 38.66 mmol) and triethylamine (27 mL, 193 mmol) in DCM (100 mL) was added 2-(trimethylsilyl)ethoxymethyl chloride (27 mL, 155 mmol) in DCM (100 mL) dropwise at 0° C. The mixture was heated to 45° C. for 16 hours under nitrogen atmosphere. After cooling to room temperature, the mixture was diluted with DCM (200 mL), washed with sat. aq. NaHCO₃ (150 mL) and brine (150 mL). The organic layer was dried over anhydrous Na₂SO₄, filtered and concentrated in vacuo. The crude residue was purified by silica gel chromatography (solvent gradient: 0-5% EtOAc in petroleum ether) to give the title compound (15 g, 99%) as yellow oil. ¹H NMR (400 MHz, CDCl₃) δ 8.96 (s, 1H), 8.85-8.80 (m, 2H), 8.14 (s, 1H), 7.72-7.68 (m, 2H), 5.27 (s, 2H), 3.88-3.65 (m, 2H), 1.05-0.88 (m, 2H), 0.02 (s, 9H). LCMS (ESI) m/z: 389.2 [M+H]⁺.

### Step 3: 6-benzyl-2-(pyridin-4-yl)-4-((2-(trimethylsilyl)ethoxy)methoxy)pyrido[3,4-d]pyrimidine

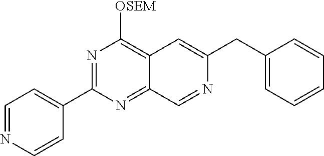

To a solution of 6-chloro-2-(pyridin-4-yl)-4-((2-(trimethylsilyl)ethoxy)methoxy)pyrido [3,4-d]pyrimidine (3 g, 7.71 mmol) and (2-dicyclohexylphosphino-2′,4′,6′-triisopropyl-1,1′-biphenyl)[2-(2′-amino-1,1′-biphenyl)]palladium(II)methanesulfonate (330 mg, 0.39 mmol) in THF (15 mL) was added benzylzinc(II) chloride (19.5 mL, 9.75 mmol) (0.5M in THF) (prepared according to the procedure in WO2019123011) dropwise under nitrogen atmosphere. The mixture was stirred at room temperature for 5 hours. The reaction was quenched with water (50 mL) and extracted with EtOAc (100 mL×2). The combined organic layers were dried over anhydrous Na₂SO₄, filtered and concentrated in vacuo. The crude residue was purified by silica gel chromatography (solvent gradient: 0-70% EtOAc in petroleum ether) to give the title compound (1.85 g, 54%) as yellow oil. ¹H NMR (400 MHz, CDCl₃) δ 9.14 (s, 1H), 8.96 (d, J=5.6 Hz, 2H), 8.03-7.94 (m, 3H), 7.38-7.29 (m, 4H), 7.27-7.22 (m, 1H), 5.27 (s, 2H), 4.35 (s, 2H), 3.84-3.74 (m, 2H), 1.02-0.94 (m, 2H), 0.03 (s, 9H). LCMS (ESI) m/z: 445.1 [M+H]⁺.

### Step 4: 6-benzyl-2-(pyridin-4-yl)pyrido[3,4-d]pyrimidin-4-ol

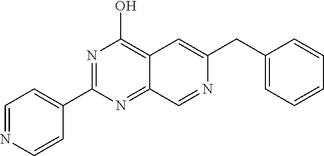 [SMILES: Oc1nc(-c2ccncc2)nc2cnc(Cc3ccccc3)cc12]

To a solution of 6-benzyl-2-(pyridin-4-yl)-4-((2-(trimethylsilyl)ethoxy)methoxy)pyrido [3,4-d]pyrimidine (1 g, 1.8 mmol) in DCM (20 mL) was added trifluoroacetic acid (5 mL, 6.73 mmol). The mixture was stirred at room temperature for 16 hours. The mixture was concentrated in vacuo to give the title compound (700 mg, crude) as a brown solid that required no further purification. LCMS (ESI) m/z: 314.9 [M+H]⁺.

### Step 5: 6-benzyl-4-chloro-2-(pyridin-4-yl)pyrido[3,4-d]pyrimidine

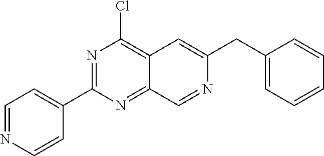 [SMILES: Clc1nc(-c2ccncc2)nc2cnc(Cc3ccccc3)cc12]

A solution of 6-benzyl-2-(pyridin-4-yl)pyrido[3,4-d]pyrimidin-4-ol (700 mg, 2.23 mmol) in phosphorus oxychloride (5 mL) was heated to 110° C. for 16 hours. After cooling to room temperature, the mixture was concentrated in vacuo and the crude residue was dissolved in DCM (100 mL) and basified with sat. aq. NaHCO₃ (50 mL) to pH 8 at 0° C. The organic layer was dried over anhydrous Na₂SO₄, filtered and concentrated in vacuo to give the title compound (6.7 g, crude) as a black solid. LCMS (ESI) m/z: 333.1 [M+H]⁺.

### Step 6: tert-butyl 8-(6-benzyl-2-(pyridin-4-yl)pyrido[3,4-d]pyrimidin-4-yl)-2,8-diazaspiro[4.5]decane-2-carboxylate

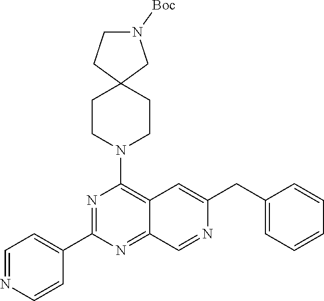 [SMILES: CC(C)(C)OC(=O)N1CCC2(CCN(c3nc(-c4ccncc4)nc4cnc(Cc5ccccc5)cc34)CC2)C1]

To a solution of 6-benzyl-4-chloro-2-(pyridin-4-yl)pyrido[3,4-d]pyrimidine (50 mg, 0.15 mmol) in 1-methyl-2-pyrrolidinone (2 mL) was added tert-butyl 2,8-diazaspiro[4.5]decane-2-carboxylate (40 mg, 0.17 mmol), triethylamine (8.6 mL, 61.81 mmol) and potassium fluoride (46 mg, 0.45 mmol). The mixture was heated to 80° C. for 16 hours under nitrogen atmosphere. After cooling to room temperature, the reaction was diluted with water (20 mL) and extracted with EtOAc (50 mL). The organic layer was washed with brine (20 mL×3), dried over anhydrous Na₂SO₄, filtered and concentrated in vacuo to give the title compound (74 mg, crude) as a yellow solid. LCMS (ESI) m/z: 537.4 [M+H].

### Step 7: 6-benzyl-2-(pyridin-4-yl)-4-(2,8-diazaspiro[4.5]decan-8-yl)pyrido[3,4-d]pyrimidine

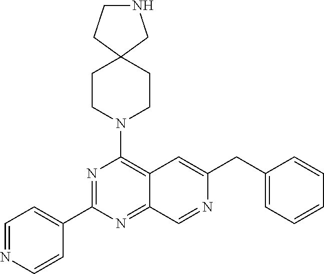 [SMILES: c1ccc(Cc2cc3c(N4CCC5(CCNC5)CC4)nc(-c4ccncc4)nc3cn2)cc1]

To a solution of tert-butyl 8-(6-benzyl-2-(pyridin-4-yl)pyrido[3,4-d]pyrimidin-4-yl)-2,8-diazaspiro[4.5]decane-2-carboxylate (74 mg, 0.14 mmol) in DCM (1.5 mL) was added trifluoroacetic acid (0.5 mL, 6.54 mmol). The mixture was stirred at room temperature for 2 hours. The mixture was concentrated in vacuo, the residue was purified by reverse phase chromatography (acetonitrile 10-40%/0.225% formic acid in water) to give the title compound (17 mg, 27%) as a yellow solid. ¹H NMR (400 MHz, DMSO-d₆) δ 9.21 (s, 1H), 8.75 (d, J=6.0 Hz, 2H), 8.39 (s, 1H), 8.33-8.26 (m, 2H), 7.67 (s, 1H), 7.35-7.29 (m, 4H), 7.25-7.19 (m, 1H), 4.29 (s, 2H), 3.95-3.81 (m, 4H), 3.23-3.14 (m, 2H), 2.99 (s, 2H), 1.86-1.80 (m, 2H), 1.77-1.68 (m, 4H). LCMS (ESI) m/z: 437.3 [M+H]⁺.

### Example 158

### 6-methyl-2-(pyridin-4-yl)-4-(2,8-diazaspiro[4.5]decan-8-yl)pyrido[3,4-d]pyrimidine (Compound 158) trifluoroacetate

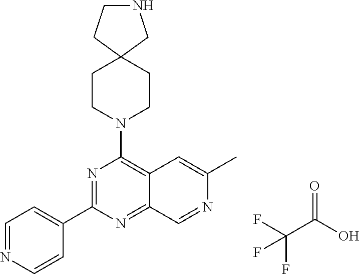 [SMILES: Cc1cc2c(N3CCC4(CCNC4)CC3)nc(-c3ccncc3)nc2cn1 ; O=C(O)C(F)(F)F]

### Step 1: 6-methyl-2-(pyridin-4-yl)-4-((2-(trimethylsilyl)ethoxy)methoxy)pyrido[3,4-d]pyrimidine

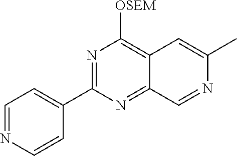

To a stirred solution of 6-chloro-2-(pyridin-4-yl)-4-((2-(trimethylsilyl)ethoxy)methoxy) pyrido[3,4-d]pyrimidine (600 mg, 1.54 mmol) in 1,4-dioxane (10 mL) and water (1 mL) was added methylboronic acid (462 mg, 7.71 mmol), Cs₂CO₃ (1.5 g, 4.63 mmol) and 1,1′-bis(diphenylphosphino)ferrocene palladium dichloride (115 mg, 0.15 mmol). The mixture was heated to 110° C. for 16 hours under nitrogen atmosphere. After cooling to room temperature, the reaction mixture was filtered and the filtrate was concentrated in vacuo. The crude residue was purified by silica gel chromatography (solvent gradient: 0-5% MeOH in DCM) to give the title compound (320 mg, 56%) as a yellow solid. LCMS (ESI) m/z: 369.2 [M+H]⁺.

### Step 2: 6-methyl-2-(pyridin-4-yl)-4-(2,8-diazaspiro[4.5]decan-8-yl)pyrido[3,4-d]pyrimidine trifluoroacetate

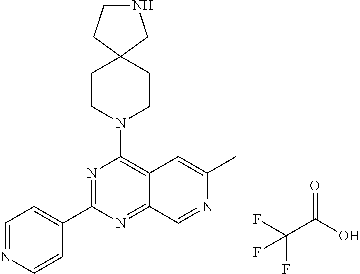 [SMILES: Cc1cc2c(N3CCC4(CCNC4)CC3)nc(-c3ccncc3)nc2cn1 ; O=C(O)C(F)(F)F]

Following the procedure described in Example 157 and making non-critical variations as required to replace 6-benzyl-2-(pyridin-4-yl)-4-((2-(trimethylsilyl)ethoxy)methoxy)pyrido [3,4-d]pyrimidine with 6-methyl-2-(pyridin-4-yl)-4-((2-(trimethylsilyl)ethoxy)methoxy) pyrido[3,4-d]pyrimidine, the title compound was obtained as a yellow solid. ¹H NMR (400 MHz, DMSO-d₆) δ 9.20 (s, 1H), 8.79-8.74 (m, 2H), 8.34-8.30 (m, 2H), 8.21 (s, 1H), 7.71 (s, 1H), 4.00-3.86 (m, 4H), 3.36-3.27 (m, 2H), 3.11 (s, 2H), 2.67 (s, 3H), 1.93-1.88 (m, 2H), 1.85-1.72 (m, 4H). LCMS (ESI) m/z: 361.4 [M+H].

### Example 159

### N-((2-(pyridin-4-yl)-4-(2,8-diazaspiro[4.5]decan-8-yl)pyrido[3,4-d]pyrimidin-6-yl)methyl)propionamide (Compound 159)

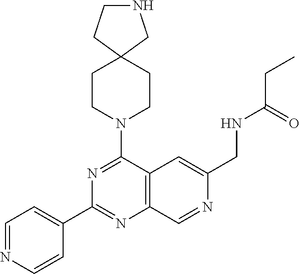 [SMILES: CCC(=O)NCc1cc2c(N3CCC4(CCNC4)CC3)nc(-c3ccncc3)nc2cn1]

### Step 1: Ethyl 5-amino-2-bromoisonicotinate

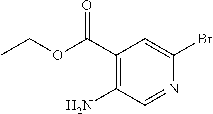

To a solution of ethyl 3-aminoisonicotinate (250.0 g, 1.50 mol) in DMF (4000 mL) was added NBS (281 g, 1.58 mol). The mixture was heated to 50° C. and stirred for 5 hours. After completion of reaction, it was cooled to room temperature, water (12.5 L) was added and the reaction mixture was extracted with EtOAc (5000 mL×3). The combined organics were washed with brine (10 L×3). Dried over anhydrous Na₂SO₄ and then concentrated under reduced pressure to give a crude which was purified by silica gel chromatography (Petroleum ether/EtOAc=10:1) to give ethyl 5-amino-2-bromoisonicotinate (254.4 g, 69.2%) as a yellow solid. LCMS (ESI) m/z: 245.0, 247.0 (Br pattern) [M+H]⁺. ¹H NMR (400 MHz, DMSO-d₆): δ 8.06 (d, J=0.4 Hz, 1H), 7.61 (s, 1H), 6.81 (brs, 2H), 4.33 (q, J=7.2 Hz, 2H), 1.32 (t, J=7.2 Hz, 3H).

### Step 2: 6-bromo-2-(4-pyridyl)pyrido[3,4-d]pyrimidin-4-ol

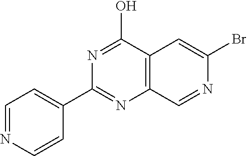 [SMILES: Oc1nc(-c2ccncc2)nc2cnc(Br)cc12]

Potassium 2-methyl-2-butoxide (158 g, 1.25 mol) was added in THF (930 mL). Then the solution of ethyl 5-amino-2-bromoisonicotinate (122.5 g, 499.8 mmol, 1.0 eq) and isonicotinonitrile (62.5 g, 599.8 mmol, 1.2 eq) in THF (2450 mL) were added dropwise at 0° C. under N₂. The mixture was stirred at room temperature for 2 hours. After completion of the reaction, water (6.2 L) and AcOH (93 mL) was added. The mixture was stirred at room temperature for 20 minutes. Then the solid was collected by filtration and washed with water (500 mL×3). The solid was dried to 6-bromo-2-(4-pyridyl)pyrido[3,4-d]pyrimidin-4-ol (two batches to give 145.0 g, 47.9%) as a yellow solid. LCMS (ESI) m/z: 300.9, 302.9 (Br pattern) [M−H]⁺. ¹H NMR (400 MHz, DMSO-d₆): δ 13.21 (s, 1H), 8.99 (s, 1H), 8.82 (q, J=3.2 Hz, 2H), 8.16-8.09 (m, 3H).

### Step 3: tert-butyl 8-(6-bromo-2-(pyridin-4-yl)pyrido[3,4-d]pyrimidin-4-yl)-2,8-diazaspiro[4.5]decane-2-carboxylate

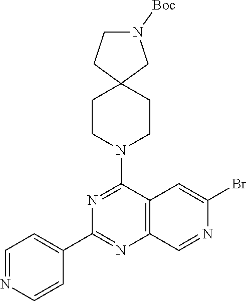 [SMILES: CC(C)(C)OC(=O)N1CCC2(CCN(c3nc(-c4ccncc4)nc4cnc(Br)cc34)CC2)C1]

To a solution of 6-bromo-2-(4-pyridyl)pyrido[3,4-d]pyrimidin-4-ol (200 mg, 0.66 mmol) in DMA (5 mL) was added 4-dimethylaminopyridine (8 mg, 0.07 mmol) and N,N-diisopropylethylamine (0.34 mL, 2.0 mmol). 2,4,6-triisopropylbenzenesulfonyl chloride (240 mg, 0.79 mmol) was finally added and the reaction mixture was stirred at room temperature for 30 minutes. tert-Butyl 2,8-diazaspiro[4.5]decane-2-carboxylate (190 mg, 0.79 mmol) was added and the reaction mixture was stirred at room temperature for 3 hours. A saturated solution of NaHCO₃ (25 mL) and ethyl acetate (40 mL) were added. The phases were separated and the organic layer was washed with water (30 mL), brine (30 mL), dried over Na₂SO₄, filtered and concentrated to a brown oil. The crude oil was purified by column chromatography on silica gel (MeOH/DCM) to provide the title compound tert-butyl 8-[6-bromo-2-(4-pyridyl)pyrido[3,4-d]pyrimidin-4-yl]-2,8-diazaspiro[4.5]decane-2-carboxylate (197 mg, 57% yield) as a brown gum. LCMS (ESI) m/z: 525-527 (Br pattern) [M+H]⁺.

### Step 4: tert-butyl 8-(2-(pyridin-4-yl)-6-vinylpyrido[3,4-d]pyrimidin-4-yl)-2,8-diazaspiro[4.5]decane-2-carboxylate

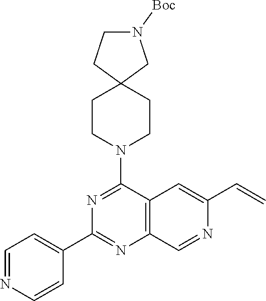 [SMILES: C=Cc1cc2c(N3CCC4(CCN(C(=O)OC(C)(C)C)C4)CC3)nc(-c3ccncc3)nc2cn1]

tert-butyl 8-[6-bromo-2-(4-pyridyl)pyrido[3,4-d]pyrimidin-4-yl]-2,8-diazaspiro[4.5]decane-2-carboxylate (197 mg, 0.38 mmol), and potassium vinyltrifluoroborate (56 mg, 0.42 mmol) were dissolved in 1,4-dioxane (3 mL) and the solution was degassed with a nitrogen flow for 10 minutes. Triethylamine (0.11 mL, 0.76 mmol) was added while the solution was being degassed for another 5 minutes. Then, [1,1′-bis(diphenylphosphino)ferrocene]dichloropalladium(II) (28 mg, 0.04 mmol) was added to the reaction mixture and it was capped under nitrogen and heated to 85° C. for 4 hours. The reaction mixture was cooled to room temperature and filtered through Celite. The filtrate was diluted with a saturated solution of sodium bicarbonate (15 mL) and it was extracted three times with ethyl acetate (20 mL). The organics were combined, dried with anhydrous sodium sulfate, filtered and concentrated to dryness. The crude residue was purified by column chromatography on silica gel (MeOH/DCM) to provide the title compound tert-butyl 8-[2-(4-pyridyl)-6-vinyl-pyrido[3,4-d]pyrimidin-4-yl]-2,8-diazaspiro[4.5]decane-2-carboxylate (106 mg, 59% yield) as a brown solid. LCMS (ESI) m/z: 473.1 [M+H]⁺.

### Step 5: tert-butyl 8-(6-formyl-2-(pyridin-4-yl)pyrido[3,4-d]pyrimidin-4-yl)-2,8-diazaspiro[4.5]decane-2-carboxylate

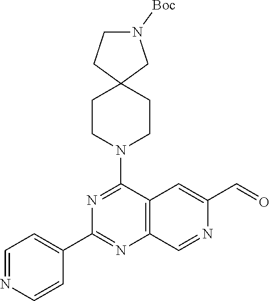 [SMILES: CC(C)(C)OC(=O)N1CCC2(CCN(c3nc(-c4ccncc4)nc4cnc(C=O)cc34)CC2)C1]

To a solution of tert-butyl 8-[2-(4-pyridyl)-6-vinyl-pyrido[3,4-d]pyrimidin-4-yl]-2,8-diazaspiro[4.5]decane-2-carboxylate (106 mg, 0.22 mmol) and NMO (53 mg, 0.45 mmol) in DCM (4 mL) under an atmosphere of nitrogen was added a solution of 4% wt osmium tetroxide in water (71 uL, 0.01 mmol). The reaction mixture was stirred for 16 hours at room temperature. After complete conversion of olefin to diol, sodium periodate (72 mg, 0.34 mmol) in water (2 mL) was added and the mixture was stirred for another 16 hours at room temperature. Reaction mixture was diluted with dichloromethane, washed water (100 mL), brine (100 mL), dried over anhydrous sodium sulfate and evaporated. The crude residue was purified by column chromatography on silica gel (MeOH/DCM) to provide the title compound tert-butyl 8-[6-formyl-2-(4-pyridyl)pyrido[3,4-d]pyrimidin-4-yl]-2,8-diazaspiro[4.5]decane-2-carboxylate (106 mg, >99% yield) as a yellow solid. LCMS (ESI) m/z: 475.1 [M+H]⁺.

### Step 6: tert-butyl 8-(6-(((2,4-dimethoxybenzyl)amino)methyl)-2-(pyridin-4-yl)pyrido[3,4-d]pyrimidin-4-yl)-2,8-diazaspiro[4.5]decane-2-carboxylate

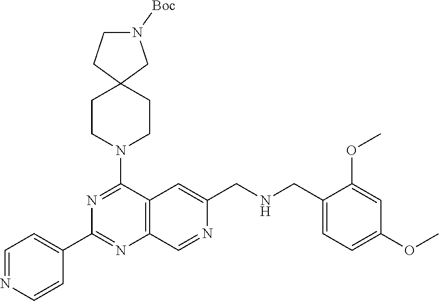 [SMILES: COc1ccc(CNCc2cc3c(N4CCC5(CCN(C(=O)OC(C)(C)C)C5)CC4)nc(-c4ccncc4)nc3cn2)c(OC)c1]

To a solution of tert-butyl 8-[6-formyl-2-(4-pyridyl)pyrido[3,4-d]pyrimidin-4-yl]-2,8-diazaspiro[4.5]decane-2-carboxylate (80 mg, 0.17 mmol) in dichloromethane (4 mL) was added 2,4-dimethoxybenzylamine (30 uL, 0.2 mmol) and a drop of acetic acid. Sodium triacetoxyborohydride (106 mg, 0.51 mmol) was added and the reaction mixture was stirred at room temperature for 1 hour. After complete conversion to the amine, a saturated solution of sodium bicarbonate (15 mL) was added to reaction mixture and it was extracted 3 times with ethyl acetate (3×20 mL). Organics were combined, dried with anhydrous sodium sulfate, filtered and concentrated to dryness. The compound was used directly for the next step.

### Step 7: tert-butyl 8-(6-((N-(2,4-dimethoxybenzyl)propionamido)methyl)-2-(pyri din-4-yl)pyrido[3,4-d]pyrimidin-4-yl)-2,8-diazaspiro[4.5]decane-2-carboxylate

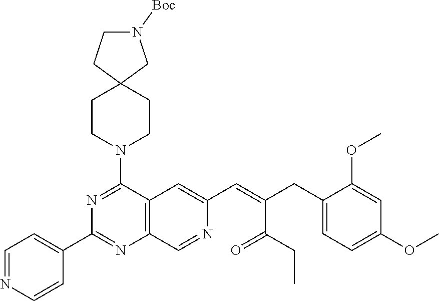 [SMILES: CCC(=O)/C(=C\c1cc2c(N3CCC4(CCN(C(=O)OC(C)(C)C)C4)CC3)nc(-c3ccncc3)nc2cn1)Cc1ccc(OC)cc1OC]

The crude residue from Step 4 was diluted back in dichloromethane (4 mL) with triethylamine (70 uL, 0.51 mmol) and propionyl chloride (20 uL, 0.19 mmol) was added to the mixture. The reaction mixture was stirred at room temperature for 30 minutes and was concentrated to dryness and directly loaded on a silica gel chromatography column, eluted from 1 to 12% MeOH in DCM to provide the title compound tert-butyl 8-[6-[[(2,4-dimethoxyphenyl)methyl-propanoyl-amino]methyl]-2-(4-pyridyl)pyrido[3,4-d]pyrimidin-4-yl]-2,8-diazaspiro[4.5]decane-2-carboxylate (62 mg, 54% yield) as a beige solid. LCMS (ESI) m/z: 682.6 [M+H]⁺.

### Step 8: N-((2-(pyridin-4-yl)-4-(2,8-diazaspiro[4.5]decan-8-yl)pyrido[3,4-d]pyrimidin-6-yl)methyl)propionamide; formic acid salt

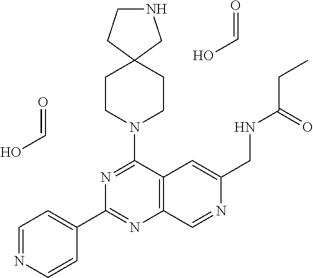 [SMILES: O=CO ; CCC(=O)NCc1cc2c(N3CCC4(CCNC4)CC3)nc(-c3ccncc3)nc2cn1 ; O=CO]

To a solution of tert-butyl 8-[6-[[(2,4-dimethoxyphenyl)methyl-propanoyl-amino]methyl]-2-(4-pyridyl)pyrido[3,4-d]pyrimidin-4-yl]-2,8-diazaspiro[4.5]decane-2-carboxylate (39 mg, 0.06 mmol) in dichloromethane (4 mL) was added HBr in acetic acid (0.3 mL, 1.45 mmol) and the reaction mixture was stirred at room temperature 2 hours after which the reaction mixture was concentrated under reduced pressure. The residual solid was directly loaded in water on top of a C18 column and purified by reverse phase column chromatography (MeCN/Aqueous 10 mM Ammonium Formate buffered at pH=3.8). Pure fractions were directly lyophilized to provide the title compound N-[[4-(2,8-diazaspiro[4.5]decan-8-yl)-2-(4-pyridyl)pyrido[3,4-d]pyrimidin-6-yl]methyl]propanamide; formic acid salt (16 mg, 54% yield) as an off white solid. LCMS (ESI) m/z: 432.3 [M+H]⁺. ¹H NMR (400 MHz, DMSO-d₆) δ 9.21 (s, 1H), 8.74 (dd, J=4.6, 1.4 Hz, 2H), 8.55 (t, J=5.7 Hz, 1H), 8.37 (s, 2H), 8.30 (dd, J=4.5, 1.4 Hz, 2H), 7.65 (s, 1H), 4.50 (d, J=5.8 Hz, 2H), 3.96-3.80 (m, 4H), 3.19 (t, J=7.3 Hz, 2H), 3.02 (s, 2H), 2.26-2.17 (m, 2H), 1.84 (t, J=7.3 Hz, 2H), 1.81-1.65 (m, 4H), 1.05 (t, J=7.6 Hz, 3H).

### Example 160

### 6-methyl-2-(3-methyl-1H-pyrazol-4-yl)-4-(2,8-diazaspiro[4.5]decan-8-yl)pyrido[3,4-d]pyrimidine (Compound 160) formate

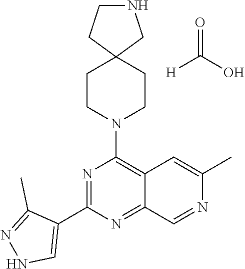

### Step 1: 6-chloro-2-(3-methyl-1-((2-(trimethylsilyl)ethoxy)methyl)-1H-pyrazol-4-yl)pyrido[3,4-d]pyrimidin-4-ol

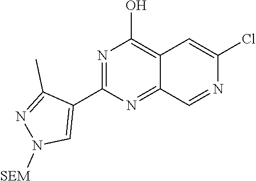 [SMILES: Cc1nn(COCC[Si](C)(C)C)cc1-c1nc(O)c2cc(Cl)ncc2n1]

Following the procedure described in Example 142, step 2 and making non-critical variations as required to replace 3-aminopyridine-4-carboxamide with 5-amino-2-chloroisonicotinamide (prepared according to the procedure in US2019270742), the title compound was obtained as an off-white solid. ¹H NMR (400 MHz, DMSO-d₆) δ 12.59 (s, 1H), 8.84-8.78 (m, 1H), 8.69-8.28 (m, 1H), 7.91 (s, 1H), 5.54-5.35 (m, 2H), 3.61-3.51 (m, 2H), 2.78-2.52 (m, 3H), 0.91-0.79 (m, 2H), 0.01-0.09 (m, 9H). LCMS (ESI) m/z: 392.0 [M+H]⁺.

### Step 2: 4,6-dichloro-2-(3-methyl-1-((2-(trimethylsilyl)ethoxy)methyl)-1H-pyrazol-4-yl)pyrido[3,4-d]pyrimidine

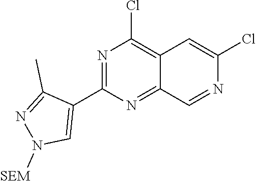 [SMILES: Cc1nn(COCC[Si](C)(C)C)cc1-c1nc(Cl)c2cc(Cl)ncc2n1]

To a solution of 6-chloro-2-(3-methyl-1-((2-(trimethylsilyl)ethoxy)methyl)-1H-pyrazol-4-yl)pyrido[3,4-d]pyrimidin-4-ol (2 g, 5.10 mmol) in phosphorus oxychloride (14 mL) was added N,N-diisopropylethylamine (0.89 mL, 5.1 mmol). The reaction mixture was stirred at room temperature for 16 hours and concentrated in vacuo. The crude residue was dissolved in DCM (150 mL) and basified with sat. aq. NaHCO₃ (50 mL) to pH 8 at 0° C. The organic layer was dried over anhydrous Na₂SO₄, filtered and concentrated in vacuo. The crude residue was purified by silica gel chromatography (solvent gradient: 0-30% EtOAc in petroleum ether) to give the title compound (620 mg, 30%) as a yellow solid. LCMS (ESI) m/z: 410.4 [M+H]⁺.

### Step 3: tert-butyl 8-(6-chloro-2-(3-methyl-1-((2-(trimethylsilyl)ethoxy)methyl)-1H-pyrazol-4-yl)pyrido[3,4-d]pyrimidin-4-yl)-2,8-diazaspiro[4.5]decane-2-carboxylate

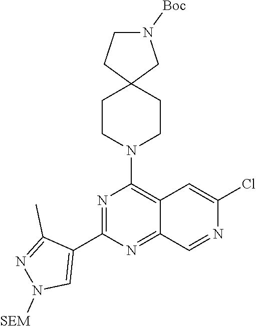 [SMILES: Cc1nn(COCC[Si](C)(C)C)cc1-c1nc(N2CCC3(CCN(C(=O)OC(C)(C)C)C3)CC2)c2cc(Cl)ncc2n1]

Following the procedure described in Example 101 and making non-critical variations as required to replace 4-chloro-2-(pyridin-4-yl)pyrido[3,4-d]pyrimidine with 4,6-dichloro-2-(3-methyl-1-((2-(trimethylsilyl)ethoxy)methyl)-1H-pyrazol-4-yl)pyrido[3,4-d]pyrimidine, the title compound was obtained as a yellow solid. LCMS (ESI) m/z: 614.2 [M+H]⁺.

### Step 4: tert-butyl 8-(6-methyl-2-(3-methyl-1-((2-(trimethylsilyl)ethoxy)methyl)-1H-pyrazol-4-yl)pyrido[3,4-d]pyrimidin-4-yl)-2,8-diazaspiro[4.5]decane-2-carboxylate

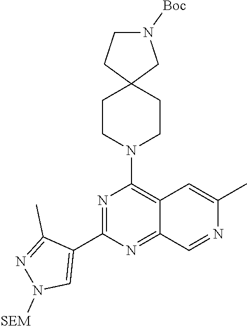 [SMILES: Cc1cc2c(N3CCC4(CCN(C(=O)OC(C)(C)C)C4)CC3)nc(-c3cn(COCC[Si](C)(C)C)nc3C)nc2cn1]

Following the procedure described in Example 158 and making non-critical variations as required to replace 6-chloro-2-(pyridin-4-yl)-4-((2-(trimethylsilyl)ethoxy)methoxy) pyrido[3,4-d]pyrimidine with tert-butyl 8-(6-chloro-2-(3-methyl-1-((2-(trimethylsilyl)ethoxy)methyl)-1H-pyrazol-4-yl)pyrido[3,4-d]pyrimidin-4-yl)-2,8-diazaspiro[4.5]decane-2-carboxylate, the title compound was obtained as a yellow solid. LCMS (ESI) m/z: 594.2 [M+H]⁺.

### Step 5: 6-methyl-2-(3-methyl-1H-pyrazol-4-yl)-4-(2,8-diazaspiro[4.5]decan-8-yl)pyrido[3,4-d]pyrimidine formate

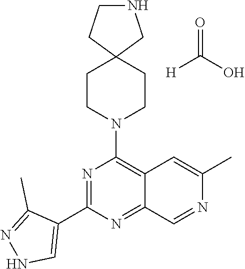

To a solution of tert-butyl 8-(6-methyl-2-(3-methyl-1-((2-(trimethylsilyl)ethoxy)methyl)-1H-pyrazol-4-yl)pyrido[3,4-d]pyrimidin-4-yl)-2,8-diazaspiro[4.5]decane-2-carboxylate (60 mg, 0.1 mmol) in EtOAc (1 mL) was added 4M HCl in EtOAc (1 mL, 4.0 mmol). The mixture was stirred at room temperature for 5 hours and concentrated in vacuo. The resulting residue was purified by reverse phase chromatography (acetonitrile 1-30%/0.225% formic acid in water) to give the title compound (6 mg, 16%) as a white solid. ¹H NMR (400 MHz, DMSO-d₆) δ 9.00 (s, 1H), 8.40 (s, 1H), 8.13 (s, 1H), 7.58 (s, 1H), 3.83-3.72 (m, 4H), 3.23-3.17 (m, 2H), 3.02 (s, 2H), 2.65 (s, 3H), 2.61 (s, 3H), 1.98-1.62 (m, 6H). LCMS (ESI) m/z: 364.1 [M+H]⁺.

### Example 161

### 5-chloro-2-(pyridin-4-yl)-4-(2,8-diazaspiro[4.5]decan-8-yl)pyrido[3,4-d]pyrimidine (Compound 161)

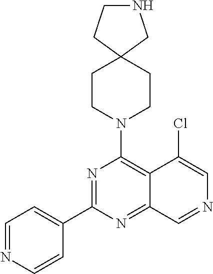 [SMILES: Clc1cncc2nc(-c3ccncc3)nc(N3CCC4(CCNC4)CC3)c12]

### Step 1: 3,5-dichloro-N-(imino(pyridin-4-yl)methyl)isonicotinamide

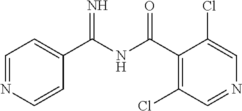 [SMILES: N=C(NC(=O)c1c(Cl)cncc1Cl)c1ccncc1]

2,6-dichloroisonicotinic acid (1309 mg, 6.82 mmol) and HATU (2852 mg, 7.5 mmol) were dissolved in DMF (10 mL). To this solution was added DIEA (3.56 mL, 20.5 mmol), and the mixture was stirred at room temperature for 10 minutes. Then pyridine-4-carboxamidine hydrochloride (1289 mg, 8.18 mmol) was added to the reaction mixture. After 3 hours, to the reaction mixture was added a saturated aqueous solution of NaHCO₃ (20 mL). It was then extracted with EtOAc (2×100 mL) and 10% MeOH/DCM (100 mL). The combined organic layers were dried over Na₂SO₄, filtered and concentrated to dryness to provide the title compound 3,5-dichloro-N-(imino(pyridin-4-yl)methyl)isonicotinamide (765 mg, yield 38%) as a yellow solid. LCMS (ESI) m/z: 295.1, [M+H].

### Step 2: 5-chloro-2-(pyridin-4-yl)pyrido[3,4-d]pyrimidin-4-ol

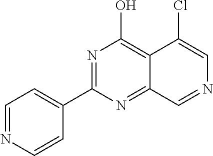 [SMILES: Oc1nc(-c2ccncc2)nc2cncc(Cl)c12]

3,5-dichloro-N-(pyridine-4-carboximidoyl)pyridine-4-carboxamide (740 mg, 2.51 mmol) was suspended in DMA (6 mL) in a microwave vial. To this was then added K₂CO₃ (347 mg, 2.51 mmol), DIEA (0.44 mL, 2.51 mmol), and DBU (0.37 mL, 2.51 mmol). The vial was sealed and irradiated at 150° C. for 45 minutes in a microwave reactor. The reaction mixture was concentrated under an air stream then re-dissolved in DMF and purified by C18 reverse phase chromatography (MeCN/Aqueous 10 mM Ammonium Formate buffered at pH=3.8). Fraction containing product were combined and lyophilized to yield the title compound 5-chloro-2-(pyridin-4-yl)pyrido[3,4-d]pyrimidin-4-ol was obtained as a yellow solid. LCMS (ESI) m/z: 259.2, [M+H]⁺.

### Step 3: tert-butyl 8-(5-chloro-2-(pyridin-4-yl)pyrido[3,4-d]pyrimidin-4-yl)-2,8-diazaspiro[4.5]decane-2-carboxylate

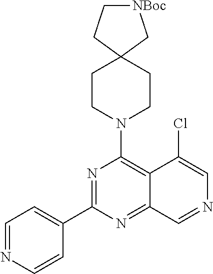 [SMILES: CC(C)(C)OC(=O)N1CCC2(CCN(c3nc(-c4ccncc4)nc4cncc(Cl)c34)CC2)C1]

Following the procedure described in Example 159, Step 3 and making non-critical variations as required to replace 6-bromo-2-(4-pyridyl)pyrido[3,4-d]pyrimidin-4-ol with 5-chloro-2-(pyridin-4-yl)pyrido[3,4-d]pyrimidin-4-ol the title compound was obtained (5 mg, 22% yield) as a yellow waxy solid. LCMS (ESI) m/z: 481.1, [M+H]⁺.

### Step 4: 5-chloro-2-(pyridin-4-yl)-4-(2,8-diazaspiro[4.5]decan-8-yl)pyrido[3,4-d]pyrimidine

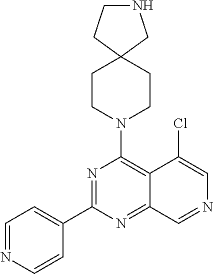 [SMILES: Clc1cncc2nc(-c3ccncc3)nc(N3CCC4(CCNC4)CC3)c12]

tert-butyl 8-[5-chloro-2-(4-pyridyl)pyrido[3,4-d]pyrimidin-4-yl]-2,8-diazaspiro[4.5]decane-2-carboxylate (5.0 mg, 0.0100 mmol) was dissolved in EtOAc (2 mL) and treated with 4N HCl in dioxane (0.5 mL) and stirred at room temperature. After 1 h, the mixture was concentrated to dryness and gave a solid residue. The residue was triturated with MeCN (3 mL) and concentrated to dryness again (repeated×2). The resulting residue was dissolved in a mixture of water and MeCN and lyophilized to provide the title compound 5-chloro-2-(pyridin-4-yl)-4-(2,8-diazaspiro[4.5]decan-8-yl)pyrido[3,4-d]pyrimidine hydrochloride (4.5 mg, quantitative yield) as a yellow solid. LCMS (ESI) m/z: 381.1, [M+H]⁺. ¹H NMR (400 MHz, DMSO-d₆) δ 9.21 (s, 1H), 9.03 (br, 1H), 8.90-8.83 (m, 2H), 8.67 (s, 1H), 8.49-8.39 (m, 2H), 3.86-3.76 (m, 4H), 3.29-3.21 (m, 2H), 3.17-2.95 (m, 2H), 1.89-1.60 (m, 6H).

### Example 162

### 5-methyl-2-(pyridin-4-yl)-4-(2,8-diazaspiro[4.5]decan-8-yl)pyrido[3,4-d]pyrimidine (Compound 162)

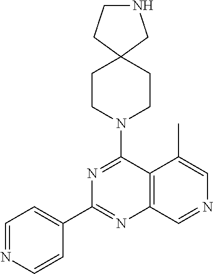 [SMILES: Cc1cncc2nc(-c3ccncc3)nc(N3CCC4(CCNC4)CC3)c12]

### Step 1: 3-bromo-5-fluoro-N-(imino(pyridin-4-yl)methyl)isonicotinamide

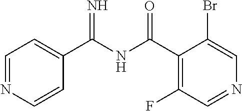 [SMILES: N=C(NC(=O)c1c(F)cncc1Br)c1ccncc1]

Pyridine-4-carboxamidine hydrochloride (1.38 g, 8.75 mmol) and 3-bromo-5-fluoro-pyridine-4-carboxylic acid (2.0 g, 9.1 mmol) were dissolved in DMF (45 mL) with diisopropylethylamine (4.75 mL, 27 mmol). Finally, HATU (3.63 g, 9.55 mmol) was added and the reaction mixture was stirred at room temperature for 16 hours. A saturated solution of sodium bicarbonate (80 mL) was added to reaction mixture and it was extracted 3 times with a 2:8 mixture of iPrOH—CHCl₃ (3×50 mL). Organics were combined, thoroughly washed with water, brine, dried with anhydrous sodium sulfate, filtered and concentrated to dryness. The crude residue was purified by trituration in MeOH. The off white precipitates was filtered, rinsed with MeOH and dried to provide the title compound 3-bromo-5-fluoro-N-(imino(pyridin-4-yl)methyl)isonicotinamide (1.93 g, 66% yield) as a beige solid. LCMS (ESI) m/z: 323.0/325.0 (Br pattern) [M+H].

### Step 2: 5-bromo-2-(pyridin-4-yl)pyrido[3,4-d]pyrimidin-4 (3H)-one

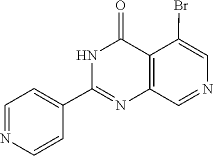 [SMILES: O=c1[nH]c(-c2ccncc2)nc2cncc(Br)c12]

3-bromo-5-fluoro-N-(imino(pyridin-4-yl)methyl)isonicotinamide (1.93 g, 5.96 mmol) was dissolved in DMF (15 mL) and cesium carbonate (3.9 g, 11.9 mmol) was added. The reaction mixture was stirred at 100° C. for 2 hours. Once conversion completed, the reaction mixture was cooled to room temperature and added dropwise to a stirring solution of NH₄Cl (sat) diluted 1:1 with water (150 mL total). An off white precipitate formed and was filtered and rinsed with water and acetonitrile. The solid was dried to provide the title compound 5-bromo-2-(pyridin-4-yl)pyrido[3,4-d]pyrimidin-4 (3H)-one (1.72 g, 95% yield) as an off white solid. LCMS (ESI) m/z: 302.9/304.9 (Br pattern) [M+H]⁺.

### Step 3: 5-methyl-2-(pyridin-4-yl)pyrido[3,4-d]pyrimidin-4 (3H)-one

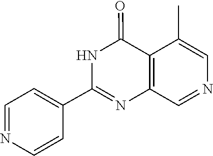 [SMILES: Cc1cncc2nc(-c3ccncc3)[nH]c(=O)c12]

A microwave vial was charged with 5-bromo-2-(pyridin-4-yl)pyrido[3,4-d]pyrimidin-4 (3H)-one (400 mg, 1.32 mmol), methylboronic acid (180 mg, 3.0 mmol), Pd(PPh₃)₄ (154 mg, 0.13 mmol), and K₂CO₃ (548 mg, 3.96 mmol). The vial was capped and DMA (6 mL) was added and N₂ sparged 5 min then irradiated at 150° C. for 1 hour in the microwave reactor. Volatiles were removed under an air stream. Crude residue suspended in MeOH (30 mL) and EtOAc (30 mL) and 5 g of silica gel added and concentrated in vacuo to dry-load the material. Purification by column chromatography (MeOH/EtOAc/Heptanes) to provide the title compound 5-methyl-2-(pyridin-4-yl)pyrido[3,4-d]pyrimidin-4 (3H)-one (26 mg, 7% yield) as a yellow solid. LCMS (ESI) m/z: 239.2 [M+H]⁺.

### Step 4: tert-butyl 8-(5-methyl-2-(pyridin-4-yl)pyrido[3,4-d]pyrimidin-4-yl)-2,8-diazaspiro[4.5]decane-2-carboxylate

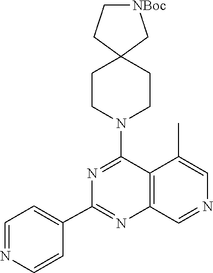 [SMILES: Cc1cncc2nc(-c3ccncc3)nc(N3CCC4(CCN(C(=O)OC(C)(C)C)C4)CC3)c12]

Following the procedure described in Example 159, Step 3 and making non-critical variations as required to replace 6-bromo-2-(4-pyridyl)pyrido[3,4-d]pyrimidin-4-ol with 5-methyl-2-(pyridin-4-yl)pyrido[3,4-d]pyrimidin-4 (3H)-one the title compound was obtained (16 mg, 26% yield) as a yellow solid. LCMS (ESI) m/z: 461.1 [M+H]⁺.

### Step 5: 5-methyl-2-(pyridin-4-yl)-4-(2,8-diazaspiro[4.5]decan-8-yl)pyrido[3,4-d]pyrimidine; formic acid salt

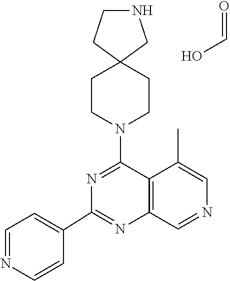 [SMILES: Cc1cncc2nc(-c3ccncc3)nc(N3CCC4(CCNC4)CC3)c12 ; O=CO]

Following the procedure described for Example 159, Step 6 starting from 16 mg, 0.04 mmol of tert-butyl 8-(5-methyl-2-(pyridin-4-yl)pyrido[3,4-d]pyrimidin-4-yl)-2,8-diazaspiro[4.5]decane-2-carboxylate to provide 5-methyl-2-(pyridin-4-yl)-4-(2,8-diazaspiro[4.5]decan-8-yl)pyrido[3,4-d]pyrimidine; formic acid salt (6 mg, 59% yield) as a beige solid. ¹H NMR (400 MHz, CD₃OD) δ 9.10 (s, 1H), 8.72 (d, J=5.9 Hz, 2H), 8.54 (bs, 1H), 8.47 (dd, J=4.6, 1.6 Hz, 2H), 8.43 (s, 1H), 3.91-3.68 (m, 4H), 3.41 (t, J=7.4 Hz, 2H), 3.21-3.05 (m, 2H), 2.77 (s, 3H), 2.20-1.94 (m, 2H), 1.92-1.74 (m, 4H). LCMS (ESI) m/z: 361.1 [M+H]⁺.

### Example 163

### 8-methyl-2-(3-methyl-1H-pyrazol-4-yl)-4-(2,8-diazaspiro[4.5]decan-8-yl)pyrido[3,4-d]pyrimidine (Compound 163)

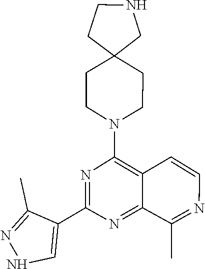 [SMILES: Cc1n[nH]cc1-c1nc(N2CCC3(CCNC3)CC2)c2ccnc(C)c2n1]

Following the procedure described in Example 142 and making non-critical variations as required to replace 3-aminopyridine-4-carboxamide with 3-amino-2-methylisonicotinamide (prepared according to the procedure in Synthesis, 2016, 48, 1226), the title compound was obtained as a yellow solid. ¹H NMR (400 MHz, DMSO-d₆) δ 8.34 (s, 1H), 8.30 (d, J=5.6 Hz, 1H), 8.17 (s, 1H), 7.58 (d, J=5.6 Hz, 1H), 3.84-3.76 (m, 4H), 3.13 (t, J=7.2 Hz, 2H), 2.94 (s, 2H), 2.85 (s, 3H), 2.68 (s, 3H), 1.80 (t, J=7.6 Hz, 2H), 1.76-1.71 (m, 4H). LCMS (ESI) m/z: 364.1 [M+H]⁺.

### Example 164

### 8-(2-(pyridin-4-yl)pyrido[3,4-d]pyrimidin-4-yl)-2,8-diazaspiro[4.5]decan-3-one (Compound 164)

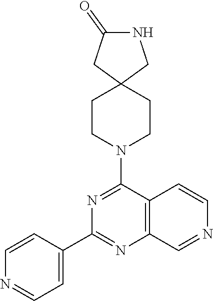 [SMILES: O=C1CC2(CCN(c3nc(-c4ccncc4)nc4cnccc34)CC2)CN1]

Following the procedure described in Example 101, Step 3 and making non-critical variations as required to replace tert-butyl 2,8-diazaspiro[4.5]decane-2-carboxylate with 2,8-diazaspiro[4.5]decan-3-one the title compound was obtained. ¹H NMR (400 MHz, DMSO-d₆) δ 9.27 (d, J=0.8 Hz, 1H), 8.80-8.75 (m, 2H), 8.60 (d, J=5.7 Hz, 1H), 8.36-8.31 (m, 2H), 7.90 (dd, J=5.8, 0.9 Hz, 1H), 7.60 (s, 1H), 4.12-4.01 (m, 2H), 3.94-3.84 (m, 2H), 3.17 (s, 2H), 2.21 (s, 2H), 1.87-1.75 (m, 4H). LCMS (ESI) m/z: 361.1 [M+H].

### Example 165

### 8-(2-(pyridin-4-yl)pyrido[3,4-d]pyrimidin-4-yl)-2,8-diazaspiro[4.5]decan-1-one (Compound 165)

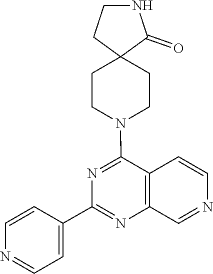 [SMILES: O=C1NCCC12CCN(c1nc(-c3ccncc3)nc3cnccc13)CC2]

Following the procedure described in Example 101, Step 3 and making non-critical variations as required to replace tert-butyl 2,8-diazaspiro[4.5]decane-2-carboxylate with 2,8-diazaspiro[4.5]decan-1-one hydrochloride the title compound was obtained as a solid. ¹H NMR (400 MHz, DMSO-d₆) δ 9.28 (d, J=0.8 Hz, 1H), 8.80-8.75 (m, 2H), 8.60 (d, J=5.7 Hz, 1H), 8.36-8.31 (m, 2H), 7.93 (dd, J=5.7, 0.9 Hz, 1H), 7.67 (s, 1H), 4.52-4.42 (m, 2H), 3.67-3.57 (m, 2H), 3.28-3.21 (m, 2H), 2.14-2.07 (m, 2H), 1.99-1.88 (m, 2H), 1.67-1.57 (m, 2H). LCMS (ESI) m/z: 361.01 [M+H]⁺.

### Example 166

### 4-(1-methyl-2,8-diazaspiro[4.5]decan-8-yl)-2-(pyridin-4-yl)pyrido[3,4-d]pyrimidine (Compound 166)

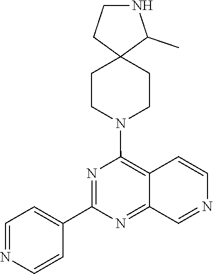 [SMILES: CC1NCCC12CCN(c1nc(-c3ccncc3)nc3cnccc13)CC2]

### Step 1: benzyl 1-methyl-2,8-diazaspiro[4.5]decane-2-carboxylate trifluoro acetate

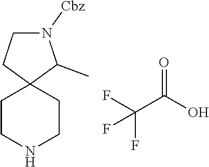

To a solution of 2-benzyl 8-tert-butyl 1-methyl-2,8-diazaspiro[4.5]decane-2,8-dicarboxylate (200 mg, 0.51 mmol) (prepared according to the procedure in J. Org. Chem., 2016, 81, 3509) in DCM (4 mL) was added trifluoroacetic acid (2 mL, 2.69 mmol). The mixture was stirred at room temperature for 1 hour. The mixture was concentrated in vacuo to give the title compound (200 mg, crude) as yellow oil that required no further purification. LCMS (ESI) m/z: 289.3 [M+H]⁺.

### Step 2: benzyl 1-methyl-8-(2-(pyridin-4-yl)pyrido[3,4-d]pyrimidin-4-yl)-2,8-diazaspiro[4.5]decane-2-carboxylate

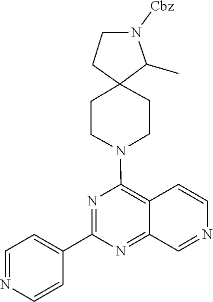 [SMILES: CC1N(C(=O)OCc2ccccc2)CCC12CCN(c1nc(-c3ccncc3)nc3cnccc13)CC2]

Following the procedure described in Example 102 and making non-critical variations as required to replace tert-butyl 2,8-diazaspiro[4.5]decane-2-carboxylate with benzyl 1-methyl-2,8-diazaspiro[4.5]decane-2-carboxylate trifluoroacetate, the title compound was obtained as a yellow solid. LCMS (ESI) m/z: 495.6 [M+H]⁺.

### Step 3: 4-(1-methyl-2,8-diazaspiro[4.5]decan-8-yl)-2-(pyridin-4-yl)pyrido[3,4-d]pyrimidine

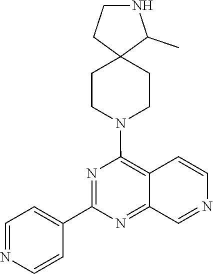 [SMILES: CC1NCCC12CCN(c1nc(-c3ccncc3)nc3cnccc13)CC2]

To a solution of benzyl 1-methyl-8-(2-(pyridin-4-yl)pyrido[3,4-d]pyrimidin-4-yl)-2,8-diazaspiro[4.5]decane-2-carboxylate (60 mg, 0.12 mmol) in AcOH (1 mL) was added HBr (1 mL, 33% in AcOH). The mixture was stirred at room temperature for 16 hours. The mixture was concentrated in vacuo. The residue was purified by reverse phase chromatography (acetonitrile 6
36%/0.225% formic acid in water) to give the title compound (18 mg, 41%) as a white solid.

¹H NMR (400 MHz, DMSO-d₆) δ 9.27 (s, 1H), 8.77 (d, J=4.8 Hz, 2H), 8.60 (d, J=5.2 Hz, 1H), 8.35-8.32 (m, 3H), 7.92 (d, J=5.2 Hz, 1H), 4.53-4.45 (m, 2H), 3.54-3.42 (m, 3H), 3.23-3.16 (m, 2H), 2.25-2.19 (m, 1H), 1.92-1.72 (m, 2H), 1.70-1.68 (m, 1H), 1.66-1.54 (m, 2H), 1.21-1.13 (m, 3H). LCMS (ESI) m/z: 361.1 [M+H]⁺.

### Example 167

### 4-(3-methyl-2,8-diazaspiro[4.5]decan-8-yl)-2-(pyridin-4-yl)pyrido[3,4-d]pyrimidine (Compound 167)

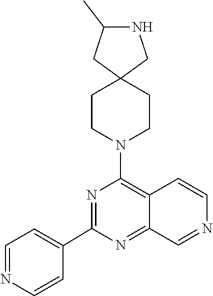 [SMILES: CC1CC2(CCN(c3nc(-c4ccncc4)nc4cnccc34)CC2)CN1]

Following the procedure described in Example 166 and making non-critical variations as required to replace 2-benzyl 8-tert-butyl 1-methyl-2,8-diazaspiro[4.5]decane-2,8-dicarboxylate with 2-benzyl 8-tert-butyl 3-methyl-2,8-diazaspiro[4.5]decane-2,8-dicarboxylate (prepared according to the procedure in J. Org. Chem. 2016, 81, 3509), the title compound was obtained as a yellow solid. ¹H NMR (400 MHz, DMSO-d₆) δ 9.27 (s, 1H), 8.79-8.75 (m, 2H), 8.60 (d, J=5.6 Hz, 1H), 8.36 (s, 1H), 8.34-8.32 (m, 2H), 7.90 (d, J=5.6 Hz, 1H), 4.01-3.90 (m, 4H), 3.59-3.54 (m, 1H), 3.10-2.96 (m, 2H), 2.17-2.10 (m, 1H), 1.84-1.76 (m, 4H), 1.44-1.35 (m, 1H), 1.27 (d, J=6.4 Hz, 3H). LCMS (ESI) m/z: 361.4 [M+H]⁺.

### Example 168

### 4-(2,3-dimethyl-2,8-diazaspiro[4.5]decan-8-yl)-2-(pyridin-4-yl)pyrido[3,4-d]pyrimidine (Compound 168)

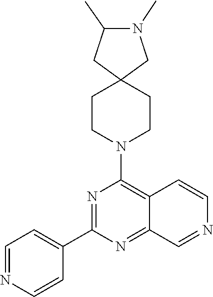 [SMILES: CC1CC2(CCN(c3nc(-c4ccncc4)nc4cnccc34)CC2)CN1C]

Following the procedure described in Example 102 and making non-critical variations as required to replace 4-(2,8-diazaspiro[4.5]decan-8-yl)-2-(4-pyridyl)pyrido[3,4-d]pyrimidine hydrochloride with 4-(3-methyl-2,8-diazaspiro[4.5]decan-8-yl)-2-(pyridin-4-yl)pyrido[3,4-d]pyrimidine, the title compound was obtained as a yellow solid. ¹H NMR (400 MHz, DMSO-d₆) δ 9.25 (s, 1H), 8.76 (d, J=5.2 Hz, 2H), 8.58 (d, J=5.6 Hz, 1H), 8.31 (d, J=5.6 Hz, 2H), 7.87 (d, J=5.6 Hz, 1H), 4.06-3.78 (m, 5H), 3.47-3.43 (m, 1H), 3.12-3.08 (m, 1H), 2.29 (s, 3H), 1.99-1.93 (m, 1H), 1.81-1.66 (m, 4H), 1.38-1.32 (m, 1H), 1.11 (d, J=5.6 Hz, 3H). LCMS (ESI) m/z: 375.1 [M+H]⁺.

### Examples 169 and 170

### (1S,3R)-3-(8-(2-(pyridin-4-yl)pyrido[3,4-d]pyrimidin-4-yl)-2,8-diazaspiro[4.5]decan-2-yl)cyclopentanol and (1R,3S)-3-(8-(2-(pyridin-4-yl)pyrido[3,4-d]pyrimidin-4-yl)-2,8-diazaspiro[4.5]decan-2-yl)cyclopentanol (Compounds 169 and 170) formate

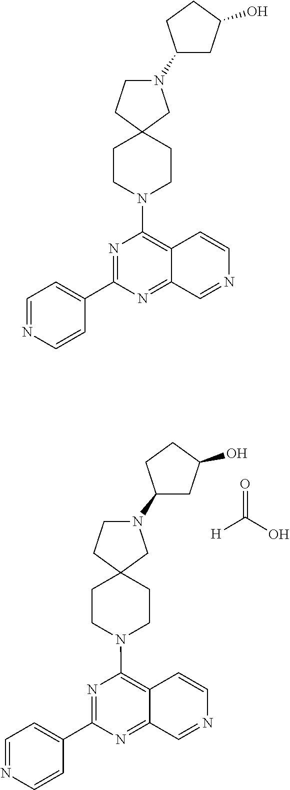

### Step 1: trans-3-((tert-butyldimethylsilyl)oxy)cyclopentanol

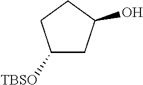 [SMILES: CC(C)(C)[Si](C)(C)O[C@@H]1CC[C@@H](O)C1]

To a solution of 1,3-cyclopentanediol (2.3 mL, 24.5 mmol, trans:cis=2:1) and imidazole (2.5 g, 37.0 mmol) in DCM (30 mL) was stirred for 10 minutes. Then TBSCl (3.7 g, 24.5 mmol) was added to this reaction mixture and stirred at room temperature for 16 hours. The mixture was diluted with DCM (50 mL), washed with water (30 mL) and brine (30 mL). The organic layer was dried over anhydrous Na₂SO₄, filtered and concentrated in vacuo. The crude residue was purified by silica gel chromatography (solvent gradient: 0-10% EtOAc in petroleum ether) to give the title compound (2.7 g, 51%) as yellow oil. ¹H NMR (400 MHz, CDCl₃) δ 4.49-4.35 (m, 2H), 2.13-1.97 (m, 2H), 1.84-1.77 (m, 2H), 1.61-1.45 (m, 2H), 0.87 (s, 9H), 0.04 (s, 6H).

### Step 2: trans-3-((tert-butyldimethylsilyl)oxy)cyclopentyl methanesulfonate

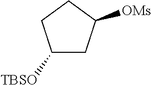 [SMILES: CC(C)(C)[Si](C)(C)O[C@@H]1CC[C@@H](OS(C)(=O)=O)C1]

To s solution of trans-3-((tert-butyldimethylsilyl)oxy)cyclopentanol (1.0 g, 4.62 mmol) and triethylamine (1.6 mL, 11.6 mmol) in DCM (10 mL) was added methanesulfonyl chloride (0.43 mL, 5.55 mmol). The solution was stirred at room temperature for 2 hours. The mixture was diluted with DCM (50 mL), washed with water (30 mL) and brine (30 mL). The organic layer was dried over anhydrous Na₂SO₄, filtered and concentrated in vacuo to give the title compound (1.1 g, crude) as yellow oil that required no further purification.

### Step 3: cis-4-(2-(3-((tert-butyldimethylsilyl)oxy)cyclopentyl)-2,8-diazaspiro[4.5]decan-8-yl)-2-(pyridin-4-yl)pyrido[3,4-d]pyrimidine

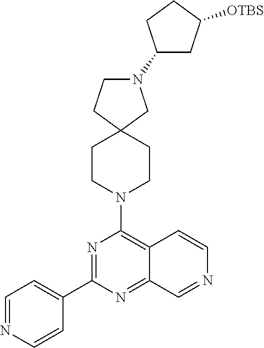 [SMILES: CC(C)(C)[Si](C)(C)O[C@H]1CC[C@@H](N2CCC3(CCN(c4nc(-c5ccncc5)nc5cnccc45)CC3)C2)C1]

To a solution of 4-(2,8-diazaspiro[4.5]decan-8-yl)-2-(4-pyridyl)pyrido[3,4-d]pyrimidine hydrochloride (500 mg, 1.31 mmol), trans-3-((tert-butyldimethylsilyl)oxy)cyclopentyl methanesulfonate (800 mg, 2.7 mmol) in DMF (10 mL) and acetonitrile (2 mL) was added K₂CO₃ (541 mg, 3.92 mmol). The mixture was heated to 90° C. and stirred for 16 hours. After cooling to room temperature, the mixture was diluted with EtOAc (100 mL), washed with water (50 mL×3) and brine (50 mL). The organic layer was dried over anhydrous Na₂SO₄, filtered and concentrated in vacuo. The crude residue was purified by silica gel chromatography (solvent gradient: 0-5% MeOH in DCM) to give the title compound (220 mg, 31%) as a yellow solid. LCMS (ESI) m/z: 545.1 [M+H]⁺.

### Step 4: cis-3-(8-(2-(pyridin-4-yl)pyrido[3,4-d]pyrimidin-4-yl)-2,8-diazaspiro[4.5]decan-2-yl)cyclopentanol

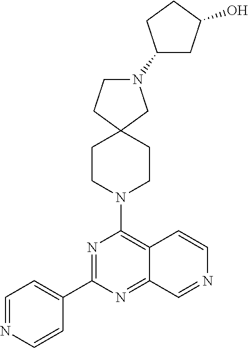 [SMILES: O[C@H]1CC[C@@H](N2CCC3(CCN(c4nc(-c5ccncc5)nc5cnccc45)CC3)C2)C1]

To a solution of cis-4-(2-(3-((tert-butyldimethylsilyl)oxy)cyclopentyl)-2,8-diazaspiro[4.5]decan-8-yl)-2-(pyridin-4-yl)pyrido[3,4-d]pyrimidine (220 mg, 0.4 mmol) in THF (10 mL) was added TBAF (2.4 mL, 2.4 mmol, 1M). The solution was stirred at room temperature for 16 hours. The mixture was concentrated in vacuo. The crude residue was purified by reverse phase chromatography (acetonitrile 5-35%/0.225% formic acid in water) to give the title compound (80 mg, 46%) as a yellow solid. LCMS (ESI) m/z: 431.2 [M+H]⁺.

### Step 5: (1S,3R)-3-(8-(2-(pyridin-4-yl)pyrido[3,4-d]pyrimidin-4-yl)-2,8-diazaspiro[4.5]decan-2-yl)cyclopentanol and (1R,3S)-3-(8-(2-(pyridin-4-yl)pyrido[3,4-d]pyrimidin-4-yl)-2,8-diazaspiro[4.5]decan-2-yl)cyclopentanol formate

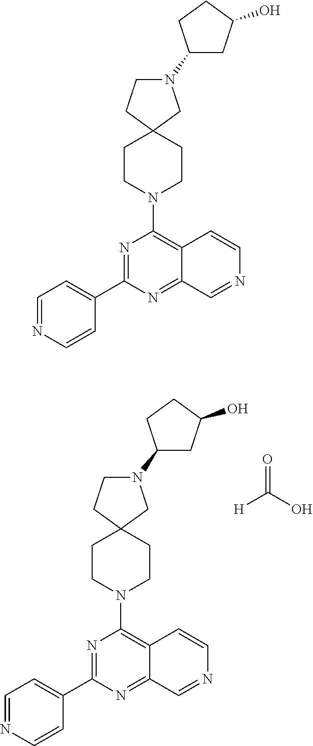 [SMILES: O[C@H]1CC[C@@H](N2CCC3(CCN(c4nc(-c5ccncc5)nc5cnccc45)CC3)C2)C1 ; O[C@@H]1CC[C@H](N2CCC3(CCN(c4nc(-c5ccncc5)nc5cnccc45)CC3)C2)C1 ; O=CO]

cis-3-(8-(2-(pyridin-4-yl)pyrido[3,4-d]pyrimidin-4-yl)-2,8-diazaspiro[4.5]decan-2-yl)cyclopentanol (80 mg, 0.19 mmol) was separated by using chiral SFC (Chiralpak-AD (250 mm*30 mm, 10 um), Supercritical CO₂/EtOH+0.1% NH₃·H₂O=50/50; 80 mL/min) to give the title compounds as white solids. The absolute configuration was arbitrarily assigned to each enantiomer. Example 169 (40 mg, first peak): ¹H NMR (400 MHz, DMSO-d₆) δ 9.25 (s, 1H), 8.76 (d, J=6.0 Hz, 2H), 8.58 (d, J=5.6 Hz, 1H), 8.31 (d, J=6.0 Hz, 2H), 7.87 (d, J=6.0 Hz, 1H), 4.05-3.95 (m, 3H), 3.89-3.83 (m, 2H), 2.68-2.65 (m, 2H), 2.58-2.52 (m, 3H), 2.11-2.00 (m, 1H), 1.81-1.65 (m, 9H), 1.55-1.36 (m, 2H). LCMS (ESI) m/z: 431.2 [M+H]⁺. Example 170 (33 mg, second peak): ¹H NMR (400 MHz, DMSO-d₆) δ 9.24 (s, 1H), 8.76 (d, J=5.6 Hz, 2H), 8.58 (d, J=5.6 Hz, 1H), 8.31 (d, J=5.6 Hz, 2H), 8.27 (s, 1H), 7.86 (d, J=6.0 Hz, 1H), 4.07-4.03 (m, 1H), 4.00-3.95 (m, 2H), 3.92-3.83 (m, 2H), 2.83-2.72 (m, 2H), 2.75-2.69 (m, 1H), 2.66 (s, 2H), 2.12-2.00 (m, 1H), 1.80-1.65 (m, 9H), 1.56-1.41 (m, 2H). LCMS (ESI) m/z: 431.2 [M+H]⁺.

### Example 171

### methyl 8-(2-(pyridin-4-yl)pyrido[3,4-d]pyrimidin-4-yl)-2,8-diazaspiro[4.5]decane-3-carboxylate (Compound 171)

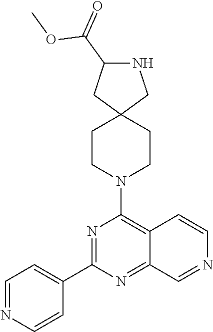 [SMILES: COC(=O)C1CC2(CCN(c3nc(-c4ccncc4)nc4cnccc34)CC2)CN1]

### Step 1: 2-(tert-butyl) 3-methyl 8-(2-(pyridin-4-yl)pyrido[3,4-d]pyrimidin-4-yl)-2,8-diazaspiro[4.5]decane-2,3-dicarboxylate

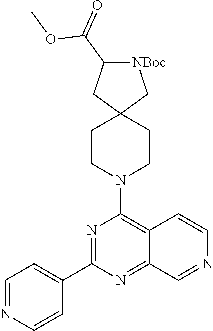 [SMILES: COC(=O)C1CC2(CCN(c3nc(-c4ccncc4)nc4cnccc34)CC2)CN1C(=O)OC(C)(C)C]

To a 20 mL vial was added 4-chloro-2-(4-pyridyl)pyrido[3,4-d]pyrimidine (770 mg, 3.2 mmol), potassium fluoride (550 mg, 9.5 mmol, 3 equiv.), and 2-tert-butyl 3-methyl 2,8-diazaspiro[4.5]decane-2,3-dicarboxylate hydrochloride (1100 mg, 3.2 mmol, 1 equiv.) were added to a 20 mL vial, followed by dimethyl sulfoxide (11 mL, 0.3 M) and triethylamine (2.2 mL, 16 mmol, 5 equiv.). The reaction was allowed to stir for 45 minutes at room temperature. The reaction mixture was transferred to a separatory funnel, diluted with water (10 mL), aqueous saturated NH₄Clsolution (10 mL) and EtOAc (20 mL) and the layers were separated. The aqueous layer was extracted with further EtOAc (3×15 mL) and the combined organic extracts were dried over Na₂SO₄, filtered, and concentrated in vacuo. The crude residue was further concentrated under reduced pressure on a Genevac for 16 hours to remove residual DMSO. The crude residue was then flashed on a 24 g Isco cartridge eluting 0 to 15% MeOH in DCM to furnish 2-(tert-butyl) 3-methyl 8-(2-(pyridin-4-yl)pyrido[3,4-d]pyrimidin-4-yl)-2,8-diazaspiro[4.5]decane-2,3-dicarboxylate (1.12 g, 70% yield). LCMS (ESI) m/z: 527.2 [M+Na]⁺.

### Step 2: methyl 8-(2-(pyridin-4-yl)pyrido[3,4-d]pyrimidin-4-yl)-2,8-diazaspiro[4.5]decane-3-carboxylate

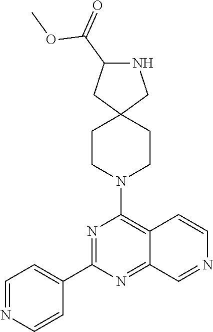 [SMILES: COC(=O)C1CC2(CCN(c3nc(-c4ccncc4)nc4cnccc34)CC2)CN1]

2-(tert-butyl) 3-methyl 8-(2-(pyridin-4-yl)pyrido[3,4-d]pyrimidin-4-yl)-2,8-diazaspiro[4.5]decane-2,3-dicarboxylate was dissolved in 1 mL DCM and 1 mL TFA. The mixture was stirred at room temperature for 30 minutes. The reaction mixture was then concentrated in vacuo, and then concentrated 2× further from DCM (5 mL) to remove residual TFA. The crude residue was then purified by HPLC to furnish methyl 8-(2-(pyridin-4-yl)pyrido[3,4-d]pyrimidin-4-yl)-2,8-diazaspiro[4.5]decane-3-carboxylate. ¹H NMR (400 MHz, DMSO-d₆) δ ¹H NMR (400 MHz, DMSO) δ 9.27 (d, J=3.3 Hz, 1H), 8.79-8.74 (m, 2H), 8.59 (dd, J=5.7, 2.6 Hz, 1H), 8.35-8.30 (m, 2H), 7.90 (d, J=5.7 Hz, 1H), 4.05-3.86 (m, 4H), 3.85-3.74 (m, 1H), 3.65 (s, 3H), 3.23-2.97 (m, 1H), 2.85-2.78 (m, 1H), 2.25-2.14 (m, 1H), 2.12-2.02 (m, 1H), 2.02-1.89 (m, 1H), 1.84-1.67 (m, 4H). LCMS (ESI) m/z: 405.2 [M+H]⁺.

### Example 172

### N-methyl-8-(2-(pyridin-4-yl)pyrido[3,4-d]pyrimidin-4-yl)-2,8-diazaspiro[4.5]decane-3-carboxamide (Compound 172)

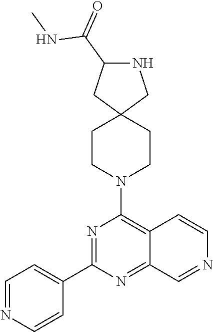 [SMILES: CNC(=O)C1CC2(CCN(c3nc(-c4ccncc4)nc4cnccc34)CC2)CN1]

### Step 1: 2-(tert-butoxycarbonyl)-8-(2-(pyridin-4-yl)pyrido[3,4-d]pyrimidin-4-yl)-2,8-diazaspiro[4.5]decane-3-carboxylic acid

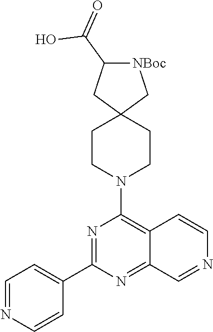 [SMILES: CC(C)(C)OC(=O)N1CC2(CCN(c3nc(-c4ccncc4)nc4cnccc34)CC2)CC1C(=O)O]

2-(tert-butyl) 3-methyl 8-(2-(pyridin-4-yl)pyrido[3,4-d]pyrimidin-4-yl)-2,8-diazaspiro[4.5]decane-2,3-dicarboxylate (1100 mg, 2.2 mmol) was added to a 20 mL vial followed by tetrahydrofuran (9700 mg, 11 mL, 130 mmol, 0.2 M), water (11000 mg, 11 mL, 610 mmol, 0.2 M), and lithium hydroxide (100 mg, 0.046 mL, 4.4 mmol, 2 equiv.). After 1 hour of stirring at room temperature, the mixture was diluted with DCM (10 mL), and the reaction was carefully quenched with aq. 1 N HCl; the aqueous layer was tested with pH paper to make sure it was neutral/acidic. The layers were separated, and the aqueous layer was extracted 1×15 mL DCM and, 8×15 mL of 80% CHCl3/20% IPA solution. The combined organics were dried over Na₂SO₄, filtered, and concentrated in vacuo. The crude residue was concentrated in vacuo 1x from PhMe (10 mL) which caused the product to solidify, giving the title compound (700 mg, 1.43 mmol, 700 mg, 65% Yield). LCMS (ESI) m/z: 547.3 [M+H]⁺.

### Step 2: N-methyl-8-(2-(pyridin-4-yl)pyrido[3,4-d]pyrimidin-4-yl)-2,8-diazaspiro[4.5]decane-3-carboxamide

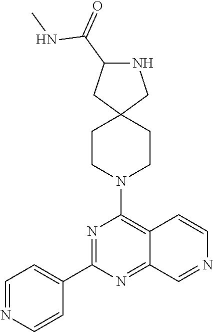 [SMILES: CNC(=O)C1CC2(CCN(c3nc(-c4ccncc4)nc4cnccc34)CC2)CN1]

To a 20 mL vial was added 2-(tert-butoxycarbonyl)-8-(2-(pyridin-4-yl)pyrido[3,4-d]pyrimidin-4-yl)-2,8-diazaspiro[4.5]decane-3-carboxylic acid (50 mg, 0.102 mmol, 1 equiv.), HATU (48 mg, 0.12 mmol, 1.2 equiv.) followed by DMF (1.1 mL), N,N-diisopropylethylamine (66 mg, 5 equiv), and Methylamine (2 M in THF solution, 0.11 mL, 2 equiv.). The reaction was allowed to stir at room temperature for 2 hours, and then was concentrated on the Genevac for 16 hours. The crude residue was then dissolved in DCM (5 mL) and water (5 mL). The layers were separated; the aqueous layer was extracted with DCM (4×5 mL). The combined organic extracts were dried over Na₂SO₄, filtered, and concentrated in vacuo. To this crude residue was added 0.5 mL DCM and 0.5 mL TFA and was allowed to stir at room temperature for 1 hour. The reaction mixture was then concentrated in vacuo, and then concentrated 2× further from DCM (5 mL) to remove as much residual TFA as possible. The crude residue was then purified by HPLC to fumish N-methyl-8-[2-(4-pyridyl)pyrido[3,4-d]pyrimidin-4-yl]-2,8-diazaspiro[4.5]decane-3-carboxamide (4.1 mg, 10% Yield). ¹H NMR (400 MHz, DMSO-d₆) δ 9.26 (s, 1H), 8.80-8.73 (m, 2H), 8.59 (d, J=5.7 Hz, 1H), 8.36-8.30 (m, 2H), 7.97-7.91 (m, 1H), 7.90 (d, J=5.7 Hz, 1H), 3.98 (t, J=5.6 Hz, 2H), 3.95-3.89 (m, 2H), 3.65 (t, J=8.1 Hz, 1H), 2.88 (d, J=10.8 Hz, 1H), 2.70 (d, J=10.8 Hz, 1H), 2.62 (d, J=4.8 Hz, 3H), 2.08 (dd, J=12.9, 8.7 Hz, 1H), 1.75-1.68 (m, 4H), 1.58 (dd, J=12.9, 7.5 Hz, 1H). Exchangeable NH amine proton was not observed. LCMS (ESI) m/z: 404.2 [M+H]⁺.

### Example 173

### N,N-dimethyl-8-(2-(pyridin-4-yl)pyrido[3,4-d]pyrimidin-4-yl)-2,8-diazaspiro[4.5]decane-3-carboxamide (Compound 173)

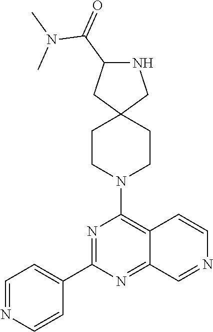 [SMILES: CN(C)C(=O)C1CC2(CCN(c3nc(-c4ccncc4)nc4cnccc34)CC2)CN1]

Following the procedure described in Example 172, Step 2 and making non-critical variations as required to replace methylamine with dimethylamine the title compound was obtained (4.11 mg, 10% Yield). ¹H NMR (400 MHz, DMSO-d₆) δ ¹H NMR (400 MHz, DMSO) δ 9.26 (s, 1H), 8.80-8.72 (m, 2H), 8.59 (d, J=5.8 Hz, 1H), 8.35-8.31 (m, 2H), 7.89 (d, J=5.7 Hz, 1H), 4.05-3.84 (m, 5H), 3.00 (s, 3H), 2.97 (d, J=11.0 Hz, 1H), 2.86 (s, 3H), 2.64 (d, J=11.1 Hz, 1H), 2.07 (dd, J=12.8, 8.9 Hz, 1H), 1.83-1.63 (m, 4H), 1.59 (dd, J=12.8, 6.9 Hz, 1H). Exchangeable NH amine proton not observed. LCMS (ESI) m/z: 418.2 [M+H]⁺.

### Example 174

### (8-(2-(pyridin-4-yl)pyrido[3,4-d]pyrimidin-4-yl)-2,8-diazaspiro[4.5]decan-3-yl)(pyrrolidin-1-yl)methanone (Compound 174)

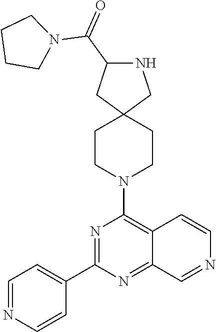 [SMILES: O=C(C1CC2(CCN(c3nc(-c4ccncc4)nc4cnccc34)CC2)CN1)N1CCCC1]

Following the procedure described in Example 172, Step 2 and making non-critical variations as required to replace methylamine with pyrrolidine the title compound was obtained (8.6 mg, 19% Yield). ¹H NMR (400 MHz, DMSO-d₆) δ 9.26 (s, 1H), 8.81-8.73 (m, 2H), 8.59 (d, J=5.6 Hz, 1H), 8.36-8.30 (m, 2H), 7.90 (d, J=5.7 Hz, 1H), 4.03-3.82 (m, 5H), 3.54 (dt, J=10.1, 6.6 Hz, 1H), 3.41-3.34 (m, 2H), 2.96 (d, J=11.0 Hz, 1H), 2.64 (d, J=11.0 Hz, 1H), 2.06 (dd, J=12.7, 8.7 Hz, 1H), 1.92-1.84 (m, 2H), 1.83-1.66 (m, 7H), 1.62 (dd, J=12.7, 6.9 Hz, 1H). Exchangeable NH proton not observed. LCMS (ESI) m/z: 444.2 [M+H]⁺.

### Example 175

### morpholino(8-(2-(pyridin-4-yl)pyrido[3,4-d]pyrimidin-4-yl)-2,8-diazaspiro[4.5]decan-3-yl)methanone (Compound 175)

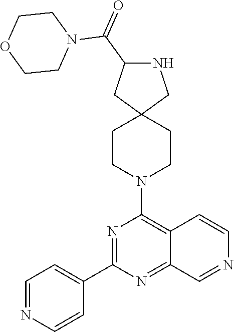 [SMILES: O=C(C1CC2(CCN(c3nc(-c4ccncc4)nc4cnccc34)CC2)CN1)N1CCOCC1]

Following the procedure described in Example 172, Step 2 and making non-critical variations as required to replace methylamine with morpholine the title compound was obtained (4.3 mg, 7.5% yield). ¹H NMR (400 MHz, DMSO-d₆) δ 9.26 (s, 1H), 8.79-8.73 (m, 2H), 8.59 (d, J=5.6 Hz, 1H), 8.36-8.31 (m, 2H), 7.90 (d, J=5.7 Hz, 1H), 4.04-3.82 (m, 5H), 3.57 (q, J=5.8 Hz, 5H), 3.53-3.44 (m, 4H), 2.96 (d, J=11.0 Hz, 1H), 2.65 (d, J=11.1 Hz, 1H), 2.04 (dd, J=12.9, 9.0 Hz, 1H), 1.81-1.73 (m, 2H), 1.73-1.67 (m, 1H), 1.64 (dd, J=12.8, 6.8 Hz, 1H). Exchangeable NH amine proton not observed. LCMS (ESI) m/z: 460.2 [M+H]⁺.

### Examples 176 and 177

### (3R,4S)-4-(8-(2-(pyridin-4-yl)pyrido[3,4-d]pyrimidin-4-yl)-2,8-diazaspiro[4.5]decan-2-yl)tetrahydrofuran-3-ol and (3S,4R)-4-(8-(2-(pyridin-4-yl)pyrido[3,4-d]pyrimidin-4-yl)-2,8-diazaspiro[4.5]decan-2-yl)tetrahydrofuran-3-ol (Compounds 176 and 177)

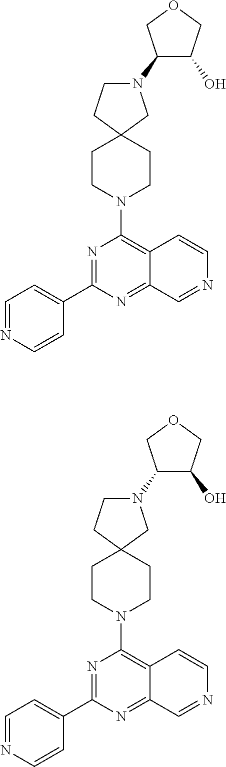 [SMILES: O[C@H]1COC[C@@H]1N1CCC2(CCN(c3nc(-c4ccncc4)nc4cnccc34)CC2)C1 ; O[C@@H]1COC[C@H]1N1CCC2(CCN(c3nc(-c4ccncc4)nc4cnccc34)CC2)C1]

Following the procedure described in Example 128 and making non-critical variations as required to replace 1,2-epoxycyclopentane with 3,6-dioxabicyclo[3.1.0]hexane, trans-4-(8-(2-(pyridin-4-yl)pyrido[3,4-d]pyrimidin-4-yl)-2,8-diazaspiro[4.5]decan-2-yl)tetrahydrofuran-3-ol was obtained as a mixture of enantiomers. The title compounds were obtained by separation on chiral SFC. The absolute stereochemistry of the products was assigned arbitrarily. Example 176: ¹H NMR (400 MHz, DMSO-d₆) δ 9.25 (s, 1H), 8.80-8.72 (m, 2H), 8.58 (d, J=5.6 Hz, 1H), 8.36-8.28 (m, 2H), 7.88 (d, J=5.6 Hz, 1H), 5.00 (d, J=4.4 Hz, 1H), 4.20-4.10 (m, 1H), 4.05-3.94 (m, 2H), 3.93-3.85 (m, 2H), 3.85-3.81 (m, 1H), 3.80-3.74 (m, 1H), 3.61-3.54 (m, 1H), 3.51-3.45 (m, 1H), 2.65-2.58 (m, 3H), 2.56-2.52 (m, 1H), 2.48-2.43 (m, 1H), 1.80-1.63 (m, 6H). LCMS (ESI) m/z: 433.1 [M+H]⁺. Example 177: ¹H NMR (400 MHz, DMSO-d₆) δ 9.26 (s, 1H), 8.83-8.72 (m, 2H), 8.59 (d, J=5.6 Hz, 1H), 8.37-8.28 (m, 2H), 7.89 (d, J=5.6 Hz, 1H), 5.00 (d, J=4.4 Hz, 1H), 4.19-4.12 (m, 1H), 4.07-3.94 (m, 2H), 3.93-3.85 (m, 2H), 3.85-3.81 (m, 1H), 3.80-3.75 (m, 1H), 3.60-3.54 (m, 1H), 3.51-3.43 (m, 1H), 2.66-2.58 (m, 3H), 2.56-2.52 (m, 1H), 2.48-2.43 (m, 1H), 1.80-1.64 (m, 6H). LCMS (ESI) m/z: 433.1 [M+H]⁺.

### Examples 178 and 179

### (1R,2R)-2-(8-(2-(pyridin-4-yl)pyrido[3,4-d]pyrimidin-4-yl)-2,8-diazaspiro[4.5]decan-2-yl)cyclobutan-1-ol and (1S,2S)-2-(8-(2-(pyridin-4-yl)pyrido[3,4-d]pyrimidin-4-yl)-2,8-diazaspiro[4.5]decan-2-yl)cyclobutan-1-ol (Compounds 178 and 179)

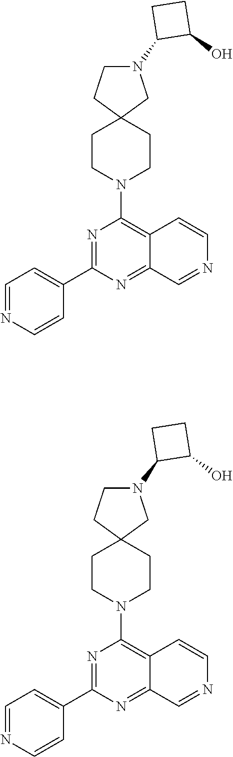 [SMILES: O[C@@H]1CC[C@H]1N1CCC2(CCN(c3nc(-c4ccncc4)nc4cnccc34)CC2)C1 ; O[C@H]1CC[C@@H]1N1CCC2(CCN(c3nc(-c4ccncc4)nc4cnccc34)CC2)C1]

### Step 1: 2-(8-(2-(pyridin-4-yl)pyrido[3,4-d]pyrimidin-4-yl)-2,8-diazaspiro[4.5]decan-2-yl)cyclobutanone

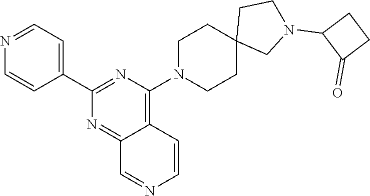 [SMILES: O=C1CCC1N1CCC2(CCN(c3nc(-c4ccncc4)nc4cnccc34)CC2)C1]

To a solution of 4-(2,8-diazaspiro[4.5]decan-8-yl)-2-(4-pyridyl)pyrido[3,4-d]pyrimidine hydrochloride (400 mg, 1 mmol) in MeOH (5 mL) was added N,N-diisopropylethylamine (0.2 mL, 0.12 mmol). The reaction mixture was stirred at room temperature for 5 minutes, and 1,2-bis((trimethylsilyl)oxy)cyclobut-1-ene (0.3 mL, 1.1 mmol) was added to this mixture. The mixture was stirred at room temperature for 5 hours. The reaction mixture was concentrated in vacuo. The crude residue was purified by silica gel chromatography (solvent gradient: 0-5% MeOH in DCM) to give the title compound (300 mg, 69%) as a yellow solid. LCMS (ESI) m/z: 415.0 [M+H]⁺.

### Step 2: trans-2-(8-(2-(pyridin-4-yl)pyrido[3,4-d]pyrimidin-4-yl)-2,8-diazaspiro[4.5]decan-2-yl)cyclobutanol and cis-2-(8-(2-(pyridin-4-yl)pyrido[3,4-d]pyrimidin-4-yl)-2,8-diazaspiro[4.5]decan-2-yl)cyclobutanol

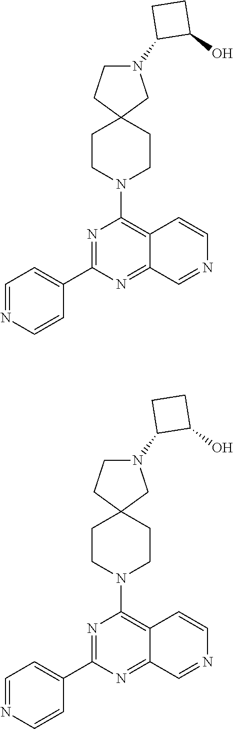 [SMILES: O[C@@H]1CC[C@H]1N1CCC2(CCN(c3nc(-c4ccncc4)nc4cnccc34)CC2)C1 ; O[C@H]1CC[C@H]1N1CCC2(CCN(c3nc(-c4ccncc4)nc4cnccc34)CC2)C1]

To a solution of 2-(8-(2-(pyridin-4-yl)pyrido[3,4-d]pyrimidin-4-yl)-2,8-diazaspiro[4.5]decan-2-yl)cyclobutanone (300 mg, 0.72 mmol) in MeOH (7 mL) was added sodium borohydride (80 mg, 2.2 mmol). The mixture was stirred at room temperature for 2 hours. The reaction was poured into sat. aq. NH₄Cl (20 mL), extracted with EtOAc (50 mL×2). The combined organic layers were washed with brine (30 mL), dried over anhydrous Na₂SO₄, filtered and concentrated in vacuo. The crude residue was purified by reverse phase chromatography (acetonitrile 28-58%/0.225% formic acid in water) to give the racemic trans isomer (35 mg, 12%) and the cis isomer (36 mg, 12%) both as a yellow solid. LCMS (ESI) m/z: 417.3 [M+H]⁺.

### Step 3: (1R,2R)-2-(8-(2-(pyridin-4-yl)pyrido[3,4-d]pyrimidin-4-yl)-2,8-diazaspiro[4.5]decan-2-yl)cyclobutan-1-ol and (1S,2S)-2-(8-(2-(pyridin-4-yl)pyrido[3,4-d]pyrimidin-4-yl)-2,8-diazaspiro[4.5]decan-2-yl)cyclobutan-1-ol

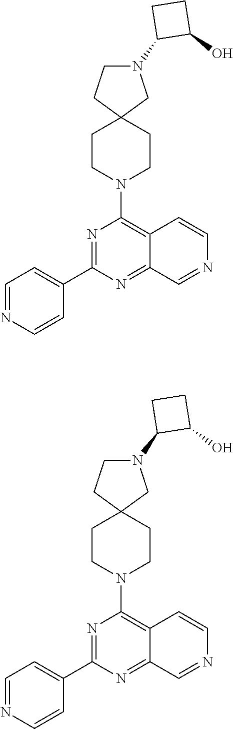 [SMILES: O[C@@H]1CC[C@H]1N1CCC2(CCN(c3nc(-c4ccncc4)nc4cnccc34)CC2)C1 ; O[C@H]1CC[C@@H]1N1CCC2(CCN(c3nc(-c4ccncc4)nc4cnccc34)CC2)C1]

trans-2-(8-(2-(pyridin-4-yl)pyrido[3,4-d]pyrimidin-4-yl)-2,8-diazaspiro[4.5]decan-2-yl)cyclobutan-1-ol (35 mg, 0.08 mmol) was separated by using chiral SFC (Chiralpak IG (250 mm*30 mm, 10 um), Supercritical CO₂/IPA+0.1% NH₄OH=40/60; 80 mL/min) to give the title compounds as white solids. The absolute configuration was arbitrarily assigned to each enantiomer. Example 178 (10 mg, first peak): ¹H NMR (400 MHz, DMSO-d₆) δ 9.26 (s, 1H), 8.77 (d, J=5.6 Hz, 2H), 8.59 (d, J=6.0 Hz, 1H), 8.32 (d, J=5.6 Hz, 2H), 7.89 (d, J=5.6 Hz, 1H), 5.17 (s, 1H), 4.03-3.81 (m, 5H), 2.61-2.53 (m, 3H), 2.47 (s, 2H), 2.43-2.36 (m, 1H), 1.99-1.90 (m, 1H), 1.81-1.66 (m, 6H), 1.50-1.36 (m, 1H), 1.26-1.21 (m, 1H). LCMS (ESI) m/z: 417.1 [M+H]⁺. Example 179 (5 mg, second peak): ¹H NMR (400 MHz, DMSO-d₆) δ 9.26 (s, 1H), 8.77 (d, J=5.6 Hz, 2H), 8.59 (d, J=5.6 Hz, 1H), 8.32 (d, J=5.6 Hz, 2H), 7.89 (d, J=6.0 Hz, 1H), 5.08 (d, J=7.2 Hz, 1H), 4.03-3.94 (m, 2H), 3.92-3.84 (m, 2H), 3.82-3.72 (m, 1H), 2.61-2.53 (m, 3H), 2.47 (s, 2H), 2.43-2.36 (m, 1H), 1.99-1.90 (m, 1H), 1.76-1.71 (m, 3H), 1.69-1.61 (m, 3H), 1.45-1.34 (m, 1H), 1.26-1.21 (m, 1H). LCMS (ESI) m/z: 417.1 [M+H]⁺.

### Examples 180 and 181

### (1S,2R)-2-(8-(2-(pyridin-4-yl)pyrido[3,4-d]pyrimidin-4-yl)-2,8-diazaspiro[4.5]decan-2-yl)cyclobutan-1-ol and (1R,2S)-2-(8-(2-(pyridin-4-yl)pyrido[3,4-d]pyrimidin-4-yl)-2,8-diazaspiro[4.5]decan-2-yl)cyclobutan-1-ol (Compounds 180 and 181)

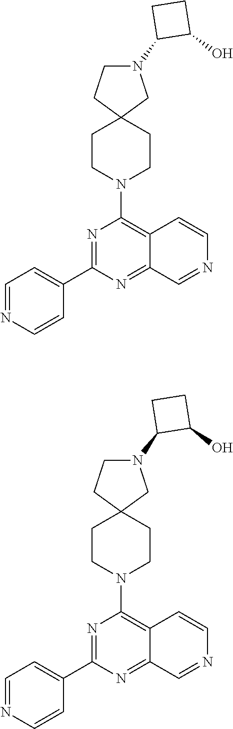 [SMILES: O[C@H]1CC[C@H]1N1CCC2(CCN(c3nc(-c4ccncc4)nc4cnccc34)CC2)C1 ; O[C@@H]1CC[C@@H]1N1CCC2(CCN(c3nc(-c4ccncc4)nc4cnccc34)CC2)C1]

cis-2-(8-(2-(pyridin-4-yl)pyrido[3,4-d]pyrimidin-4-yl)-2,8-diazaspiro[4.5]decan-2-yl)cyclobutan-1-ol (35 mg, 0.08 mmol) was separated by using chiral SFC (Chiralpak IG (250 mm*30 mm, 10 um), Supercritical CO₂/IPA+0.1% NH₄OH=40/60, 80 mL/min) to give the title compounds, both as white solids. The absolute configuration was arbitrarily assigned to each enantiomer. Example 180 (13 mg, first peak): ¹H NMR (400 MHz, DMSO-d₆) δ 9.25 (s, 1H), 8.76 (d, J=5.6 Hz, 2H), 8.58 (d, J=5.6 Hz, 1H), 8.32 (d, J=5.6 Hz, 2H), 7.88 (d, J=6.0 Hz, 1H), 4.79 (s, 1H), 4.08 (d, J=4.4 Hz, 1H), 4.01-3.84 (m, 4H), 2.89-2.86 (m, 1H), 2.78-2.69 (m, 1H), 2.66-2.56 (m, 2H), 2.09-1.97 (m, 1H), 1.87-1.66 (m, 9H), 1.26-1.21 (m, 1H). LCMS (ESI) m/z: 417.1 [M+H]⁺. Example 181 (12 mg, second peak): ¹H NMR (400 MHz, DMSO-d₆) δ 9.25 (s, 1H), 8.76 (d, J=5.6 Hz, 2H), 8.58 (d, J=5.6 Hz, 1H), 8.32 (d, J=5.6 Hz, 2H), 7.89 (d, J=5.6 Hz, 1H), 4.67 (s, 1H), 4.09-4.02 (m, 1H), 4.01-3.85 (m, 4H), 2.85-2.77 (m, 1H), 2.72-2.63 (m, 1H), 2.60-2.57 (m, 1H), 2.44-2.39 (m, 1H), 2.08-1.94 (m, 1H), 1.83-1.65 (m, 9H), 1.26-1.21 (m, 1H). LCMS (ESI) m/z: 417.1 [M+H]⁺.

### Examples 182

### 2-(3-fluoropyridin-4-yl)-4-(2,8-diazaspiro[4.5]decan-8-yl)pyrido[3,4-d]pyrimidine (Compound 182)

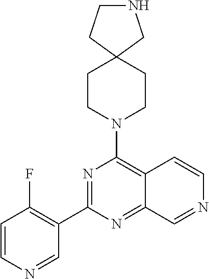 [SMILES: Fc1ccncc1-c1nc(N2CCC3(CCNC3)CC2)c2ccncc2n1]

Following the procedure described in Example 153, Step 2 and making non-critical variations as required to replace 3-chloro-4-(4,4,5,5-tetramethyl-1,3,2-dioxaborolan-2-yl)-1H-pyrazole with (3-fluoropyridin-4-yl)boronic acid the title compound was obtained. ¹H NMR (400 MHz, CD₃OD) δ 9.20 (s, 1H), 8.60 (d, J=3.2 Hz, 1H), 8.56 (d, J=6.0 Hz, 1H), 8.52 (d, J=4.8 Hz, 1H), 8.17-8.12 (m, 1H), 7.93 (d, J=5.6 Hz, 1H), 4.14-4.06 (m, 2H), 4.00-3.94 (m, 2H), 3.14 (t, J=7.2 Hz, 2H), 2.94 (s, 2H), 1.88 (t, J=7.2 Hz, 2H), 1.84-1.78 (m, 4H). LCMS (ESI) m/z: 365.3 [M+H]⁺.

### Example 183

### 4-(4-(2,8-diazaspiro[4.5]decan-8-yl)pyrido[3,4-d]pyrimidin-2-yl)nicotinonitrile (Compound 183)

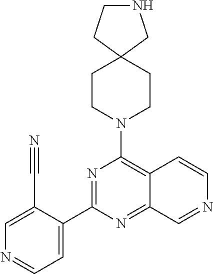 [SMILES: N#Cc1cnccc1-c1nc(N2CCC3(CCNC3)CC2)c2ccncc2n1]

Following the procedure described in Example 153, Step 2 and making non-critical variations as required to replace 3-chloro-4-(4,4,5,5-tetramethyl-1,3,2-dioxaborolan-2-yl)-1H-pyrazole with 4-(4,4,5,5-tetramethyl-1,3,2-dioxaborolan-2-yl)nicotinonitrile the title compound was obtained. ¹H NMR (400 MHz, CD₃OD) δ 9.30 (s, 1H), 9.07 (s, 1H), 8.94 (d, J=5.2 Hz, 1H), 8.63-8.57 (m, 2H), 8.50 (s, 1H), 7.98 (d, J=6.0 Hz, 1H), 4.30-4.20 (m, 2H), 4.15-4.05 (m, 2H), 3.45 (t, J=7.2 Hz, 2H), 3.24 (s, 2H), 2.10 (t, J=7.2 Hz, 2H), 1.96-1.86 (m, 4H). LCMS (ESI) m/z: 372.3 [M+H]⁺.

### Examples 184 and 185

### (1R,2R)-2-(8-(2-(5-methyl-1H-pyrazol-4-yl)pyrido[3,4-d]pyrimidin-4-yl)-2,8-diazaspiro[4.5]decan-2-yl)cyclopentan-1-ol and (1S,2S)-2-(8-(2-(5-methyl-1H-pyrazol-4-yl)pyrido[3,4-d]pyrimidin-4-yl)-2,8-diazaspiro[4.5]decan-2-yl)cyclopentan-1-ol (Compounds 184 and 185)

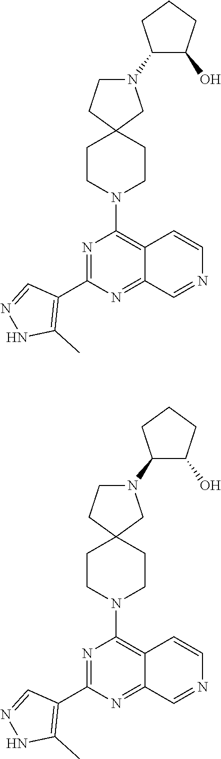 [SMILES: Cc1[nH]ncc1-c1nc(N2CCC3(CC2)CCN([C@@H]2CCC[C@H]2O)C3)c2ccncc2n1 ; Cc1[nH]ncc1-c1nc(N2CCC3(CC2)CCN([C@H]2CCC[C@@H]2O)C3)c2ccncc2n1]

Following the procedure described in Example 128 and making non-critical variations as required to replace 4-(2,8-diazaspiro[4.5]decan-8-yl)-2-(4-pyridyl)pyrido[3,4-d]pyrimidine with 2-(5-methyl-1H-pyrazol-4-yl)-4-(2,8-diazaspiro[4.5]decan-8-yl)pyrido[3,4-d]pyrimidine, trans-2-(8-(2-(5-methyl-1H-pyrazol-4-yl)pyrido[3,4-d]pyrimidin-4-yl)-2,8-diazaspiro[4.5]decan-2-yl)cyclopentan-1-ol was obtained as a mixture of enantiomers. The title compounds were obtained by separation on chiral SFC. The absolute stereochemistry of the products was assigned arbitrarily. Example 184: ¹H NMR (400 MHz, DMSO-d₆) δ 12.86 (s, 1H), 9.07 (s, 1H), 8.43 (d, J=5.6 Hz, 1H), 8.09 (s, 1H), 7.75 (d, J=5.6 Hz, 1H), 4.49 (d, J=4.8 Hz, 1H), 3.98-3.80 (m, 3H), 3.79-3.69 (m, 2H), 2.71-2.63 (m, 2H), 2.63-2.57 (m, 2H), 2.55-2.52 (m, 1H), 2.49-2.42 (m, 3H), 2.34-2.24 (m, 1H), 1.83-1.74 (m, 2H), 1.73-1.66 (m, 3H), 1.65-1.61 (m, 2H), 1.60-1.32 (m, 4H). LCMS (ESI) m/z: 434.2 [M+H]⁺. Example 185: ¹H NMR (400 MHz, DMSO-d₆) δ 12.85 (s, 1H), 9.07 (s, 1H), 8.43 (d, J=5.6 Hz, 1H), 8.08 (s, 1H), 7.75 (d, J=5.6 Hz, 1H), 4.48 (d, J=4.8 Hz, 1H), 3.96-3.79 (m, 3H), 3.78-3.68 (m, 2H), 2.71-2.63 (m, 2H), 2.62-2.57 (m, 2H), 2.55-2.52 (m, 1H), 2.48-2.40 (m, 3H), 2.35-2.24 (m, 1H), 1.83-1.74 (m, 2H), 1.73-1.67 (m, 3H), 1.65-1.62 (m, 2H), 1.61-1.28 (m, 4H). LCMS (ESI) m/z: 434.2 [M+H]⁺.

### Example 186

### 5-fluoro-2-(5-methyl-1H-pyrazol-4-yl)-4-(2,8-diazaspiro[4.5]decan-8-yl)pyrido[3,4-d]pyrimidine (Compound 186)

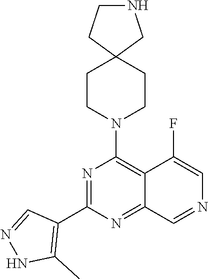 [SMILES: Cc1[nH]ncc1-c1nc(N2CCC3(CCNC3)CC2)c2c(F)cncc2n1]

Following the procedure described in Example 142, step 2 and making non-critical variations as required to replace 3-aminopyridine-4-carboxamide with 3-amino-5-fluoroisonicotinamide the title compound was obtained as an off-white solid. ¹H NMR (400 MHz, DMSO-d₆) δ 8.92 (s, 1H), 8.43-8.38 (m, 2H), 8.16 (s, 1H), 3.68-3.64 (m, 4H), 3.17-3.12 (m, 2H), 2.96 (s, 2H), 2.65 (s, 3H), 1.83-1.76 (m, 2H), 1.74-1.65 (m, 4H). LCMS (ESI) m/z: 368.0 [M+H].

### Example 187

### 8-(2-(pyridin-4-yl)pyrido[3,4-d]pyrimidin-4-yl)-2,8-diazaspiro[4.5]decan-4-ol (Compound 187)

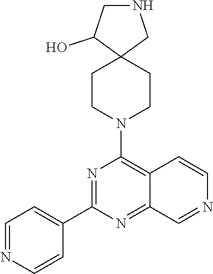 [SMILES: OC1CNCC12CCN(c1nc(-c3ccncc3)nc3cnccc13)CC2]

### Step 1: benzyl 4-hydroxy-1-oxo-2,8-diazaspiro[4.5]decane-8-carboxylate

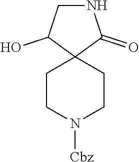 [SMILES: O=C(OCc1ccccc1)N1CCC2(CC1)C(=O)NCC2O]

To a solution of 1-benzyl 4-methyl piperidine-1,4-dicarboxylate (5.0 g, 18.0 mmol) in THF (50 mL) at −78° C. was added LiHMDS (39.7 mL, 39.7 mmol, 1M). The reaction mixture was warmed to room temperature and stirred for 1 hour and then cooled to −78° C., tert-butyl (2-oxoethyl)carbamate (3.4 g, 21.6 mmol) in THF (25 mL) was added to the reaction mixture at −78° C. And then the reaction was warmed to room temperature and stirred for 16 hours. The reaction was quenched with sat. aq. NH₄Cl (30 mL) and extracted with EtOAc (80 mL×2). The combined organic layers were dried over anhydrous Na₂SO₄, filtered and concentrated in vacuo. The crude residue was purified by silica gel chromatography (solvent gradient: 0-10% MeOH in DCM) to give the title compound (1.7 g, 31%) as a yellow solid. ¹H NMR (400 MHz, CDCl₃) δ 7.42-7.28 (m, 5H), 6.03 (s, 1H), 5.14 (s, 2H), 4.27-4.21 (m, 1H), 3.93-3.82 (m, 2H), 3.66-3.59 (m, 1H), 3.53-3.43 (m, 2H), 3.27-3.21 (m, 1H), 2.52 (s, 1H), 1.85-1.68 (m, 3H), 1.54-1.46 (m, 1H). LCMS (ESI) m/z: 305.1 [M+H].

### Step 2: benzyl 4-hydroxy-2,8-diazaspiro[4.5]decane-8-carboxylate

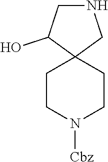 [SMILES: O=C(OCc1ccccc1)N1CCC2(CC1)CNCC2O]

To a solution of benzyl 4-hydroxy-1-oxo-2,8-diazaspiro[4.5]decane-8-carboxylate (300 mg, 0.99 mmol) in THF (9 mL) was added BH₃-THF complex (4.93 mL, 4.93 mmol, 1M) at 0° C. The mixture was heated to 70° C. and stirred for 16 hours. After cooling to room temperature, the reaction was quenched with MeOH (3 mL) and water (3 mL), and then the mixture was stirred for 30 minutes. The mixture was concentrated in vacuo, the residue was dissolved in MeOH (5 mL) and HCl (3 mL, 3 mmol, 1M) at room temperature. The mixture was heated to 70° C. and stirred for 16 hours. After cooling to room temperature, the mixture was concentrated in vacuo to give the title compound (300 mg, crude) as yellow oil that required no further purification. LCMS (ESI) m/z: 291.2 [M+H]⁺.

### Step 3: 8-benzyl 2-(tert-butyl) 4-hydroxy-2,8-diazaspiro[4.5]decane-2,8-dicarboxylate

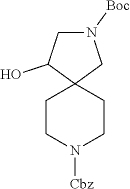 [SMILES: CC(C)(C)OC(=O)N1CC(O)C2(CCN(C(=O)OCc3ccccc3)CC2)C1]

To a solution of benzyl 4-hydroxy-2,8-diazaspiro[4.5]decane-8-carboxylate (300 mg, 1.03 mmol) in THF (5 mL) and water (2 mL) was added Na₂CO₃ (329 mg, 3.11 mmol) and di-tert-butyl dicarbonate (451 mg, 2.07 mmol). The mixture was stirred at room temperature for 16 h. The reaction mixture was diluted with water (30 mL), extracted with EtOAc (50 mL×2). The combined organic layers were dried over anhydrous Na₂SO₄, filtered and concentrated in vacuo. The crude residue was purified by column chromatography (0-60% EtOAc in petroleum ether) to give the title compound (120 mg, 30%) as colorless oil. ¹H NMR (400 MHz, CDCl₃) δ 7.40-7.29 (m, 5H), 5.14 (s, 2H), 4.07-3.71 (m, 3H), 3.67-3.57 (m, 1H), 3.45-3.01 (m, 5H), 1.82-1.64 (m, 2H), 1.47 (s, 9H), 1.45-1.35 (m, 2H). LCMS (ESI) m/z: 291.2 [M-100+H]⁺.

### Step 4: tert-butyl 4-hydroxy-2,8-diazaspiro[4.5]decane-2-carboxylate

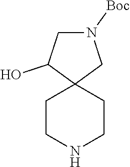 [SMILES: CC(C)(C)OC(=O)N1CC(O)C2(CCNCC2)C1]

To a solution of 8-benzyl 2-(tert-butyl) 4-hydroxy-2,8-diazaspiro[4.5]decane-2,8-dicarboxylate (110 mg, 0.28 mmol) in EtOAc (3 mL) was added 10% palladium on carbon (20 mg). The mixture was stirred at room temperature for 16 hours under hydrogen balloon (15 psi). The mixture was filtered and the filtrate was concentrated in vacuo to give the title compound (70 mg, crude) as yellow oil that required no further purification. LCMS (ESI) m/z: 257.2 [M+H]⁺.

### Step 5: 8-(2-(pyridin-4-yl)pyrido[3,4-d]pyrimidin-4-yl)-2,8-diazaspiro[4.5]decan-4-ol

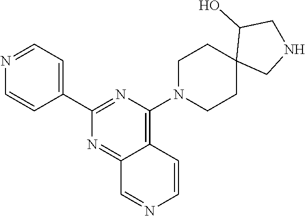 [SMILES: OC1CNCC12CCN(c1nc(-c3ccncc3)nc3cnccc13)CC2]

Following the procedure described in Example 101, Step 3 and making non-critical variations as required to replace tert-butyl 2,8-diazaspiro[4.5]decane-2-carboxylate with tert-butyl 4-hydroxy-2,8-diazaspiro[4.5]decane-2-carboxylate, the title compound was obtained as a white solid. ¹H NMR (400 MHz, DMSO-d₆) δ 9.26 (s, 1H), 8.77 (d, J=6.0 Hz, 2H), 8.59 (d, J=5.6 Hz, 1H), 8.33 (d, J=6.0 Hz, 2H), 7.91 (d, J=5.6 Hz, 1H), 4.27-4.17 (m, 2H), 3.94-3.89 (m, 1H), 3.82-3.64 (m, 2H), 3.56-3.48 (m, 2H), 3.31-3.29 (m, 2H), 3.20-3.14 (m, 1H), 2.01-1.92 (m, 1H), 1.78-1.65 (m, 2H), 1.61-1.53 (m, 1H). LCMS (ESI) m/z: 363.2 [M+H].

### Example 188

### 2-(5-fluoro-1H-pyrazol-4-yl)-4-(2,8-diazaspiro[4.5]decan-8-yl)pyrido[3,4-d]pyrimidine (Compound 188)

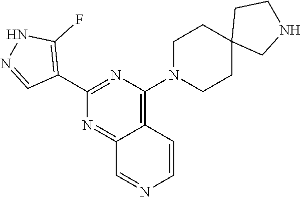 [SMILES: Fc1[nH]ncc1-c1nc(N2CCC3(CCNC3)CC2)c2ccncc2n1]

Following the procedure described in Example 153, Step 2 and making non-critical variations as required to replace 3-chloro-4-(4,4,5,5-tetramethyl-1,3,2-dioxaborolan-2-yl)-1H-pyrazole with 5-fluoro-4-(4,4,5,5-tetramethyl-1,3,2-dioxaborolan-2-yl)-1-((2-(trimethylsilyl)ethoxy)methyl)-1H-pyrazole the title compound was obtained. ¹H NMR (400 MHz, DMSO-d₆) δ 9.08 (s, 1H), 8.48 (d, J=5.6 Hz, 1H), 8.40 (s, 1H), 8.34 (s, 1H), 7.79 (d, J=5.6 Hz, 1H), 3.88-3.85 (m, 4H), 3.20-3.13 (m, 2H), 2.98 (s, 2H), 1.84-1.80 (m, 2H), 1.77-1.72 (m, 4H). LCMS (ESI) m/z: 353.9 [M+H].

### Example 189

### 6-((4-chloro-1H-pyrazol-1-yl)methyl)-4-(2-methyl-2,8-diazaspiro[4.5]decan-8-yl)-2-(pyridin-4-yl)pyrido[3,4-d]pyrimidine (Compound 189) formate

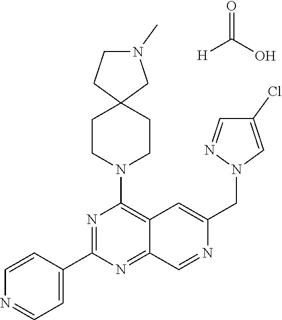

### Step 1: tert-butyl 8-(6-chloro-2-(pyridin-4-yl)pyrido[3,4-d]pyrimidin-4-yl)-2,8-diazaspiro[4.5]decane-2-carboxylate

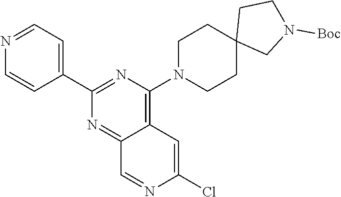 [SMILES: CC(C)(C)OC(=O)N1CCC2(CCN(c3nc(-c4ccncc4)nc4cnc(Cl)cc34)CC2)C1]

To a solution of 6-chloro-2-(4-pyridyl)pyrido[3,4-d]pyrimidin-4-ol (2 g, 7.7 mmol) and BOP (4.1 g, 9.3 mmol) in MeCN (20 mL) was added 1,8-diazabicyclo[5.4.0]undec-7-ene (1.7 g, 11.6 mmol). The reaction mixture was stirred at room temperature for 5 minutes, 2,8-diazaspiro[4.5]decane-2-carboxylate (2 g, 8.5 mmol) was added to this mixture. The mixture was stirred at room temperature for 16 hours and concentrated in vacuo. The crude residue was purified by silica gel chromatography (solvent gradient: 0-5% MeOH in DCM) to give the title compound (3.5 g, 7.3 mmol, 94%) as a yellow solid. LCMS (ESI) m/z: 481.5 [M+H]⁺

### Step 2: tert-butyl 8-(2-(pyridin-4-yl)-6-vinylpyrido[3,4-d]pyrimidin-4-yl)-2,8-diazaspiro[4.5]decane-2-carboxylate

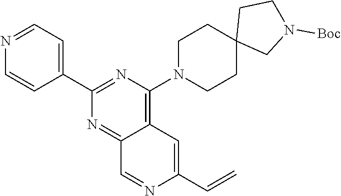 [SMILES: C=Cc1cc2c(N3CCC4(CCN(C(=O)OC(C)(C)C)C4)CC3)nc(-c3ccncc3)nc2cn1]

To a solution of potassium vinyltrifluoroborate (1.1 g, 8.3 mmol), tert-butyl 8-(6-chloro-2-(pyridin-4-yl)pyrido[3,4-d]pyrimidin-4-yl)-2,8-diazaspiro[4.5]decane-2-carboxylate (2 g, 4.2 mmol), triethylamine (1.7 mL, 12.5 mmol) in 1-propanol (15 mL) was added 1,1′-bis(diphenylphosphino)ferrocene palladium dichloride (300 mg, 0.4 mmol). The reaction mixture was heated to 100° C. under nitrogen atmosphere for 16 hours. After cooling to room temperature, the solvent was concentrated in vacuo. The crude residue was purified by silica gel chromatography (solvent gradient: 0-5% MeOH in DCM) to give the title compound (1.7 mg, 87%) as a yellow solid. LCMS (ESI) m/z: 473.6 [M+H]⁺

### Step 3: tert-butyl 8-(6-formyl-2-(pyridin-4-yl)pyrido[3,4-d]pyrimidin-4-yl)-2,8-diazaspiro[4.5]decane-2-carboxylate

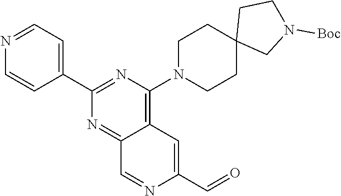 [SMILES: CC(C)(C)OC(=O)N1CCC2(CCN(c3nc(-c4ccncc4)nc4cnc(C=O)cc34)CC2)C1]

To a solution of tert-butyl 8-(2-(pyridin-4-yl)-6-vinylpyrido[3,4-d]pyrimidin-4-yl)-2,8-diazaspiro[4.5]decane-2-carboxylate (1.7 g, 3.6 mmol) in THF (6 mL) and water (6 mL) was added osmium tetroxide (180 mg, 0.72 mmol) at 0° C. The reaction mixture was stirred at 0° C. for 5 minutes, sodium periodate (1.5 g, 7.2 mmol) was added to this mixture. The reaction was allowed to warm to room temperature and stirred for 16 hours. The reaction was poured into sat. aq. NaHCO₃ (50 mL), extracted with EtOAc (60 mL×2). The combined organic layers were washed with brine (100 mL), dried over anhydrous Na₂SO₄, filtered and concentrated in vacuo. The residue was purified by silica gel chromatography (solvent gradient: 0-5% MeOH in DCM) to give the title compound (800 mg, 47%) as a brown solid. LCMS (ESI) m/z: 475.1 [M+H]⁺

### Step 4: tert-butyl 8-(6-(hydroxymethyl)-2-(pyridin-4-yl)pyrido[3,4-d]pyrimidin-4-yl)-2,8-diazaspiro[4.5]decane-2-carboxylate

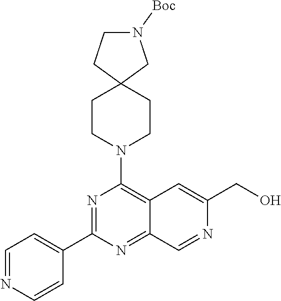 [SMILES: CC(C)(C)OC(=O)N1CCC2(CCN(c3nc(-c4ccncc4)nc4cnc(CO)cc34)CC2)C1]

To a solution of tert-butyl 8-(6-formyl-2-(pyridin-4-yl)pyrido[3,4-d]pyrimidin-4-yl)-2,8-diazaspiro[4.5]decane-2-carboxylate (800 mg, 1.7 mmol) in MeOH (7 mL) was added sodium borohydride (190 mg, 5 mmol). The mixture was stirred at room temperature for 2 hours. The reaction was poured into sat. aq. NH₄Cl (5 mL), extracted with EtOAc (20 mL×2). The combined organic layers were washed with brine (10 mL), dried over anhydrous Na₂SO₄, filtered and concentrated in vacuo. The residue was purified by silica gel chromatography (solvent gradient: 0-5% MeOH in DCM) to give the title compound (120 mg, 14.9%) as a yellow solid. LCMS (ESI) m/z: 477.1 [M+H]⁺

### Step 5: 6-(chloromethyl)-2-(pyridin-4-yl)-4-(2,8-diazaspiro[4.5]decan-8-yl)pyrido[3,4-d]pyrimidine

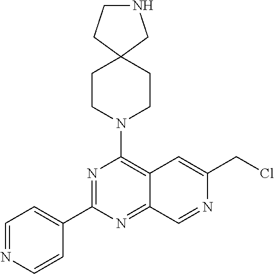 [SMILES: ClCc1cc2c(N3CCC4(CCNC4)CC3)nc(-c3ccncc3)nc2cn1]

To a solution of tert-butyl 8-(6-(hydroxymethyl)-2-(pyridin-4-yl)pyrido[3,4-d]pyrimidin-4-yl)-2,8-diazaspiro[4.5]decane-2-carboxylate (120 mg, 0.25 mmol) in DCM (4 mL) was added thionyl chloride (0.05 mL, 0.76 mmol) at 0° C. The reaction was allowed to warm to room temperature and stirred for 2 h. The mixture was concentrated in vacuo to give the title compound (90 mg, crude) as yellow oil that required no further purification. LCMS (ESI) m/z: 395.0 [M+H]⁺

### Step 6: tert-butyl 8-(6-(chloromethyl)-2-(pyridin-4-yl)pyrido[3,4-d]pyrimidin-4-yl)-2,8-diazaspiro[4.5]decane-2-carboxylate

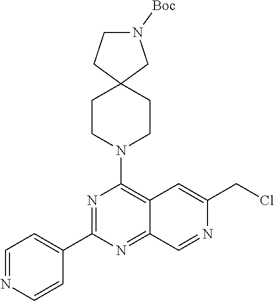 [SMILES: CC(C)(C)OC(=O)N1CCC2(CCN(c3nc(-c4ccncc4)nc4cnc(CCl)cc34)CC2)C1]

To a solution of 6-(chloromethyl)-2-(pyridin-4-yl)-4-(2,8-diazaspiro[4.5]decan-8-yl)pyrido[3,4-d]pyrimidine (90.0 mg, 0.23 mmol) in DCM (4 mL) was added triethylamine (0.04 mL, 0.27 mmol) and di-tert-butyldicarbonate (55 mg, 0.25 mmol). The solution was stirred at room temperature for 16 hours. The mixture was concentrated in vacuo to give the title compound (110 mg, crude) as yellow oil that required no further purification. LCMS (ESI) m/z: 495.1 [M+H]⁺

### Step 7: tert-butyl 8-(6-((4-chloro-1H-pyrazol-1-yl)methyl)-2-(pyridin-4-yl)pyrido[3,4-d]pyrimidin-4-yl)-2,8-diazaspiro[4.5]decane-2-carboxylate

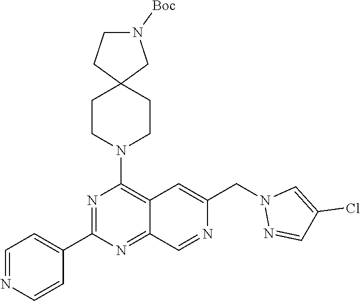 [SMILES: CC(C)(C)OC(=O)N1CCC2(CCN(c3nc(-c4ccncc4)nc4cnc(Cn5cc(Cl)cn5)cc34)CC2)C1]

To a solution of tert-butyl 8-(6-(chloromethyl)-2-(pyridin-4-yl)pyrido[3,4-d]pyrimidin-4-yl)-2,8-diazaspiro[4.5]decane-2-carboxylate (110 mg, 0.22 mmol), 4-chloropyrazole (0.03 mL, 0.33 mmol), tetrabutylammonium hydroxide (6 drops) in toluene (4 mL) was added 40% aqueous sodium hydroxide (1 mL). The reaction mixture was heated to 100° C. under nitrogen atmosphere for 16 hours. After cooling to room temperature, the mixture was diluted with EtOAc (50 mL), washed with water (15 mL×2) and brine (30 mL). The organic layer was dried over anhydrous Na₂SO₄, filtered and concentrated in vacuo. The crude residue was purified by silica gel chromatography (solvent gradient: 0-5% MeOH in DCM) to give the title compound (50 mg, 40%) as a yellow solid. LCMS (ESI) m/z: 561.1 [M+H]⁺

### Step 8: 6-((4-chloro-1H-pyrazol-1-yl)methyl)-2-(pyridin-4-yl)-4-(2,8-diazaspiro[4.5]decan-8-yl)pyrido[3,4-d]pyrimidine hydrochloride

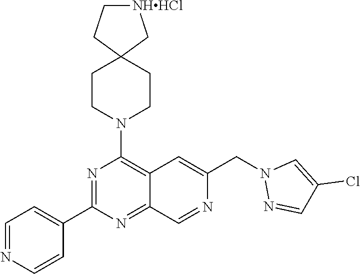

To a solution of tert-butyl 8-(6-((4-chloro-1H-pyrazol-1-yl)methyl)-2-(pyridin-4-yl)pyrido[3,4-d]pyrimidin-4-yl)-2,8-diazaspiro[4.5]decane-2-carboxylate (50 mg, 0.09 mmol) in EtOAc (2 mL) was added 4M HCl in EtOAc (5 mL, 20.0 mmol). The mixture was stirred at room temperature for 2 hours. The mixture was concentrated in vacuo to give the title compound (40 mg, crude) as a yellow solid that required no further purification. LCMS (ESI) m/z: 461.1 [M+H]⁺.

### Step 9: 6-((4-chloro-1H-pyrazol-1-yl)methyl)-4-(2-methyl-2,8-diazaspiro[4.5]decan-8-yl)-2-(pyridin-4-yl)pyrido[3,4-d]pyrimidine formate

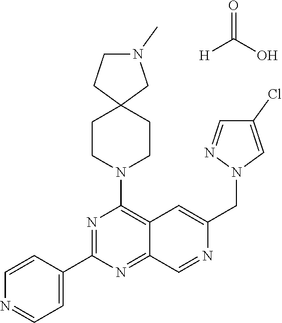

Following the procedure described in Example 102 and making non-critical variations as required to replace 4-(2,8-diazaspiro[4.5]decan-8-yl)-2-(4-pyridyl)pyrido[3,4-d]pyrimidine hydrochloride with 6-((4-chloro-1H-pyrazol-1-yl)methyl)-2-(pyridin-4-yl)-4-(2,8-diazaspiro[4.5]decan-8-yl)pyrido[3,4-d]pyrimidine hydrochloride, the title compound was obtained as a yellow solid. ¹H NMR (400 MHz, DMSO-d₆) δ 9.22 (s, 1H), 8.79-8.73 (m, 2H), 8.32-8.28 (m, 2H), 8.25 (s, 1H), 8.19 (s, 1H), 7.65 (s, 1H), 7.42 (s, 1H), 5.60 (s, 2H), 4.12-3.63 (m, 4H), 2.78-2.67 (m, 2H), 2.60 (s, 2H), 2.38 (s, 3H), 1.79-1.69 (m, 6H). LCMS (ESI) m/z: 475.1 [M+H]⁺.

### Example 190

### 6-((4-chloro-1H-pyrazol-1-yl)methyl)-2-(3-methyl-1H-pyrazol-4-yl)-4-(2-methyl-2,8-diazaspiro[4.5]decan-8-yl)pyrido[3,4-d]pyrimidine (Compound 190) formate

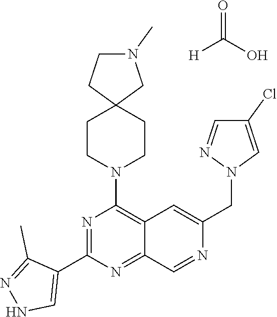

### Step 1: tert-butyl 8-(6-formyl-2-(3-methyl-1-((2-(trimethylsilyl)ethoxy)methyl)-1H-pyrazol-4-yl)pyrido[3,4-d]pyrimidin-4-yl)-2,8-diazaspiro[4.5]decane-2-carboxylate

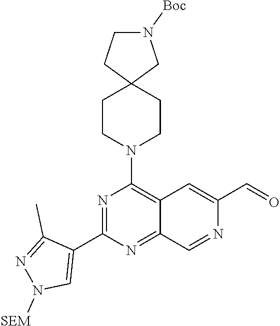 [SMILES: Cc1nn(COCC[Si](C)(C)C)cc1-c1nc(N2CCC3(CCN(C(=O)OC(C)(C)C)C3)CC2)c2cc(C=O)ncc2n1]

Following the procedure described in Example 189, Step 1 and making non-critical variations as required to replace 6-chloro-2-(pyridine-4-yl)pyrido[3,4-d]pyrimidin-4-ol with 6-chloro-2-(3-methyl-1-((2-(trimethylsilyl)ethoxy)methyl)-1H-pyrazol-4-yl)pyrido[3,4-d]pyrimidin-4-ol, the title compound was obtained as a yellow solid. LCMS (ESI) m/z: 608.6 [M+H]⁺.

### Step 2: 6-((4-chloro-1H-pyrazol-1-yl)methyl)-2-(3-methyl-1H-pyrazol-4-yl)-4-(2-methyl-2,8-diazaspiro[4.5]decan-8-yl)pyrido[3,4-d]pyrimidine formate

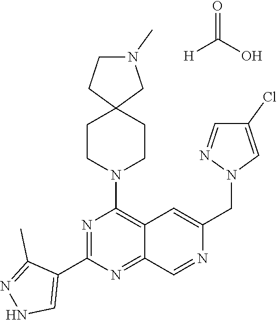

Following the procedure described in Example 189, Steps 4-9 and making non-critical variations as required to replace tert-butyl 8-(6-formyl-2-(pyridin-4-yl)pyrido[3,4-d]pyrimidin-4-yl)-2,8-diazaspiro[4.5]decane-2-carboxylate with tert-butyl 8-(6-formyl-2-(3-methyl-1-((2-(trimethylsilyl)ethoxy)methyl)-1H-pyrazol-4-yl)pyrido[3,4-d]pyrimidin-4-yl)-2,8-diazaspiro[4.5]decane-2-carboxylate, the title compound was obtained as a yellow solid. ¹H NMR (400 MHz, DMSO-d₆) δ 9.02 (s, 1H), 8.30 (s, 1H), 8.16 (s, 1H), 8.13 (s, 1H), 7.62 (s, 1H), 7.31 (s, 1H), 5.54 (s, 2H), 3.75-3.62 (im, 4H), 2.88-2.84 (m, 2H), 2.73 (s, 2H), 2.63 (s, 3H), 2.47 (s, 3H), 1.81-1.71 (m, 2H), 1.76-1.64 (m, 4H). LCMS (ESI) m/z: 478.5 [M+H]⁺.

### Example 191

### 5-cyclopropyl-2-(pyridin-4-yl)-4-(2,8-diazaspiro[4.5]decan-8-yl)pyrido[3,4-d]pyrimidine (Compound 191)

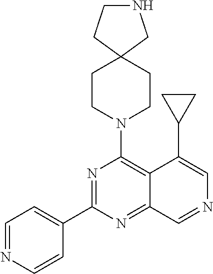 [SMILES: c1cc(-c2nc(N3CCC4(CCNC4)CC3)c3c(C4CC4)cncc3n2)ccn1]

Following the procedure described in Example 162, Step 3 and making non-critical variations as required to replace methylboronic acid with cyclopropylboronic acid the title compound was obtained. ¹H NMR (400 MHz, DMSO) δ 8.99 (s, 1H), 8.77 (d, J=6.0 Hz, 2H), 8.39 (s, 1H), 8.32 (d, J=6.0 Hz, 2H), 8.10 (s, 1H), 3.88-3.69 (m, 4H), 3.13-3.03 (m, 2H), 3.00-2.88 (m, 1H), 2.87-2.71 (m, 1H), 2.63-2.53 (m, 1H), 1.89-1.57 (m, 6H), 1.30-1.22 (m, 2H), 1.08-1.00 (m, 2H). LCMS (ESI) m/z: 387.3 [M+H]⁺.

### Example 192

### 2-(pyridin-4-yl)-4-(2,8-diazaspiro[4.5]decan-8-yl)-8-(2,2,2-trifluoroethyl)pyrido[3,4-d]pyrimidine (Compound 192)

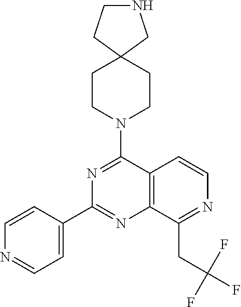 [SMILES: FC(F)(F)Cc1nccc2c(N3CCC4(CCNC4)CC3)nc(-c3ccncc3)nc12]

### Step 1: 2-(Pyridin-4-yl)-8-(2,2,2-trifluoroethyl)pyrido[3,4-d]pyrimidin-4-ol and 2-(pyridin-4-yl)-6-(2,2,2-trifluoroethyl)pyrido[3,4-d]pyrimidin-4-ol

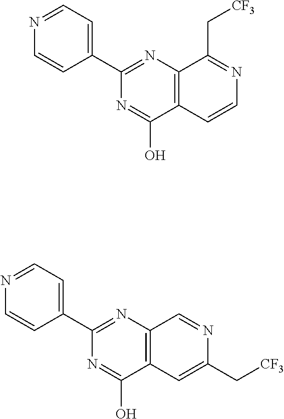 [SMILES: Oc1nc(-c2ccncc2)nc2c(CC(F)(F)F)nccc12 ; Oc1nc(-c2ccncc2)nc2cnc(CC(F)(F)F)cc12]

To a stirred solution of 4-chloro-2-(4-pyridyl)pyrido[3,4-d]pyrimidine (600 mg, 2.47 mmol) and zinc trifluoroethanesulfinate (1576 mg, 7.42 mmol) in DMSO (8.2 mL) and H₂O (0.82 mL) at 0° C. was added dropwise tert-butyl hydroperoxide in H₂O (2.0 mL, 14.8 mmol) over 15 minutes. The reaction mixture was warmed to room temperature for 15 minutes, and then heated to 50° C. for 3 hours and diluted with isopropyl acetate. The organic layer was washed with 50% brine and brine, dried over Na₂SO₄, filtered, and evaporated under reduced pressure. The crude product was purified by column chromatography (MeOH/iPrOAc) to provide 2-(pyridin-4-yl)-8-(2,2,2-trifluoroethyl)pyrido[3,4-d]pyrimidin-4-ol as an oil (110.2 mg, 14.6% yield). LCMS (ESI) m/z: 307 [M+H]⁺. ¹H NMR (400 MHz, DMSO) δ 13.17 (br s, 1H), 8.86-8.83 (m, 2H), 8.71 (d, J=5.1 Hz, 1H), 8.19-8.16 (m, 2H), 8.04 (d, J=5.1 Hz, 1H), 4.38 (q, J=11.2 Hz, 2H). Fractions containing a 1:1 mixture of 2-(pyridin-4-yl)-8-(2,2,2-trifluoroethyl)pyrido[3,4-d]pyrimidin-4-ol and 2-(pyridin-4-yl)-6-(2,2,2-trifluoroethyl)pyrido[3,4-d]pyrimidin-4-ol were also isolated as an oil (85.6 mg, 11.3% yield). LCMS (ESI) m/z: 307 [M+H]⁺.

### Step 2: tert-Butyl 8-(2-(pyridin-4-yl)-8-(2,2,2-trifluoroethyl)pyrido[3,4-d]pyrimidin-4-yl)-2,8-diazaspiro[4.5]decane-2-carboxylate

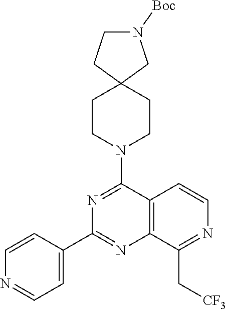 [SMILES: CC(C)(C)OC(=O)N1CCC2(CCN(c3nc(-c4ccncc4)nc4c(CC(F)(F)F)nccc34)CC2)C1]

A solution of 2-(pyridin-4-yl)-8-(2,2,2-trifluoroethyl)pyrido[3,4-d]pyrimidin-4-ol (105 mg, 0.34 mmol), 2,4,6-triisopropylbenzenesulfonyl chloride (128.5 mg, 0.41 mmol), DMAP (4.3 mg, 0.034 mmol), and DIPEA (0.18 mL, 1.03 mmol) in DMA (1.2 mL) was stirred at room temperature for 30 minutes. To this was added tert-butyl 2,8-diazaspiro[4.5]decane-2-carboxylate (99 mg, 0.41 mmol), and the reaction mixture was stirred at room temperature for 2 days and then diluted with isopropyl acetate. The organic layer was washed with saturated NaHCO₃, water and brine, dried over Na₂SO₄, filtered, and evaporated under reduced pressure. The crude was purified by column chromatography (MeOH/iPrOAc) to provide tert-butyl 8-(2-(pyridin-4-yl)-8-(2,2,2-trifluoroethyl)pyrido[3,4-d]pyrimidin-4-yl)-2,8-diazaspiro[4.5]decane-2-carboxylate as an oil (165.5 mg, 91.3% yield). LCMS (ESI) m/z: 529.2 [M+H]⁺. ¹H NMR (400 MHz, DMSO) δ 8.83-8.76 (m, 2H), 8.59 (d, J=5.8 Hz, 1H), 8.40-8.34 (m, 2H), 7.91 (d, J=5.7 Hz, 1H), 4.49 (q, J=11.3 Hz, 2H), 4.09-3.89 (m, 4H), 3.39-3.33 (m, 2H), 3.25-3.19 (m, 2H), 1.92-1.68 (m, 6H), 1.41 (s, 9H).

### Step 3: 2-(Pyridin-4-yl)-4-(2,8-diazaspiro[4.5]decan-8-yl)-8-(2,2,2-trifluoroethyl)pyrido[3,4-d]pyrimidine

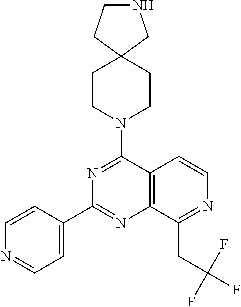 [SMILES: FC(F)(F)Cc1nccc2c(N3CCC4(CCNC4)CC3)nc(-c3ccncc3)nc12]

A mixture of tert-butyl 8-(2-(pyridin-4-yl)-8-(2,2,2-trifluoroethyl)pyrido[3,4-d]pyrimidin-4-yl)-2,8-diazaspiro[4.5]decane-2-carboxylate (165.5 mg, 0.31 mmol) in TFA (3.1 mL) and DCM (3.1 mL) was stirred at room temperature for 3 hours. Volatile solvents were removed under reduced pressure. The crude residue was basified with saturated NaHCO₃ and extracted with isopropyl acetate (3X). The combined organic layers were washed with water and brine, dried over Na₂SO₄, filtered, and evaporated under reduced pressure. The crude compound was purified by HPLC to give 2-(pyridin-4-yl)-4-(2,8-diazaspiro[4.5]decan-8-yl)-8-(2,2,2-trifluoroethyl)pyrido[3,4-d]pyrimidine as a white solid (27.5 mg, 20.5% yield). LCMS (ESI) m/z: 429.1 [M+H]⁺. ¹H NMR (400 MHz, DMSO) δ 8.82-8.78 (m, 2H), 8.59 (d, J=5.6 Hz, 1H), 8.39-8.35 (m, 2H), 7.91 (d, J=5.7 Hz, 1H), 4.49 (q, J=11.3 Hz, 2H), 4.09-3.87 (m, 4H), 3.07-2.99 (m, 2H), 2.85 (s, 2H), 1.81-1.68 (m, 6H); NH hidden.

### Example 193

### 2-(pyridin-4-yl)-4-(2,8-diazaspiro[4.5]decan-8-yl)-6-(2,2,2-trifluoroethyl)pyrido[3,4-d]pyrimidine (Compound 193)

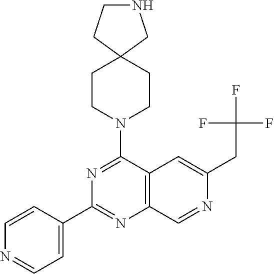 [SMILES: FC(F)(F)Cc1cc2c(N3CCC4(CCNC4)CC3)nc(-c3ccncc3)nc2cn1]

### Step 1: tert-Butyl 8-(2-(pyridin-4-yl)-6-(2,2,2-trifluoroethyl)pyrido[3,4-d]pyrimidin-4-yl)-2,8-diazaspiro[4.5]decane-2-carboxylate and tert-butyl 8-(2-(pyridin-4-yl)-8-(2,2,2-trifluoroethyl)pyrido[3,4-d]pyrimidin-4-yl)-2,8-diazaspiro[4.5]decane-2-carboxylate

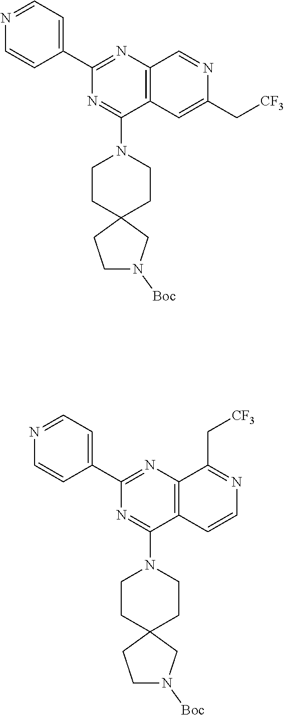 [SMILES: CC(C)(C)OC(=O)N1CCC2(CCN(c3nc(-c4ccncc4)nc4cnc(CC(F)(F)F)cc34)CC2)C1 ; CC(C)(C)OC(=O)N1CCC2(CCN(c3nc(-c4ccncc4)nc4c(CC(F)(F)F)nccc34)CC2)C1]

A 1:1 mixture of 2-(pyridin-4-yl)-6-(2,2,2-trifluoroethyl)pyrido[3,4-d]pyrimidin-4-ol and 2-(pyridin-4-yl)-8-(2,2,2-trifluoroethyl)pyrido[3,4-d]pyrimidin-4-ol (85 mg, 0.28 mmol), 2,4,6-triisopropylbenzenesulfonyl chloride (104 mg, 0.33 mmol), DMAP (3.5 mg, 0.03 mmol), and DIPEA (0.15 mL, 0.83 mmol) in DMA (1.0 mL) was stirred at room temperature for 30 minutes. To this was added tert-butyl 2,8-diazaspiro[4.5]decane-2-carboxylate (80 mg, 0.33 mmol), and the reaction mixture was stirred at room temperature for 2 days and diluted with isopropyl acetate. The organic layer was washed with saturated NaHCO₃, water and brine, dried over Na₂SO₄, filtered, and evaporated under reduced pressure. The crude was purified by column chromatography (MeOH/iPrOAc), and the compound mixture was separated by using achiral SFC (Chiralcel OX (150.0 mm*21.2 mm, 5 m), Supercritical CO₂/MeOH+0.1% NH₄OH=30/100 isocratic; 70 mL/min) to give tert-butyl 8-(2-(pyridin-4-yl)-6-(2,2,2-trifluoroethyl)pyrido[3,4-d]pyrimidin-4-yl)-2,8-diazaspiro[4.5]decane-2-carboxylate (first peak: 6.4 mg, 4.4% yield) as a white solid. LCMS (ESI) m/z: 529.2 [M+H]⁺. ¹H NMR (400 MHz, DMSO) δ 9.26 (s, 1H), 8.81-8.75 (m, 2H), 8.36-8.31 (m, 2H), 8.00 (s, 1H), 4.11-3.89 (m, 6H), 3.40-3.31 (m, 2H), 3.26-3.19 (m, 2H), 1.91-1.68 (m, 6H), 1.41 (s, 9H). The second eluant provided tert-butyl 8-(2-(pyridin-4-yl)-8-(2,2,2-trifluoroethyl)pyrido[3,4-d]pyrimidin-4-yl)-2,8-diazaspiro[4.5]decane-2-carboxylate (8.6 mg, 5.9% yield) as a white solid. LCMS (ESI) m/z: 529.2 [M+H]. ¹H NMR (400 MHz, DMSO) δ 8.83-8.76 (m, 2H), 8.59 (d, J=5.8 Hz, 1H), 8.40-8.34 (m, 2H), 7.91 (d, J=5.7 Hz, 1H), 4.49 (q, J=11.3 Hz, 2H), 4.09-3.89 (m, 4H), 3.39-3.33 (m, 2H), 3.25-3.19 (m, 2H), 1.92-1.68 (m, 6H), 1.41 (s, 9H).

### Step 2: 2-(Pyridin-4-yl)-4-(2,8-diazaspiro[4.5]decan-8-yl)-6-(2,2,2-trifluoroethyl)pyrido[3,4-d]pyrimidine

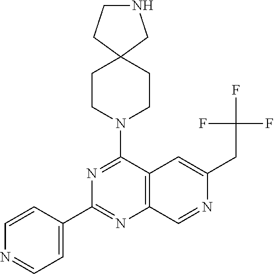 [SMILES: FC(F)(F)Cc1cc2c(N3CCC4(CCNC4)CC3)nc(-c3ccncc3)nc2cn1]

Following the procedure described in Example 192, Step 3, and making non-critical variation as required to replace tert-butyl 8-(2-(pyridin-4-yl)-8-(2,2,2-trifluoroethyl)pyrido[3,4-d]pyrimidin-4-yl)-2,8-diazaspiro[4.5]decane-2-carboxylate with tert-butyl 8-(2-(pyridin-4-yl)-6-(2,2,2-trifluoroethyl)pyrido[3,4-d]pyrimidin-4-yl)-2,8-diazaspiro[4.5]decane-2-carboxylate, the title compound was obtained as a solid (3.7 mg, 73% yield). LCMS (ESI) m/z: 429.1 [M+H]⁺. ¹H NMR (400 MHz, DMSO) δ 9.27 (s, 1H), 8.82-8.75 (m, 2H), 8.35-8.32 (m, 2H), 7.99 (s, 1H), 4.12-3.88 (m, 6H), 3.19-3.08 (m, 2H), 2.95 (s, 2H), 1.86-1.73 (m, 6H); NH hidden.

### Example 194

### 2-(3-methyl-1H-pyrazol-4-yl)-4-(2-methyl-2,8-diazaspiro[4.5]decan-8-yl)-6-(2,2,2-trifluoroethyl)pyrido[3,4-d]pyrimidine (Compound 194) formate

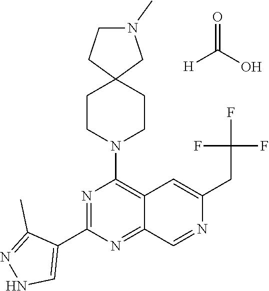 [SMILES: Cc1n[nH]cc1-c1nc(N2CCC3(CCN(C)C3)CC2)c2cc(CC(F)(F)F)ncc2n1 ; O=CO]

### Step 1: tert-butyl 8-(2-(3-methyl-1-((2-(trimethylsilyl)ethoxy)methyl)-1H-pyrazol-4-yl)-6-(2,2,2-trifluoro-1l-hydroxyethyl)pyrido[3,4-d]pyrimidin-4-yl)-2,8-diazaspiro[4.5]decane-2-carboxylate

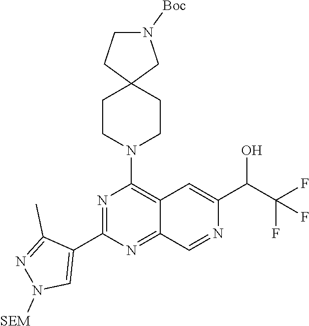 [SMILES: Cc1nn(COCC[Si](C)(C)C)cc1-c1nc(N2CCC3(CCN(C(=O)OC(C)(C)C)C3)CC2)c2cc(C(O)C(F)(F)F)ncc2n1]

To a solution of tert-butyl 8-(6-formyl-2-(3-methyl-1-((2-(trimethylsilyl)ethoxy)methyl)-1H-pyrazol-4-yl)pyrido[3,4-d]pyrimidin-4-yl)-2,8-diazaspiro[4.5]decane-2-carboxylate (400 mg, 0.66 mmol) in THF (3 mL) was added (trifluoromethyl)trimethylsilane (561 mg, 3.95 mmol) and tetrabutylammonium fluoride (3.95 mL, 3.95 mmol, 1M). The mixture was stirred at room temperature for 16 hours under nitrogen atmosphere. The reaction was quenched with water (20 mL) and extracted with EtOAc (50 mL). The organic layer was dried over anhydrous Na₂SO₄, filtered and concentrated in vacuo. The crude residue was purified by silica gel chromatography (solvent gradient: 0-5% MeOH in DCM) to give the title compound (300 mg, 67%) as a yellow solid. LCMS (ESI) m/z: 678.8 [M+H]⁺.

### Step 2: tert-butyl 8-(2-(3-methyl-1-((2-(trimethylsilyl)ethoxy)methyl)-1H-pyrazol-4-yl)-6-(2,2,2-trifluoro-1-((methylsulfonyl)oxy)ethyl)pyrido[3,4-d]pyrimidin-4-yl)-2,8-diazaspiro[4.5]decane-2-carboxylate

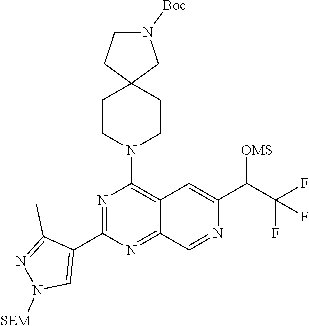

To s solution of tert-butyl 8-(2-(3-methyl-1-((2-(trimethylsilyl)ethoxy)methyl)-1H-pyrazol-4-yl)-6-(2,2,2-trifluoro-1-hydroxyethyl)pyrido[3,4-d]pyrimidin-4-yl)-2,8-diazaspiro[4.5]decane-2-carboxylate (100 mg, 0.15 mmol) in DCM (3 mL) was added N,N-diisopropylethylamine (0.08 mL, 0.44 mmol) and methanesulfonyl chloride (0.03 mL, 0.44 mmol). The mixture was stirred at room temperature for 3 hours under nitrogen atmosphere. The reaction was quenched with sat. aq. NaHCO₃ (20 mL) and extracted with DCM (30 mL×2). The combined organic layers were dried over anhydrous Na₂SO₄, filtered and concentrated in vacuo. The crude residue was purified by silica gel chromatography (solvent gradient: 0-5% MeOH in DCM) to give the title compound (50 mg, 44%) as a yellow solid. LCMS (ESI) m/z: 756.3 [M+H]⁺.

### Step 3: tert-butyl 8-(2-(3-methyl-1-((2-(trimethylsilyl)ethoxy)methyl)-1H-pyrazol-4-yl)-6-(2,2,2-trifluoroethyl)pyrido[3,4-d]pyrimidin-4-yl)-2,8-diazaspiro[4.5]decane-2-carboxylate

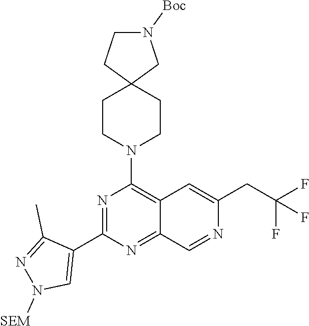 [SMILES: Cc1nn(COCC[Si](C)(C)C)cc1-c1nc(N2CCC3(CCN(C(=O)OC(C)(C)C)C3)CC2)c2cc(CC(F)(F)F)ncc2n1]

To a solution of tert-butyl 8-(2-(3-methyl-1-((2-(trimethylsilyl)ethoxy)methyl)-1H-pyrazol-4-yl)-6-(2,2,2-trifluoro-1-((methylsulfonyl)oxy)ethyl)pyrido[3,4-d]pyrimidin-4-yl)-2,8-diazaspiro[4.5]decane-2-carboxylate (50 mg, 0.07 mmol) in MeOH (1 mL) was added 10% palladium on carbon (10 mg). The mixture was stirred at room temperature for 2 hours under hydrogen balloon (15 psi). The mixture was filtered and the filtrate was concentrated in vacuo to give the title compound (30 mg, crude) as a yellow solid that required no further purification. LCMS (ESI) m/z: 662.4 [M+H]⁺.

### Step 4: 2-(3-methyl-1H-pyrazol-4-yl)-4-(2,8-diazaspiro[4.5]decan-8-yl)-6-(2,2,2-trifluoroethyl)pyrido[3,4-d]pyrimidine trifluoroacetate

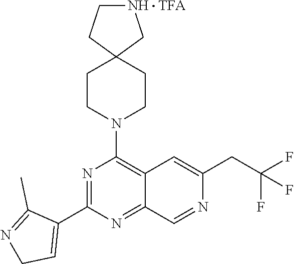

To a solution of tert-butyl 8-(2-(3-methyl-1-((2-(trimethylsilyl)ethoxy)methyl)-1H-pyrazol-4-yl)-6-(2,2,2-trifluoroethyl)pyrido[3,4-d]pyrimidin-4-yl)-2,8-diazaspiro[4.5]decane-2-carboxylate (25 mg, 0.04 mmol) in DCM (1 mL) was added trifluoroacetic acid (0.03 mL, 0.38 mmol). The mixture was stirred at room temperature for 2 hours. The mixture was concentrated in vacuo to give the title compound (15 mg, crude) as a yellow solid that required no further purification. LCMS (ESI) m/z: 432.2 [M+H].

### Step 5: 2-(3-methyl-1H-pyrazol-4-yl)-4-(2-methyl-2,8-diazaspiro[4.5]decan-8-yl)-6-(2,2,2-trifluoroethyl)pyrido[3,4-d]pyrimidine formate

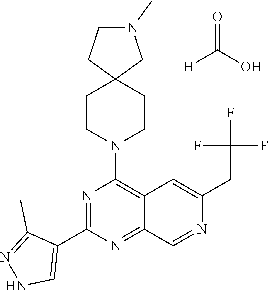 [SMILES: Cc1n[nH]cc1-c1nc(N2CCC3(CCN(C)C3)CC2)c2cc(CC(F)(F)F)ncc2n1 ; O=CO]

Following the procedure described in Example 102 and making non-critical variations as required to replace 4-(2,8-diazaspiro[4.5]decan-8-yl)-2-(4-pyridyl)pyrido[3,4-d]pyrimidine hydrochloride with 2-(3-methyl-1H-pyrazol-4-yl)-4-(2,8-diazaspiro[4.5]decan-8-yl)-6-(2,2,2-trifluoroethyl)pyrido[3,4-d]pyrimidine trifluoroacetate, the title compound was obtained as a yellow solid. ¹H NMR (400 MHz, DMSO-d₆) δ 9.06 (s, 1H), 8.22 (s, 1H), 8.14 (s, 1H), 7.85 (s, 1H), 4.02-3.92 (m, 2H), 3.86-3.80 (m, 2H), 3.79-3.74 (m, 2H), 2.65 (s, 3H), 2.59 (t, J=6.8 Hz, 2H), 2.47 (s, 2H), 2.29 (s, 3H), 1.78-1.66 (m, 6H). LCMS (ESI) m/z: 446.1 [M+H]⁺.

### Example 195

### 4-(2-methyl-2,8-diazaspiro[4.5]decan-8-yl)-2-(pyridin-4-yl)-6-(2,2,2-trifluoroethyl)pyrido[3,4-d]pyrimidine (Compound 195)

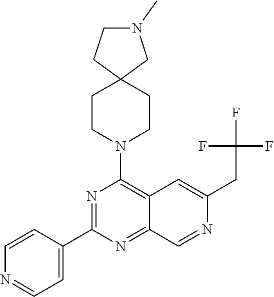 [SMILES: CN1CCC2(CCN(c3nc(-c4ccncc4)nc4cnc(CC(F)(F)F)cc34)CC2)C1]

Following the procedure described in Example 194, Steps 1-5 and making non-critical variations as required to replace tert-butyl 8-(6-formyl-2-(3-methyl-1-((2-(trimethylsilyl)ethoxy)methyl)-1H-pyrazol-4-yl)pyrido[3,4-d]pyrimidin-4-yl)-2,8-diazaspiro[4.5]decane-2-carboxylate with tert-butyl 8-(6-formyl-2-(pyridin-4-yl)pyrido[3,4-d]pyrimidin-4-yl)-2,8-diazaspiro[4.5]decane-2-carboxylate, the title compound was obtained as a yellow solid. ¹H NMR (400 MHz, DMSO-d₆) δ 9.25 (s, 1H), 8.84-8.69 (m, 2H), 8.37-8.27 (m, 2H), 7.98 (s, 1H), 4.09-3.94 (m, 4H), 3.92-3.83 (m, 2H), 2.56-2.53 (m, 2H), 2.43 (s, 2H), 2.26 (s, 3H), 1.84-1.65 (m, 6H). LCMS (ESI) m/z: 443.1 [M+H]⁺.

### Example 196

### (8-(2-(pyridin-4-yl)pyrido[3,4-d]pyrimidin-4-yl)-2,8-diazaspiro[4.5]decan-3-yl)methanol (Compound 196)

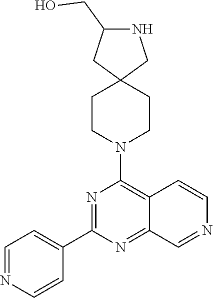 [SMILES: OCC1CC2(CCN(c3nc(-c4ccncc4)nc4cnccc34)CC2)CN1]

To a 2-dram vial was added 4-chloro-2-(4-pyridyl)pyrido[3,4-d]pyrimidine (100 mg, 0.4120 mmol), potassium fluoride (71.8 mg, 1.24 mmol, 3 equiv.), tert-butyl 3-(hydroxymethyl)-2,8-diazaspiro[4.5]decane-2-carboxylate (166 mg, 1.25 equiv., 0.515 mmol), triethylamine (0.287 mL, 2.06 mmol, 5 equiv.), and dimethyl sulfoxide (1.374 mL, 19 mmol, 0.3 M). The reaction was allowed to stir for 30 minutes at room temperature. The reaction mixture was transferred to a 20 mL vial, diluted with water (5 mL) and EtOAc (5 mL) and the layers were separated. The aqueous layer was extracted with further EtOAc (3×5 mL) and the combined organic extracts were dried over Na₂SO₄, filtered, and concentrated in vacuo. The crude residue was dissolved in 1 mL DCM and 1 mL TFA. Let stir at room temperature for 30 min. The reaction mixture was then concentrated in vacuo, and then concentrated 2× further from DCM (5 mL) to remove as much residual TFA as possible. The crude residue was purified by HPLC.

### Examples 197 and 198

### (S)-(8-(2-(pyridin-4-yl)pyrido[3,4-d]pyrimidin-4-yl)-2,8-diazaspiro[4.5]decan-3-yl)methanol and (R)-(8-(2-(pyridin-4-yl)pyrido[3,4-d]pyrimidin-4-yl)-2,8-diazaspiro[4.5]decan-3-yl)methanol (Compounds 197 and 198)

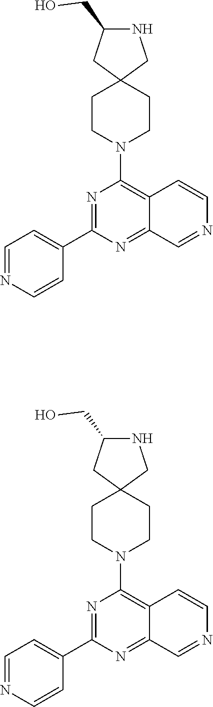 [SMILES: OC[C@@H]1CC2(CCN(c3nc(-c4ccncc4)nc4cnccc34)CC2)CN1 ; OC[C@H]1CC2(CCN(c3nc(-c4ccncc4)nc4cnccc34)CC2)CN1]

Racemic (8-(2-(pyridin-4-yl)pyrido[3,4-d]pyrimidin-4-yl)-2,8-diazaspiro[4.5]decan-3-yl)methanol was separated from the purified residue from Example 196 by using chiral SFC (Chiralpak AD (150 mm×21.2 mm, 5 um), eluting isocratic with supercritical CO₂/MeOH+0.1% NH₄OH=55/45; 70 mL/min) to give the title compounds. Absolute configuration was arbitrarily assigned to each enantiomer. Example 197 (first peak): ¹H NMR (400 MHz, DMSO-d₆) δ 9.26 (s, 1H), 8.78-8.76 (m, 2H), 8.59 (d, J=5.7 Hz, 1H), 8.35-8.30 (m, 2H), 7.90 (d, J=5.7 Hz, 1H), 4.53 (br s, 1H), 4.04-3.85 (m, 5H), 3.35 (br s, 1H), 3.25-3.16 (m, 1H), 2.75 (s, 2H), 1.88-1.61 (m, 6H), 1.30 (dd, J=12.7, 8.0 Hz, 1H). LCMS (ESI) m/z: 377.2 [M+H]⁺. Example 198 (second peak): ¹H NMR (400 MHz, DMSO-d₆) δ 9.29 (s, 1H), 8.79 (d, J=5.1 Hz, 2H), 8.62 (d, J=5.6 Hz, 1H), 8.37-8.31 (m, 2H), 7.90 (d, J=5.7 Hz, 1H), 5.46 (br s, 1H), 4.10-3.85 (m, 5H), 3.81-3.75 (m, 1H), 3.73-3.68 (m, 1H), 3.60-3.51 (m, 1H), 3.25-3.18 (m, 1H), 3.15-3.06 (m, 1H), 2.14 (dd, J=13.2, 7.4 Hz, 1H), 1.88-1.78 (m, 4H), 1.62 (dd, J=13.2, 10.0 Hz, 1H). LCMS (ESI) m/z: 377.2 [M+H]⁺.

### Example 199

### 3-(pyridin-4-yl)-1-(2,8-diazaspiro[4.5]decan-8-yl)-2,6-naphthyridine (Compound 199)

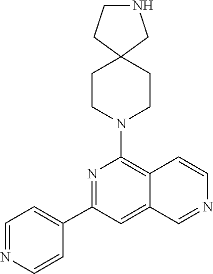 [SMILES: c1cc(-c2cc3cnccc3c(N3CCC4(CCNC4)CC3)n2)ccn1]

### Step 1: N-(tert-Butyl)-3-methylisonicotinamide

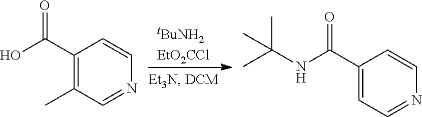

To a stirred solution of 3-methyl-4-pyridinecarboxilic acid (2.0 g, 14.6 mmol) in anhydrous DCM (55 mL) was added triethylamine (3.05 mL, 21.9 mmol) at 0° C. Ethyl chloroformate (1.7 mL, 17.8 mmol) was added slowly over 10 minutes. The resulting mixture was stirred at 0° C. for 30 minutes then tert-butylamine (1.84 mL, 17.5 mmol) was added slowly. The resulting mixture was warmed up to room temperature and stirred overnight. The reaction mixture is diluted with water (25 mL) and the dichloromethane layer was separated. The organic layer was extracted with 1 M HCl (20 mL). The aqueous layer was neutralized to pH 9 with NaOH solution. The aqueous layer is extracted twice with ethyl acetate (2×30 mL) and the combined organic layer, dried over Na₂SO₄, filtered and concentrated in vacuum to give the title compound N-tert-butyl-3-methyl-pyridine-4-carboxamide (1.7 g, 8.84 mmol, 60.6% yield) as a yellow solid. LCMS (ESI) m/z: 193.4 [M+H]⁺. ¹H NMR (400 MHz, DMSO-d₆) δ 8.45 (s, 1H), 8.42 (d, J=4.8 Hz, 1H), 8.03 (s, 1H), 7.19 (d, J=4.8 Hz, 1H), 2.27 (s, 3H), 1.35 (s, 9H).

### Step 2: N-(tert-Butyl)-3-(2-oxo-2-(pyridin-4-yl)ethyl)isonicotinamide

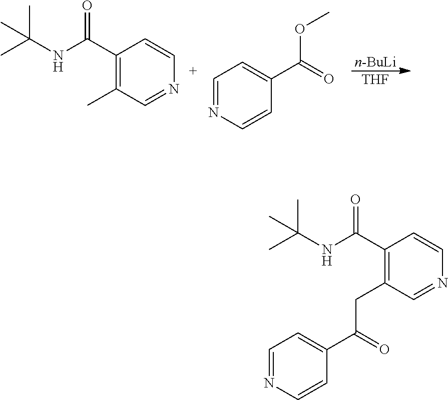

To a stirred solution of N-tert-butyl-3-methyl-pyridine-4-carboxamide (1.70 g, 8.84 mmol) in anhydrous THF (63 mL) was added n-butyllithium 2.5 M in hexane (8.5 mL, 21.2 mmol) at −45° C. over 15 minutes. The reaction was stirred at −45° C. for 45 minutes then methyl isonicotinate (1.33 mL, 9.73 mmol) in THF (5.0 mL) was slowly added via cannula. The resulting mixture was stirred at −45° C. for 30 minutes then slowly warmed up to room temperature and stirred for 2 hours. The solution was poured into a saturated aqueous NH₄Cl solution (100 ml). The organic layer was separated and the aqueous layer was extracted with EtOAc (3×75 mL). The combined organic layers were washed with saturated aqueous NaCl solution (50 mL), dried over Na₂SO₄, filtered and concentrated to give the title compound N-(tert-butyl)-3-(2-oxo-2-(pyridin-4-yl)ethyl)isonicotinamide (2.63 g, 8.85 mmol, 100% yield). LCMS (ESI) m/z: 298.1 [M+H]⁺.

### Step 3: 3-(Pyridin-4-yl)-1H-pyrano[4,3-c]pyridin-1-one

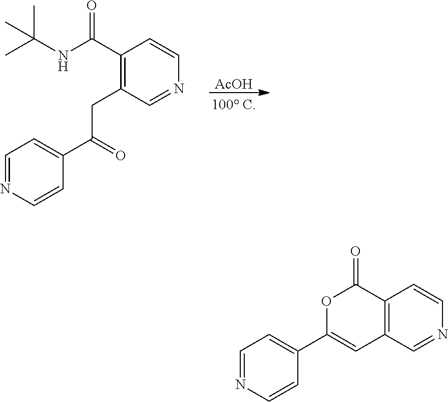 [SMILES: "AcOH" ; CC(C)(C)NC(=O)c1ccncc1CC(=O)c1ccncc1 ; O=c1oc(-c2ccncc2)cc2cnccc12]

A solution of N-(tert-butyl)-3-(2-oxo-2-(pyridin-4-yl)ethyl)isonicotinamide (2.63 g, 8.84 mmol) in acetic acid (30 mL, 525 mmol) was heated at 100° C. for 16 hours. The solution was cooled to room temperature and concentrated to ˜10 mL under reduced pressure. Water (20 mL) was added and a solid precipitated out of the solution. The mixture was filtered to get a white precipitate. The solid was washed with water and dried under reduced pressure to afford the title compound 3-(4-pyridyl)pyrano[4,3-c]pyridin-1-one (1.79 g, 7.98 mmol, 90% yield) as a white solid. LCMS (ESI) m/z: 225.2 [M+H]⁺.

### Step 4: 3-Hydroxy-3-(pyridin-4-yl)-3,4-dihydro-2,6-naphthyridin-1 (2H)-one

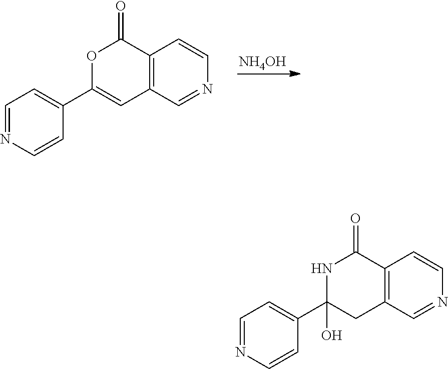

A solution of the 3-(4-pyridyl)pyrano[4,3-c]pyridin-1-one (1.79 g, 7.98 mmol) in absolute ethanol (22 mL) was added to an aqueous solution of ammonium hydroxide (17 mL, 427 mmol). The resulting mixture was stirred at room temperature for 2 hours. The crude was concentrated in vacuo to provide the title compound 3-hydroxy-3-(4-pyridyl)-2,4-dihydro-2,6-naphthyridin-1-one (1.07 g, 4.43 mmol, 55% yield) as a yellow solid. ¹H NMR (400 MHz, DMSO-d₆) δ 9.11 (s, 1H), 8.66 (d, J=4.9 Hz, 1H), 8.61 (dd, J=4.7, 1.8 Hz, 3H), 7.79 (d, J=5.0 Hz, 1H), 7.60 (dd, J=4.5, 1.7 Hz, 2H), 6.72 (s, 1H), 3.37 (d, J=16.2 Hz, 1H), 3.13 (d, J=16.1 Hz, 1H).

### Step 5: 3-(Pyridin-4-yl)-2,6-naphthyridin-1 (2H)-one

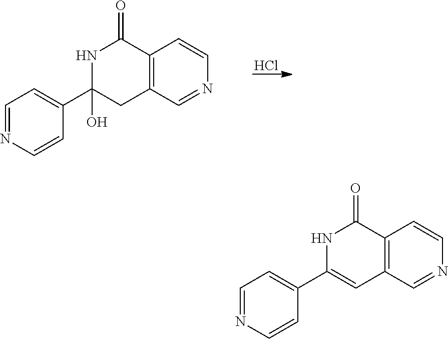 [SMILES: O=C1NC(O)(c2ccncc2)Cc2cnccc21 ; Cl ; O=c1[nH]c(-c2ccncc2)cc2cnccc12]

A solution of 3-hydroxy-3-(4-pyridyl)-2,4-dihydro-2,6-naphthyridin-1-one (1.07 g, 4.44 mmol) in absolute ethanol (20 mL) and water (2 mL) was added an aqueous solution of HCl 6 N (11 mL, 66 mmol) at 0° C. The reaction was warmed up to room temperature and stirred for 16 hours. The mixture was filtered over a Buchner. The filtrate was concentrated to give the title compound 3-(pyridin-4-yl)-2,6-naphthyridin-1 (2H)-one (955 mg, 4.27 mmol, 96% yield) as a light yellow solid. LCMS (ESI) m/z: 224.1 [M+H]⁺.

### Step 6: 1-Chloro-3-(pyridin-4-yl)-2,6-naphthyridine

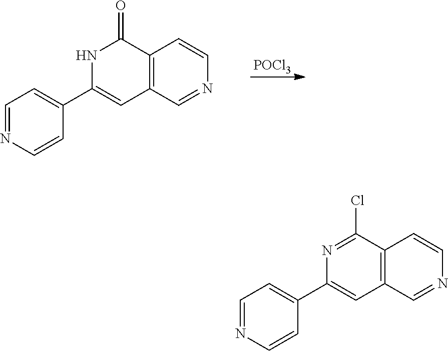

A suspension of 3-(4-pyridyl)-2H-2,6-naphthyridin-1-one (1.12 g, 5.02 mmol) in phosphorus oxychloride (8.84 mL, 94.9 mmol) was added in an open heavy wall round bottom pressure vessel (75 mL) and it was heated at 100° C. The mixture was stirred at 100° C. for 30 minutes. Then the pressure vessel was sealed, and the reaction mixture was heated at 130° C. for 15 hours. The reaction was cooled to room temperature and excess of phosphorus oxychloride was removed under reduced pressure. The residue was mixed with ice water and the pH of the mixture was adjusted to ˜ 7 with a 1M aqueous NaOH solution, and then to ˜10 with saturated aqueous Na₂CO₃ solution. Filtration of the mixture gave a pale brown solid which was dried under reduce pressure to afford the title compound 1-chloro-3-(4-pyridyl)-2,6-naphthyridine (1.01 g, 4.18 mmol, 83% yield) as a pale brown solid. LCMS (ESI) m/z: 241.9, 243.8 [M+H]⁺.

¹H NMR (400 MHz, DMSO-d₆) δ 9.60 (s, 1H), 8.94 (s, 1H), 8.90 (d, J=5.8 Hz, 1H), 8.78 (dd, J=4.5, 1.7 Hz, 2H), 8.15 (dd, J=4.6, 1.6 Hz, 3H).

### Step 7: tert-Butyl 8-(3-(pyridin-4-yl)-2,6-naphthyridin-1-yl)-2,8-diazaspiro[4.5]decane-2-carboxylate

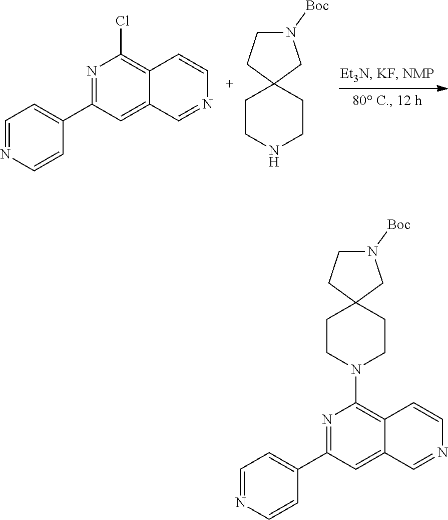

A solution of 1-chloro-3-(4-pyridyl)-2,6-naphthyridine (400 mg, 1.66 mmol), triethylamine (1.15 mL, 8.28 mmol), potassium fluoride (294 mg, 4.97 mmol), and tert-butyl 2,8-diazaspiro[4.5]decane-2-carboxylate (517 mg, 2.15 mmol) in NMP (4.00 mL) was stirred at 80° C. for 12 hours. The reaction mixture was cooled to room temperature and poured in EtOAc (50 mL) and water (25 mL). The phases were separated and the organic layer was washed with NaHCO₃ and brine (2×), dried over Na₂SO₄, filtered and concentrated with silica gel. The crude product was purified by column chromatography (MeOH/EtOAc/Heptane) to provide the title compound tert-butyl 8-(3-(pyridin-4-yl)-2,6-naphthyridin-1-yl)-2,8-diazaspiro[4.5]decane-2-carboxylate (350 mg, 47% yield) as a beige solid. LCMS (ESI) m/z: 446.1 [M+H]⁺.

### Step 8: 3-(Pyridin-4-yl)-1-(2,8-diazaspiro[4.5]decan-8-yl)-2,6-naphthyridine

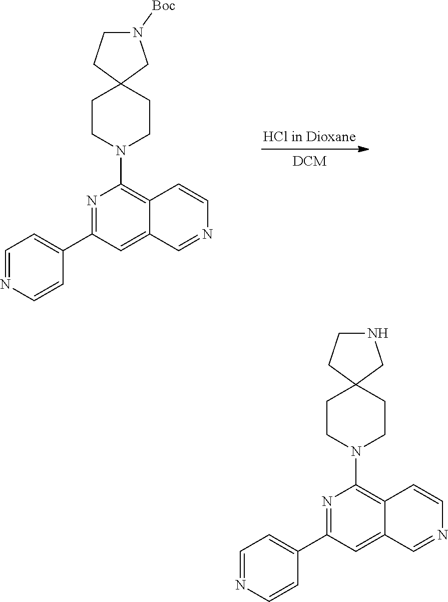 [SMILES: CC(C)(C)OC(=O)N1CCC2(CCN(c3nc(-c4ccncc4)cc4cnccc34)CC2)C1 ; c1cc(-c2cc3cnccc3c(N3CCC4(CCNC4)CC3)n2)ccn1]

To a solution of tert-butyl 8-(3-(pyridin-4-yl)-2,6-naphthyridin-1-yl)-2,8-diazaspiro[4.5]decane-2-carboxylate (47 mg, 0.110 mmol) in DCM (0.50 mL) was added HCl 4 N in dioxane (1.0 mL, 4.0 mmol) in DCM (0.5 mL). After the addition of HCl was completed a yellow precipitate was formed. The reaction was stirred at room temperature for 15 minutes. The reaction was then concentrated to dryness. The crude was redissolved in MeCN and water, freezed and lyophilized to give the title compound 3-(pyridin-4-yl)-1-(2,8-diazaspiro[4.5]decan-8-yl)-2,6-naphthyridine hydrochloride (35 mg, 0.092 mmol, 87% yield) as a orange solid. LCMS (ESI) m/z: 346.3 [M+H]⁺. ¹H NMR (400 MHz, DMSO-d₆) δ 9.44 (s, 1H), 9.16 (br s, 2H), 9.00-8.91 (m, 2H), 8.75 (d, J=5.8 Hz, 1H), 8.65-8.53 (m, 2H), 7.94 (d, J=5.8 Hz, 1H), 3.65-3.53 (m, 4H), 3.35-3.24 (m, 2H), 3.12 (t, J=5.5 Hz, 2H), 1.99-1.78 (m, 6H).

### Example 200

### 2-(Pyridin-4-yl)-4-(2,8-diazaspiro[4.5]decan-8-yl)-1,7-naphthyridine (Compound 200)

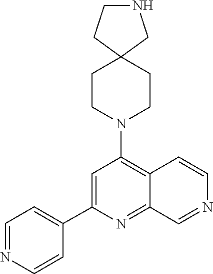 [SMILES: c1cc(-c2cc(N3CCC4(CCNC4)CC3)c3ccncc3n2)ccn1]

### Step 1: 4-Hydroxy-1,7-naphthyridin-2 (1H)-one

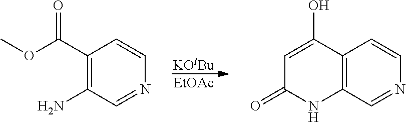

To a stirred solution of methyl 3-aminopyridine-4-carboxylate (5.0 g, 32.9 mmol) in anhydrous ethyl acetate (32 mL, 328 mmol) under nitrogen was added potassium tert-butoxide (7.74 g, 69.0 mmol). The resulting mixture was stirred at 75° C. for 14 hours then for 3 hours at 80° C. The reaction mixture was cooled to room temperature and 100 mL of water were added. The organic layer was separated and the aqueous layer was washed with ethyl acetate and twice with tert-butyl methyl ether. The aqueous layer was acidified with 6 N HCl to a pH of 6 which precipitated a solid. The resulting precipitate was filtered off, washed with water and dried under vacuum. To the wet solid was added small amount of water and the slurry was frozen and lyophilized to give the title compound 4-hydroxy-1H-1,7-naphthyridin-2-one (1.17 g, 7.22 mmol, 22% yield) as a beige solid. ¹H NMR (400 MHz, DMSO-d₆) δ 11.79 (s, 1H), 11.44 (s, 1H), 8.61 (s, 1H), 8.29 (d, J=5.2 Hz, 1H), 7.64 (d, J=5.2 Hz, 1H), 5.88 (s, 1H).

### Step 2: 2,4-dichloro-1,7-naphthyridine

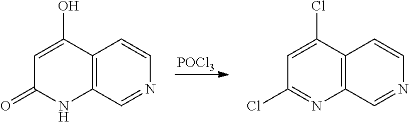

To a stirred solution of 4-hydroxy-1H-1,7-naphthyridin-2-one (1.17 g, 7.22 mmol) in toluene (14.4 mL) was added phosphorus oxychloride (3.4 mL, 36.5 mmol). The resulting mixture was stirred at 80° C. for 20 hours. The reaction was cooled to room temperature and evaporated to dryness. An aqueous solution of 1 M NaOH (50 mL) and dichloromethane (100 mL) were added. The two layers were separated and the aqueous layer was extracted with DCM (3×100 mL), dried over Na₂SO₄, filtered and evaporated to give the title compound 2,4-dichloro-1,7-naphthyridine (880 mg, 4.42 mmol, 61% yield) as a beige solid. LCMS (ESI) m/z: 199.3, 201.3 [M+H]⁺. ¹H NMR (400 MHz, DMSO-d₆) δ 9.41 (s, 1H), 8.81 (d, J=5.7 Hz, 1H), 8.28 (s, 1H), 8.08 (d, J=5.7 Hz, 1H).

### Step 3: 4-Chloro-2-(pyridin-4-yl)-1,7-naphthyridine

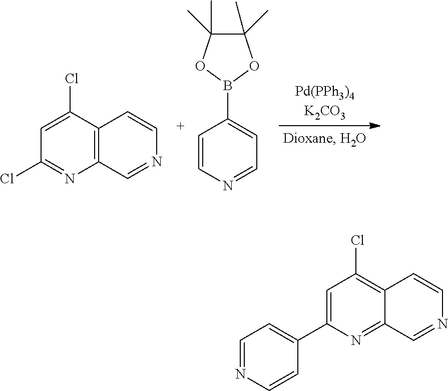

A mixture of 2,4-dichloro-1,7-naphthyridine (400 mg, 2.01 mmol), pyridine-4-boronic acid pinacol ester (433 mg, 2.11 mmol) and K₂CO₃ (833 mg, 6.03 mmol) was added in a mixture of 1,4-Dioxane (10.7 mL) and Water (2.7 mL). The solution was degassed for 5 minutes with a stream of nitrogen before Pd(PPh₃)₄ (232.2 mg, 0.20 mmol) was added. The reaction was then heated to 150° C. in a microwave apparatus and stirred for 25 minutes. The reaction was cooled to room temperature and diluted with EtOAc (50 mL). Na₂SO₄ was added and the suspension was filtrated on celite. The cake was rinsed with EtOAc and the filtrate was concentrated to dryness. The crude product was purified by column chromatography (MeOH/EtOAc/Heptane) to provide the title compound 4-chloro-2-(4-pyridyl)-1,7-naphthyridine (330 mg, 1.37 mmol, 68% yield) as a yellow solid. LCMS (ESI) m/z: 242.2, 244.1 [M+H]⁺. ¹H NMR (400 MHz, DMSO-d₆) δ 9.58 (s, 1H), 8.82 (dd, J=7.9, 3.2 Hz, 4H), 8.34-8.27 (m, 2H), 8.11 (d, J=5.7 Hz, 1H).

### Step 4: tert-Butyl 8-(2-(pyridin-4-yl)-1,7-naphthyridin-4-yl)-2,8-diazaspiro[4.5]decane-2-carboxylate

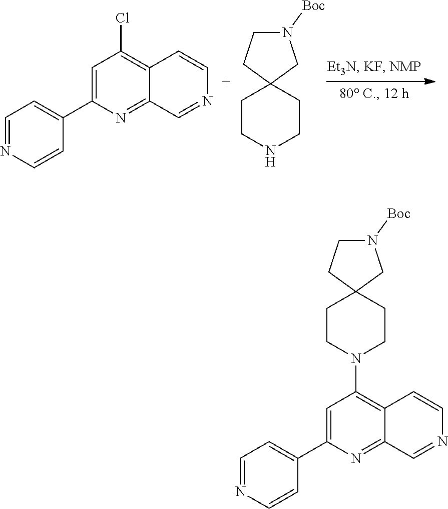

A solution of 4-chloro-2-(4-pyridyl)-1,7-naphthyridine (180 mg, 0.740 mmol), triethylamine (0.52 mL, 3.72 mmol), potassium fluoride (132 mg, 2.23 mmol), and tert-butyl 2,8-diazaspiro[4.5]decane-2-carboxylate (233 mg, 0.970 mmol) in NMP (3.72 mL) was stirred at 80° C. for 12 hours. The reaction mixture was cooled to room temperature and poured in EtOAc (50 mL) and water (25 mL). The phases were separated and the organic layer was washed with NaHCO₃ and brine (2×), dried over Na₂SO₄, filtered and concentrated with silica gel. The crude product was purified by column chromatography (MeOH/EtOAc/Heptane) to provide the title compound tert-butyl 8-(2-(pyridin-4-yl)-1,7-naphthyridin-4-yl)-2,8-diazaspiro[4.5]decane-2-carboxylate (295 mg, 89% yield) as a clear oil. LCMS (ESI) m/z: 446.0 [M+H]⁺.

### Step 5: 2-(Pyridin-4-yl)-4-(2,8-diazaspiro[4.5]decan-8-yl)-1,7-naphthyridine

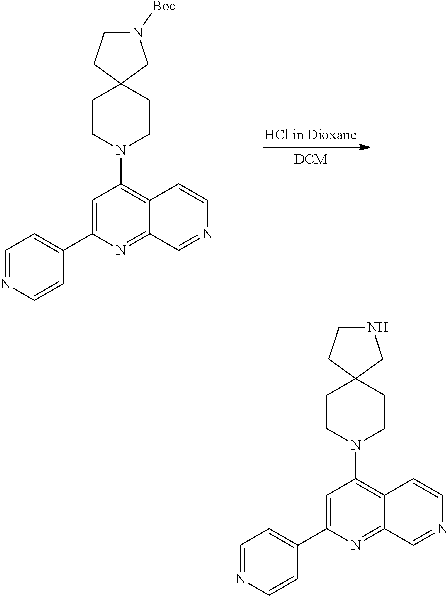 [SMILES: CC(C)(C)OC(=O)N1CCC2(CCN(c3cc(-c4ccncc4)nc4cnccc34)CC2)C1 ; c1cc(-c2cc(N3CCC4(CCNC4)CC3)c3ccncc3n2)ccn1]

To a solution of tert-butyl 8-[2-(4-pyridyl)-1,7-naphthyridin-4-yl]-2,8-diazaspiro[4.5]decane-2-carboxylate (121 mg, 0.270 mmol) in DCM (0.80 mL) was added HCl 4 N in dioxane (0.80 mL, 3.2 mmol). After the addition of HCl was completed a yellow precipitate was formed. The reaction was stirred at room temperature for 15 minutes. The reaction was then concentrated to dryness. The product was purified by reverse chromatography over a C18 column (MeCN/10 mM ammonium formate pH 3.8 aqueous buffer). The fractions containing the product were combined and concentrated to remove some MeCN, freezed and lyophilized to give the title compound 2-(pyridin-4-yl)-4-(2,8-diazaspiro[4.5]decan-8-yl)-1,7-naphthyridine (75 mg, 0.217 mmol, 80% yield) as a pale orange solid. ¹H NMR (400 MHz, DMSO-d₆) δ 9.39 (s, 1H), 8.78 (d, J=5.8 Hz, 2H), 8.57 (d, J=5.7 Hz, 1H), 8.39 (br s, 1H), 8.24 (d, J=5.8 Hz, 2H), 7.84 (d, J=5.7 Hz, 1H), 7.72 (s, 1H), 3.42-3.30 (m, 4H), 3.22-3.09 (m, 2H), 3.03-2.88 (m, 2H), 1.92-1.75 (m, 6H). LCMS (ESI) m/z: 346.3 [M+H]⁺.

### Example 201

### 2-(pyrimidin-4-yl)-4-(2,8-diazaspiro[4.5]decan-8-yl)-1,7-naphthyridine trifluoroacetate (Compound 201)

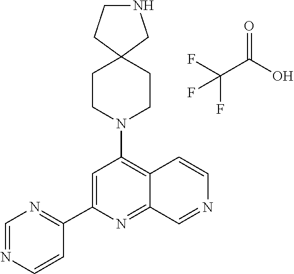

Following the procedure described in Example 101 and making non-critical variations as required to replace 4-chloro-2-(pyridin-4-yl)pyrido[3,4-d]pyrimidine with 4-chloro-2-(pyrimidin-4-yl)-1,7-naphthyridine (prepared according to the procedure in WO2018198077), the title compound was obtained as a yellow solid. ¹H NMR (400 MHz, DMSO-d₆) δ 9.44 (s, 1H), 9.40 (s, 1H), 9.06 (d, J=5.2 Hz, 1H), 8.61 (d, J=5.6 Hz, 1H), 8.57 (d, J=5.2 Hz, 1H), 8.29 (s, 1H), 8.20 (s, 1H), 7.88 (d, J=5.2 Hz, 1H), 3.47-3.33 (m, 4H), 3.29-3.23 (m, 2H), 3.08 (s, 2H), 1.93-1.84 (m, 6H). LCMS (ESI) m/z: 347.0 [M+H].

### Example 202 tert-butyl 8-(2-(pyridin-4-yl)pyrido[3,4-d]pyrimidin-4-yl)-2,8-diazaspiro[4.5]decane-2-carboxylate (Compound 202)

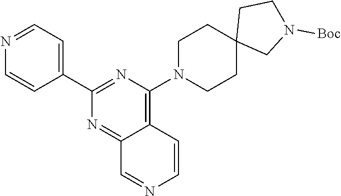 [SMILES: CC(C)(C)OC(=O)N1CCC2(CCN(c3nc(-c4ccncc4)nc4cnccc34)CC2)C1]

To a solution of 4-chloro-2-(pyridin-4-yl)pyrido[3,4-d]pyrimidine (3.0 g, 12.36 mmol) in DMF (50 mL) was added tert-butyl 2,8-diazaspiro[4.5]decane-2-carboxylate (2.9 g, 12.36 mmol), triethylamine (8.6 mL, 61.81 mmol) and potassium fluoride (0.7 g, 12.36 mmol). The mixture was heated to 80° C. for 16 h under nitrogen atmosphere. After cooling to room temperature, the reaction was diluted with water (200 mL) and extracted with EtOAc (400 mL). The organic layer was washed with brine (200 mL×3), dried over anhydrous Na₂SO₄, filtered and concentrated in vacuo. The crude residue was purified by silica gel chromatography (solvent gradient: 0-5% MeOH in DCM) to give the title compound (1.6 g, 82%) as a yellow solid. ¹H NMR (400 MHz, DMSO-d₆) δ 9.26 (s, 1H), 8.81-8.73 (m, 2H), 8.59 (d, J=5.6 Hz, 1H), 8.36-8.29 (m, 2H), 7.90 (d, J=5.6 Hz, 1H), 4.03-3.93 (m, 4H), 3.41-3.35 (m, 2H), 3.22-3.20 (m, 2H), 1.85-1.81 (m, 2H), 1.79-1.69 (m, 4H), 1.41 (s, 9H). LCMS (ESI) m/z: 447.2 [M+H]⁺.

### Example 203

### 8-methoxy-4-(2-methyl-2,8-diazaspiro[4.5]decan-8-yl)-2-(pyridin-4-yl)pyrido[3,4-d]pyrimidine (Compound 203) formate

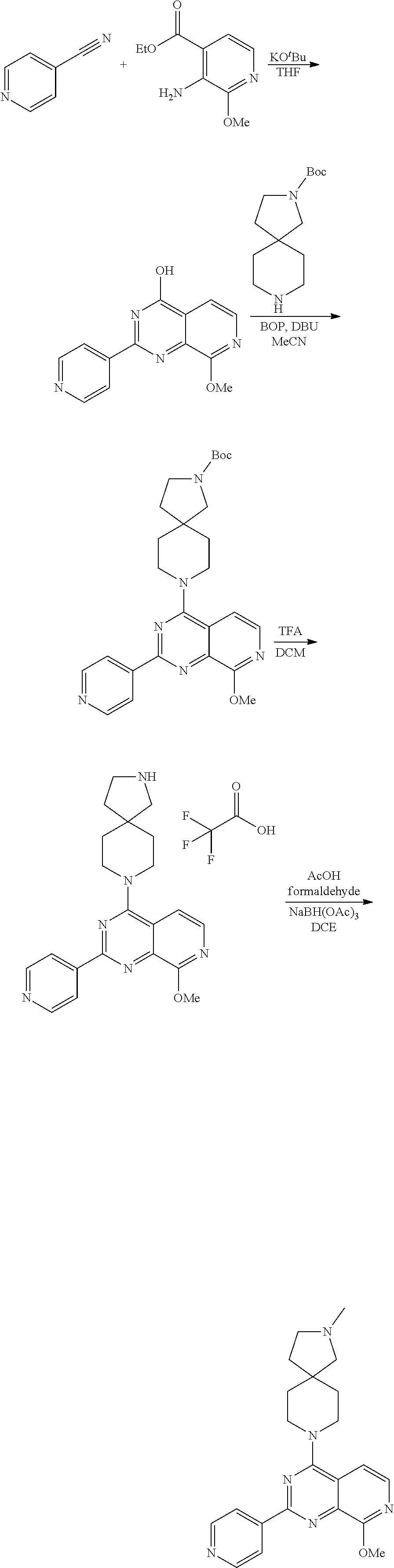

### Step 1: 8-methoxy-2-(pyridin-4-yl)pyrido[3,4-d]pyrimidin-4-ol

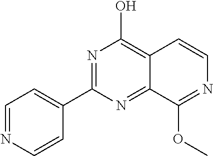 [SMILES: COc1nccc2c(O)nc(-c3ccncc3)nc12]

Following the procedure described in Example 101, Step 1 and making non-critical variations as required to replace methyl 3-aminoisonicotinate with methyl 3-amino-2-methoxyisonicotinate, the title compound was obtained (850 mg, 61%) as a yellow solid. LCMS (ESI) m/z: 255.2 [M+H]⁺.

### Step 2: tert-butyl 8-(8-methoxy-2-(pyridin-4-yl)pyrido[3,4-d]pyrimidin-4-yl)-2,8-diazaspiro[4.5]decane-2-carboxylate

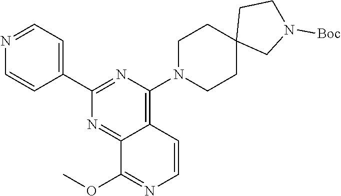 [SMILES: COc1nccc2c(N3CCC4(CCN(C(=O)OC(C)(C)C)C4)CC3)nc(-c3ccncc3)nc12]

A mixture of 8-methoxy-2-(pyridin-4-yl)pyrido[3,4-d]pyrimidin-4-ol (0.9 g, 3.54 mmol), BOP (1.88 g, 4.25 mmol) and DBU (0.81 g, 5.31 mol) in acetonitrile (10 mL) was stirred at room temperature for 10 min before tert-butyl 2,8-diazaspiro[4.5]decane-2-carboxylate (1.02 g, 4.25 mmol) was added. The reaction was stirred at room temperature for 16 h. The reaction mixture was quenched with water (50 mL), extracted with EtOAc (80 mL×2). The combined organic layers were washed with water (50 mL) and brine (50 mL), dried over anhydrous Na₂SO₄ and concentrated in vacuo. The crude residue was purified by flash chromatography (solvent gradient: 0-3% MeOH in DCM) to give the title compound (755 mg, 45%) as a yellow solid. LCMS (ESI) m/z: 477.3 [M+H]⁺.

### Step 3: 8-methoxy-2-(pyridin-4-yl)-4-(2,8-diazaspiro[4.5]decan-8-yl)pyrido[3,4-d]pyrimidine trifluoro acetate

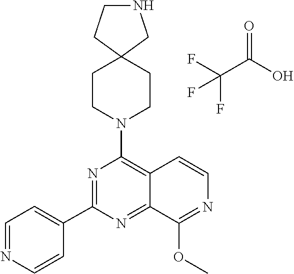

To a solution of tert-butyl 8-(8-methoxy-2-(pyridin-4-yl)pyrido[3,4-d]pyrimidin-4-yl)-2,8-diazaspiro[4.5]decane-2-carboxylate (610 mg, 1.28 mmol) in DCM (6 mL) was added trifluoroacetic acid (2 mL, 25.96 mmol). The mixture was stirred at room temperature for 2 h. The mixture was concentrated in vacuo to give the title compound (480 mg, crude) as brown oil that required no further purification. LCMS (ESI) m/z: 377.4 [M+H]⁺

### Step 4: 8-methoxy-4-(2-methyl-2,8-diazaspiro[4.5]decan-8-yl)-2-(pyridin-4-yl)pyrido[3,4-d]pyrimidine

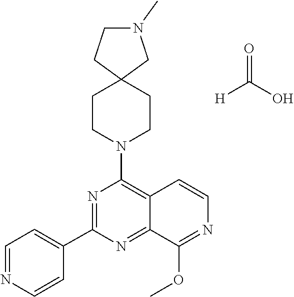 [SMILES: COc1nccc2c(N3CCC4(CCN(C)C4)CC3)nc(-c3ccncc3)nc12 ; O=CO]

Following the procedure described in Example 102 and making non-critical variations as required to replace 4-(2,8-diazaspiro[4.5]decan-8-yl)-2-(4-pyridyl)pyrido[3,4-d]pyrimidine hydrochloride with 8-methoxy-2-(pyridin-4-yl)-4-(2,8-diazaspiro[4.5]decan-8-yl)pyrido[3,4-d]pyrimidine trifluoroacetate, the title compound was obtained (140 mg, 40%) as a yellow solid. ¹H NMR (400 MHz, DMSO-d₆) δ 10.05 (br s, 1H), 8.77 (d, J=5.6 Hz, 2H), 8.30 (d, J=5.6 Hz, 2H), 8.11 (d, J=5.6 Hz, 1H), 7.39 (d, J=5.6 Hz, 1H), 4.08 (s, 3H), 4.00-3.89 (m, 2H), 3.85-3.75 (m, 2H), 3.70-3.53 (m, 2H), 3.24-3.11 (m, 1H), 3.00-2.91 (m, 1H), 2.88 (s, 3H), 2.17-2.00 (m, 1H), 1.98-1.75 (m, 5H). LCMS (ESI) m/z: 391.4 [M+H]⁺.

### Example 204

### 5-Ethyl-2-(pyridin-4-yl)-4-(2,8-diazaspiro[4.5]decan-8-yl)pyrido[3,4-d]pyrimidine (Compound 204) formate

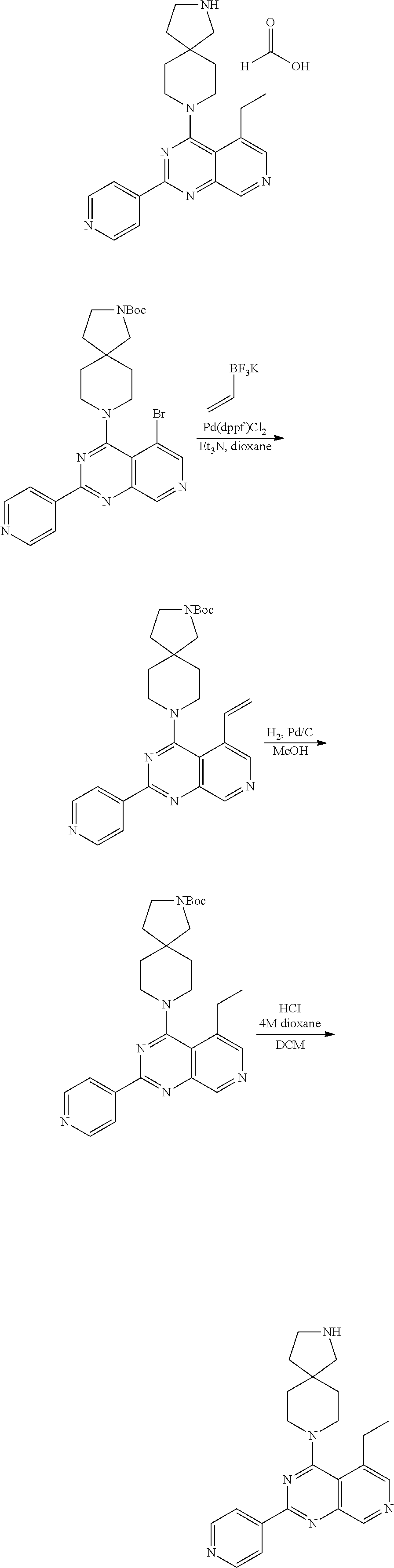

### Step 1: tert-Butyl 8-(2-(pyridin-4-yl)-5-vinylpyrido[3,4-d]pyrimidin-4-yl)-2,8-diazaspiro[4.5]decane-2-carboxylate

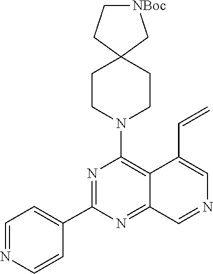 [SMILES: C=Cc1cncc2nc(-c3ccncc3)nc(N3CCC4(CCN(C(=O)OC(C)(C)C)C4)CC3)c12]

tert-Butyl 8-[5-bromo-2-(4-pyridyl)pyrido[3,4-d]pyrimidin-4-yl]-2,8-diazaspiro[4.5]decane-2-carboxylate (127 mg, 0.24 mmol) and potassium vinyltrifluoroborate (35.6 mg, 0.27 mmol) were dissolved in 1,4-dioxane (3 mL) and degassed with nitrogen flow for 10 minutes. Triethylamine (0.07 mL, 0.48 mmol) was added while the solution was degasing for another 5 minutes. Then, [1,1′-bis(diphenylphosphino)ferrocene]dichloropalladium(ll) (17.7 mg, 0.02 mmol) was added to the reaction mixture and it was capped under nitrogen and heated to 85° C. for 6 hours. The reaction mixture was cooled to 23° C. and filtered through Celite. The filtrate was diluted a saturated solution of sodium bicarbonate and it was extracted 3 times with 2:8 iPrOH/CHCl₃. The organic layers were combined, dried with anhydrous sodium sulfate, filtered and concentrated to dryness. Crude residue was purified by flash chromatography on silica gel (SiO₂, MeOH/DCM) to provide tert-butyl 8-[2-(4-pyridyl)-5-vinyl-pyrido[3,4-d]pyrimidin-4-yl]-2,8-diazaspiro[4.5]decane-2-carboxylate as an orange solid (59 mg, 0.124 mmol, 52% yield). UPLCMS (ESI) m/z: 473.3 [M+H]⁺.

### Step 2: tert-Butyl 8-(5-ethyl-2-(pyridin-4-yl)pyrido[3,4-d]pyrimidin-4-yl)-2,8-diazaspiro[4.5]decane-2-carboxylate

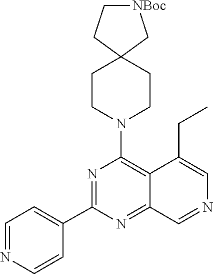 [SMILES: CCc1cncc2nc(-c3ccncc3)nc(N3CCC4(CCN(C(=O)OC(C)(C)C)C4)CC3)c12]

tert-Butyl 8-[2-(4-pyridyl)-5-vinyl-pyrido[3,4-d]pyrimidin-4-yl]-2,8-diazaspiro[4.5]decane-2-carboxylate (58 mg, 0.12 mmol) was dissolved in methanol (3 mL) and degassed with nitrogen flow for 10 minutes prior to addition of the 10% wt palladium on carbon (6 mg). The reaction mixture was capped and hydrogen was introduce through bubbling for 5 minutes. Reaction mixture was stirred at 23° C. under 1 atm hydrogen (balloon) for 1 hour. The reaction mixture was purged with nitrogen, filtered through Celite® and rinse with MeOH. The filtrate was concentrated under reduced pressure to provide tert-butyl 8-[5-ethyl-2-(4-pyridyl)pyrido[3,4-d]pyrimidin-4-yl]-2,8-diazaspiro[4.5]decane-2-carboxylate as a beige solid (58 mg, 0.12 mmol, 99% yield). UPLCMS (ESI) m/z: 475.2 [M+H].

### Step 3: 5-Ethyl-2-(pyridin-4-yl)-4-(2,8-diazaspiro[4.5]decan-8-yl)pyrido[3,4-d]pyrimidine formate

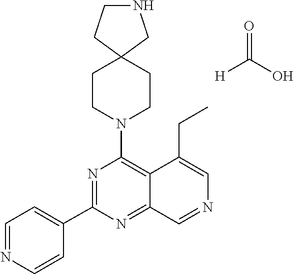

Following the procedure described in Example 199, Step 8 and making non-critical variations as required to replace the substrate with tert-butyl 8-(5-ethyl-2-(pyridin-4-yl)pyrido[3,4-d]pyrimidin-4-yl)-2,8-diazaspiro[4.5]decane-2-carboxylate was obtained the titled compound as an off white solid (16 mg, 0.041 mmol, 34% yield). ¹H NMR (400 MHz, DMSO-d₆) δ 9.09 (s, 1H), 8.78 (d, J=5.1 Hz, 2H), 8.56 (s, 1H), 8.38 (s, 1H), 8.32 (d, J=5.2 Hz, 2H), 3.75-3.61 (m, 4H), 3.18-3.04 (m, 6H), 2.54 (s, 2H), 1.96-1.84 (m, 1H), 1.76-1.57 (m, 5H), 1.19 (t, J=7.4 Hz, 3H). UPLCMS (ESI) m/z: 375.3 [M+H]⁺.

### Example 205

### 1-((8-(2-(Pyridin-4-yl)pyrido[3,4-d]pyrimidin-4-yl)-2,8-diazaspiro[4.5]decan-2-yl)methyl)cyclobutan-1-ol (Compound 205) formate

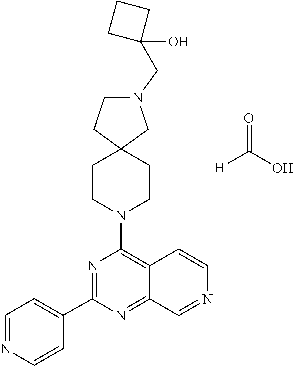 [SMILES: OC1(CN2CCC3(CCN(c4nc(-c5ccncc5)nc5cnccc45)CC3)C2)CCC1 ; O=CO]

A solution of 4-(2,8-diazaspiro[4.5]decan-8-yl)-2-(4-pyridyl)pyrido[3,4-d]pyrimidine, hexahydrochloride (200 mg, 0.35 mmol), 1-oxaspiro[2.3]hexane (45 mg, 0.53 mmol), triethylamine (390 μL, 2.83 mmol) in MeOH (1.8 mL) and DCM (1.8 mL) was heated and stirred at 50° C. After 20 hours, the solvent was removed under reduced pressure. The crude was purified by reverse phase chromatography using C18 (MeCN/Ammonium formate pH 3.7 from 0% to 60%) to afford 1-[[8-[2-(4-pyridyl)pyrido[3,4-d]pyrimidin-4-yl]-2,8-diazaspiro[4.5]decan-2-yl]methyl]cyclobutanol (42 mg, 0.088 mmol, 25% yield) as a dark green solid (formate salt).

¹H NMR (400 MHz, DMSO-d₆) 9.24 (s, 1H), 8.75 (d, J=5.9 Hz, 2H), 8.57 (d, J=5.7 Hz, 1H), 8.31 (d, J=5.9 Hz, 2H), 8.28 (s, 1H), 7.88 (d, J=5.7 Hz, 1H), 4.03-3.93 (m, 2H), 3.91-3.80 (m, 2H), 2.68 (t, J=6.9 Hz, 2H), 2.50-2.47 (m, 4H), 2.04-1.95 (m, 2H), 1.94-1.83 (m, 2H), 1.80-1.71 (m, 4H), 1.69-1.54 (m, 3H), 1.50-1.32 (m, 1H). LCMS (ESI) m/z: 431.3 [M+H]⁺.

### Example 206

### (2-cyclopentyl-8-(2-(pyridin-4-yl)pyrido[3,4-d]pyrimidin-4-yl)-2,8-diazaspiro[4.5]decan-3-yl)methanol (Compound 206)

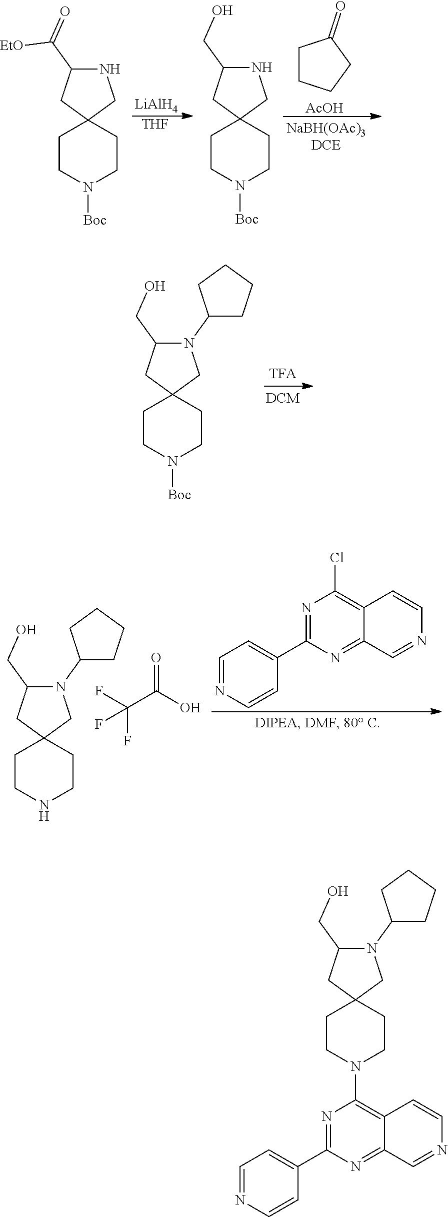

### Step 1: tert-butyl 3-(hydroxymethyl)-2,8-diazaspiro[4.5]decane-8-carboxylate

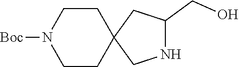 [SMILES: CC(C)(C)OC(=O)N1CCC2(CC1)CNC(CO)C2]

To a solution of 8-tert-butyl 3-ethyl 2,8-diazaspiro[4.5]decane-3,8-dicarboxylate (100 mg, 0.32 mmol) (prepared according to the procedure in WO201887602) in THF (2 mL) was added lithium aluminum hydride (18.2 mg, 0.48 mmol) slowly at 0° C. Then the reaction mixture was stirred at 0° C. for 1 h. The mixture was quenched by water (0.1 mL), 1M aq. NaOH (0.1 mL), water (0.1 mL), diluted with EtOAc (30 mL), and then dried over anhydrous MgSO₄. The mixture was filtered and filter cake was washed with EtOAc (10 mL×2). The filtrate was concentrated in vacuo to give the title compound (80 mg, crude) as yellow oil that required no further purification. LCMS (ESI) m/z: 271.2 [M+H].

### Step 2: tert-butyl 2-cyclopentyl-3-(hydroxymethyl)-2,8-diazaspiro[4.5]decane-8-carboxylate

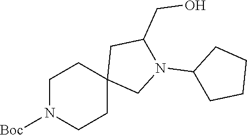 [SMILES: CC(C)(C)OC(=O)N1CCC2(CC1)CC(CO)N(C1CCCC1)C2]

To a solution of tert-butyl 3-(hydroxymethyl)-2,8-diazaspiro[4.5]decane-8-carboxylate (80 mg, 0.30 mmol) in 1,2-dichloroethane (2 mL) was added cyclopentanone (80 uL, 0.89 mmol) and acetic acid (20 uL, 0.30 mmol). The mixture was stirred at room temperature for 10 min before the addition of sodium triacetoxyborohydride (188 mg, 0.89 mmol). The mixture was stirred at room temperature for 16 h. The reaction mixture was concentrated in vacuo. The crude residue was dissolved in EtOAc (30 mL), washed with sat. aq. NaHCO₃ (15 mL) and brine (15 mL). The organic layer was dried over anhydrous Na₂SO₄, filtered and concentrated in vacuo. The crude residue was purified by Prep-TLC (DCM/MeOH=20:1) to give the title compound (40 mg, 40%) as a white solid. LCMS (ESI) m/z: 339.3 [M+H]⁺.

### Step 3: (2-cyclopentyl-2,8-diazaspiro[4.5]decan-3-yl)methanol trifluoroacetate

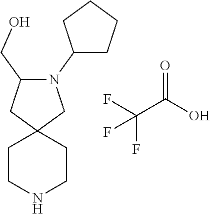

To a solution of tert-butyl 2-cyclopentyl-3-(hydroxymethyl)-2,8-diazaspiro[4.5]decane-8-carboxylate (40 mg, 0.12 mmol) in DCM (1 mL) was added trifluoroacetic acid (0.2 mL, 2.6 mmol). The mixture was stirred at room temperature for 2 h. The mixture was concentrated in vacuo to give the title compound (40 mg, crude) as yellow oil that required no further purification. LCMS (ESI) m/z: 239.2 [M+H]⁺

### Step 4: (2-cyclopentyl-8-(2-(pyridin-4-yl)pyrido[3,4-d]pyrimidin-4-yl)-2,8-diazaspiro[4.5]decan-3-yl)methanol

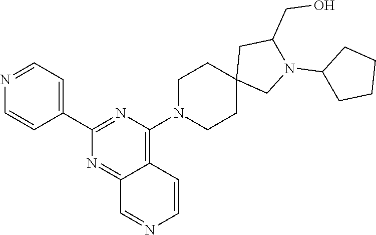 [SMILES: OCC1CC2(CCN(c3nc(-c4ccncc4)nc4cnccc34)CC2)CN1C1CCCC1]

To a solution of 4-chloro-2-(pyridin-4-yl)pyrido[3,4-d]pyrimidine (25 mg, 0.10 mmol) in DMF (1 mL) was added (2-cyclopentyl-2,8-diazaspiro[4.5]decan-3-yl)methanol trifluoroacetate (36 mg, 0.10 mmol) and N,N-diisopropylethylamine (89 uL, 0.52 mmol). The mixture was heated to 80° C. for 16 h under nitrogen atmosphere. After cooling to room temperature, the resulting mixture was purified by reverse phase chromatography (acetonitrile 27-57%/0.05% NH₃·H₂O+10 mM NH₄HCO₃ in water) to give the title compound (9 mg, 20%) as a yellow solid. ¹H NMR (400 MHz, DMSO-d₆) δ 9.26 (s, 1H), 8.83-8.72 (m, 2H), 8.58 (d, J=5.6 Hz, 1H), 8.37-8.30 (m, 2H), 7.90 (d, J=5.6 Hz, 1H), 4.42 (t, J=5.6 Hz, 1H), 4.08-3.85 (m, 4H), 3.57-3.50 (m, 1H), 3.27-3.19 (m, 1H), 3.02-2.91 (m, 2H), 2.81-2.72 (m, 1H), 2.40-2.36 (m, 1H), 1.83-1.64 (m, 8H), 1.62-1.33 (m, 6H). LCMS (ESI) m/z: 445.2 [M+H]⁺.

### Example 207

### 2-(pyridin-4-yl)-4-(2,8-diazaspiro[4.5]decan-8-yl)pyrido[3,4-d]pyrimidine 7-oxide (Compound 207) trifluoroacetate salt

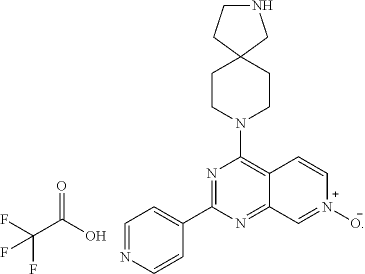

### Step 1: 4-(2-(tert-butoxycarbonyl)-2,8-diazaspiro[4.5]decan-8-yl)-2-chloropyrido[3,4-d]pyrimidine 7-oxide

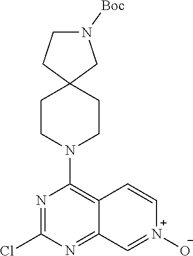 [SMILES: CC(C)(C)OC(=O)N1CCC2(CCN(c3nc(Cl)nc4c[n+]([O-])ccc34)CC2)C1]

To a solution of tert-butyl 8-(2-chloropyrido[3,4-d]pyrimidin-4-yl)-2,8-diazaspiro[4.5]decane-2-carboxylate (300 mg, 0.74 mmol) in DCM (8 mL) was added 3-chloroperoxybenzoicacid (192 mg, 0.89 mmol). The mixture was stirred at room temperature for 3 h. The reaction mixture was diluted with water (30 mL), extracted with DCM (30 mL×2). The combined organic layers were dried over anhydrous Na₂SO₄, filtered and concentrated in vacuo. The crude residue was purified by column chromatography (solvent gradient: 0-5% MeOH in DCM) to give the title compound (230 mg, 74%) as a yellow solid. LCMS (ESI) m/z: 420.3 [M+H]⁺.

### Step 2: 2-(pyridin-4-yl)-4-(2,8-diazaspiro[4.5]decan-8-yl)pyrido[3,4-d]pyrimidine 7-oxide trifluoroacetate salt

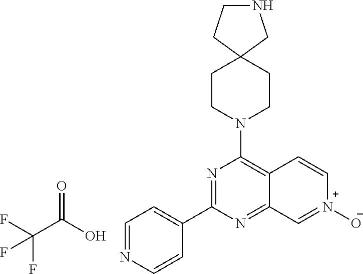

Following the procedure described in Example 153, Step 2 and making non-critical variations as required to replace tert-butyl 8-(2-chloropyrido[3,4-d]pyrimidin-4-yl)-2,8-diazaspiro[4.5]decane-2-carboxylate and 3-chloro-4-(4,4,5,5-tetramethyl-1,3,2-dioxaborolan-2-yl)-1H-pyrazole with 4-(2-(tert-butoxycarbonyl)-2,8-diazaspiro[4.5]decan-8-yl)-2-chloropyrido[3,4-d]pyrimidine 7-oxide and pyridin-4-ylboronic acid, the title compound was obtained (64 mg, 30%) as a yellow solid. ¹H NMR (400 MHz, DMSO-d₆) δ 8.89 (s, 1H), 8.80-8.77 (m, 2H), 8.75 (d, J=2.0 Hz, 1H), 8.31-8.28 (m, 2H), 8.20-8.15 (m, 1H), 7.94 (d, J=7.2 Hz, 1H), 4.00-3.87 (m, 4H), 3.32-3.27 (m, 2H), 3.11 (t, J=5.6 Hz, 2H), 1.93 (t, J=7.6 Hz, 2H), 1.82-1.75 (m, 4H). LCMS (ESI) m/z: 363.3 [M+H]⁺.

### Example 208

### 4-(2-cyclopentyl-2,8-diazaspiro[4.5]decan-8-yl)-2-(1,3-dimethyl-1H-pyrazol-4-yl)pyrido[3,4-d]pyrimidine (Compound 208)

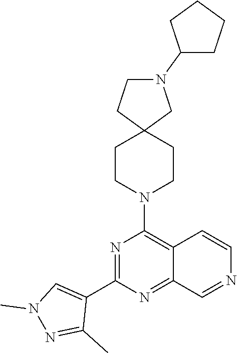 [SMILES: Cc1nn(C)cc1-c1nc(N2CCC3(CC2)CCN(C2CCCC2)C3)c2ccncc2n1]

To a solution of 4-(2-cyclopentyl-2,8-diazaspiro[4.5]decan-8-yl)-2-(3-methyl-1H-pyrazol-4-yl)pyrido[3,4-d]pyrimidine (220 mg, 0.52 mmol) in THF (5 mL) was added NaH (39 mg, 0.98 mmol, 60%) at 0° C. under nitrogen atmosphere. After 10 min, iodomethane (42 uL, 0.73 mmol) was added. The reaction was stirred at 0° C. for 2 h. The reaction was poured into sat. aq. NH₄Cl (5 mL) and water (10 mL), extracted with EtOAc (30 mL×2). The combined organic layers were washed with brine (30 mL), dried over anhydrous Na₂SO₄, filtered and concentrated in vacuo. The residue was purified by reverse phase chromatography (acetonitrile 15-45%/0.225% formic acid in water) to give a mixture (positional isomer) (55 mg, 41%) as yellow solid. The mixture was separated by using chiral SFC (Chiralpak AD (250 mm*30 mm, 10 um), Supercritical CO₂/EtOH+0.1% NH₄OH=45/55; 60 mL/min) to give 4-(2-cyclopentyl-2,8-diazaspiro[4.5]decan-8-yl)-2-(1,3-dimethyl-1H-pyrazol-4-yl)pyrido[3,4-d]pyrimidine (3.9 mg, second peak) as a yellow solid. ¹H NMR (400 MHz, DMSO-d₆) δ 9.04 (s, 1H), 8.43 (d, J=6.0 Hz, 1H), 8.32 (s, 1H), 7.75 (d, J=5.6 Hz, 1H), 3.93-3.84 (m, 2H), 3.83-3.81 (m, 3H), 3.77-3.71 (m, 2H), 2.58-2.54 (m, 5H), 2.44 (s, 2H), 2.42-2.35 (m, 1H), 1.75-1.62 (m, 10H), 1.53-1.44 (m, 2H), 1.43-1.33 (m, 2H). LCMS (ESI) m/z: 432.2 [M+H]⁺.

### Example 209

### 4-(2-methyl-2,8-diazaspiro[4.5]decan-8-yl)-2-(pyridin-4-yl)-6-(trifluoromethyl)pyrido[3,4-d]pyrimidine (Compound 209)

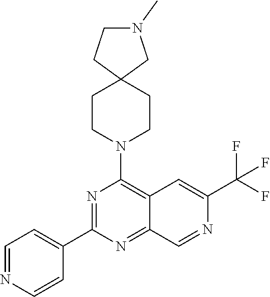 [SMILES: CN1CCC2(CCN(c3nc(-c4ccncc4)nc4cnc(C(F)(F)F)cc34)CC2)C1]

### Step 1: 5-chloro-N-(imino(pyridin-4-yl)methyl)-2-(trifluoromethyl)isonicotinamide

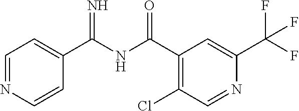 [SMILES: N=C(NC(=O)c1cc(C(F)(F)F)ncc1Cl)c1ccncc1]

To a solution of 5-chloro-2-(trifluoromethyl)isonicotinic acid (400 mg, 1.77 mmol) in DMF (11 mL) was added HATU (1.01 g, 2.66 mmol) and N,N-diisopropylethylamine (0.93 mL, 5.32 mmol) at room temperature. After stirring for 5 min, isonicotinimidamide hydrochloride (335 mg, 2.13 mmol) was added to this reaction mixture. The resulting mixture was stirred at room temperature for 5 h. The reaction mixture was added dropwise to water (30 mL) and stirred for 10 min. A white precipitate was formed and filtered off, the filter cake was washed with water (10 mL×2), petroleum ether (10 mL×2) and dried in vacuo to give the title compound (160 mg, 27%) as a yellow solid. LCMS (ESI) m/z: 328.9 [M+H]⁺.

### Step 2: 2-(pyridin-4-yl)-6-(trifluoromethyl)pyrido[3,4-d]pyrimidin-4-ol

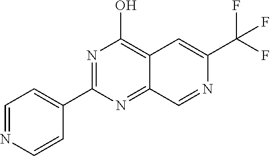 [SMILES: Oc1nc(-c2ccncc2)nc2cnc(C(F)(F)F)cc12]

To a solution of 5-chloro-N-(imino(pyridin-4-yl)methyl)-2-(trifluoromethyl)isonicotin amide (160 mg, 0.48 mmol) in DMF (5 mL) was added Cs₂CO₃ (476 mg, 1.45 mmol). The mixture was stirred at 100° C. for 3 h. After cooling to room temperature, water (20 mL) was added and the resulting mixture was stirred for 10 min. The mixture was adjusted to pH 5 with AcOH, then stirred for 10 min. A white precipitate was formed and filtered off, the filter cake was washed with water (10 mL×2), petroleum ether (10 mL×2) and dried in vacuo to give the title compound (190 mg, 92%) as a white solid. ¹H NMR (400 MHz, DMSO-d₆) δ 8.97 (s, 1H), 8.69-8.64 (m, 2H), 8.30-8.24 (m, 2H), 8.13 (s, 1H). LCMS (ESI) m/z: 292.9 [M+H]⁺.

### Step 3: tert-butyl 8-(2-(pyridin-4-yl)-6-(trifluoromethyl)pyrido[3,4-d]pyrimidin-4-yl)-2,8-diazaspiro[4.5]decane-2-carboxylate

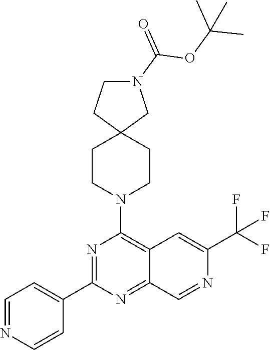 [SMILES: CC(C)(C)OC(=O)N1CCC2(CCN(c3nc(-c4ccncc4)nc4cnc(C(F)(F)F)cc34)CC2)C1]

To a solution of 2-(pyridin-4-yl)-6-(trifluoromethyl)pyrido[3,4-d]pyrimidin-4-ol (100 mg, 0.34 mmol) in acetonitrile (4 mL) was added DBU (156 mg, 1.03 mmol), tert-butyl 2,8-diazaspiro[4.5]decane-2-carboxylate (148 mg, 0.62 mmol) and BOP (272 mg, 0.62 mmol). The mixture was stirred at room temperature for 16 h. The reaction was quenched with water (30 mL), extracted with EtOAc (40 mL×2). The combined organic layers were washed with brine (30 mL), dried over anhydrous Na₂S04 and concentrated in vacuo. The crude residue was purified by flash chromatography (solvent gradient: 0-80% EtOAc in petroleum ether) to give the title compound (80 mg, 45%) as a yellow solid. LCMS (ESI) m/z: 515.0 [M+H]⁺.

### Step 4: 4-(2-methyl-2,8-diazaspiro[4.5]decan-8-yl)-2-(pyridin-4-yl)-6-(trifluoromethyl)pyrido[3,4-d]pyrimidine

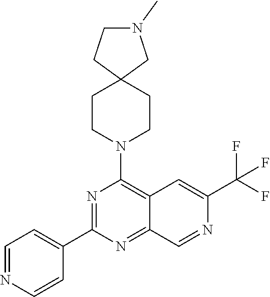 [SMILES: CN1CCC2(CCN(c3nc(-c4ccncc4)nc4cnc(C(F)(F)F)cc34)CC2)C1]

Following the procedure described in Example 203, step 3-4 and making non-critical variations as required to replace tert-butyl 8-(8-methoxy-2-(pyridin-4-yl)pyrido[3,4-d]pyrimidin-4-yl)-2,8-diazaspiro[4.5]decane-2-carboxylate with tert-butyl 8-(2-(pyridin-4-yl)-6-(trifluoromethyl)pyrido[3,4-d]pyrimidin-4-yl)-2,8-diazaspiro[4.5]decane-2-carboxylate, the title compound was obtained (20 mg, 24%) as a white solid. ¹H NMR (400 MHz, MeOD) δ 9.35 (s, 1H), 8.76-8.70 (m, 2H), 8.52-8.45 (m, 2H), 8.24 (s, 1H), 4.23-4.13 (m, 2H), 4.04 (ddd, J=13.4, 7.7, 4.0 Hz, 2H), 3.43 (t, J=7.5 Hz, 2H), 3.27 (s, 2H), 2.91 (s, 3H), 2.15 (t, J=7.5 Hz, 2H), 2.03-1.87 (m, 4H). LCMS (ESI) m/z: 429.1 [M+H]⁺.

### Example 210

### N,N-dimethyl-2-(8-(2-(3-methyl-1H-pyrazol-4-yl)pyrido[3,4-d]pyrimidin-4-yl)-2,8-diazaspiro[4.5]decan-2-yl)ethanesulfonamide (Compound 210)

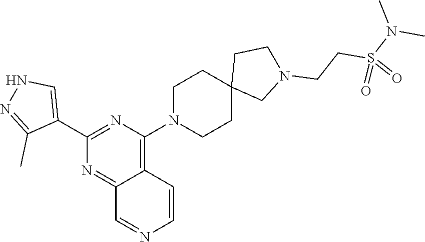 [SMILES: Cc1n[nH]cc1-c1nc(N2CCC3(CCN(CCS(=O)(=O)N(C)C)C3)CC2)c2ccncc2n1]

Following the procedure described in Example 109 and making non-critical variations as required to replace 4-(2,8-diazaspiro[4.5]decan-8-yl)-2-(4-pyridyl)pyrido[3,4-d]pyrimidine hydrochloride and ethenesulfonamide with 2-(3-methyl-1H-pyrazol-4-yl)-4-(2,8-diazaspiro[4.5]decan-8-yl)pyrido[3,4-d]pyrimidine hydrochloride and N,N-dimethylethenesulfonamide, the title compound was obtained (26 mg, 17%) as a yellow solid. ¹H NMR (400 MHz, MeOD) δ 9.08 (s, 1H), 8.41 (d, J=5.7 Hz, 1H), 8.20 (br s, 1H), 7.82 (d, J=5.8 Hz, 1H), 3.96 (ddd, J=12.0, 6.8, 4.0 Hz, 2H), 3.88 (ddd, J=12.0, 7.4, 3.9 Hz, 2H), 3.28-3.20 (m, 2H), 2.95-2.89 (m, 2H), 2.88 (s, 6H), 2.78-2.68 (m, 5H), 2.63 (s, 2H), 1.90-1.78 (m, 6H). Exchangeable amine NH proton not observed. LCMS (ESI) m/z: 485.2 [M+H]⁺.

### Example 211

### 3-((8-(2-(Pyridin-4-yl)pyrido[3,4-d]pyrimidin-4-yl)-2,8-diazaspiro[4.5]decan-2-yl)methyl)oxetan-3-ol (Compound 211) formate

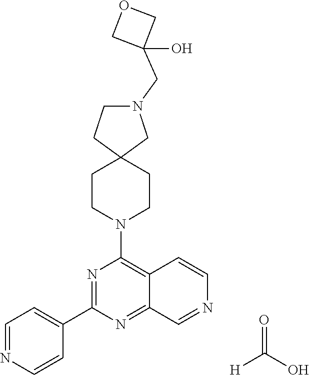 [SMILES: OC1(CN2CCC3(CCN(c4nc(-c5ccncc5)nc5cnccc45)CC3)C2)COC1 ; O=CO]

A solution of 4-(2,8-diazaspiro[4.5]decan-8-yl)-2-(4-pyridyl)pyrido[3,4-d]pyrimidine; hexahydrochloride (200 mg, 0.35 mmol), 1,5-dioxaspiro[2.3]hexane (46 mg, 0.53 mmol), MeOH (6.9 mL) and triethylamine (0.39 mL, 2.83 mmol) was stirred at 30° C. After 2 days, the solvent was removed under reduced pressure. The crude was purified by reverse phase column chromatography over C18 (MeCN/10 mM ammonium bicarbonate pH 10.0 aqueous buffer then 10 mM ammonium formate pH 3.8 aqueous buffer) to afford 3-[[8-[2-(4-pyridyl)pyrido[3,4-d]pyrimidin-4-yl]-2,8-diazaspiro[4.5]decan-2-yl]methyl]oxetan-3-ol, formate salt (71 mg, 0.16 mmol, 46% yield) as a brown solid. ¹H NMR (400 MHz, DMSO-d₆+D₂O) 9.12-9.10 (m, 1H), 8.65 (d, J=5.6 Hz, 2H), 8.51-8.43 (m, 1H), 8.30 (s, 1H), 8.21 (t, J=5.9 Hz, 2H), 7.79-7.75 (m, 1H), 4.46-4.42 (m, 4H), 3.91-3.80 (m, 2H), 3.77-3.72 (m, 2H), 3.09 (d, J=10.9 Hz, 2H), 2.98 (t, J=6.8 Hz, 2H), 2.83 (s, 2H), 1.82-1.64 (m, 6H). LCMS (ESI) m/z: 433.3 [M+H]⁺

### Example 212

### (2-methyl-8-(2-(pyridin-4-yl)pyrido[3,4-d]pyrimidin-4-yl)-2,8-diazaspiro[4.5]decan-3-yl)methanol (Compound 212)

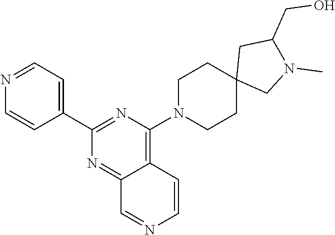 [SMILES: CN1CC2(CCN(c3nc(-c4ccncc4)nc4cnccc34)CC2)CC1CO]

Following the procedure described in Example 206, Step 2-4 and making non-critical variations as required to replace cyclopentanone with formaldehyde, the title compound was obtained as a mixture of enantiomers (12 mg, 15%) as a yellow solid. ¹H NMR (400 MHz, DMSO-d₆) δ 9.25 (s, 1H), 8.77 (d, J=6.0 Hz, 2H), 8.58 (d, J=5.6 Hz, 1H), 8.32 (d, J=6.0 Hz, 2H), 7.89 (d, J=5.6 Hz, 1H), 4.46 (t, J=5.2 Hz, 1H), 4.08-3.92 (m, 2H), 3.91-3.82 (m, 2H), 3.48 (m, 1H), 3.00-2.96 (m, 1H), 2.45-2.29 (m, 2H), 2.26 (s, 3H), 2.09-2.05 (m, 1H), 1.83-1.53 (m, 6H). LCMS (ESI) m/z: 391.1[M+H]⁺.

### Example 213

### 2-(5-(difluoromethyl)-1H-pyrazol-4-yl)-4-(2,8-diazaspiro[4.5]decan-8-yl)pyrido[3,4-d]pyrimidine (Compound 213) trifluoroacetate

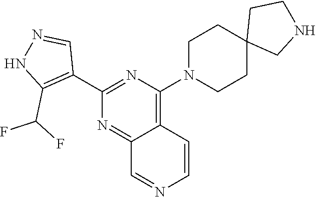 [SMILES: FC(F)c1[nH]ncc1-c1nc(N2CCC3(CCNC3)CC2)c2ccncc2n1]

Following the procedure described in Example 153, Step 2 and making non-critical variations as required to replace 3-chloro-4-(4,4,5,5-tetramethyl-1,3,2-dioxaborolan-2-yl)-1H-pyrazole with 5-(difluoromethyl)-4-(4,4,5,5-tetramethyl-1,3,2-dioxaborolan-2-yl)-1H-pyrazole (prepared according to the procedure in Bioorg. Med. Chem. Lett., 2016, 26, 534), the title compound was obtained (6 mg, 8%) as a white solid. ¹H NMR (400 MHz, DMSO-d₆) δ 13.70 (s, 1H), 9.16 (s, 1H), 8.93-8.84 (m, 2H), 8.63-8.46 (m, 2H), 7.95-7.62 (m, 2H), 4.09-3.78 (m, 4H), 3.35-3.26 (m, 2H), 3.18-3.04 (m, 2H), 1.93 (t, J=7.2 Hz, 2H), 1.85-1.74 (m, 4H). LCMS (ESI) m/z: 386.0 [M+H]⁺.

### Example 214

### N-methyl-4-(2-methyl-2,8-diazaspiro[4.5]decan-8-yl)-2-(pyridin-4-yl)pyrido[3,4-d]pyrimidin-6-amine (Compound 214)

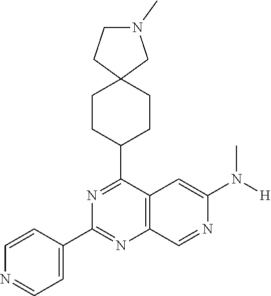 [SMILES: CNc1cc2c(C3CCC4(CC3)CCN(C)C4)nc(-c3ccncc3)nc2cn1]

### Step 1: 6-chloro-4-(2-methyl-2,8-diazaspiro[4.5]decan-8-yl)-2-(pyridin-4-yl)pyrido[3,4-d]pyrimidine

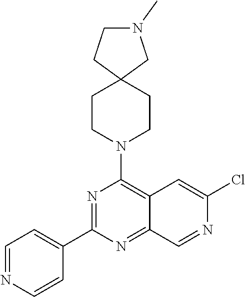 [SMILES: CN1CCC2(CCN(c3nc(-c4ccncc4)nc4cnc(Cl)cc34)CC2)C1]

Following the procedure described in Example 203, step 3-4 and making non-critical variations as required to replace tert-butyl 8-(8-methoxy-2-(pyridin-4-yl)pyrido[3,4-d]pyrimidin-4-yl)-2,8-diazaspiro[4.5]decane-2-carboxylate with tert-butyl 8-(6-chloro-2-(pyridin-4-yl)pyrido[3,4-d]pyrimidin-4-yl)-2,8-diazaspiro[4.5]decane-2-carboxylate, the title compound was obtained (310 mg, 75%) as a yellow solid. LCMS (ESI) m/z: 395.2 [M+H]⁺.

### Step 2: tert-butyl methyl(4-(2-methyl-2,8-diazaspiro[4.5]decan-8-yl)-2-(pyridin-4-yl)pyrido[3,4-d]pyrimidin-6-yl)carbamate

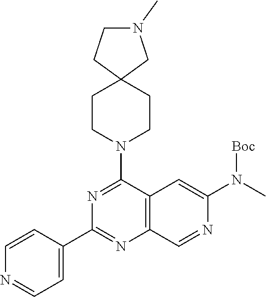 [SMILES: CN1CCC2(CCN(c3nc(-c4ccncc4)nc4cnc(N(C)C(=O)OC(C)(C)C)cc34)CC2)C1]

To a solution of 6-chloro-4-(2-methyl-2,8-diazaspiro[4.5]decan-8-yl)-2-(4-pyridyl)pyrido[3,4-d]pyrimidine (200 mg, 0.51 mmol) and tert-butyl N-methylcarbamate (264 mg, 2.01 mmol) in dioxane (3 mL) was added 2-(dicyclohexylphosphino)-2′,4′,6′-triisopropylbiphenyl (100 mg, 0.21 mmol), tris(dibenzylideneacetone)dipalladium (100 mg, 0.11 mmol) and sodium tert-butoxide (100 mg, 1.04 mmol). The mixture was stirred at 130° C. for 2 h under microwave. After colling to room temperature, the reaction mixture was concentrated in vacuo. The residue was purified by prep-TLC (DCM/MeOH=10:1) to give the title compound (60 mg, 24%) as a yellow solid. LCMS (ESI) m/z: 490.2 [M+H]⁺.

### Step 3: N-methyl-4-(2-methyl-2,8-diazaspiro[4.5]decan-8-yl)-2-(pyridin-4-yl)pyrido[3,4-d]pyrimidin-6-amine

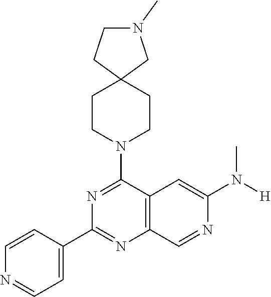 [SMILES: CNc1cc2c(N3CCC4(CCN(C)C4)CC3)nc(-c3ccncc3)nc2cn1]

To a solution of tert-butyl methyl(4-(2-methyl-2,8-diazaspiro[4.5]decan-8-yl)-2-(pyridin-4-yl)pyrido[3,4-d]pyrimidin-6-yl)carbamate (60 mg, 0.12 mmol) in DCM (1 mL) was added trifluoroacetic acid (0.24 mL, 3.16 mmol). The mixture was stirred at room temperature for 3 h and then concentrated in vacuo. The crude residue was purified by reverse phase chromatography (acetonitrile 27-57%/0.05% NH₃·H₂O+10 mM NH₄HCO₃ in water) to give the title compound (14 mg, 27%) as a yellow solid. ¹H NMR (400 MHz, DMSO-d₆) δ 8.88 (s, 1H), 8.69 (d, J=4.8 Hz, 2H), 8.24 (d, J=4.8 Hz, 2H), 7.03-6.95 (m, 1H), 6.58 (s, 1H), 3.84-3.77 (m, 2H), 3.75-3.68 (m, 2H), 2.86 (d, J=4.8 Hz, 3H), 2.74-2.64 (m, 2H), 2.60-2.54 (m, 2H), 2.36 (s, 3H), 1.83-1.77 (m, 2H), 1.76-1.70 (m, 4H). LCMS (ESI) m/z: 390.2 [M+H]⁺.

### Example 215

### N,N-dimethyl-2-(8-(2-(pyridin-4-yl)pyrido[3,4-d]pyrimidin-4-yl)-2,8-diazaspiro[4.5]decan-2-yl)ethanesulfonamide (Compound 215)

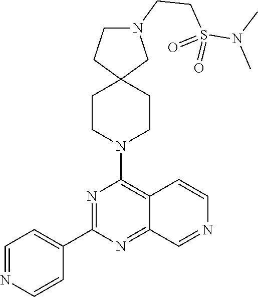 [SMILES: CN(C)S(=O)(=O)CCN1CCC2(CCN(c3nc(-c4ccncc4)nc4cnccc34)CC2)C1]

Following the procedure described in Example 109 and making non-critical variations as required to replace ethenesulfonamide with N,N-dimethylethenesulfonamide, the title compound was obtained as a white solid. ¹H NMR (400 MHz, DMSO-d₆) δ 9.26 (s, 1H), 8.80-8.74 (m, 2H), 8.59 (d, J=5.6 Hz, 1H), 8.34-8.30 (m, 2H), 7.89 (d, J=5.6 Hz, 1H), 4.03-3.86 (m, 4H), 3.22 (t, J=7.2 Hz, 2H), 2.78 (s, 6H), 2.78-2.73 (m, 2H), 2.62 (t, J=6.8 Hz, 2H), 2.51 (s, 2H), 1.82-1.68 (m, 6H). LCMS (ESI) m/z: 482.1 [M+H]⁺.

### Example 216

### 4-(3-(fluoromethyl)-2,8-diazaspiro[4.5]decan-8-yl)-2-(pyridin-4-yl)pyrido[3,4-d]pyrimidine (Compound 216)

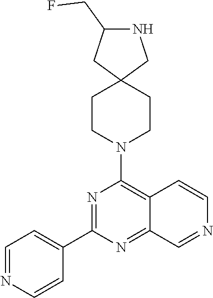 [SMILES: FCC1CC2(CCN(c3nc(-c4ccncc4)nc4cnccc34)CC2)CN1]

### Step 1: 2-benzyl 8-tert-butyl 3-(hydroxymethyl)-2,8-diazaspiro[4.5]decane-2,8-dicarboxylate

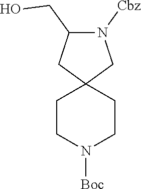 [SMILES: CC(C)(C)OC(=O)N1CCC2(CC1)CC(CO)N(C(=O)OCc1ccccc1)C2]

To a solution of 2-benzyl 8-tert-butyl 3-ethyl 2,8-diazaspiro[4.5]decane-2,3,8-tricarboxylate (5.5 g, 12.32 mmol) (prepared according to the procedure in WO201887602) in THF (30 mL) was added NaBH₄ (1.4 g, 36.95 mmol). Then LiCl (1.6 g, 36.95 mmol) was added slowly to the mixture. The reaction was stirred at room temperature for 16 h. The reaction was quenched with sat. aq. NH₄Cl (30 mL) and extracted with EtOAc (80 mL×2). The combined organic layers were dried over anhydrous Na₂SO₄, filtered and concentrated in vacuo. The crude residue was purified by silica gel chromatography (solvent gradient: 0-30% EtOAc in petroleum ether) to give the title compound (3.1 g, 60%) as yellow oil. LCMS (ESI) m/z: 405.1 [M+H]⁺.

### Step 2: 2-benzyl 8-tert-butyl 3-(fluoromethyl)-2,8-diazaspiro[4.5]decane-2,8-dicarboxylate

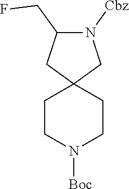 [SMILES: CC(C)(C)OC(=O)N1CCC2(CC1)CC(CF)N(C(=O)OCc1ccccc1)C2]

To a solution of perfluorobutanesulfonyl fluoride (291 mg, 0.96 mmol) and 2-tert-butyl-1,1,3,3-tetramethylguanidine (228 mg, 1.33 mmol) in THF (8 mL) was added 2-benzyl 8-tert-butyl 3-(hydroxymethyl)-2,8-diazaspiro[4.5]decane-2,8-dicarboxylate (300 mg, 0.74 mmol). The mixture was stirred at room temperature for 2 h. The reaction mixture was diluted with water (30 mL), extracted with EtOAc (20 mL×2). The combined organic layers were dried over anhydrous Na₂SO₄, filtered and concentrated in vacuo. The crude residue was purified by silica gel chromatography (solvent gradient: 0-20% EtOAc in petroleum ether) to give the title compound (0.15 g, 50%) as a white solid. LCMS (ESI) m/z: 429.1 [M+23]⁺.

### Step 3: benzyl 3-(fluoromethyl)-8-(2-(pyridin-4-yl)pyrido[3,4-d]pyrimidin-4-yl)-2,8-diazaspiro[4.5]decane-2-carboxylate

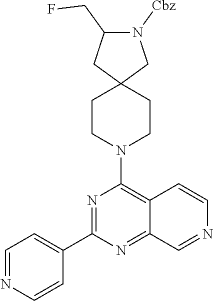 [SMILES: O=C(OCc1ccccc1)N1CC2(CCN(c3nc(-c4ccncc4)nc4cnccc34)CC2)CC1CF]

Following the procedure described in Example 206, step 3-4 and making non-critical variations as required to replace tert-butyl 2-cyclopentyl-3-(hydroxymethyl)-2,8-diazaspiro[4.5]decane-8-carboxylate with 2-benzyl 8-tert-butyl 3-(fluoromethyl)-2,8-diazaspiro[4.5]decane-2,8-dicarboxylate, the title compound was obtained (110 mg, 55%) as a yellow solid. LCMS (ESI) m/z: 513.1 [M+H]⁺.

### Step 4: 4-(3-(fluoromethyl)-2,8-diazaspiro[4.5]decan-8-yl)-2-(pyridin-4-yl)pyrido[3,4-d]pyrimidine

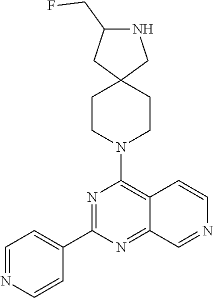 [SMILES: FCC1CC2(CCN(c3nc(-c4ccncc4)nc4cnccc34)CC2)CN1]

To a solution of benzyl 3-(fluoromethyl)-8-(2-(pyridin-4-yl)pyrido[3,4-d]pyrimidin-4-yl)-2,8-diazaspiro[4.5]decane-2-carboxylate (110 mg, 0.21 mmol) in EtOAc (5 mL) was added 10% palladium on carbon (50 mg) at room temperature. After the addition, the reaction mixture was stirred at room temperature for 1 h under hydrogen atmosphere. The mixture was concentrated in vacuo. The crude residue was purified by reverse phase chromatography (acetonitrile 35-65%/0.2% formic acid in water) to give the title compound (16 mg, 17%, mixture of enantiomers) as a white solid. ¹H NMR (400 MHz, DMSO-d₆) δ 9.25 (s, 1H), 8.81-8.72 (m, 2H), 8.58 (d, J=5.6 Hz, 1H), 8.34-8.30 (m, 2H), 7.89 (d, J=5.6 Hz, 1H), 4.40-4.26 (m, 1H), 4.24-4.15 (m, 1H), 3.97-3.91 (m, 4H), 3.54-3.46 (m, 1H), 2.85-2.78 (m, 1H), 2.70-2.65 (m, 1H), 1.91-1.80 (m, 1H), 1.77-1.63 (m, 4H), 1.29-1.22 (m, 1H). LCMS (ESI) m/z: 379.0 [M+H]⁺.

### Example 217

### 4-(3-(methoxymethyl)-2-methyl-2,8-diazaspiro[4.5]decan-8-yl)-2-(pyridin-4-yl)pyrido[3,4-d]pyrimidine (Compound 217)

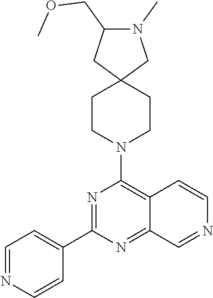 [SMILES: COCC1CC2(CCN(c3nc(-c4ccncc4)nc4cnccc34)CC2)CN1C]

### Step 1: 2-benzyl 8-tert-butyl 3-(methoxymethyl)-2,8-diazaspiro[4.5]decane-2,8-dicarboxylate

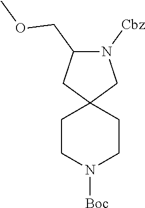 [SMILES: COCC1CC2(CCN(C(=O)OC(C)(C)C)CC2)CN1C(=O)OCc1ccccc1]

To a solution of 2-benzyl 8-tert-butyl 3-(hydroxymethyl)-2,8-diazaspiro[4.5]decane-2,8-dicarboxylate (400 mg, 0.99 mmol) and silver(II) oxide (250 mg, 1.98 mmol) in MeCN (5 mL) was added iodomethane (0.31 mL, 4.94 mmol). Then the reaction mixture was stirred at 60° C. for 24 h. After cooling to room temperature, the reaction mixture was diluted with water (10 mL), extracted with EtOAc (30 mL×2). The combined organic layers were dried over anhydrous Na₂SO₄, filtered and concentrated in vacuo. The crude residue was purified by silica gel chromatography (solvent gradient: 0-30% EtOAc in petroleum ether) to give the title compound (260 mg, 63%) as colorless oil. LCMS (ESI) m/z: 419.5 [M+H]⁺.

### Step 2: benzyl 3-(methoxymethyl)-8-(2-(pyridin-4-yl)pyrido[3,4-d]pyrimidin-4-yl)-2,8-diazaspiro[4.5]decane-2-carboxylate

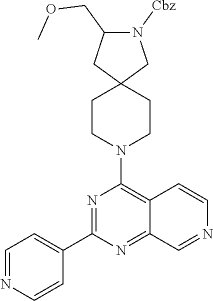 [SMILES: COCC1CC2(CCN(c3nc(-c4ccncc4)nc4cnccc34)CC2)CN1C(=O)OCc1ccccc1]

Following the procedure described in Example 206, step 3-4 and making non-critical variations as required to replace tert-butyl 2-cyclopentyl-3-(hydroxymethyl)-2,8-diazaspiro[4.5]decane-8-carboxylate with 2-benzyl 8-tert-butyl 3-(methoxymethyl)-2,8-diazaspiro[4.5]decane-2,8-dicarboxylate, the title compound was obtained (250 mg, 86%) as a yellow solid. LCMS (ESI) m/z: 525.4 [M+H]⁺.

### Step 3: 4-(3-(methoxymethyl)-2,8-diazaspiro[4.5]decan-8-yl)-2-(pyridin-4-yl)pyrido[3,4-d]pyrimidine

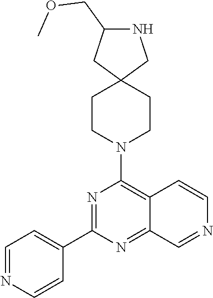 [SMILES: COCC1CC2(CCN(c3nc(-c4ccncc4)nc4cnccc34)CC2)CN1]

Following the procedure described in Example 216, step 4 and making non-critical variations as required to replace benzyl 3-(fluoromethyl)-8-(2-(pyridin-4-yl)pyrido[3,4-d]pyrimidin-4-yl)-2,8-diazaspiro[4.5]decane-2-carboxylate with benzyl 3-(methoxymethyl)-8

(2-(pyridin-4-yl)pyrido[3,4-d]pyrimidin-4-yl)-2,8-diazaspiro[4.5]decane-2-carboxylate, the title compound was obtained (160 mg, 92%) as yellow oil. LCMS (ESI) m/z: 391.1 [M+H]⁺.

### Step 4: 4-(3-(methoxymethyl)-2-methyl-2,8-diazaspiro[4.5]decan-8-yl)-2-(pyridin-4-yl)pyrido[3,4-d]pyrimidine

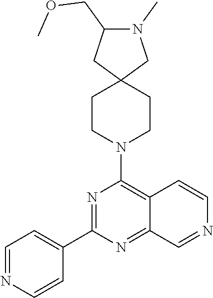 [SMILES: COCC1CC2(CCN(c3nc(-c4ccncc4)nc4cnccc34)CC2)CN1C]

Following the procedure described in Example 102 and making non-critical variations as required to replace 4-(2,8-diazaspiro[4.5]decan-8-yl)-2-(4-pyridyl)pyrido[3,4-d]pyrimidine hydrochloride with 4-(3-(methoxymethyl)-2,8-diazaspiro[4.5]decan-8-yl)-2-(pyridin-4-yl)pyrido[3,4-d]pyrimidine, the title compound was obtained (10 mg, 11%, mixture of enantiomers) as a white solid. ¹H NMR (400 MHz, CD₃OD) δ 9.26 (s, 1H), 8.71 (d, J=6.0 Hz, 2H), 8.55 (d, J=6.0 Hz, 1H), 8.46 (d, J=6.0 Hz, 2H), 7.93 (d, J=5.6 Hz, 1H), 4.86-4.83 (m, 1H), 4.63-4.58 (m, 2H), 4.17-4.05 (m, 2H), 3.99-3.88 (m, 2H), 3.59-3.55 (m, 2H), 3.42 (s, 3H), 2.64 (s, 3H), 2.15-2.09 (m, 1H), 1.95-1.79 (m, 5H). LCMS (ESI) m/z: 405.1 [M+H]⁺.

### Example 218

### 1-(2-(pyridin-4-yl)pyrido[3,4-d]pyrimidin-4-yl)hexahydrospiro[piperidine-4,7′-pyrrolo[2,1-c][1,4]oxazine] (Compound 218)

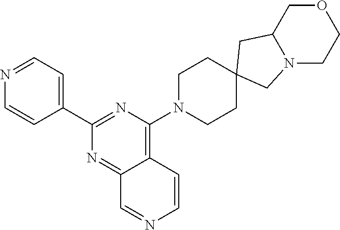 [SMILES: c1cc(-c2nc(N3CCC4(CC3)CC3COCCN3C4)c3ccncc3n2)ccn1]

### Step 1: tert-butyl 2-(2-chloroacetyl)-3-(hydroxymethyl)-2,8-diazaspiro[4.5]decane-8-carboxylate

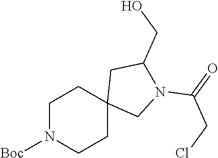 [SMILES: CC(C)(C)OC(=O)N1CCC2(CC1)CC(CO)N(C(=O)CCl)C2]

To a solution of tert-butyl 3-(hydroxymethyl)-2,8-diazaspiro[4.5]decane-8-carboxylate (70 mg, 0.26 mmol) in DCM (3 mL) was added triethylamine (0.11 mL, 0.78 mmol) and chloroacetyl chloride (0.02 mL, 0.28 mmol). The reaction was stirred at 0° C. for 3 h. The reaction was quenched with water (15 mL) and extracted with DCM (20 mL×2). The combined organic layers were washed with brine (15 mL), dried over anhydrous Na₂SO₄, filtered and concentrated in vacuo to give the title compound (80 mg, 89%) as yellow oil. LCMS (ESI) m/z: 347.1 [M+H]⁺.

### Step 2: tert-butyl 4′-oxohexahydrospiro[piperidine-4,7′-pyrrolo[2,1-c] [1,4]oxazine]-1-carboxylate

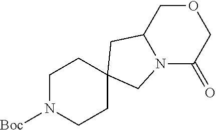 [SMILES: CC(C)(C)OC(=O)N1CCC2(CC1)CC1COCC(=O)N1C2]

To a solution of sodium hydride (14 mg, 0.35 mmol, 60%) in THF (3 mL) was added tert-butyl 2-(2-chloroacetyl)-3-(hydroxymethyl)-2,8-diazaspiro[4.5]decane-8-carboxylate (80 mg, 0.23 mmol) at 0° C. The reaction was stirred at 0° C. for 16 h. The reaction was quenched with sat. aq. NH₄Clsolution (20 mL) and extracted with ethyl acetate (20 mL×2). The combined organic layers were washed with brine (15 mL), dried over anhydrous Na₂SO₄, filtered and concentrated in vacuo to give the title compound (70 mg, 98%) as yellow oil. LCMS (ESI) m/z: 333.0 [M+Na]⁺.

### Step 3: tert-butyl hexahydrospiro[piperidine-4,7′-pyrrolo[2,1-c][1,4]oxazine]-1-carboxylate

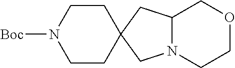 [SMILES: CC(C)(C)OC(=O)N1CCC2(CC1)CC1COCCN1C2]

To a solution of tert-butyl 4′-oxohexahydrospiro[piperidine-4,7′-pyrrolo[2,1-c][1,4]oxazine]-1-carboxylate (70 mg, 0.23 mmol) in THF (2 mL) was added borane dimethyl sulfide complex (0.11 mL, 1.13 mmol). The mixture was stirred at room temperature for 16 h. The reaction was quenched with MeOH (4 mL), and then aq. HCl (1 mL, 1N) was added. The solution was stirred at 50° C. for 3 h. After cooling to room temperature, the reaction was concentrated in vacuo to give the title compound (60 mg, 90%) as yellow oil. LCMS (ESI) m/z: 196.9 [M-100+H]⁺

### Step 4: 1-(2-(pyridin-4-yl)pyrido[3,4-d]pyrimidin-4-yl)hexahydrospiro[piperidine-4,7′-pyrrolo[2,1-c] [1,4]oxazine]

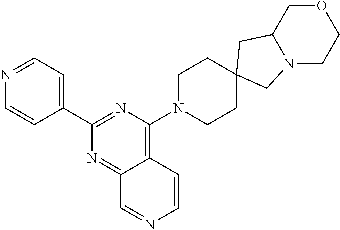 [SMILES: c1cc(-c2nc(N3CCC4(CC3)CC3COCCN3C4)c3ccncc3n2)ccn1]

Following the procedure described in Example 206, step 3-4 and making non-critical variations as required to replace tert-butyl 2-cyclopentyl-3-(hydroxymethyl)-2,8-diazaspiro[4.5]decane-8-carboxylate with tert-butyl hexahydrospiro[piperidine-4,7′-pyrrolo[2,1-c][1,4]oxazine]-1-carboxylate, the title compound (20 mg, 22%, as a mixture of enantiomers) was obtained as a brown solid. ¹H NMR (400 MHz, DMSO-d₆) δ 9.26 (s, 1H), 8.82-8.72 (m, 2H), 8.59 (d, J=5.2 Hz, 1H), 8.35-8.28 (m, 2H), 7.89 (d, J=6.0 Hz, 1H), 4.06-3.99 (m, 1H), 3.96-3.83 (m, 4H), 3.76-3.70 (m, 1H), 3.43-3.41 (m, 1H), 3.16-3.12 (m, 1H), 3.08-3.02 (m, 1H), 2.85-2.81 (m, 1H), 2.22-2.12 (m, 2H), 2.03-1.99 (m, 1H), 1.79-1.70 (m, 4H), 1.31-1.12 (m, 2H). LCMS (ESI) m/z: 403.1 [M+H]⁺.

### Example 219

### 4-(2-(2-fluoroethyl)-2,8-diazaspiro[4.5]decan-8-yl)-2-(3-methyl-1H-pyrazol-4-yl)pyrido[3,4-d]pyrimidine (Compound 219)

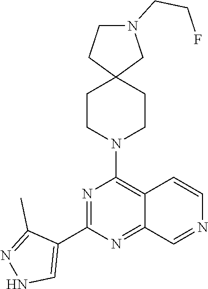 [SMILES: Cc1n[nH]cc1-c1nc(N2CCC3(CCN(CCF)C3)CC2)c2ccncc2n1]

Following the procedure described in Example 107, Step 1 and making non-critical variations as required to replace 4-(2,8-diazaspiro[4.5]decan-8-yl)-2-(4-pyridyl)pyrido[3,4-d]pyrimidine hydrochloride and methyl 2-bromo-2-methylpropanoate with 2-(3-methyl-1H-pyrazol-4-yl)-4-(2,8-diazaspiro[4.5]decan-8-yl)pyrido[3,4-d]pyrimidine hydrochloride and 1-bromo-2-fluoroethane, the title compound was obtained as a white solid. ¹H NMR (400 MHz, DMSO-d₆) δ 12.84 (s, 1H), 9.08 (s, 1H), 8.44 (d, J=5.6 Hz, 1H), 8.15 (s, 1H), 7.75 (d, J=5.6 Hz, 1H), 4.60-4.44 (m, 2H), 3.86-3.74 (m, 4H), 2.76-2.66 (m, 2H), 2.65 (s, 3H), 2.61 (t, J=6.8 Hz, 2H), 2.55-2.50 (m, 2H), 1.76-1.64 (m, 6H). LCMS (ESI) m/z: 396.2 [M+H]⁺

### Example 220

### 4-(2-(2,2-difluoroethyl)-2,8-diazaspiro[4.5]decan-8-yl)-2-(3-methyl-1H-pyrazol-4-yl)pyrido[3,4-d]pyrimidine Compound 220)

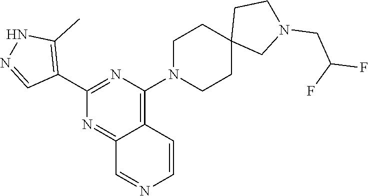 [SMILES: Cc1[nH]ncc1-c1nc(N2CCC3(CCN(CC(F)F)C3)CC2)c2ccncc2n1]

Following the procedure described in Example 107, Step 1 and making non-critical variations as required to replace 4-(2,8-diazaspiro[4.5]decan-8-yl)-2-(4-pyridyl)pyrido[3,4-d]pyrimidine hydrochloride and methyl 2-bromo-2-methylpropanoate with 2-(3-methyl-1H-pyrazol-4-yl)-4-(2,8-diazaspiro[4.5]decan-8-yl)pyrido[3,4-d]pyrimidine hydrochloride and 2,2-difluoroethyl trifluoromethanesulfonate, the title compound was obtained as a white solid. ¹H NMR (400 MHz, DMSO-d₆) δ 12.86 (s, 1H), 9.07 (s, 1H), 8.44 (d, J=5.6 Hz, 1H), 8.07 (s, 1H), 7.76 (d, J=5.6 Hz, 1H), 6.27-5.92 (m, 1H), 3.86-3.74 (m, 4H), 2.83 (m, 2H), 2.73-2.68 (m, 2H), 2.68-2.65 (m, 2H), 2.57 (s, 3H), 1.76-1.67 (m, 6H). LCMS (ESI) m/z: 414.2[M+H]⁺.

### Examples 221 & 222

### (R)-4-(3-(methoxymethyl)-2,8-diazaspiro[4.5]decan-8-yl)-2-(pyridin-4-yl)pyrido[3,4-d]pyrimidine (Compound 221) & (S)-4-(3-(methoxymethyl)-2,8-diazaspiro[4.5]decan-8-yl)-2-(pyridin-4-yl)pyrido[3,4-d]pyrimidine (Compound 222)

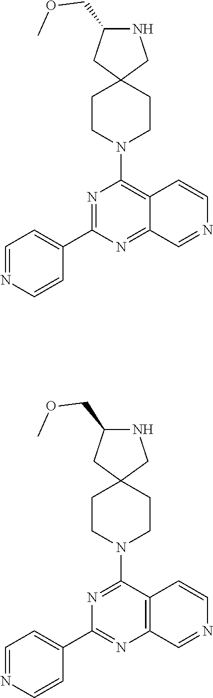 [SMILES: COC[C@H]1CC2(CCN(c3nc(-c4ccncc4)nc4cnccc34)CC2)CN1 ; COC[C@@H]1CC2(CCN(c3nc(-c4ccncc4)nc4cnccc34)CC2)CN1]

4-(3-(methoxymethyl)-2,8-diazaspiro[4.5]decan-8-yl)-2-(pyridin-4-yl)pyrido[3,4-d]pyrimidine (100 mg, 0.26 mmol) was separated by using chiral SFC (Chiralpak AD (250 mm*30 mm, 10 um), Supercritical CO₂/EtOH+0.1% NH₄OH=55/45; 60 mL/min) to give (R)-4-(3-(methoxymethyl)-2,8-diazaspiro[4.5]decan-8-yl)-2-(pyridin-4-yl)pyrido[3,4-d]pyrimidine (30 mg, first peak) and (S)-4-(3-(methoxymethyl)-2,8-diazaspiro[4.5]decan-8-yl)-2-(pyridin-4-yl)pyrido[3,4-d]pyrimidine (19 mg, second peak) both as white solid. Absolute configuration was arbitrarily assigned to each enantiomer. Example 221 (first peak): ¹H NMR (400 MHz, CD₃OD) δ 9.24 (s, 1H), 8.76 (d, J=6.0 Hz, 2H), 8.58 (d, J=6.0 Hz, 1H), 8.31 (d, J=6.0 Hz, 2H), 7.87 (d, J=5.6 Hz, 1H), 3.98-3.87 (m, 4H), 3.38-3.34 (m, 3H), 3.28 (s, 3H), 2.93-2.85 (m, 2H), 1.98-1.91 (m, 1H), 1.78-1.74 (m, 4H), 1.40-1.35 (m, 1H). LCMS (ESI) m/z: 391.1 [M+H]⁺. Example 222 (second peak): ¹H NMR (400 MHz, CD₃OD): δ 9.24 (s, 1H), 8.76 (d, J=6.0 Hz, 2H), 8.58 (d, J=6.0 Hz, 1H), 8.31 (d, J=6.0 Hz, 2H), 7.87 (d, J=5.6 Hz, 1H), 3.98-3.87 (m, 4H), 3.38-3.34 (m, 3H), 3.28 (s, 3H), 2.93-2.85 (m, 2H), 1.98-1.91 (m, 1H), 1.78-1.71 (m, 4H), 1.40-1.32 (m, 1H). LCMS (ESI) m/z: 391.1 [M+H]⁺.

### Example 223

### 4-(4,4-difluoro-2,8-diazaspiro[4.5]decan-8-yl)-2-(pyridin-4-yl)pyrido[3,4-d]pyrimidine (Compound 223) formate

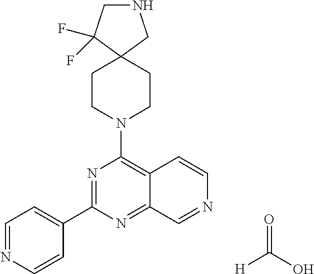 [SMILES: FC1(F)CNCC12CCN(c1nc(-c3ccncc3)nc3cnccc13)CC2 ; O=CO]

### Step 1: 8-benzyl 2-tert-butyl 4-oxo-2,8-diazaspiro[4.5]decane-2,8-dicarboxylate

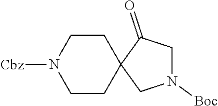 [SMILES: CC(C)(C)OC(=O)N1CC(=O)C2(CCN(C(=O)OCc3ccccc3)CC2)C1]

To a solution of 8-benzyl 2-tert-butyl 4-hydroxy-2,8-diazaspiro[4.5]decane-2,8-dicarboxylate (300 mg, 0.77 mmol) in DCM (6 mL) was added Dess-Martin periodinane (489 mg, 1.15 mmol) at 0° C. The mixture was stirred at room temperature for 16 h under nitrogen atmosphere. The reaction mixture was diluted with EtOAc (100 mL), washed with sat. aq. NaHCO₃ (50 mL×2) and brine (50 mL), dried over anhydrous Na₂SO₄, filtered, concentrated in vacuo. The residue was purified by silica gel chromatography (solvent gradient: 0-20% EtOAc in petroleum ether) to give the title compound (160 mg, 54%) as yellow oil. LCMS (ESI) m/z: 289.1 [M-100+H]⁺.

### Step 2: 8-benzyl 2-tert-butyl 4,4-difluoro-2,8-diazaspiro[4.5]decane-2,8-dicarboxylate

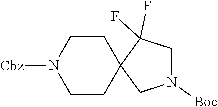 [SMILES: CC(C)(C)OC(=O)N1CC(F)(F)C2(CCN(C(=O)OCc3ccccc3)CC2)C1]

To a solution of 8-benzyl 2-tert-butyl 4-oxo-2,8-diazaspiro[4.5]decane-2,8-dicarboxylate (140 mg, 0.36 mmol) in 1,2-dichloroethane (2 mL) was added Deoxo-Fluor (166 uL, 0.90 mmol). The reaction mixture was heated to 60° C. for 16 h. After cooling to room temperature, the reaction mixture was quenched with sat. aq. NaHCO₃ (20 mL), extracted with DCM (50 mL). The organic layer was washed with brine (20 mL), dried over anhydrous Na₂SO₄, filtered and concentrated in vacuo. The residue was purified by prep-TLC (petroleum ether/EtOAc=5:1) to give the title compound (90 mg, 61%) as yellow oil. ¹H NMR (400 MHz, CDCl₃) δ 7.41-7.30 (m, 5H), 5.14 (s, 2H), 4.12-3.96 (m, 2H), 3.78-3.63 (m, 2H), 3.51-3.39 (m, 2H), 3.10-2.93 (m, 2H), 1.85-1.72 (m, 2H), 1.56-1.50 (m, 2H), 1.47 (s, 9H). LCMS (ESI) m/z: 311.2 [M-100+H]⁺.

### Step 3: tert-butyl 4,4-difluoro-2,8-diazaspiro[4.5]decane-2-carboxylate

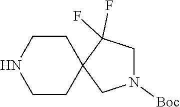 [SMILES: CC(C)(C)OC(=O)N1CC(F)(F)C2(CCNCC2)C1]

To a solution of 8-benzyl 2-tert-butyl 4,4-difluoro-2,8-diazaspiro[4.5]decane-2,8-dicarboxylate (110 mg, 0.28 mmol) in EtOAc (2 mL) was added 10% palladium on carbon (25 mg). The mixture was stirred at room temperature for 16 h under hydrogen atmosphere (15 psi). The mixture was filtered and the filtrate was concentrated in vacuo to give the title compound (60 mg, crude) as yellow oil that required no further purification. LCMS (ESI) m/z: 277.2 [M+H]⁺.

### Step 4: 4-(4,4-difluoro-2,8-diazaspiro[4.5]decan-8-yl)-2-(pyridin-4-yl)pyrido[3,4-d]pyrimidine formate

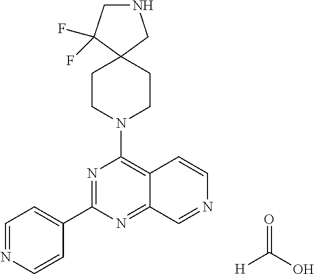 [SMILES: FC1(F)CNCC12CCN(c1nc(-c3ccncc3)nc3cnccc13)CC2 ; O=CO]

Following the procedure described in Example 101, step 3 and making non-critical variations as required to replace tert-butyl 2,8-diazaspiro[4.5]decane-2-carboxylate with tert-butyl 4,4-difluoro-2,8-diazaspiro[4.5]decane-2-carboxylate, the title compound (15 mg, 30%) was obtained as a yellow solid. ¹H NMR (400 MHz, DMSO-d₆) δ 9.29 (s, 1H), 8.81-8.74 (m, 2H), 8.61 (d, J=5.6 Hz, 1H), 8.38-8.31 (m, 2H), 8.14 (s, 1H), 7.94 (d, J=5.6 Hz, 1H), 4.49-4.35 (m, 2H), 3.65-3.54 (m, 2H), 3.52-3.43 (m, 2H), 3.30 (s, 2H), 2.06-1.91 (m, 2H), 1.86-1.76 (m, 2H). LCMS (ESI) m/z: 383.2 [M+H]⁺.

### Example 224

### N-((8-(2-(pyridin-4-yl)pyrido[3,4-d]pyrimidin-4-yl)-2,8-diazaspiro[4.5]decan-3-yl)methyl)methanesulfonamide (Compound 224) formate

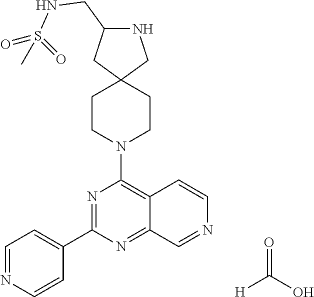 [SMILES: CS(=O)(=O)NCC1CC2(CCN(c3nc(-c4ccncc4)nc4cnccc34)CC2)CN1 ; O=CO]

### Step 1: 2-benzyl 8-tert-butyl 3-(((methylsulfonyl)oxy)methyl)-2,8-diazaspiro[4.5]decane-2,8-dicarboxylate

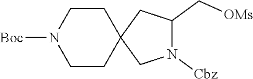 [SMILES: CC(C)(C)OC(=O)N1CCC2(CC1)CC(COS(C)(=O)=O)N(C(=O)OCc1ccccc1)C2]

To a solution of 2-benzyl 8-tert-butyl 3-(hydroxymethyl)-2,8-diazaspiro[4.5]decane-2,8-dicarboxylate (1.2 g, 2.97 mmol) in DCM (10 mL) was added triethylamine (1.24 mL, 8.9 mmol) and methanesulfonyl chloride (0.62 mL, 8.03 mmol). The reaction was stirred at room temperature for 3 h. The mixture was diluted with DCM (40 mL) and washed with water (20 mL). The organic layer was dried over anhydrous Na₂SO₄, filtered and concentrated in vacuo to give the title compound (1.3 g, 91%) as yellow oil.

### Step 2: 2-benzyl 8-tert-butyl 3-(azidomethyl)-2,8-diazaspiro[4.5]decane-2,8-dicarboxylate

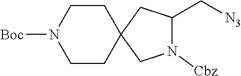 [SMILES: CC(C)(C)OC(=O)N1CCC2(CC1)CC(CN=[N+]=[N-])N(C(=O)OCc1ccccc1)C2]

To a solution of 2-benzyl 8-tert-butyl 3-(((methylsulfonyl)oxy)methyl)-2,8-diazaspiro[4.5]decane-2,8-dicarboxylate (1.3 g, 2.69 mmol) in DMF (15 mL) was added sodium azide (320 mg, 4.92 mmol). The mixture was stirred at 60° C. for 16 h. After cooling to room temperature, the mixture was poured into water and extracted with EtOAc (30 mL×3). The combined organic layers were washed with brine (10 mL), dried over anhydrous Na₂SO₄, filtered and concentrated in vacuo to give the title compound (1.1 g, 95%) as a light yellow solid. LCMS (ESI) m/z: 430.2 [M+H]⁺.

### Step 3: 2-benzyl 8-tert-butyl 3-(aminomethyl)-2,8-diazaspiro[4.5]decane-2,8-dicarboxylate

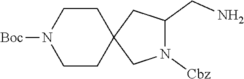 [SMILES: CC(C)(C)OC(=O)N1CCC2(CC1)CC(CN)N(C(=O)OCc1ccccc1)C2]

To a solution of 2-benzyl 8-tert-butyl 3-(azidomethyl)-2,8-diazaspiro[4.5]decane-2,8-dicarboxylate (0.9 g, 2.1 mmol) in THF (10 mL) was added triphenylphosphine (824 mg, 3.14 mmol) and water (0.76 mL, 41.91 mmol). The mixture was stirred at 70° C. for 3 h. After cooling to room temperature, the reaction was concentrated in vacuo. The residue was purified by silica gel chromatography (solvent gradient: 0-5% MeOH in DCM) to give the title compound (0.6 g, 71%) as a green solid. LCMS (ESI) m/z: 404.5 [M+H]⁺.

### Step 4: 2-benzyl 8-tert-butyl 3-(methylsulfonamidomethyl)-2,8-diazaspiro[4.5]decane-2,8-dicarboxylate

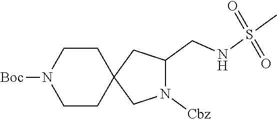 [SMILES: CC(C)(C)OC(=O)N1CCC2(CC1)CC(CNS(C)(=O)=O)N(C(=O)OCc1ccccc1)C2]

To a solution of 2-benzyl 8-tert-butyl 3-(aminomethyl)-2,8-diazaspiro[4.5]decane-2,8-dicarboxylate (300 mg, 0.74 mmol) in DCM (5 mL) was added triethylamine (0.31 mL, 2.23 mmol) and methanesulfonyl chloride (0.16 mL, 2.01 mmol). The solution was stirred at room temperature for 6 h. The mixture was diluted with DCM (40 mL) and washed with water (20 mL). The organic layer was dried over anhydrous Na₂SO₄, filtered and concentrated in vacuo to give the title compound (350 mg, 98%) as yellow oil. LCMS (ESI) m/z: 382.0 [M-100+H]⁺.

### Step 5: benzyl 3-(methylsulfonamidomethyl)-8-(2-(pyridin-4-yl)pyrido[3,4-d]pyrimidin-4-yl)-2,8-diazaspiro[4.5]decane-2-carboxylate

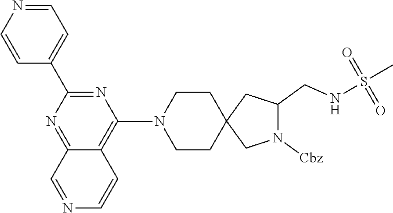 [SMILES: CS(=O)(=O)NCC1CC2(CCN(c3nc(-c4ccncc4)nc4cnccc34)CC2)CN1C(=O)OCc1ccccc1]

Following the procedure described in Example 206, step 3-4 and making non-critical variations as required to replace tert-butyl 2-cyclopentyl-3-(hydroxymethyl)-2,8-diazaspiro[4.5]decane-8-carboxylate with 2-benzyl 8-tert-butyl 3-(methylsulfonamidomethyl)-2,8-diazaspiro[4.5]decane-2,8-dicarboxylate, the title compound was obtained (310 mg, 71%) as a yellow solid. LCMS (ESI) m/z: 588.2 [M+H]⁺.

### Step 6: N-((8-(2-(pyridin-4-yl)pyrido[3,4-d]pyrimidin-4-yl)-2,8-diazaspiro[4.5]decan-3-yl)methyl)methanesulfonamide formate

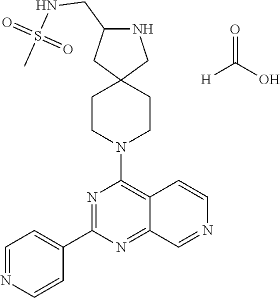 [SMILES: CS(=O)(=O)NCC1CC2(CCN(c3nc(-c4ccncc4)nc4cnccc34)CC2)CN1 ; O=CO]

Following the procedure described in Example 216, step 4 and making non-critical variations as required to replace 2-benzyl 8-tert-butyl 3-(fluoromethyl)-2,8-diazaspiro[4.5]decane-2,8-dicarboxylate with 2-benzyl 8-tert-butyl 3-(methylsulfonamidomethyl)-2,8-diazaspiro[4.5]decane-2,8-dicarboxylate, the title compound was obtained (10 mg, 8%) as a white solid. ¹H NMR (400 MHz, DMSO-d₆) δ 9.26 (s, 1H), 8.77 (d, J=6.0 Hz, 2H), 8.59 (d, J=5.6 Hz, 1H), 8.33 (d, J=6.0 Hz, 2H), 8.27 (s, 1H), 7.90 (d, J=6.0 Hz, 1H), 7.24 (s, 1H), 4.03-3.91 (m, 4H), 3.50-3.41 (m, 1H), 3.10-3.06 (m, 2H), 2.94 (s, 3H), 2.93-2.88 (m, 2H), 2.03-1.96 (m, 1H), 1.82-1.72 (m, 4H), 1.46-1.38 (m, 1H). LCMS (ESI) m/z: 476.1 [M+Na]⁺.

### Example 225

### 5-Bromo-2-(pyridin-4-yl)-4-(2,8-diazaspiro[4.5]decan-8-yl)pyrido[3,4-d]pyrimidine (Compound 225) formate

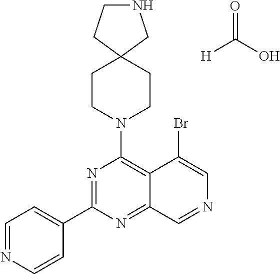 [SMILES: Brc1cncc2nc(-c3ccncc3)nc(N3CCC4(CCNC4)CC3)c12 ; O=CO]

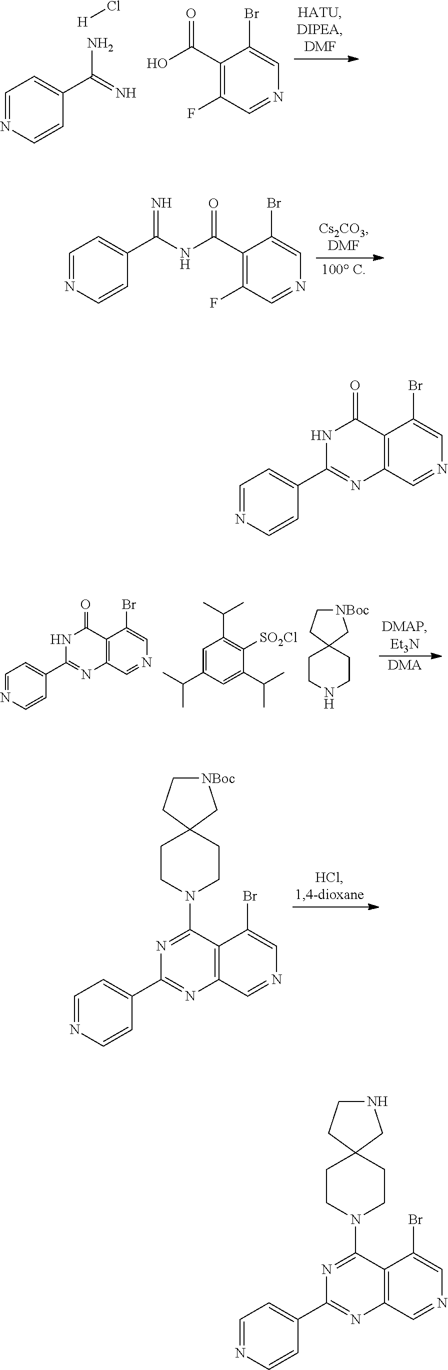

### Step 1: 3-Bromo-5-fluoro-N-(imino(pyridin-4-yl)methyl)isonicotinamide

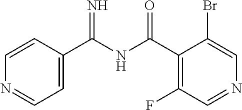 [SMILES: N=C(NC(=O)c1c(F)cncc1Br)c1ccncc1]

Pyridine-4-carboxamidine, hydrochloride (1.38 g, 8.75 mmol) and 3-bromo-5-fluoro-pyridine-4-carboxylic acid (2.0 g, 9.1 mmol) were dissolved in DMF (45 mL) with di-iso-propylethylamine (4.75 mL, 27 mmol). Finally, HATU (3.63 g, 9.55 mmol) was added and the reaction mixture was stirred at room temperature for 16 hours. A saturated solution of sodium bicarbonate (80 mL) was added to reaction mixture and it was extracted 3 times with a 2:8 mixture of iPrOH—CHCl3 (3×50 mL). The organic layers were combined, thoroughly washed with water, brine, dried with anhydrous sodium sulfate, filtered and concentrated to dryness. The crude residue was purified by trituration in MeOH. The off-white precipitate was filtered, rinsed with MeOH and dried to provide 3-bromo-5-fluoro-N-(imino(pyridin-4-yl)methyl)isonicotinamide (1.93 g, 5.76 mmol, 66% yield) as a beige solid. LCMS (ESI) m/z: 323.0/325.0 (Br pattern) [M+H]⁺.

### Step 2: 5-Bromo-2-(pyridin-4-yl)pyrido[3,4-d]pyrimidin-4-ol

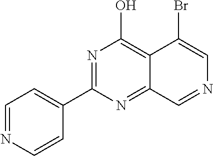 [SMILES: Oc1nc(-c2ccncc2)nc2cncc(Br)c12]

3-Bromo-5-fluoro-N-(imino(pyridin-4-yl)methyl)isonicotinamide (1.93 g, 5.96 mmol) was dissolved in DMF (15 mL) and cesium carbonate (3.9 g, 11.9 mmol) was added. The reaction mixture was stirred at 100° C. for 2 hours. Once conversion completed, the reaction mixture was cooled to room temperature and added dropwise to a stirring solution of NH₄Cl (sat) diluted 1:1 with water (150 mL total). An off-white precipitate formed and was filtered and rinsed with water and acetonitrile. The solid was dried to provide 5-bromo-2-(pyridin-4-yl)pyrido[3,4-d]pyrimidin-4 (3H)-one (1.72 g, 5.66 mmol, 95% yield) as an off white solid. LCMS (ESI) m/z: 302.9/304.9 (Br pattern) [M+H]⁺.

### Step 3: tert-Butyl 8-(5-bromo-2-(pyridin-4-yl)pyrido[3,4-d]pyrimidin-4-yl)-2,8-diazaspiro[4.5]decane-2-carboxylate

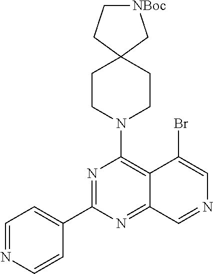 [SMILES: CC(C)(C)OC(=O)N1CCC2(CCN(c3nc(-c4ccncc4)nc4cncc(Br)c34)CC2)C1]

A solution of 5-bromo-2-(4-pyridyl)pyrido[3,4-d]pyrimidin-4-ol (500 mg, 1.7 mmol), 4-dimethylaminopyridine (20.1 mg, 0.2 mmol) N,N-di-iso-propylethylamine (0.86 mL, 5.0 mmol) and 2,4,6-triisopropylbenzenesulfonyl chloride (600 mg, 2.0 mmol) in DMA (5 mL) was stirred at 23° C. for 30 minutes. tert-Butyl 2,8-diazaspiro[4.5]decane-2-carboxylate (475 mg, 2.0 mmol) was added and the mixture was stirred at 23° C. for 3 hours. A saturated solution of NH₄Cl (25 mL) and EtOAc (40 mL) were added. The layers were separated and the organic layer was washed with water (30 mL), brine (30 mL), dried over Na₂SO₄, filtered and concentrated to a brown oil. The crude oil was purified by flash chromatography on silica gel (SiO₂, 0-15% MeOH in DCM) to provide tert-butyl 8-[5-bromo-2-(4-pyridyl)pyrido[3,4-d]pyrimidin-4-yl]-2,8-diazaspiro[4.5]decane-2-carboxylate (505 mg, 0.986 mmol, 58% yield) as a brown gum. UPLCMS (ESI) m/z: 525/527 (Br pattern) [M+H]⁺.

### Step 4: 5-Bromo-2-(pyridin-4-yl)-4-(2,8-diazaspiro[4.5]decan-8-yl)pyrido[3,4-d]pyrimidine formate

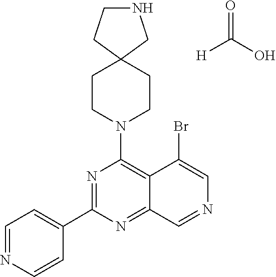 [SMILES: Brc1cncc2nc(-c3ccncc3)nc(N3CCC4(CCNC4)CC3)c12 ; O=CO]

Following the procedure described in Example 199, Step 8 and making non-critical variations as required to replace the substrate with tert-butyl 8-[5-bromo-2-(4-pyridyl)pyrido[3,4-d]pyrimidin-4-yl]-2,8-diazaspiro[4.5]decane-2-carboxylate was obtained the titled compound as a yellow solid (13.4 mg, 0.024 mmol, 83% yield). ¹H NMR (400 MHz, DMSO-d₆) δ 9.19 (s, 1H), 8.79 (dd, J=4.5, 1.5 Hz, 2H), 8.75 (s, 1H), 8.37 (s, 1H), 8.31 (dd, J=4.5, 1.5 Hz, 2H), 3.79-3.73 (m, 4H), 3.22-3.05 (m, 4H), 1.92-1.58 (m, 6H). UPLCMS (ESI) m/z: 425/427 (Br pattern) [M+H]⁺.

### Example 226

### 6-benzyl-2-(3-methyl-1H-pyrazol-4-yl)-4-(2-methyl-2,8-diazaspiro[4.5]decan-8-yl)pyrido[3,4-d]pyrimidine (Compound 226) formate

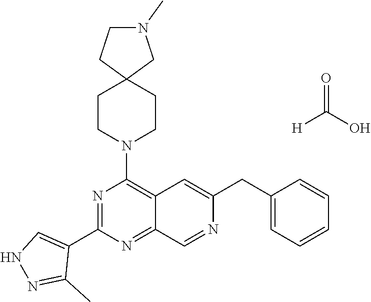 [SMILES: Cc1n[nH]cc1-c1nc(N2CCC3(CCN(C)C3)CC2)c2cc(Cc3ccccc3)ncc2n1 ; O=CO]

### Step 1: tert-butyl 8-(6-(hydroxy(phenyl)methyl)-2-(3-methyl-1-((2-(trimethylsilyl)ethoxy)methyl)-1H-pyrazol-4-yl)pyrido[3,4-d]pyrimidin-4-yl)-2,8-diazaspiro[4.5]decane-2-carboxylate

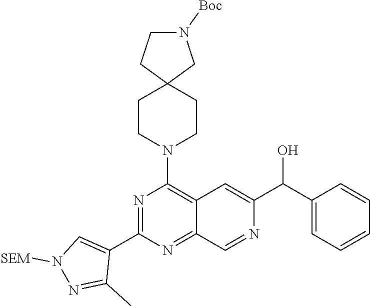 [SMILES: Cc1nn(COCC[Si](C)(C)C)cc1-c1nc(N2CCC3(CCN(C(=O)OC(C)(C)C)C3)CC2)c2cc(C(O)c3ccccc3)ncc2n1]

To a solution of tert-butyl 8-(6-formyl-2-(3-methyl-1-((2-(trimethylsilyl)ethoxy)methyl)-1H-pyrazol-4-yl)pyrido[3,4-d]pyrimidin-4-yl)-2,8-diazaspiro[4.5]decane-2-carboxylate (400 mg, 0.66 mmol) in THF (5 mL) was added phenylmagnesium bromide (0.33 mL, 0.99 mmol) at −78° C. Then the mixture was stirred at 0° C. for 2 h under nitrogen atmosphere. The mixture was quenched with sat. aq. NH₄Cl (10 mL), and extracted with EtOAc (40 mL×2). The combined organic layers were washed with brine (20 mL), dried over anhydrous Na₂SO₄, filtered and concentrated in vacuo. The residue was purified by silica gel chromatography (solvent gradient: 0-70% EtOAc in petroleum ether) to give the title compound (320 mg, 71%) as a yellow solid. LCMS (ESI) m/z: 686.4 [M+H]⁺.

### Step 2: 6-benzyl-2-(3-methyl-1H-pyrazol-4-yl)-4-(2-methyl-2,8-diazaspiro[4.5]decan-8-yl)pyrido[3,4-d]pyrimidine formate

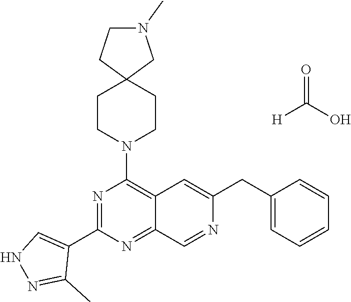 [SMILES: Cc1n[nH]cc1-c1nc(N2CCC3(CCN(C)C3)CC2)c2cc(Cc3ccccc3)ncc2n1 ; O=CO]

Following the procedure described in Example 194, Step 2-5 and making non-critical variations as required to replace tert-butyl 8-(2-(3-methyl-1-((2-(trimethylsilyl)ethoxy)methyl)-1H-pyrazol-4-yl)-6-(2,2,2-trifluoro-1-hydroxyethyl)pyrido[3,4-d]pyrimidin-4-yl)-2,8-diazaspiro[4.5]decane-2-carboxylate with tert-butyl 8-(6-(hydroxy(phenyl)methyl)-2-(3-methyl-1-((2-(trimethylsilyl)ethoxy)methyl)-1H-pyrazol-4-yl)pyrido[3,4-d]pyrimidin-4-yl)-2,8-diazaspiro[4.5]decane-2-carboxylate, the title compound was obtained as a white solid. ¹H NMR (400 MHz, DMSO-d₆) δ 9.02 (s, 1H), 8.21 (s, 1H), 8.12 (s, 1H), 7.51 (s, 1H), 7.34-7.29 (m, 4H), 7.25-7.19 (m, 1H), 4.24 (s, 2H), 3.75-3.68 (m, 4H), 2.94 (t, J=6.8 Hz, 2H), 2.80 (s, 2H), 2.64 (s, 3H), 2.53 (s, 3H), 1.85-1.77 (m, 2H), 1.76-1.63 (m, 4H). LCMS (ESI) m/z: 454.2 [M+H]⁺.

### Example 227

### 2-(3-methyl-1H-pyrazol-4-yl)-4-(2-methyl-2,8-diazaspiro[4.5]decan-8-yl)-6-(pyridin-3-ylmethyl)pyrido[3,4-d]pyrimidine (Compound 227)

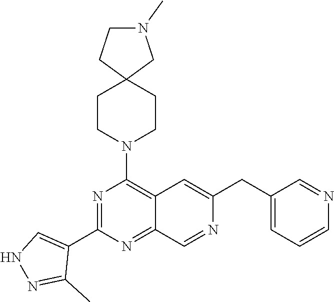 [SMILES: Cc1n[nH]cc1-c1nc(N2CCC3(CCN(C)C3)CC2)c2cc(Cc3cccnc3)ncc2n1]

### Step 1: tert-butyl 8-(6-(hydroxy(pyridin-3-yl)methyl)-2-(3-methyl-1-((2-(trimethylsilyl)ethoxy)methyl)-1H-pyrazol-4-yl)pyrido[3,4-d]pyrimidin-4-yl)-2,8-diazaspiro[4.5]decane-2-carboxylate

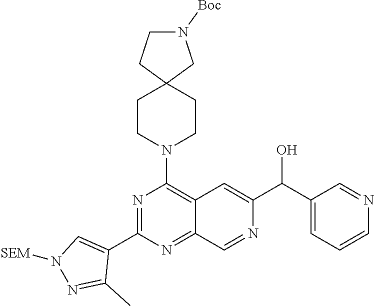 [SMILES: Cc1nn(COCC[Si](C)(C)C)cc1-c1nc(N2CCC3(CCN(C(=O)OC(C)(C)C)C3)CC2)c2cc(C(O)c3cccnc3)ncc2n1]

To a solution of 3-iodopyridine (202 mg, 0.99 mmol) in THF (10 mL) was added isopropylmagnesium chloride lithium chloride complex solution 1.3 M in THF (0.76 mL, 0.99 mmol) at −78° C. The reaction was stirred at −78° C. for 0.5 h, then tert-butyl 8-(6-formyl-2-(3-methyl-1-((2-(trimethylsilyl)ethoxy)methyl)-1H-pyrazol-4-yl)pyrido[3,4-d]pyrimidin-4-yl)-2,8-diazaspiro[4.5]decane-2-carboxylate (0.3 g, 0.49 mmol) was added. The mixture was stirred at 0° C. for additional 2 h. The reaction was quenched with sat. aq. NH₄Cl (5 mL) and extracted with EtOAc (30 mL×2). The combined organic layers were dried over anhydrous Na₂SO₄, filtered and concentrated in vacuo. The crude residue was purified by silica gel chromatography (solvent gradient: 0-10% MeOH in DCM) to give the title compound (150 mg, 38%) as yellow oil. LCMS (ESI) m/z: 687.4 [M+H]⁺.

### Step 2: tert-butyl 8-(6-(acetoxy(pyridin-3-yl)methyl)-2-(3-methyl-1-((2-(trimethylsilyl)ethoxy)methyl)-1H-pyrazol-4-yl)pyrido[3,4-d]pyrimidin-4-yl)-2,8-diazaspiro[4.5]decane-2-carboxylate

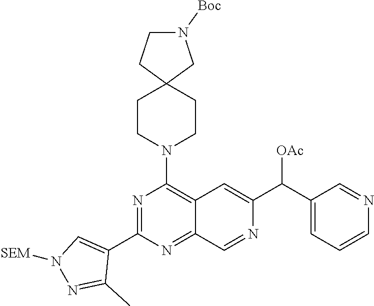 [SMILES: CC(=O)OC(c1cccnc1)c1cc2c(N3CCC4(CCN(C(=O)OC(C)(C)C)C4)CC3)nc(-c3cn(COCC[Si](C)(C)C)nc3C)nc2cn1]

To a solution of 4-dimethylaminopyridine (44 mg, 0.36 mmol) and tert-butyl 8-(6-(hydroxy(pyridin-3-yl)methyl)-2-(3-methyl-1-((2-(trimethylsilyl)ethoxy)methyl)-1H-pyrazol-4-yl)pyrido[3,4-d]pyrimidin-4-yl)-2,8-diazaspiro[4.5]decane-2-carboxylate (250 mg, 0.36 mmol) in pyridine (6 mL) was added acetic anhydride (0.15 mL, 1.06 mmol). The mixture was stirred at room temperature for 2 h. The reaction was quenched with sat. aq. NH₄Cl (5 mL) and extracted with EtOAc (30 mL×2). The combined organic layers were dried over anhydrous Na₂SO₄, filtered and concentrated in vacuo. The crude residue was purified by silica gel chromatography (solvent gradient: 0-10% MeOH in DCM) to give the title compound (240 mg, 91%) as a yellow solid. LCMS (ESI) m/z: 729.4 [M+H]⁺.

### Step 3: 2-(3-methyl-1H-pyrazol-4-yl)-4-(2-methyl-2,8-diazaspiro[4.5]decan-8-yl)-6-(pyri din-3-ylmethyl)pyrido[3,4-d]pyrimidine

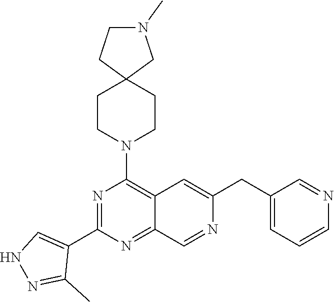

Following the procedure described in Example 194, Step 3-5 and making non-critical variations as required to replace tert-butyl 8-(2-(3-methyl-1-((2-(trimethylsilyl)ethoxy)methyl)-1H-pyrazol-4-yl)-6-(2,2,2-trifluoro-1-((methylsulfonyl)oxy)ethyl)pyrido[3,4-d]pyrimidin-4-yl)-2,8-diazaspiro[4.5]decane-2-carboxylate with tert-butyl 8-(6-(acetoxy(pyridin-3-yl)methyl)-2-(3-methyl-1-((2-(trimethylsilyl)ethoxy)methyl)-1H-pyrazol-4-yl)pyrido[3,4-d]pyrimidin-4-yl)-2,8-diazaspiro[4.5]decane-2-carboxylate, the title compound was obtained as a white solid. ¹H NMR (400 MHz, DMSO-d₆) δ 12.81 (s, 1H), 9.02 (s, 1H), 8.58 (d, J=1.6 Hz, 1H), 8.44-8.42 (m, 1H), 8.11 (s, 1H), 7.73 (d, J=8.0 Hz, 1H), 7.62 (s, 1H), 7.35-7.30 (m, 2H), 4.27 (s, 2H), 3.82-3.65 (m, 4H), 2.85-2.65 (m, 2H), 2.64 (s, 3H), 2.51-2.44 (m, 2H), 2.41 (s, 3H), 1.76-1.69 (m, 6H). LCMS (ESI) m/z: 455.2 [M+H]⁺.

### Examples 228 & 229

### (1R,2R)-2-(8-(2-(5-methyl-1H-pyrazol-4-yl)pyrido[3,4-d]pyrimidin-4-yl)-2,8-diazaspiro[4.5]decan-2-yl)cyclobutanol & (1S,2S)-2-(8-(2-(5-methyl-1H-pyrazol-4-yl)pyrido[3,4-d]pyrimidin-4-yl)-2,8-diazaspiro[4.5]decan-2-yl)cyclobutanol Compounds 228 and 229)

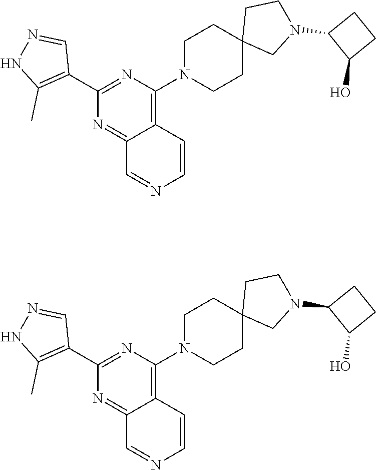 [SMILES: Cc1[nH]ncc1-c1nc(N2CCC3(CC2)CCN([C@@H]2CC[C@H]2O)C3)c2ccncc2n1 ; Cc1[nH]ncc1-c1nc(N2CCC3(CC2)CCN([C@H]2CC[C@@H]2O)C3)c2ccncc2n1]

### Step 1: (trans)-2-(8-(2-(5-methyl-1H-pyrazol-4-yl)pyrido[3,4-d]pyrimidin-4-yl)-2,8-diazaspiro[4.5]decan-2-yl)cyclobutanol & (cis)-2-(8-(2-(5-methyl-1H-pyrazol-4-yl)pyrido[3,4-d]pyrimidin-4-yl)-2,8-diazaspiro[4.5]decan-2-yl)cyclobutanol

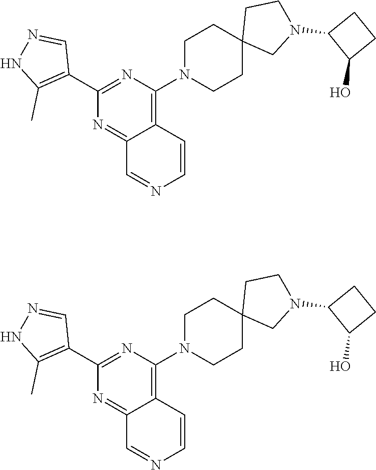 [SMILES: Cc1[nH]ncc1-c1nc(N2CCC3(CC2)CCN([C@@H]2CC[C@H]2O)C3)c2ccncc2n1 ; Cc1[nH]ncc1-c1nc(N2CCC3(CC2)CCN([C@@H]2CC[C@@H]2O)C3)c2ccncc2n1]

Following the procedure described in Example 178, Step 1-2 and making non-critical variations as required to replace 4-(2,8-diazaspiro[4.5]decan-8-yl)-2-(4-pyridyl)pyrido[3,4-d]pyrimidine hydrochloride with 2-(3-methyl-1H-pyrazol-4-yl)-4-(2,8-diazaspiro[4.5]decan-8-yl)pyrido[3,4-d]pyrimidine hydrochloride, the title compounds were obtained (trans 36 mg & cis 35 mg) both as a white solid. LCMS (ESI) m/z: 420.2 [M+H]⁺. Trans isomer: ¹H NMR (400 MHz, DMSO-d₆) δ 12.86 (s, 1H), 9.07 (s, 1H), 8.44 (d, J=5.6 Hz, 1H), 8.07 (s, 1H), 7.75 (d, J=5.6 Hz, 1H), 5.07 (d, J=7.2 Hz, 1H), 3.90-3.69 (m, 5H), 2.72-2.63 (m, 3H), 2.60-2.54 (m, 3H), 2.47-2.45 (m, 1H), 2.41-2.37 (m, 1H), 2.01-1.93 (m, 1H), 1.75-1.59 (m, 7H), 1.43-1.33 (m, 1H), 1.22-1.14 (m, 1H). LCMS (ESI) m/z: 420.2 [M+H]⁺. Cis isomer: ¹H NMR (400 MHz, DMSO-d₆) δ 12.86 (s, 1H), 9.07 (s, 1H), 8.44 (d, J=5.6 Hz, 1H), 8.07 (s, 1H), 7.76 (d, J=5.6 Hz, 1H), 4.75-4.55 (m, 1H), 4.10-4.00 (m, 1H), 3.90-3.70 (m, 4H), 2.84-2.76 (m, 1H), 2.69-2.63 (m, 3H), 2.60-2.54 (m, 2H), 2.44-2.37 (m, 2H), 2.04-1.96 (m, 1H), 1.86-1.78 (m, 2H), 1.77-1.60 (m, 7H). LCMS (ESI) m/z: 420.2 [M+H]⁺.

### Step 2: (1R,2R)-2-(8-(2-(5-methyl-1H-pyrazol-4-yl)pyrido[3,4-d]pyrimidin-4-yl)-2,8-diazaspiro[4.5]decan-2-yl)cyclobutanol & (1S,2S)-2-(8-(2-(5-methyl-1H-pyrazol-4-yl)pyrido[3,4-d]pyrimidin-4-yl)-2,8-diazaspiro[4.5]decan-2-yl)cyclobutanol

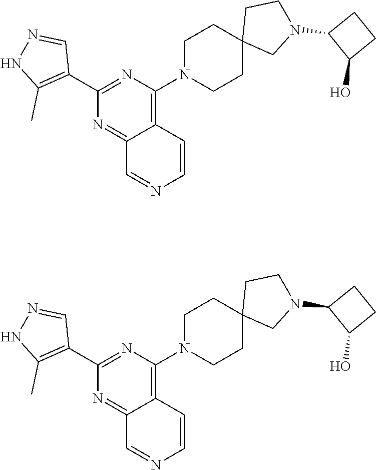 [SMILES: Cc1[nH]ncc1-c1nc(N2CCC3(CC2)CCN([C@@H]2CC[C@H]2O)C3)c2ccncc2n1 ; Cc1[nH]ncc1-c1nc(N2CCC3(CC2)CCN([C@H]2CC[C@@H]2O)C3)c2ccncc2n1]

(trans)-2-(8-(2-(5-Methyl-1H-pyrazol-4-yl)pyrido[3,4-d]pyrimidin-4-yl)-2,8-diazaspiro[4.5]decan-2-yl)cyclobutanol (36 mg, 0.09 mmol) was separated by using chiral SFC (Chiralpak IG (250 mm*30 mm, 10 um), Supercritical CO₂/EtOH+0.1% NH₄OH=45/55; 80 mL/min) to give (1R,2R)-2-(8-(2-(5-methyl-1H-pyrazol-4-yl)pyrido[3,4-d]pyrimidin-4-yl)-2,8-diazaspiro[4.5]decan-2-yl)cyclobutanol (14 mg, first peak) and (1S,2S)-2-(8-(2-(5-methyl-1H-pyrazol-4-yl)pyrido[3,4-d]pyrimidin-4-yl)-2,8-diazaspiro[4.5]decan-2-yl)cyclobutanol (14 mg, second peak) both as white solid. Absolute configuration was arbitrarily assigned to each enantiomer. Example 228 (first peak): ¹H NMR (400 MHz, DMSO-d₆) δ 12.86 (s, 1H), 9.07 (s, 1H), 8.44 (d, J=5.6 Hz, 1H), 8.07 (s, 1H), 7.75 (d, J=5.6 Hz, 1H), 5.07 (d, J=7.2 Hz, 1H), 3.90-3.69 (m, 5H), 2.72-2.63 (m, 3H), 2.60-2.54 (m, 3H), 2.47-2.45 (m, 1H), 2.41-2.37 (m, 1H), 2.01-1.93 (m, 1H), 1.75-1.59 (m, 7H), 1.43-1.33 (m, 1H), 1.22-1.14 (m, 1H). LCMS (ESI) m/z: 420.1 [M+H]⁺. Example 229 (second peak): ¹H NMR (400 MHz, DMSO-d₆) δ 12.86 (s, 1H), 9.07 (s, 1H), 8.44 (d, J=5.6 Hz, 1H), 8.07 (s, 1H), 7.75 (d, J=5.6 Hz, 1H), 5.07 (d, J=7.2 Hz, 1H), 3.90-3.70 (m, 5H), 2.72-2.63 (m, 3H), 2.62-2.54 (m, 3H), 2.47-2.45 (m, 1H), 2.42-2.36 (m, 1H), 2.00-1.90 (m, 1H), 1.77-1.60 (m, 7H), 1.42-1.36 (m, 1H), 1.22-1.14 (m, 1H). LCMS (ESI) m/z: 420.2 [M+H]⁺

### Examples 230 & 231

### (1S,2R)-2-(8-(2-(5-methyl-1H-pyrazol-4-yl)pyrido[3,4-d]pyrimidin-4-yl)-2,8-diazaspiro[4.5]decan-2-yl)cyclobutanol & (1R,2S)-2-(8-(2-(5-methyl-1H-pyrazol-4-yl)pyrido[3,4-d]pyrimidin-4-yl)-2,8-diazaspiro[4.5]decan-2-yl)cyclobutanol (Compounds 230 and 231)

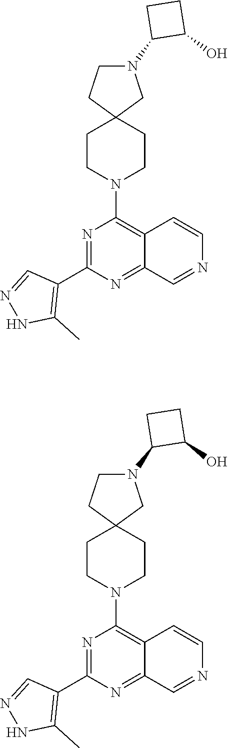

(cis)-2-(8-(2-(5-Methyl-1H-pyrazol-4-yl)pyrido[3,4-d]pyrimidin-4-yl)-2,8-diazaspiro[4.5]decan-2-yl)cyclobutanol (35 mg, 0.08 mmol) was separated by using chiral SFC (Chiralpak IG (250 mm*30 mm, 10 um), Supercritical CO₂/EtOH+0.1% NH₄OH=45/55; 80 mL/min) to give (1S,2R)-2-(8-(2-(5-methyl-1H-pyrazol-4-yl)pyrido[3,4-d]pyrimidin-4-yl)-2,8-diazaspiro[4.5]decan-2-yl)cyclobutanol (12 mg, first peak) and (1R,2S)-2-(8-(2-(5-methyl-1H-pyrazol-4-yl)pyrido[3,4-d]pyrimidin-4-yl)-2,8-diazaspiro[4.5]decan-2-yl)cyclobutanol (12 mg, second peak) both as white solid. Absolute configuration was arbitrarily assigned to each enantiomer. Example 230 (first peak): ¹H NMR (400 MHz, DMSO-d₆) δ 12.86 (s, 1H), 9.07 (s, 1H), 8.44 (d, J=5.6 Hz, 1H), 8.07 (s, 1H), 7.76 (d, J=5.6 Hz, 1H), 4.75-4.55 (m, 1H), 4.10-4.00 (m, 1H), 3.90-3.70 (m, 4H), 2.84-2.76 (m, 1H), 2.69-2.63 (m, 3H), 2.60-2.54 (m, 2H), 2.44-2.37 (m, 2H), 2.04-1.96 (m, 1H), 1.86-1.78 (m, 2H), 1.77-1.60 (m, 7H). LCMS (ESI) m/z: 420.2 [M+H]⁺. Example 231 (second peak): ¹H NMR (400 MHz, DMSO-d₆) δ 12.86 (s, 1H), 9.07 (s, 1H), 8.44 (d, J=5.6 Hz, 1H), 8.36-8.00 (m, 1H), 7.76 (d, J=5.6 Hz, 1H), 4.75-4.55 (m, 1H), 4.10-4.00 (m, 1H), 3.90-3.72 (m, 4H), 2.88-2.77 (m, 1H), 2.73-2.66 (m, 3H), 2.62-2.55 (m, 2H), 2.45-2.37 (m, 2H), 2.03-1.96 (m, 1H), 1.87-1.78 (m, 2H), 1.77-1.60 (m, 7H). LCMS (ESI) m/z: 420.2 [M+H]⁺.

### Example 232

### 8-chloro-2-(3-methyl-1H-pyrazol-4-yl)-4-(2,8-diazaspiro[4.5]decan-8-yl)pyrido[3,4-d]pyrimidine (Compound 232)

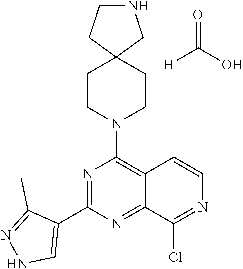

Following the procedure described in Example 142, step 2-3 and making non-critical variations as required to replace 3-aminopyridine-4-carboxamide with 3-amino-2-chloroisonicotinamide (prepared according to methods known in the art, for example, as described in WO2020/239999A1), the title compound was obtained (64 mg 34%) as a white solid. ¹H NMR (400 MHz, DMSO-d₆) δ 8.36 (s, 1H), 8.23 (d, J=5.6 Hz, 1H), 8.16 (s, 1H), 7.77 (d, J=5.6 Hz, 1H), 3.92-3.79 (m, 4H), 3.24 (t, J=7.2 Hz, 2H), 3.05 (s, 2H), 2.70 (s, 3H), 1.88 (t, J=7.2 Hz, 2H), 1.77 (m, 4H). LCMS (ESI) m/z: 384.0 [M+H]⁺.

### Example 233

### 5-Methoxy-2-(pyridin-4-yl)-4-(2,8-diazaspiro[4.5]decan-8-yl)pyrido[3,4-d]pyrimidine (Compound 233) formate

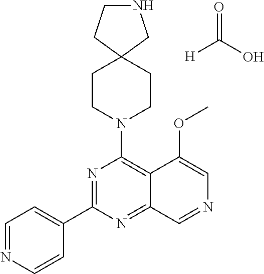

### Step 1: 3,5-difluoro-N-(imino(pyridin-4-yl)methyl)isonicotinamide

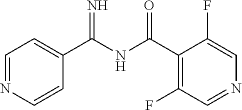 [SMILES: N=C(NC(=O)c1c(F)cncc1F)c1ccncc1]

To a solution of 3,5-difluoroisonicotinic acid (175 g, 1.1 mol) in DMF (2.1 L) was added HATU (460 g, 1.21 mol) and N,N-diisopropylethylamine (545.4 mL, 3.3 mol) at room temperature. After stirring for 5 min, isonicotinimidamide hydrochloride (182 g, 1.16 mol) was added to this reaction mixture. The resulting mixture was stirred for 5 h at room temperature. The reaction mixture was added dropwise to water (4.2 L) and stirred for 30 min. A white precipitate was formed and filtered off, the filter cake was washed with water (500 mL×2), petroleum ether (500 mL×2) and dried in vacuo to give the title compound (124 g, 43%) as a white solid. LCMS (ESI) m/z: 263.1 [M+H]⁺.

### Step 2: 5-fluoro-2-(pyridin-4-yl)pyrido[3,4-d]pyrimidin-4-ol

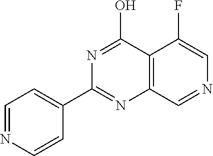 [SMILES: Oc1nc(-c2ccncc2)nc2cncc(F)c12]

To a solution of 3,5-difluoro-N-(imino(pyridin-4-yl)methyl)isonicotinamide (124 g, 472.9 mmol) in DMF (900 mL) was added Cs₂CO₃ (185 g, 567.5 mmol). The mixture was stirred at 100° C. for 3 h. After cooling to room temperature, the reaction mixture was added water (1.8 L) and stirred for 30 min. The mixture was adjusted to pH 5 with AcOH, then stirred for 30 min. A white precipitate was formed and filtered off, the filter cake was washed with water (400 mL×2), petroleum ether (400 mL×2) and dried in vacuo to give the title compound (91 g, 80%) as a white solid. LCMS (ESI) m/z: 242.6 [M+H]⁺.

### Step 3: 5-methoxy-2-(pyridin-4-yl)pyrido[3,4-d]pyrimidin-4-ol

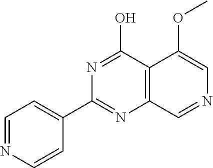 [SMILES: COc1cncc2nc(-c3ccncc3)nc(O)c12]

To a solution of 5-fluoro-2-(pyridin-4-yl)pyrido[3,4-d]pyrimidin-4-ol (91 g, 375.7 mmol) in DMF (600 mL) was added sodium methoxide (60.9 g, 1.13 mol). The mixture was stirred at 40° C. for 3 h. After cooling to room temperature, the reaction mixture was added to water (1.2 L) and stirred for 30 min. The mixture was adjusted to pH 5 with AcOH, then stirred for 30 min. A white precipitate was formed and filtered off, the filter cake was washed with water (350 mL×2), petroleum ether (350 mL×2) and dried in vacuo to give the title compound (90 g, 94%) as a white solid. LCMS (ESI) m/z: 254.7 [M+H]⁺.

### Step 4: tert-butyl 8-(5-methoxy-2-(pyridin-4-yl)pyrido[3,4-d]pyrimidin-4-yl)-2,8-diazaspiro[4.5]decane-2-carboxylate

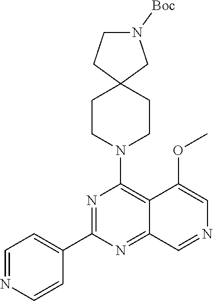 [SMILES: COc1cncc2nc(-c3ccncc3)nc(N3CCC4(CCN(C(=O)OC(C)(C)C)C4)CC3)c12]

A mixture of 5-methoxy-2-(pyridin-4-yl)pyrido[3,4-d]pyrimidin-4-ol (90 g, 354 mmol), PyBOP (221 g, 425 mmol) and triethylamine (148 mL, 1.06 mol) in DMF (900 mL) was stirred at room temperature for 10 min before tert-butyl 2,8-diazaspiro[4.5]decane-2-carboxylate (85.1 g, 354 mmol) was added. The reaction was stirred at room temperature for 16 h. The reaction mixture was quenched with water (1.8 L), extracted with EtOAc (2.5 L×3). The combined organic layers were washed with water (2 L×3) and brine (2 L), dried over anhydrous Na₂SO₄ and concentrated in vacuo. The crude residue was purified by silica gel chromatography (solvent gradient: 0-3% MeOH in DCM) to give the title compound (141 g, 84%) as a yellow solid. LCMS (ESI) m/z: 477.2 [M+H]⁺.

### Step 5: 5-methoxy-2-(pyridin-4-yl)-4-(2,8-diazaspiro[4.5]decan-8-yl)pyrido[3,4-d]pyrimidine hydrochloride

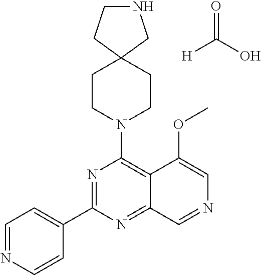

Following the procedure described in Example 199, Step 8 and making non-critical variations as required to replace the substrate with tert-butyl 8-(5-methoxy-2-(pyridin-4-yl)pyrido[3,4-d]pyrimidin-4-yl)-2,8-diazaspiro[4.5]decane-2-carboxylate was obtained the titled compound as an off white solid. ¹H NMR (400 MHz, DMSO-d₆) δ 8.83 (s, 1H), 8.76 (dd, J=4.5, 1.4 Hz, 2H), 8.40 (s, 1H), 8.34 (s, 1H), 8.30 (dd, J=4.5, 1.5 Hz, 2H), 4.08 (s, 3H), 3.78-3.63 (m, 4H), 3.16 (t, J=7.2 Hz, 2H), 2.97 (s, 2H), 1.81 (t, J=7.3 Hz, 2H), 1.77-1.63 (m, 4H). UPLCMS (ESI) m/z: 377.3 [M+H]⁺.

### Examples 234 & 235

### (S)-4-(4-fluoro-2,8-diazaspiro[4.5]decan-8-yl)-2-(pyridin-4-yl)pyrido[3,4-d]pyrimidine & (R)-4-(4-fluoro-2,8-diazaspiro[4.5]decan-8-yl)-2-(pyridin-4-yl)pyrido[3,4-d]pyrimidine (Compounds 234 and 235) trifluoroacetate

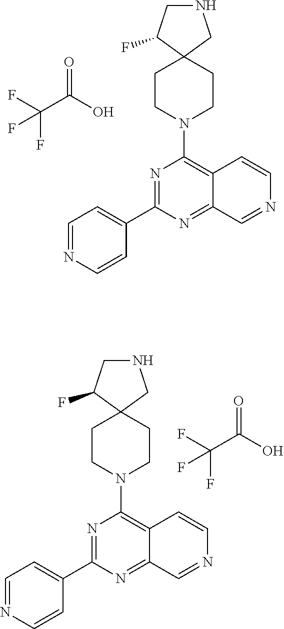

### Step 1: 8-benzyl 2-tert-butyl 4-fluoro-2,8-diazaspiro[4.5]decane-2,8-dicarboxylate

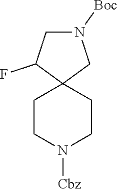 [SMILES: CC(C)(C)OC(=O)N1CC(F)C2(CCN(C(=O)OCc3ccccc3)CC2)C1]

To a solution of 8-benzyl 2-tert-butyl 4-hydroxy-2,8-diazaspiro[4.5]decane-2,8-dicarboxylate (400 mg, 1.02 mmol) in DCM (6 mL) at −78° C. was added diethylaminosulfur trifluoride (0.41 mL, 3.07 mmol) dropwise. The mixture was stirred at 0° C. for 1 h. The reaction was quenched with sat. aq. NaHCO₃ (10 mL) and extracted with DCM (20 mL×2). The combined organic layers were dried over anhydrous Na₂SO₄, filtered and concentrated in vacuo. The crude residue was purified by silica gel chromatography (solvent gradient: 0-30% EtOAc in petroleum ether) to give the title compound (160 mg, crude) as yellow oil that required no further purification. LCMS (ESI) m/z: 293.1 [M+H-100]⁺.

### Step 2: tert-butyl 4-fluoro-2,8-diazaspiro[4.5]decane-2-carboxylate

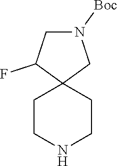 [SMILES: CC(C)(C)OC(=O)N1CC(F)C2(CCNCC2)C1]

To a solution of 8-benzyl 2-tert-butyl 4-fluoro-2,8-diazaspiro[4.5]decane-2,8-dicarboxylate (160 mg, 0.41 mmol) in EtOAc (3 mL) was added 10% palladium on carbon (50 mg). The mixture was stirred at room temperature for 3 h under hydrogen atmosphere (15 psi). The mixture was filtered and the filtrate was concentrated in vacuo to give the title compound (80 mg, crude) as yellow oil that required no further purification. LCMS (ESI) m/z: 259.1 [M+H]⁺.

### Step 3: tert-butyl 4-fluoro-2,8-diazaspiro[4.5]decane-2-carboxylate

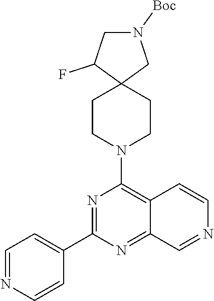 [SMILES: CC(C)(C)OC(=O)N1CC(F)C2(CCN(c3nc(-c4ccncc4)nc4cnccc34)CC2)C1]

Following the procedure described in Example 202 and making non-critical variations as required to replace tert-butyl 2,8-diazaspiro[4.5]decane-2-carboxylate with tert-butyl 4-fluoro-2,8-diazaspiro[4.5]decane-2-carboxylate, the title compound was obtained (30 mg, 21%) as a yellow solid. LCMS (ESI) m/z: 465.1 [M+H]⁺.

### Step 4: (S)-tert-butyl 4-fluoro-8-(2-(pyridin-4-yl)pyrido[3,4-d]pyrimidin-4-yl)-2,8-diazaspiro[4.5]decane-2-carboxylate & (R)-tert-butyl 4-fluoro-8-(2-(pyridin-4-yl)pyrido[3,4-d]pyrimidin-4-yl)-2,8-diazaspiro[4.5]decane-2-carboxylate

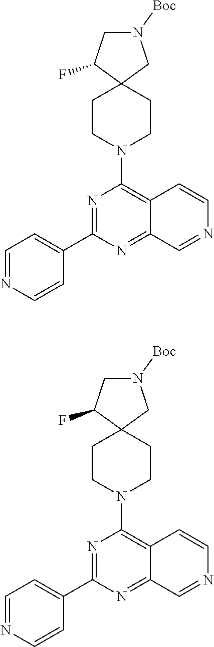 [SMILES: CC(C)(C)OC(=O)N1C[C@@H](F)C2(CCN(c3nc(-c4ccncc4)nc4cnccc34)CC2)C1 ; CC(C)(C)OC(=O)N1C[C@H](F)C2(CCN(c3nc(-c4ccncc4)nc4cnccc34)CC2)C1]

tert-Butyl 4-fluoro-8-[2-(4-pyridyl)pyrido[3,4-d]pyrimidin-4-yl]-2,8-diazaspiro[4.5]decane-2-carboxylate (30 mg, 0.06 mmol) was separated by using chiral SFC (Chiralpak AD (250 mm*30 mm, 10 um), Supercritical CO₂/EtOH+0.1% NH₄OH=55/45; 60 mL/min) to give (S)-tert-butyl 4-fluoro-8-(2-(pyridin-4-yl)pyrido[3,4-d]pyrimidin-4-yl)-2,8-diazaspiro[4.5]decane-2-carboxylate (10 mg, first peak) and (R)-tert-butyl 4-fluoro-8-(2-(pyridin-4-yl)pyrido[3,4-d]pyrimidin-4-yl)-2,8-diazaspiro[4.5]decane-2-carboxylate (11 mg, second peak) both as white solid. Absolute configuration was arbitrarily assigned to each enantiomer. LCMS (ESI) m/z: 465.1 [M+H]⁺.

### Step 5: (S)-4-(4-fluoro-2,8-diazaspiro[4.5]decan-8-yl)-2-(pyridin-4-yl)pyrido[3,4-d]pyrimidine trifluoro acetate & (R)-4-(4-fluoro-2,8-diazaspiro[4.5]decan-8-yl)-2-(pyridin-4-yl)pyrido[3,4-d]pyrimidine trifluoroacetate

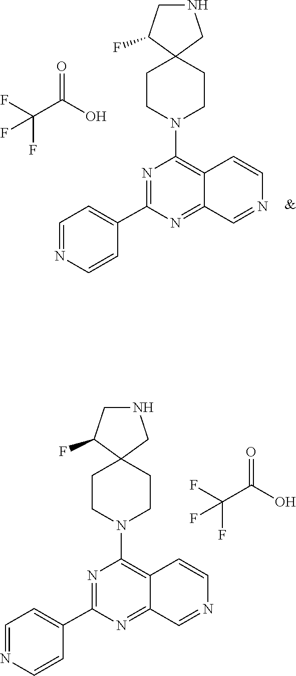

Following the procedure described in Example 157, step 7 and making non-critical variations as required to replace tert-butyl 8-(6-benzyl-2-(pyridin-4-yl)pyrido[3,4-d]pyrimidin-4-yl)-2,8-diazaspiro[4.5]decane-2-carboxylate with (S)-tert-butyl 4-fluoro-8-(2-(pyridin-4-yl)pyrido[3,4-d]pyrimidin-4-yl)-2,8-diazaspiro[4.5]decane-2-carboxylate (first peak from Step 4 above, the absolute configuration was arbitrarily assigned), Compound 234 was obtained (11 mg, 85%) as a yellow solid. ¹H NMR (400 MHz, DMSO-d₆) δ 9.22 (s, 1H), 8.77 (d, J=5.6 Hz, 2H), 8.56 (d, J=5.6 Hz, 1H), 8.32 (d, J=5.6 Hz, 2H), 8.29 (br s, 1H), 8.05 (d, J=6.0 Hz, 1H), 4.45-4.42 (m, 1H), 4.27-4.08 (m, 2H), 3.94-3.89 (m, 2H), 3.31-3.15 (m, 3H), 2.82-2.75 (m, 1H), 2.47-2.39 (m, 1H), 2.34-2.14 (m, 2H), 2.08-1.99 (m, 2H). LCMS (ESI) m/z: 365.3 [M+H]⁺.

Following the procedure described in Example 157, step 7 and making non-critical variations as required to replace tert-butyl 8-(6-benzyl-2-(pyridin-4-yl)pyrido[3,4-d]pyrimidin-4-yl)-2,8-diazaspiro[4.5]decane-2-carboxylate with (R)-tert-butyl 4-fluoro-8-(2-(pyridin-4-yl)pyrido[3,4-d]pyrimidin-4-yl)-2,8-diazaspiro[4.5]decane-2-carboxylate (second peak from Step 4 above, the absolute configuration was arbitrarily assigned), Compound 235 was obtained (9 mg, 71%) as a yellow solid. ¹H NMR (400 MHz, DMSO-d₆) δ 9.22 (s, 1H), 8.77 (d, J=5.6 Hz, 2H), 8.56 (d, J=5.6 Hz, 1H), 8.32-8.28 (m, 3H), 8.05 (d, J=6.0 Hz, 1H), 4.45-4.42 (m, 1H), 4.27-4.08 (m, 2H), 3.94-3.89 (m, 2H), 3.34-3.15 (m, 3H), 2.82-2.75 (m, 1H), 2.47-2.39 (m, 1H), 2.34-2.14 (m, 2H), 2.08-1.98 (m, 2H). LCMS (ESI) m/z: 365.3 [M+H]⁺.

### Example 236

### 2-(3-methyl-1H-pyrazol-4-yl)-4-(2-methyl-2,8-diazaspiro[4.5]decan-8-yl)-6-(pyridin-4-ylmethyl)pyrido[3,4-d]pyrimidine (Compound 236)

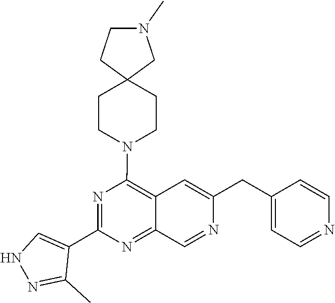 [SMILES: Cc1n[nH]cc1-c1nc(N2CCC3(CCN(C)C3)CC2)c2cc(Cc3ccncc3)ncc2n1]

Following the procedure described in Example 227, Step 1-3 and making non-critical variations as required to replace 3-iodopyridine with 4-iodopyridine, the title compound was obtained as a white solid. ¹H NMR (400 MHz, DMSO-d₆) δ 12.85 (s, 1H), 9.02 (s, 1H), 8.47 (d, J=5.6 Hz, 2H), 8.04 (s, 1H), 7.64 (s, 1H), 7.32 (d, J=5.6 Hz, 2H), 4.26 (s, 2H), 3.82-3.67 (m, 4H), 2.65 (s, 3H), 2.61-2.52 (m, 2H), 2.37 (s, 2H), 2.23 (s, 3H), 1.74-1.61 (m, 6H). LCMS (ESI) m/z: 455.2 [M+H]⁺.

### Example 237

### 2-(3-methyl-1H-pyrazol-4-yl)-4-(2,8-diazaspiro[4.5]decan-8-yl)pyrido[3,4-d]pyrimidin-8-ol (Compound 237)

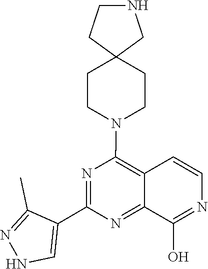 [SMILES: Cc1n[nH]cc1-c1nc(N2CCC3(CCNC3)CC2)c2ccnc(O)c2n1]

### Step 1: tert-butyl 8-(8-hydroxy-2-(3-methyl-1-((2-(trimethylsilyl)ethoxy)methyl)-1H-pyrazol-4-yl)pyrido[3,4-d]pyrimidin-4-yl)-2,8-diazaspiro[4.5]decane-2-carboxylate

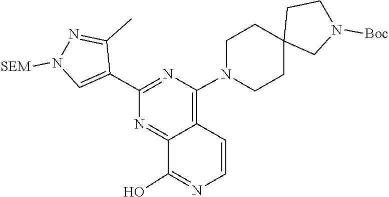 [SMILES: Cc1nn(COCC[Si](C)(C)C)cc1-c1nc(N2CCC3(CCN(C(=O)OC(C)(C)C)C3)CC2)c2ccnc(O)c2n1]

To a solution of tert-butyl 8-(8-chloro-2-(3-methyl-1-((2-(trimethylsilyl)ethoxy)methyl)-1H-pyrazol-4-yl)pyrido[3,4-d]pyrimidin-4-yl)-2,8-diazaspiro[4.5]decane-2-carboxylate (400 mg, 0.65 mmol) and potassium hydroxide (110 mg, 1.95 mmol) in dioxane (5 mL) was added tBuXPhos Pd G3 (52 mg, 0.07 mmol). The mixture was evacuated and backfilled with nitrogen three times, and then heated to 100° C. for 16 h under nitrogen atmosphere. After cooling to room temperature, the mixture was filtered and the filtrate was concentrated in vacuo. The residue was purified by prep-TLC (DCM/MeOH=20:1) to give the title compound (270 mg, 70%) as a yellow solid. LCMS (ESI) m/z: 596.3 [M+H]⁺.

### Step 2:2-(3-methyl-1H-pyrazol-4-yl)-4-(2,8-diazaspiro[4.5]decan-8-yl)pyrido[3,4-d]pyrimidin-8-ol

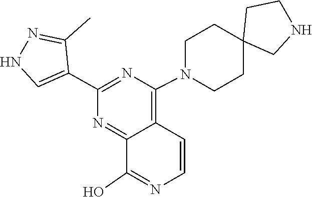 [SMILES: Cc1n[nH]cc1-c1nc(N2CCC3(CCNC3)CC2)c2ccnc(O)c2n1]

Following the procedure described in Example 157, step 7 and making non-critical variations as required to replace tert-butyl 8-(6-benzyl-2-(pyridin-4-yl)pyrido[3,4-d]pyrimidin-4-yl)-2,8-diazaspiro[4.5]decane-2-carboxylate with tert-butyl 8-(8-hydroxy-2-(3-methyl-1-((2-(trimethylsilyl)ethoxy)methyl)-1H-pyrazol-4-yl)pyrido[3,4-d]pyrimidin-4-yl)-2,8-diazaspiro[4.5]decane-2-carboxylate, the title compound (51 mg, 31%) was obtained as a white solid. ¹H NMR (400 MHz, DMSO-d₆) δ 8.07 (s, 1H), 7.13 (d, J=7.2 Hz, 1H), 6.34 (d, J=7.2 Hz, 1H), 3.71-3.62 (m, 2H), 3.59-3.56 (m, 2H), 3.34 (t, J=6.8 Hz, 2H), 3.20 (s, 2H), 2.64 (s, 3H), 1.82-1.78 (m, 1H), 1.76-1.63 (m, 4H), 1.62-1.57 (m, 1H). 3 exchangeable protons not observed (pyrazole NH, amine NH, OH). LCMS (ESI) m/z: 366.3 [M+H]⁺.

### Example 238

### 2-(3-methyl-1H-pyrazol-4-yl)-4-(2-methyl-2,8-diazaspiro[4.5]decan-8-yl)-6-(pyridin-2-ylmethyl)pyrido[3,4-d]pyrimidine (Compound 238)

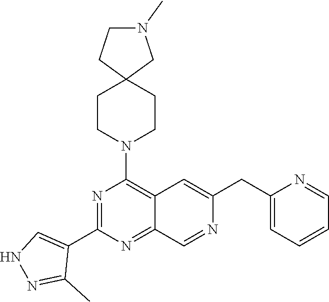 [SMILES: Cc1n[nH]cc1-c1nc(N2CCC3(CCN(C)C3)CC2)c2cc(Cc3ccccn3)ncc2n1]

Following the procedure described in Example 227, Step 1-3 and making non-critical variations as required to replace 3-iodopyridine with 2-bromopyridine, the title compound was obtained as a white solid. ¹H NMR (400 MHz, DMSO-d₆) δ 12.85 (s, 1H), 9.00 (s, 1H), 8.49 (d, J=4.0 Hz, 1H), 8.05 (s, 1H), 7.76-7.71 (m, 1H), 7.59 (s, 1H), 7.35 (d, J=8.0 Hz, 1H), 7.27-7.21 (m, 1H), 4.39 (s, 2H), 3.81-3.66 (m, 4H), 2.65 (s, 3H), 2.51-2.51 (m, 2H), 2.38 (s, 2H), 2.23 (s, 3H), 1.71-1.62 (m, 6H). LCMS (ESI) m/z: 455.2 [M+H].

### Example 239

### (2-(Pyridin-4-yl)-4-(2,8-diazaspiro[4.5]decan-8-yl)pyrido[3,4-d]pyrimidin-5-yl)methanol (Compound 239) formate

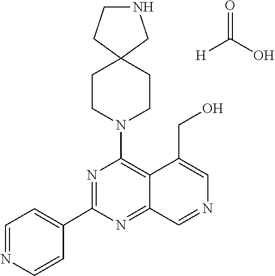 [SMILES: OCc1cncc2nc(-c3ccncc3)nc(N3CCC4(CCNC4)CC3)c12 ; O=CO]

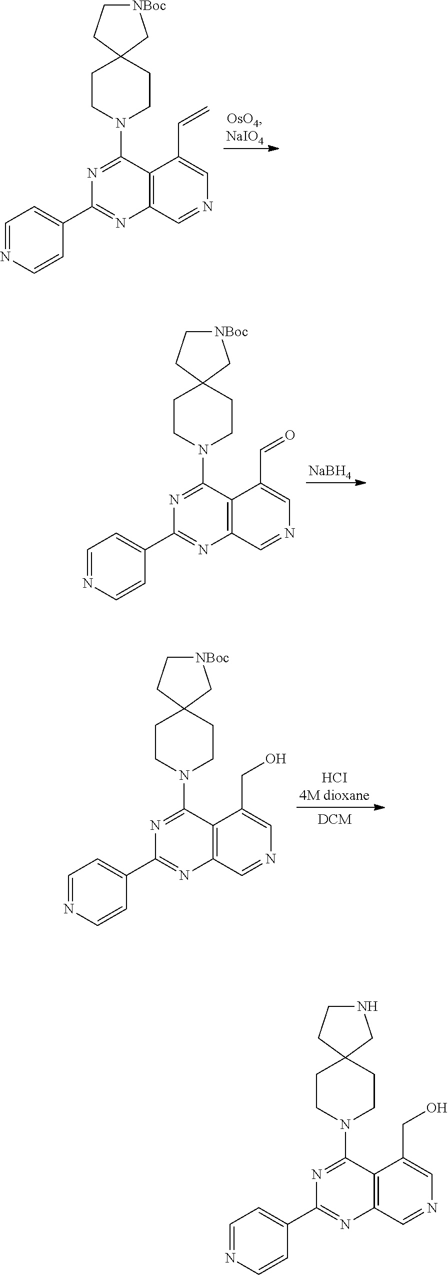 [SMILES: C=Cc1cncc2nc(-c3ccncc3)nc(N3CCC4(CCN(C(=O)OC(C)(C)C)C4)CC3)c12 ; CC(C)(C)OC(=O)N1CCC2(CCN(c3nc(-c4ccncc4)nc4cncc(C=O)c34)CC2)C1 ; "NaBH4" ; CC(C)(C)OC(=O)N1CCC2(CCN(c3nc(-c4ccncc4)nc4cncc(CO)c34)CC2)C1 ; OCc1cncc2nc(-c3ccncc3)nc(N3CCC4(CCNC4)CC3)c12]

### Step 1: tert-Butyl 8-(5-formyl-2-(pyridin-4-yl)pyrido[3,4-d]pyrimidin-4-yl)-2,8-diazaspiro[4.5]decane-2-carboxylate

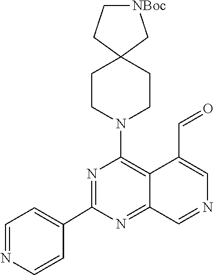 [SMILES: CC(C)(C)OC(=O)N1CCC2(CCN(c3nc(-c4ccncc4)nc4cncc(C=O)c34)CC2)C1]

To a solution of tert-butyl 8-[2-(4-pyridyl)-5-vinyl-pyrido[3,4-d]pyrimidin-4-yl]-2,8-diazaspiro[4.5]decane-2-carboxylate (69 mg, 0.15 mmol) and NMO (34.2 mg, 0.29 mmol) in DCM (2 mL) under an atmosphere of dry nitrogen was added osmium tetroxide (46 uL, 0.01 mmol). The reaction mixture was stirred for 16 hours at room temperature. After complete conversion of olefin to diol, sodium periodate (46.8 mg, 0.22 mmol) in water (1 mL) was added and the mixture was stirred for another 16 hours at room temperature. Reaction mixture was diluted with DCM, washed water (100 mL), brine (100 mL), dried over Na₂SO₄ and evaporated to provide crude tert-butyl 8-[5-formyl-2-(4-pyridyl)pyrido[3,4-d]pyrimidin-4-yl]-2,8-diazaspiro[4.5]decane-2-carboxylate as a brown solid (73 mg). UPLCMS (ESI) m/z: 475.8 [M+H]⁺.

### Step 2: tert-Butyl 8-(5-(hydroxymethyl)-2-(pyridin-4-yl)pyrido[3,4-d]pyrimidin-4-yl)-2,8-diazaspiro[4.5]decane-2-carboxylate

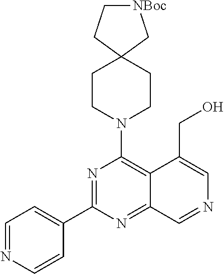 [SMILES: CC(C)(C)OC(=O)N1CCC2(CCN(c3nc(-c4ccncc4)nc4cncc(CO)c34)CC2)C1]

To a solution of tert-butyl 8-[5-formyl-2-(4-pyridyl)pyrido[3,4-d]pyrimidin-4-yl]-2,8-diazaspiro[4.5]decane-2-carboxylate (73 mg, 0.15 mmol) in methanol (2 mL) at 0° C. was added sodium borohydride (5.8 mg, 0.15 mmol) under an atmosphere of dry nitrogen. The reaction mixture was stirred for 6 hours from 0° C. to room temperature. Upon completion of the reduction, a saturated solution of sodium bicarbonate was added to reaction mixture and it was extracted 3 times with ethyl acetate. The organic layers were combined, dried with anhydrous sodium sulfate, filtered and concentrated to dryness. Crude residue was purified by flash chromatography on silica gel (SiO₂, MeOH/DCM) to provide tert-butyl 8-[5-(hydroxymethyl)-2-(4-pyridyl)pyrido[3,4-d]pyrimidin-4-yl]-2,8-diazaspiro[4.5]decane-2-carboxylate as a dark orange gum (25 mg, 0.051 mmol, 34% yield). UPLCMS (ESI) m/z: 477.8 [M+H]⁺.

### Step 3: (2-(Pyridin-4-yl)-4-(2,8-diazaspiro[4.5]decan-8-yl)pyrido[3,4-d]pyrimidin-5-yl)methanol formate

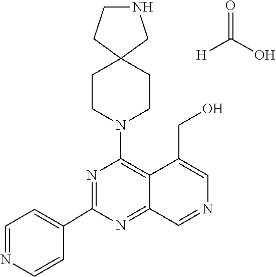 [SMILES: OCc1cncc2nc(-c3ccncc3)nc(N3CCC4(CCNC4)CC3)c12 ; O=CO]

Following the procedure described in Example 199, Step 8 and making non-critical variations as required to replace the substrate with tert-butyl 8-(5-(hydroxymethyl)-2-(pyridin-4-yl)pyrido[3,4-d]pyrimidin-4-yl)-2,8-diazaspiro[4.5]decane-2-carboxylate was obtained the titled compound as a beige solid. ¹H NMR (400 MHz, CD₃OD) δ 9.20 (d, J=3.7 Hz, 1H), 8.75 (d, J=1.7 Hz, 1H), 8.72 (d, J=5.9 Hz, 2H), 8.54 (s, 1H), 8.48 (d, J=6.1 Hz, 2H), 5.13 (s, 2H), 3.89-3.63 (m, 4H), 3.45-3.38 (m, 2H), 3.22-3.01 (m, 2H), 2.13-1.95 (m, 2H), 1.94-1.73 (m, 4H). UPLCMS (ESI) m/z: 377.7 [M+H]⁺.

### Example 240

### 5-Isopropyl-2-(pyridin-4-yl)-4-(2,8-diazaspiro[4.5]decan-8-yl)pyrido[3,4-d]pyrimidine (Compound 240) formate

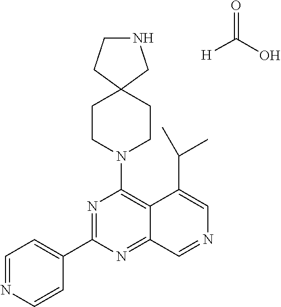 [SMILES: CC(C)c1cncc2nc(-c3ccncc3)nc(N3CCC4(CCNC4)CC3)c12 ; O=CO]

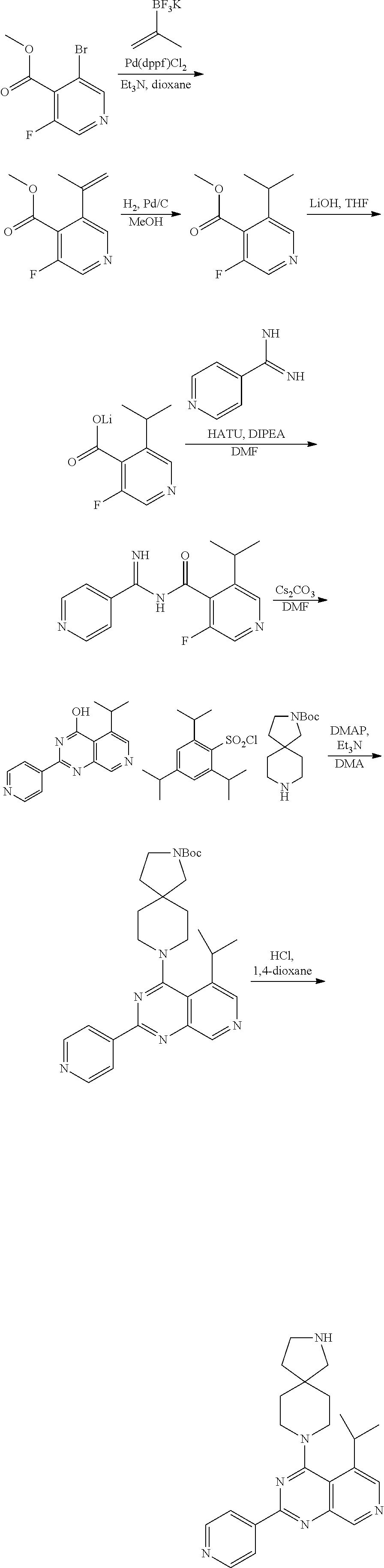

### Step 1: Methyl 3-fluoro-5-(prop-1-en-2-yl)isonicotinate

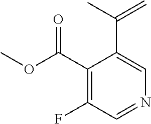

Methyl 3-bromo-5-fluoro-pyridine-4-carboxylate (100 mg, 0.43 mmol) and potassium isopropenyltrifluoroborate (95 mg, 0.64 mmol) were dissolved in 1,4-dioxane (4 mL) and degassed with nitrogen flow for 10 minutes. Triethylamine (0.18 mL, 1.3 mmol) was added while the solution was degasing for another 5 minutes. Then, [1,1′-bis(diphenylphosphino)ferrocene]dichloropalladium(II) (25 mg, 0.03 mmol) was added to the reaction mixture and it was capped under nitrogen and heated to 85° C. for 6 hours. The reaction mixture was cooled to room temperature and it was diluted with a saturated solution of sodium bicarbonate and it was extracted 3 times with EtOAc. Organic layers were combined, dried with anhydrous sodium sulfate, filtered and concentrated to dryness. Crude residue was purified by silica gel flash chromatography (SiO₂, EtOAc/Heptanes) to provide methyl 3-fluoro-5-isopropenyl-pyridine-4-carboxylate as a colorless oil (31 mg, 0.16 mmol, 37% yield). UPLCMS (ESI) m/z: 196.5 [M+H]⁺.

### Step 2: Methyl 3-fluoro-5-isopropylisonicotinate

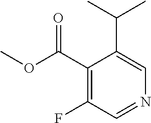

To a solution of methyl 3-fluoro-5-isopropenyl-pyridine-4-carboxylate (31 mg, 0.16 mmol) in methanol (1 mL) under an atmosphere of dry nitrogen was added palladium on carbon (5 mg). The reaction mixture was purged with hydrogen and stirred under 1 atm (ballon) of hydrogen for 1 h at room temperature. After complete conversion the reaction mixture was purged with nitrogen and the reaction mixture was filtered and rinse with methanol. The organic layers were concentrated under reduced pressure to provide methyl 3-fluoro-5-isopropyl-pyridine-4-carboxylate as a yellow solid (30 mg, 0.16 mmol, 97% yield). UPLCMS (ESI) m/z: 198.5 [M+H]⁺.

### Step 3: Lithium 3-fluoro-5-isopropylisonicotinate

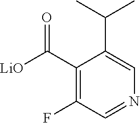

To a solution of methyl 3-fluoro-5-isopropyl-pyridine-4-carboxylate (31 mg, 0.16 mmol) in THF (1 mL) was added 1M solution of lithium hydroxide (310 uL, 0.31 mmol). The reaction mixture was stirred at room temperature for 16h. After complete conversion the reaction mixture was concentrated to dryness to provide lithium 3-fluoro-5-isopropylisonicotinate as a beige solid (30 mg, 0.16 mmol, 102% yield). UPLCMS (ESI) m/z: 184.4 [M+H]⁺.

### Step 4: 5-Isopropyl-2-(pyridin-4-yl)-4-(2,8-diazaspiro[4.5]decan-8-yl)pyrido[3,4-d]pyrimidine formate

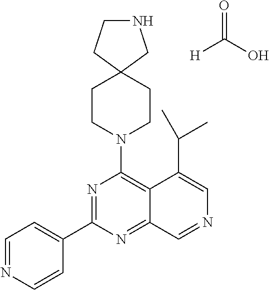 [SMILES: CC(C)c1cncc2nc(-c3ccncc3)nc(N3CCC4(CCNC4)CC3)c12 ; O=CO]

Following the procedure described in Example 225, Steps 1-4 and making non-critical variations as required to replace the carboxylic acid in step 1 with lithium 3-fluoro-5-isopropylisonicotinate was obtained the titled compound as a beige solid. ¹H NMR (400 MHz, DMSO-d₆) δ 9.09 (s, 1H), 8.78 (d, J=5.0 Hz, 2H), 8.69 (s, 1H), 8.37 (s, 1H), 8.33 (d, J=5.0 Hz, 2H), 3.89-3.80 (m, 1H), 3.79-3.69 (m, 2H), 3.68-3.54 (m, 2H), 3.14-3.08 (m, 1H), 3.07-3.02 (m, 1H), 3.01 (br s, 1H), 2.77 (br s, 1H), 1.86 (t, J=7.1 Hz, 1H), 1.77-1.56 (m, 5H), 1.30 (d, J=6.7 Hz, 6H). UPLCMS (ESI) m/z: 389.3 [M+H]⁺.

### Example 241

### 2-(3,5-Dimethyl-1H-pyrazol-4-yl)-4-(2,8-diazaspiro[4.5]decan-8-yl)pyrido[3,4-d]pyrimidine (Compound 241)

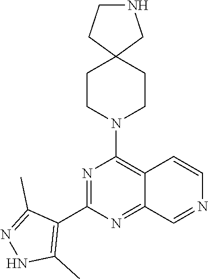 [SMILES: Cc1n[nH]c(C)c1-c1nc(N2CCC3(CCNC3)CC2)c2ccncc2n1]

### Step 1: 2-(3,5-Dimethyl-1H-pyrazol-4-yl)pyrido[3,4-d]pyrimidin-4-ol

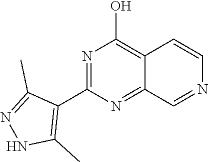 [SMILES: Cc1n[nH]c(C)c1-c1nc(O)c2ccncc2n1]

A solution of 5-amino-3-methyl-1H-pyrazole-4-carboxamide (200 mg, 1.43 mmol), 3-methyl-1H-pyrazole-4-carbaldehyde (157 mg, 1.43 mmol) and copper oxide (11 mg, 0.14 mmol) in DMA (3.1 mL) was stirred at 120° C. rigorously. After 2 days, the reaction mixture was cooled to room temperature and diluted with MeOH (50 mL). This solution was filtered through celite to remove CuO. The filtrate was concentrated under reduced pressure. This residue was triturated with EtOAc and hexane to afford 2-(3,5-Dimethyl-1H-pyrazol-4-yl)pyrido[3,4-d]pyrimidin-4-ol (180 mg, 0.78 mmol, 55% yield) as pale yellow solid.

### Step 2: tert-Butyl 8-(2-(3,5-dimethyl-1H-pyrazol-4-yl)pyrido[3,4-d]pyrimidin-4-yl)-2,8-diazaspiro[4.5]decane-2-carboxylate

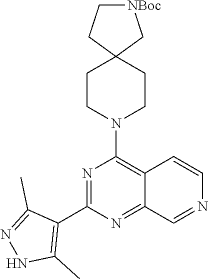 [SMILES: Cc1n[nH]c(C)c1-c1nc(N2CCC3(CCN(C(=O)OC(C)(C)C)C3)CC2)c2ccncc2n1]

A solution of 2-(3,5-dimethyl-1H-pyrazol-4-yl)pyrido[3,4-d]pyrimidin-4-ol (60 mg, 0.25 mmol), N,N-di-iso-propylethylamine (90 μL, 0.50 mmol) and 2,4,6-triisopropylbenzenesulfonyl chloride (79 mg, 0.26 mmol) was stirred at 60° C. for 1 hour. Then, to this mixture was added tert-butyl 2,8-diazaspiro[4.5]decane-2-carboxylate (65 mg, 0.27 mmol). The reaction mixture was allowed to room temperature. After 20 hours, the reaction mixture was poured into water and extracted with EtOAc (3×20 mL). The combined organic layer was washed with brine and dried over MgSO₄ and filtered. The solvent was removed under reduced pressure. The residue was purified by flash column chromatography (SiO₂, Heptanes/EtOAc(with 10% MeOH additive in EtOAc) from 0% to 100%) to afford tert-butyl 8-[2-(3,5-dimethyl-1H-pyrazol-4-yl)pyrido[3,4-d]pyrimidin-4-yl]-2,8-diazaspiro[4.5]decane-2-carboxylate (75 mg, 0.162 mmol, 65% yield) as pale yellow solid. LCMS (ESI) m/z: 464.4 [M+H]⁺.

### Step 3: 2-(3,5-Dimethyl-1H-pyrazol-4-yl)-4-(2,8-diazaspiro[4.5]decan-8-yl)pyrido[3,4-d]pyrimidine

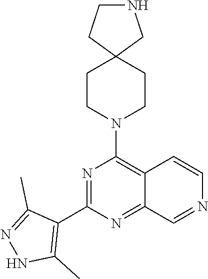 [SMILES: Cc1n[nH]c(C)c1-c1nc(N2CCC3(CCNC3)CC2)c2ccncc2n1]

Following the procedure described in Example 199, Step 8 and making non-critical variations as required to replace the substrate with tert-butyl 8-(2-(3,5-dimethyl-1H-pyrazol-4-yl)pyrido[3,4-d]pyrimidin-4-yl)-2,8-diazaspiro[4.5]decane-2-carboxylate was obtained the titled compound as a pale yellow solid (37 mg, 0.10 mmol, 41% yield). ¹H NMR (400 MHz, DMSO-d₆) 9.05 (s, 1H), 8.42 (d, J=5.6 Hz, 1H), 8.37 (s, 1H), 7.72 (d, J=5.7 Hz, 1H), 3.83-3.75 (m, 4H), 3.18 (t, J=7.2 Hz, 2H), 3.00 (s, 2H), 2.51 (s, 6H), 1.84-1.81 (m, 2H), 1.79-1.61 (m, 4H). LCMS (ESI) m/z: 364.3, [M+H]⁺.

### Example 242

### 8-(methoxymethyl)-2-(pyridin-4-yl)-4-(2,8-diazaspiro[4.5]decan-8-yl)pyrido[3,4-d]pyrimidine (Compound 242)

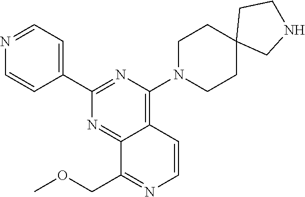 [SMILES: COCc1nccc2c(N3CCC4(CCNC4)CC3)nc(-c3ccncc3)nc12]

### Step 1: tert-butyl 8-(8-chloro-2-(pyridin-4-yl)pyrido[3,4-d]pyrimidin-4-yl)-2,8-diazaspiro[4.5]decane-2-carboxylate

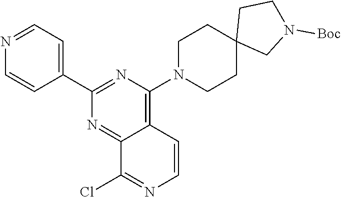 [SMILES: CC(C)(C)OC(=O)N1CCC2(CCN(c3nc(-c4ccncc4)nc4c(Cl)nccc34)CC2)C1]

Following the procedure described in Example 203, step 2 and making non-critical variations as required to replace 8-methoxy-2-(pyridin-4-yl)pyrido[3,4-d]pyrimidin-4-ol with 8-chloro-2-(pyridin-4-yl)pyrido[3,4-d]pyrimidin-4-ol (prepared according to the procedure in WO201452699), the title compound was obtained (8.1 g, 90%) as a yellow solid. LCMS (ESI) m/z: 481.2 [M+H]⁺.

### Step 2: tert-butyl 8-(8-(methoxymethyl)-2-(pyridin-4-yl)pyrido[3,4-d]pyrimidin-4-yl)-2,8-diazaspiro[4.5]decane-2-carboxylate

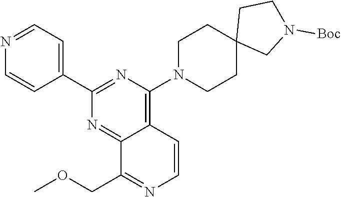 [SMILES: COCc1nccc2c(N3CCC4(CCN(C(=O)OC(C)(C)C)C4)CC3)nc(-c3ccncc3)nc12]

To a solution of tert-butyl 8-(8-chloro-2-(pyridin-4-yl)pyrido[3,4-d]pyrimidin-4-yl)-2,8-diazaspiro[4.5]decane-2-carboxylate (300 mg, 0.62 mmol) and tributyl(methoxymethyl)stannane (313 mg, 0.94 mmol) in DMF (5 mL) was added tetrakis(triphenylphosphine)palladium(0) (72 mg, 0.06 mmol). The reaction mixture was stirred at 130° C. for 16 h under nitrogen atmosphere. After cooling to room temperature, the reaction mixture was diluted with EtOAc (50 mL), washed with sat. aq. KF (30 mL) and brine (30 mL). The organic layer was dried over anhydrous Na₂SO₄, filtered and concentrated in vacuo. The residue was purified by silica gel chromatography (solvent gradient: 0-75% EtOAc in petroleum ether) to give the title compound (27 mg, 9%) as a yellow solid. LCMS (ESI) m/z: 491.3 [M+H]⁺.

### Step 3: 8-(methoxymethyl)-2-(pyridin-4-yl)-4-(2,8-diazaspiro[4.5]decan-8-yl)pyrido[3,4-d]pyrimidine

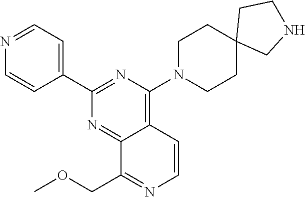 [SMILES: COCc1nccc2c(N3CCC4(CCNC4)CC3)nc(-c3ccncc3)nc12]

Following the procedure described in Example 157, step 7 and making non-critical variations as required to replace tert-butyl 8-(6-benzyl-2-(pyridin-4-yl)pyrido[3,4-d]pyrimidin-4-yl)-2,8-diazaspiro[4.5]decane-2-carboxylate with tert-butyl 8-(8-(methoxymethyl)-2-(pyridin-4-yl)pyrido[3,4-d]pyrimidin-4-yl)-2,8-diazaspiro[4.5]decane-2-carboxylate, the title compound was obtained (15 mg, 18%) as a yellow solid. ¹H NMR (400 MHz, DMSO-d₆) δ 8.77 (d, J=6.0 Hz, 2H), 8.54 (d, J=5.6 Hz, 1H), 8.33 (d, J=6.0 Hz, 2H), 7.81 (d, J=5.6 Hz, 1H), 5.12 (s, 2H), 4.00-3.83 (m, 4H), 3.45 (s, 3H), 2.92-2.84 (m, 2H), 2.70 (s, 2H), 1.75-1.68 (m, 4H), 1.65-1.59 (m, 2H). LCMS (ESI) m/z: 391.2 [M+H].

### Example 243

### 5-(Methoxymethyl)-2-(pyridin-4-yl)-4-(2,8-diazaspiro[4.5]decan-8-yl)pyrido[3,4-d]pyrimidine (Compound 243) formate

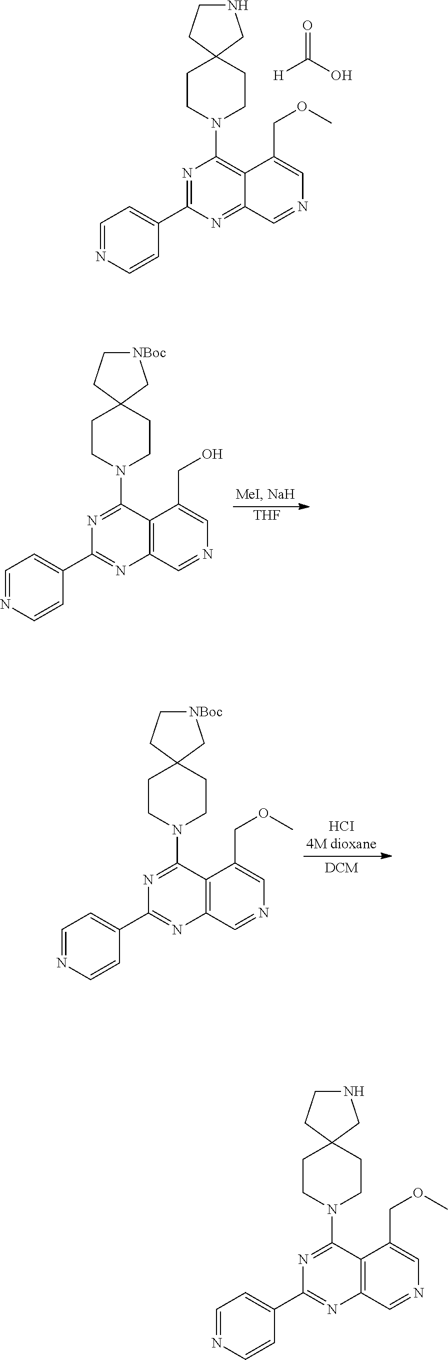

### Step 1: tert-Butyl 8-(5-(methoxymethyl)-2-(pyridin-4-yl)pyrido[3,4-d]pyrimidin-4-yl)-2,8-diazaspiro[4.5]decane-2-carboxylate

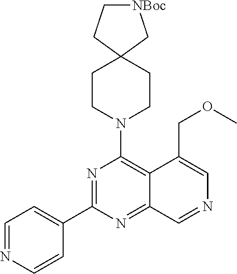 [SMILES: COCc1cncc2nc(-c3ccncc3)nc(N3CCC4(CCN(C(=O)OC(C)(C)C)C4)CC3)c12]

To a solution of tert-butyl 8-[5-(hydroxymethyl)-2-(4-pyridyl)pyrido[3,4-d]pyrimidin-4-yl]-2,8-diazaspiro[4.5]decane-2-carboxylate (48 mg, 0.10 mmol) in THF (1 mL) at 0° C. was added sodium hydride (4.0 mg, 0.10 mmol) under an atmosphere of dry nitrogen. The reaction mixture was stirred for 10 minutes at 0° C. Iodomethane (6.3 uL, 0.10 mmol) was then added and the reaction mixture stirred from 0° C. to room temperature. After complete conversion, a saturated solution of sodium bicarbonate was added to reaction mixture and it was extracted 3 times with ethyl acetate. Organic layers were combined, dried with anhydrous sodium sulfate, filtered and concentrated to dryness. Crude residue was purified by flash chromatography on silica gel (SiO₂, MeOH/DCM) to provide tert-butyl 8-[5-(methoxymethyl)-2-(4-pyridyl)pyrido[3,4-d]pyrimidin-4-yl]-2,8-diazaspiro[4.5]decane-2-carboxylate as a dark solid (18 mg, 0.036 mmol, 36% yield). UPLCMS (ESI) m/z: 491.9 [M+H]⁺.

### Step 2: 5-(Methoxymethyl)-2-(pyridin-4-yl)-4-(2,8-diazaspiro[4.5]decan-8-yl)pyrido[3,4-d]pyrimidine formate

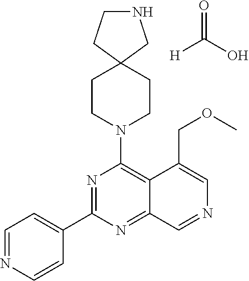

Following the procedure described in Example 199, Step 8 and making non-critical variations as required to replace the substrate with tert-butyl 8-(5-(methoxymethyl)-2-(pyridin-4-yl)pyrido[3,4-d]pyrimidin-4-yl)-2,8-diazaspiro[4.5]decane-2-carboxylate was obtained the titled compound as a beige solid (7.9 mg, 0.017 mmol, 34% yield). ¹H NMR (400 MHz, DMSO-d) δ 9.20 (s, 1H), 8.81-8.76 (m, 2H), 8.65 (s, 1H), 8.35-8.30 (m, 3H), 4.89 (s, 2H), 3.78-3.54 (m, 4H), 3.30 (s, 3H), 3.15-3.00 (m, 2H), 2.98-2.82 (m, 2H), 1.86-1.72 (m, 2H), 1.71-1.59 (m, 4H). UPLCMS (ESI) m/z: 391.8 [M+H]⁺. UPLCMS (ESI) m/z: 391.8 [M+H]⁺.

### Example 244

### 5-(4-(2,8-diazaspiro[4.5]decan-8-yl)pyrido[3,4-d]pyrimidin-2-yl)-4-methylisothiazole (Compound 244) formate

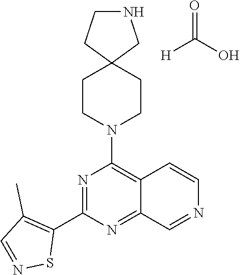

Following the procedure described in Example 241, Steps 1-3 and making non-critical variations as required to replace the aldehyde in step 1 with 4-methylisothiazole-5-carbaldehyde was obtained the titled compound as pale yellow solid. ¹H NMR (400 MHz, CD₃OD) 9.14 (s, 1H), 8.54-8.50 (m, 2H), 8.36 (s, 1H), 7.86 (d, J=5.7 Hz, 1H), 4.10-3.98 (m, 2H), 3.98-3.84 (m, 2H), 3.45 (t, J=7.4 Hz, 2H), 3.24 (s, 2H), 2.74 (s, 3H), 2.09 (t, J=7.4 Hz, 2H), 1.99-1.79 (m, 4H). LCMS (ESI) m/z: 367.2 [M+H]⁺.

### Example 245

### 2-methyl-4-(2-(pyridin-4-yl)-4-(2,8-diazaspiro[4.5]decan-8-yl)pyrido[3,4-d]pyrimidin-8-yl)but-3-yn-2-ol (Compound 245)

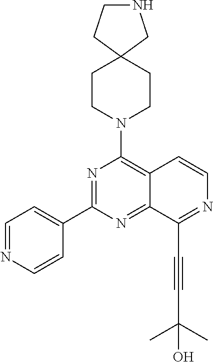 [SMILES: CC(C)(O)C#Cc1nccc2c(N3CCC4(CCNC4)CC3)nc(-c3ccncc3)nc12]

### Step 1: tert-butyl 8-(8-(3-hydroxy-3-methylbut-1-yn-1-yl)-2-(pyridin-4-yl)pyrido[3,4-d]pyrimidin-4-yl)-2,8-diazaspiro[4.5]decane-2-carboxylate

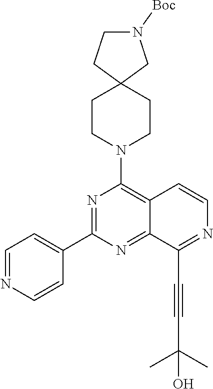 [SMILES: CC(C)(O)C#Cc1nccc2c(N3CCC4(CCN(C(=O)OC(C)(C)C)C4)CC3)nc(-c3ccncc3)nc12]

To a solution of tert-butyl 8-(8-chloro-2-(pyridin-4-yl)pyrido[3,4-d]pyrimidin-4-yl)-2,8-diazaspiro[4.5]decane-2-carboxylate (400 mg, 0.83 mmol) in DMF (8 mL) was added 2-methyl-3-butyn-2-ol (210 mg, 2.49 mmol), triethylamine (1.16 mL, 8.32 mmol), CuI (16 mg, 0.08 mmol), bis(triphenylphosphine)palladium(II) dichloride (58 mg, 0.08 mmol) and triphenylphosphine (44 mg, 0.17 mmol). The mixture was heated to 60° C. for 8 h under nitrogen atmosphere. After cooling to room temperature, the mixture was diluted with EtOAc (100 mL), washed with water (50 mL×3) and brine (50 mL), dried over anhydrous Na₂SO₄, filtered and concentrated in vacuo. The crude residue was purified by silica gel chromatography (solvent gradient: 0-3% MeOH in DCM) to give the title compound (220 mg, 50%) as a yellow solid. LCMS (ESI) m/z: 529.3 [M+H]⁺.

### Step 2: 2-methyl-4-(2-(pyridin-4-yl)-4-(2,8-diazaspiro[4.5]decan-8-yl)pyrido[3,4-d]pyrimidin-8-yl)but-3-yn-2-ol

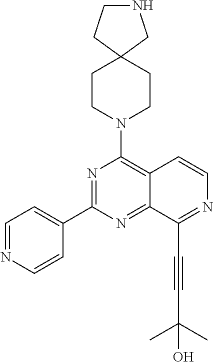 [SMILES: CC(C)(O)C#Cc1nccc2c(N3CCC4(CCNC4)CC3)nc(-c3ccncc3)nc12]

A solution of tert-butyl 8-(8-(3-hydroxy-3-methylbut-1-yn-1-yl)-2-(pyridin-4-yl)pyrido[3,4-d]pyrimidin-4-yl)-2,8-diazaspiro[4.5]decane-2-carboxylate (220 mg, 0.42 mmol) in 5% trifluoroacetic acid in hexafluoroisopropanol (4 mL) was stirred at room temperature for 0.5 h. The mixture was quenched with solid NaHCO₃ (2 g) and the mixture was stirred for 10 min, diluted with MeOH (10 mL), filtered and the filtrate was concentrated in vacuo to give the title compound (170 mg, crude) as yellow oil. The crude residue (70 mg) was purified by reverse phase chromatography (acetonitrile 23-53%/0.05% NH₃·H₂O+10 mM NH₄HCO₃ in water) to give the title compound (2 mg, 2%) as a yellow solid. ¹H NMR (400 MHz, DMSO-d₆) δ 8.84-8.71 (m, 2H), 8.52 (d, J=5.6 Hz, 1H), 8.42-8.32 (m, 2H), 7.83 (d, J=5.6 Hz, 1H), 5.73 (br s, 1H), 4.02-3.89 (m, 4H), 3.15 (t, J=7.2 Hz, 2H), 2.96 (s, 2H), 1.82 (t, J=7.2 Hz, 2H), 1.80-1.72 (m, 4H), 1.62 (s, 6H). One of exchangeable protons not observed. LCMS (ESI) m/z: 429.2 [M+H]⁺.

### Example 246

### 2-(pyridin-4-yl)-4-(2,8-diazaspiro[4.5]decan-8-yl)-5-(trifluoromethyl)pyrido[3,4-d]pyrimidine (Compound 246)

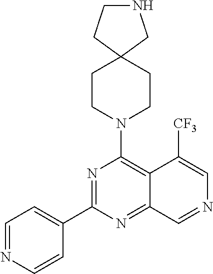 [SMILES: FC(F)(F)c1cncc2nc(-c3ccncc3)nc(N3CCC4(CCNC4)CC3)c12]

Following the procedure described in Example 225, Steps 1-4 and making non-critical variations as required to replace the acid in step 1 with 3-fluoro-5-(trifluoromethyl)isonicotinic acid was obtained the titled compound as pale yellow solid. ¹H NMR (400 MHz, DMSO-d₆) δ 9.44 (s, 1H), 8.94 (s, 1H), 8.78 (br s, 2H), 8.30 (d, J=4.9 Hz, 2H), 3.99-3.77 (m, 3H), 3.68-3.50 (m, 3H), 3.35-3.12 (m, 2H), 2.01-1.80 (m, 1H), 1.72-1.42 (m, 5H). UPLCMS (ESI) m/z: 415.3 [M+H].

### Example 247

### 2-methyl-4-(4-(2-methyl-2,8-diazaspiro[4.5]decan-8-yl)-2-(pyridin-4-yl)pyrido[3,4-d]pyrimidin-8-yl)but-3-yn-2-ol (Compound 247)

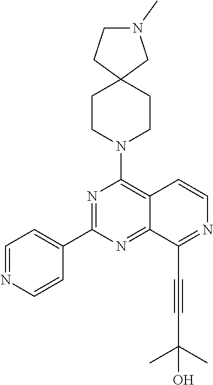 [SMILES: CN1CCC2(CCN(c3nc(-c4ccncc4)nc4c(C#CC(C)(C)O)nccc34)CC2)C1]

Following the procedure described in Example 102 and making non-critical variations as required to replace 4-(2,8-diazaspiro[4.5]decan-8-yl)-2-(4-pyridyl)pyrido[3,4-d]pyrimidine hydrochloride with 2-methyl-4-(2-(pyridin-4-yl)-4-(2,8-diazaspiro[4.5]decan-8-yl)pyrido[3,4-d]pyrimidin-8-yl)but-3-yn-2-ol trifluoroacetate, the title compound was obtained as a yellow solid. ¹H NMR (400 MHz, DMSO-d₆) δ 8.84-8.72 (m, 2H), 8.50 (d, J=5.6 Hz, 1H), 8.42-8.32 (m, 2H), 7.81 (d, J=5.6 Hz, 1H), 5.73 (s, 1H), 4.02-3.92 (m, 2H), 3.91-3.80 (m, 2H), 2.53-2.51 (m, 2H), 2.38 (s, 2H), 2.23 (s, 3H), 1.80-1.66 (m, 6H), 1.62 (s, 6H). LCMS (ESI) m/z: 443.2 [M+H]⁺

### Example 248

### 5-isopropoxy-2-(pyridin-4-yl)-4-(2,8-diazaspiro[4.5]decan-8-yl)pyrido[3,4-d]pyrimidine (Compound 248) formate

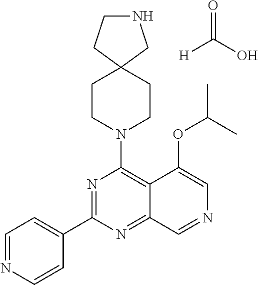

Following the procedure described in Example 233, Steps 3-5 and making non-critical variations as required to replace the methoxide with isopropoxide was obtained the titled compound as pale yellow solid. ¹H NMR (400 MHz, DMSO-d₆) δ 8.77 (s, 1H), 8.74 (dd, J=4.5, 1.5 Hz, 2H), 8.37 (s, 1H), 8.32 (s, 1H), 8.27 (dd, J=4.5, 1.5 Hz, 2H), 4.98-4.88 (m, 1H), 3.81-3.66 (m, 4H), 3.09 (t, J=7.1 Hz, 2H), 2.91 (s, 2H), 1.75 (t, J=7.2 Hz, 2H), 1.70-1.59 (m, 4H), 1.38 (d, J=6.0 Hz, 6H). UPLCMS (ESI) m/z: 405.3 [M+H]⁺.

### Example 249

### 2-(Pyridin-4-yl)-4-(2,8-diazaspiro[4.5]decan-8-yl)pyrido[3,4-d]pyrimidin-5-ol (Compound 249) formate

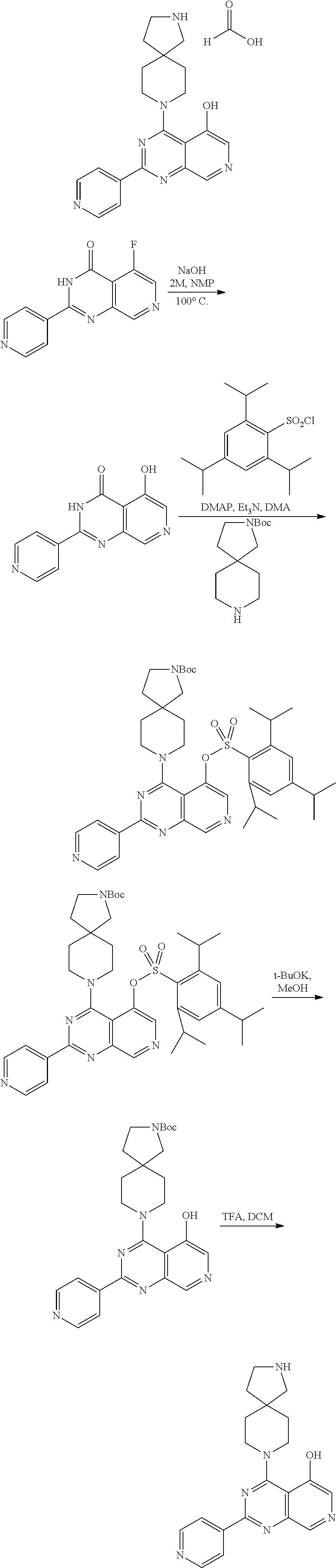

### Step 1: 2-(Pyridin-4-yl)pyrido[3,4-d]pyrimidine-4,5-diol

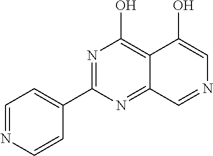 [SMILES: Oc1cncc2nc(-c3ccncc3)nc(O)c12]

To a solution of 5-fluoro-2-(4-pyridyl)-3H-pyrido[3,4-d]pyrimidin-4-one (264 mg, 1.1 mmol) in NMP (4 mL) was added a 2 M solution of sodium hydroxide (2 mL, 4 mmol). The reaction mixture was capped and stirred at 110° C. for 16 hours. The reaction mixture was concentrated with a stream of air and the crude residue used directly for next step. Provided 5-hydroxy-2-(pyridin-4-yl)pyrido[3,4-d]pyrimidin-4 (3H)-one as a brown solid (261 mg, 1.1 mmol, 99% yield). UPLCMS (ESI) m/z: 241.4 [M+H]⁺.

### Step 2: tert-Butyl 8-(2-(pyridin-4-yl)-5-(((2,4,6-triisopropylphenyl)sulfonyl)oxy)pyrido[3,4-d]pyrimidin-4-yl)-2,8-diazaspiro[4.5]decane-2-carboxylate

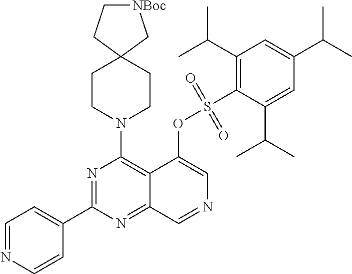 [SMILES: CC(C)c1cc(C(C)C)c(S(=O)(=O)Oc2cncc3nc(-c4ccncc4)nc(N4CCC5(CCN(C(=O)OC(C)(C)C)C5)CC4)c23)c(C(C)C)c1]

To a solution of 5-hydroxy-2-(4-pyridyl)-3H-pyrido[3,4-d]pyrimidin-4-one (262 mg, 1.1 mmol), 4-dimethylaminopyridine (13 mg, 0.11 mmol) and N,N-diisopropylethylamine (570 uL, 3.3 mmol) in DMA (5.5 mL) was added 2,4,6-triisopropylbenzenesulfonyl chloride (726 mg, 2.4 mmol) and the reaction mixture was stirred at room temperature for 60 minutes. tert-butyl 2,8-diazaspiro[4.5]decane-2-carboxylate (314 mg, 1.3 mmol) was added and the mixture was stirred at room temperature for 16 hours. A saturated solution of NH₄Cl (25 mL) and 2-methyltetrahydrofuran (40 mL) were added. The layers were separated and the organic layer was washed with water (30 mL), brine (30 mL), dried over Na₂SO₄, filtered and concentrated to a brown oil. The crude oil was purified by flash chromatography on silica (SiO₂, (EtOAc/MeOH; 3:1)/heptanes) to provide tert-butyl 8-[2-(4-pyridyl)-5-(2,4,6-triisopropylphenyl)sulfonyloxy-pyrido[3,4-d]pyrimidin-4-yl]-2,8-diazaspiro[4.5]decane-2-carboxylate as an orange gum (94 mg, 0.132 mmol, 12% yield). UPLCMS (ESI) m/z: 730.1 [M+H]⁺.

### Step 3: tert-Butyl 8-(5-hydroxy-2-(pyridin-4-yl)pyrido[3,4-d]pyrimidin-4-yl)-2,8-diazaspiro[4.5]decane-2-carboxylate

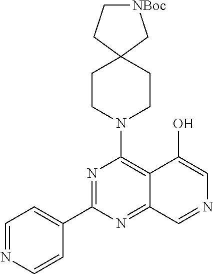 [SMILES: CC(C)(C)OC(=O)N1CCC2(CCN(c3nc(-c4ccncc4)nc4cncc(O)c34)CC2)C1]

Potassium tert-butoxide (18 mg, 0.16 mmol) was added to a stirring solution of tert-butyl 8-[2-(4-pyridyl)-5-(2,4,6-triisopropylphenyl)sulfonyloxy-pyrido[3,4-d]pyrimidin-4-yl]-2,8-diazaspiro[4.5]decane-2-carboxylate (57 mg, 0.08 mmol) in methanol (0.52 mL). The reaction mixture was stirred at room temperature for 16 hours. The resulting solution was concentrated under reduced pressure to provide tert-butyl 8-(5-hydroxy-2-(pyridin-4-yl)pyrido[3,4-d]pyrimidin-4-yl)-2,8-diazaspiro[4.5]decane-2-carboxylate as a bright orange oil (36 mg, 0.080 mmol, 100% yield). UPLCMS (ESI) m/z: 463.8 [M+H].

### Step 4: 2-(Pyridin-4-yl)-4-(2,8-diazaspiro[4.5]decan-8-yl)pyrido[3,4-d]pyrimidin-5-ol formate

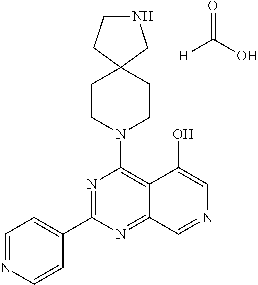

Trifluoroacetic acid (0.25 mL, 3.2 mmol) was added to a stirring solution of tert-butyl 8-[5-hydroxy-2-(4-pyridyl)pyrido[3,4-d]pyrimidin-4-yl]-2,8-diazaspiro[4.5]decane-2-carboxylate (36 mg, 0.08 mmol) in DCM (0.4 mL) at 21° C. The resulting solution was allowed stirred for 3 hours. The resulting solution was concentrated under reduced pressure and azeotroped 3 times to yield a yellow oil. The crude material was purified by reverse phase flash chromatography on C18 (MeCN in water-10 mM ammonium formate pH=3.8). Pure fractions were directly lyophilized to provide 2-(pyridin-4-yl)-4-(2,8-diazaspiro[4.5]decan-8-yl)pyrido[3,4-d]pyrimidin-5-ol, formate salt as a light brown solid (7.8 mg, 0.020 mmol, 25% yield). ¹H NMR (400 MHz, DMSO-d₆) δ 8.73 (dd, J=4.5, 1.5 Hz, 2H), 8.38 (s, 1H), 8.32 (s, 1H), 8.28 (dd, J=4.5, 1.6 Hz, 2H), 8.01 (s, 1H), 3.85-3.65 (m, 4H), 3.21-3.14 (m, 2H), 2.98 (s, 2H), 1.82 (t, J=7.3 Hz, 2H), 1.78-1.59 (m, 4H). UPLCMS (ESI) m/z: 363.3 [M+H]⁺.

### Example 250

### 1-(8-(5-methoxy-2-(pyridin-4-yl)pyrido[3,4-d]pyrimidin-4-yl)-2,8-diazaspiro[4.5]decan-2-yl)-2-methylpropan-2-ol (Compound 250)

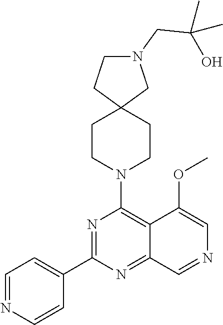 [SMILES: COc1cncc2nc(-c3ccncc3)nc(N3CCC4(CCN(CC(C)(C)O)C4)CC3)c12]

### Step 1: 3,5-difluoro-N-(imino(pyridin-4-yl)methyl)isonicotinamide

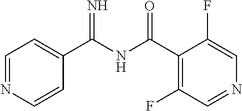 [SMILES: N=C(NC(=O)c1c(F)cncc1F)c1ccncc1]

To a solution of 3,5-difluoroisonicotinic acid (175 g, 1.1 mol) in DMF (2.1 L) was added HATU (460 g, 1.21 mol) and N,N-diisopropylethylamine (545.4 mL, 3.3 mol) at room temperature. After stirring for 5 min, isonicotinimidamide hydrochloride (182 g, 1.16 mol) was added to this reaction mixture. The resulting mixture was stirred for 5 h at room temperature. The reaction mixture was added dropwise to water (4.2 L) and stirred for 30 min. A white precipitate was formed and filtered off, the filter cake was washed with water (500 mL×2), petroleum ether (500 mL×2) and dried in vacuo to give the title compound (124 g, 43%) as a white solid. LCMS (ESI) m/z: 263.1 [M+H]⁺.

### Step 2: 5-fluoro-2-(pyridin-4-yl)pyrido[3,4-d]pyrimidin-4-ol

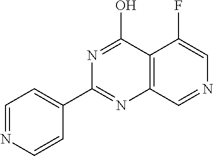 [SMILES: Oc1nc(-c2ccncc2)nc2cncc(F)c12]

To a solution of 3,5-difluoro-N-(imino(pyridin-4-yl)methyl)isonicotinamide (124 g, 472.9 mmol) in DMF (900 mL) was added Cs₂CO₃ (185 g, 567.5 mmol). The mixture was stirred at 100° C. for 3 h. After cooling to room temperature, the reaction mixture was added to water (1.8 L) and stirred for 30 min. The mixture was adjusted to pH 5 with AcOH, then stirred for 30 min. A white precipitate was formed and filtered off, the filter cake was washed with water (400 mL×2), petroleum ether (400 mL×2) and dried in vacuo to give the title compound (91 g, 80%) as a white solid. LCMS (ESI) m/z: 242.6 [M+H]⁺.

### Step 3: 5-methoxy-2-(pyridin-4-yl)pyrido[3,4-d]pyrimidin-4-ol

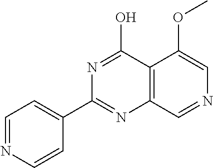 [SMILES: COc1cncc2nc(-c3ccncc3)nc(O)c12]

To a solution of 5-fluoro-2-(pyridin-4-yl)pyrido[3,4-d]pyrimidin-4-ol (91 g, 375.7 mmol) in DMF (600 mL) was added sodium methoxide (60.9 g, 1.13 mol). The mixture was stirred at 40° C. for 3 h. After cooling to room temperature, the reaction mixture was added to water (1.2 L) and stirred for 30 min. The mixture was adjusted to pH 5 with AcOH, then stirred for 30 min. A white precipitate was formed and filtered off, the filter cake was washed with water (350 mL×2), petroleum ether (350 mL×2) and dried in vacuo to give the title compound (90 g, 94%) as a white solid. LCMS (ESI) m/z: 254.7 [M+H]⁺.

### Step 4: tert-butyl 8-(5-methoxy-2-(pyridin-4-yl)pyrido[3,4-d]pyrimidin-4-yl)-2,8-diazaspiro[4.5]decane-2-carboxylate

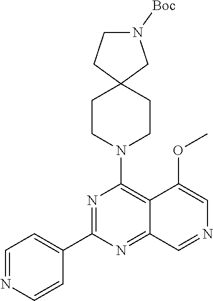 [SMILES: COc1cncc2nc(-c3ccncc3)nc(N3CCC4(CCN(C(=O)OC(C)(C)C)C4)CC3)c12]

A mixture of 5-methoxy-2-(pyridin-4-yl)pyrido[3,4-d]pyrimidin-4-ol (90 g, 354 mmol), PyBOP (221 g, 425 mmol) and triethylamine (148 mL, 1.06 mol) in DMF (900 mL) was stirred at room temperature for 10 min before tert-butyl 2,8-diazaspiro[4.5]decane-2-carboxylate (85.1 g, 354 mmol) was added. The reaction was stirred at room temperature for 16 h. The reaction mixture was quenched with water (1.8 L), extracted with EtOAc (2.5 L×3). The combined organic layers were washed with water (2 L×3) and brine (2 L), dried over anhydrous Na₂SO₄, filtered and concentrated in vacuo. The crude residue was purified by silica gel chromatography (solvent gradient: 0-3% MeOH in DCM) to give the title compound (141 g, 84%) as a yellow solid. LCMS (ESI) m/z: 477.2 [M+H]⁺.

### Step 5: 5-methoxy-2-(pyridin-4-yl)-4-(2,8-diazaspiro[4.5]decan-8-yl)pyrido[3,4-d]pyrimidine hydrochloride

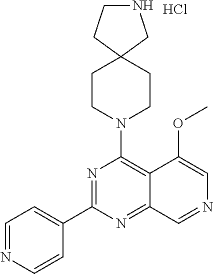 [SMILES: COc1cncc2nc(-c3ccncc3)nc(N3CCC4(CCNC4)CC3)c12 ; Cl]

To a solution of tert-butyl 8-(5-methoxy-2-(pyridin-4-yl)pyrido[3,4-d]pyrimidin-4-yl)-2,8-diazaspiro[4.5]decane-2-carboxylate (80 g, 167.9 mmol) in dioxane (300 mL) was added 4M HCl in dioxane (300 mL, 1.2 mol). The mixture was stirred at room temperature for 2 h. The mixture was concentrated in vacuo to give the title compound (63 g, crude) as a yellow solid that required no further purification. LCMS (ESI) m/z: 376.9 [M+H]⁺.

### Step 6: 1-(8-(5-methoxy-2-(pyridin-4-yl)pyrido[3,4-d]pyrimidin-4-yl)-2,8-diazaspiro[4.5]decan-2-yl)-2-methylpropan-2-ol

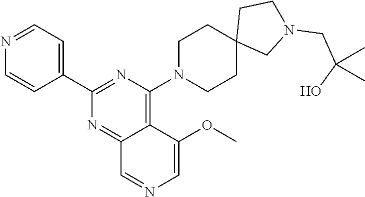 [SMILES: COc1cncc2nc(-c3ccncc3)nc(N3CCC4(CCN(CC(C)(C)O)C4)CC3)c12]

To a solution of 5-methoxy-2-(pyridin-4-yl)-4-(2,8-diazaspiro[4.5]decan-8-yl)pyrido[3,4-d]pyrimidine hydrochloride (34.6 g, 83.8 mmol) in EtOH (300 mL) was added isobutyleneoxide (20.8 mL, 251.4 mmol) and triethylamine (58.4 mL, 419 mmol). The mixture was stirred at 80° C. for 16 h. After cooling to room temperature, the mixture was concentrated in vacuo. The crude residue was purified by silica gel chromatography (solvent gradient: 0-10% MeOH in DCM) to give a crude product (61 g). The crude product was purified by reverse phase chromatography (acetonitrile 2-32%/0.225% formic acid in water) to give a formate product (36 g, formate). The formate product was dissolved in MeOH (100 mL) and the mixture was adjusted to pH 9 with NH₃·H₂O, then was purified by reverse phase chromatography (acetonitrile 30-70%/0.05% NH₃·H₂O+10 mM NH₄HCO₃ in water) to give the title compound (29 g, 42%). ¹H NMR (400 MHz, DMSO-d₆) δ 8.80 (s, 1H), 8.76-8.72 (m, 2H), 8.31 (s, 1H), 8.29-8.26 (m, 2H), 4.06 (s, 3H), 4.03 (s, 1H), 3.72-3.57 (m, 4H), 2.67 (t, J=6.9 Hz, 2H), 2.53 (s, 2H), 2.31 (s, 2H), 1.70-1.59 (m, 6H), 1.08 (s, 6H). LCMS (ESI) m/z: 449.0 [M+H]⁺.

### Example 251

### 4-(4-(2,8-diazaspiro[4.5]decan-8-yl)pyrido[3,4-d]pyrimidin-2-yl)pyridine 1-oxide (Compound 251)

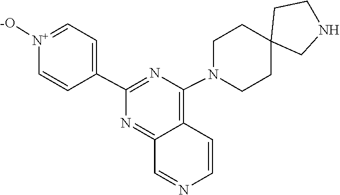

### Step 1: tert-butyl 8-(2-(tributylstannyl)pyrido[3,4-d]pyrimidin-4-yl)-2,8-diazaspiro[4.5]decane-2-carboxylate

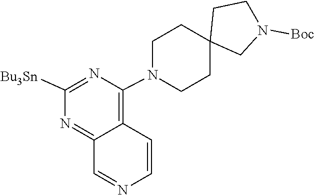

To a solution of 1,1,1,2,2,2-hexabutyldistannane (4.64 g, 8.0 mmol), tert-butyl 8-(2-chloropyrido[3,4-d]pyrimidin-4-yl)-2,8-diazaspiro[4.5]decane-2-carboxylate (0.8 g, 1.98 mmol) in dioxane (5 mL) was added Pd(t-Bu₃P)₂ (101 mg, 0.20 mmol). The mixture was heated to 100° C. for 16 h under nitrogen atmosphere. After cooling to room temperature, the reaction was concentrated in vacuo. The crude residue was purified by silica gel chromatography (solvent gradient: 0-30% EtOAc in petroleum ether) to give the title compound (400 mg, 30%) as colorless oil. LCMS (ESI) m/z: 660.2 [M+H]⁺.

### Step 2: 4-(4-(2-(tert-butoxycarbonyl)-2,8-diazaspiro[4.5]decan-8-yl)pyrido[3,4-d]pyrimidin-2-yl)pyridine 1-oxide

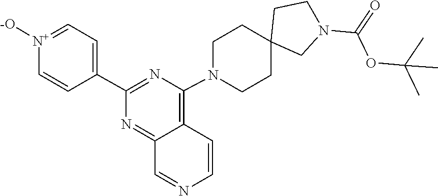

To a solution of 4-bromopyridine 1-oxide (270 mg, 1.55 mmol) and tert-butyl 8-(2-(tributylstannyl)pyrido[3,4-d]pyrimidin-4-yl)-2,8-diazaspiro[4.5]decane-2-carboxylate (450 mg, 0.68 mmol) in dioxane (10 mL) was added Pd(t-Bu₃P)₂ (35 mg, 0.07 mmol). The mixture was heated to 70° C. for 16 h under nitrogen atmosphere. After cooling to room temperature, the reaction was concentrated in vacuo. The crude residue was purified by prep-TLC (DCM/MeOH=10:1) to give the title compound (140 mg, 44%) as yellow oil. LCMS (ESI) m/z: 463.3 [M+H]⁺.

### Step 3: 4-(4-(2,8-diazaspiro[4.5]decan-8-yl)pyrido[3,4-d]pyrimidin-2-yl)pyridine 1-oxide

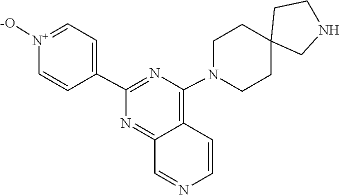

Following the procedure described in Example 157, step 7 and making non-critical variations as required to replace tert-butyl 8-(6-benzyl-2-(pyridin-4-yl)pyrido[3,4-d]pyrimidin-4-yl)-2,8-diazaspiro[4.5]decane-2-carboxylate with 4-(4-(2-(tert-butoxycarbonyl)-2,8-diazaspiro[4.5]decan-8-yl)pyrido[3,4-d]pyrimidin-2-yl)pyridine 1-oxide, the title compound was obtained (46 mg, 45%) as a yellow solid. ¹H NMR (400 MHz, DMSO-d₆) δ 9.22 (s, 1H), 8.58 (d, J=5.6 Hz, 1H), 8.38 (s, 1H), 8.36-8.32 (m, 4H), 7.87 (d, J=5.6 Hz, 1H), 4.02-4.89 (m, 4H), 3.10 (t, J=7.2 Hz, 2H), 3.01 (s, 2H), 1.85 (t, J=7.2 Hz, 2H), 1.80-1.70 (m, 4H). LCMS (ESI) m/z: 363.3 [M+H].

### Example 252

### 5-Methoxy-4-(2-methyl-2,8-diazaspiro[4.5]decan-8-yl)-2-(pyridin-4-yl)pyrido[3,4-d]pyrimidine formate (Compound 252)

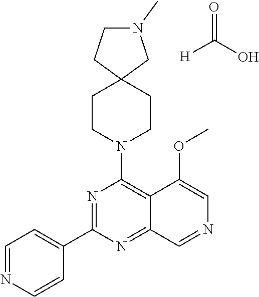

To a solution 5-methoxy-2-(pyridin-4-yl)-4-(2,8-diazaspiro[4.5]decan-8-yl)pyrido[3,4-d]pyrimidine (48.5 mg, 0.13 mmol) and paraformaldehyde (0.01 mL, 0.19 mmol) were in DCM (6.4 mL) was added acetic acid (7 uL, 0.13 mmol) and sodium triacetoxyborohydride (82 mg, 0.39 mmol). The reaction mixture was stirred at room temperature for 48 hours. The reaction mixture was dissolved with MeOH and concentrated under reduced pressure. The crude material was purified by reverse phase column chromatography over C18 (MeCN/Ammonium formate pH 3.8 buffer) to afford 5-methoxy-4-(2-methyl-2,8-diazaspiro[4.5]decan-8-yl)-2-(4-pyridyl)pyrido[3,4-d]pyrimidine (20 mg, 0.053 mmol, 41% yield) as a beige solid. ¹H NMR (400 MHz, DMSO-d₆) δ 8.79 (s, 1H), 8.71 (dd, J=4.6, 1.5 Hz, 2H), 8.32 (s, 1H), 8.29 (dd, J=4.6, 1.5 Hz, 2H), 8.26 (s, 1H), 4.03 (s, 3H), 3.76-3.67 (m, 2H), 3.67-3.57 (m, 2H), 3.23-3.12 (m, 2H), 3.07-2.95 (m, 2H), 2.68 (s, 3H), 1.94-1.87 (m, 2H), 1.79-1.66 (m, 4H). LCMS (ESI) m/z: 391.8 [M+H]⁺.

### Example 253

### 1-((8-(5-methoxy-2-(pyridin-4-yl)pyrido[3,4-d]pyrimidin-4-yl)-2,8-diazaspiro[4.5]decan-2-yl)methyl)cyclobutan-1-ol (Compound 253) formate

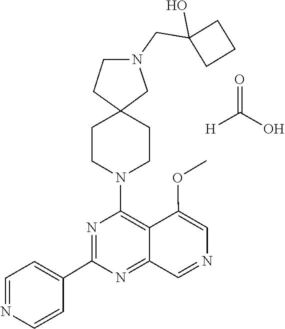

A solution of 5-methoxy-2-(pyridin-4-yl)-4-(2,8-diazaspiro[4.5]decan-8-yl)pyrido[3,4-d]pyrimidine, pentahydrochloride (75 mg, 0.130 mmol), 1-oxaspiro[2.3]hexane (16.9 mg, 0.200 mmol), N,N-di-iso-propylethylamine (0.14 mL, 0.8100 mmol) in methanol (1.50 mL) was stirred at 65° C. After 20 hours, the reaction mixture was concentrated under reduced pressure. The residue was purified by reverse phase column chromatography over C18 (MeCN/Ammonium formate pH 3.8 buffer) to afford 1-[[8-[5-methoxy-2-(4-pyridyl)pyrido[3,4-d]pyrimidin-4-yl]-2,8-diazaspiro[4.5]decan-2-yl]methyl]cyclobutanol, formate salt (6.2 mg, 0.0135 mmol, 10% yield) as white powder. ¹H NMR (500 MHz, CD₃OD) 8.86 (s, 1H), 8.72 (d, J=5.0 Hz, 2H), 8.54 (s, 1H), 8.45 (dd, J=5.0, 1H), 8.28 (s, 1H), 4.16 (s, 3H), 3.97-3.84 (m, 2H), 3.78-3.74 (m, 2H), 3.51 (s, 2H), 3.36 (s, 2H), 3.35-3.34 (m, 2H), 2.31-2.16 (m, 4H), 2.11 (t, J=7.0 Hz, 2H), 1.99-1.83 (m, 5H), 1.77-1.59 (m, 1H). LCMS (ESI) m/z: 461.2, [M+H]⁺.

### Example 254

### 5-(allyloxy)-2-(pyridin-4-yl)-4-(2,8-diazaspiro[4.5]decan-8-yl)pyrido[3,4-d]pyrimidine (Compound 254)

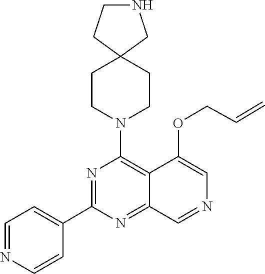 [SMILES: C=CCOc1cncc2nc(-c3ccncc3)nc(N3CCC4(CCNC4)CC3)c12]

Following the procedure described in Example 233, Steps 3-5 and making non-critical variations as required to replace the methoxide with allyloxide was obtained the titled compound as pale yellow solid. ¹H NMR (400 MHz, DMSO-d₆) δ 8.83 (s, 1H), 8.77 (dd, J=4.5, 1.5 Hz, 2H), 8.35 (s, 1H), 8.33 (s, 1H), 8.30 (dd, J=4.5, 1.5 Hz, 2H), 6.17 (ddt, J=16.0, 10.8, 5.5 Hz, 1H), 5.50 (dd, J=17.2, 1.5 Hz, 1H), 5.38 (d, J=10.5 Hz, 1H), 4.90 (d, J=5.5 Hz, 2H), 3.81-3.65 (m, 4H), 3.07 (t, J=7.1 Hz, 2H), 2.87 (s, 2H), 1.74 (t, J=7.3 Hz, 2H), 1.69-1.59 (m, 4H). UPLCMS (ESI) m/z: 403.3 [M+H]⁺.

### Example 255

### 5-Methoxy-4-(2-(oxetan-3-ylmethyl)-2,8-diazaspiro[4.5]decan-8-yl)-2-(pyridin-4-yl)pyrido[3,4-d]pyrimidine (Compound 255)

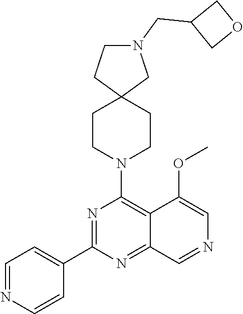 [SMILES: COc1cncc2nc(-c3ccncc3)nc(N3CCC4(CCN(CC5COC5)C4)CC3)c12]

Following the procedure described in Example 252 and making non-critical variations as required to replace the paraformaldehyde with oxetane-3-carbaldehyde was obtained the titled compound as pale yellow solid (8.6 mg, 0.019 mmol, 14% yield). ¹H NMR (400 MHz, DMSO-d₆) 8.80 (s, 1H), 8.74 (d, J=5.1 Hz, 2H), 8.31 (s, 1H), 8.27 (d, J=5.9 Hz, 2H), 4.61 (dd, J=7.7, 5.9 Hz, 2H), 4.24 (t, J=6.1 Hz, 2H), 4.05 (s, 3H), 3.76-3.66 (m, 3H), 3.61-3.60 (m, 3H), 3.09 (dt, J=14.0, 6.9 Hz, 1H), 2.66 (d, J=7.4 Hz, 2H), 2.36 (s, 2H), 1.75-1.49 (m, 6H). LCMS (ESI) m/z: 447.3, [M+H].

### Example 256

### 4-(2-(but-3-yn-1-yl)-2,8-diazaspiro[4.5]decan-8-yl)-2-(pyridin-4-yl)pyrido[3,4-d]pyrimidine (Compound 256)

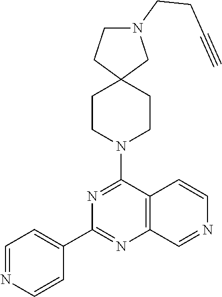 [SMILES: C#CCCN1CCC2(CCN(c3nc(-c4ccncc4)nc4cnccc34)CC2)C1]

To a solution of 4-(2,8-diazaspiro[4.5]decan-8-yl)-2-(4-pyridyl)pyrido[3,4-d]pyrimidine hydrochloride (100 mg, 0.26 mmol) in EtOH (2 mL) was added N,N-diisopropylethylamine (0.27 mL, 1.57 mmol) and 4-bromobut-1-yne (0.2 mL, 2.09 mmol). The mixture was stirred at room temperature for 16 h. The resulting mixture was directly purified by reverse phase chromatography with no further workup (acetonitrile 30-60%/0.05% NH₃·H₂O+10 mM NH₄HCO₃ in water) to give the title compound (2.3 mg, 2%) as a white solid. ¹H NMR (400 MHz, DMSO-d₆) δ 9.26 (s, 1H), 8.81-8.73 (m, 2H), 8.59 (d, J=5.6 Hz, 1H), 8.36-8.28 (m, 2H), 7.89 (d, J=5.6 Hz, 1H), 4.03-3.85 (m, 4H), 2.79 (t, J=2.8 Hz, 1H), 2.62-2.57 (m, 2H), 2.53-2.51 (m, 2H), 2.48 (s, 2H), 2.36-2.29 (m, 2H), 1.81-1.67 (m, 6H). LCMS (ESI) m/z: 399.2 [M+H]⁺.

### Example 257

### 2-(5-methoxy-1H-pyrazol-4-yl)-4-(2,8-diazaspiro[4.5]decan-8-yl)pyrido[3,4-d]pyrimidine (Compound 257) formate

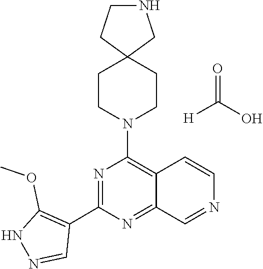

Following the procedure described in Example 241, Steps 1-3 and making non-critical variations as required to replace the aldehyde with 3-methoxy-1H-pyrazole-4-carbaldehyde was obtained the titled compound as pale yellow solid. ¹H NMR (400 MHz, DMSO-d₆) 12.32 (s, 1H), 9.02 (s, 1H), 8.41 (d, J=5.6 Hz, 1H), 8.20 (s, 1H), 7.73 (d, J=5.3 Hz, 1H), 3.89 (s, 3H), 3.88-3.71 (m, 4H), 3.20 (br s, 2H), 2.89 (t, J=7.1 Hz, 1H), 2.70 (br s, 1H), 1.74-1.58 (m, 6H). LCMS (ESI) m/z: 366.3, [M+H]⁺.

### Example 258

### N,N-Dimethyl-2-(pyridin-4-yl)-4-(2,8-diazaspiro[4.5]decan-8-yl)pyrido[3,4-d]pyrimidin-5-amine (Compound 258)

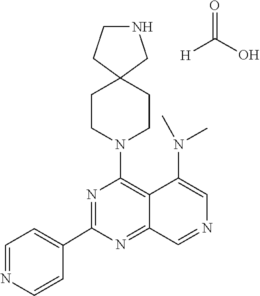

Following the procedure described in Example 233, Steps 3-5 and making non-critical variations as required to replace the methoxide with N,N-dimethylamine was obtained the titled compound as pale yellow solid. ¹H NMR (400 MHz, DMSO-d₆) δ 8.71 (d, J=5.0 Hz, 2H), 8.60 (s, 1H), 8.36 (br s, 1H), 8.28 (d, J=5.0 Hz, 2H), 8.12 (s, 1H), 3.82-3.32 (m, 4H), 3.24 (t, J=7.1 Hz, 2H), 3.04 (s, 2H), 2.86 (s, 6H), 1.92-1.85 (m, 2H), 1.84-1.29 (m, 4H). UPLCMS (ESI) m/z: 390.81 [M+H]⁺.

### Example 259

### 4-(2-methyl-2,8-diazaspiro[4.5]decan-8-yl)-2-(pyridin-4-yl)pyrido[3,4-d]pyrimidin-5-ol (Compound 259) triformate

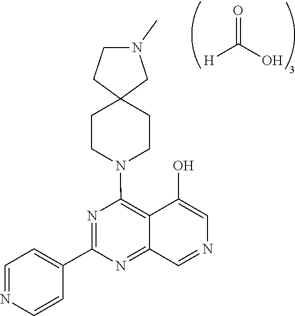

Following the procedure described in Example 252 and making non-critical variations as required to replace the substrate with 2-(Pyridin-4-yl)-4-(2,8-diazaspiro[4.5]decan-8-yl)pyrido[3,4-d]pyrimidin-5-ol was obtained the titled compound as pale yellow solid. ¹H NMR (400 MHz, DMSO-d₆) δ 8.69 (dd, J=4.5, 1.5 Hz, 2H), 8.40 (s, 3H), 8.24 (dd, J=4.5, 1.5 Hz, 2H), 8.16 (s, 1H), 7.90 (s, 1H), 3.76-3.70 (m, 6H), 2.34 (s, 2H), 2.20 (s, 3H), 1.71-1.56 (m, 6H). LCMS (ESI) m/z: 377.3 [M+H]⁺.

### Example 260

### 5-(Oxetan-3-yloxy)-2-(pyridin-4-yl)-4-(2,8-diazaspiro[4.5]decan-8-yl)pyrido[3,4-d]pyrimidine (Compound 260)

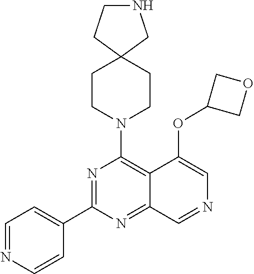 [SMILES: c1cc(-c2nc(N3CCC4(CCNC4)CC3)c3c(OC4COC4)cncc3n2)ccn1]

### Step 1: tert-Butyl 8-(5-fluoro-2-(pyridin-4-yl)pyrido[3,4-d]pyrimidin-4-yl)-2,8-diazaspiro[4.5]decane-2-carboxylate

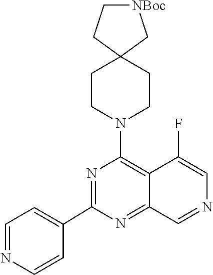 [SMILES: CC(C)(C)OC(=O)N1CCC2(CCN(c3nc(-c4ccncc4)nc4cncc(F)c34)CC2)C1]

A solution of 5-fluoro-2-(pyridin-4-yl)pyrido[3,4-d]pyrimidin-4-ol (300 mg, 0.1.24 mmol), DIPEA (0.65 mL, 3.71 mmol), 2,4,6-triisopropylbenzenesulfonyl chloride (580 mg, 1.86 mmol), and 4-dimethylaminopyridine (30.6 mg, 0.25 mmol) in DMA (6.2 mL) was stirred at room temperature for 30 minutes. To the mixture was added tert-Butyl 2,8-diazaspiro[4.5]decane-2-carboxylate (376 mg, 1.49 mmol), and the reaction was allowed to stir at room temperature for 24 hour. After monitoring the reaction via LCMS, the reaction had gone to completion at this time. The mixture was transferred to a separatory funnel, and diluted with DCM (50 mL) and water (50 mL). The layers were separated, and the aqueous layer was extracted with further DCM (3×30 mL). The combined organic extracts dried over Na₂SO₄, filtered, and concentrated in vacuo. The crude residue was purified by silica gel chromatography (solvent gradient: 0-10% MeOH in DCM) to give the title compound as white solid (530 mg, 92% yield). ¹H NMR (400 MHz, MeOD) δ 9.09 (s 1H), 8.71 (dd, J=4.4, 1.7 Hz, 2H), 8.50-8.43 (m, 3H), 4.00-3.75 (m, 4H), 3.51-3.42 (m, 2H), 3.35 (s, 2H), 1.92 (t, J=7.2 Hz, 2H), 1.85-1.75 (m, 4H), 1.47 (s, 9H). LCMS (ESI) m/z: 465.1 [M+H]⁺.

### Step 2: tert-Butyl 8-(5-(oxetan-3-yloxy)-2-(pyridin-4-yl)pyrido[3,4-d]pyrimidin-4-yl)-2,8-diazaspiro[4.5]decane-2-carboxylate

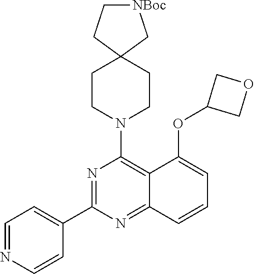 [SMILES: CC(C)(C)OC(=O)N1CCC2(CCN(c3nc(-c4ccncc4)nc4cccc(OC5COC5)c34)CC2)C1]

To a suspension of sodium hydride (60% dispersion in mineral oil, 19.4 mg, 0.484 mmol) in THF (0.16 mL) was added oxetan-3-ol (40.8 μL, 0.484 mmol) under nitrogen atmosphere at 0° C., and the mixture was stirred at 0° C. for 30 min. A solution of tert-butyl 8-(5-fluoro-2-(pyridin-4-yl)pyrido[3,4-d]pyrimidin-4-yl)-2,8-diazaspiro[4.5]decane-2-carboxylate (75.0 mg, 0.161 mmol) in NMP (0.8 mL) was added, and the reaction was further stirred at room temperature for 2 hours. The mixture was transferred to a separatory funnel, and diluted with DCM (5 mL) and water (5 mL). The layers were separated, and the aqueous layer was extracted further with DCM (3×3 mL). The combined organic extracts dried over Na₂SO₄, filtered, and concentrated in vacuo to give the title compound (80 mg, crude) as a brown solid that required no further purification. LCMS (ESI) m/z: 519.1 [M+H]⁺.

### Step 3: 5-(Oxetan-3-yloxy)-2-(pyridin-4-yl)-4-(2,8-diazaspiro[4.5]decan-8-yl)pyrido[3,4-d]pyrimidine

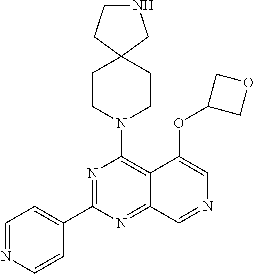 [SMILES: c1cc(-c2nc(N3CCC4(CCNC4)CC3)c3c(OC4COC4)cncc3n2)ccn1]

tert-Butyl 8-(5-(oxetan-3-yloxy)-2-(pyridin-4-yl)pyrido[3,4-d]pyrimidin-4-yl)-2,8-diazaspiro[4.5]decane-2-carboxylate (80 mg, crurde) was dissolved in 2 mL DCM and 0.3 mL TFA. The mixture was stirred at room temperature for 3 hours. The reaction mixture was then concentrated in vacuo, and then concentrated 2× further from DCM (5 mL) to remove residual TFA. The crude residue was then purified by HPLC to furnish the titled compound (17.3 mg, 26% yield) as white solid. ¹H NMR (400 MHz, DMSO-d₆) δ 8.85 (s, 1H), 8.80-8.74 (m, 2H), 8.35-8.27 (m, 2H), 7.89 (s, 1H), 5.57 (p, J=6.0 Hz, 1H), 5.04 (dd, J=6.8, 6.0 Hz, 2H), 4.78-4.70 (m, 2H), 3.90-3.70 (m, 4H), 2.84 (t, J=7.1 Hz, 2H), 2.65 (s, 2H), 1.79 (t, J=7.1 Hz, 1H), 1.69-1.55 (m, 5H). LCMS (ESI) m/z: 419.1 [M+H].

### Example 261

### 4-(2-(prop-2-yn-1-yl)-2,8-diazaspiro[4.5]decan-8-yl)-2-(pyridin-4-yl)pyrido[3,4-d]pyrimidine (Compound 261)

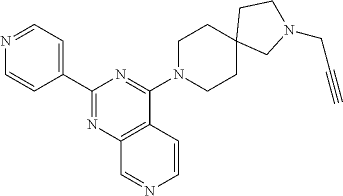 [SMILES: C#CCN1CCC2(CCN(c3nc(-c4ccncc4)nc4cnccc34)CC2)C1]

Following the procedure described in Example 256 and making non-critical variations as required to replace 4-bromobut-1-yne with 3-bromoprop-1-yne, the title compound was obtained as a white solid. ¹H NMR (400 MHz, DMSO-d₆) δ 9.24 (s, 1H), 8.79-8.73 (m, 2H), 8.57 (d, J=5.6 Hz, 1H), 8.35-8.28 (m, 2H), 7.87 (d, J=5.6 Hz, 1H), 4.03-3.82 (m, 4H), 3.37-3.36 (m, 2H), 3.14 (t, J=2.4 Hz, 1H), 2.64 (t, J=6.8 Hz, 2H), 2.52 (s, 2H), 1.83-1.66 (m, 6H). LCMS (ESI) m/z: 385.2 [M+H].

### Example 262

### 8-chloro-2-(5-methyl-1H-pyrazol-4-yl)-4-(2-methyl-2,8-diazaspiro[4.5]decan-8-yl)pyrido[3,4-d]pyrimidine (Compound 262)

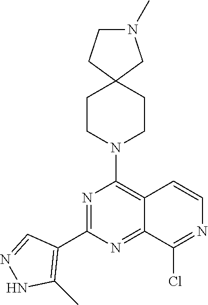 [SMILES: Cc1[nH]ncc1-c1nc(N2CCC3(CCN(C)C3)CC2)c2ccnc(Cl)c2n1]

Following the procedure described in Example 102 and making non-critical variations as required to replace 4-(2,8-diazaspiro[4.5]decan-8-yl)-2-(4-pyridyl)pyrido[3,4-d]pyrimidine hydrochloride with 8-chloro-2-(5-methyl-1H-pyrazol-4-yl)-4-(2,8-diazaspiro[4.5]decan-8-yl)pyrido[3,4-d]pyrimidine, the title compound was obtained as a white solid. ¹H NMR (400 MHz, DMSO) δ 12.91 (s, 1H), 8.20 (d, J=5.6 Hz, 1H), 8.12-8.03 (m, 1H), 7.79-7.72 (m, 2H), 3.90-3.81 (m, 2H), 3.81-3.72 (m, 2H), 2.76-2.61 (m, 3H), 2.49-2.47 (m, 1H), 2.38 (s, 2H), 2.23 (s, 3H), 1.79-1.64 (m, 6H). LCMS (ESI) m/z: 398.2 [M+H].

### Example 263

### 5-(2-methoxyethoxy)-2-(pyridin-4-yl)-4-(2,8-diazaspiro[4.5]decan-8-yl)pyrido[3,4-d]pyrimidine (Compound 263)

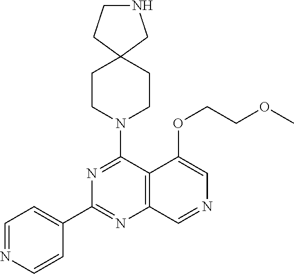 [SMILES: COCCOc1cncc2nc(-c3ccncc3)nc(N3CCC4(CCNC4)CC3)c12]

### Step 1: 2-(pyridin-4-yl)pyrido[3,4-d]pyrimidine-4,5-diol

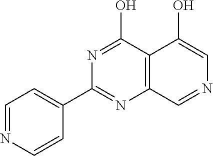 [SMILES: Oc1cncc2nc(-c3ccncc3)nc(O)c12]

A mixture of 5-methoxy-2-(4-pyridyl)pyrido[3,4-d]pyrimidin-4-ol (2 g, 7.87 mmol) and pyridine hydrochloride (7 g, 60.57 mmol) in a sealed tube was heated to 170° C. for 1 h under microwave. After cooling to room temperature, the mixture was dissolved in water (20 mL) and basified with 2M NaOH to pH 7 at room temperature, then acidified with AcOH to pH 4. The resulting brown precipitate was filtered and washed with water (10 mL×2) to give the title compound (1.2 g, 64%) as a brown solid. LCMS (ESI) m/z: 241.2 [M+H]⁺.

### Step 2: tert-butyl 8-(2-(pyridin-4-yl)-5-(((2,4,6-triisopropylphenyl)sulfonyl)oxy)pyrido[3,4-d]pyrimidin-4-yl)-2,8-diazaspiro[4.5]decane-2-carboxylate

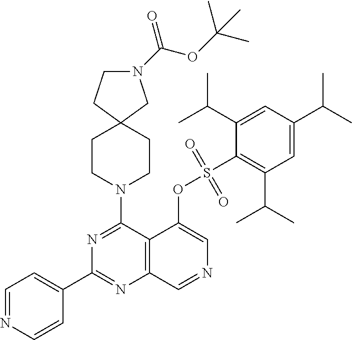 [SMILES: CC(C)c1cc(C(C)C)c(S(=O)(=O)Oc2cncc3nc(-c4ccncc4)nc(N4CCC5(CCN(C(=O)OC(C)(C)C)C5)CC4)c23)c(C(C)C)c1]

To a solution of 2-(4-pyridyl)pyrido[3,4-d]pyrimidine-4,5-diol (1.2 g, 5 mmol) in DMAc (25 mL) was added N,N-diisopropylethylamine (3.5 mL, 20 mmol) and 2,4,6-triisopropylbenzenesulfonyl chloride (4.5 g, 15 mmol). The reaction mixture was stirred at room temperature for 3 h and then tert-butyl 2,8-diazaspiro[4.5]decane-2-carboxylate (1.2 g, 5 mmol) was added to this reaction mixture. The reaction mixture was stirred at room temperature for 16 h. The mixture was diluted with EtOAc (250 mL), washed with water (150 mL×3) and brine (150 mL). The organic layer was dried over anhydrous Na₂SO₄, filtered and concentrated in vacuo. The crude residue was purified by silica gel chromatography (solvent gradient: 0-5% MeOH in DCM) to give the title compound (270 mg, 10%) as a yellow solid. LCMS (ESI) m/z: 729.1 [M+H]⁺.

### Step 3: tert-butyl 8-(5-hydroxy-2-(pyridin-4-yl)pyrido[3,4-d]pyrimidin-4-yl)-2,8-diazaspiro[4.5]decane-2-carboxylate

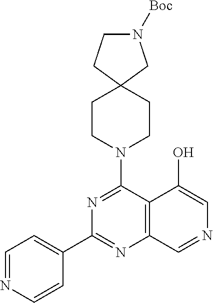 [SMILES: CC(C)(C)OC(=O)N1CCC2(CCN(c3nc(-c4ccncc4)nc4cncc(O)c34)CC2)C1]

To a solution of tert-butyl 8-(2-(pyridin-4-yl)-5-(((2,4,6-triisopropylphenyl)sulfonyl)oxy)pyrido[3,4-d]pyrimidin-4-yl)-2,8-diazaspiro[4.5]decane-2-carboxylate (270 mg, 0.37 mmol) in dioxane (2 mL) was added NaOH as a 2M solution in H₂O (0.74 mL, 1.48 mmol). The reaction mixture was stirred at 60° C. for 2 h. After cooling to room temperature, the mixture was diluted with water (5 mL) and then neutralized to pH=6 by HCl (0.1 M), extracted with EtOAc (10 mL×2). The combined organic layers were washed with brine (10 mL), dried over anhydrous Na₂SO₄, filtered and concentrated in vacuo. The residue was purified by prep-TLC (EtOAc) to give the title compound (150 mg, 66%) as yellow oil. LCMS (ESI) m/z: 463.3 [M+H]⁺.

### Step 4: tert-butyl 8-(5-(2-methoxyethoxy)-2-(pyridin-4-yl)pyrido[3,4-d]pyrimidin-4-yl)-2,8-diazaspiro[4.5]decane-2-carboxylate

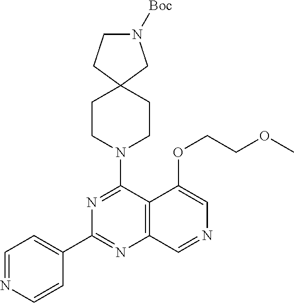 [SMILES: COCCOc1cncc2nc(-c3ccncc3)nc(N3CCC4(CCN(C(=O)OC(C)(C)C)C4)CC3)c12]

To a solution of tert-butyl 8-(5-hydroxy-2-(pyridin-4-yl)pyrido[3,4-d]pyrimidin-4-yl)-2,8-diazaspiro[4.5]decane-2-carboxylate (85 mg, 0.14 mmol) in DMF (1 mL) was added potassium carbonate (40 mg, 0.29 mmol) and 2-bromoethyl methyl ether (0.03 mL, 0.29 mmol). The mixture was stirred at room temperature for 16 h. The mixture was diluted with water (3 mL) and extracted with EtOAc (5 mL×2). The combined organic layers were washed with brine (10 mL), dried over anhydrous Na₂SO₄, filtered and concentrated in vacuo. The residue was purified by prep-TLC (DCM/MeOH=10:1) to give the title compound (28 mg, 39%) as yellow oil. LCMS (ESI) m/z: 521.3 [M+H]⁺.

### Step 5: 5-(2-methoxyethoxy)-2-(pyridin-4-yl)-4-(2,8-diazaspiro[4.5]decan-8-yl)pyrido[3,4-d]pyrimidine

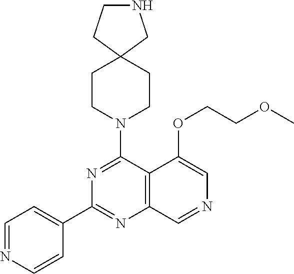 [SMILES: COCCOc1cncc2nc(-c3ccncc3)nc(N3CCC4(CCNC4)CC3)c12]

Following the procedure described in Example 157, step 7 and making non-critical variations as required to replace tert-butyl 8-(6-benzyl-2-(pyridin-4-yl)pyrido[3,4-d]pyrimidin-4-yl)-2,8-diazaspiro[4.5]decane-2-carboxylate with tert-butyl 8-(5-(2-methoxyethoxy)-2-(pyridin-4-yl)pyrido[3,4-d]pyrimidin-4-yl)-2,8-diazaspiro[4.5]decane-2-carboxylate, the title compound was obtained (12 mg, 42%) as a yellow solid. ¹H NMR (400 MHz, DMSO-d₆) δ 8.84-8.81 (m, 1H), 8.79-8.74 (m, 2H), 8.35-8.33 (m, 1H), 8.31-8.29 (m, 2H), 4.45-4.39 (m, 2H), 3.80-3.78 (m, 2H), 3.77-3.67 (m, 4H), 3.35 (s, 3H), 3.08 (t, J=7.2 Hz, 2H), 2.87 (s, 2H), 1.75 (t, J=7.6 Hz, 2H), 1.70-1.63 (m, 4H). LCMS (ESI) m/z: 421.0 [M+H]⁺.

### Example 264

### 5-methoxy-2-(3-methyl-1H-pyrazol-4-yl)-4-(2-methyl-2,8-diazaspiro[4.5]decan-8-yl)pyrido[3,4-d]pyrimidine (Compound 264)

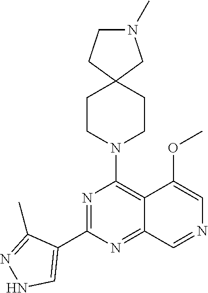 [SMILES: COc1cncc2nc(-c3c[nH]nc3C)nc(N3CCC4(CCN(C)C4)CC3)c12]

### Step 1: 3-amino-5-methoxyisonicotinamide

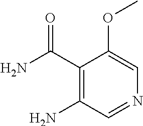

A solution of 3-amino-5-methoxy-pyridine-4-carboxylic acid (2 g, 11.9 mmol) (prepared according to the procedure in Chem. Pharm. Bull., 1982, 30, 1257), di-tert-butyl dicarbonate (3.2 mL, 13.9 mmol), pyridine (1.9 mL, 23.8 mmol) and NH₄CO₃ (1.2 g, 12.5 mmol) in dioxane (100 mL) was stirred at room temperature for 16 h. The reaction mixture was concentrated in vacuo. The crude residue was purified by silica gel chromatography (solvent gradient: 0-100% EtOAc in petroleum ether) to give the title compound (630 mg, 32%) as a yellow solid. LCMS (ESI) m/z: 168.1 [M+H]⁺.

### Step 2: 5-methoxy-2-(3-methyl-1-((2-(trimethylsilyl)ethoxy)methyl)-1H-pyrazol-4-yl)pyrido[3,4-d]pyrimidin-4-ol

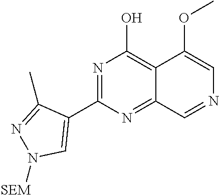 [SMILES: COc1cncc2nc(-c3cn(COCC[Si](C)(C)C)nc3C)nc(O)c12]

Following the procedure described in Example 142, step 2 and making non-critical variations as required to replace 3-aminoisonicotinamide with 3-amino-5-methoxyisonicotinamide, the title compound was obtained (2.1 g, 45%) as a yellow solid. LCMS (ESI) m/z: 388.2 [M+H]⁺.

### Step 3: tert-butyl 2-methyl-2,8-diazaspiro[4.5]decane-8-carboxylate

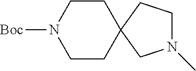 [SMILES: CN1CCC2(CCN(C(=O)OC(C)(C)C)CC2)C1]

To a solution of tert-butyl 2,8-diazaspiro[4.5]decane-8-carboxylate (1 g, 4.16 mmol) in MeOH (20 mL) was added formaldehyde (0.37 mL, 4.99 mmol, 37% in water) and acetic acid (24 uL, 0.42 mmol). The mixture was stirred at room temperature for 10 min before the addition of sodium triacetoxyborohydride (2.65 g, 12.48 mmol). The mixture was stirred at room temperature for 16 h. The reaction mixture was concentrated in vacuo. The crude residue was dissolved in EtOAc (120 mL), washed with sat. aq. NaHCO₃ (50 mL) and brine (50 mL). The organic layer was dried over anhydrous Na₂SO₄, filtered and concentrated in vacuo to give the title compound (1 g, crude) as yellow oil that required no further purification. LCMS (ESI) m/z: 255.1 [M+H]⁺.

### Step 4: 2-methyl-2,8-diazaspiro[4.5]decane hydrochloride

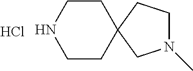

To a solution of tert-butyl 2-methyl-2,8-diazaspiro[4.5]decane-8-carboxylate (1 g, 3.93 mmol) in EtOAc (6 mL) was added 4M HCl in EtOAc (6 mL, 22 mmol). The mixture was stirred at room temperature for 2 h. The mixture was concentrated in vacuo to give the title compound (750 mg, crude) as a yellow solid that required no further purification. LCMS (ESI) m/z: 155.2 [M+H]⁺.

### Step 5: 5-methoxy-2-(3-methyl-1-((2-(trimethylsilyl)ethoxy)methyl)-1H-pyrazol-4-yl)-4-(2-methyl-2,8-diazaspiro[4.5]decan-8-yl)pyrido[3,4-d]pyrimidine

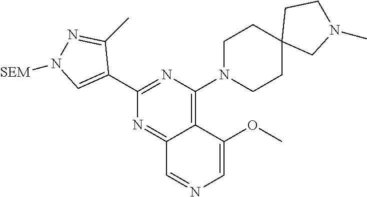 [SMILES: COc1cncc2nc(-c3cn(COCC[Si](C)(C)C)nc3C)nc(N3CCC4(CCN(C)C4)CC3)c12]

Following the procedure described in Example 209, step 3 and making non-critical variations as required to replace 2-(pyridin-4-yl)-6-(trifluoromethyl)pyrido[3,4-d]pyrimidin-4-ol and tert-butyl 2,8-diazaspiro[4.5]decane-2-carboxylate with 5-methoxy-2-(3-methyl-1-((2-(trimethylsilyl)ethoxy)methyl)-1H-pyrazol-4-yl)pyrido[3,4-d]pyrimidin-4-ol and 2-methyl-2,8-diazaspiro[4.5]decane, the title compound was obtained (56 mg, 40%) as a yellow solid. LCMS (ESI) m/z: 524.3 [M+H]⁺.

### Step 6: 5-methoxy-2-(3-methyl-1H-pyrazol-4-yl)-4-(2-methyl-2,8-diazaspiro[4.5]decan-8-yl)pyrido[3,4-d]pyrimidine

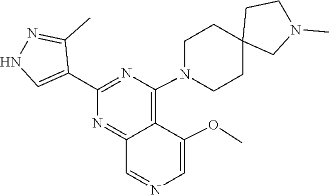 [SMILES: COc1cncc2nc(-c3c[nH]nc3C)nc(N3CCC4(CCN(C)C4)CC3)c12]

To a solution of 5-methoxy-2-(3-methyl-1-((2-(trimethylsilyl)ethoxy)methyl)-1H-pyrazol-4-yl)-4-(2-methyl-2,8-diazaspiro[4.5]decan-8-yl)pyrido[3,4-d]pyrimidine (56 mg, 0.11 mmol) in DCM (1 mL) was added trifluoroacetic acid (0.5 mL, 6.5 mmol). The mixture was stirred at room temperature for 2 h. The mixture was concentrated in vacuo. The crude residue was purified by reverse phase chromatography (acetonitrile 40-70%/0.05% NH₃·H₂O+10 mM NH₄HCO₃ in water) to give the title compound (5 mg, 13%) as a white solid. ¹H NMR (400 MHz, DMSO-d₆) δ 12.86 (s, 1H), 8.65 (s, 1H), 8.17 (s, 1H), 8.04 (s, 1H) 4.03 (s, 3H), 3.64-3.49 (m, 4H), 2.67 (s, 3H), 2.58-2.51 (m, 2H), 2.36 (s, 2H), 2.22 (s, 3H), 1.72-1.59 (m, 6H). LCMS (ESI) m/z: 394.3 [M+H]⁺.

### Example 265

### 1-(8-(5-methoxy-2-(3-methyl-1H-pyrazol-4-yl)pyrido[3,4-d]pyrimidin-4-yl)-2,8-diazaspiro[4.5]decan-2-yl)-2-methylpropan-2-ol Compound 265

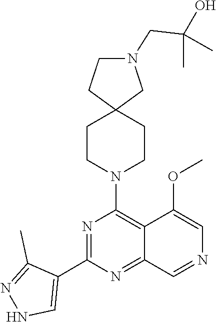 [SMILES: COc1cncc2nc(-c3c[nH]nc3C)nc(N3CCC4(CCN(CC(C)(C)O)C4)CC3)c12]

### 1-(8-(5-methoxy-2-(3-methyl-1H-pyrazol-4-yl)pyrido[3,4-d]pyrimidin-4-yl)-2,8-diazaspiro[4.5]decan-2-yl)-2-methylpropan-2-ol

### Step 1: tert-butyl 2-(2-hydroxy-2-methylpropyl)-2,8-diazaspiro[4.5]decane-8-carboxylate

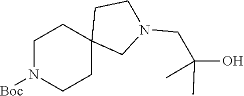 [SMILES: CC(C)(O)CN1CCC2(CCN(C(=O)OC(C)(C)C)CC2)C1]

Following the procedure described in Example 250, step 6 and making non-critical variations as required to replace 5-methoxy-2-(pyridin-4-yl)-4-(2,8-diazaspiro[4.5]decan-8-yl)pyrido[3,4-d]pyrimidine hydrochloride with tert-butyl 2,8-diazaspiro[4.5]decane-8-carboxylate, the title compound was obtained (350 mg, crude) as yellow oil. LCMS (ESI) m/z: 313.3 [M+H]⁺.

### Step 2: 1-(8-(5-methoxy-2-(3-methyl-1H-pyrazol-4-yl)pyrido[3,4-d]pyrimidin-4-yl)-2,8-diazaspiro[4.5]decan-2-yl)-2-methylpropan-2-ol

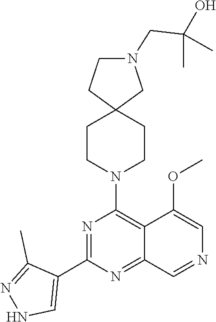 [SMILES: COc1cncc2nc(-c3c[nH]nc3C)nc(N3CCC4(CCN(CC(C)(C)O)C4)CC3)c12]

Following the procedure described in Example 264, step 4-6 and making non-critical variations as required to replace tert-butyl 2-methyl-2,8-diazaspiro[4.5]decane-8-carboxylate with tert-butyl 2-(2-hydroxy-2-methylpropyl)-2,8-diazaspiro[4.5]decane-8-carboxylate, the title compound was obtained (21 mg, 9%) as a yellow solid. ¹H NMR (400 MHz, DMSO-d₆) δ 12.82 (s, 1H), 8.64 (s, 1H), 8.17 (s, 1H), 8.03 (s, 1H), 4.03 (s, 3H), 3.50-3.62 (m, 4H), 2.66 (s, 3H), 2.56-2.51 (m, 4H), 2.34 (s, 2H), 1.60-1.69 (m, 6H), 1.09 (s, 6H). LCMS (ESI) m/z: 452.3 [M+H]⁺.

### Example 266

### 8-chloro-4-(2-methyl-2,8-diazaspiro[4.5]decan-8-yl)-2-(pyridin-4-yl)pyrido[3,4-d]pyrimidine (Compound 266)

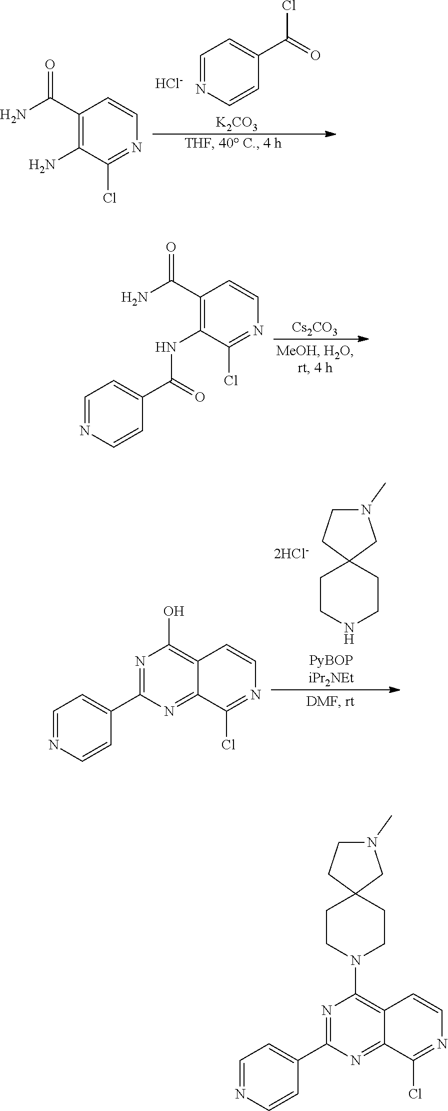

### Step 1: 2-chloro-3-(isonicotinamido)isonicotinamide

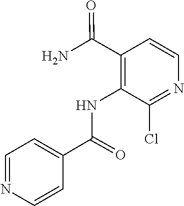 [SMILES: NC(=O)c1ccnc(Cl)c1NC(=O)c1ccncc1]

To a solution of 3-amino-2-chloro-pyridine-4-carboxamide (7.0 g, 40.8 mmol) and isonicotinoyl chloride hydrochloride (8.7 g, 49.0 mmol) in THF (100 mL) was added K₂CO₃ (14.5 g, 81.6 mmol). The reaction mixture was heated to 40° C. for 4 h under nitrogen atmosphere. After cooling to room temperature, the solvent was removed under reduced pressure and water (50 mL) was added. The resulting solid was filtered, washed with water (30 mL×2) and the collected, dried with an azeotrope with toluene to afford the title compound (9 g, 80%) as a white solid which was used directly without further purification.

### Step 2: 8-chloro-2-(pyridin-4-yl)pyrido[3,4-d]pyrimidin-4-ol

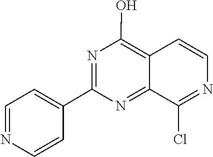 [SMILES: Oc1nc(-c2ccncc2)nc2c(Cl)nccc12]

To a solution of 2-chloro-3-(isonicotinamido)isonicotinamide (9 g, 32.5 mmol) in MeOH (300 mL) was added a solution of Cs₂CO₃ (32 g, 98.0 mmol) in water (50 mL). The reaction mixture was stirred at room temperature for 16 h. MeOH was removed in vacuo and the residue was diluted with water (100 mL). Acetic acid (20 mL) was added and the mixture was stirred at room temperature for 20 min, the resulting white precipitate was filtered and washed with water (30 mL×2). The solid was dried in vacuo to give the title compound (6 g, 71%) as a white solid. ¹H NMR (400 MHz, DMSO-d₆) δ 13.30 (s, 1H), 8.82 (d, J=6.0 Hz, 2H), 8.44 (d, J=5.2 Hz, 1H), 8.11 (d, J=6.0 Hz, 2H), 7.98 (d, J=5.6 Hz, 1H). LCMS (ESI) m/z: 259.2 [M+H]⁺.

### Step 3: 8-chloro-4-(2-methyl-2,8-diazaspiro[4.5]decan-8-yl)-2-(pyridin-4-yl)pyrido[3,4-d]pyrimidine

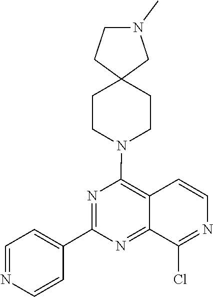 [SMILES: CN1CCC2(CCN(c3nc(-c4ccncc4)nc4c(Cl)nccc34)CC2)C1]

To a 20-mL vial was added 8-chloro-2-(4-pyridyl)pyrido[3,4-d]pyrimidin-4-ol (200 mg, 0.773 mmol, 200 mg, 1 equiv.) and PyBOP (622.23 mg, 1.1598 mmol, 1.5 equiv.), was fitted with a septum cap and purged with N₂. DMF (3.9 mL, 0.2 M) added followed by DIPEA (0.34 mL, 1.93 mmol, 2.5 equiv.). Let stir at room temperature for 10 min. 2-methyl-2,8-diazaspiro[4.5]decane dihydrochloride (222 mg, 0.928 mmol, 1.2 equiv.) added; reaction stirred at room temperature for 16 hours. The reaction was monitored by LCMS and had not gone to conversion, so additional DIPEA (0.34 mL, 1.93 mmol, 2.5 equiv.) added, and the reaction was allowed to stir for an additional 5 h at room temperature. The reaction was then quenched with water, and extracted with EtOAc (4×). The combined organic extracts dried over Na₂SO₄, filtered through a silica gel plug and flushed with EtOAc, and concentrated in vacuo. The crude residue was dissolved in MeOH; passed through a 10 g SCX-2 cartridge; the initial MeOH fraction was discarded. Then, switched to a new flask and flushed with 5%₀NH₃/MeOH solution and concentrated in vacuo to give the crude residue. The crude residue was purified by reverse phase chromatography (acetonitrile 5-50%/0.1% formic acid in water) to give 8-chloro-4-(2-methyl-2,8-diazaspiro[4.5]decan-8-yl)-2-(4-pyridyl)pyrido[3,4-d]pyrimidine (24.03 mg, 0.06085 mmol, 24.03 mg, 7.870% Yield). ¹H NMR (400 MHz, DMSO) δ 8.82-8.76 (m, 2H), 8.39-8.32 (m, 3H), 7.89 (d, J=5.6 Hz, 1H), 4.05-3.96 (m, 2H), 3.94-3.84 (m, 2H), 2.69-2.58 (m, 4H), 2.32 (s, 3H), 1.82-1.67 (m, 6H). LCMS (ESI) m/z: 395.1 [M+H]⁺.

### Example 267

### 5-(fluoromethoxy)-2-(pyridin-4-yl)-4-(2,8-diazaspiro[4.5]decan-8-yl)pyrido[3,4-d]pyrimidine (Compound 267)

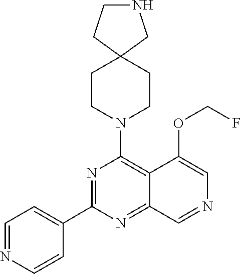 [SMILES: FCOc1cncc2nc(-c3ccncc3)nc(N3CCC4(CCNC4)CC3)c12]

### Step 1: tert-butyl 8-(5-(fluoromethoxy)-2-(pyridin-4-yl)pyrido[3,4-d]pyrimidin-4-yl)-2,8-diazaspiro[4.5]decane-2-carboxylate

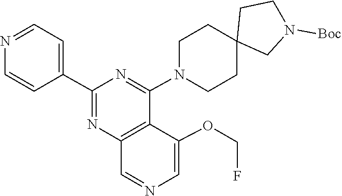 [SMILES: CC(C)(C)OC(=O)N1CCC2(CCN(c3nc(-c4ccncc4)nc4cncc(OCF)c34)CC2)C1]

To a solution of tert-butyl 8-(5-hydroxy-2-(pyridin-4-yl)pyrido[3,4-d]pyrimidin-4-yl)-2,8-diazaspiro[4.5]decane-2-carboxylate (65 mg, 0.14 mmol) in DMF (1 mL) was added bromo(fluoro)methane (0.04 mL, 0.58 mmol) and potassium carbonate (40 mg, 0.29 mmol). The mixture was stirred at room temperature for 16 h. The mixture was diluted with water (3 mL) and extracted with EtOAc (5 mL×2). The combined organic layers were washed with brine (10 mL), dried over anhydrous Na₂SO₄, filtered and concentrated in vacuo. The crude residue was purified by prep-TLC (DCM/MeOH=10:1) to give the title compound (10 mg, 14%) as yellow oil. LCMS (ESI) m/z: 517.3 [M+Na]⁺.

### Step 2: 5-(fluoromethoxy)-2-(pyridin-4-yl)-4-(2,8-diazaspiro[4.5]decan-8-yl)pyrido[3,4-d]pyrimidine

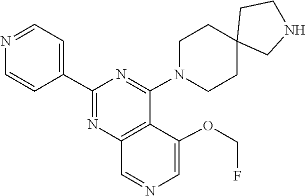 [SMILES: FCOc1cncc2nc(-c3ccncc3)nc(N3CCC4(CCNC4)CC3)c12]

Following the procedure described in Example 263, step 4-5 and making non-critical variations as required to replace 1-bromo-2-methoxyethane with bromofluoromethane, the title compound was obtained (3 mg, 37%) as a yellow solid. ¹H NMR (400 MHz, DMSO-d₆) δ 8.99 (s, 1H), 8.82-8.73 (m, 2H), 8.45 (s, 1H), 8.34-8.28 (m, 2H), 6.10 (d, J=52.8 Hz, 2H), 3.83-3.63 (m, 4H), 2.88-2.82 (m, 1H), 2.68-2.65 (m, 1H), 2.58-2.51 (m, 2H), 1.84-1.79 (m, 1H), 1.72-1.63 (m, 4H), 1.62-1.55 (m, 1H). LCMS (ESI) m/z: 395.2 [M+H].

### Example 268

### 2-Methyl-1-(8-(5-(oxetan-3-yloxy)-2-(pyridin-4-yl)pyrido[3,4-d]pyrimidin-4-yl)-2,8-diazaspiro[4.5]decan-2-yl)propan-2-o0 (Compound 268)

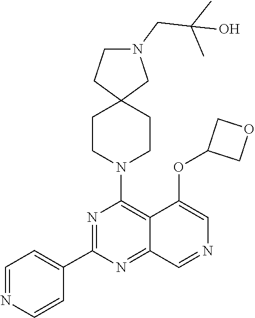 [SMILES: CC(C)(O)CN1CCC2(CCN(c3nc(-c4ccncc4)nc4cncc(OC5COC5)c34)CC2)C1]

To a solution of 5-(oxetan-3-yloxy)-2-(pyridin-4-yl)-4-(2,8-diazaspiro[4.5]decan-8-yl)pyrido[3,4-d]pyrimidine (43.0 mg, 0.103 mmol) in MeOH (0.70 mL) under nitrogen atmosphere was added isobutyleneoxide (27 μL, 0.308 mmol) and DIPEA (90 μL, 0.514 mmol). The mixture was stirred at 80° C. in a microwave apparatus for 2 h. After cooling to room temperature, the mixture was concentrated in vacuo. The crude residue was then purified by HPLC to fumish the titled compound (26.0 mg, 52% yield) as a white solid. ¹H NMR (400 MHz, DMSO-d₆) δ 8.85 (s, 1H), 8.79-8.74 (m, 2H), 8.33-8.27 (m, 2H), 7.88 (s, 1H), 5.57 (p, J=5.3 Hz, 1H), 5.09-5.00 (m, 2H), 4.73 (dd, J=7.6, 4.9 Hz, 2H), 4.01 (br s, 1H), 3.86-3.69 (m, 4H), 2.67 (t, J=7.0 Hz, 2H), 2.54 (s, 2H), 2.32 (s, 2H), 1.70-1.59 (m, 6H), 1.08 (s, 6H). LCMS (ESI) m/z: 491.2 [M+H]⁺.

### Example 269

### 4-(2-Methyl-2,8-diazaspiro[4.5]decan-8-yl)-5-(oxetan-3-yloxy)-2-(pyridin-4-yl)pyrido[3,4-d]pyrimidine (Compound 269)

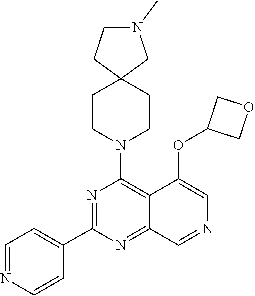 [SMILES: CN1CCC2(CCN(c3nc(-c4ccncc4)nc4cncc(OC5COC5)c34)CC2)C1]

### Step 1: 5-Fluoro-4-(2-methyl-2,8-diazaspiro[4.5]decan-8-yl)-2-(pyridin-4-yl)pyrido[3,4-d]pyrimidine

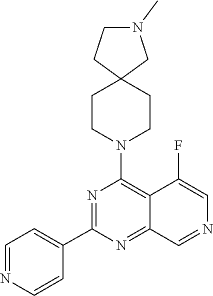 [SMILES: CN1CCC2(CCN(c3nc(-c4ccncc4)nc4cncc(F)c34)CC2)C1]

Following the procedure described in Example 260, Step 1 and making non-critical variations as required to replace the amine with 2-methyl-2,8-diazaspiro[4.5]decane dihydrochloride was obtained the titled compound as a white solid (500 mg, 64% yield). ¹H NMR (400 MHz, MeOD) δ 9.06 (s, 1H), 8.73-8.70 (m, 2H), 8.47-8.41 (m, 3H), 3.93-3.84 (m, 2H), 3.83-3.75 (m, 2H), 2.73 (t, J=6.9 Hz, 2H), 2.60 (s, 2H), 2.41 (s, 3H), 1.89-1.77 (m, 6H). LCMS (ESI) m/z: 379.0 [M+H]⁺.

### Step 2: 4-(2-Methyl-2,8-diazaspiro[4.5]decan-8-yl)-5-(oxetan-3-yloxy)-2-(pyridin-4-yl)pyrido[3,4-d]pyrimidine

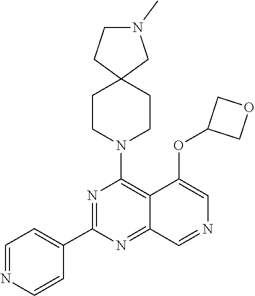 [SMILES: CN1CCC2(CCN(c3nc(-c4ccncc4)nc4cncc(OC5COC5)c34)CC2)C1]

Following the procedure described in Example 260, Step 2 and making non-critical variations as required to replace the aryl fluoride with 5-fluoro-4-(2-methyl-2,8-diazaspiro[4.5]decan-8-yl)-2-(pyridin-4-yl)pyrido[3,4-d]pyrimidine was obtained the titled compound as a white solid (18.0 mg, 63% yield). ¹H NMR (400 MHz, DMSO-d₆) δ 8.85 (s, 1H), 8.79-8.74 (m, 2H), 8.32-8.27 (m, 2H), 7.89 (s, 1H), 5.62-5.52 (m, 1H), 5.07-5.00 (m, 2H), 4.73 (dd, J=7.6, 4.9 Hz, 2H), 3.84-3.67 (m, 4H), 2.49-2.38 (m, 2H), 2.37 (s, 2H), 2.22 (s, 3H), 1.74-1.58 (m, 6H). LCMS (ESI) m/z: 433.1 [M+H]⁺.

### Example 270

### 4-(2-Methyl-2,8-diazaspiro[4.5]decan-8-yl)-5-(oxetan-3-ylmethoxy)-2-(pyridin-4-yl)pyrido[3,4-d]pyrimidine (Compound 270)

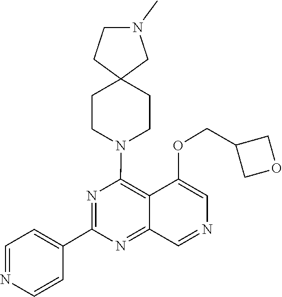 [SMILES: CN1CCC2(CCN(c3nc(-c4ccncc4)nc4cncc(OCC5COC5)c34)CC2)C1]

Following the procedure described in Example 260, Step 2 and making non-critical variations as required to replace the alcohol with oxetan-3-ylmethanol was obtained the titled compound (19.0 mg, 64% yield) as a white solid. ¹H NMR (400 MHz, DMSO-d₆) δ 8.83 (s, 1H), 8.79-8.73 (m, 2H), 8.37 (s, 1H), 8.32-8.26 (m, 2H), 4.77 (dd, J=7.9, 6.0 Hz, 2H), 4.53 (d, J=6.5 Hz, 2H), 4.47 (apparent t, J=6.1 Hz, 2H), 3.78-3.61 (m, 4H), 3.54 (ddt, J=7.9, 6.5, 6.1 Hz, 1H), 2.47 (t, J=6.9 Hz, 2H), 2.35 (s, 2H), 2.22 (s, 3H), 1.67-1.52 (m, 6H). LCMS (ESI) m/z: 447.2 [M+H]⁺.

### Example 271

### 2-methyl-1-(8-(2-(pyridin-4-yl)pyrido[3,4-d]pyrimidin-4-yl)-2,8-diazaspiro[4.5]decan-2-yl)but-3-yn-2-ol (Compound 271)

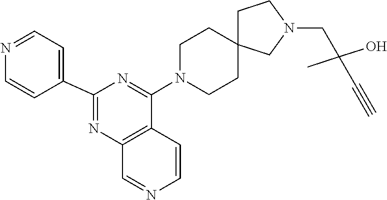 [SMILES: C#CC(C)(O)CN1CCC2(CCN(c3nc(-c4ccncc4)nc4cnccc34)CC2)C1]

### Step 1: 2-methyl-1-(8-(2-(pyridin-4-yl)pyrido[3,4-d]pyrimidin-4-yl)-2,8-diazaspiro[4.5]decan-2-yl)-4-(trimethylsilyl)but-3-yn-2-ol

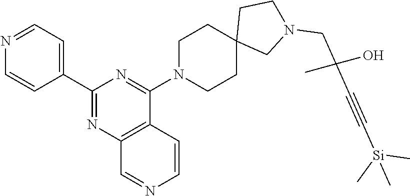 [SMILES: CC(O)(C#C[Si](C)(C)C)CN1CCC2(CCN(c3nc(-c4ccncc4)nc4cnccc34)CC2)C1]

To a solution of trimethyl((2-methyloxiran-2-yl)ethynyl)silane (134 mg, 0.87 mmol, prepared according to Angew. Chem. Int. Ed., 2013, 52, 13033), N,N-diisopropylethylamine (0.31 mL, 1.73 mmol) in EtOH (5 mL) was added 2-(pyridin-4-yl)-4-(2,8-diazaspiro[4.5]decan-8-yl)pyrido[3,4-d]pyrimidine hydrochloride (200 mg, 0.58 mmol). The mixture was heated to 80° C. for 3 h. The reaction mixture was quenched with sat. aq. NH₄Cl (20 mL), extracted with EtOAc (50 mL×2). The combined organic layers were dried over anhydrous Na₂SO₄, filtered and concentrated in vacuo. The residue was purified by silica gel chromatography (solvent gradient: 0-50% EtOAc in petroleum ether) to give the title compound (22 mg, 8%) as a yellow oil. LCMS (ESI) m/z: 501.2 [M+H]⁺.

### Step 2: 2-methyl-1-(8-(2-(pyridin-4-yl)pyrido[3,4-d]pyrimidin-4-yl)-2,8-diazaspiro[4.5]decan-2-yl)but-3-yn-2-ol

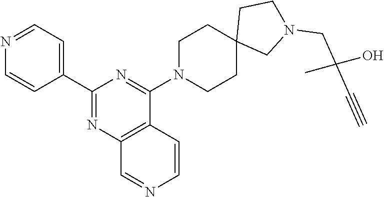 [SMILES: C#CC(C)(O)CN1CCC2(CCN(c3nc(-c4ccncc4)nc4cnccc34)CC2)C1]

To a solution of 2-methyl-1-(8-(2-(pyridin-4-yl)pyrido[3,4-d]pyrimidin-4-yl)-2,8-diazaspiro[4.5]decan-2-yl)-4-(trimethylsilyl)but-3-yn-2-ol (22 mg, 0.04 mmol) in THF (2 mL) was added TFA (0.18 mL, 0.18 mmol). The mixture was stirred at 0° C. for 1 h. The reaction mixture was quenched with water (50 mL), extracted with EtOAc (50 mL×2). The combined organic layers were dried over anhydrous Na₂SO₄, filtered and concentrated in vacuo. The crude residue was purified by prep-TLC (EtOAc/petroleum ether=1:1) to give the title compound (13 mg, 67%) as a white solid. ¹H NMR (400 MHz, DMSO-d₆) δ 9.25 (s, 1H), 8.80-8.74 (m, 2H), 8.58 (d, J=6.0 Hz, 1H), 8.35-8.30 (m, 2H), 7.89 (d, J=5.6 Hz, 1H), 5.18 (s, 1H), 4.00-3.86 (m, 4H), 3.21 (s, 1H), 2.82-2.72 (m, 2H), 2.66-2.61 (m, 2H), 2.60-2.53 (m, 2H), 1.83-1.72 (m, 4H), 1.70-1.63 (m, 2H), 1.36 (s, 3H). LCMS (ESI) m/z: 429.1 [M+H]⁺.

### Example 272

### 8-chloro-2-(pyridin-4-yl)-4-(2,8-diazaspiro[4.5]decan-8-yl)pyrido[3,4-d]pyrimidine trifluoro acetate (Compound 272)

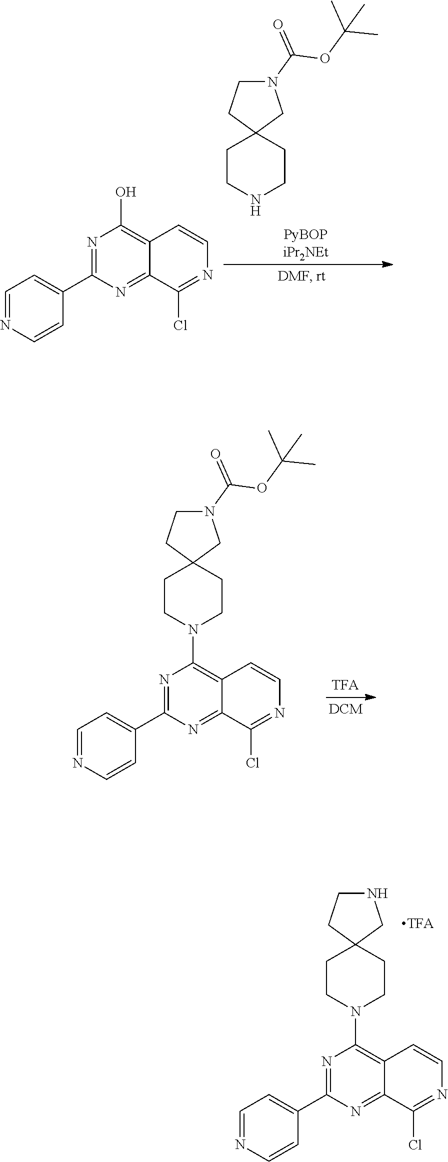 [SMILES: Oc1nc(-c2ccncc2)nc2c(Cl)nccc12 ; CC(C)(C)OC(=O)N1CCC2(CCNCC2)C1 ; CC(C)(C)OC(=O)N1CCC2(CCN(c3nc(-c4ccncc4)nc4c(Cl)nccc34)CC2)C1 ; Clc1nccc2c(N3CCC4(CCNC4)CC3)nc(-c3ccncc3)nc12]

### Step 1: tert-butyl 8-(8-chloro-2-(pyridin-4-yl)pyrido[3,4-d]pyrimidin-4-yl)-2,8-diazaspiro[4.5]decane-2-carboxylate

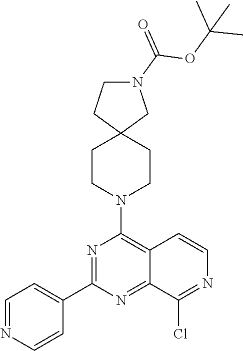 [SMILES: CC(C)(C)OC(=O)N1CCC2(CCN(c3nc(-c4ccncc4)nc4c(Cl)nccc34)CC2)C1]

To a 20-mL vial was added 8-chloro-2-(4-pyridyl)pyrido[3,4-d]pyrimidin-4-ol (200 mg, 0.773 mmol, 1 equiv.), PYBOP (622 mg, 1.16 mmol, 1.5 equiv.) fitted with a septum cap, and purged with N₂. DMF (3.87 mL, 0.2 M) added followed by DIPEA (0.34 mL, 1.93 mmol, 2.5 equiv.). Let stir at room temperature for 10 min. tert-butyl 2,8-diazaspiro[4.5]decane-2-carboxylate (293 mg, 1.16 mmol, 1.5 equiv.) added; reaction stirred at room temperature for 16 hours. During this time the reaction turned from cloudy to clear, and had gone to completion as monitored by LCMS. The reaction was quenched with water; extracted with EtOAc (4×). Combined organic extracts dried over Na₂SO₄, filtered, and concentrated in vacuo. The crude residue was purified by reverse phase chromatography (acetonitrile 40-80%/0.10% NH₄OH in water) to give tert-butyl 8-(8-chloro-2-(pyridin-4-yl)pyrido[3,4-d]pyrimidin-4-yl)-2,8-diazaspiro[4.5]decane-2-carboxylate (192 mg, 0.399 mmol, 52% Yield) as white solid. ¹H NMR (400 MHz, DMSO) δ 8.82-8.77 (m, 2H), 8.39-8.32 (m, 3H), 7.90 (d, J=5.7 Hz, 1H), 3.98 (d, J=6.1 Hz, 2H), 3.39-3.33 (m, 2H), 3.22 (d, J=3.8 Hz, 2H), 1.87-1.82 (m, 2H), 1.84-1.67 (m, 6H), 1.41 (s, 9H). LCMS (ESI) m/z: 481.2 [M+H]⁺.

### Step 2: 8-chloro-2-(pyridin-4-yl)-4-(2,8-diazaspiro[4.5]decan-8-yl)pyrido[3,4-d]pyrimidine trifluoro acetate

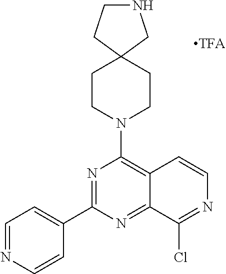 [SMILES: Clc1nccc2c(N3CCC4(CCNC4)CC3)nc(-c3ccncc3)nc12]

To a 1-dram vial was added tert-butyl 8-(8-chloro-2-(pyridin-4-yl)pyrido[3,4-d]pyrimidin-4-yl)-2,8-diazaspiro[4.5]decane-2-carboxylate (192 mg, 0.399 mmol, 1 equiv), followed by DCM (1 mL) and TFA (1 mL). Let stir for 1 h at room temperature. Concentrated in vacuo; concentrated again from DCM. No further purification was required and the residue was lyophilized to furnish 8-chloro-2-(pyridin-4-yl)-4-(2,8-diazaspiro[4.5]decan-8-yl)pyrido[3,4-d]pyrimidine trifluoro acetate. ¹H NMR (400 MHz, DMSO) δ 8.87-8.84 (m, 2H), 8.46-8.42 (m, 2H), 8.40 (d, J=5.6 Hz, 1H), 7.91 (d, J=5.7 Hz, 1H), 4.08-3.94 (m, 4H), 3.36-3.26 (m, 2H), 3.12 (t, J=5.8 Hz, 2H), 1.94 (t, J=7.5 Hz, 2H), 1.87-1.73 (m, 4H) exchangeable NH proton not observed. LCMS (ESI) m/z: 381.1 [M+H]⁺.

### Example 273

### 5-methoxy-2-(3-methyl-1H-pyrazol-4-yl)-4-(2-(oxetan-3-ylmethyl)-2,8-diazaspiro[4.5]decan-8-yl)pyrido[3,4-d]pyrimidine (Compound 273)

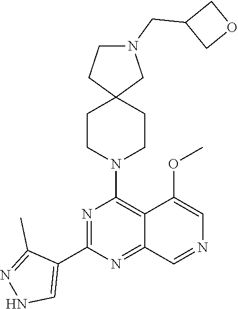 [SMILES: COc1cncc2nc(-c3c[nH]nc3C)nc(N3CCC4(CCN(CC5COC5)C4)CC3)c12]

### Step 1: 5-methoxy-2-(3-methyl-1H-pyrazol-4-yl)-4-(2,8-diazaspiro[4.5]decan-8-yl)pyrido[3,4-d]pyrimidine trifluoroacetate

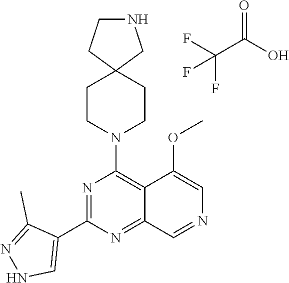

Following the procedure described in Example 142, step 3 and making non-critical variations as required to replace 2-(3-methyl-1-((2-(trimethylsilyl)ethoxy)methyl)-1H-pyrazol-4-yl)pyrido[3,4-d]pyrimidin-4-ol with 5-methoxy-2-(3-methyl-1-((2-(trimethylsilyl)ethoxy)methyl)-1H-pyrazol-4-yl)pyrido[3,4-d]pyrimidin-4-ol, the title compound was obtained (35 mg, crude) as yellow oil. LCMS (ESI) m/z: 380.2 [M+H]⁺.

### Step 2: 5-methoxy-2-(3-methyl-1H-pyrazol-4-yl)-4-(2-(oxetan-3-ylmethyl)-2,8-diazaspiro[4.5]decan-8-yl)pyrido[3,4-d]pyrimidine

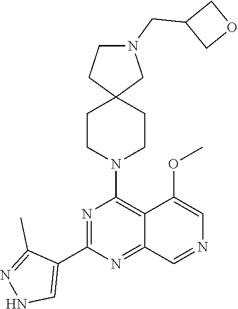 [SMILES: COc1cncc2nc(-c3c[nH]nc3C)nc(N3CCC4(CCN(CC5COC5)C4)CC3)c12]

To a solution of NaBH(OAc)₃ (59 mg, 0.28 mmol), 5-methoxy-2-(3-methyl-1H-pyrazol-4-yl)-4-(2,8-diazaspiro[4.5]decan-8-yl)pyrido[3,4-d]pyrimidine (35 mg, 0.09 mmol) in MeOH (2 mL) was added oxetane-3-carbaldehyde (16 mg, 0.18 mmol). The reaction was stirred at room temperature for 16 h. The mixture was concentrated in vacuo. The crude residue was purified by reverse phase chromatography (acetonitrile 25-55%/0.05% NH₃·H₂O+10 mM NH₄HCO₃ in water) to give the title compound (2 mg, 5%) as a yellow solid. ¹H NMR (400 MHz, DMSO-d₆) δ 12.83 (s, 1H), 8.64 (s, 1H), 8.16 (s, 1H), 8.07 (s, 1H), 4.67-4.59 (m, 2H), 4.30-4.22 (m, 2H), 4.02 (s, 3H), 3.81-3.50 (m, 4H), 3.23-3.01 (m, 1H), 2.70-2.66 (m, 4H), 2.63 (s, 2H), 2.37 (s, 3H), 1.68-1.57 (m, 6H). LCMS (ESI) m/z: 450.1 [M+H]⁺.

### Example 274

### 5-(difluoromethoxy)-2-(pyridin-4-yl)-4-(2,8-diazaspiro[4.5]decan-8-yl)pyrido[3,4-d]pyrimidine (Compound 274)

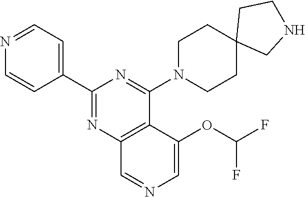 [SMILES: FC(F)Oc1cncc2nc(-c3ccncc3)nc(N3CCC4(CCNC4)CC3)c12]

### 5-(difluoromethoxy)-2-(pyridin-4-yl)-4-(2,8-diazaspiro[4.5]decan-8-yl)pyrido[3,4-d]pyrimidine

### Step 1: tert-butyl 8-(5-(difluoromethoxy)-2-(pyridin-4-yl)pyrido[3,4-d]pyrimidin-4-yl)-2,8-diazaspiro[4.5]decane-2-carboxylate

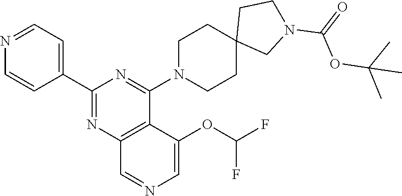 [SMILES: CC(C)(C)OC(=O)N1CCC2(CCN(c3nc(-c4ccncc4)nc4cncc(OC(F)F)c34)CC2)C1]

A solution of tert-butyl 8-(5-hydroxy-2-(pyridin-4-yl)pyrido[3,4-d]pyrimidin-4-yl)-2,8-diazaspiro[4.5]decane-2-carboxylate (200 mg, 0.43 mmol), Cs₂CO₃ (400 mg, 1.23 mmol) and sodium 2-chloro-2,2-difluoroacetate (200 mg, 1.31 mmol) in DMF (5 mL) and water (0.5 mL) was stirred at 80° C. for 6 h. After cooling to room temperature, the reaction mixture was diluted with EtOAc (40 mL), washed with water (30 mL×2). The organic layer was dried over anhydrous Na₂SO₄, filtered and concentrated in vacuo. The residue was purified by silica gel chromatography (solvent gradient: 0-50% EtOAc in petroleum ether) to give the title compound (40 mg, 18%) as a yellow oil. LCMS (ESI) m/z: 513.1 [M+H]⁺.

### Step 2: 5-(difluoromethoxy)-2-(pyridin-4-yl)-4-(2,8-diazaspiro[4.5]decan-8-yl)pyrido[3,4-d]pyrimidine

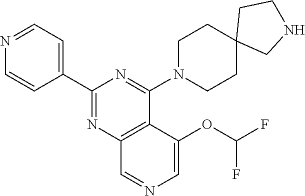 [SMILES: FC(F)Oc1cncc2nc(-c3ccncc3)nc(N3CCC4(CCNC4)CC3)c12]

A solution of tert-butyl 8-(5-(difluoromethoxy)-2-(pyridin-4-yl)pyrido[3,4-d]pyrimidin-4-yl)-2,8-diazaspiro[4.5]decane-2-carboxylate (40 mg, 0.08 mmol) in DCM (2 mL) and trifluoroacetic acid (1 mL) was stirred at room temperature for 16 h. The mixture was concentrated in vacuo. The crude residue was purified by reverse phase chromatography (acetonitrile 30-60%/0.05% NH₃·H₂O+10 mM NH₄HCO₃ in water) to give the title compound (7 mg, 21%) as a white solid. ¹H NMR (400 MHz, DMSO-d₆) δ 9.14 (s, 1H), 8.79-8.77 (m, 2H), 8.45 (s, 1H), 8.34-8.31 (m, 2H), 7.36 (t, J=72.8 Hz, 1H), 3.84-3.61 (m, 4H), 3.20 (s, 1H), 3.00-2.93 (m, 1H), 2.77 (s, 1H), 2.62-2.56 (m, 1H), 1.84-1.78 (m, 1H), 1.70-1.65 (m, 5H). LCMS (ESI) m/z: 413.0 [M+H].

### Example 275

### 8-chloro-5-methoxy-4-(2-methyl-2,8-diazaspiro[4.5]decan-8-yl)-2-(pyridin-4-yl)pyrido[3,4-d]pyrimidine (Compound 275)

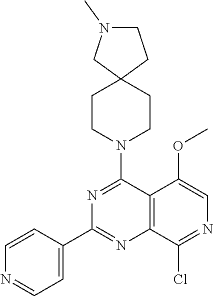 [SMILES: COc1cnc(Cl)c2nc(-c3ccncc3)nc(N3CCC4(CCN(C)C4)CC3)c12]

### Step 1: 2-chloro-3-fluoro-5-methoxyisonicotinic acid

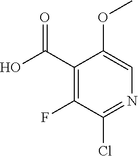

To a solution of 2-chloro-3-fluoro-5-methoxy-pyridine (0.9 g, 5.57 mmol) (prepared according to the procedure in WO202047447) in THF (20 mL) was added n-butyllithium (3.34 mL, 8.36 mmol) at −78° C. After stirring at the same temperature for 0.5 h, solid carbon dioxide (2.45 g, 55.71 mmol) in THF (20 mL) was added at −78° C. The reaction mixture was stirred at room temperature for 2 h under nitrogen atmosphere. The reaction was quenched with sat. aq. NH₄Cl (50 mL) and extracted with EtOAc (30 mL×3). The combined organic layers were washed with brine (30 mL), dried over anhydrous Na₂SO₄, filtered and concentrated in vacuo. The crude residue was purified by silica gel chromatography (solvent gradient: 0-30% EtOAc in petroleum ether) to give the title compound (450 mg, 29%) as a yellow solid. LCMS (ESI) m/z: 205.7 [M+H]⁺.

### Step 2: 8-chloro-5-methoxy-2-(pyridin-4-yl)pyrido[3,4-d]pyrimidin-4-ol

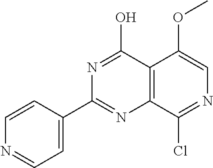 [SMILES: COc1cnc(Cl)c2nc(-c3ccncc3)nc(O)c12]

Following the procedure described in Example 250, step 1-2 and making non-critical variations as required to replace 3,5-difluoroisonicotinic acid with 2-chloro-3-fluoro-5-methoxyisonicotinic acid, the title compound was obtained (55 mg, 70%) as a white solid. LCMS (ESI) m/z: 288.6 [M+H]⁺.

### Step 3: 8-chloro-5-methoxy-4-(2-methyl-2,8-diazaspiro[4.5]decan-8-yl)-2-(pyridin-4-yl)pyrido[3,4-d]pyrimidine

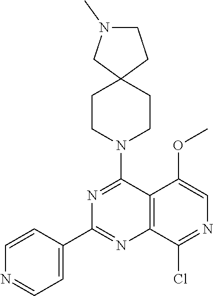 [SMILES: COc1cnc(Cl)c2nc(-c3ccncc3)nc(N3CCC4(CCN(C)C4)CC3)c12]

Following the procedure described in Example 209, step 3 and making non-critical variations as required to replace 2-(pyridin-4-yl)-6-(trifluoromethyl)pyrido[3,4-d]pyrimidin-4-ol and tert-butyl 2,8-diazaspiro[4.5]decane-2-carboxylate with 8-chloro-5-methoxy-2-(pyridin-4-yl)pyrido[3,4-d]pyrimidin-4-ol and 2-methyl-2,8-diazaspiro[4.5]decane, the title compound was obtained (12 mg, 15%) as a yellow solid. ¹H NMR (400 MHz, DMSO-d₆) δ 8.80-8.76 (m, 2H), 8.33-8.29 (m, 2H), 8.10 (s, 1H), 4.06 (s, 3H), 3.80-3.60 (m, 4H), 2.51-2.47 (m, 2H), 2.36 (s, 2H), 2.22 (s, 3H), 1.65 (m, 6H). LCMS (ESI) m/z: 425.2[M+H]⁺.

### Example 276

### 5-methoxy-4-(3-(methoxymethyl)-2,8-diazaspiro[4.5]decan-8-yl)-2-(pyridin-4-yl)pyrido[3,4-d]pyrimidine (Compound 276)

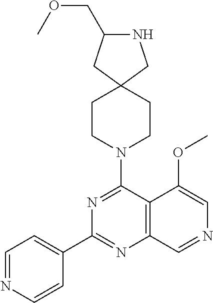 [SMILES: COCC1CC2(CCN(c3nc(-c4ccncc4)nc4cncc(OC)c34)CC2)CN1]

### Step 1: benzyl 8-(5-methoxy-2-(pyridin-4-yl)pyrido[3,4-d]pyrimidin-4-yl)-3-(methoxymethyl)-2,8-diazaspiro[4.5]decane-2-carboxylate

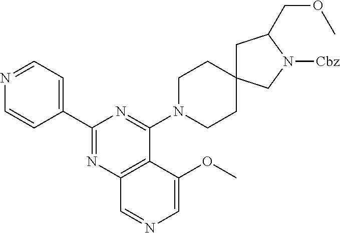 [SMILES: COCC1CC2(CCN(c3nc(-c4ccncc4)nc4cncc(OC)c34)CC2)CN1C(=O)OCc1ccccc1]

Following the procedure described in Example 250, Step 4 and making non-critical variations as required to replace tert-butyl 2,8-diazaspiro[4.5]decane-2-carboxylate with benzyl 3-(methoxymethyl)-2,8-diazaspiro[4.5]decane-2-carboxylate hydrochloride, the title compound was obtained as a yellow solid. LCMS (ESI) m/z: 555.0 [M+H]⁺.

### Step 2: 5-methoxy-4-(3-(methoxymethyl)-2,8-diazaspiro[4.5]decan-8-yl)-2-(pyri din-4-yl)pyrido[3,4-d]pyrimidine

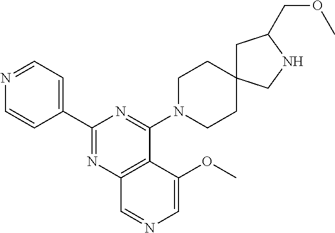 [SMILES: COCC1CC2(CCN(c3nc(-c4ccncc4)nc4cncc(OC)c34)CC2)CN1]

A solution of benzyl 8-(5-methoxy-2-(pyridin-4-yl)pyrido[3,4-d]pyrimidin-4-yl)-3-(methoxymethyl)-2,8-diazaspiro[4.5]decane-2-carboxylate (70 mg, 0.13 mmol) in trifluoroacetic acid (3 mL, 39 mmol) was heated to 60° C. for 16 h. After cooling to room temperature, the mixture was concentrated in vacuo. The residue was adjusted to pH 9 with NH₃·H₂O and the mixture was purified by reverse phase chromatography (acetonitrile 25-55%/0.05% NH₃·H₂O+10 mM NH₄HCO₃ in water) to give the title compound (2 mg, 4%) as a white solid. ¹H NMR (400 MHz, DMSO-d₆) δ 8.82 (s, 1H), 8.77-8.74 (m, 2H), 8.33 (s, 1H), 8.31-8.27 (m, 2H), 4.07 (s, 3H), 3.78-3.60 (m, 4H), 3.29-3.26 (m, 2H), 3.25 (s, 3H), 3.23-3.19 (m, 1H), 2.71 (s, 2H), 1.84-1.76 (m, 1H), 1.76-1.51 (m, 5H). LCMS (ESI) m/z: 421.0 [M+H]⁺.

### Example 277

### 3-((4-(2-Methyl-2,8-diazaspiro[4.5]decan-8-yl)-2-(pyridin-4-yl)pyrido[3,4-d]pyrimidin-5-yl)oxy)cyclobutane-1-carbonitrile (Compound 277)

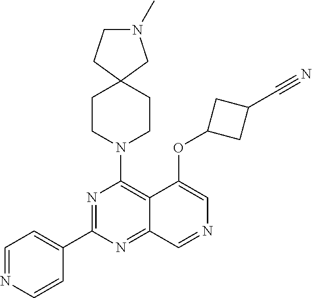 [SMILES: CN1CCC2(CCN(c3nc(-c4ccncc4)nc4cncc(OC5CC(C#N)C5)c34)CC2)C1]

Following the procedure described in Example 260, Step 2 and making non-critical variations as required to replace the alcohol with 3-hydroxycyclobutane-1-carbonitrile was obtained the title compound (15.7 mg, 52% yield) as a mixture of diastereomers. ¹H NMR (400 MHz, DMSO-d6, major isomer) δ 8.83 (d, J=1.4 Hz, 1H), 8.78-8.73 (m, 2H), 8.31-8.27 (m, 2H), 8.08 (s, 1H), 4.98 (p, J=6.9 Hz, 1H), 3.80-3.65 (m, 4H), 3.15 (p, J=8.7 Hz, 1H), 3.10-3.02 (m, 1H), 2.99-2.90 (m, 1H), 2.73-2.64 (m, 1H), 2.60-2.53 (m, 2H), 2.52-2.48 (m, 1H), 2.38 (s, 2H), 2.22 (s, 3H), 1.73-1.58 (m, 6H). LCMS (ESI) m/z: 456.1 [M+H]⁺.

### Example 278

### 1-(3-((4-(2-Methyl-2,8-diazaspiro[4.5]decan-8-yl)-2-(pyridin-4-yl)pyrido[3,4-d]pyrimidin-5-yl)oxy)azetidin-1-yl)ethan-1-one (Compound 278)

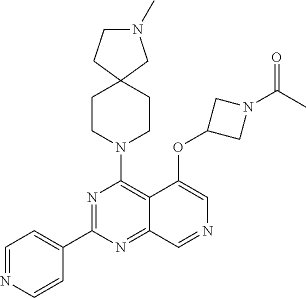 [SMILES: CC(=O)N1CC(Oc2cncc3nc(-c4ccncc4)nc(N4CCC5(CCN(C)C5)CC4)c23)C1]

Following the procedure described in Example 260, Step 2 and making non-critical variations as required to replace the alcohol with 1-(3-hydroxyazetidin-1-yl)ethan-1-one was obtained the title compound as a white solid (16.2 mg, 52% yield). ¹H NMR (400 MHz, DMSO-d₆) δ 8.86 (s, 1H), 8.77-8.74 (m, 2H), 8.31-8.27 (m, 2H), 8.02 (s, 1H), 5.31 (tt, J=6.8, 3.7 Hz, 1H), 4.67 (dd, J=9.8, 6.5 Hz, 1H), 4.39 (dd, J=10.9, 6.3 Hz, 1H), 4.27 (dd, J=9.9, 3.8 Hz, 1H), 3.99 (dd, J=10.7, 3.5 Hz, 1H), 3.82-3.65 (m, 4H), 2.47 (t, J=6.9 Hz, 2H), 2.36 (s, 2H), 2.22 (s, 3H), 1.82 (s, 3H), 1.70-1.57 (m, 6H). LCMS (ESI) m/z: 474.2 [M+H]⁺.

### Example 279

### 8-(5-methoxy-2-(pyridin-4-yl)pyrido[3,4-d]pyrimidin-4-yl)-2,8-diazaspiro[4.5]decan-1-one (Compound 279)

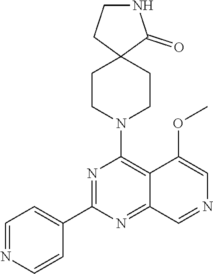 [SMILES: COc1cncc2nc(-c3ccncc3)nc(N3CCC4(CCNC4=O)CC3)c12]

Following the procedure described in Example 250, Step 4 and making non-critical variations as required to replace tert-butyl 2,8-diazaspiro[4.5]decane-2-carboxylate with 2,8-diazaspiro[4.5]decan-1-one (prepared according to the procedure in WO201418764), the title compound was obtained (12 mg, 8%) as a white solid. ¹H NMR (400 MHz, DMSO-d₆) δ 8.84 (s, 1H), 8.76 (d, J=5.6 Hz, 2H), 8.35 (s, 1H), 8.31 (d, J=5.6 Hz, 2H), 7.66 (s, 1H), 4.27-4.14 (m, 2H), 4.07 (s, 3H), 3.42-3.34 (m, 2H), 3.22 (t, J=6.8 Hz, 2H), 2.07 (t, J=6.8 Hz, 2H), 1.88-1.77 (m, 2H), 1.58-1.49 (m, 2H), LCMS (ESI) m/z: 391.1 [M+H]⁺.

### Example 280

### 4-(2-(2,2-difluoroethyl)-2,8-diazaspiro[4.5]decan-8-yl)-5-methoxy-2-(pyridin-4-yl)pyrido[3,4-d]pyrimidine (Compound 280)

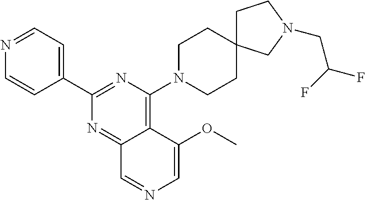 [SMILES: COc1cncc2nc(-c3ccncc3)nc(N3CCC4(CCN(CC(F)F)C4)CC3)c12]

### Step 1: tert-butyl 2-(2,2-difluoroethyl)-2,8-diazaspiro[4.5]decane-8-carboxylate

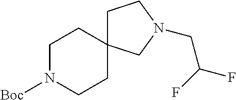 [SMILES: CC(C)(C)OC(=O)N1CCC2(CCN(CC(F)F)C2)CC1]

To a solution of tert-butyl 2,8-diazaspiro[4.5]decane-8-carboxylate (500 mg, 2.08 mmol) in acetonitrile (6 mL) was added 1,1-difluoro-2-10 do-ethane (480 mg, 2.5 mmol) and potassium carbonate (575 mg, 4.2 mmol). The mixture was heated to 70° C. for 16 h. After cooling to room temperature, the reaction mixture was quenched with water (40 mL), extracted with EtOAc (50 mL×2). The combined organic layers were washed with brine (50 mL), dried over anhydrous Na₂SO₄, filtered and concentrated in vacuo to give the title compound (530 mg, 84%) as yellow oil that required no further purification. ¹H NMR (400 MHz, CDCl₃) δ 6.02 (tt, J=56.0, 4.0 Hz, 1H), 3.47-3.26 (m, 4H), 2.87-2.77 (m, 2H), 2.71 (t, J=6.8 Hz, 2H), 2.51 (s, 2H), 1.65 (t, J=6.8 Hz, 2H), 1.55-1.49 (m, 4H), 1.46 (s, 9H). LCMS (ESI) m/z: 305.3 [M+H]⁺.

### Step 2: 2-(2,2-difluoroethyl)-2,8-diazaspiro[4.5]decane hydrochloride

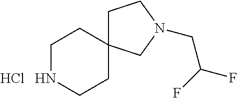 [SMILES: Cl ; FC(F)CN1CCC2(CCNCC2)C1]

To a solution of tert-butyl 2-(2,2-difluoroethyl)-2,8-diazaspiro[4.5]decane-8-carboxylate (260 mg, 0.85 mmol) in dioxane (20 mL) was added 4M HCl in dioxane (2 mL, 8 mmol). The mixture was stirred at room temperature for 1 h. The mixture was concentrated in vacuo to give the title compound (200 mg, crude) as a yellow solid that required no further purification. LCMS (ESI) m/z: 205.2 [M+H]⁺.

### Step 3: 4-(2-(2,2-difluoroethyl)-2,8-diazaspiro[4.5]decan-8-yl)-5-methoxy-2-(pyri din-4-yl)pyrido[3,4-d]pyrimidine

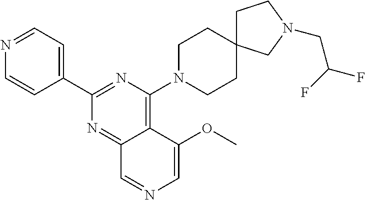 [SMILES: COc1cncc2nc(-c3ccncc3)nc(N3CCC4(CCN(CC(F)F)C4)CC3)c12]

Following the procedure described in Example 250, Step 4 and making non-critical variations as required to replace tert-butyl 2,8-diazaspiro[4.5]decane-2-carboxylate with 2-(2,2-difluoroethyl)-2,8-diazaspiro[4.5]decane hydrochloride, the title compound was obtained (11 mg, 30%) as a white solid. ¹H NMR (400 MHz, DMSO-d6) δ 8.82-8.78 (m, 1H), 8.77-8.72 (m, 2H), 8.33-8.30 (m, 1H), 8.29-8.25 (m, 2H), 6.09 (tt, J=56.0, 4.0 Hz, 1H), 4.06 (s, 3H), 3.76-3.57 (m, 4H), 2.88-2.75 (m, 2H), 2.72-2.62 (m, 2H), 2.54 (s, 2H), 1.77-1.69 (m, 2H), 1.68-1.62 (m, 4H). LCMS (ESI) m/z: 441.2 [M+H]⁺.

### Example 281

### 4-(2-(2,2-difluoroethyl)-2,8-diazaspiro[4.5]decan-8-yl)-5-methoxy-2-(5-methyl-1H-pyrazol-4-yl)pyrido[3,4-d]pyrimidine (Compound 281)

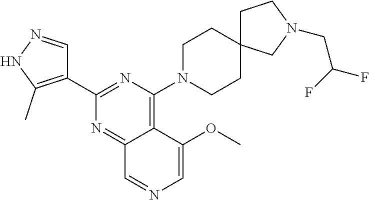 [SMILES: COc1cncc2nc(-c3cn[nH]c3C)nc(N3CCC4(CCN(CC(F)F)C4)CC3)c12]

### Step 1: 4-(2-(2,2-difluoroethyl)-2,8-diazaspiro[4.5]decan-8-yl)-5-methoxy-2-(5-methyl-1-((2-(trimethylsilyl)ethoxy)methyl)-1H-pyrazol-4-yl)pyrido[3,4-d]pyrimidine

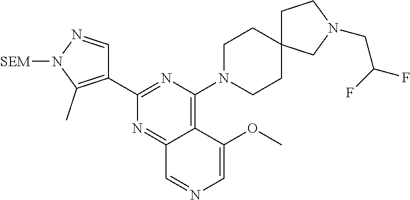 [SMILES: COc1cncc2nc(-c3cnn(COCC[Si](C)(C)C)c3C)nc(N3CCC4(CCN(CC(F)F)C4)CC3)c12]

Following the procedure described in Example 250, Step 4 and making non-critical variations as required to replace 5-methoxy-2-(pyridin-4-yl)pyrido[3,4-d]pyrimidin-4-ol and tert-butyl 2,8-diazaspiro[4.5]decane-2-carboxylate with 5-methoxy-2-(5-methyl-1-((2-(trimethylsilyl)ethoxy)methyl)-1H-pyrazol-4-yl)pyrido[3,4-d]pyrimidin-4-ol and 2-(2,2-difluoroethyl)-2,8-diazaspiro[4.5]decane hydrochloride, the title compound was obtained as a yellow solid. LCMS (ESI) m/z: 574.3 [M+H]⁺.

### Step 2: 4-(2-(2,2-difluoroethyl)-2,8-diazaspiro[4.5]decan-8-yl)-5-methoxy-2-(5-methyl-1H-pyrazol-4-yl)pyrido[3,4-d]pyrimidine

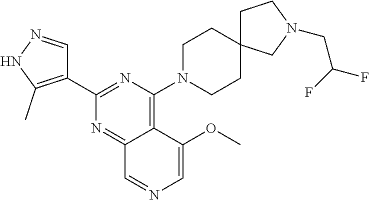 [SMILES: COc1cncc2nc(-c3cn[nH]c3C)nc(N3CCC4(CCN(CC(F)F)C4)CC3)c12]

To a solution of 4-(2-(2,2-difluoroethyl)-2,8-diazaspiro[4.5]decan-8-yl)-5-methoxy-2-(5-methyl-1-((2-(trimethylsilyl)ethoxy)methyl)-1H-pyrazol-4-yl)pyrido[3,4-d]pyrimidine (190 mg, 0.33 mmol) in DCM (4 mL) was added trifluoroacetic acid (2 mL, 26 mmol). The mixture was stirred at room temperature for 2 h. The mixture was concentrated in vacuo and the crude residue was purified by reverse phase chromatography (acetonitrile 37-67%/0.05% NH₃·H₂O+10 mM NH₄HCO₃ in water) to give the title compound (9 mg, 6%) as a yellow solid. ¹H NMR (400 MHz, DMSO-d₆) δ 12.85 (s, 1H), 8.64 (s, 1H), 8.16 (s, 1H), 8.05 (s, 1H), 6.08 (tt, J=56.0, 4.0 Hz, 1H), 4.02 (s, 3H), 3.59-3.47 (m, 4H), 2.85-2.79 (m, 2H), 2.68-2.63 (m, 4H), 2.52 (s, 3H), 1.68-1.59 (m, 6H). LCMS (ESI) m/z: 444.3 [M+H]⁺.

### Example 282

### 4-[2-[(3-Fluorooxetan-3-yl)methyl]-2,8-diazaspiro[4.5]decan-8-yl]-5-methoxy-2-(4-pyridyl)pyrido[3,4-d]pyrimidine (Compound 282)

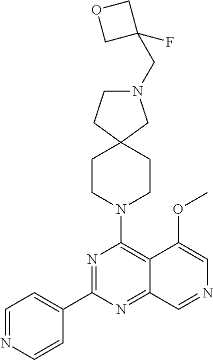 [SMILES: COc1cncc2nc(-c3ccncc3)nc(N3CCC4(CCN(CC5(F)COC5)C4)CC3)c12]

To a solution of 5-methoxy-2-(pyridin-4-yl)-4-(2,8-diazaspiro[4.5]decan-8-yl)pyrido[3,4-d]pyrimidine (50 mg, 0.133 mmol) in anhydrous 1,4-dioxane (0.66 mL, 0.2 M) was added (3-fluorooxetan-3-yl)methyl 4-methylbenzenesulfonate (41.49 g, 0.159 mmol, 1.2 equiv) followed by N,N-diisopropylethylamine (0.116 mL, 0.664 mmol, 5 equiv) under N₂ atmosphere. The resulting mixture was stirred at 100° C. for 3 days, then cooled to room temperature. The volatiles were removed under reduced pressure, and the crude residue was purified by HPLC (Triart C18 (50×30 mm, 5 pm), 0.1% NH₄OH in H₂O/MeCN 20-60% gradient, 60 mL/min), followed by SFC (ChiralART SJ (150×21.2 mm, 5 pm), 0.1% NH₄OH in MeOH 35% isocratic, 70 mL/min). The title product was obtained as a white solid (3.31 mg, 0.0071 mmol, yield=5%). ¹H NMR (400 MHz, DMSO-d₆) δ 8.83 (s, 1H), 8.79-8.73 (m, 2H), 8.33 (s, 1H), 8.32-8.27 (m, 2H), 4.66-4.50 (m, 4H), 4.07 (s, 3H), 3.80-3.56 (m, 4H), 2.94 (d, J=25.4 Hz, 2H), 2.66 (t, J=6.9 Hz, 2H), 2.53 (s, 2H), 1.78-1.57 (m, 6H). LCMS (ESI) m/z: 465.2 [M+H]⁺.

### Example 283

### 1,1,1-Trifluoro-3-[8-[5-methoxy-2-(4-pyridyl)pyrido[3,4-d]pyrimidin-4-yl]-2,8-diazaspiro[4.5]decan-2-yl]-2-methyl-propan-2-ol (Compound 283)

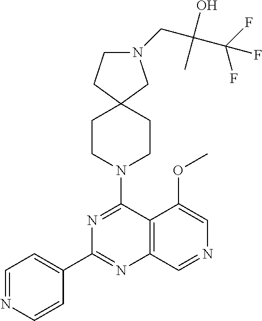 [SMILES: COc1cncc2nc(-c3ccncc3)nc(N3CCC4(CCN(CC(C)(O)C(F)(F)F)C4)CC3)c12]

To a 2-dram vial containing a solution of 5-methoxy-2-(pyridin-4-yl)-4-(2,8-diazaspiro[4.5]decan-8-yl)pyrido[3,4-d]pyrimidine (50 mg, 0.133 mmol) in anhydrous MeOH (0.66 mL, 0.2 M) was added 2-(1,1-difluoroethyl)-2-methyl-oxirane (81.1 mg, 0.664 mmol, 5 equiv), followed by N,N-diisopropylethylamine (0.116 mL, 0.664 mmol, 5 equiv) under N₂ atmosphere. The vial was sealed and the reaction mixture stirred at 100° C. for 30 min, then cooled to room temperature. The volatiles were removed under reduced pressure, and the crude residue was purified by HPLC (XSelect CSH Prep C18 (50×30 mm, 5 pm), 0.1% NH₄OH in H₂O/DMSO 30-70% gradient, 60 mL/min) to afford the title product as a light brown solid (4.1 mg, 0.0082 mmol, 90% purity, yield=6%). ¹H NMR (400 MHz, DMSO-d₆) δ 8.83 (s, 1H), 8.79-8.73 (m, 2H), 8.33 (s, 1H), 8.32-8.28 (m, 2H), 5.69 (s, 1H), 4.07 (s, 3H), 3.78-3.54 (m, 4H), 2.82-2.62 (m, 4H), 2.52 (s, 2H), 1.77-1.59 (m, 6H), 1.30 (s, 3H). LCMS (ESI) m/z: 503.2 [M+H]⁺.

### Example 284

### 4-(2-Methyl-2,8-diazaspiro[4.5]decan-8-yl)-2-(pyridin-4-yl)pyrido[3,4-d]pyrimidine-8-carbonitrile (Compound 284)

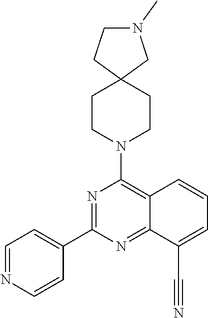 [SMILES: CN1CCC2(CCN(c3nc(-c4ccncc4)nc4c(C#N)cccc34)CC2)C1]

A mixture of 8-chloro-4-(2-methyl-2,8-diazaspiro[4.5]decan-8-yl)-2-(pyridin-4-yl)pyrido[3,4-d]pyrimidine (100 mg, 0.25 mmol), potassium hexacyanoferrate(II) trihydrate (46.6 mg, 0.126 mmol), (2-dicyclohexylphosphino-2′,4′,6′-triisopropyl-1,1′-biphenyl)[2-(2′-amino-1,1′-biphenyl)]palladium(II) methanesulfonate (21.8 mg, 0.025 mmol), potassium acetate (24.9 mg, 0.25 mmol) in 1,4-dioxane (0.25 mL) and water (0.25 mL) under nitrogen atmosphere was heated at 90° C. for 24 h. After cooling down the reaction, DCM (3 mL) and water (3 mL) were added, and insoluble materials were filtered off. The layers were separated, and the aqueous layer was further extracted with DCM (3×3 mL). The combined organic extracts dried over Na₂SO₄, filtered, and concentrated in vacuo. The crude residue was purified by silica gel chromatography (solvent gradient: 0-10% MeOH in DCM) and then by HPLC to give the title compound as white solid (13.2 mg, 14% yield). ¹H NMR (400 MHz, DMSO-d₆) δ 8.85-8.79 (m, 2H), 8.69 (d, J=5.5 Hz, 1H), 8.39-8.31 (m, 2H), 8.23 (d, J=5.5 Hz, 1H), 4.07 (ddd, J=13.6, 6.9, 4.0 Hz, 2H), 3.97 (ddd, J=13.6, 7.6, 3.9 Hz, 2H), 2.55-2.48 (m, 2H), 2.40 (s, 2H), 2.24 (s, 3H), 1.84-1.66 (m, 6H). LCMS (ESI) m/z: 386.2 [M+H].

### Example 285

### 8-chloro-5-methoxy-2-(5-methyl-1H-pyrazol-4-yl)-4-(2,8-diazaspiro[4.5]decan-8-yl)pyrido[3,4-d]pyrimidine (Compound 285)

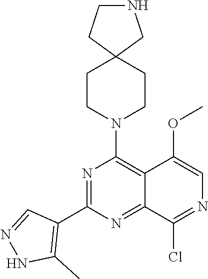 [SMILES: COc1cnc(Cl)c2nc(-c3cn[nH]c3C)nc(N3CCC4(CCNC4)CC3)c12]

### Step 1: methyl 5-methyl-1-(tetrahydro-2H-pyran-2-yl)-1H-pyrazole-4-carboxylate

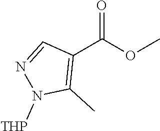

A mixture of methyl 5-methyl-1H-pyrazole-4-carboxylate (2 g, 14.3 mmol), 3,4-dihydro-2H-pyran (2.6 mL, 28.5 mmol) and TsOH (490 mg, 2.9 mmol) in THF (60 mL) was heated to 75° C. for 16 h. After cooling to room temperature, the residue was dissolved in DCM (200 mL), washed with sat. aq. NaHCO₃ (50 mL) and brine (50 mL). The organic layer was dried over anhydrous Na₂SO₄, filtered and concentrated in vacuo to give the title compound (2.7 g, crude) as yellow oil that required no further purification. LCMS (ESI) m/z: 225.2 [M+H].

### Step 2: 5-methyl-1-(tetrahydro-2H-pyran-2-yl)-1H-pyrazole-4-carboximidamide

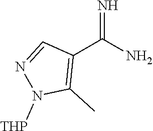

To a solution of NH₄Cl (4.78 g, 89.4 mmol) in toluene (60 mL) was added trimethylaluminum (45 mL, 90 mmol) (2M in toluene) dropwise at 0° C. under nitrogen atmosphere. The mixture was warmed to room temperature and stirred for 3 h. A solution of methyl 5-methyl-1-(tetrahydro-2H-pyran-2-yl)-1H-pyrazole-4-carboxylate (2 g, 8.9 mmol) in toluene (10 mL) was added to the reaction mixture. The resulting mixture was heated to 80° C. for 16 h. After cooling to room temperature, the reaction was quenched slowly with MeOH (50 mL). The resulting white precipitate was removed by filtration on a celite pad. The filtrate was concentrated in vacuo. To this crude residue was added MeOH (10 mL) and methyl tert-butyl ether (30 mL), and then filtered. The filtrate was concentrated in vacuo to give the title compound (1.5 g, 81%) as a yellow solid. LCMS (ESI) m/z: 209.2 [M+H]⁺.

### Step 3: 8-chloro-5-methoxy-2-(5-methyl-1-(tetrahydro-2H-pyran-2-yl)-1H-pyrazol-4-yl)pyrido[3,4-d]pyrimidin-4-ol

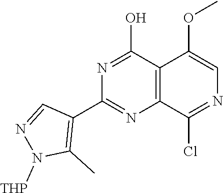

Following the procedure described in Example 250, step 1-2 and making non-critical variations as required to replace 3,5-difluoroisonicotinic acid and isonicotinimidamide hydrochloride with 2-chloro-3-fluoro-5-methoxyisonicotinic acid and 5-methyl-1-(tetrahydro-2H-pyran-2-yl)-1H-pyrazole-4-carboximidamide, the title compound was obtained (85 mg, 60%) as a white solid. LCMS (ESI) m/z: 376.0 [M+H]⁺.

### Step 4: 8-chloro-5-methoxy-2-(5-methyl-1H-pyrazol-4-yl)-4-(2,8-diazaspiro[4.5]decan-8-yl)pyrido[3,4-d]pyrimidine

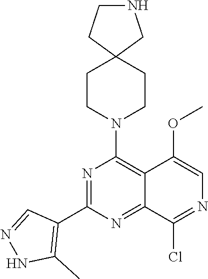 [SMILES: COc1cnc(Cl)c2nc(-c3cn[nH]c3C)nc(N3CCC4(CCNC4)CC3)c12]

Following the procedure described in Example 250, step 4-5 and making non-critical variations as required to replace 5-methoxy-2-(pyridin-4-yl)pyrido[3,4-d]pyrimidin-4-ol with 8-chloro-5-methoxy-2-(5-methyl-1-(tetrahydro-2H-pyran-2-yl)-1H-pyrazol-4-yl)pyrido[3,4-d]pyrimidin-4-ol, the title compound was obtained (3 mg, 4%) as a white solid. ¹H NMR (400 MHz, CD₃OD) δ 6 8.22 (s, 1H), 7.88 (s, 1H), 4.08 (s, 3H), 3.83-3.61 (m, 4H), 3.19 (t, J=7.6 Hz, 2H), 2.97 (s, 2H), 2.77 (s, 3H), 1.89 (t, J=7.2 Hz, 2H), 1.82-1.74 (m, 4H). Pyrazole NH and amine NH protons not observed. LCMS (ESI) m/z: 414.0 [M+H]⁺.

### Example 286

### 8-chloro-5-methoxy-2-(5-methyl-1H-pyrazol-4-yl)-4-(2-methyl-2,8-diazaspiro[4.5]decan-8-yl)pyrido[3,4-d]pyrimidine (Compound 286)

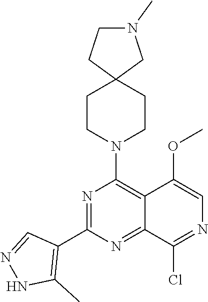 [SMILES: COc1cnc(Cl)c2nc(-c3cn[nH]c3C)nc(N3CCC4(CCN(C)C4)CC3)c12]

Following the procedure described in Example 102 and making non-critical variations as required to replace 4-(2,8-diazaspiro[4.5]decan-8-yl)-2-(4-pyridyl)pyrido[3,4-d]pyrimidine hydrochloride with 8-chloro-5-methoxy-2-(5-methyl-1H-pyrazol-4-yl)-4-(2,8-diazaspiro[4.5]decan-8-yl)pyrido[3,4-d]pyrimidine, the title compound was obtained (12 mg, 23%) as a white solid. ¹H NMR (400 MHz, DMSO-d₆) δ 12.92 (s, 1H), 8.04 (s, 1H), 7.94 (s, 1H), 4.02 (s, 3H), 3.68-3.50 (m, 4H), 2.67 (s, 3H), 2.49-2.45 (m, 2H), 2.35 (s, 2H), 2.22 (s, 3H), 1.66-1.58 (m, 6H). LCMS (ESI) m/z: 428.0 [M+H]⁺.

### Example 287

### 1-(8-(8-chloro-5-methoxy-2-(pyridin-4-yl)pyrido[3,4-d]pyrimidin-4-yl)-2,8-diazaspiro[4.5]decan-2-yl)-2-methylpropan-2-ol (Compound 287)

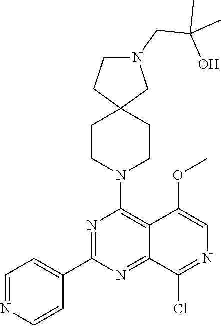 [SMILES: COc1cnc(Cl)c2nc(-c3ccncc3)nc(N3CCC4(CCN(CC(C)(C)O)C4)CC3)c12]

Following the procedure described in Example 250, step 4-6 and making non-critical variations as required to replace 5-methoxy-2-(pyridin-4-yl)pyrido[3,4-d]pyrimidin-4-ol with 8-chloro-5-methoxy-2-(pyridin-4-yl)pyrido[3,4-d]pyrimidin-4-ol, the title compound was obtained (33 mg, 31%) as a yellow solid. ¹H NMR (400 MHz, DMSO-d₆) δ 8.80-8.75 (m, 2H), 8.33-8.27 (m, 2H), 8.10 (s, 1H), 4.06 (s, 3H), 4.05-4.02 (m, 1H), 3.77-3.60 (m, 4H), 2.66 (t, J=6.8 Hz, 2H), 2.52 (s, 2H), 2.32 (s, 2H), 1.69-1.58 (m, 6H), 1.08 (s, 6H). LCMS (ESI) m/z: 483.3 [M+H]⁺.

### Example 288

### 8-chloro-4-(2-(2,2-difluoroethyl)-2,8-diazaspiro[4.5]decan-8-yl)-2-(pyridin-4-yl)pyrido[3,4-d]pyrimidine (Compound 288)

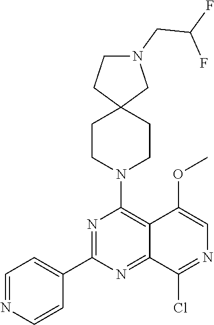 [SMILES: COc1cnc(Cl)c2nc(-c3ccncc3)nc(N3CCC4(CCN(CC(F)F)C4)CC3)c12]

Following the procedure described in Example 250, Step 4 and making non-critical variations as required to replace 5-methoxy-2-(pyridin-4-yl)pyrido[3,4-d]pyrimidin-4-ol and tert-butyl 2,8-diazaspiro[4.5]decane-2-carboxylate with 8-chloro-2-(pyridin-4-yl)pyrido[3,4-d]pyrimidin-4-ol (prepared according to the procedure in WO201452699) and 2-(2,2-difluoroethyl)-2,8-diazaspiro[4.5]decane hydrochloride, the title compound was obtained as a yellow solid. ¹H NMR (400 MHz, DMSO-d₆) δ 8.81-8.77 (m, 2H), 8.39-8.28 (m, 3H), 7.88 (d, J=5.6 Hz, 1H), 6.11 (tt, J=56.0, 2.0, 1H), 4.05-3.82 (m, 4H), 2.90-2.77 (m, 2H), 2.74-2.66 (m, 2H), 2.57 (s, 2H), 1.81-1.67 (m, 6H). LCMS (ESI) m/z: 445.3 [M+H]⁺.

### Example 289

### 2-(pyridin-4-yl)-4-(2,8-diazaspiro[4.5]decan-8-yl)-5-(trifluoromethoxy)pyrido[3,4-d]pyrimidine (Compound 289)

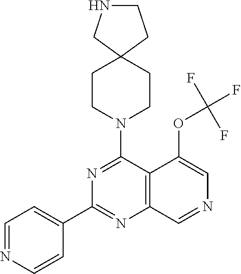 [SMILES: FC(F)(F)Oc1cncc2nc(-c3ccncc3)nc(N3CCC4(CCNC4)CC3)c12]

The title compound is synthesized following a procedure similar to the procedure described in Example 274.

### Example 290

### 8-chloro-4-(2-(2,2-difluoroethyl)-2,8-diazaspiro[4.5]decan-8-yl)-2-(5-methyl-1H-pyrazol-4-yl)pyrido[3,4-d]pyrimidine (Compound 290)

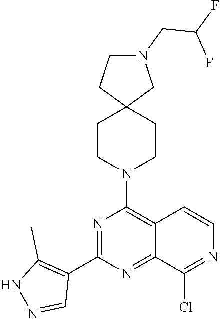 [SMILES: Cc1[nH]ncc1-c1nc(N2CCC3(CCN(CC(F)F)C3)CC2)c2ccnc(Cl)c2n1]

The title compound is synthesized following a procedure similar to the procedure described in Example 107, Step 1 and making non-critical variations as required to replace 4-(2,8-diazaspiro[4.5]decan-8-yl)-2-(4-pyridyl)pyrido[3,4-d]pyrimidine hydrochloride and methyl 2-bromo-2-methylpropanoate with 8-chloro-2-(3-methyl-1H-pyrazol-4-yl)-4-(2,8-diazaspiro[4.5]decan-8-yl)pyrido[3,4-d]pyrimidine and 2,2-difluoroethyl trifluoromethanesulfonate.

### Example 291

### 1-(8-(8-chloro-5-methoxy-2-(5-methyl-1H-pyrazol-4-yl)pyrido[3,4-d]pyrimidin-4-yl)-2,8-diazaspiro[4.5]decan-2-yl)-2-methylpropan-2-ol (Compound 291)

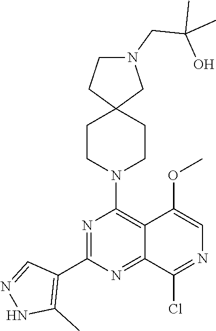 [SMILES: COc1cnc(Cl)c2nc(-c3cn[nH]c3C)nc(N3CCC4(CCN(CC(C)(C)O)C4)CC3)c12]

Following the procedure described in Example 250, step 6 and making non-critical variations as required to replace 5-methoxy-2-(pyridin-4-yl)-4-(2,8-diazaspiro[4.5]decan-8-yl)pyrido[3,4-d]pyrimidine hydrochloride with 8-chloro-5-methoxy-2-(5-methyl-1H-pyrazol-4-yl)-4-(2,8-diazaspiro[4.5]decan-8-yl)pyrido[3,4-d]pyrimidine hydrochloride, the title compound was obtained (2 mg, 2%) as a white solid. ¹H NMR (400 MHz, DMSO-d₆) δ 12.92 (s, 1H), 8.05 (s, 1H), 7.94 (s, 1H), 4.04 (s, 1H), 4.02 (s, 3H), 3.67-3.49 (m, 4H), 2.72-2.63 (m, 4H), 2.53 (s, 3H), 2.32 (s, 2H), 1.68-1.56 (m, 6H), 1.08 (s, 6H). LCMS (ESI) m/z: 486.4 [M+H]⁺.

### Example 292

### 3-(4-(2-methyl-2,8-diazaspiro[4.5]decan-8-yl)-2-(pyridin-4-yl)pyrido[3,4-d]pyrimidin-8-yl)prop-2-yn-1-ol (Compound 292)

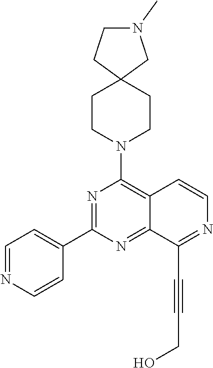 [SMILES: CN1CCC2(CCN(c3nc(-c4ccncc4)nc4c(C#CCO)nccc34)CC2)C1]

### Step 1: 3-(2-(pyridin-4-yl)-4-(2,8-diazaspiro[4.5]decan-8-yl)pyrido[3,4-d]pyrimidin-8-yl)prop-2-yn-1-ol

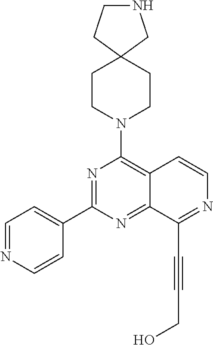 [SMILES: OCC#Cc1nccc2c(N3CCC4(CCNC4)CC3)nc(-c3ccncc3)nc12]

Following the procedure described in Example 245, step 1-2 and making non-critical variations as required to replace 2-methyl-3-butyn-2-ol with prop-2-yn-1-ol, the title compound was obtained (100 mg, crude) as brown oil. LCMS (ESI) m/z: 401.2 [M+H]⁺.

### Step 2: 3-(4-(2-methyl-2,8-diazaspiro[4.5]decan-8-yl)-2-(pyridin-4-yl)pyrido[3,4-d]pyrimidin-8-yl)prop-2-yn-1-ol

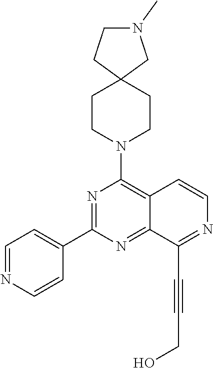 [SMILES: CN1CCC2(CCN(c3nc(-c4ccncc4)nc4c(C#CCO)nccc34)CC2)C1]

Following the procedure described in Example 102 and making non-critical variations as required to replace 4-(2,8-diazaspiro[4.5]decan-8-yl)-2-(4-pyridyl)pyrido[3,4-d]pyrimidine hydrochloride with 3-(2-(pyridin-4-yl)-4-(2,8-diazaspiro[4.5]decan-8-yl)pyrido[3,4-d]pyrimidin-8-yl)prop-2-yn-1-ol, the title compound was obtained (6 mg, 6%) as a yellow solid. ¹H NMR (400 MHz, CD₃OD) δ 8.73-8.69 (m, 2H), 8.57-8.51 (m, 2H), 8.47 (d, J=6.0 Hz, 1H), 7.88 (d, J=5.6 Hz, 1H), 4.63 (s, 2H), 4.16-4.02 (m, 2H), 4.01-3.90 (m, 2H), 2.71 (t, J=6.8 Hz, 2H), 2.59 (s, 2H), 2.40 (s, 3H), 1.92-1.78 (m, 6H). LCMS (ESI) m/z: 415.2 [M+H]⁺.

### BIOLOGICAL EXAMPLES

Exemplary compounds of Formula (I) were tested to assess compound inhibition according to the following protocols.

### Example B1: Standard LATS2 HTRF Assay

Human LATS2 catalytic domain which contains the amino acids G553-V1088 (accession NP_055387) was purified in-house. The LATS2 catalytic domain was co-purified with Mob1b (accession NP_775739). The LATS2 biochemical HTRF assay was performed using the HTRF KinEASE-STK S1 Kit (Cisbio, Cat #62ST1PEC), following the protocol from the manufacturer. Compounds were dispensed by the Echo Liquid Handler (Labcyte) into a white 384-well plate (PerkinElmer, Cat #6008289). 3 uL of 2×LATS2 enzyme solution was added to the compounds, following by a 10 minute incubation at room temperature. Then, 2×ATP and STK S1 peptide solution was added to initiate the one hour enzyme reaction at room temperature. The final condition of the reaction was 0.2 nM LATS2, 50 μM ATP, 0.5 μM STK S1 peptide in 50 mM HEPES pH7.2, 10 mM MgCl₂, 0.1% BGG, 0.005% Brij-35, 1 mM DTT. The reaction was quenched by adding 6 uL of the detection mixture which contained Streptavidin XL665 and STK Antibody-Cryptate (Cisbio), incubate for 1 hour at room temperature. The HTRF (665 nm/620 nm) signal was read on the Envision plater reader (PerkinElmer). The IC₅₀ values were determined by fitting the % inhibition with a nonlinear four-parameter logistical equation. The Kᵢ values were calculated using the Cheng-Prusoff equation for a competitive inhibitor, IC₅₀═Kᵢ (1+S/Kₘ)+M [E] with ATP Kₘ for LATS2=105 μM. The results are set forth in Table B1.

### Example B2: LATS2 High ATP HTRF Assay

The LATS2 high ATP HTRF assay was performed using the same protocol as the Standard LATS2 HTRF assay. The final condition of the reaction was 0.05 nM LATS2, 5000 μM ATP, 0.5 μM STK S1 peptide in 50 mM HEPES pH7.2, 10 mM MgCl₂, 0.1% BGG, 0.005% Brij-35, 1 mM DTT and the 2 hours enzyme reaction at room temperature. The results are set forth in Table B1.

### Example B3: LATS1 High ATP HTRF Assay

Human LATS1 catalytic domain which contains the amino acids E590-V1130 (accession NP_004681) was purified in-house. The LATS1 catalytic domain was co-purified with Mob1b (accession NP_775739). The LATS1 biochemical assay was performed using the HTRF KinEASE-STK S1 Kit (Cisbio, Cat #62ST1PEC), following the protocol from the manufacturer. Compounds were dispensed by the Echo Liquid Handler (Labcyte) into a white 384-well plate (PerkinElmer, Cat #6008289). 3 μL of 2×LATS1 enzyme solution was added to the compounds, followed by a 10 minute incubation at room temperature. Then, 3 μL of 2×ATP and STK S1 peptide solution was added to initiate the enzyme reaction for one hour at room temperature. The final condition of the reaction was 0.025 nM LATS1, 5000 μM ATP, 0.5 μM STK S1 peptide in 50 mM HEPES pH 7.2, 10 mM MgCl₂, 0.1% BGG, 0.005% Brij-35, 1 mM DTT. The reaction was quenched and the HTRF signal was read. The data was analyzed and the Kᵢ values were calculated using the Cheng-Prusoff equation for a competitive inhibitor, corrected for the amount of the enzyme used, IC₅₀═Kᵢ (1+S/Kₘ)+M [E] with ATP Kₘ for LATS1=27 μM. The results are set forth in Table B1.

### Example B4: ROCK1 HTRF Assay

Human Rho associated coiled-coil containing protein kinase 1 (ROCK1) was purchased from Carna Biosciences (Cat #01-109) which contains the catalytic domain (amino acids 1-477 from the accession number NP_005397.1). The ROCK1 biochemical assay was performed using the HTRF KinEASE-STK S2 Kit (Cisbio, Cat #62ST2PEC), following the protocol from the manufacturer. Compounds were dispensed by the Echo Liquid Handler (Labcyte) into a white 384-well plate (PerkinElmer, Cat #6008289). 3 uL of 2×Rockl enzyme solution was added to the compounds, followed by a 10 minute incubation at room temperature. Then, 3 uL 2×ATP and STK S2 peptide solution was added to initiate the enzyme reaction for one hour at room temperature. The final condition of the reaction was 1.5 nM ROCK1, 3 μM ATP, 0.5 μM STK S2 peptide in 50 mM HEPES pH7.2, 10 mM MgCl₂, 0.1% BGG, 0.005% Brij-35, 1 mM DTT. The reaction was quenched by adding 6 uL of the detection mixture which contained Streptavidin XL665 and STK Antibody-Cryptate (Cisbio), incubate for 1 hour at room temperature. The HTRF (665 nm/620 nm) signal was read on the Envision plater reader (PerkinElmer). The IC₅₀ values were determined by fitting the % inhibition with a nonlinear four-parameter logistical equation. The Kᵢ values were calculated using the Cheng-Prusoff equation for a competitive inhibitor, corrected for the amount of the enzyme used, IC₅₀═Kᵢ (1+S/Kₘ)+M [E] with ATP Kₘ for ROCK1=2.8 uM. Compounds of Table 1 showed Kᵢ values for ROCK1 ranging from about 3 nM to >10 μM, and the ratios of ROCK1 Kᵢ to LATS2 Kᵢ of about 2 to over 25,000 folds.

### Example B5: PKA HTRF Assay

Full length human protein kinase A (PKA) (accession NP_002721.1) was purchased from Carna Biosciences (Cat #01-127). The PKA biochemical assay was performed using the HTRF KinEASE-STK S3 Kit (Cisbio, Cat #62ST3PEC), following the protocol from the manufacturer. Compounds were dispensed by the Echo Liquid Handler (Labcyte) into a white 384-well plate (PerkinElmer, Cat #6008289). 3 μL of 2×PKA enzyme solution was added to the compounds, followed by a 10 minute incubation at room temperature. Then, 3 μL of 2×ATP and STK S3 peptide solution was added to initiate the enzyme reaction for one hour at room temperature. The final condition of the reaction was 0.0025 nM PKA, 2.5 μM ATP, 0.5 μM STK S3 peptide in 50 mM HEPES pH 7.2, 10 mM MgCl₂, 0.1% BGG, 0.005% Brij-35, 1 mM DTT. The reaction was quenched and the HTRF signal was read, the data was analyzed, and the Kᵢ values were calculated using the Cheng-Prusoff equation for a competitive inhibitor, corrected for the amount of the enzyme used, IC₅₀═Kᵢ (1+S/Kₘ)+% [E] with ATP Kₘ for PKA=1.9 μM. Compounds of Table 1 showed Kᵢ values for PKA ranging from 3 nM to >1200 nM, and the ratios of PKA Kᵢ to LATS2 Kᵢ of about 1.3 to over 1,000 folds for some of the compounds; and Kᵢ values for PKA ranging from 0.6 nM to >1200 nM, and the ratios of PKA Kᵢ to LATS2 Kᵢ of about 1.3 to over 5,000 folds for other compounds.

### Example B6: AKT1 HTRF Assay

Full length human AKT serine-threonine protein kinase 1 (AKT1) (accession NP_001014431) was purified in-house. The AKT1 biochemical assay was performed using the HTRF KinEASE-STK S3 Kit (Cisbio, Cat #62ST3PEC), following the protocol from the manufacturer. Compounds were dispensed by the Echo Liquid Handler (Labcyte) into a white 384-well plate (PerkinElmer, Cat #6008289). 3 μL of 2×ATK1 enzyme solution was added to the compounds, followed by a 10 minute incubation at room temperature. Then, 3 μL of 2×ATP and STK S3 peptide solution was added to initiate the enzyme reaction for one hour at room temperature. The final condition of the reaction was 3 nM AKT1, 50 μM ATP, 0.5 μM STK S3 peptide in 50 mM HEPES pH 7.2, 10 mM MgCl₂, 0.1% BGG, 0.005% Brij-35, 1 mM DTT. The reaction was quenched and the HTRF signal was read, the data was analyzed, and the Kᵢ values were calculated using the Cheng-Prusoff equation for a competitive inhibitor, corrected for the amount of the enzyme used, IC₅₀═Kᵢ (1+S/Kₘ)+M [E] with ATP Kₘ for AKT1=78 μM. Compounds of Table 1 showed Kᵢ values for AKT1 ranging from about 80 nM to >10 μM, and the ratios of AKT1 Kᵢ to LATS2 Kᵢ of about 100 to over 100,000 folds for some of the compounds; and Kᵢ values for AKT1 ranging from about 26 nM to >10 μM, and the ratios of AKT1 Kᵢ to LATS2 Kᵢ of about 100 to over 500,000 folds for other compounds.

### Example B7: TAZ Nuclear Translocation Assay

The SW1990 cells (ATCC, CRL2172) were seeded at 5000 cells/well by adding 30 μL/well of cells in RPMI1640, 10% FBS, 2 mM glutamine culture media to a 384-well plate (Greiner 781091). The cells were incubated overnight at 37° C. Next day, test compounds were directly added to the cells using the Echo Liquid Handler (Labcyte), incubate at 37° C. for 4 hours. The cells were fixed with 4% paraformaldehyde and incubate for 20 minutes at room temperature. After washing three times with 100 μL PBS, 0.5% Triton X-100 was added to permeabilize the cells, incubate at room temperature for 5 minutes, following by washing the cells three times with PBS. 3% BSA was added to the cells, incubate for one hour at room temperature, following by washing three times with PBS. 50 μL of 1:750 diluted rabbit Anti-TAZ (Cell signaling D316D) in 3% BSA was added to the cells, incubate at 4° C. overnight. Next day, after washing the cells three times with PBS, 50 μL of 1:1250 diluted donkey anti-Rabbit, Alexa Fluor 488 (Invitrogen A21206) and 1:6250 diluted Hoechst 33342 (Molecular Probes H-21492) in 3% BSA was added to the cells, incubate at room temperature for one hour. The cells were washed three times with PBS, then image with the CellInsight CX7 High-Content Imager. The ratio of nuclear mean fluorescence intensity and cytoplasmic ring region mean fluorescence intensity was calculated. The % inhibition was normalized using DMSO as 0% inhibition and an inhibitor control as 100% inhibition. The EC₅₀ values were calculated by fitting the % inhibition with a nonlinear four-parameter logistical equation. The results are set forth in Table B1.

### Example B8: pYAP(Ser127) HTRF Cellular Assay

The SW1990 cells were seeded at 8000 cells/well by adding 30 μL/well of cells in RPMI1640, 10% FBS, 2 mM glutamine culture media to a 384-well plate (Corning 3570). The cells were incubated overnight at 37° C. Next day, compounds were directly added to the cells using the Echo Liquid Handler (Labcyte), incubate at 37° C. for 4 hours.

The HTRF assay was performed using the Cisbio phospho-YAP Ser127 HTRF kit (64YAPPEG). The media was aspirated from the cells. 20 μL of 1X lysis/blocking buffer was added to the cells, gently shake for 30 minutes at room temperature. 1 μL of the premixed antibody solution (1:40 dilution of each phospho-YAP Eu Cryptate antibody and phospho-YAP d2 antibody) was added to the cells. The plate was sealed and incubated overnight at room temperature. The HTRF (665 nm/620 nm) signal was read on the PHERAstar reader (BMG Labtech). The EC₅₀ values were calculated by fitting the % inhibition with a nonlinear four-parameter logistical equation. The results are set forth in Table B1.

TABLE B1

| | LATS1 | LATS2 | TAZ | pYAP |
|---|---|---|---|---|
| Compound | high ATP | HTRF | Translocation | EC₅₀ |
| No. | Kᵢ (nM) | Kᵢ (nM)¹ | EC₅₀ (μM) | (μM) |
| 101 | 0.051 | 0.38 | 0.99 | 1.1 |
| 102 | 0.011 | 0.98 | 0.35 | 0.43 |
| 103 | | 0.58 | 0.74 | |
| 104 | | 0.53 | 0.45 | |
| 105 | | 3.08 | 2.3 | 5.8 |
| 106 | | 0.053 | 0.3 | 0.23 |
| 107 | | 0.62 | 0.42 | 0.29 |
| 108 | | 0.31 | 0.38 | 0.58 |
| 109 | | 0.043 | 3.8 | |
| 110 | 0.0079 | 0.075 | 0.22 | 0.33 |
| 111 | | 0.14 | 0.63 | |
| 112 | | 1.07 | 0.7 | 0.94 |
| 113 | | 0.82 | 0.66 | 1.1 |
| 114 | | 0.31 | 0.61 | 0.55 |
| 115 | 0.0063 | 0.13 | 0.3 | 0.31 |
| 116 | 0.0079 | 0.13 | 0.31 | 0.32 |
| 117 | | 0.41 | 1.6 | |
| 118 | | 1.02* | >10 | |
| 119 | | 0.03 | 0.43 | |
| 120 | | 0.17 | 0.37 | 0.36 |
| 121 | | 0.8 | 0.3 | 0.63 |
| 122 | | 1.02* | 0.99 | |
| 123 | | 1.09 | 2.1 | |
| 124 | 0.0068 | 0.083 | 0.24 | 0.26 |
| 125 | | 16.66 | | |
| 126 | | 0.11 | 0.96 | |
| 127 | | 0.55 | 1.3 | 3.4 |
| 128 | 0.0058 | 0.073 | 0.3 | 0.36 |
| 129 | 0.0074 | 0.076 | 0.14 | 0.29 |
| 130 | | 0.45 | 1.2 | |
| 131 | | 0.29 | 0.28 | 0.49 |
| 132 | | 0.25 | 2.4 | |
| 133 | | 0.069 | 0.4 | 0.25 |
| 134 | | 0.72 | 1.1 | |
| 135 | | 0.063 | 0.42 | |
| 136 | | 0.45 | 3.2 | |
| 137 | | 2.17* | 1.7 | |
| 138 | | 0.61 | 0.59 | 0.89 |
| 139 | | 19.13 | | |
| 140 | | 88.44 | | |
| 141 | | 3.29 | >10 | |
| 142 | | 0.037 | >10 | |
| 143 | | 0.15 | 0.7 | 1 |
| 144 | | 0.086 | 0.69 | |
| 145 | | 0.039 | 0.94 | |
| 146 | | 0.088 | 2.7 | |
| 147 | 0.013 | 0.056 | 0.18 | 0.21 |
| 148 | | 0.12 | 0.56 | |
| 149 | | 0.045 | 2.5 | |
| 150 | | 0.059 | 0.28 | |
| 151 | | 0.09 | 0.47 | 0.39 |
| 152 | | 0.053 | 0.84 | |
| 153 | | 0.11 | >10 | |
| 154 | | 0.16* | 7.2 | |
| 155 | | 51.42 | | |
| 156 | | 0.7 | 4.6 | |
| 157 | 0.049 | 0.067 | 0.24 | 0.51 |
| 158 | | 1.29* | 3.1 | |
| 159 | | 6.58 | | |
| 160 | | 2.02 | >10 | |
| 161 | | 5.55 | 1.9 | |
| 162 | | 1.54 | 4.7 | |
| 163 | | 2.88 | >10 | |
| 164 | | 9.42*, 53.5 | >10 | |
| 165 | | 3.93*, 5.35 | 6.5 | |
| 166 | | 2.67 | 1.8 | |
| 167 | | 0.58 | 0.54 | 0.3 |
| 168 | | 1.62 | 0.83 | |
| 169 | | 0.45 | 0.57 | |
| 170 | | 0.29 | 0.38 | |
| 171 | | 1.95 | 8.4 | |
| 172 | | 0.64 | 6.5 | |
| 173 | | 7.4 | | |
| 174 | | 11.11 | | |
| 175 | | 22.62 | | |
| 176 | | 0.93 | 1.79 | |
| 177 | | 0.39 | 2.5 | |
| 178 | | 0.97 | | |
| 179 | | 0.10 | 0.74 | |
| 180 | | 1.38 | 1.3 | |
| 181 | | 1.87 | 1.9 | |
| 182 | | 2.26 | 2.7 | |
| 183 | | 17.5 | | |
| 184 | | 0.037 | 1.4 | |
| 185 | | 0.047 | 0.86 | |
| 186 | | 2.26 | >10 | |
| 187 | | 1.44 | >10 | |
| 188 | | 5.96 | | |
| 189 | | 0.76 | 0.54 | |
| 190 | | 0.026 | 0.11 | |
| 191 | | 0.028 | | |
| 192 | | 1.38 | | |
| 193 | | 0.041 | | |
| 194 | | 0.12 | | |
| 195 | | 0.039 | | |
| 196 | | 0.057 | 0.97 | |
| 197 | | 0.059 | 1.3 | 1.4 |
| 198 | | 0.041 | 1.4 | 2.4 |
| 199 | | 3.3 | 6.1 | |
| 200 | | 14.6 | | |
| 201 | | 11.3 | | |
| 203 | | 80.2 | | |
| 204 | | 1.1 | 1.0 | |
| 205 | | 0.23 | 0.17 | |
| 206 | | 0.10 | 0.17 | |
| 207 | | 23 | | |
| 208 | | 60 | | |
| 209 | | 45 | | |
| 210 | | 0.25 | | |
| 211 | | 0.11 | 0.42 | |
| 212 | | 0.41 | 2.5 | |
| 213 | | 0.60 | >10 | |
| 214 | | 10 | 0.85 | |
| 215 | | 1.2 | 1.5 | |
| 216 | | 0.13 | 0.49 | |
| 217 | | 0.35 | 0.78 | |
| 218 | | 4.3 | 6.8 | |
| 219 | | 0.067 | 0.46 | |
| 220 | | 0.074 | 0.66 | |
| 221 | | 0.32 | 0.23 | |
| 222 | | 0.1 | 0.34 | |
| 223 | | 2.7 | >10 | |
| 224 | | 0.16 | 8.9 | |
| 225 | | 2.1 | 1.8 | |
| 226 | | 0.016 | 0.13 | |
| 227 | | 0.086 | 2.3 | |
| 228 | | 0.067 | 2.7 | |
| 229 | | 0.063 | 2.1 | |
| 230 | | 0.043 | 1.0 | |
| 231 | | 0.11 | 0.9 | |
| 232 | | 0.061 | 4.3 | |
| 233 | | 0.028 | 0.26 | |
| 234 | | 4.5 | 4.2 | |
| 235 | | 1.3 | 1.8 | |
| 236 | | 0.49 | 1.2 | |
| 237 | | 19 | | |
| 238 | | 0.25 | 2.3 | |
| 239 | | 4.9 | >10 | |
| 240 | | 2.0 | 3.2 | |
| 241 | | 1.4 | >10 | |
| 242 | | 0.76 | 2.8 | |
| 243 | | 1.9 | 3.9 | |
| 244 | | 13 | | |
| 245 | | 0.039 | 4.8 | |
| 246 | | 3.5 | 3.1 | |
| 247 | | 0.039 | 0.31 | |
| 248 | | 0.02 | 0.11 | |
| 249 | | 0.27 | >10 | |
| 250 | | 0.033 | 0.10 | |
| 251 | | 3.5 | >10 | |
| 252 | | 0.022 | 0.13 | |
| 253 | | 0.28 | 0.12 | |
| 254 | | 0.018 | 0.10 | |
| 255 | | 0.034 | 0.14 | |
| 256 | | 0.19 | 0.47 | |
| 257 | | 8.0 | >10 | |
| 258 | | 1.8 | 1.9 | |
| 259 | | 1.6 | >10 | |
| 260 | | 0.055 | 4.0 | |
| 261 | | 0.10 | 0.58 | |
| 262 | | 0.028 | 0.25 | |
| 263 | | 0.11 | 1.2 | |
| 264 | | 0.026 | 0.17 | |
| 265 | | 0.047 | 0.24 | |
| 266 | | 0.37 | 1.1 | |
| 267 | | 0.028 | 0.36 | |
| 268 | | 0.034 | 0.13 | |
| 269 | | 0.034 | 0.21 | |
| 270 | | 0.15 | 0.86 | |
| 271 | | 0.041 | 0.33 | |
| 272 | | 0.31 | 1.1 | |
| 273 | | 0.043 | 0.26 | |
| 274 | | 0.02 | 0.18 | |
| 275 | | 0.02 | 0.31 | |
| 276 | | 0.19 | 0.41 | |
| 277 | | 0.11 | 0.29 | |
| 278 | | 11 | | |
| 279 | | 4.5 | 5.3 | |
| 280 | | 0.039 | 0.57 | |
| 281 | | 0.015 | | |
| 282 | | 0.039 | | |
| 283 | | 0.096 | | |
| 284 | | 4.1 | | |

¹Data with no asterisk was obtained using the LATS2 High ATP HTRF Assay. Data marked with an asterisk was obtained using the Standard LATS2 HTRF Assay.

It is to be noted that the term “a” or “an” entity refers to one or more of that entity; for example, “a polypeptide” is understood to represent one or more polypeptides. As such, the terms “a” (or “an”), “one or more,” and “at least one” can be used interchangeably herein.

All technical and scientific terms used herein have the same meaning. Efforts have been made to ensure accuracy with respect to numbers used (e.g. amounts, temperature, etc.) but some experimental errors and deviations should be accounted for.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

As used herein, the term “about,” when referring to a value is meant to encompass variations of, in some embodiments±50%, in some embodiments±20%, in some embodiments+10%, in some embodiments±5%, in some embodiments±1%, in some embodiments 0.5%, and in some embodiments±0.10% from the specified amount, as such variations are appropriate to perform the disclosed methods or employ the disclosed compositions.

Where a range of values is provided, it is understood that each intervening value, to the tenth of the unit of the lower limit, unless the context clearly dictates otherwise, between the upper and lower limit of the range and any other stated or intervening value in that stated range, is encompassed within the invention. The upper and lower limits of these small ranges which may independently be included in the smaller rangers is also encompassed within the invention, subject to any specifically excluded limit in the stated range. Where the stated range includes one or both of the limits, ranges excluding either or both of those included limits are also included in the invention.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A compound of Formula (I): 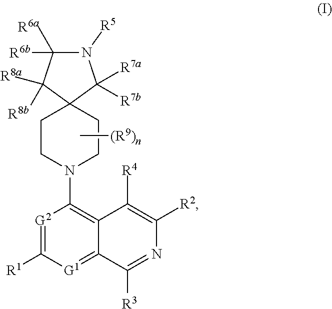
or an N-oxide thereof, or a pharmaceutically acceptable salt thereof, wherein:
R¹ is 5- to 14-membered heteroaryl optionally substituted with 1, 2, 3, 4 or 5 substituents independently selected from R¹⁰;
R² is hydrogen, halogen, C₁₋₆ alkyl, —O(C₁₋₆ alkyl), —NH(C₁₋₆ alkyl) or —N(C₁₋₆ alkyl)₂, wherein each C₁₋₆ alkyl is optionally substituted with 1, 2, 3, 4 or 5 substituents independently selected from R¹⁰;
R³ is hydrogen, C₁₋₆ alkyl, halogen, cyano, hydroxyl, —O(C₁₋₆ alkyl), C₂₋₆ alkenyl or C₂₋₆ alkynyl, wherein the C₁₋₆ alkyl, C₂₋₆ alkenyl and C₂₋₆ alkynyl are each optionally substituted with 1, 2, 3, 4 or 5 substituents independently selected from R¹⁰;
R⁴ is hydrogen, halogen, cyano, —NR^{43a}R^{43b}, —OR⁴⁴, C₁₋₆ alkyl or C₃₋₆ cycloalkyl, wherein the C₁₋₆ alkyl and C₃₋₆ cycloalkyl are each optionally substituted with 1, 2, 3, 4 or 5 substituents independently selected from R¹⁰;
G¹ is N or CR⁴¹, G² is N or CR⁴², provided that one or both of G¹ and G² are N;
R⁴¹ and R⁴² are independently hydrogen, halogen, cyano, —NR^{43a}R^{43b}, —OR⁴⁴, C₁₋₆ alkyl or C₃₋₆ cycloalkyl, wherein the C₁₋₆ alkyl and C₃₋₆ cycloalkyl are each optionally substituted with 1, 2, 3, 4 or 5 substituents independently selected from R¹⁰;
each R^{43a} and R^{43b} is independently hydrogen or C₁₋₆ alkyl;
R⁴⁴ is hydrogen, C₁₋₆ alkyl, C₂₋₆ alkenyl, C₂₋₆ alkynyl, C₃₋₈ cycloalkyl, 3- to 14-membered heterocyclyl, wherein the C₁₋₆ alkyl, C₂₋₆ alkenyl, C₂₋₆ alkynyl, C₃₋₈ cycloalkyl and 3- to 14-membered heterocyclyl of R⁴⁴ are each optionally substituted with 1, 2, 3 or 4 substituents independently selected from R¹⁰;
R⁵ is hydrogen, C₁₋₆ alkyl, C₂₋₆ alkenyl, C₂₋₆ alkynyl, C₃₋₈ cycloalkyl, C₆₋₁₄ aryl, 5- to 14-membered heteroaryl, 3- to 14-membered heterocyclyl, —C(O)R¹⁴, —C(O)OR¹⁵ or —C(O)NR^{16a}R^{16b}, wherein the C₁₋₆ alkyl, C₂₋₆ alkenyl, C₂₋₆ alkynyl, C₃₋₈ cycloalkyl, C₆₋₁₄ aryl, 5 to 14-membered heteroaryl and 3- to 14-membered heterocyclyl of R⁵ are each optionally substituted with 1, 2, 3, 4 or 5 substituents independently selected from R¹⁰; or is taken together with R^{6a} or R^{6b} and the atoms to which they are attached to form a 3- to 14-membered heterocyclyl optionally substituted with 1, 2, 3, 4 or 5 substituents independently selected from R¹⁰;
each R^{6a} and R^{6b} is independently hydrogen, C₁₋₆ alkyl, C₃₋₈ cycloalkyl, C₆₋₁₀ aryl, 5- to 14-membered heteroaryl, 3- to 12-membered heterocyclyl, —C(O)R¹⁴, —C(O)OR¹⁵ or —C(O)NR^{16a}R^{16b}, wherein the C₁₋₆ alkyl, C₃₋₈ cycloalkyl, C₆₋₁₀ aryl, 5- to 14-membered heteroaryl, and 3- to 12-membered heterocyclyl of R^{6a} and R^{6b} are each optionally substituted with 1, 2, 3, 4 or 5 substituents independently selected from R¹⁰; or taken together with R⁵ and the atoms to which they are attached to form a 3- to 14-membered heterocyclyl optionally substituted with 1, 2, 3, 4 or 5 substituents independently selected from R¹⁰; or R^{6a} and R^{6b} are taken together with the carbon to which they are attached to form a carbonyl;
each R^{7a} and R^{7b} is independently hydrogen or C₁₋₆ alkyl optionally substituted with 1, 2, 3, 4 or 5 substituents independently selected from R¹⁰; or R^{7a} and R^{7b} are taken together with the carbon to which they are attached to form a carbonyl;
each R^{8a} and R^{8b} is independently hydrogen, halogen, hydroxyl, —O(C₁₋₆ alkyl) or C₁₋₆ alkyl, wherein each C₁₋₆ alkyl is optionally substituted with 1, 2, 3, 4 or 5 substituents independently selected from R¹⁰;
n is 0 to 8;
each R⁹ is independently C₁₋₆ alkyl; or two geminal R^{g} groups, if present, are taken together with the carbon to which they are attached to form a carbonyl;
each R¹⁴ is independently hydrogen or C₁₋₆ alkyl;
each R¹⁵ is independently C₁₋₆ alkyl;
each R^{16a} and R^{16b} is independently hydrogen or C₁₋₆ alkyl; or R^{16a} and R^{16b} are taken together with the nitrogen atom to which they are attached to form a 4- to 12-membered heterocyclyl optionally substituted with 1, 2, 3, 4 or 5 substituents independently selected from R¹⁰;
each R¹⁰ is independently oxo, C₁₋₆ alkyl, C₂₋₆ alkenyl, C₂₋₆ alkynyl, C₃₋₈ cycloalkyl, C₆₋₁₄ aryl, 5- to 14-membered heteroaryl, 3- to 14-membered heterocyclyl, halogen, cyano, —C(O)R^{a}, —C(O)OR^{b}, —C(O)NR^{c}R^{d}, —OR^{b}, —OC(O)R^{a}, —OC(O)NR^{c}R^{d}, —SR^{b}, —S(O)R^{c}, —S(O)₂R^{c}, —S(O)(═NH)R^{c}, —S(O)₂NR^{c}R^{d}, —NR^{c}R^{d}, —N(R^{f})C(O)R^{a}, —N(R^{f})C(O)OR^{b}, —N(R^{f})C(O)NR^{c}R^{d}, —N(R^{f})S(O)₂R^{c}, —N(R^{f})S(O)₂NR^{c}R^{d} or —P(O)R^{g}R^{h}, wherein the C₁₋₆ alkyl, C₂₋₆ alkenyl, C₂₋₆ alkynyl, C₃₋₈ cycloalkyl, C₆₋₁₄ aryl, 5- to 14-membered heteroaryl and 3- to 14-membered heterocyclyl of R¹⁰ are each optionally substituted with 1, 2, 3 or 4 substituents independently selected from R¹¹;
each R^{a} is independently hydrogen, C₁₋₆ alkyl, C₂₋₆ alkenyl, C₂₋₆ alkynyl, C₃₋₈ cycloalkyl, C₆₋₁₀ aryl, 5- to 10-membered heteroaryl or 3- to 12-membered heterocyclyl, wherein the C₁₋₆ alkyl, C₂₋₆ alkenyl, C₂₋₆ alkynyl, C₃₋₈ cycloalkyl, C₆₋₁₀ aryl, 5- to 10-membered heteroaryl and 3- to 12-membered heterocyclyl of R^{a} are each optionally substituted with 1, 2, 3 or 4 substituents independently selected from R¹¹;
each R^{b} is independently hydrogen, C₁₋₆ alkyl, C₃₋₈ cycloalkyl, C₆₋₁₀ aryl, 5- to 10-membered heteroaryl or 3- to 12-membered heterocyclyl, wherein the C₁₋₆ alkyl, C₃₋₈ cycloalkyl, C₆₋₁₀ aryl, 5- to 10-membered heteroaryl and 3- to 12-membered heterocyclyl of R^{b} are each optionally substituted with 1, 2, 3 or 4 substituents independently selected from R¹¹;
each R^{c} and R^{d} is independently hydrogen, C₁₋₆ alkyl, C₃₋₈ cycloalkyl, C₆₋₁₀ aryl, 5- to 10-membered heteroaryl or 3- to 12-membered heterocyclyl; wherein the C₁₋₆ alkyl, C₃₋₈ cycloalkyl, C₆₋₁₀ aryl, 5- to 10-membered heteroaryl and 3- to 12-membered heterocyclyl of R^{c} and R^{d} are each optionally substituted with 1, 2, 3 or 4 substituents independently selected from R¹¹;
or R^{c} and R^{d} are taken together with the nitrogen atom to which they are attached to form a 4- to 12-membered heterocyclyl optionally substituted with 1, 2, 3 or 4 substituents independently selected from R¹¹;
each R^{c} is independently C₁₋₆ alkyl, C₃₋₈ cycloalkyl, C₆₋₁₀ aryl, 5- to 10-membered heteroaryl or 3- to 12-membered heterocyclyl, wherein the C₁₋₆ alkyl, C₃₋₈ cycloalkyl, C₆₋₁₀ aryl, 5- to 10-membered heteroaryl and 3- to 12-membered heterocyclyl of R^{c} are each optionally substituted with 1, 2, 3 or 4 substituents independently selected from R¹¹;
each R^{f2} is independently hydrogen or C₁₋₆ alkyl;
each R^{g} and R^{h} is independently C₁₋₆ alkyl, C₃₋₈ cycloalkyl, C₆₋₁₀ aryl, 5- to 10-membered heteroaryl, 3- to 12-membered heterocyclyl or —O—C₁₋₆ alkyl; wherein the C₁₋₆ alkyl, C₃₋₈ cycloalkyl, C₆₋₁₀ aryl, 5- to 10-membered heteroaryl and 3- to 12-membered heterocyclyl of R^{g} and R^{h} are each optionally substituted with 1, 2, 3 or 4 substituents independently selected from R¹¹;
or R^{g} and R^{h} are taken together with the phosphorus atom to which they are attached to form a 4- to 12-membered heterocyclyl optionally substituted with 1, 2, 3 or 4 substituents independently selected from R¹¹;
each R¹¹ is independently oxo, C₁₋₆ alkyl, C₂₋₆ alkenyl, C₂₋₆ alkynyl, C₃₋₆ cycloalkyl, C₆₋₁₀ aryl, 5- to 10-membered heteroaryl, 3- to 8-membered heterocyclyl, halogen, cyano, —C(O)R^{a1}, —C(O)OR^{b1}, —C(O)NR^{c1}R^{d1}, —OR¹¹, —OC(O)R^{a1}, —OC(O)NR^{c1}R^{d1}, —SR¹¹, —S(O)R^{c1}, —S(O)₂R^{c1}, —S(O)₂NR^{c1}R^{d1}, —NR^{c1}R^{d1}, —N(R^{f1})C(O)R^{a1}, —N(R¹¹)C(O)OR^{b1}, —N(R^{f1})C(O)NR^{c1}R^{d1}, —N(R^{f1})S(O)₂R^{c1}, —N(R^{f1})S(O)₂NR^{c1}R^{d1} or —P(O)R^{g1}R^{h1}; wherein the C₁₋₆ alkyl, C₂₋₆ alkenyl, C₂₋₆ alkynyl, C₃₋₆ cycloalkyl, C₆₋₁₀ aryl, 5- to 10-membered heteroaryl and 3- to 8-membered heterocyclyl of R¹¹ are each optionally substituted with 1, 2, 3 or 4 substituents independently selected from R¹²;
each R^{a1} is independently hydrogen, C₁₋₆ alkyl, C₂₋₆ alkenyl, C₂₋₆ alkynyl, C₃₋₆ cycloalkyl, C₆₋₁₀ aryl, 5- to 10-membered heteroaryl or 3- to 8-membered heterocyclyl; wherein the C₁₋₆ alkyl, C₂₋₆ alkenyl, C₂₋₆ alkynyl, C₃₋₆ cycloalkyl, C₆₋₁₀ aryl, 5- to 10-membered heteroaryl and 3- to 8-membered heterocyclyl of R^{a1} are each optionally substituted with 1, 2, 3 or 4 substituents independently selected from R¹²;
each R^{b1} is independently hydrogen, C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{b1} are each optionally substituted with 1, 2, 3 or 4 substituents independently selected from R¹²;
each R^{c1} and R^{d1} is independently hydrogen, C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{c1} and R^{d1} are each optionally substituted with 1, 2, 3 or 4 substituents independently selected from R¹²;
or R^{c1} and R^{d1} are taken together with the nitrogen atom to which they are attached to form a 4- to 8-membered heterocyclyl optionally substituted with 1, 2, 3 or 4 substituents independently selected from R¹²;
each R^{c1} is independently C₁₋₆ alkyl, C₃₋₆ cycloalkyl, C₆₋₁₀ aryl, 5- to 10-membered heteroaryl or 3- to 8-membered heterocyclyl; wherein the C₁₋₆ alkyl, C₃₋₆ cycloalkyl, C₆₋₁₀ aryl, 5- to 10-membered heteroaryl and 3- to 8-membered heterocyclyl of R^{c1} are each optionally substituted with 1, 2, 3 or 4 substituents independently selected from R¹²;
each R^{f1} is independently hydrogen or C₁₋₆ alkyl;
each R^{g1} and R^{h1} is independently C₁₋₆ alkyl, C₃₋₆ cycloalkyl, C₆₋₁₀ aryl, 5- to 10-membered heteroaryl, 3- to 8-membered heterocyclyl, or —O—C₁₋₆ alkyl; wherein the C₁₋₆ alkyl, C₃₋₆ cycloalkyl, C₆₋₁₀ aryl, 5- to 10-membered heteroaryl and 3- to 8-membered heterocyclyl of R^{g1} and R^{h1} are each optionally substituted with 1, 2, 3 or 4 substituents independently selected from R¹²;
or R^{g1} and R^{h1} are taken together with the phosphorus atom to which they are attached to form a 4- to 8-membered heterocyclyl optionally substituted with 1, 2, 3 or 4 substituents independently selected from R¹²;
each R¹² is independently oxo, C₁₋₆ alkyl, C₃₋₆ cycloalkyl, C₆ aryl, 5- to 6-membered heteroaryl, 3- to 6-membered heterocyclyl, halogen, cyano, —C(O)R^{a2}, —C(O)OR^{b2}, —C(O)NR^{c2}R^{d2}, —OR^{b2}, —OC(O)R^{a2}, —OC(O)NR^{c2}R^{d2}, —S(O)₂R^{c2}, —S(O)₂NR^{c2}R^{d2}, —NR^{c2}R^{d2}, —N(R^{f})C(O)R^{a2}, —N(R^{f})C(O)OR^{b2}, —N(R^{f})C(O)NR^{c2}R^{d2}, —N(R^{f2})S(O)₂R^{c2}, —N(R^{f})S(O)₂NR^{c2}R^{d2} or —P(O)R^{g2}R^{h2}; wherein the C₁₋₆ alkyl, C₃₋₆ cycloalkyl, C₆ aryl, 5- to 6-membered heteroaryl and 3- to 6-membered heterocyclyl of R¹² are each optionally substituted with 1, 2, 3 or 4 substituents independently selected from R¹³;
each R^{a2} is independently hydrogen, C₁₋₆ alkyl, C₃₋₆ cycloalkyl, C₆ aryl, 5- to 6-membered heteroaryl or 3- to 6-membered heterocyclyl; wherein the C₁₋₆ alkyl, C₃₋₆ cycloalkyl, C₆ aryl, 5- to 6-membered heteroaryl and 3- to 6-membered heterocyclyl of R^{a2} are each optionally substituted with 1, 2, 3 or 4 substituents independently selected from R¹³;
each R^{b2} is independently hydrogen, C₁₋₆ alkyl, C₃₋₆ cycloalkyl or 3- to 6-membered heterocyclyl; wherein the C₁₋₆ alkyl, C₃₋₆ cycloalkyl and 3- to 6-membered heterocyclyl of R^{b2} are each optionally substituted with 1, 2, 3 or 4 substituents independently selected from R¹³;
each R^{c2} and R^{d2} is independently hydrogen, C₁₋₆ alkyl, C₃₋₆ cycloalkyl or 3- to 8-membered heterocyclyl; wherein the C₁₋₆ alkyl, C₃₋₆ cycloalkyl and 3- to 8-membered heterocyclyl of R^{c2} and R^{d2} are each optionally substituted with 1, 2, 3 or 4 substituents independently selected from R¹³;
or R^{c2} and R^{d2} are taken together with the nitrogen atom to which they are attached to form a 4- to 6-membered heterocyclyl optionally substituted with 1, 2, 3 or 4 substituents independently selected from R¹³;
each R^{c2} is independently C₁₋₆ alkyl, C₃₋₆ cycloalkyl, C₆ aryl, 5- to 6-membered heteroaryl or 3- to 6-membered heterocyclyl; wherein the C₁₋₆ alkyl, C₃₋₆ cycloalkyl, C₆ aryl, 5 to 6-membered heteroaryl and 3- to 6-membered heterocyclyl of R^{c2} are each optionally substituted with 1, 2, 3 or 4 substituents independently selected from R¹³;
each R^{f2} is independently hydrogen or C₁₋₆ alkyl;
each R^{g2} and R^{h2} is independently C₁₋₆ alkyl, C₃₋₆ cycloalkyl, 3- to 8-membered heterocyclyl, or —O—C₁₋₆ alkyl; wherein the C₁₋₆ alkyl, C₃₋₆ cycloalkyl, and 3- to 8-membered heterocyclyl of R^{g2} and R^{h2} are each optionally substituted with 1, 2, 3 or 4 substituents independently selected from R¹³;
or R^{g2} and R^{h2} are taken together with the phosphorus atom to which they are attached to form a 4- to 6-membered heterocyclyl optionally substituted with 1, 2, 3 or 4 substituents independently selected from R¹³; and
each R¹³ is independently oxo, halogen, hydroxyl, —O(C₁₋₆ alkyl), cyano, C₁₋₆ alkyl or C₁₋₆ haloalkyl;
provided that the compound is other than a compound in Table 1X and salts thereof.

2. The compound of claim 1, wherein both G¹ and G² are N, and the compound is of the Formula (IA): 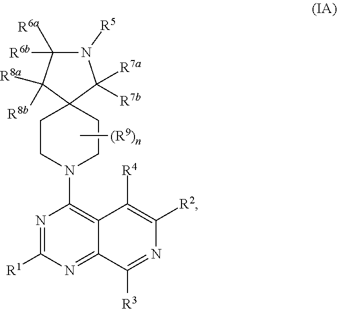
or a pharmaceutically acceptable salt thereof, wherein R¹, R², R³, R⁴, R⁵, R^{6a}, R^{6b}, R^{7a}, R^{7b}, R^{8a}, R^{8b}, R⁹ and n are as defined in claim 1.

3. The compound of claim 1, wherein G¹ is N and G² is CR⁴², and the compound is of the Formula (IB): 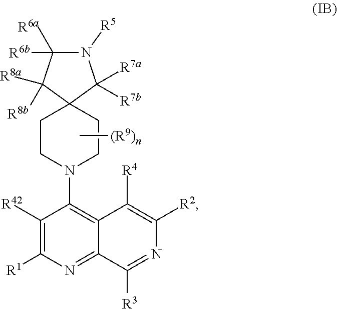
or a pharmaceutically acceptable salt thereof, wherein R¹, R², R³, R⁴, R⁴², R⁵, R^{6a}, R^{6b}, R^{7a}, R^{7b}, R^{8a}, R^{8b}, R⁹ and n are as defined in claim 1.

4. The compound of claim 1, wherein G¹ is CR⁴¹ and G² is N, and the compound is of the Formula (IC): 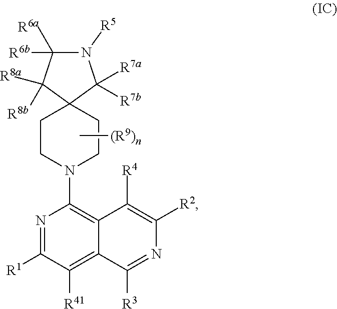
or a pharmaceutically acceptable salt thereof, wherein R¹, R², R³, R⁴, R⁴¹, R⁵, R^{6a}, R^{6b}, R^{7a}, R^{7b}, R^{8a}, R^{8b}, R⁹ and n are as defined in claim 1.

5. The compound of any one of claims 1 to 4, wherein R¹ is 6-membered heteroaryl having 1 or 2 ring nitrogen atoms, optionally substituted with 1, 2, 3 or 4 substituents independently selected from R¹⁰.

6. The compound of claim 5, wherein R¹ is 4-pyridyl optionally substituted with 1 to 5 substituents independently selected from R¹⁰.

7. The compound of any one of claims 1 to 4, wherein R¹ is 5-membered heteroaryl having 1 or 2 ring nitrogen atoms, optionally substituted with 1, 2, 3 or 4 substituents independently selected from R¹⁰.

8. The compound of claim 7, wherein R¹ is isothiazol-5-yl or pyrazol-4-yl optionally substituted with 1 to 3 substituents independently selected from R¹⁰.

9. The compound of any one of claims 1 to 4, wherein R¹ is 5,6-fused heteroaryl having 1 or 2 ring nitrogen atoms, optionally substituted with 1, 2, 3, 4 or 5 substituents independently selected from R¹⁰.

10. The compound of any one of claims 1 to 4, wherein R¹ is selected from the group consisting of: 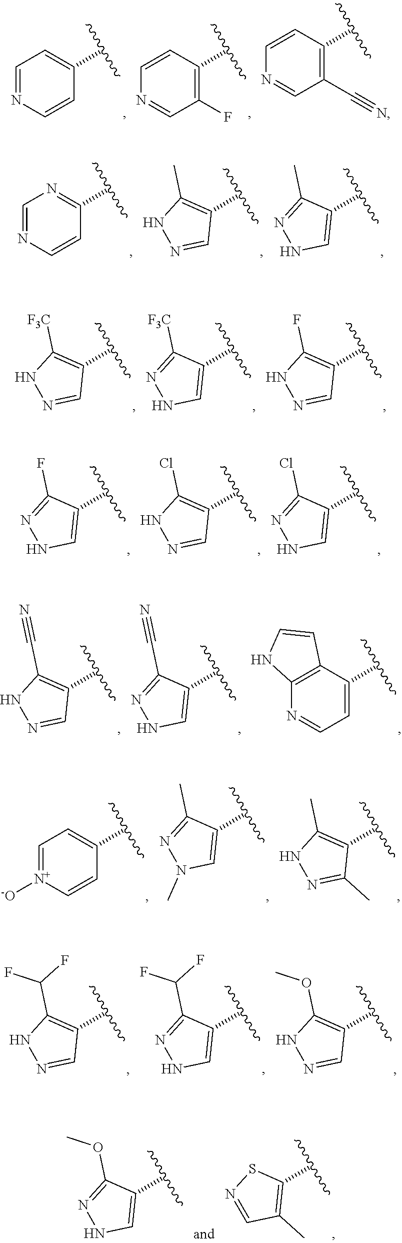
wherein the wavy line in each group indicates the point of attachment to the parent structure.

11. The compound of any one of claims 1 to 10, wherein R² is hydrogen, —NH(C₁₋₆ alkyl), or C₁₋₆ alkyl optionally substituted with 1, 2, 3, 4 or 5 substituents independently selected from R¹⁰.

12. The compound of claim 11, wherein R² is selected from the group consisting of hydrogen, NHMe, methyl, CF₃, 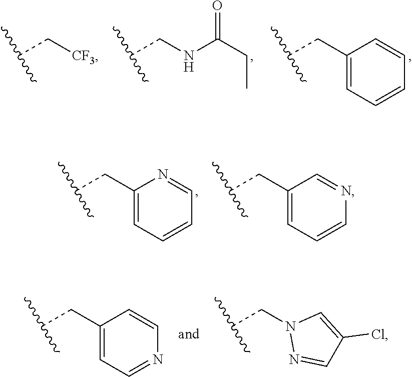
wherein the wavy line in each group indicates the point of attachment to the parent structure.

13. The compound of any one of claims 1 to 12, wherein R³ is hydrogen, halogen, cyano, hydroxyl, —O(C₁₋₆ alkyl), C₁₋₆ alkyl or C₂₋₆ alkynyl, wherein the C₁₋₆ alkyl and C₂₋₆ alkynyl are each optionally substituted with 1, 2, 3, 4 or 5 substituents independently selected from R¹⁰.

14. The compound of claim 13, wherein R³ is selected from the group consisting of hydrogen, chloro, cyano, hydroxyl, methoxy, 3-hydroxyprop-1-yn-1-yl, 3-hydroxy-3-methylbut-1-yn-1-yl, methyl, methoxymethyl and 2,2,2-trifluoroethyl.

15. The compound of any one of claims 1 to 14, wherein R 4 is hydrogen, halogen, —NR^{43a}R^{43b}), —OR⁴⁴, C₁₋₆ alkyl or C₃₋₆ cycloalkyl, wherein the C₁₋₆ alkyl and C₃₋₆ cycloalkyl are each optionally substituted with 1, 2, 3, 4 or 5 substituents independently selected from Rm.

16. The compound of claim 15, wherein R 4 is hydrogen, halogen, C₁₋₆ alkyl or —O(C₁₋₆ alkyl), wherein each C₁₋₆ alkyl is optionally substituted with 1, 2, 3, 4 or 5 substituents independently selected from R¹⁰.

17. The compound of claim 15, wherein R⁴ is selected from the group consisting of hydrogen, fluoro, chloro, bromo, methyl,

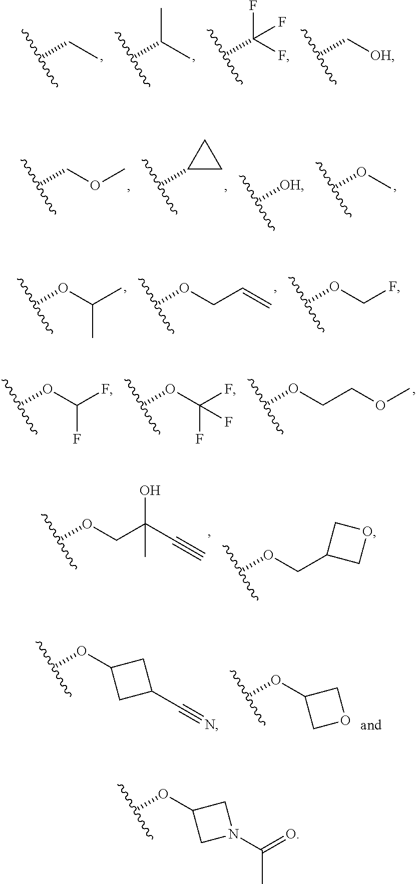

18. The compound of any one of claims 1 to 17, wherein R⁵ is hydrogen, C₁₋₆ alkyl, C₃₋₈ cycloalkyl, C₆₋₁₄ aryl, 5- to 14-membered heteroaryl, 3- to 14-membered heterocyclyl or —C(O)R¹⁴, wherein the C₁₋₆ alkyl, C₃₋₈ cycloalkyl, C₆₋₁₄ aryl, 5- to 14-membered heteroaryl and 3 to 14-membered heterocyclyl of R⁵ are each optionally substituted with 1, 2, 3, 4 or 5 substituents independently selected from R¹⁰.

19. The compound of claim 18, wherein R⁵ is hydrogen or —C(O)R¹⁴.

20. The compound of claim 18, wherein R⁵ is hydrogen or C₁₋₆ alkyl optionally substituted with 1, 2, 3, 4 or 5 substituents independently selected from R¹⁰.

21. The compound of claim 20, wherein R⁵ is selected from the group consisting of methyl, ethyl, 1-propyl, 2-propyl, 2-methyl-1-propyl and 2-methyl-2-propyl, 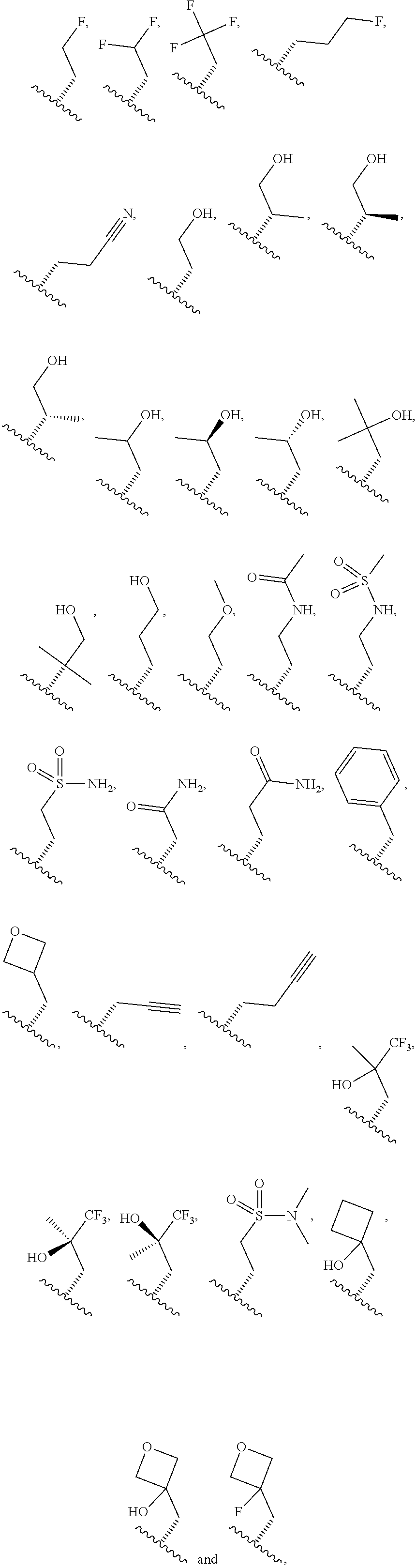
wherein the wavy line in each group indicates the point of attachment to the parent structure.

22. The compound of claim 18, wherein R⁵ is C₄₋₈ cycloalkyl optionally substituted with 1, 2, 3, 4 or 5 substituents independently selected from R¹⁰.

23. The compound of claim 22, wherein R⁵ is selected from the group consisting of 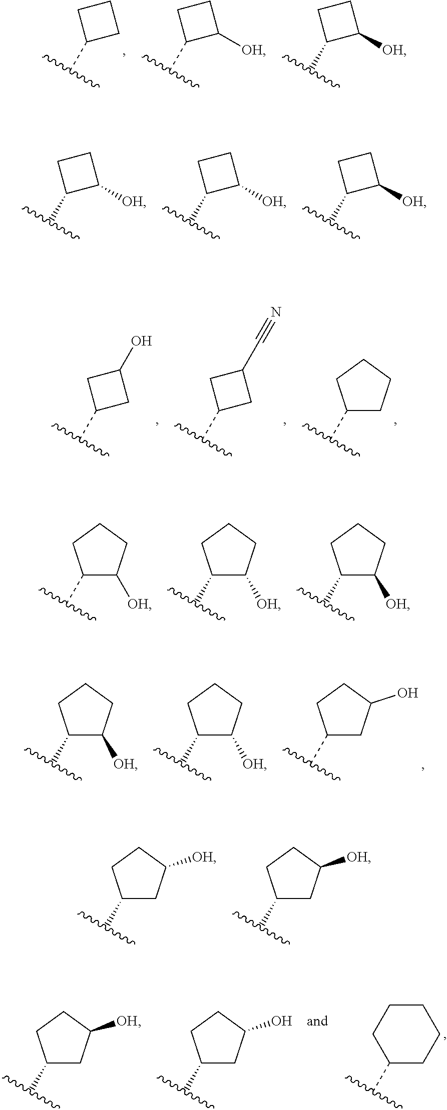
wherein the wavy line in each group indicates the point of attachment to the parent structure.

24. The compound of claim 17, wherein R⁵ is 3- to 14-membered heterocyclyl optionally substituted with 1, 2, 3, 4 or 5 substituents independently selected from R¹⁰, C₆₋₁₄ aryl optionally substituted with 1, 2, 3, 4 or 5 substituents independently selected from R¹⁰, or 5- to 14-membered heteroaryl optionally substituted with 1, 2, 3, 4 or 5 substituents independently selected from R¹⁰.

25. The compound of claim 24, wherein R⁵ is selected from the group consisting of 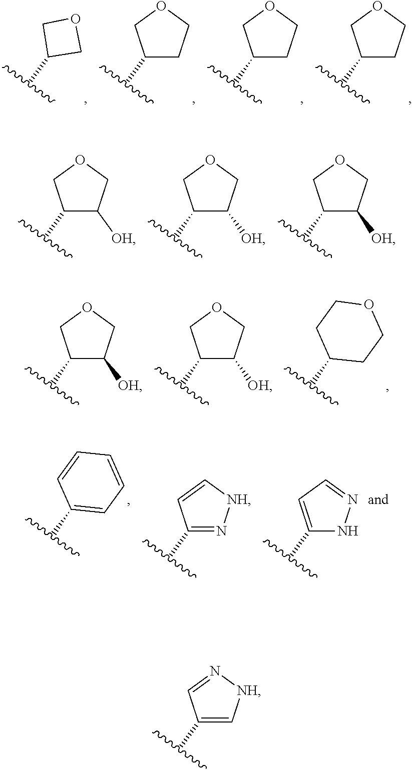
each of which is optionally substituted with 1, 2, 3, 4 or 5 substituents independently selected from R¹⁰, wherein the wavy line in each group indicates the point of attachment to the parent structure.

26. The compound of any one of claims 1 to 25, wherein each R^{6a} and R^{6b} is independently hydrogen or C₁₋₆ alkyl; or R^{6a} and R^{6b} are taken together with the carbon to which they are attached to form a carbonyl.

27. The compound of any one of claims 1 to 25, wherein one of R^{6a} and R^{6b} is hydrogen and the other one of R^{6a} and R^{6b} is hydrogen, —C(O)OR¹⁵, —C(O)NR^{16a}R^{16b} or C₁₋₆ alkyl optionally substituted with 1, 2, 3, 4 or 5 substituents independently selected from R¹⁰.

28. The compound of claim 27, wherein one of R^{6a} and R^{6b} is hydrogen and the other one of R^{6a} and R^{6b} is —C(O)OR¹⁵ or —C(O)NR^{16a}R^{16b}; wherein each R^{16a} and R^{16b} is independently hydrogen or C₁₋₆ alkyl.

29. The compound of claim 27, wherein one of R^{6a} and R^{6b} is hydrogen and the other one of R^{6a} and R^{6b} is —C(O)NR^{16a}R^{16b}; wherein R^{16a} and R^{16b} are taken together with the nitrogen atom to which they are attached to form a 4- to 12-membered heterocyclyl optionally substituted with 1, 2, 3, 4 or 5 substituents independently selected from R¹⁰.

30. The compound of claim 27, wherein one of R^{6a} and R^{6b} is hydrogen and the other one of R^{6a} and R^{6b} is selected from the group consisting of hydrogen, methyl, 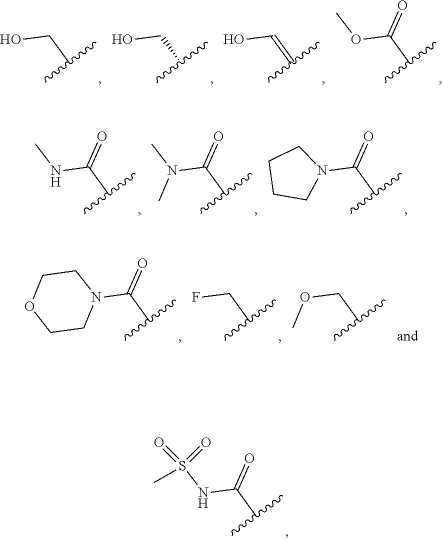
wherein the wavy line in each group indicates the point of attachment to the parent structure.

31. The compound of any one of claims 1 to 17, wherein R⁵ is taken together with one of R^{6a} and R^{6b} and the atoms to which they are attached to form a 3- to 14-membered heterocyclyl optionally substituted with 1, 2, 3, 4 or 5 substituents independently selected from R¹⁰, and the other one of R^{6a} and R^{6b} is hydrogen, C₁₋₆ alkyl, C₃₋₈ cycloalkyl, C₆₋₁₀ aryl, 5- to 14-membered heteroaryl, 3- to 12-membered heterocyclyl, —C(O)R¹⁴, —C(O)OR¹⁵ or —C(O)NR^{16a}R^{16b}, wherein the C₁₋₆ alkyl, C₃₋₈ cycloalkyl, C₆₋₁₀ aryl, 5- to 14-membered heteroaryl, and 3- to 12-membered heterocyclyl are each optionally substituted with 1, 2, 3, 4 or 5 substituents independently selected from R¹⁰.

32. The compound of claim 31, wherein R⁵ is taken together with one of R^{6a} and R^{6b} and the atoms to which they are attached to form a 4- to 8-membered heterocyclyl optionally substituted with 1, 2, 3, 4 or 5 substituents independently selected from R¹⁰, and the other one of R^{6a} and R^{6b} is hydrogen.

33. The compound of any one of claims 1 to 32, wherein each R^{7a} and R^{7b} is independently hydrogen or C₁₋₆ alkyl; or R^{7a} and R^{7b} are taken together with the carbon to which they are attached to form a carbonyl.

34. The compound of any one of claims 1 to 33, wherein one of R^{8a} and R^{8b} is hydrogen, and the other one of R^{8a} and R^{8b} is hydrogen, halogen, hydoxyl, C₁₋₆ alkyl, or —O(C₁₋₆ alkyl).

35. The compound of claim 32, wherein each R^{8a} and R^{8b} is hydrogen; one of R^{8a} and R^{8b} is hydrogen and the other one of R^{8a} and R^{8b} is hydrogen, fluoro or hydroxyl; or each R^{8a} and R^{8b} is fluoro.

36. The compound of any one of claims 1 to 35, wherein n is 0.

37. The compound of any one of claims 1 to 36, wherein the compound is of the Formula (II): 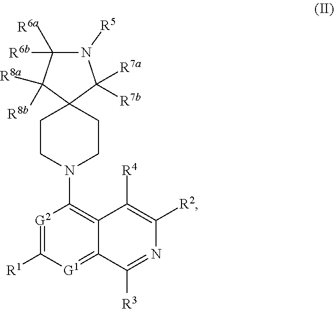
or a pharmaceutically acceptable salt thereof, wherein R¹, R², R³, R⁴, G², R⁵, R^{6a}, R^{6b}, R^{7a}, R^{7b}, R^{8a} and R^{8b} are as defined in any one of claims 1 to 32.

38. The compound of claim 37, wherein both G¹ and G² are N, and the compound is of the Formula (II-A): 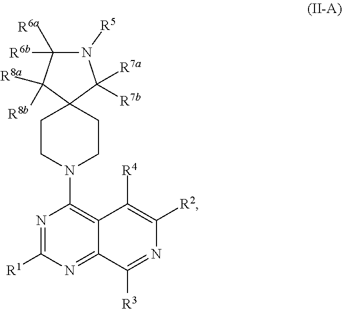
or a pharmaceutically acceptable salt thereof, wherein R¹, R², R³, R⁴, R⁵, R^{6a}, R^{6b}, R^{7a}, R^{7b}, R^{8a} and R^{8b} are as defined in claim 37.

39. The compound of claim 37, wherein G¹ is N and G² is CR⁴², and the compound is of the Formula (II-B): 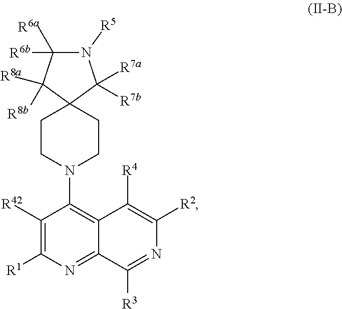
or a pharmaceutically acceptable salt thereof, wherein R¹, R², R³, R⁴, R⁴², R⁵, R^{6a}, R^{6b}, R^{7a}, R^{7b}, R^{8a} and R^{8b} are as defined in claim 37.

40. The compound of claim 37, wherein G¹ is CR⁴¹ and G² is N, and the compound is of the Formula (II-C): 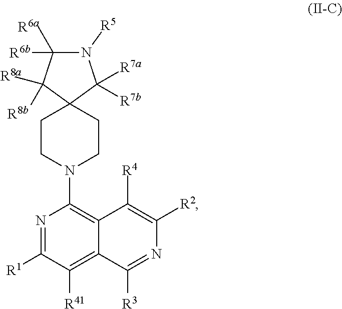
or a pharmaceutically acceptable salt thereof, wherein R¹, R², R³, R⁴, R⁴¹, R⁵, R^{6a}, R^{6b}, R^{7a}, R^{7b}, R^{8a} and R^{8b} are as defined in claim 37.

41. The compound of any one of claims 37 to 40, wherein:
R¹ is pyrazolyl, pyridinyl, isothiazolyl or pyrrolo-pyridinyl, each of which is optionally substituted with 1 to 3 substituents independently selected from R¹⁰;
R² is hydrogen or C₁₋₆ alkyl optionally substituted with 1 to 5 substituents independently selected from R¹⁰;
R³ is hydrogen, halogen or C₁₋₆ alkyl;
R⁴ is hydrogen, halogen, —O(C₁₋₆ alkyl) or C₁₋₆ alkyl;
R⁵ is hydrogen, C₁₋₆ alkyl, C₃₋₈ cycloalkyl, C₆₋₁₄ aryl, 5- to 14-membered heteroaryl, 3- to 14-membered heterocyclyl or —C(O)R¹⁴, wherein the C₁₋₆ alkyl, C₃₋₈ cycloalkyl, C₆₋₁₄ aryl, 5- to 14-membered heteroaryl and 3- to 14-membered heterocyclyl of R⁵ are each optionally substituted with 1, 2, 3, 4 or 5 substituents independently selected from R¹⁰;
each R^{6a} and R^{6b} is independently hydrogen, —C(O)OR¹⁵, —C(O)NR^{16a}R^{16b} or C₁₋₆ alkyl optionally substituted with 1, 2, 3, 4 or 5 substituents independently selected from R¹⁰, or R^{6a} and R^{6b} are taken together with the carbon to which they are attached to form a carbonyl;
each R^{7a} and R^{7b} is independently hydrogen or C₁₋₆ alkyl, or R^{7a} and R^{7b} are taken together with the carbon to which they are attached to form a carbonyl;
one of R^{8a} and R^{8b} is hydrogen, and the other one of R^{8a} and R^{8b} is hydrogen, halogen, hydoxyl, C₁₋₆ alkyl, or —O(C₁₋₆ alkyl);
R¹⁴ is C₁₋₆ alkyl;
R¹⁵ is C₁₋₆ alkyl; and
each R^{16a} and R^{16b} is independently hydrogen or C₁₋₆ alkyl, or R^{16a} and R^{16b} are taken together with the nitrogen atom to which they are attached to form a 5- or 6-membered heterocyclyl having 1 to 2 annular heteroatoms selected from nitrogen, oxygen and sulfur, optionally substituted with 1, 2, 3, 4 or 5 substituents independently selected from R¹⁰.

42. The compound of any one of claims 37 to 40, wherein:
R¹ is pyrazol-4-yl, 4-pyridyl, isothiazol-5-yl or pyrrolo[2,3-b]pyridin-4-yl, each of which is optionally substituted with 1 to 3 substituents independently selected from the group consisting of halogen, cyano, unsubstituted C₁₋₆ alkyl and C₁₋₆ haloalkyl;
each R² and R³ is independently hydrogen or C₁₋₆ alkyl;
R⁴ is hydrogen, halogen, —O(C₁₋₆ alkyl) or C₁₋₆ alkyl;
R⁵ is (i) C₁₋₆ alkyl optionally substituted with 1, 2, 3, 4 or 5 substituents independently selected from the group consisting of halogen, cyano, hydroxyl, —O(C₁₋₆ alkyl), —NHC(O)(C₁₋₆ alkyl), —NHS(O)₂(C₁₋₆ alkyl), —S(O)₂NH₂, —C(O)NH₂, phenyl and 3- to 12-membered heterocyclyl,
(ii) C₃₋₆ cycloalkyl substituted with 1, 2, 3, 4 or 5 substituents independently selected from the group consisting of halogen, cyano and hydroxyl,
(iii) monocyclic 3- to 6-membered heterocyclyl having 1 annular heteroatom which is oxygen,
(iv) phenyl, or
(v) pyrazolyl;
one of R^{6a} and R^{6b} is hydrogen, and the other one of R^{6a} and R^{6b} is hydrogen, C₁₋₆ alkyl, —C(O)O(C₁₋₆ alkyl) or —C(O)NR^{16a}R^{16b}, or R^{6a} and R^{6b} are taken together with the carbon to which they are attached to form a carbonyl;
one of R^{7a} and R^{7b} is hydrogen, and the other one of R^{7a} and R^{7b} is hydrogen or C₁₋₆ alkyl, or R^{7a} and R^{7b} are taken together with the carbon to which they are attached to form a carbonyl;
R^{8a} and R^{8b} are hydrogen; and
each R^{16a} and R^{16b} is independently hydrogen or C₁₋₆ alkyl, or R^{16a} and R^{16b} are taken together with the nitrogen atom to which they are attached to form pyrrolidin-1-yl or morpholin-4-yl.

43. The compound of any one of claims 1 to 36, wherein the compound is of the Formula (III): 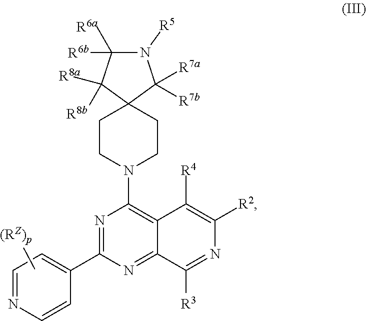
or a pharmaceutically acceptable salt thereof, wherein R², R³, R⁴, R⁵, R^{6a}, R^{6b}, R^{7a}, R^{7b}, R^{8a} and R^{8b} are as defined, where applicable, in any one of claims 1 to 36;
p is 0, 1, 2, 3 or 4; and
each R^{Z} is independently hydrogen, halogen, cyano or C₁₋₆ alkyl optionally substituted with 1, 2, 3, 4 or 5 substituents independently selected from R¹⁰.

44. The compound of claim 43, wherein p is 0, and the compound is of the Formula (V): 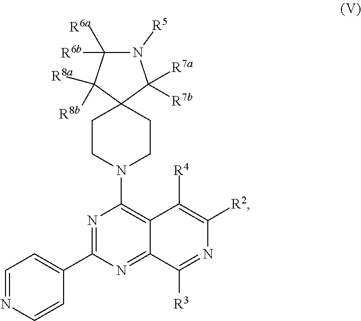
or a pharmaceutically acceptable salt thereof.

45. The compound of any one of claims 1 to 36, wherein the compound is of the Formula (IV): 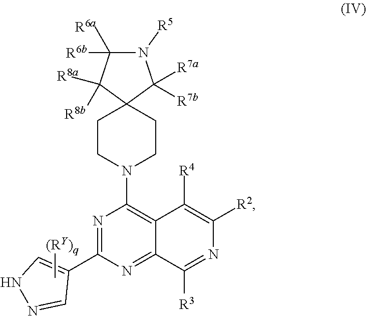
or a pharmaceutically acceptable salt thereof, wherein R², R³, R⁴, R⁵, R^{6a}, R^{6b}, R^{7a}, R^{7b}, R^{8a} and R^{8b} are as defined, where applicable, in any one of claims 1 to 36;
q is 0, 1, 2 or 3; and
each R^{Y} is independently hydrogen, halogen, cyano, —O(C₁₋₆ alkyl) or C₁₋₆ alkyl, wherein the C₁₋₆ alkyl of R is optionally substituted with 1, 2, 3, 4 or 5 substituents independently selected from R¹⁰.

46. The compound of claim 45, wherein q is 1, and R^{Y} is methyl, fluoro, chloro, cyano or trifluoromethyl.

47. The compound of any one of claims 1 to 36, wherein the compound is of the Formula (VII) or (VIII): 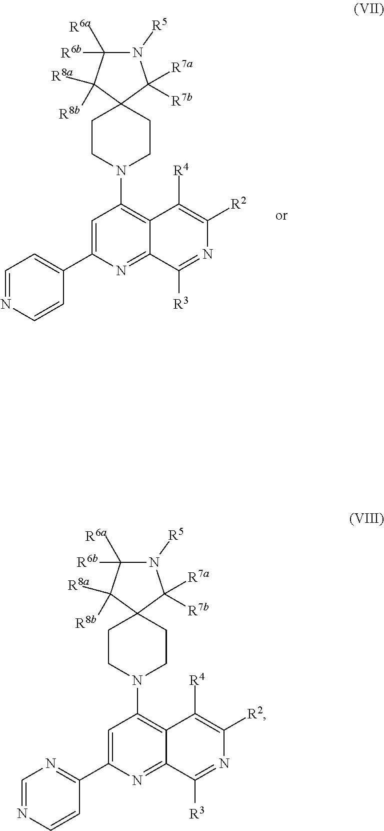
or a pharmaceutically acceptable salt thereof, wherein R², R³, R⁴, R⁵, R^{6a}, R^{6b}, R^{7a}, R^{7b}, R^{8a} and R^{8b} are as defined, where applicable, in any one of claims 1 to 36.

48. The compound of any one of claims 1 to 36, wherein the compound is of the Formula (IX): 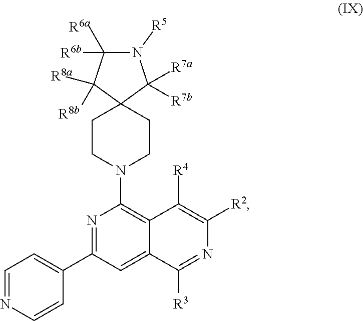
or a pharmaceutically acceptable salt thereof, wherein R², R³, R⁴, R⁵, R^{6a}, R^{6b}, R^{7a}, R^{7b}, R^{8a} and R^{8b} are as defined, where applicable, in any one of claims 1 to 36.

49. The compound of any one of claims 1 to 48, wherein each R², R³ and R⁴ is hydrogen.

50. The compound of claim 1, wherein the compound is selected from the group consisting of Compound Nos. 101 to 292 in Table 1, or a pharmaceutically acceptable salt thereof.

51. A pharmaceutical composition comprising the compound of any one of claims 1 to 50, or a pharmaceutically acceptable salt thereof, and a pharmaceutically acceptable excipient.

52. A method for making a compound of Formula (I): 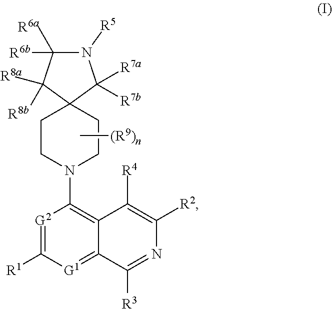
or a pharmaceutically acceptable salt thereof, wherein R¹, R², R³, R⁴, G¹, G², R⁵, R^{6a}, R^{6b}, R^{7a}, R^{7b}, R^{8a}, R^{8b}, R⁹, and n are as defined in claim 1, the method comprising
a) reacting a compound of Formula (I-4): 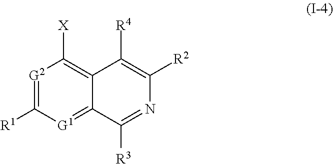
wherein X is selected from the group consisting of Cl, Br, I, F, OMs, and OTs, with a compound of Formula (I-3): 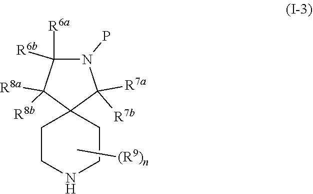
wherein P is a protecting group, in the presence of a base to produce a compound of Formula (I-2): 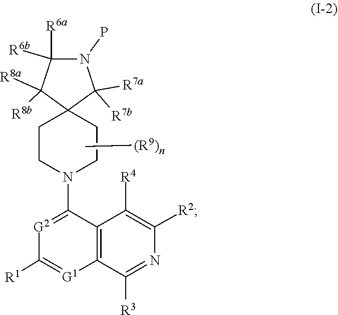
b) removing protecting group P from the compound of Formula (I-2) to produce a compound of Formula (I-1): 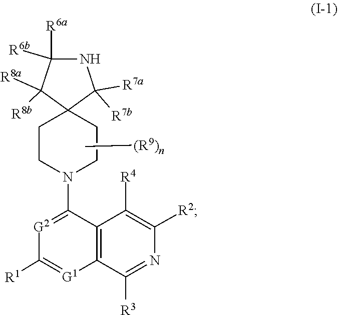
and
c) converting the compound of Formula (I-1) to the compound of Formula (I).

53. The method of claim 52, wherein the compound of Formula (I-1) is converted to the compound of Formula (I) (i) by contacting the compound of Formula (I-1) with a compound of the formula R⁵—X′, wherein X′ is a leaving group, in the presence of a base; or (ii) by contacting the compound of Formula (I-1) with an aldehyde of the formula R⁵—CHO in the presence of a reducing agent.

54. The method of claim 52, wherein both G¹ and G² are N, and the method further comprising:
d) mixing a compound of Formula 1 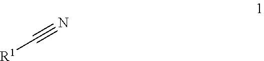
with a compound of Formula 2: 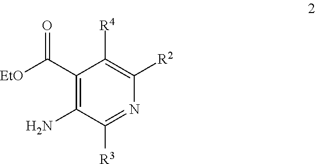
in the presence of a base to form a compound of Formula 3: 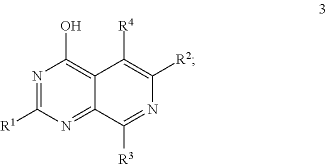
and
e) contacting the compound of Formula 3 with an activating agent to form the compound of Formula (I-4) wherein both G¹ and G² are N.

55. A method for making a compound of formula (IA): 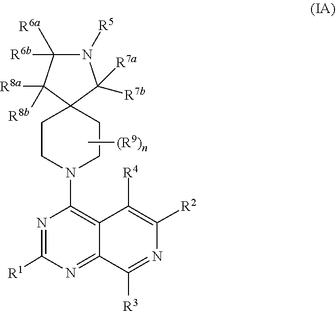
or a pharmaceutically acceptable salt thereof, wherein R¹, R², R³, R⁴, R⁵, R^{6a}, R^{6b}, R^{7a}, R^{7b}, R^{8a}, R^{8b}, R⁹, and n are as defined in claim 1, the method comprising:
a) contacting a compound of Formula 12: 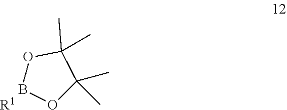
with a compound of Formula 11: 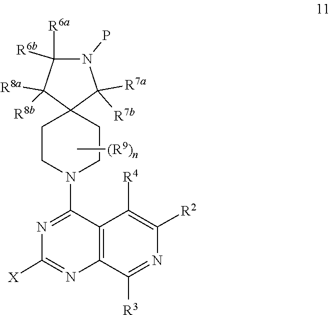
in the presence of a palladium catalyst and a base to form a compound of Formula 6: 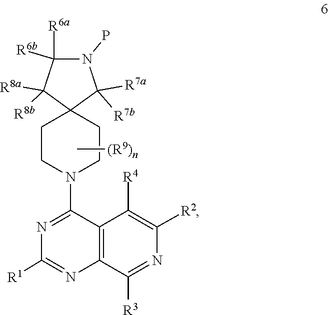
wherein X is selected from the group consisting of Cl, Br, I, F, OMs, and OTs, and P is a protecting group;
b) removing protecting group P from the compound of Formula 6 to produce a compound of Formula 7: 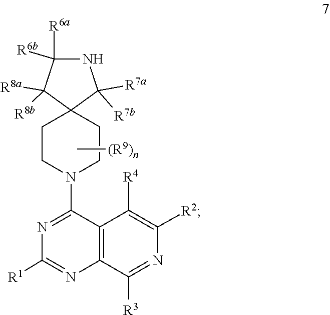
and
c) converting the compound of Formula 7 to the compound of Formula (IA).

56. The method of claim 55 further comprising:
d) coupling a compound of Formula 5: 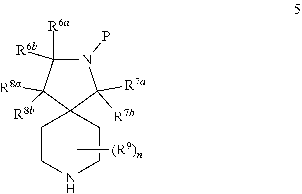
to a compound of Formula 10: 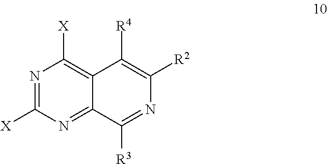
wherein X is selected from the group consisting of Cl, Br, I, F, OMs, and OTs, in the presence of a base to form the compound of Formula 11.

57. The method of claim 55 or 56, wherein the compound of Formula 7 is converted to the compound of Formula (IA) (i) by contacting the compound of Formula 7 with a compound of Formula 8: 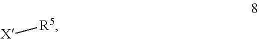
wherein X′ is a leaving group, in the presence of a base; or
(ii) by contacting the compound of Formula 7 with a compound of Formula 9 in the presence of a reducing agent:

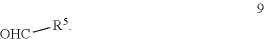

58. A method of inhibiting LATS1/2 in a cell, comprising contacting the cell with the compound of any one of claims 1 to 50, or a pharmaceutically acceptable salt thereof; or the pharmaceutical composition of claim 51.

59. A method for treating a disease or condition, said method comprising administering to a subject in need thereof an effective amount of the compound of any one of claims 1 to 50, or a pharmaceutically acceptable salt thereof; or the pharmaceutical composition of claim 51.

60. The method of claim 54, wherein the disease or condition is acute respiratory distress syndrome (ARDS).
